(19) United States
(12) Patent Application Publication
(10) Pub. No.: US 2024/0109955 A1
(43) Pub. Date: Apr. 4, 2024

(54) ANTI-SARS-COV-2 ANTIGEN BINDING POLYPEPTIDES, POLYPEPTIDE COMPLEXES AND METHODS OF USE THEREOF

(71) Applicants: ModeX Therapeutics, Inc., Natick, MA (US); The United States of America, As Represented by the Secretary, Dept. of Health and Human Services, Bethesda, MD (US)

(72) Inventors: Juan LI, Boston, MA (US); Chi-Jen WEI, Weston, MA (US); Ronnie R. WEI, Weston, MA (US); Zhi-Yong YANG, Newton Center, MA (US); John R. MASCOLA, Rockville, MD (US); Gary J. NABEL, Delray Beach, FL (US); John MISASI, Bethesda, MD (US); Amarendra PEGU, Bethesda, MD (US); Lingshu WANG, Bethesda, MD (US); Tongqing ZHOU, Bethesda, MD (US); Misook CHOE, Bethesda, MD (US); Olamide K. OLONINIYI, Bethesda, MD (US); Bingchun ZHAO, Bethesda, MD (US); Yi ZHANG, Bethesda, MD (US); Eun Sung YANG, Bethesda, MD (US); Man CHEN, Bethesda, MD (US); Kwanyee LEUNG, Bethesda, MD (US); Wei SHI, Bethesda, MD (US); Nancy J. SULLIVAN, Bethesda, MD (US); Peter D. KWONG, Bethesda, MD (US); Richard A. KOUP, Bethesda, MD (US); Barney S. GRAHAM, Smyrna, GA (US); Peng HE, Natick, MA (US)

(21) Appl. No.: 18/345,478

(22) Filed: Jun. 30, 2023

Related U.S. Application Data
(60) Provisional application No. 63/357,331, filed on Jun. 30, 2022.
(60) Provisional application No. 63/381,842, filed on Nov. 1, 2022.
(60) Provisional application No. 63/481,368, filed on Jan. 24, 2023.
(60) Provisional application No. 63/357,336, filed on Jun. 30, 2022.
(60) Provisional application No. 63/357,873, filed on Jul. 1, 2022.
(60) Provisional application No. 63/381,850, filed on Nov. 1, 2022.
(60) Provisional application No. 63/404,473, filed on Sep. 7, 2022.
(60) Provisional application No. 63/433,719, filed on Dec. 19, 2022.

(51) Int. Cl. C07K 16/10; A61P 31/14; G01N 33/569

(52) U.S. Cl. CPC C07K 16/1003; A61P 31/14; G01N 33/56983; C07K 2317/24; C07K 2317/31; C07K 2317/522; C07K 2317/524; C07K 2317/526; C07K 2317/92

(57) ABSTRACT

Disclosed are antigen binding polypeptides and antigen binding polypeptide complexes (e.g., antibodies and antigen binding fragments thereof) having certain structural and/or functional features. Also disclosed are polynucleotides and vectors encoding such polypeptides and polypeptide complexes; host cells, pharmaceutical compositions and kits containing such polypeptides and polypeptide complexes; and methods of using such polypeptides and polypeptide complexes.

### CROSS REFERENCE TO RELATED APPLICATIONS

This application claims the priority benefit of U.S. Provisional Application No. 63/357,331, filed Jun. 30, 2022; U.S. Provisional Application No. 63/357,336, filed Jun. 30, 2022; U.S. Provisional Application No. 63/357,873, filed Jul. 1, 2022; U.S. Provisional Application No. 63/404,473, filed Sep. 7, 2022; U.S. Provisional Application No. 63/381,842, filed Nov. 1, 2022; U.S. Provisional Application No. 63/381,850, filed Nov. 1, 2022; U.S. Provisional Application No. 63/433,719, filed Dec. 9, 2022; and U.S. Provisional Application No. 63/481,368, filed Jan. 24, 2023, which are all incorporated herein by reference in their entireties.

### STATEMENT REGARDING FEDERALLY SPONSORED RESEARCH OR DEVELOPMENT

This invention was made in the performance of a Cooperative Research and Development Agreement with the National Institutes of Health, an Agency of the Department of Health and Human Services. The Government has certain rights in this invention.

### REFERENCE TO SEQUENCE LISTING SUBMITTED ELECTRONICALLY

The content of the electronically submitted sequence listing (Name: 4850_0110003_SEQLISTING_ST26.xml; Size: 1,589,914 bytes; Date of Creation: Jun. 29, 2023) is herein incorporated by reference in its entirety.

### FIELD

The present disclosure relates to anti-severe acute respiratory syndrome coronavirus 2 (anti-SARS-CoV-2) antigen binding polypeptides and polypeptide complexes (e.g., antibodies and antigen binding fragments thereof) having certain structural and/or functional features. The present disclosure also relates to polynucleotides and vectors encoding such polypeptides and polypeptide complexes; host cells; pharmaceutical compositions and kits containing such polypeptides and polypeptide complexes; and methods of using such polypeptides and polypeptide complexes.

### BACKGROUND

The outbreak of severe acute respiratory syndrome coronavirus (SARS-CoV-2) has caused over 450 million infections and over 6 million deaths worldwide. SARS-CoV-2 is the strain of coronavirus that causes COVID-19 (coronavirus disease 2019). Sequencing and publication of the original Wuhan Hu-1 (WA-1) genome enabled rapid vaccine design and production of spike proteins for therapeutic antibody development. However, persistent circulation of viruses during human pandemics leads to genetic variation that resists containment and eradication. While cellular and humoral immune responses are needed for optimal protection against viral infection, neutralizing antibodies play a major role in prevention and eradication of pathogens.

SARS-CoV-2 spike protein mutants and SARS-CoV-2 variants Alpha (B.1.1.7), Beta (B.1.351), Gamma (P.1), Delta (B.1.617.2), Epsilon variant (B.1.427), and Omicron (B.1.1.529) have emerged to contain mutations that are resistant to current therapies, have increased pathogenicity, and evade current vaccines. This led to their classification as variants of concern (VOCs).

Therapeutic antibodies have emerged as an important class of agents for treating human diseases and disorders. Therapeutic antibodies have been engineered to have specificity for two or more different antigens or epitopes (i.e., “multispecific” antibodies that are, for example, bispecific, trispecific or tetraspecific). Multispecific antibodies have been used to form multi-targeting strategies to treat human diseases and disorders, and are an important technological platform for developing neutralizing antibody-based therapeutics for preventing and treating SARS-CoV-2 infection and COVID-19.

There is a need for improved therapies to prevent and treat SARS-CoV-2 infection and COVID-19. There is a need for therapeutic antibodies with broad VOC reactivity and resistance to SARS-CoV-2 escape. There is also a need for therapeutic antibodies that are easier to manufacture and administer than current therapeutic antibody cocktails. There is also a need to treat or prevent SARS-CoV-2 infection and COVID-19 in immunocompromised individuals who cannot mount an effective immune response through vaccinations.

### BRIEF SUMMARY

Provided herein is a multispecific antibody selected from the group consisting of:
(a) an antigen binding polypeptide having a structure represented by:
   VL1-L1-VH1 or VH1-L2-VL1;
wherein:
   VL1 is a first immunoglobulin light chain variable region that specifically binds to a severe acute respiratory syndrome coronavirus 2 (SARS-CoV-2) protein;
   VH1 is a first immunoglobulin heavy chain variable region that specifically binds to a SARS-CoV-2 protein; and
   L1 and L2 are amino acid linkers; or
an antigen binding polypeptide having a structure represented by:
   VL1-L1-VL2-L2-VH2-L3-VH1;
   VL1-L1-VH2-L2-VL2-L3-VH1;
   VH1-L4-VH2-L5-VL2-L6-VL1; or
   VH1-L4-VL2-L5-VH2-L6-VL1;
wherein:
   VL1 is a first immunoglobulin light chain variable region that specifically binds to a SARS-CoV-2 protein;
   VL2 is a second immunoglobulin light chain variable region that specifically binds to a SARS-CoV-2 protein;
   VH1 is a first immunoglobulin heavy chain variable region that specifically binds to a SARS-CoV-2 protein;
   VH2 is a second immunoglobulin heavy chain variable region that specifically binds to a SARS-CoV-2 protein; and
   L1-L6 are amino acid linkers; and
   wherein (a) is selected from any one of the more specific embodiments provided herein for an antigen binding polypeptide in an antigen binding polypeptide complex having no more than three polypeptide chains;
(b) an antigen binding polypeptide complex comprising a first polypeptide, a second polypeptide, a third polypeptide, and a fourth polypeptide;
wherein the first polypeptide has a structure represented by:
   VL1-L1-CL;
wherein the second polypeptide has a structure represented by:
   VH1-L2-CH1;
wherein the third polypeptide has a structure represented by:
   VH2-L3-VH3-L4-CH1;
wherein the fourth polypeptide has a structure represented by:
   VL3-L5-VL2-L6-CL; and
wherein:
   VL1 is a first immunoglobulin light chain variable region that specifically binds to a SARS-CoV-2 protein;
   VL2 is a second immunoglobulin light chain variable region that specifically binds to a SARS-CoV-2 protein;
   VL3 is a third immunoglobulin light chain variable region that specifically binds to a SARS-CoV-2 protein;
   VH1 is a first immunoglobulin heavy chain variable region that specifically binds to a SARS-CoV-2 protein;
   VH2 is a second immunoglobulin heavy chain variable region that specifically binds to a SARS-CoV-2 protein;
   VH3 is a third immunoglobulin heavy chain variable region that specifically binds to a SARS-CoV-2 protein;
   CH1 is an immunoglobulin heavy chain constant region 1;
   CL is an immunoglobulin light chain constant region; and
   L1-L6 are amino acid linkers; and
(c) an antigen binding polypeptide having a structure represented by:
   VL1-L1-VH1 or VH1-L2-VL1;
wherein:
   VL1 is a first immunoglobulin light chain variable region that specifically binds to a SARS-CoV-2 protein;
   VH1 is a first immunoglobulin heavy chain variable region that specifically binds to a SARS-CoV-2 protein; and
   L1 and L2 are amino acid linkers; or
an antigen binding polypeptide having a structure represented by:
   VL1-L1-VL2-L2-VH2-L3-VH1;
   VL1-L1-VH2-L2-VL2-L3-VH1;
   VH1-L4-VH2-L5-VL2-L6-VL1; or
   VH1-L4-VL2-L5-VH2-L6-VL1;
wherein:
   VL1 is a first immunoglobulin light chain variable region that specifically binds to a SARS-CoV-2 protein;
   VL2 is a second immunoglobulin light chain variable region that specifically binds to a SARS-CoV-2 protein;
   VH1 is a first immunoglobulin heavy chain variable region that specifically binds to a SARS-CoV-2 protein;
   VH2 is a second immunoglobulin heavy chain variable region that specifically binds to a SARS-CoV-2 protein; and
   L1-L6 are amino acid linkers;
wherein (c) is selected from any one of the more specific embodiments provided herein for an antigen binding polypeptide in an antigen binding polypeptide complex having no more than three polypeptide chains; and
wherein the antigen binding polypeptide further comprises one or more immunoglobulin heavy chain constant region 1 (CH1) and/or one or more immunoglobulin light chain constant region (CL) between any one of the variable regions and/or amino acid linkers (e.g., between VL1 and L1, between L1 and VH1, between VH1 and L2, between L2 and VL1, between L1 and VL2, between VL2 and L2, between L2 and VH2, between VH2 and L3, between L3 and VH1, between VL1 and L1, between L1 and VH2, between VH2 and L2, between, L2 and VL2, between VL2 and L3, between L3 and VH1, between VH1 and L4, between L4 and VH2, between VH2 and L5, between L5 and VL2, between VL2 and L6, between L6 and VL1, between VH1 and L4, between L4 and VL2, between L4 and VL2, between VL2 and L5, between L5 and VH2, between VH2 and L6, or between L6 and VL1).

Also provided herein is a multispecific antibody comprising a first polypeptide, a second polypeptide, a third polypeptide, and a fourth polypeptide;
wherein the first polypeptide has a structure represented by VL1-L1-CL;
wherein the second polypeptide has a structure represented by VH1-L2-CH1-L3-Fc;
wherein the third polypeptide has a structure represented by VH2-L4-VH3-L5-CH1-L6-Fc;
wherein the fourth polypeptide has a structure represented by VL3-L7-VL2-L8-CL; and
wherein VL1 is a first immunoglobulin light chain variable region that specifically binds to a SARS-CoV-2 protein;
VL2 is a second immunoglobulin light chain variable region that specifically binds to a SARS-CoV-2 protein;
VL3 is a third immunoglobulin light chain variable region that specifically binds to a SARS-CoV-2 protein;
VH1 is a first immunoglobulin heavy chain variable region that specifically binds to a SARS-CoV-2 protein;
VH2 is a second immunoglobulin heavy chain variable region that specifically binds to a SARS-CoV-2 protein;
VH3 is a third immunoglobulin heavy chain variable region that specifically binds to a SARS-CoV-2 protein;
Fc is a region comprising an immunoglobulin heavy chain constant region 2 (CH2), an immunoglobulin heavy chain constant region 3 (CH3), and optionally, an immunoglobulin hinge;
CH1 is an immunoglobulin heavy chain constant region 1;
CL is an immunoglobulin light chain constant region; and
L1-L8 are amino acid linkers.

Provided herein is an antigen binding polypeptide having a structure represented by VL1-L1-VH1 or VH1-L2-VL1; wherein VL1 is a first immunoglobulin light chain variable region that specifically binds to a severe acute respiratory syndrome coronavirus 2 (SARS-CoV-2) protein; VH1 is a first immunoglobulin heavy chain variable region that specifically binds to a SARS-CoV-2 protein; and L1 and L2 are amino acid linkers.

Provided herein is an antigen binding polypeptide having a structure represented by VL1-L1-VL2-L2-VH2-L3-VH1; VL1-L1-VH2-L2-VL2-L3-VH1; VH1-L4-VH2-L5-VL2-L6-VL1; or VH1-L4-VL2-L5-VH2-L6-VL1; wherein VL1 is a first immunoglobulin light chain variable region that specifically binds to a SARS-CoV-2 protein; VL2 is a second immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; and L1-L6 are amino acid linkers.

Also provided herein is an antigen binding polypeptide having a structure represented by VL1-L1-VL2-L2-VH2-L3-VH1 or VH1-L4-VH2-L5-VL2-L6-VL1; wherein VL1 is a first immunoglobulin light chain variable region that specifically binds to a severe acute respiratory syndrome coronavirus 2 (SARS-CoV-2) protein; VL2 is a second immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; and L1-L6 are amino acid linkers.

Also provided herein is an antigen binding polypeptide having a structure represented by VL1-L1-VH2-L2-VL2-L3-VH1 or VH1-L4-VL2-L5-VH2-L6-VL1; wherein VL1 is a first immunoglobulin light chain variable region that specifically binds to a severe acute respiratory syndrome coronavirus 2 (SARS-CoV-2) protein; VL2 is a second immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; and L1-L6 are amino acid linkers.

Also provided herein is an antigen binding polypeptide complex comprising a first polypeptide and a second polypeptide; wherein the first polypeptide has a structure represented by VL1-L1-VL2-L2-VH2-L3-VH1; VL1-L1-VH2-L2-VL2-L3-VH1; VH1-L4-VH2-L5-VL2-L6-VL1; or VH1-L4-VL2-L5-VH2-L6-VL1; wherein the second polypeptide has a structure represented by VL3-L7-VL4-L8-VH4-L9-VH3; VL3-L7-VH4-L8-VL4-L9-VH3; VH3-L10-VH4-L11-VL4-L12-VL3; or VH3-L10-VL4-L11-VH4-L12-VL3; wherein VL1 is a first immunoglobulin light chain variable region that specifically binds to a SARS-CoV-2 protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; and L1-L12 are amino acid linkers.

Also provided herein is an antigen binding polypeptide complex comprising a first polypeptide and a second polypeptide; wherein the first polypeptide has a structure represented by VL1-L1-VH1 or VH1-L2-VL1; wherein the second polypeptide has a structure represented by VL2-L3-VH2 or VH2-L4-VL2; wherein VL1 is a first immunoglobulin light chain variable region that specifically binds to a SARS-CoV-2 protein; VH1 is a first immunoglobulin heavy chain variable region that specifically binds to a SARS-CoV-2 protein; VL2 is a second immunoglobulin light chain variable region that specifically binds to a SARS-CoV-2 protein; VH2 is a second immunoglobulin heavy chain variable region that specifically binds to a SARS-CoV-2 protein; and L1-L4 are amino acid linkers.

Also provided herein is an antigen binding polypeptide complex comprising a first polypeptide and a second polypeptide; wherein the first polypeptide has a structure represented by VL1-L1-VH1-L2-Fc or VH1-L3-VL1-L4-Fc; wherein the second polypeptide has a structure represented by VL2-L5-VH2-L6-Fc or VH2-L7-VL2-L8-Fc; wherein VL1 is a first immunoglobulin light chain variable region that specifically binds to a SARS-CoV-2 protein; VH1 is a first immunoglobulin heavy chain variable region that specifically binds to a SARS-CoV-2 protein; VL2 is a second immunoglobulin light chain variable region that specifically binds to a SARS-CoV-2 protein; VH2 is a second immunoglobulin heavy chain variable region that specifically binds to a SARS-CoV-2 protein; Fc is a region comprising an immunoglobulin heavy chain constant region 2 (CH2), an immunoglobulin heavy chain constant region 3 (CH3), and optionally, an immunoglobulin hinge; and L1-L8 are amino acid linkers.

Also provided herein is an antigen binding polypeptide complex comprising a first polypeptide and a second polypeptide; wherein the first polypeptide has a structure represented by VL1-L1-VL2-L2-VH2-L3-VH1 or VH1-L4-VH2-L5-VL2-L6-VL1; wherein the second polypeptide has a structure represented by VL3-L7-VL4-L8-VH4-L9-VH3 or VH3-L10-VH4-L11-VL4-L12-VL3; wherein VL1 is a first immunoglobulin light chain variable region that specifically binds to a SARS-CoV-2 protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; and L1-L12 are amino acid linkers.

Also provided herein is an antigen binding polypeptide complex comprising a first polypeptide and a second polypeptide; wherein the first polypeptide has a structure represented by VL1-L1-VH2-L2-VL2-L3-VH1 or VH1-L4-VL2-L5-VH2-L6-VL1; wherein the second polypeptide has a structure represented by VL3-L7-VH4-L8-VL4-L9-VH3 or VH3-L10-VL4-L11-VH4-L12-VL3; wherein VL1 is a first immunoglobulin light chain variable region that specifically binds to a SARS-CoV-2 protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; and L1-L12 are amino acid linkers.

Also provided herein is an antigen binding polypeptide having a structure represented by VL1-L1-VL2-L2-VH2-L3-VH1-L4-Fc; VL1-L1-VH2-L2-VL2-L3-VH1-L4-Fc; VH1-L5-VH2-L6-VL2-L7-VL1-L8-Fc; or VH1-L5-VL2-L6-VH2-L7-VL1-L8-Fc; wherein VL1 is a first immunoglobulin light chain variable region that specifically binds to a SARS-CoV-2 protein; VL2 is a second immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; Fc is a region comprising an immunoglobulin heavy chain constant region 2 (CH2), an immunoglobulin heavy chain constant region 3 (CH3), and optionally, an immunoglobulin hinge; and L1-L8 are amino acid linkers.

Also provided herein is an antigen binding polypeptide having a structure represented by VL1-L1-VL2-L2-VH2-L3-VH1-L4-Fc or VH1-L5-VH2-L6-VL2-L7-VL1-L8-Fc; wherein VL1 is a first immunoglobulin light chain variable region that specifically binds to a SARS-CoV-2 protein; VL2 is a second immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; Fc is a region comprising an immunoglobulin heavy chain constant region 2 (CH2), an immunoglobulin heavy chain constant region 3 (CH3), and optionally, an immunoglobulin hinge; and L1-L8 are amino acid linkers.

Also provided herein is an antigen binding polypeptide having a structure represented by VL1-L1-VH2-L2-VL2-L3-VH1-L4-Fc or VH1-L5-VL2-L6-VH2-L7-VL1-L8-Fc; wherein VL1 is a first immunoglobulin light chain variable region that specifically binds to a SARS-CoV-2 protein; VL2 is a second immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; Fc is a region comprising an immunoglobulin heavy chain constant region 2 (CH2), an immunoglobulin heavy chain constant region 3 (CH3), and optionally, an immunoglobulin hinge; and L1-L8 are amino acid linkers.

Also provided herein is an antigen binding polypeptide complex comprising a first polypeptide and a second polypeptide; wherein the first polypeptide has a structure represented by VL1-L1-VL2-L2-VH2-L3-VH1-L4-Fc; VL1-L1-VH2-L2-VL2-L3-VH1-L4-Fc; VH1-L5-VH2-L6-VL2-L7-VL1-L8-Fc; or VH1-L5-VL2-L6-VH2-L7-VL1-L8-Fc; wherein the second polypeptide has a structure represented by Fc; VL3-L9-VL4-L10-VH4-L11-VH3-L12-Fc; VL3-L9-VH4-L10-VL4-L11-VH3-L12-Fc; VH3-L13-VH4-L14-VL4-L15-VL3-L16-Fc; or VH3-L13-VL4-L14-VH4-L15-VL3-L16-Fc; wherein VL1 is a first immunoglobulin light chain variable region that specifically binds to a SARS-CoV-2 protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; Fc is a region comprising an immunoglobulin heavy chain constant region 2 (CH2), an immunoglobulin heavy chain constant region 3 (CH3), and optionally, an immunoglobulin hinge; and L1-L16 are amino acid linkers.

Also provided herein is an antigen binding polypeptide complex comprising a first polypeptide and a second polypeptide; wherein the first polypeptide has a structure represented by VL1-L1-VL2-L2-VH2-L3-VH1-L4-Fc or VH1-L5-VH2-L6-VL2-L7-VL1-L8-Fc; wherein the second polypeptide has a structure represented by Fc; VL3-L9-VL4-L10-VH4-L11-VH3-L12-Fc; or VH3-L13-VH4-L14-VL4-L15-VL3-L16-Fc; wherein VL1 is a first immunoglobulin light chain variable region that specifically binds to a SARS-CoV-2 protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; Fc is a region comprising an immunoglobulin heavy chain constant region 2 (CH2), an immunoglobulin heavy chain constant region 3 (CH3), and optionally, an immunoglobulin hinge; and L1-L16 are amino acid linkers.

Also provided herein is an antigen binding polypeptide complex comprising a first polypeptide and a second polypeptide; wherein the first polypeptide has a structure represented by VL1-L1-VH2-L2-VL2-L3-VH1-L4-Fc or VH1-L5-VL2-L6-VH2-L7-VL1-L8-Fc; wherein the second polypeptide has a structure represented by Fc; VL3-L9-VH4-L10-VL4-L11-VH3-L12-Fc or VH3-L13-VL4-L14-VH4-L15-VL3-L16-Fc; wherein VL1 is a first immunoglobulin light chain variable region that specifically binds to a SARS-CoV-2 protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; Fc is a region comprising an immunoglobulin heavy chain constant region 2 (CH2), an immunoglobulin heavy chain constant region 3 (CH3), and optionally, an immunoglobulin hinge; and L1-L16 are amino acid linkers.

Also provided herein is an antigen binding polypeptide having a structure represented by VL1-L1-VL2-L2-VH2-L3-VH1-L4-CH1-L5-CL; VL1-L1-VH2-L2-VL2-L3-VH1-L4-CH1-L5-CL; VH1-L6-VH2-L7-VL2-L8-VL1-L9-CH1-L10-CL; VH1-L6-VL2-L7-VH2-L8-VL1-L9-CH1-L10-CL; VL1-L11-VL2-L12-VH2-L13-VH1-L14-CL-L15-CH1; VL1-L11-VH2-L12-VL2-L13-VH1-L14-CL-L15-CH1; VH1-L16-VH2-L17-VL2-L18-VL1-L19-CL-L20-CH1; or VH1-L16-VL2-L17-VH2-L18-VL1-L19-CL-L20-CH1; wherein VL1 is a first immunoglobulin light chain variable region that specifically binds to a SARS-CoV-2 protein; VL2 is a second immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; CH1 is an immunoglobulin heavy chain constant region 1; CL is an immunoglobulin light chain constant region; and L1-L20 are amino acid linkers.

Also provided herein is an antigen binding polypeptide having a structure represented by VL1-L1-VL2-L2-VH2-L3-VH1-L4-CH1-L5-CL; VH1-L6-VH2-L7-VL2-L8-VL1-L9-CH1-L10-CL; VL1-L11-VL2-L12-VH2-L13-VH1-L14-CL-L15-CH1; or VH1-L16-VH2-L17-VL2-L18-VL1-L19-CL-L20-CH1; wherein VL1 is a first immunoglobulin light chain variable region that specifically binds to a SARS-CoV-2 protein; VL2 is a second immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; CH1 is an immunoglobulin heavy chain constant region 1; CL is an immunoglobulin light chain constant region; and L1-L20 are amino acid linkers.

Also provided herein is an antigen binding polypeptide having a structure represented by VL1-L1-VH2-L2-VL2-L3-VH1-L4-CH1-L5-CL; VH1-L6-VL2-L7-VH2-L8-VL1-L9-CH1-L10-CL; VL1-L11-VH2-L12-VL2-L13-VH1-L14-CL-L15-CH1; or VH1-L16-VL2-L17-VH2-L18-VL1-L19-CL-L20-CH1; wherein VL1 is a first immunoglobulin light chain variable region that specifically binds to a SARS-CoV-2 protein; VL2 is a second immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; CH1 is an immunoglobulin heavy chain constant region 1; CL is an immunoglobulin light chain constant region; and L1-L20 are amino acid linkers.

Also provided herein is an antigen binding polypeptide complex comprising a first polypeptide and a second polypeptide; wherein the first polypeptide has a structure represented by VL1-L1-VL2-L2-VH2-L3-VH1-L4-CH1-L5-CL; VL1-L1-VH2-L2-VL2-L3-VH1-L4-CH1-L5-CL; VH1-L6-VH2-L7-VL2-L8-VL1-L9-CH1-L10-CL; VH1-L6-VL2-L7-VH2-L8-VL1-L9-CH1-L10-CL; VL1-L11-VL2-L12-VH2-L13-VH1-L14-CL-L15-CH1; VL1-L11-VH2-L12-VL2-L13-VH1-L14-CL-L15-CH1; VH1-L16-VH2-L17-VL2-L18-VL1-L19-CL-L20-CH1; or VH1-L16-VL2-L17-VH2-L18-VL1-L19-CL-L20-CH1; wherein the second polypeptide has a structure represented by VL3-L21-VL4-L22-VH4-L23-VH3-L24-CH1-L25-CL; VL3-L21-VH4-L22-VL4-L23-VH3-L24-CH1-L25-CL; VH3-L26-VH4-L27-VL4-L28-VL3-L29-CH1-L30-CL; VH3-L26-VL4-L27-VH4-L28-VL3-L29-CH1-L30-CL; VL3-L31-VL4-L32-VH4-L33-VH3-L34-CL-L35-CH1; VL3-L31-VH4-L32-VL4-L33-VH3-L34-CL-L35-CH1; VH3-L36-VH4-L37-VL4-L38-VL3-L39-CL-L40-CH1; or VH3-L36-VL4-L37-VH4-L38-VL3-L39-CL-L40-CH1; wherein VL1 is a first immunoglobulin light chain variable region that specifically binds to a SARS-CoV-2 protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; CH1 is an immunoglobulin heavy chain constant region 1; CL is an immunoglobulin light chain constant region; and L1-L40 are amino acid linkers.

Also provided herein is an antigen binding polypeptide complex comprising a first polypeptide and a second polypeptide; wherein the first polypeptide has a structure represented by VL1-L1-VL2-L2-VH2-L3-VH1-L4-CH1-L5-CL; VH1-L6-VH2-L7-VL2-L8-VL1-L9-CH1-L10-CL; VL1-L11-VL2-L12-VH2-L13-VH1-L14-CL-L15-CH1; or VH1-L16-VH2-L17-VL2-L18-VL1-L19-CL-L20-CH1; wherein the second polypeptide has a structure represented by VL3-L21-VL4-L22-VH4-L23-VH3-L24-CH1-L25-CL; VH3-L26-VH4-L27-VL4-L28-VL3-L29-CH1-L30-CL; VL3-L31-VL4-L32-VH4-L33-VH3-L34-CL-L35-CH1; or VH3-L36-VH4-L37-VL4-L38-VL3-L39-CL-L40-CH1; wherein VL1 is a first immunoglobulin light chain variable region that specifically binds to a SARS-CoV-2 protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; CH1 is an immunoglobulin heavy chain constant region 1; CL is an immunoglobulin light chain constant region; and L1-L40 are amino acid linkers.

Also provided herein is an antigen binding polypeptide complex comprising a first polypeptide and a second polypeptide; wherein the first polypeptide has a structure represented by VL1-L1-VH2-L2-VL2-L3-VH1-L4-CH1-L5-CL; VH1-L6-VL2-L7-VH2-L8-VL1-L9-CH1-L10-CL; VL1-L11-VH2-L12-VL2-L13-VH1-L14-CL-L15-CH1; or VH1-L16-VL2-L17-VH2-L18-VL1-L19-CL-L20-CH1; wherein the second polypeptide has a structure represented by VL3-L21-VH4-L22-VL4-L23-VH3-L24-CH1-L25-CL; VH3-L26-VL4-L27-VH4-L28-VL3-L29-CH1-L30-CL; VL3-L31-VH4-L32-VL4-L33-VH3-L34-CL-L35-CH1; or VH3-L36-VL4-L37-VH4-L38-VL3-L39-CL-L40-CH1; wherein VL1 is a first immunoglobulin light chain variable region that specifically binds to a SARS-CoV-2 protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; CH1 is an immunoglobulin heavy chain constant region 1; CL is an immunoglobulin light chain constant region; and L1-L40 are amino acid linkers.

Also provided herein is an antigen binding polypeptide having a structure represented by VL1-L1-VL2-L2-VH2-L3-VH1-L4-CH1-L5-CL-L6-Fc; VL1-L1-VH2-L2-VL2-L3-VH1-L4-CH1-L5-CL-L6-Fc; VH1-L7-VH2-L8-VL2-L9-VL1-L10-CH1-L11-CL-L12-Fc; VH1-L7-VL2-L8-VH2-L9-VL1-L10-CH1-L11-CL-L12-Fc; VL1-L13-VL2-L14-VH2-L15-VH1-L16-CL-L17-CH1-L18-Fc; VL1-L13-VH2-L14-VL2-L15-VH1-L16-CL-L17-CH1-L18-Fc; VH1-L19-VH2-L20-VL2-L21-VL1-L22-CL-L23-CH1-L24-Fc; or VH1-L19-VL2-L20-VH2-L21-VL1-L22-CL-L23-CH1-L24-Fc; wherein VL1 is a first immunoglobulin light chain variable region that specifically binds to a SARS-CoV-2 protein; VL2 is a second immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; CH1 is an immunoglobulin heavy chain constant region 1; CL is an immunoglobulin light chain constant region; Fc is a region comprising an immunoglobulin heavy chain constant region 2 (CH2), an immunoglobulin heavy chain constant region 3 (CH3), and optionally, an immunoglobulin hinge; and L1-L24 are amino acid linkers.

Also provided herein is an antigen binding polypeptide having a structure represented by VL1-L1-VL2-L2-VH2-L3-VH1-L4-CH1-L5-CL-L6-Fc; VH1-L7-VH2-L8-VL2-L9-VL1-L10-CH1-L11-CL-L12-Fc; VL1-L13-VL2-L14-VH2-L15-VH1-L16-CL-L17-CH1-L18-Fc; or VH1-L19-VH2-L20-VL2-L21-VL1-L22-CL-L23-CH1-L24-Fc; wherein VL1 is a first immunoglobulin light chain variable region that specifically binds to a SARS-CoV-2 protein; VL2 is a second immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; CH1 is an immunoglobulin heavy chain constant region 1; CL is an immunoglobulin light chain constant region; Fc is a region comprising an immunoglobulin heavy chain constant region 2 (CH2), an immunoglobulin heavy chain constant region 3 (CH3), and optionally, an immunoglobulin hinge; and L1-L24 are amino acid linkers.

Also provided herein is an antigen binding polypeptide having a structure represented by VL1-L1-VH2-L2-VL2-L3-VH1-L4-CH1-L5-CL-L6-Fc; VH1-L7-VL2-L8-VH2-L9-VL1-L10-CH1-L11-CL-L12-Fc; VL1-L13-VH2-L14-VL2-L15-VH1-L16-CL-L17-CH1-L18-Fc; or VH1-L19-VH2-L20-VL2-L21-VL1-L22-CL-L23-CH1-L24-Fc; wherein VL1 is a first immunoglobulin light chain variable region that specifically binds to a SARS-CoV-2 protein; VL2 is a second immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; CH1 is an immunoglobulin heavy chain constant region 1; CL is an immunoglobulin light chain constant region; Fc is a region comprising an immunoglobulin heavy chain constant region 2 (CH2), an immunoglobulin heavy chain constant region 3 (CH3), and optionally, an immunoglobulin hinge; and L1-L24 are amino acid linkers.

Also provided herein is an antigen binding polypeptide complex comprising a first polypeptide and a second polypeptide; wherein the first polypeptide has a structure represented by VL1-L1-VL2-L2-VH2-L3-VH1-L4-CH1-L5-CL-L6-Fc; VL1-L1-VH2-L2-VL2-L3-VH1-L4-CH1-L5-CL-L6-Fc; VH1-L7-VH2-L8-VL2-L9-VL1-L10-CH1-L11-CL-L12-Fc; VH1-L7-VL2-L8-VH2-L9-VL1-L10-CH1-L11-CL-L12-Fc; VL1-L13-VL2-L14-VH2-L15-VH1-L16-CL-L17-CH1-L18-Fc; VL1-L13-VH2-L14-VL2-L15-VH1-L16-CL-L17-CH1-L18-Fc; VH1-L19-VH2-L20-VL2-L21-VL1-L22-CL-L23-CH1-L24-Fc; or VH1-L19-VL2-L20-VH2-L21-VL1-L22-CL-L23-CH1-L24-Fc; wherein the second polypeptide has a structure represented by Fc; VL3-L25-VL4-L26-VH4-L27-VH3-L28-CH1-L29-CL-L30-Fc; VL3-L25-VH4-L26-VL4-L27-VH3-L28-CH1-L29-CL-L30-Fc; VH3-L31-VH4-L32-VL4-L33-VL3-L34-CH1-L35-CL-L36-Fc; VH3-L31-VL4-L32-VH4-L33-VL3-L34-CH1-L35-CL-L36-Fc; VL3-L37-VL4-L38-VH4-L39-VH3-L40-CL-L41-CH1-L42-Fc; VL3-L37-VH4-L38-VL4-L39-VH3-L40-CL-L41-CH1-L42-Fc; VH3-L43-VH4-L44-VL4-L45-VL3-L46-CL-L47-CH1-L48-Fc; or VH3-L43-VL4-L44-VH4-L45-VL3-L46-CL-L47-CH1-L48-Fc; wherein VL1 is a first immunoglobulin light chain variable region that specifically binds to a SARS-CoV-2 protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; CH1 is an immunoglobulin heavy chain constant region 1; CL is an immunoglobulin light chain constant region; Fc is a region comprising an immunoglobulin heavy chain constant region 2 (CH2), an immunoglobulin heavy chain constant region 3 (CH3), and optionally, an immunoglobulin hinge; and L1-L48 are amino acid linkers.

Also provided herein is an antigen binding polypeptide complex comprising a first polypeptide and a second polypeptide; wherein the first polypeptide has a structure represented by VL1-L1-VL2-L2-VH2-L3-VH1-L4-CH1-L5-CL-L6-Fc; VH1-L7-VH2-L8-VL2-L9-VL1-L10-CH1-L11-CL-L12-Fc; VL1-L13-VL2-L14-VH2-L15-VH1-L16-CL-L17-CH1-L18-Fc; or VH1-L19-VH2-L20-VL2-L21-VL1-L22-CL-L23-CH1-L24-Fc; wherein the second polypeptide has a structure represented by Fc; VL3-L25-VL4-L26-VH4-L27-VH3-L28-CH1-L29-CL-L30-Fc; VH3-L31-VH4-L32-VL4-L33-VL3-L34-CH1-L35-CL-L36-Fc; VL3-L37-VL4-L38-VH4-L39-VH3-L40-CL-L41-CH1-L42-Fc; or VH3-L43-VH4-L44-VL4-L45-VL3-L46-CL-L47-CH1-L48-Fc; wherein VL1 is a first immunoglobulin light chain variable region that specifically binds to a SARS-CoV-2 protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; CH1 is an immunoglobulin heavy chain constant region 1; CL is an immunoglobulin light chain constant region; Fc is a region comprising an immunoglobulin heavy chain constant region 2 (CH2), an immunoglobulin heavy chain constant region 3 (CH3), and optionally, an immunoglobulin hinge; and L1-L48 are amino acid linkers.

Also provided herein is an antigen binding polypeptide complex comprising a first polypeptide and a second polypeptide; wherein the first polypeptide has a structure represented by VL1-L1-VH2-L2-VL2-L3-VH1-L4-CH1-L5-CL-L6-Fc; VH1-L7-VL2-L8-VH2-L9-VL1-L10-CH1-L11-CL-L12-Fc; VL1-L13-VH2-L14-VL2-L15-VH1-L16-CL-L17-CH1-L18-Fc; or VH1-L19-VL2-L20-VH2-L21-VL1-L22-CL-L23-CH1-L24-Fc; wherein the second polypeptide has a structure represented by Fc; VL3-L25-VH4-L26-VL4-L27-VH3-L28-CH1-L29-CL-L30-Fc; VH3-L31-VL4-L32-VH4-L33-VL3-L34-CH1-L35-CL-L36-Fc; VL3-L37-VH4-L38-VL4-L39-VH3-L40-CL-L41-CH1-L42-Fc; or VH3-L43-VL4-L44-VH4-L45-VL3-L46-CL-L47-CH1-L48-Fc; wherein VL1 is a first immunoglobulin light chain variable region that specifically binds to a SARS-CoV-2 protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; CH1 is an immunoglobulin heavy chain constant region 1; CL is an immunoglobulin light chain constant region; Fc is a region comprising an immunoglobulin heavy chain constant region 2 (CH2), an immunoglobulin heavy chain constant region 3 (CH3), and optionally, an immunoglobulin hinge; and L1-L48 are amino acid linkers.

Also provided herein is an antigen binding polypeptide or antigen binding polypeptide complex comprising a polypeptide having a structure represented by VL1-L1-VL2-L2-VH2-L3-VH1-L4-Fc-L5-Fc; VL1-L1-VH2-L2-VL2-L3-VH1-L4-Fc-L5-Fc; VH1-L6-VH2-L7-VL2-L8-VL1-L9-Fc-L10-Fc; or VH1-L6-VL2-L7-VH2-L8-VL1-L9-Fc-L10-Fc; wherein VL1 is a first immunoglobulin light chain variable region that specifically binds to a SARS-CoV-2 protein; VL2 is a second immunoglobulin light chain variable region that specifically binds a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; Fc is a region comprising an immunoglobulin heavy chain constant region 2 (CH2), an immunoglobulin heavy chain constant region 3 (CH3), and optionally, an immunoglobulin hinge; and L1-L10 are amino acid linkers.

Also provided herein is an antigen binding polypeptide or antigen binding polypeptide complex comprising a polypeptide having a structure represented by VL1-L1-VL2-L2-VH2-L3-VH1-L4-Fc-L5-Fc or VH1-L6-VH2-L7-VL2-L8-VL1-L9-Fc-L10-Fc; wherein VL1 is a first immunoglobulin light chain variable region that specifically binds to a SARS-CoV-2 protein; VL2 is a second immunoglobulin light chain variable region that specifically binds a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; Fc is a region comprising an immunoglobulin heavy chain constant region 2 (CH2), an immunoglobulin heavy chain constant region 3 (CH3), and optionally, an immunoglobulin hinge; and L1-L10 are amino acid linkers.

Also provided herein is an antigen binding polypeptide or antigen binding polypeptide complex comprising a polypeptide having a structure represented by VL1-L1-VH2-L2-VL2-L3-VH1-L4-Fc-L5-Fc; or VH1-L6-VL2-L7-VH2-L8-VL1-L9-Fc-L10-Fc; wherein VL1 is a first immunoglobulin light chain variable region that specifically binds to a SARS-CoV-2 protein; VL2 is a second immunoglobulin light chain variable region that specifically binds a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; Fc is a region comprising an immunoglobulin heavy chain constant region 2 (CH2), an immunoglobulin heavy chain constant region 3 (CH3), and optionally, an immunoglobulin hinge; and L1-L10 are amino acid linkers.

In some aspects, VH1, VH2, VH3 and VH4 each comprise the same heavy chain variable region, and VL1, VL2, VL3 and VL4 each comprise the same light chain variable region.

In some aspects, VH1 comprises the same heavy chain variable region as VH3, and VL1 comprises the same light chain variable region as VL3.

In some aspects, VH2 comprises the same heavy chain variable region as VH4, and VL2 comprises the same light chain variable region as VL4.

In some aspects, two of VH1, VH2, VH3 and VH4 comprise the same heavy chain variable region, and two of VL1, VL2, VL3 and VL4 comprise the same light chain variable region.

In some aspects, VH2 comprises the same heavy chain variable region as VH4, and VL2 comprises the same light chain variable region as VL4.

In some aspects, VH2 comprises the same heavy chain variable region as VH3, and VL2 comprises the same light chain variable region as VL3.

In some aspects, VH1 comprises the same heavy chain variable region as VH4, and VL1 comprises the same light chain variable region as VL4.

In some aspects, VH1 comprises the same heavy chain variable region as VH3, and VL1 comprises the same light chain variable region as VL3.

In some aspects, the immunoglobulin hinge comprises an upper hinge region, a middle hinge region, a lower hinge region, or a combination thereof.

In some aspects, linkers L1-L48 each independently have a length of from 0 amino acids to about 50 amino acids.

In some aspects, linkers L1-L48 not having a length of 0 amino acids each independently comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG.

In some aspects, one or more of linkers L1-L48 are non-immunogenic.

In some aspects, one or more of linkers L1-L48 do not contain a consensus T cell epitope.

In some aspects, the Fc region comprises at least one knob-into-hole modification or Fc effector function knockout mutation.

In some aspects, the antigen binding polypeptide complex is an IgG1 or IgG4 antibody and the knob-into-hole modification comprises (i) knob substitutions of S354C and T366W and hole substitutions of Y349C, T366S, L368A and Y407V; (ii) hole substitutions of M428L and N434S or N434A; (iii) hole substitutions of M252Y, S254T and T256E; or (iv) a combination thereof, based on the EU numbering scheme. In some aspects, the hole substitutions are M428L and N424S, based on the EU numbering scheme. In some aspects, the hole substitution is N434A, based on the EU numbering scheme.

In some aspects, the antigen binding polypeptide complex is an IgG1 or IgG4 antibody and the Fc effector function knockout mutation is L234A, L235A, P239A or a combination thereof, based on the EU numbering scheme.

In some aspects, the antigen binding polypeptide or antigen binding polypeptide complex comprises a detectable label. In some aspects, the detectable label is a radioactive label, chemiluminescent label, fluorescent label, enzyme, peptide tag, or a combination thereof. In some aspects, the peptide tag is a polyhistidine tag consisting of from about four to about 10 histidine residues. In some aspects, the polyhistidine tag consists of about eight histidine residues.

In some aspects, the antigen binding polypeptide or antigen binding polypeptide complex specifically binds to a SARS-CoV-2 protein with an equilibrium dissociation constant (K_{D}) of from about 10 μM to about 1 μM.

In some aspects, a variable region of an antibody may require site specific deglycosylation. In some aspects, and with respect to E8 VH mAb, an N62 mutation is made to remove a high mannose glycosylate residue or moiety. Preferred mutations include, but are not limited to, N62Q or N62S.

In some aspects, the antigen binding polypeptide complex is bispecific, trispecific or tetraspecific and has greater neutralization potency against SARS-CoV-2 virus than a monospecific antigen binding polypeptide complex that specifically binds to one of the same antigens the bispecific, trispecific or tetraspecific antigen binding polypeptide complex.

In some aspects, the antigen binding polypeptide complex is bispecific, trispecific or tetraspecific and has greater neutralization potency against SARS-CoV-2 virus than a mixture of monospecific antigen binding polypeptide complexes that specifically bind to the same antigens as the bispecific, trispecific or tetraspecific antigen binding polypeptide complex.

In some aspects, the SARS-CoV-2 virus is the original Wuhan strain (WA1), D614G spike protein mutant (D614G), Alpha variant (B.1.1.7), Beta variant (B.1.351), Gamma variant (P.1), Delta variant, Epsilon variant (B.1.427), or Omicron variant (B.1.1.529), or a combination thereof. In some aspects, the Omicron variant is BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, CH.1.1, or a combination thereof.

In some aspects, the SARS-CoV-2 protein is a membrane protein, nucleocapsid protein, envelope protein, or spike protein. In some aspects, the SARS-CoV-2 protein is a spike protein.

Also provided herein is an antibody or antigen binding fragment thereof comprising an antigen binding polypeptide or antigen binding polypeptide complex provided herein.

In some aspects, the antibody is IgG, IgM, IgE, IgA or IgD. In some aspects, the IgG is IgG1, IgG2, IgG3 or IgG4.

In some aspects, the antigen binding fragment is a Fab, scFab, Fab′, F(ab′)₂, Fv or scFv.

In some aspects, the antibody is human or humanized.

In some aspects, the antibody is a monoclonal antibody, grafted antibody, or chimeric antibody.

Also provided herein is a pharmaceutical composition comprising an antigen binding polypeptide, antigen binding polypeptide complex, antibody or antigen binding fragment thereof provided herein. In some aspects, the pharmaceutical composition comprises (1) one or more polynucleotides encoding an antigen binding polypeptide or antigen binding polypeptide complex, and (2) a carrier. In some aspects, the pharmaceutical composition comprises (1) one or more polynucleotides encoding an antigen binding polypeptide or antigen binding polypeptide complex (e.g., antibody or antigen binding fragment thereof) provided herein, and (2) a carrier selected from the group consisting of a lipid or a lipid nanoparticle. In some aspects, the one or more polynucleotides encoding an antigen binding polypeptide or antigen binding polypeptide complex is a stabilized polynucleotide resistant to degradation or decomposition in vivo. In some aspects, the stabilized polynucleotide is capped at one end (5′ cap) and has a long polyadenylated (polyA) tail at the other end to form a stabilized mRNA having 5′ and 3′ UTRs. In some aspects, the polyA tail is about 50 bp, about 100 bp, about 120 bp, or about 150 bp. In some aspects, modified nucleosides may be incorporated into the mRNA to increase translation and/or to lower potential immunogenicity.

Also provided herein is a pharmaceutical composition comprising an antigen binding polypeptide, antigen binding polypeptide complex, antibody, or antigen binding fragment thereof provided herein, and an additional pharmaceutical agent.

Also provided herein is a kit comprising an antigen binding polypeptide, antigen binding polypeptide complex, antibody or antigen binding fragment thereof, or a pharmaceutical composition provided herein.

Also provided herein is a method of preventing or treating a SARS-CoV-2 viral infection in a subject in need thereof, comprising administering to the subject a therapeutically effective amount of an antigen binding polypeptide or polypeptide complex provided herein, an antibody or antigen binding fragment thereof provided herein, a pharmaceutical composition provided herein, or a combination thereof. Delivery of the therapeutic antigen binding polypeptide or antigen binding polypeptide complex can be by direct administration of the therapeutic or by an indirect method that is inclusive of a modified mRNA composition that is administered to the patient in need of treatment thereof by a therapeutic antigen binding polypeptide or antigen binding polypeptide complex translated from the modified mRNA that codes for the therapeutic polypeptide or polypeptide complex.

Also provided herein is a method of preventing or treating coronavirus disease 2019 (COVID-19) in a subject in need thereof, comprising administering to the subject a therapeutically effective amount of an antigen binding polypeptide or polypeptide complex provided herein, an antibody or antigen binding fragment thereof provided herein, a pharmaceutical composition provided herein, or a combination thereof.

Also provided herein is a method of diagnosing a subject as being infected with a SARS-CoV-2 virus or suspected of being infected with a SARS-CoV-2 virus, comprising (i) contacting a sample obtained from the subject with an antigen binding polypeptide or polypeptide complex provided herein or an antibody or antigen binding fragment thereof provided herein; (ii) detecting the presence or absence of a virus complex which contains the polypeptide, polypeptide complex, antibody or fragment and a SARS-CoV-2 virus, virion or fragment thereof; and (iii) diagnosing the subject as being infected with a SARS-CoV-2 virus or suspected of being infected with a SARS-CoV-2 virus when the presence of the virus complex is detected.

Also provided herein is a method of diagnosing a subject as not being infected with a SARS-CoV-2 virus or not suspected of being infected with a SARS-CoV-2 virus, comprising (i) contacting a sample obtained from the subject with an antigen binding polypeptide or polypeptide complex provided herein or an antibody or antigen binding fragment thereof provided herein; (ii) detecting the presence or absence of a virus complex which contains the polypeptide, polypeptide complex, antibody or fragment and a SARS-CoV-2 virus, virion or fragment thereof, and (iii) diagnosing the subject as not being infected with a SARS-CoV-2 virus or not suspected of being infected with a SARS-CoV-2 virus when the presence of the virus complex is not detected.

Also provided herein is a method of diagnosing a subject as having COVID-19 or suspected of having COVID-19, comprising (i) contacting a sample obtained from the subject with an antigen binding polypeptide or polypeptide complex provided herein or an antibody or antigen binding fragment thereof provided herein; (ii) detecting the presence or absence of a virus complex which contains the polypeptide, polypeptide complex, antibody or fragment and a SARS-CoV-2 virus, virion or fragment thereof, and (iii) diagnosing the subject as having COVID-19 or suspected of having COVID-19 when the presence of the virus complex is detected.

Also provided herein is a method of diagnosing a subject as not having COVID-19 or not suspected of having COVID-19, comprising (i) contacting a sample obtained from the subject with an antigen binding polypeptide or polypeptide complex provided herein or an antibody or antigen binding fragment thereof provided herein; (ii) detecting the presence or absence of a virus complex which contains the polypeptide, polypeptide complex, antibody or fragment and a SARS-CoV-2 virus, virion or fragment thereof, and (iii) diagnosing the subject as not having COVID-19 or not suspected of having COVID-19 when the presence of the virus complex is not detected.

In some aspects, the sample is a nasal swab, tissue sample, saliva, plasma or blood.

In some aspects, detecting the presence or absence of the virus complex comprises an enzyme linked immunosorbent assay (ELISA), immunospot assay, lateral flow assay, flow cytometry, immunohistochemistry or western blot.

In some aspects, the polypeptide, polypeptide complex, antibody or fragment is bispecific, trispecific or tetraspecific and has greater neutralization potency against SARS-CoV-2 virus than a monospecific polypeptide, polypeptide complex, antibody or fragment that specifically binds to one of the same antigens the bispecific, trispecific or tetraspecific polypeptide, polypeptide complex, antibody or fragment.

In some aspects, the polypeptide, polypeptide complex, antibody or fragment is bispecific, trispecific or tetraspecific and has greater neutralization potency against SARS-CoV-2 virus than a mixture of monospecific polypeptides, polypeptide complexes, antibodies or fragments that specifically bind to the same antigens as the bispecific, trispecific or tetraspecific polypeptide, polypeptide complex, antibody or fragment.

In some aspects, the SARS-CoV-2 virus is the original Wuhan strain (WA1), D614G spike protein mutant (D614G), Alpha variant (B.1.1.7), Beta variant (B.1.351), Gamma variant (P.1), Delta variant, Epsilon variant (B.1.427), Omicron variant (B.1.1.529), or a combination thereof. In some aspects, the Omicron variant is BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, CH.1.1, or a combination thereof.

Also provided herein is a method of preventing immune escape of a SARS-CoV2 virus in a subject infected with the SARS-CoV2 virus, comprising administering to the subject an effective amount of an antigen binding polypeptide or polypeptide complex provided herein, an antibody or antigen binding fragment thereof provided herein, a pharmaceutical composition provided herein, or a combination thereof.

Also provided herein is a method of preventing immune escape of a SARS-CoV2 virus in a subject infected with the SARS-CoV2 virus, comprising administering to the subject an effective amount of an anti-SARS-CoV2 multivalent antibody that exhibits improved prevention of immune escape compared to (i) a monovalent anti-SARS-CoV2 antibody that binds to one of the same antigens as the anti-SARS-CoV2 multivalent antibody, and/or (ii) a combination of monovalent anti-SARS-CoV2 antibodies that binds to the same antigens as the anti-SARS-CoV2 multivalent antibody.

Also provided herein is a method of neutralizing a SARS-CoV-2 virus in a subject infected with the SARS-CoV2 virus, comprising administering to the subject an effective amount of an antigen binding polypeptide or polypeptide complex provided herein, an antibody or antigen binding fragment thereof provided herein, or a combination thereof.

Also provided herein is a method of increasing neutralization potency against a SARS-CoV-2 virus in a subject infected with the SARS-CoV2 virus, comprising administering to the subject an effective amount of an antigen binding polypeptide or polypeptide complex provided herein, an antibody or antigen binding fragment thereof provided herein, or a combination thereof.

Also provided herein is a method of increasing neutralization potency against a SARS-CoV-2 virus in a subject infected with the SARS-CoV2 virus, comprising administering to the subject an effective amount of an anti-SARS-CoV2 multivalent antibody that exhibits increased neutralization potency compared to (i) a monovalent anti-SARS-CoV2 antibody that binds to one of the same antigens as the anti-SARS-CoV2 multivalent antibody and/or (ii) a combination of monovalent anti-SARS-CoV2 antibodies that binds to the same antigens as the anti-SARS-CoV2 multivalent antibody.

Also provided herein is an anti-SARS-CoV2 multivalent antibody that exhibits improved prevention of immune escape compared to (i) a monovalent anti-SARS-CoV2 antibody that binds to one of the same antigens as the anti-SARS-CoV2 multivalent antibody, and/or (ii) a combination of monovalent anti-SARS-CoV2 antibodies that binds to the same antigens as the anti-SARS-CoV2 multivalent antibody.

### BRIEF DESCRIPTION OF THE DRAWINGS

Some aspects of the invention are herein described, by way of example only, with reference to the accompanying drawings. With specific reference now to the drawings in detail, it is stressed that the particulars shown are by way of example and for purposes of illustrative discussion of aspects of the invention.

FIG. 1A-1D shows configurations of exemplary monospecific, tetravalent molecules described in the Examples. FIG. 1A: monospecific, tetravalent molecule containing VH and VL of the anti-SARS-CoV-2 antibody, B1-182, with Fc region (MX241). FIG. 1B: monospecific, tetravalent molecule containing VH and VL of B1-182, with Fc, CL and CH1 (MX365). FIG. 1C: monospecific, tetravalent molecule containing VH and VL of the anti-SARS-CoV-2 hybrid antibody, A23-58H/B1-182L, with Fc (MX409). FIG. 1D: monospecific, tetravalent molecule containing VH and VL of A23-58H/B1-182L, with Fc, CL and CH1 (MX466).

FIG. 2A-2P shows configurations of exemplary bispecific, tetravalent molecules described in the Examples. FIG. 2A: bispecific, tetravalent molecule containing VH and VL of B1-182 and VH and VL of the anti-SARS-CoV-2 antibody, A19-46, with Fc (MX179). FIG. 2B: bispecific, tetravalent molecule containing VH and VL of B1-182 and VH and VL of A19-46, with Fc (MX180). FIG. 2C: bispecific, tetravalent molecule containing VH and VL of B1-182 and VH and VL of the anti-SARS-CoV-2 antibody, A19-61, with Fc (MX183). FIG. 2D: bispecific, tetravalent molecule containing VH and VL of B1-182 and VH and VL of A19-61, with Fc (MX184). FIG. 2E: bispecific, tetravalent molecule containing VH and VL of B1-182 and VH and VL of A19-61, with Fc, CL and CH1 (MX366). FIG. 2F: bispecific, tetravalent molecule containing VH and VL of B1-182 and VH and VL of A19-61, with Fc, CL and CH1 (MX367). FIG. 2G: bispecific, tetravalent molecule containing VH and VL of A23-58H/B1-182L and VH and VL of A19-61, with Fc (MX412). FIG. 2H: bispecific, tetravalent molecule containing VH and VL of A23-58H/B1-182L and VH and VL of A19-61, with Fc (MX413). FIG. 2I: bispecific, tetravalent molecule containing VH and VL of A23-58H/B1-182L and VH and VL of A19-46, with Fc (MX490). FIG. 2J: bispecific, tetravalent molecule containing VH and VL of A23-58H/B1-182L and VH and VL of A19-46, with Fc (MX492). FIG. 2K: bispecific, tetravalent molecule containing VH and VL of A23-58H/B1-182L and VH and VL of A19-61, with Fc, CL and CH1 (MX467). FIG. 2L: bispecific, tetravalent molecule containing VH and VL of A23-58H/B1-182L and VH and VL of A19-61, with Fc, CL and CH1 (MX468). FIG. 2M: bispecific, tetravalent molecule containing VH and VL of A23-58H/B1-182L and VH and VL of A19-46, with Fc, CL and CH1 (MX491). FIG. 2N: bispecific, tetravalent molecule containing VH and VL of A23-58H/B1-182L and VH and VL of A19-46, with Fc, CL and CH1 (MX493). FIG. 2O: bispecific, tetravalent molecule containing VH and VL of the anti-SARS-CoV-2 antibody, A23-58, and VH and VL of A19-61 (MX326). FIG. 2P: bispecific, tetravalent molecule containing VH and VL of A23-58 and VH and VL of A19-61 (MX327).

FIG. 3A-3H shows exemplary configurations of trispecific, tetravalent molecules described in the Examples. FIG. 3A: trispecific, tetravalent molecule containing VH and VL of A19-46, VH and VL of A19-61, and VH and VL of B1-182 with Fc and knob-into-hole modification (MX494). FIG. 3B: trispecific, tetravalent molecule containing VH and VL of A19-46, VH and VL of A19-61, and VH and VL of B1-182 with Fc and knob-into-hole modification (MX495). FIG. 3C: trispecific, tetravalent molecule containing VH and VL of A19-46, VH and VL of A19-61, and VH and VL of B1-182 with Fc and knob-into-hole modification (MX496). FIG. 3D: trispecific, tetravalent molecule containing VH and VL of A19-46, VH and VL of A19-61, and VH and VL of B1-182 with Fc and knob-into-hole modification (MX497). FIG. 3E: trispecific, tetravalent molecule containing VH and VL of A19-46, VH and VL of A19-61, and VH and VL of A23-58H/B1-182L with Fc and knob-into-hole modification (MX498). FIG. 3F: trispecific, tetravalent molecule containing VH and VL of A19-46, VH and VL of A19-61, and VH and VL of A23-58H/B1-182L with Fc and knob-into-hole modification (MX499). FIG. 3G: trispecific, tetravalent molecule containing VH and VL of A19-46, VH and VL of A19-61, and VH and VL of A23-58H/B1-182L with Fc and knob-into-hole modification (MX500). FIG. 3H: trispecific, tetravalent molecule containing VH and VL of A19-46, VH and VL of A19-61, and VH and VL of A23-58H/B1-182L with Fc and knob-into-hole modification (MX501).

FIG. 4 shows the percent neutralization of Omicron variant (B.1.1.529) in the presence of increasing concentration of MX179 and MX180 antibodies described in Example 1, along with B1-182 and A19-46 parental antibodies.

FIG. 5 shows the percent neutralization of Omicron variant (B.1.1.529) in the presence of increasing concentration of MX179 or MX180 antibodies described in Example 1, in comparison to the combination of B1-182 and A19-46 parental antibodies. Concentration of each antibody in the combination of two antibody mixture was plotted.

FIG. 6 shows the percent neutralization of Omicron variant (B.1.1.529) in the presence of increasing concentration of MX494, MX495, MX496 or MX497 antibodies described in Example 1, in comparison to the combination of B1-182, A19-46 and A19-61 parental antibodies. Concentration of each antibody in the combination of three antibody mixture was plotted.

FIG. 7 shows serum antibody levels in Tg32-humanized FcRn mice (n=5) treated with parental or tetravalent bispecific MX413 (A19-61×A23-58H/B1-182L) antibody. Concentrations of VRC01 LS (dashed line, circle symbol), B1-182.1 (dotted line, square symbol), and MX413 (solid line, triangle symbol) antibodies containing a Fc mutation to extend half-life were measured in serum over the course of 35 days after intravenous administration of a single 5 mg/kg dose of each antibody. Each data point represents the mean±standard error of the mean (SEM) determined in triplicates.

FIG. 8A shows a diagram of the in vivo passive transfer challenge study described in Example 4.

FIGS. 8B-8D show the results from BA.1 challenge. FIG. 8B shows the weight loss of animals. Symbols and error bars represent means and SEM, respectively. FIG. 8C shows the lung viral load of animals. FIG. 8D shows the nares viral load of animals. Each symbol represents an individual animal and may overlap for equivalent values. An unpaired, two-tailed student t-test was used to determine significance at each timepoint between treated and untreated animals. Significant p-values are indicated as: * (≤0.05), † (≤0.01), ‡ (≤0.001), and ¥ (0.0001). Boxes and horizontal bars denote the infected subjects median (IQR) and medians, respectively. Whisker end points are equal to the maximum and minimum values. Dotted lines indicate the assay's lower limit of detection. An unpaired, two-tailed student t-test was used to determine significance at each timepoint between treated and untreated animals. Significant p-values are indicated on graphs.

FIGS. 8E-8G show the results from BA.2 challenge. FIG. 8E shows weight loss of animals. Symbols and error bars represent means and SEM, respectively. FIG. 8F shows the lung viral load of animals. FIG. 8G shows the nares viral load of animals. Each symbol represents an individual animal and may overlap for equivalent values. An unpaired, two-tailed student t-test was used to determine significance at each timepoint between treated and untreated animals. Significant p-values are indicated as: * (≤0.05), † (≤0.01), ‡ (≤0.001), and ¥ (≤0.0001). Boxes and horizontal bars denote the IQR and medians, respectively. Whisker end points are equal to the maximum and minimum values. Dotted lines indicate the assay's lower limit of detection. An unpaired, two-tailed student t-test was used to determine significance at each timepoint between treated and untreated animals. Significant p-values are indicated on graphs.

FIGS. 8H-8J show the results from BA.5 challenge. FIG. 8H shows weight loss of animals. Symbols and error bars represent means and SEM, respectively. FIG. 8I shows the lung viral load of animals. FIG. 8J shows the nares viral load of animals. Each symbol represents an individual animal and may overlap for equivalent values. An unpaired, two-tailed student t-test was used to determine significance at each timepoint between treated and untreated animals. Significant p-values are indicated as: * (≤0.05), † (≤0.01), ‡ (≤0.001), and ¥ (≤0.0001). Boxes and horizontal bars denote the IQR and medians, respectively. Whisker end points are equal to the maximum and minimum values. Dotted lines indicate the assay's lower limit of detection. An unpaired, two-tailed student t-test was used to determine significance at each timepoint between treated and untreated animals. Significant p-values are indicated on graphs.

FIG. 9 shows two configurations of trispecific, trivalent cross-over dual variable (CODV) antibodies made utilizing variable fragments (Fv) from B1-182.1 (“182.1”, dotted; light chain=lighter shading; heavy chain=darker shading), the anti-SARS-CoV-2 antibody, A19-46.1 (“46.1”, striped; light chain=lighter shading; heavy chain=darker shading) and A19-61.1 (“61.1”, grid; light chain=lighter shading; heavy chain=darker shading). CH1, CL and Fc regions are also indicated, along with a knob-into-hole modification in the Fc region.

FIG. 10 shows SARS-CoV-2 escape potential by monospecific antibodies (B1-182.1), a combination of monospecific antibodies (B1-182.1+A19-61.1+A19-46.1), and multispecific antibodies (46.1/61.1-182.1 and 61.1/46.1-182.1). Shown for each antibody is the maximum concentration with >20% cytopathic effect (CPE) that was passaged forward in each round of selection.

FIG. 11 shows exemplary configurations of tetravalent, bispecific and tetravalent, trispecific molecules. MX828 contains two VH/VL pairs of A19-46 and two VH/VL pairs of A23-58H/A23-58L93VGLTG. MX829 contains two VH/VL pairs of A19-61 and two VH/VL pairs of A23-58H/A23-58L93VGLTG. MX830 contains two VH/VL pairs of A19-46 and two VH/VL pairs of A23-58H/A23-58L94GLTG. MX831 contains two VH/VL pairs of A19-61 and two VH/VL pairs of A23-58H/A23-58L94GLTG. MX834 and MX837 contain one VH/VL pair of A19-46, one VH/VL pair of A19-61, and two VH/VL pairs of A23-58H/A23-58L93VGLTG. MX835 and MX838 contain one VH/VL pair of A19-46, one VH/VL pair of A19-61, and two VH/VL pairs of A23-58H/A23-58L94GLTG.

FIG. 12A-12F shows additional exemplary configurations of bivalent and tetravalent molecules.

FIG. 13A-13F shows additional exemplary configurations of tetravalent molecules having VH/VL binding pairs selected from B8, E8 and A19-46.

FIG. 14A-14L shows additional exemplary configurations of tetravalent molecules having VH/VL binding pairs selected from B8, A19-46 and E8.

FIG. 15A-15F shows additional exemplary configurations of tetravalent molecules having VH/VL binding pairs selected from E12, A19-46 and E8.

FIG. 16A-16L shows additional exemplary configurations of tetravalent molecules having VH/VL binding pairs selected from E12, A19-46 and E8.

FIG. 17A-17E shows additional exemplary configurations of tetravalent molecules having VH/VL binding pairs selected from B8, E12, A23-58H/L93VGLTG, A19-46 and E8.

FIG. 18A-18L shows additional exemplary configurations of tetravalent molecules having VH/VL binding pairs selected from A23-58H/L93VGLTG, A19-46 and E8.

FIG. 19 shows the IC50 values (in nM) of the indicated molecules for neutralization of Omicron sublineages BA.1, BA.4/5, BA.2.12.1, BA.2.75, BA.4.6, BA.2.75.2, BQ.1.1, BJ.1 and XBB.

FIG. 20 shows the IC50 values (in nM) of the indicated molecules for neutralization of Omicron sublineages BA.4/5, BQ.1, BQ.1.1, BA.2.75.2 and XBB.

FIG. 21 shows the IC50 values (in nM) of the indicated molecules for neutralization of Omicron sublineages BA.4/5, BQ.1, BQ.1.1, BA.2.75.2 and XBB.

FIG. 22 shows the IC50 and IC80 values (in nM) of the indicated molecules for neutralization of Omicron sublineages BA.4/5, BQ.1, BQ.1.1, BA.2.75.2, XBB, XBB1.5, BF.7 and CH.1.1. Class designations are shown in column 2 in the table

FIG. 23 shows an epitope map of Omicron, indicating various mutations and variants.

FIG. 24 shows the blood levels of MX1069, MX1043, MX1206, MX1089 and MX1042 in Syrian hamsters up to 21 days after infusion.

FIG. 25 shows a diagram of a passive transfer experiment performed in Syrian hamsters using MX1069 and MX1089 and XBB virus.

FIG. 26A shows the percent body weight change of XBB-infected animals treated with MX1069 and MX1089.

FIG. 26B shows the lung viral load (left panel) and nares viral load (right panel) of XXB-infected animals treated with MX1069 and MX1089.

FIG. 27 shows a diagram of a passive transfer experiment performed in Syrian hamsters, using MX1042 and MX1043 and XBB virus.

FIG. 28A shows the percent body weight change of XBB-infected animals treated with MX1042, MX1043 and a combination of MX1042 and MX1043.

FIG. 28B shows the lung viral load (left panel) and nares viral load (right panel) of XXB-infected animals treated with MX1042, MX1043 and a combination of MX1042 and MX1043.

FIG. 29A shows the size exclusion chromatography (SEC) elution profile of 46.1-182.1v/61.1-182.1v for preparative and analytic SEC.

FIG. 29B shows the IC50 and IC80 values (in pM) of the indicated molecules, for neutralization of Omicron sublineages B.1.1.7, BA.1, BA.2 and BA.4/5.

FIG. 30A shows lung histopathology representative images of BA.5-infected Syrian hamsters treated with PBS or 46.1-182.1v/61.1-182.1v at Day 2 and Day 4.

FIG. 30B shows lung histopathology representative images of BA.5-infected Syrian hamsters treated with PBS or 46.1-182.1v/61.1-182.1v at Day 6 and Day 10.

FIG. 31 shows SARS-CoV-2 escape potential by multispecific antibodies MX1043, MX1042, MX1069 and MX1089, compared to combinations of antibodies (E12+A19-46.1+E8; E12+E8; and MX1042+MX1043) and LY1404. Shown for each antibody is the maximum concentration with >20% CPE that was passaged forward in each round of selection. Structures for the relevant antibodies and the resulting IC50 and IC80 values are also shown.

FIG. 32 shows the results of a pharmacokinetic study to assess the properties of parental monoclonal antibodies F769-E12, F769-B1, E76-B8 and F770-E8, compared to bispecific antibodies MX1042 (E8×E12) and MX1043 (E12×A19-46); trispecific antibody MX1069 (A19-46×E12/E12×E8); and tetraspecific antibodies MX1089 (A19-46×B8/E8×A23-58H/L93VGLTG) and MX1206 (A19-46×E12/E8×A23-58H/L93VGLTL).

FIG. 33 shows the characterization of mRNA-LNP described in the examples, including the average diameter (nm) and polydispersity index (PDI).

FIG. 34 shows a diagram of the mouse model used to evaluate in vivo expression of mRNA-LNPs.

FIG. 35 shows blood antibody concentration in g/mL of mice treated with the indicated mRNA-LNP.

FIG. 36 shows an exemplary configuration of a monospecific, trivalent cross-over dual variable (CODV) immunoglobulin antibody made utilizing variable fragments (Fv) from the anti-SARS-CoV-2 antibody, B1-182.1 (Fv182.1, dotted). The Fc region contains a knob-into-hole modification to increase yield and correct association of heavy chains.

FIG. 37A-37C shows exemplary configurations of bispecific, trivalent CODV immunoglobulin antibodies made utilizing variable fragments (Fv) from B1-182.1 (Fv182.1, dotted) and the anti-SARS-CoV-2 antibody, A19-61.1 (Fv61.1, grid). The Fc region contains a knob-into-hole modification to increase yield and correct association of heavy chains.

FIG. 38A-38D shows exemplary configurations of trispecific, trivalent CODV immunoglobulin antibodies made utilizing variable fragments (Fv) from B1-182.1 (“182.1” or “Fv182 Class I”, dotted), the anti-SARS-CoV-2 antibody, A19-46.1 (“46.1” or “Fv46 Class II”, striped) and A19-61.1 (“61.1” or “Fv61 Class III”, grid). The Fc region contains a knob-into-hole modification to increase yield and correct association of heavy chains.

FIG. 39A shows purity of CODV immunoglobulin antibodies evaluated under non-reducing (nr) and reducing (r) conditions on a Coomassie SDS-PAGE gel (representative gels shown). A molecular weight marker standard (MW) is also shown.

FIG. 39B shows CODV immunoglobulin bispecific and trispecific antibody traces shown before (top row) and after size exclusion chromatography (SEC) (bottom row). The dotted box indicates the fractions combined to make final preparations of the indicated multispecific antibodies. The bottom row shows analytic SEC traces for the purified multispecific antibodies.

FIG. 40A shows cross-over dual variable immunoglobulin antibody designs used in FIGS. 40B-40E.

FIG. 40B shows the expected binding of Fv182.1 (“Fv182”), Fv46.1 (“Fv46”), and Fv61.1 (“Fv61”) to viral spike receptor-binding domain (RBD) proteins containing the indicated Fv isolating mutations, that were selected to knockout binding a single component Fv while leaving binding of the remain Fv components unchanged.

FIG. 40C-40E shows validation of multispecific Fv binding activity using Fv isolating mutations in RBD and enzyme-linked immunosorbent assay (ELISA). Shown is ELISA binding for parental component antibody controls (FIG. 40C, B1-182.1, A19-46.1, and A19-61.1), bispecific antibodies (FIG. 40D, 182.1/182.1-61.1, 182.1/61.1-182.1, 61.1/182.1-182.1, 61.1/61.1-182.1 and 61.1/182.1-61.1), and trispecific antibodies (FIG. 40E, 46.1/61.1-182.1, 46.1/182.1-61.1, 61.1/46.1-182.1 and 61.1/182.1-46.1) against WA-1 RBD (wt) or RBD proteins containing one or more Fv-specific mutation shown and chosen to interrogate binding of one Fv component at a time in each multispecific antibody.

FIG. 41A shows a schematic illustration of arrangement of antibody variable domains in different CODVs. The domains are dotted, striped, grid, and gray for Fv182.1 (182.1), Fv46.1 (46.1), Fv61.1 (61.1) and constant domains, respectively.

FIG. 41B shows combinations of RBD mutations on the SARS-CoV-2 spike designed to distinguish different binding modes of CODV.

FIG. 41C is a schematic of CODV 61.1-182.1 (left) and binding of the CODV 61.1-182.1 to spike protein mutants (middle left and right). For clarity, the Fv domain that is not able to bind to the mutant is colored black. CODV binding to K444E/L452R mutant was observed, indicating CODV 61.1-182.1 can bind to spike protein through Fv182 domain (middle left). However, no CODV was observed to bind the L452R/F486S mutant (middle right), suggesting that the CODV 61.1-182.1 cannot bind through the Fv61 domain in the trimer context. A model of the CODV 61.1-182.1 bound to the SARS-CoV-2 spike protein is shown in the allowed Fv182-binding mode (right). For clarity, only one CODV is shown to bind to the spike protein in RBD-up conformation. The white scale bar represents 10 nm.

FIG. 41D is a schematic of CODV 182.1-61.1 (left) and binding of the CODV 182.1-61.1 to spike protein mutants (middle left and right). For clarity, the Fv domain that is not able to bind to the mutant is colored black. CODV was observed to bind both K444E/L452R and L452R/F486S mutants, indicating the CODV 182.1-61.1 can bind to spike protein through either Fv182 domain (middle left) or Fv61 domain (middle right). Models of the CODV 182.1-61.1 bound to the SARS-CoV-2 spike protein are shown in both Fv182.1-binding and Fv61.1-binding modes (right). The scale is the same as in FIG. 41C.

FIG. 41E is a schematic of CODV 46.1-182.1 (left) and binding of the CODV 46.1-182.1 to spike protein mutants (middle left and right). For clarity, the Fv domain that is not able to bind to the mutant is colored black. CODV was observed to bind both K444E/L452R and K444E/F486S mutants, indicating the CODV 46.1-182.1 can bind to the spike protein through both Fv182 and Fv46 domains (middle left and right). Models of the CODV 46.1-182.1 bound to the SARS-CoV-2 spike protein are shown to bind through Fv182 and Fv46-binding modes (right). The scale is the same as in FIG. 41C.

FIG. 41F is a schematic of CODV 182.1-46.1 (left) and binding of the CODV 182.1-46.1 to spike protein mutants (middle left and right). For clarity, the Fv domain that is not able to bind to the mutant is colored black. CODV was observed to bind K444E/L452R, indicating the CODV 182.1-46.1 can bind to the spike protein through the Fv182.1 domain (middle left). However, no CODV was observed to bind the K444E/F486S mutant (middle right), suggesting that the CODV 182.1-46.1 cannot bind through the Fv46 domain in the trimer context. Model of the CODV 182.1-46.1 bound to the SARS-CoV-2 spike protein is shown to bind through the allowed Fv182 domain (right). The scale is the same as in FIG. 41C.

FIG. 41G shows CODV 46.1-182.1- and 182.1-46.1-induced aggregation of the Omicron spike protein using negative stain-electron microscopy (NSEM).

FIG. 41H shows the spike-aggregating potential of 61.1/46.1-182.1 using NSEM.

FIG. 42 shows results from three independent experiments showing SARS-CoV-2 escape potential of T3_V3 (having 61.1, 46.1 and 182.1 binding regions), compared to an antibody having binding regions for 182.1, 61.1 and 46.1 (182.1+61.1+46.1); an antibody having binding regions for 182.1 (182.1); bebtelovimab (LY1404); and an AZD7442 cocktail (2196+2130).

FIG. 43 shows the resulting serum concentration of E8 glycosylation mutant antibodies in g/mL, at days 1, 2, 5, 7, 9, 14, 21, 28 and 35, following infusion of mice at 5 mg antibody/kg of body weight.

### DETAILED DESCRIPTION

Provided herein are antigen binding polypeptides and polypeptide complexes having improved features. In some aspects, the antigen binding polypeptides and polypeptide complexes have broad reactivity against SARS-CoV-2 variants of concern (VOCs) and/or resistance to SARS-CoV-2 escape. In some aspects, the antigen binding polypeptides and polypeptide complexes provided herein are easier to manufacture and administer than current therapeutic antibody cocktails. In some aspects, the antigen binding polypeptides and polypeptide complexes treat and/or prevent SARS-CoV-2 infection and/or COVID-19 in immunocompromised individuals who cannot mount an effective immune response through vaccinations.

Various terms relating to aspects of disclosure are used throughout the specification and claims. Such terms are to be given their ordinary meaning in the art, unless otherwise indicated. Other specifically defined terms are to be construed in a manner consistent with the definitions provided herein.

### Definitions

As used herein, the term “SARS-CoV-2” refers to “severe acute respiratory syndrome coronavirus 2.” SARS-CoV-2 is the strain of coronavirus that causes coronavirus disease 2019 (COVID-19).

As used herein, the term “SARS-CoV-2 virus” includes to the SARS-CoV-2 virus, virion or fragment thereof.

As used herein, the term “COVID-19” refers to “coronavirus disease 2019.” COVID-19 is caused by the SARS-CoV-2 virus. Symptoms of COVID-19 are variable, but often include fever, cough, headache, fatigue, breathing difficulties, loss of smell, and loss of taste. Symptoms may begin one to fourteen days after exposure to the virus; however, at least a third of people who are infected do not develop noticeable symptoms. Of those people who develop symptoms noticeable enough to be classed as patients, most develop mild to moderate symptoms (up to mild pneumonia), with approximately 15% developing severe symptoms (dyspnea, hypoxia, or more than 50% lung involvement on imaging), and approximately 5% suffering critical symptoms (respiratory failure, shock, or multiorgan dysfunction). Some people continue to experience a range of effects for months after recovery (“long COVID”).

As used herein, the term “SARS-CoV-2 protein” refers to any protein related to the SARS-CoV-2 virus. The SARS-CoV-2 virus is made of 16 non-structural proteins and four structural proteins. The non-structural proteins are involved mainly in virus replication. The structural proteins include the membrane protein, nucleocapsid protein, envelope protein, and spike protein, which are involved in assembly and infection of the virus. This has two subunits, S1 and S2, which are responsible for the specific recognition of human angiotensin converting enzyme 2 (hACE2) on the surface of host cells and viral entry. S1 specifically binds the hACE2 through its receptor-binding domain (RBD), whereas S2 facilitates the fusion of the virus to the cell membrane. As such, as used herein, a SARS-CoV-2 protein includes, but is not limited to, a membrane protein, nucleocapsid protein, envelope protein, or spike protein, or a combination thereof. In some aspects, the SARS-CoV-2 protein is a spike protein.

In some aspects, an antigen binding polypeptide or antigen binding polypeptide complex provided herein (e.g., an antibody or antigen binding fragment thereof) is, or is part of, a class I antibody, a class II antibody, or a class III antibody.

As used herein, “class I” refers to an antibody that typically contains a short heavy chain CDR3 region and competes with angiotensin-converting enzyme 2 (ACE2) for binding to the receptor binding domain (RBD) of the spike protein, and only recognizes the “up” configuration of the RBD. See, e.g., Shrestha et al., Front. Immunol. 12:7520003, 2021. As used herein, “class II” refers to an antibody that typically has a long heavy chain CDR3 loop, competes with ACE2 for binding to RBD, and is capable of binding to RBD in both the “up” and “down” configurations. As used herein, “class III” refers to an antibody that is capable of binding to RBD in both the “up” and “down” configurations and typically binds outside of the ACE2 binding region. As used herein, “class IV” refers to an antibody that binds to the highly conserved, cryptic epitope on RBD outside the receptor binding motif (RBM) of the spike protein.

In some aspects, an antigen binding polypeptide or antigen binding polypeptide complex provided herein (e.g., an antibody or antigen binding fragment thereof) belongs to two, three or four classes. Accordingly, in some aspects, an antigen binding polypeptide or antigen binding polypeptide complex provided herein (e.g., an antibody or antigen binding fragment thereof) is a class I and II antibody, class I and III antibody, class I and IV antibody, class II and III antibody, class II and IV antibody, class III and IV antibody, class I, II and III antibody, class I, II and IV antibody, class I, III and IV antibody, class II, III and IV antibody, or class I, II, III and IV antibody.

In some aspects, an antigen binding polypeptide or antigen binding polypeptide complex provided herein comprises one or more class I antigen binding sites, one or more class II antigen binding sites, and/or one or more class III antigen binding sites. In some aspects, VH1 and VL1 and/or VH3 and VL3 are class III antigen binding sites. In some aspects, VH2 and VL2 and/or VH4 and VL4 are class I antigen binding sites. In some aspects, VH1, VL1, VH3 and VL3 are class III antigen binding sites and VH2, VL2, VH4 and VL4 are class I antigen binding sites. In some aspects, VH1 and VL1 are class II antigen binding sites. In some aspects, VH2 and VL2 are class I antigen binding sites. In some aspects, VH3 and VL3 are class I antigen binding sites. In some aspects, VH4 and VL4 are class III antigen binding sites. In some aspects, VH1 and VL1 are class II antigen binding sites, VH2 and VL2 are class I antigen binding sites, VH3 and VL3 are class I antigen binding sites, and VH4 and VL4 are class III antigen binding sites.

As used herein, the term “spike protein” refers to the largest of the four structural proteins found in the SARS-CoV-2 virus. The spike protein assembles into trimers that form large structures, called spikes or peplomers, that project from the surface of the SARS-CoV-2 virion and mediate entry of the virion into host cells. The SARS-CoV-2 spike protein is also called “spike (S) glycoprotein” and E2. The structural and functional properties of this protein have been characterized. See, e.g., Huang et al., Acta Pharmacol. Sin. 41:1141-1149, 2020; Wu et al., Nature 579(7798):265-269, 2020; and Gene ID: 43740568.

Antigen binding sequences (e.g., CDR, VH, VL, heavy chain and light chain sequences from antibodies) for anti-SARS-CoV-2 proteins are known. Such sequences include, but are not limited to SEQ ID NOs:38-788.

As used herein, the term “antigen binding polypeptide” refers to a polypeptide having the ability to specifically bind to one or more substances that induce an immune response (i.e., one or more antigens or epitopes).

As used herein, the term “antigen binding polypeptide complex” refers to a group of two, three, four, or more associated polypeptides, wherein at least one polypeptide has the ability to specifically bind to one or more antigens. An antigen binding polypeptide complex, includes, but is not limited to, an antibody or antigen binding fragment thereof.

The term “antibody” includes, without limitation, a glycoprotein immunoglobulin which binds specifically to an antigen and comprises at least two heavy (H) chains and two light (L) chains interconnected by disulfide bonds. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. A heavy chain may have the C-terminal lysine or not. Unless specified otherwise herein, the amino acids in the variable regions are numbered using the Kabat numbering system and those in the constant regions are numbered using the EU system.

The term “monoclonal antibody,” as used herein, refers to an antibody that is produced by a single clone of B-cells and binds to the same epitope. In contrast, the term “polyclonal antibody” refers to a population of antibodies that are produced by different B-cells and bind to different epitopes of the same antigen. The term “antibody” includes, by way of example, monoclonal and polyclonal antibodies; chimeric and humanized antibodies; human or non-human antibodies; wholly synthetic antibodies; and single chain antibodies. A non-human antibody can be humanized by recombinant methods to reduce its immunogenicity in man.

The antibody can be an antibody that has been altered (e.g., by mutation, deletion, substitution, conjugation to a non-antibody moiety). For example, an antibody can include one or more variant amino acids (compared to a naturally occurring antibody) which change a property (e.g., a functional property) of the antibody. For example, several such alterations are known in the art which affect, e.g., half-life, effector function, and/or immune responses to the antibody in a patient. The term antibody also includes artificial polypeptide constructs which comprise at least one antibody-derived antigen binding site.

An “antigen binding fragment” of an antibody refers to one or more fragments or portions of an antibody that retain the ability to bind specifically to the antigen bound by the whole antibody. It has been shown that the antigen binding function of an antibody can be performed by fragments or portions of a full-length antibody. An antigen binding fragment can contain the antigenic determining regions of an intact antibody (e.g., the complementarity determining regions (CDRs)). Examples of antigen binding fragments of antibodies include, but are not limited to, Fab, Fab′, F(ab′)₂, and Fv fragments, linear antibodies, and single chain antibodies. An antigen binding fragment of an antibody can be derived from any animal species, such as rodents (e.g., mouse, rat, or hamster) and humans or can be artificially produced.

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody.

Antigen binding fragments are obtained using conventional techniques known to those with skill in the art, and the fragments screened for utility in the same manner as are intact antibodies. Antigen binding fragments can be produced by recombinant DNA techniques, or by enzymatic or chemical cleavage of intact immunoglobulins.

As used herein, the term “variable region” typically refers to a portion of an antibody, generally, a portion of a light or heavy chain, typically about the amino-terminal 110 to 120 amino acids, or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of an antibody with antigen. In some aspects, the variable region is a mammalian variable region, e.g., a human, mouse or rabbit variable region. In some aspects, the variable region comprises rodent or murine CDRs and human framework regions (FRs). In some aspects, the variable region is a primate (e.g., non-human primate) variable region. In some aspects, the variable region comprises rodent or murine CDRs and primate (e.g., non-human primate) framework regions (FRs).

The terms “complementarity determining region” or “CDR”, as used herein, refer to each of the regions of an antibody variable domain which are hypervariable in sequence and/or form structurally defined loops (hypervariable loops) and/or contain the antigen-contacting residues. Antibodies can comprise six CDRs, e.g., three in the VH and three in the VL.

The terms “VL”, “VL region,” and “VL domain” are used herein interchangeably to refer to the light chain variable region of an antigen binding polypeptide, antigen binding polypeptide complex, antibody or antigen binding fragment thereof. In some aspects, a VL region is referred to herein as VL1 to denote a first light chain variable region, VL2 to denote a second light chain variable region, VL3 to denote a third light chain variable region, and VL4 to denote a fourth light chain variable region. An enumerated VL region (e.g., VL1) can have the same or different antigen binding properties and/or the same or different sequence as another enumerated VL region (e.g., VL2).

The terms “VH”, “VH region,” and “VH domain” are used herein interchangeably to refer to the heavy chain variable region of an antigen binding polypeptide, antigen binding polypeptide complex, antibody or antigen binding fragment thereof. In some aspects, a VH region is referred to herein as VH1 to denote a first heavy chain variable region, VH2 to denote a second heavy chain variable region, VH3 to denote a third heavy chain variable region, and VH4 to denote a fourth heavy chain variable region. An enumerated VH region (e.g., VH1) can have the same or different antigen binding properties and/or the same or different sequence as another enumerated VH region (e.g., VH2).

As used herein, “Kabat numbering” and like terms are recognized in the art and refer to a system of numbering amino acid residues in the heavy and light chain variable regions of an antibody or antigen binding fragment thereof. In some aspects, CDRs can be determined according to the Kabat numbering system (see, e.g., Kabat E A & Wu T T (1971) Ann NY Acad Sci 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3).

As used herein, the terms “constant region” or “constant domain” are used interchangeably to refer to a portion of an antigen binding polypeptide, antigen binding polypeptide complex, antibody or antigen binding fragment thereof, e.g., a carboxyl terminal portion of a light and/or heavy chain which is not directly involved in binding of an antibody to antigen but which can exhibit various effector functions, such as interaction with the Fc region. The constant region generally has a more conserved amino acid sequence relative to a variable region. In some aspects, an antigen binding polypeptide, antigen binding polypeptide complex, antibody or antigen binding fragment thereof comprises a constant region or portion thereof that is sufficient for antibody-dependent cell-mediated cytotoxicity (ADCC), antibody-dependent cellular phagocytosis (ADCP), and complement-dependent cytotoxicity (CDC). A constant region includes, but is not limited to, a light chain constant region (CL) or heavy chain constant region (CH1, CH2, CH3).

As used herein, the terms “fragment crystallizable region,” “Fc region,” or “Fc domain” are used interchangeably herein to refer to the tail region of an antibody that interacts with cell surface receptors called Fc receptors and some proteins of the complement system. Fc regions typically comprise CH2 and CH3 regions, and, optionally, an immunoglobulin hinge.

As used herein, the terms “immunoglobulin hinge,” “hinge,” “hinge domain” or “hinge region” are used interchangeably to refer to a stretch of heavy chains between the Fab and Fc portions of an antigen binding polypeptide, antigen binding polypeptide complex, antibody or antigen binding fragment thereof. A hinge provides structure, position and flexibility, which assist with normal functioning of antibodies (e.g., for crosslinking two antigens or binding two antigenic determinants on the same antigen molecule). An immunoglobulin hinge is divided into upper, middle and lower hinge regions that can be separated based on structural and/or genetic components. An immunoglobulin hinge of the invention can contain one, two or all three of these regions. Structurally, the upper hinge region stretches from the C terminal end of CH1 to the first hinge disulfide bond. The middle hinge region stretches from the first cysteine to the last cysteine in the hinge. The lower hinge region extends from the last cysteine to the glycine of CH2. The cysteines present in the hinge form interchain disulfide bonds that link the immunoglobulin monomers.

As used herein, the term “Fab” refers to a region of an antibody that binds to an antigen. It is typically composed of one constant and one variable domain of each of the heavy and the light chain.

As used herein, the term “heavy chain” refers to a portion of an antigen binding polypeptide, antigen binding polypeptide complex, antibody or antigen binding fragment thereof typically composed of a heavy chain variable region (VH), a heavy chain constant region 1 (CH1), a heavy chain constant region 2 (CH2), and a heavy chain constant region 3 (CH3). A typical antibody is composed of two heavy chains and two light chains. When used in reference to an antibody, a heavy chain can refer to any distinct type, e.g., alpha (a), delta (S), epsilon (F), gamma (γ), and mu (p), based on the amino acid sequence of the constant region, which gives rise to IgA, IgD, IgE, IgG, and IgM classes of antibodies, respectively, including subclasses of IgG, e.g., IgG1, IgG2, IgG3, and IgG4. Heavy chain amino acid sequences are known in the art. In some aspects, the heavy chain is a human heavy chain.

As used herein, the term “light chain” refers to a portion of an antigen binding polypeptide, antigen binding polypeptide complex, antibody or antigen binding fragment thereof typically composed of a light chain variable region (VL) and a light chain constant region (CL). A typical antibody is composed of two light chains and two heavy chains. When used in reference to an antibody, a light chain can refer to any distinct type, e.g., kappa (x) or lambda (k), based on the amino acid sequence of the constant region. Light chain amino acid sequences are known in the art. In some aspects, the light chain is a human light chain.

The term “chimeric” antibody or antigen binding fragment thereof refers to an antibody or antigen binding fragments thereof wherein the amino acid sequence is derived from two or more species. Typically, the variable region of both light and heavy chains corresponds to the variable region of antibodies or antigen binding fragments thereof derived from one species of mammals (e.g., mouse, rat, rabbit, etc.) with the desired specificity, affinity and capability, while the constant regions are homologous to the sequences in antibodies or antigen binding fragments thereof derived from another (usually human) to avoid eliciting an immune response in that species.

The term “humanized” antibody or antigen binding fragment thereof refers to forms of non-human (e.g., murine) antibodies or antigen binding fragments that are specific immunoglobulin chains, chimeric immunoglobulins, or fragments thereof that contain minimal non-human (e.g., murine) sequences. Typically, humanized antibodies or antigen binding fragments thereof are human immunoglobulins in which residues from a complementary determining region (CDR) are replaced by residues from a CDR of a non-human species (e.g., mouse, rat, rabbit, hamster) that have the desired specificity, affinity, and capability (Jones et al., Nature 321:522-525 (1986); Riechmann et al., Nature 332:323-327 (1988); Verhoeyen et al., Science 239:1534-1536 (1988)). In some aspects, the Fv framework region (FR) residues of a human immunoglobulin are replaced with the corresponding residues in an antibody or fragment from a non-human species that has the desired specificity, affinity, and capability. The humanized antibody or antigen binding fragment thereof can be further modified by the substitution of additional residues either in the Fv framework region and/or within the replaced non-human residues to refine and optimize antibody or antigen-binding fragment thereof specificity, affinity, and/or capability. In general, a humanized antibody or antigen binding fragment thereof will comprise substantially all of at least one, and typically two or three, variable domains containing all or substantially all of the CDR regions that correspond to the non-human immunoglobulin whereas all or substantially all of the FR regions are those of a human immunoglobulin consensus sequence. A humanized antibody or antigen binding fragment thereof can also comprise at least a portion of a constant region, typically that of a human immunoglobulin. Examples of methods used to generate humanized antibodies are known and described, for example, in U.S. Pat. No. 5,225,539; Roguska et al., Proc. Natl. Acad. Sci., USA, 91(3):969-973 (1994), and Roguska et al., Protein Eng. 9(10):895-904 (1996).

The term “human” antibody or antigen binding fragment thereof, as used herein, means an antibody or antigen binding fragment thereof having an amino acid sequence derived from a human immunoglobulin gene locus, where such antibody or antigen binding fragment is made using recombinant techniques known in the art. This definition of a human antibody or antigen binding fragment thereof includes intact or full-length antibodies and fragments thereof.

A polypeptide, polypeptide complex, antibody, antigen binding fragment thereof, polynucleotide, vector or host cell which is “isolated” is a polypeptide, polypeptide complex, antibody, antigen binding fragment thereof, polynucleotide, vector or host cell which is in a form not found in nature. Isolated polypeptides, polypeptide complexes, antibodies, antigen binding fragments thereof, polynucleotides, vectors or host cells include those which have been purified to a degree that they are no longer in a form in which they are found in nature. In some aspects, a polypeptide, polypeptide complex, antibody, antigen binding fragment thereof, polynucleotide, vector or host cell which is isolated is substantially pure. As used herein, “substantially pure” refers to material which is at least 50% pure (i.e., free from contaminants), at least 90% pure, at least 95% pure, at least 98% pure, or at least 99% pure.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in some aspects, the polypeptides can occur as single chains or associated chains.

As used herein, an “isoelectric point” or “pI” refer to the pH at which the net charge of a polypeptide (e.g., an antigen binding polypeptide or antigen binding polypeptide complex provided herein) is zero.

As used herein, “pH” refers to a quantitative measure of acidity or basicity. This term translates the values of the concentration of the hydrogen ion-which ordinarily ranges between about 1 and 10⁻¹⁴ gram-equivalents per litre-into numbers between 0 and 14.

As used herein, “SARS-CoV-2 escape” refers to the process by which a SARS-CoV-2 virus acquires one or more mutations that allow the virus to evade the antibodies generated by a particular vaccine.

As used herein, “SARS-CoV-2 evolution” refers to the heritable genetic changes that a SARS-CoV-2 virus accumulates during its life time, which can arise from adaptations in response to environmental changes or the immune response of the host.

The use of the alternative (e.g., “or”) should be understood to mean either one, both, or a combination thereof of the alternatives. As used herein, the indefinite articles “a” or “an” should be understood to refer to “one or more” of any recited or enumerated component.

As used herein, the term “and/or” is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” “having,” or the like, otherwise analogous aspects described in terms of “consisting of” and/or “consisting essentially of” are also provided.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” can mean within 1 or more than 1 standard deviation per the practice in the art. Alternatively, “about” can mean a range of up to 10% or 20% (i.e., ±10% or ±20%). For example, about 3 mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” should be assumed to be within an acceptable error range for that particular value or composition.

As described herein, any numerical range, concentration range, percentage range, ratio range or integer range is to be understood to include the value of any integer within the recited range and, when appropriate, fractions thereof (such as one-tenth and one-hundredth of an integer), unless otherwise indicated.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 5th ed., 2013, Academic Press; and the Oxford Dictionary Of Biochemistry And Molecular Biology, 2006, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined herein are more fully defined by reference to the specification in its entirety.

Various aspects are described in further detail in the following sections.

### Antigen Binding Polypeptides and Polypeptide Complexes

Provided herein are antigen binding polypeptides and polypeptide complexes having certain structural and/or functional features.

For example, provided herein is a multispecific antibody selected from the group consisting of:
(a) an antigen binding polypeptide having a structure represented by:
   VL1-L1-VH1 or VH1-L2-VL1;
wherein:
   VL1 is a first immunoglobulin light chain variable region that specifically binds to a severe acute respiratory syndrome coronavirus 2 (SARS-CoV-2) protein;
   VH1 is a first immunoglobulin heavy chain variable region that specifically binds to a SARS-CoV-2 protein; and
   L1 and L2 are amino acid linkers; or
an antigen binding polypeptide having a structure represented by:
   VL1-L1-VL2-L2-VH2-L3-VH1;
   VL1-L1-VH2-L2-VL2-L3-VH1;
   VH1-L4-VH2-L5-VL2-L6-VL1; or
   VH1-L4-VL2-L5-VH2-L6-VL1;
wherein:
   VL1 is a first immunoglobulin light chain variable region that specifically binds to a SARS-CoV-2 protein;
   VL2 is a second immunoglobulin light chain variable region that specifically binds to a SARS-CoV-2 protein;
   VH1 is a first immunoglobulin heavy chain variable region that specifically binds to a SARS-CoV-2 protein;
   VH2 is a second immunoglobulin heavy chain variable region that specifically binds to a SARS-CoV-2 protein; and
   L1-L6 are amino acid linkers; and
wherein (a) is selected from any one of the more specific embodiments provided herein for an antigen binding polypeptide in an antigen binding polypeptide complex having no more than three polypeptide chains;
(b) an antigen binding polypeptide complex comprising a first polypeptide, a second polypeptide, a third polypeptide, and a fourth polypeptide;
wherein the first polypeptide has a structure represented by:
   VL1-L1-CL;
wherein the second polypeptide has a structure represented by:
   VH1-L2-CH1;
wherein the third polypeptide has a structure represented by:
   VH2-L3-VH3-L4-CH1;
wherein the fourth polypeptide has a structure represented by:
   VL3-L5-VL2-L6-CL; and
wherein:
   VL1 is a first immunoglobulin light chain variable region that specifically binds to a SARS-CoV-2 protein;
   VL2 is a second immunoglobulin light chain variable region that specifically binds to a SARS-CoV-2 protein;
   VL3 is a third immunoglobulin light chain variable region that specifically binds to a SARS-CoV-2 protein;
   VH1 is a first immunoglobulin heavy chain variable region that specifically binds to a SARS-CoV-2 protein;
   VH2 is a second immunoglobulin heavy chain variable region that specifically binds to a SARS-CoV-2 protein;
   VH3 is a third immunoglobulin heavy chain variable region that specifically binds to a SARS-CoV-2 protein;
   CH1 is an immunoglobulin heavy chain constant region 1;
   CL is an immunoglobulin light chain constant region; and
   L1-L6 are amino acid linkers; and
(c) an antigen binding polypeptide having a structure represented by:
   VL1-L1-VH1 or VH1-L2-VL1;
wherein:
   VL1 is a first immunoglobulin light chain variable region that specifically binds to a SARS-CoV-2 protein;
   VH1 is a first immunoglobulin heavy chain variable region that specifically binds to a SARS-CoV-2 protein; and
   L1 and L2 are amino acid linkers; or
an antigen binding polypeptide having a structure represented by:
   VL1-L1-VL2-L2-VH2-L3-VH1;
   VL1-L1-VH2-L2-VL2-L3-VH1;
   VH1-L4-VH2-L5-VL2-L6-VL1; or
   VH1-L4-VL2-L5-VH2-L6-VL1;
wherein:
   VL1 is a first immunoglobulin light chain variable region that specifically binds to a SARS-CoV-2 protein;
   VL2 is a second immunoglobulin light chain variable region that specifically binds to a SARS-CoV-2 protein;
   VH1 is a first immunoglobulin heavy chain variable region that specifically binds to a SARS-CoV-2 protein;
   VH2 is a second immunoglobulin heavy chain variable region that specifically binds to a SARS-CoV-2 protein; and
   L1-L6 are amino acid linkers;
wherein (c) is selected from any one of the more specific embodiments provided herein for an antigen binding polypeptide in an antigen binding polypeptide complex having no more than three polypeptide chains; and
wherein the antigen binding polypeptide further comprises one or more immunoglobulin heavy chain constant region 1 (CH1) and/or one or more immunoglobulin light chain constant region (CL) between any one of the variable regions and/or amino acid linkers (e.g., between VL1 and L1, between L1 and VH1, between VH1 and L2, between L2 and VL1, between L1 and VL2, between VL2 and L2, between L2 and VH2, between VH2 and L3, between L3 and VH1, between VL1 and L1, between L1 and VH2, between VH2 and L2, between, L2 and VL2, between VL2 and L3, between L3 and VH1, between VH1 and L4, between L4 and VH2, between VH2 and L5, between L5 and VL2, between VL2 and L6, between L6 and VL1, between VH1 and L4, between L4 and VL2, between L4 and VL2, between VL2 and L5, between L5 and VH2, between VH2 and L6, or between L6 and VL1).

Also provided herein is a multispecific antibody comprising a first polypeptide, a second polypeptide, a third polypeptide, and a fourth polypeptide;
wherein the first polypeptide has a structure represented by VL1-L1-CL;
wherein the second polypeptide has a structure represented by VH1-L2-CH1-L3-Fc;
wherein the third polypeptide has a structure represented by VH2-L4-VH3-L5-CH1-L6-Fc;
wherein the fourth polypeptide has a structure represented by VL3-L7-VL2-L8-CL; and
wherein VL1 is a first immunoglobulin light chain variable region that specifically binds to a SARS-CoV-2 protein;
VL2 is a second immunoglobulin light chain variable region that specifically binds to a SARS-CoV-2 protein;
VL3 is a third immunoglobulin light chain variable region that specifically binds to a SARS-CoV-2 protein;
VH1 is a first immunoglobulin heavy chain variable region that specifically binds to a SARS-CoV-2 protein;
VH2 is a second immunoglobulin heavy chain variable region that specifically binds to a SARS-CoV-2 protein;
VH3 is a third immunoglobulin heavy chain variable region that specifically binds to a SARS-CoV-2 protein;
Fc is a region comprising an immunoglobulin heavy chain constant region 2 (CH2), an immunoglobulin heavy chain constant region 3 (CH3), and optionally, an immunoglobulin hinge;
CH1 is an immunoglobulin heavy chain constant region 1;
CL is an immunoglobulin light chain constant region; and
L1-L8 are amino acid linkers.

In some aspects, the VH1, VH2 and/or VH3 (VH1; VH2; VH3; VH1 and VH2; VH1 and VH3; VH2 and VH3; VH1, VH2 and VH3) of the multispecific antibody comprises: a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666; and/or the VL1, VL2 and/or VL3 (VL1; VL2; VL3; VL1 and VL2; VL1 and VL3; VL2 and VL3; VL1, VL2 and VL3) of the multispecific antibody comprises: a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666. In some preferred aspects, CDR1, CDR2 and CDR3 of each of VH1, VH2, VH3, VL1, VL2 and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VL1, VL2 and VL3 comprise or consist of a corresponding amino acid sequence specified above.

In some aspects, VH1 and VH3 each comprise the same CDR1, CDR2 and/or CDR3 sequences (e.g., a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666, or any other CDR sequence within a larger VH or full length polypeptide chain sequence disclosed herein), and/or VL1 and VL3 each comprise the same CDR1, CDR2 and/or CDR3 sequences (e.g., a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666, or any other CDR sequence within a larger VH or full length polypeptide chain sequence disclosed herein). In some preferred aspects, CDR1, CDR2 and CDR3 of each of VH1, VH3, VL1 and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH3, VL1 and VL3 comprise or consist of a corresponding amino acid sequence specified above.

In some aspects, VH1 and VH2 each comprise the same CDR1, CDR2 and/or CDR3 sequences (e.g., a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666, or any other CDR sequence within a larger VH or full length polypeptide chain sequence disclosed herein), and/or VL1 and VL2 each comprise the same CDR1, CDR2 and/or CDR3 sequences (e.g., a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666, or any other CDR sequence within a larger VH or full length polypeptide chain sequence disclosed herein). In some preferred aspects, CDR1, CDR2 and CDR3 of each of VH1, VH2, VL1 and VL2 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VL1 and VL2 comprise or consist of a corresponding amino acid sequence specified above.

In some aspects, VH2 and VH3 each comprise the same CDR1, CDR2 and/or CDR3 sequences (e.g., a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666, or any other CDR sequence within a larger VH or full length polypeptide chain sequence disclosed herein), and/or VL2 and VL3 each comprise the same CDR1, CDR2 and/or CDR3 sequences (e.g., a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666, or any other CDR sequence within a larger VH or full length polypeptide chain sequence disclosed herein). In some preferred aspects, CDR1, CDR2 and CDR3 of each of VH2, VH3, VL2 and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH2, VH3, VL2 and VL3 comprise or consist of a corresponding amino acid sequence specified above.

In some aspects, VH1, VH2 and VH3 each comprise the same CDR1, CDR2 and/or CDR3 sequences (e.g., a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666, or any other CDR sequence within a larger VH or full length polypeptide chain sequence disclosed herein), and VL1, VL2 and VL3 each comprise the same CDR1, CDR2 and/or CDR3 sequences (e.g., a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666, or any other CDR sequence within a larger VH or full length polypeptide chain sequence disclosed herein). In some preferred aspects, CDR1, CDR2 and CDR3 of each of VH1, VH2, VH3, VL1, VL2 and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VL1, VL2 and VL3 comprise or consist of a corresponding amino acid sequence specified above

In some aspects, VH1, VH2 and VH3 each comprise different CDR1, CDR2 and/or CDR3 sequences (e.g., a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666, or any other CDR sequence within a larger VH or full length polypeptide chain sequence disclosed herein), and VL1, VL2 and VL3 each comprise different CDR1, CDR2 and/or CDR3 sequences (e.g., a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666, or any other CDR sequence within a larger VH or full length polypeptide chain sequence disclosed herein). In some preferred aspects, CDR1, CDR2 and CDR3 of each of VH1, VH2, VH3, VL1, VL2 and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VL1, VL2 and VL3 comprise or consist of a corresponding amino acid sequence specified above.

In some aspects, VH1 comprises the same heavy chain variable region as VH3. In some aspects, VL1 comprises the same light chain variable region as VL3. In some aspects, VH1 comprises the same heavy chain variable region as VH3, and VL1 comprises the same light chain variable region as VL3.

In some aspects, VH1 comprises the same heavy chain variable region as VH2. In some aspects, VL1 comprises the same light chain variable region as VL2. In some aspects, VH1 comprises the same heavy chain variable region as VH2, and VL1 comprises the same light chain variable region as VL2.

In some aspects, VH2 comprises the same heavy chain variable region as VH3. In some aspects, VL2 comprises the same light chain variable region as VL3. In some aspects, VH2 comprises the same heavy chain variable region as VH3, and VL2 comprises the same light chain variable region as VL3.

In some aspects, VH1, VH2 and VH3 each comprise the same heavy chain variable region. In some aspects, VL1, VL2 and VL3 each comprise the same light chain variable region. In some aspects, VH1, VH2 and VH3 each comprise the same heavy chain variable region, and VL1, VL2 and VL3 each comprise the same light chain variable region.

In some aspects, VH1, VH2 and VH3 each comprise different heavy chain variable regions. In some aspects, VL1, VL2 and VL3 each comprise different light chain variable regions. In some aspects, VH1, VH2 and VH3 each comprise different heavy chain variable regions, and VL1, VL2 and VL3 each comprise different light chain variable regions.

In some aspects, VH1, VH2 and/or VH3 comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:42, 50, 58, 66, 74, 170, 178, 186, 263, 271, 279, 287, 510, 512, 520, 528, 536, 544, 552, 560, 568, 576, 584, 592, 600, 608, 616, 624, 626, 628, 630, 632, 634, 636, 638, 646, 654, 663, 779, 781, 785 and 787; and/or VL1, VL2 and/or VL3 comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:38, 46, 54, 62, 70, 166, 174, 182, 259, 267, 275, 283, 508, 509, 511, 516, 524, 532, 540, 548, 556, 564, 572, 580, 588, 596, 604, 612, 620, 625, 627, 629, 631, 633, 635, 637, 642, 650, 659 and 667. In some preferred aspects, VH1, VH2, VH3, VL1, VL2 and VL3 have at least 90% identity with the corresponding amino acid sequences specified above. In some particularly preferred aspects, VH1, VH2, VH3, VL1, VL2 and VL3 comprise or consist of the corresponding amino acid sequences specified above. As used herein, “at least 90% identity” includes at least 91%, 92%, 93%, 94%, 95%, 96%, 97%, 98%, 99% and 100% identity to the recited reference sequence.

In some aspects, an antigen binding polypeptide provided herein has a structure represented by VL1-L1-VH1 or VH1-L2-VL1; wherein VL1 is a first immunoglobulin light chain variable region that specifically binds to a severe acute respiratory syndrome coronavirus 2 (SARS-CoV-2) protein; VH1 is a first immunoglobulin heavy chain variable region that specifically binds to a SARS-CoV-2 protein; and L1 and L2 are amino acid linkers.

In some aspects, an antigen binding polypeptide provided herein has a structure represented by VL1-L1-VL2-L2-VH2-L3-VH1; VL1-L1-VH2-L2-VL2-L3-VH1; VH1-L4-VH2-L5-VL2-L6-VL1; or VH1-L4-VL2-L5-VH2-L6-VL1; wherein VL1 is a first immunoglobulin light chain variable region that specifically binds to a SARS-CoV-2 protein; VL2 is a second immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; and L1-L6 are amino acid linkers. In some aspects, the antigen binding polypeptide further comprises an Fc region, CH1 region, CL region, or a combination thereof. In some aspects, the antigen binding polypeptide is part of an antigen binding polypeptide complex provided herein (e.g., antibody or antigen binding fragment thereof).

In some aspects, an antigen binding polypeptide provided herein has a structure represented by VL1-L1-VL2-L2-VH2-L3-VH1 or VH1-L4-VH2-L5-VL2-L6-VL1; wherein VL1 is a first immunoglobulin light chain variable region that specifically binds to a severe acute respiratory syndrome coronavirus 2 (SARS-CoV-2) protein; VL2 is a second immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; and L1-L6 are amino acid linkers. In some aspects, the antigen binding polypeptide further comprises an Fc region, CH1 region, CL region, or a combination thereof. In some aspects, the antigen binding polypeptide is part of an antigen binding polypeptide complex provided herein (e.g., antibody or antigen binding fragment thereof).

In some aspects, an antigen binding polypeptide provided herein has a structure represented by VL1-L1-VH2-L2-VL2-L3-VH1 or VH1-L4-VL2-L5-VH2-L6-VL1; wherein VL1 is a first immunoglobulin light chain variable region that specifically binds to a severe acute respiratory syndrome coronavirus 2 (SARS-CoV-2) protein; VL2 is a second immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; and L1-L6 are amino acid linkers. In some aspects, the antigen binding polypeptide further comprises an Fc region, CH1 region, CL region, or a combination thereof. In some aspects, the antigen binding polypeptide is part of an antigen binding polypeptide complex provided herein (e.g., antibody or antigen binding fragment thereof).

In some aspects, an antigen binding polypeptide complex provided herein comprises a first polypeptide and a second polypeptide; wherein the first polypeptide has a structure represented by VL1-L1-VH1 or VH1-L2-VL1; wherein the second polypeptide has a structure represented by VL2-L3-VH2 or VH2-L4-VL2; wherein VL1 is a first immunoglobulin light chain variable region that specifically binds to a SARS-CoV-2 protein; VH1 is a first immunoglobulin heavy chain variable region that specifically binds to a SARS-CoV-2 protein; wherein VL2 is a second immunoglobulin light chain variable region that specifically binds to a SARS-CoV-2 protein; wherein VH2 is a second immunoglobulin heavy chain variable region that specifically binds to a SARS-CoV-2 protein; and L1-L4 are amino acid linkers. For example, an antigen binding polypeptide complex provided herein comprises a first polypeptide and a second polypeptide; wherein the first polypeptide has a structure represented by VL1-L1-VH1; and wherein the second polypeptide has a structure represented by VL2-L3-VH2 or VH2-L4-V. For example, an antigen binding polypeptide complex provided herein comprises a first polypeptide and a second polypeptide; wherein the first polypeptide has a structure represented by VH1-L2-VL1; wherein the second polypeptide has a structure represented by VL2-L3-VH2 or VH2-L4-V.

In some aspects, an antigen binding polypeptide complex provided herein comprises a first polypeptide and a second polypeptide; wherein the first polypeptide has a structure represented by VL1-L1-VL2-L2-VH2-L3-VH1; VL1-L1-VH2-L2-VL2-L3-VH1; VH1-L4-VH2-L5-VL2-L6-VL1; or VH1-L4-VL2-L5-VH2-L6-VL1; wherein the second polypeptide has a structure represented by VL3-L7-VL4-L8-VH4-L9-VH3; VL3-L7-VH4-L8-VL4-L9-VH3; VH3-L10-VH4-L11-VL4-L12-VL3; or VH3-L10-VL4-L11-VH4-L12-VL3; wherein VL1 is a first immunoglobulin light chain variable region that specifically binds to a SARS-CoV-2 protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; and L1-L12 are amino acid linkers. In some aspects, the antigen binding polypeptide complex further comprises an Fc region, CH1 region, CL region, or a combination thereof in the first and/or second polypeptides. In some aspects, the antigen binding polypeptide complex comprises an antigen binding polypeptide provided herein. In some aspects, the antigen binding polypeptide complex is an antibody or antigen binding fragment thereof. For example, an antigen binding polypeptide complex provided herein comprises a first polypeptide and a second polypeptide; wherein the first polypeptide has a structure represented by VL1-L1-VL2-L2-VH2-L3-VH1; and wherein the second polypeptide has a structure represented by VL3-L7-VL4-L8-VH4-L9-VH3; VL3-L7-VH4-L8-VL4-L9-VH3; VH3-L10-VH4-L11-VL4-L12-VL3; or VH3-L10-VL4-L11-VH4-L12-VL3. For example, an antigen binding polypeptide complex provided herein comprises a first polypeptide and a second polypeptide; wherein the first polypeptide has a structure represented by VL1-L1-VH2-L2-VL2-L3-VH1; and wherein the second polypeptide has a structure represented by VL3-L7-VL4-L8-VH4-L9-VH3; VL3-L7-VH4-L8-VL4-L9-VH3; VH3-L10-VH4-L11-VL4-L12-VL3; or VH3-L10-VL4-L11-VH4-L12-VL3. For example, an antigen binding polypeptide complex provided herein comprises a first polypeptide and a second polypeptide; wherein the first polypeptide has a structure represented by VH1-L4-VH2-L5-VL2-L6-VL1; and wherein the second polypeptide has a structure represented by VL3-L7-VL4-L8-VH4-L9-VH3; VL3-L7-VH4-L8-VL4-L9-VH3; VH3-L10-VH4-L11-VL4-L12-VL3; or VH3-L10-VL4-L11-VH4-L12-VL3. For example, an antigen binding polypeptide complex provided herein comprises a first polypeptide and a second polypeptide; wherein the first polypeptide has a structure represented by VH1-L4-VL2-L5-VH2-L6-VL1; and wherein the second polypeptide has a structure represented by VL3-L7-VL4-L8-VH4-L9-VH3; VL3-L7-VH4-L8-VL4-L9-VH3; VH3-L10-VH4-L11-VL4-L12-VL3; or VH3-L10-VL4-L11-VH4-L12-VL3. In each example, the first polypeptide may further comprise an Fc region. In each example, the second polypeptide may further comprise an Fc region. In each example, the first polypeptide and the second polypeptide may further comprise an Fc region. Alternatively or in addition, in each example, the first polypeptide may further comprise an CH1 region. In each example, the second polypeptide may further comprise an CH1 region. In each example, the first polypeptide and the second polypeptide may further comprise an CH1 region. Alternatively or in addition, in each example, the first polypeptide may further comprise an CL region. In each example, the second polypeptide may further comprise an CL region. In each example, the first polypeptide and the second polypeptide may further comprise an CL region.

In some aspects, an antigen binding polypeptide complex provided herein comprises a first polypeptide and a second polypeptide; wherein the first polypeptide has a structure represented by VL1-L1-VL2-L2-VH2-L3-VH1 or VH1-L4-VH2-L5-VL2-L6-VL1; wherein the second polypeptide has a structure represented by VL3-L7-VL4-L8-VH4-L9-VH3 or VH3-L10-VH4-L11-VL4-L12-VL3; wherein VL1 is a first immunoglobulin light chain variable region that specifically binds to a SARS-CoV-2 protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; and L1-L12 are amino acid linkers. In some aspects, the antigen binding polypeptide complex further comprises an Fc region, CH1 region, CL region, or a combination thereof in the first and/or second polypeptides. In some aspects, the antigen binding polypeptide complex comprises an antigen binding polypeptide provided herein. In some aspects, the antigen binding polypeptide complex is an antibody or antigen binding fragment thereof. For example, an antigen binding polypeptide complex provided herein comprises a first polypeptide and a second polypeptide; wherein the first polypeptide has a structure represented by VL1-L1-VL2-L2-VH2-L3-VH1; and wherein the second polypeptide has a structure represented by VL3-L7-VL4-L8-VH4-L9-VH3 or VH3-L10-VH4-L11-VL4-L12-VL3. For example, an antigen binding polypeptide complex provided herein comprises a first polypeptide and a second polypeptide; wherein the first polypeptide has a structure represented by VH1-L4-VH2-L5-VL2-L6-VL1; and wherein the second polypeptide has a structure represented by VL3-L7-VL4-L8-VH4-L9-VH3 or VH3-L10-VH4-L11-VL4-L12-VL3. In each example, the first polypeptide may further comprise an Fc region. In each example, the second polypeptide may further comprise an Fc region. In each example, the first polypeptide and the second polypeptide may further comprise an Fc region. Alternatively or in addition, in each example, the first polypeptide may further comprise an CH1 region. In each example, the second polypeptide may further comprise an CH1 region. In each example, the first polypeptide and the second polypeptide may further comprise an CH1 region. Alternatively or in addition, in each example, the first polypeptide may further comprise an CL region. In each example, the second polypeptide may further comprise an CL region. In each example, the first polypeptide and the second polypeptide may further comprise an CL region.

In some aspects, an antigen binding polypeptide complex provided herein comprises a first polypeptide and a second polypeptide; wherein the first polypeptide has a structure represented by VL1-L1-VH2-L2-VL2-L3-VH1 or VH1-L4-VL2-L5-VH2-L6-VL1; wherein the second polypeptide has a structure represented by VL3-L7-VH4-L8-VL4-L9-VH3 or VH3-L10-VL4-L11-VH4-L12-VL3; wherein VL1 is a first immunoglobulin light chain variable region that specifically binds to a SARS-CoV-2 protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; and L1-L12 are amino acid linkers. In some aspects, the antigen binding polypeptide complex further comprises an Fc region, CH1 region, CL region, or a combination thereof in the first and/or second polypeptides. In some aspects, the antigen binding polypeptide complex comprises an antigen binding polypeptide provided herein. In some aspects, the antigen binding polypeptide complex is an antibody or antigen binding fragment thereof. For example, an antigen binding polypeptide complex provided herein comprises a first polypeptide and a second polypeptide; wherein the first polypeptide has a structure represented by VL1-L1-VH2-L2-VL2-L3-VH1; and wherein the second polypeptide has a structure represented by VL3-L7-VH4-L8-VL4-L9-VH3 or VH3-L10-VL4-L11-VH4-L12-VL3. For example, an antigen binding polypeptide complex provided herein comprises a first polypeptide and a second polypeptide; wherein the first polypeptide has a structure represented by VH1-L4-VL2-L5-VH2-L6-VL1; and wherein the second polypeptide has a structure represented by VL3-L7-VH4-L8-VL4-L9-VH3 or VH3-L10-VL4-L11-VH4-L12-VL3. In each example, the first polypeptide may further comprise an Fc region. In each example, the second polypeptide may further comprise an Fc region. In each example, the first polypeptide and the second polypeptide may further comprise an Fc region. Alternatively or in addition, in each example, the first polypeptide may further comprise an CH1 region. In each example, the second polypeptide may further comprise an CH1 region. In each example, the first polypeptide and the second polypeptide may further comprise an CH1 region. Alternatively or in addition, in each example, the first polypeptide may further comprise an CL region. In each example, the second polypeptide may further comprise an CL region. In each example, the first polypeptide and the second polypeptide may further comprise an CL region.

In some aspects, an antigen binding polypeptide provided herein has a structure represented by VL1-L1-VL2-L2-VH2-L3-VH1-L4-Fc; VL1-L1-VH2-L2-VL2-L3-VH1-L4-Fc; VH1-L5-VH2-L6-VL2-L7-VL1-L8-Fc; or VH1-L5-VL2-L6-VH2-L7-VL1-L8-Fc; wherein VL1 is a first immunoglobulin light chain variable region that specifically binds to a SARS-CoV-2 protein; VL2 is a second immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; Fc is a region comprising an immunoglobulin heavy chain constant region 2 (CH2), an immunoglobulin heavy chain constant region 3 (CH3), and optionally, an immunoglobulin hinge; and L1-L8 are amino acid linkers. In some aspects, the antigen binding polypeptide further comprises a CH1 region, CL region, or a combination thereof. In some aspects, the antigen binding polypeptide is part of an antigen binding polypeptide complex provided herein (e.g., an antibody or antigen binding fragment thereof).

In some aspects, an antigen binding polypeptide provided herein has a structure represented by VL1-L1-VL2-L2-VH2-L3-VH1-L4-Fc or VH1-L5-VH2-L6-VL2-L7-VL1-L8-Fc; wherein VL1 is a first immunoglobulin light chain variable region that specifically binds to a SARS-CoV-2 protein; VL2 is a second immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; Fc is a region comprising an immunoglobulin heavy chain constant region 2 (CH2), an immunoglobulin heavy chain constant region 3 (CH3), and optionally, an immunoglobulin hinge; and L1-L8 are amino acid linkers. In some aspects, the antigen binding polypeptide further comprises a CH1 region, CL region, or a combination thereof. In some aspects, the antigen binding polypeptide is part of an antigen binding polypeptide complex provided herein (e.g., an antibody or antigen binding fragment thereof).

In some aspects, an antigen binding polypeptide provided herein has a structure represented by VL1-L1-VH2-L2-VL2-L3-VH1-L4-Fc or VH1-L5-VL2-L6-VH2-L7-VL1-L8-Fc; wherein VL1 is a first immunoglobulin light chain variable region that specifically binds to a SARS-CoV-2 protein; VL2 is a second immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; Fc is a region comprising an immunoglobulin heavy chain constant region 2 (CH2), an immunoglobulin heavy chain constant region 3 (CH3), and optionally, an immunoglobulin hinge; and L1-L8 are amino acid linkers. In some aspects, the antigen binding polypeptide further comprises a CH1 region, CL region, or a combination thereof. In some aspects, the antigen binding polypeptide is part of an antigen binding polypeptide complex provided herein (e.g., an antibody or antigen binding fragment thereof).

In some aspects, an antigen binding polypeptide complex provided herein comprises a first polypeptide and a second polypeptide; wherein the first polypeptide has a structure represented by VL1-L1-VL2-L2-VH2-L3-VH1-L4-Fc; VL1-L1-VH2-L2-VL2-L3-VH1-L4-Fc; VH1-L5-VH2-L6-VL2-L7-VL1-L8-Fc; or VH1-L5-VL2-L6-VH2-L7-VL1-L8-Fc; wherein the second polypeptide has a structure represented by Fc; VL3-L9-VL4-L10-VH4-L11-VH3-L12-Fc; VL3-L9-VH4-L10-VL4-L11-VH3-L12-Fc; VH3-L13-VH4-L14-VL4-L15-VL3-L16-Fc; or VH3-L13-VL4-L14-VH4-L15-VL3-L16-Fc; wherein VL1 is a first immunoglobulin light chain variable region that specifically binds to a SARS-CoV-2 protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; Fc is a region comprising an immunoglobulin heavy chain constant region 2 (CH2), an immunoglobulin heavy chain constant region 3 (CH3), and optionally, an immunoglobulin hinge; and L1-L16 are amino acid linkers. In some aspects, the antigen binding polypeptide complex further comprises a CH1 region, CL region, or a combination thereof in the first and/or second polypeptides. In some aspects, the antigen binding polypeptide complex comprises an antigen binding polypeptide provided herein. In some aspects, the antigen binding polypeptide complex is an antibody or antigen binding fragment thereof. For example, an antigen binding polypeptide complex provided herein comprises a first polypeptide and a second polypeptide; wherein the first polypeptide has a structure represented by VL1-L1-VL2-L2-VH2-L3-VH1-L4-Fc; and wherein the second polypeptide has a structure represented by Fc; VL3-L9-VL4-L10-VH4-L11-VH3-L12-Fc; VL3-L9-VH4-L10-VL4-L11-VH3-L12-Fc; VH3-L13-VH4-L14-VL4-L15-VL3-L16-Fc; or VH3-L13-VL4-L14-VH4-L15-VL3-L16-Fc. For example, an antigen binding polypeptide complex provided herein comprises a first polypeptide and a second polypeptide; wherein the first polypeptide has a structure represented by VL1-L1-VH2-L2-VL2-L3-VH1-L4-Fc; and wherein the second polypeptide has a structure represented by Fc; VL3-L9-VL4-L10-VH4-L11-VH3-L12-Fc; VL3-L9-VH4-L10-VL4-L11-VH3-L12-Fc; VH3-L13-VH4-L14-VL4-L15-VL3-L16-Fc; or VH3-L13-VL4-L14-VH4-L15-VL3-L16-Fc. For example, an antigen binding polypeptide complex provided herein comprises a first polypeptide and a second polypeptide; wherein the first polypeptide has a structure represented by VH1-L5-VH2-L6-VL2-L7-VL1-L8-Fc; and wherein the second polypeptide has a structure represented by Fc; VL3-L9-VL4-L10-VH4-L11-VH3-L12-Fc; VL3-L9-VH4-L10-VL4-L11-VH3-L12-Fc; VH3-L13-VH4-L14-VL4-L15-VL3-L16-Fc; or VH3-L13-VL4-L14-VH4-L15-VL3-L16-Fc. For example, an antigen binding polypeptide complex provided herein comprises a first polypeptide and a second polypeptide; wherein the first polypeptide has a structure represented by VH1-L5-VL2-L6-VH2-L7-VL1-L8-Fc; and wherein the second polypeptide has a structure represented by Fc; VL3-L9-VL4-L10-VH4-L11-VH3-L12-Fc; VL3-L9-VH4-L10-VL4-L11-VH3-L12-Fc; VH3-L13-VH4-L14-VL4-L15-VL3-L16-Fc; or VH3-L13-VL4-L14-VH4-L15-VL3-L16-Fc. In each example, the first polypeptide may further comprise an CH1 region. In each example, the second polypeptide may further comprise an CH1 region. In each example, the first polypeptide and the second polypeptide may further comprise an CH1 region. Alternatively or in addition, in each example, the first polypeptide may further comprise an CL region. In each example, the second polypeptide may further comprise an CL region. In each example, the first polypeptide and the second polypeptide may further comprise an CL region.

In some aspects, an antigen binding polypeptide complex provided herein comprises a first polypeptide and a second polypeptide; wherein the first polypeptide has a structure represented by VL1-L1-VL2-L2-VH2-L3-VH1-L4-Fc or VH1-L5-VH2-L6-VL2-L7-VL1-L8-Fc; wherein the second polypeptide has a structure represented by Fc; VL3-L9-VL4-L10-VH4-L11-VH3-L12-Fc; or VH3-L13-VH4-L14-VL4-L15-VL3-L16-Fc; wherein VL1 is a first immunoglobulin light chain variable region that specifically binds to a SARS-CoV-2 protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; Fc is a region comprising an immunoglobulin heavy chain constant region 2 (CH2), an immunoglobulin heavy chain constant region 3 (CH3), and optionally, an immunoglobulin hinge; and L1-L16 are amino acid linkers. In some aspects, the antigen binding polypeptide complex further comprises a CH1 region, CL region, or a combination thereof in the first and/or second polypeptides. In some aspects, the antigen binding polypeptide complex comprises an antigen binding polypeptide provided herein. In some aspects, the antigen binding polypeptide complex is an antibody or antigen binding fragment thereof. For example, an antigen binding polypeptide complex provided herein comprises a first polypeptide and a second polypeptide; wherein the first polypeptide has a structure represented by VL1-L1-VL2-L2-VH2-L3-VH1-L4-Fc; and wherein the second polypeptide has a structure represented by Fc; VL3-L9-VL4-L10-VH4-L11-VH3-L12-Fc; or VH3-L13-VH4-L14-VL4-L15-VL3-L16-Fc. For example, an antigen binding polypeptide complex provided herein comprises a first polypeptide and a second polypeptide; wherein the first polypeptide has a structure represented by VH1-L5-VH2-L6-VL2-L7-VL1-L8-Fc; and wherein the second polypeptide has a structure represented by Fc; VL3-L9-VL4-L10-VH4-L11-VH3-L12-Fc; or VH3-L13-VH4-L14-VL4-L15-VL3-L16-Fc. In each example, the first polypeptide may further comprise an CH1 region. In each example, the second polypeptide may further comprise an CH1 region. In each example, the first polypeptide and the second polypeptide may further comprise an CH1 region. Alternatively or in addition, in each example, the first polypeptide may further comprise an CL region. In each example, the second polypeptide may further comprise an CL region. In each example, the first polypeptide and the second polypeptide may further comprise an CL region.

In some aspects, an antigen binding polypeptide complex provided herein comprises a first polypeptide and a second polypeptide; wherein the first polypeptide has a structure represented by VL1-L1-VH2-L2-VL2-L3-VH1-L4-Fc or VH1-L5-VL2-L6-VH2-L7-VL1-L8-Fc; wherein the second polypeptide has a structure represented by Fc; VL3-L9-VH4-L10-VL4-L11-VH3-L12-Fc; or VH3-L13-VL4-L14-VH4-L15-VL3-L16-Fc; wherein VL1 is a first immunoglobulin light chain variable region that specifically binds to a SARS-CoV-2 protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; Fc is a region comprising an immunoglobulin heavy chain constant region 2 (CH2), an immunoglobulin heavy chain constant region 3 (CH3), and optionally, an immunoglobulin hinge; and L1-L16 are amino acid linkers. In some aspects, the antigen binding polypeptide complex further comprises a CH1 region, CL region, or a combination thereof in the first and/or second polypeptides. In some aspects, the antigen binding polypeptide complex comprises an antigen binding polypeptide provided herein. In some aspects, the antigen binding polypeptide complex is an antibody or antigen binding fragment thereof. For example, an antigen binding polypeptide complex provided herein comprises a first polypeptide and a second polypeptide; wherein the first polypeptide has a structure represented by VL1-L1-VH2-L2-VL2-L3-VH1-L4-Fc; wherein the second polypeptide has a structure represented by Fc; VL3-L9-VH4-L10-VL4-L11-VH3-L12-Fc; or VH3-L13-VL4-L14-VH4-L15-VL3-L16-Fc. For example, an antigen binding polypeptide complex provided herein comprises a first polypeptide and a second polypeptide; wherein the first polypeptide has a structure represented by VH1-L5-VL2-L6-VH2-L7-VL1-L8-Fc; wherein the second polypeptide has a structure represented by Fc; VL3-L9-VH4-L10-VL4-L11-VH3-L12-Fc; or VH3-L13-VL4-L14-VH4-L15-VL3-L16-Fc. In each example, the first polypeptide may further comprise an CH1 region. In each example, the second polypeptide may further comprise an CH1 region. In each example, the first polypeptide and the second polypeptide may further comprise an CH1 region. Alternatively or in addition, in each example, the first polypeptide may further comprise an CL region. In each example, the second polypeptide may further comprise an CL region. In each example, the first polypeptide and the second polypeptide may further comprise an CL region.

In some aspects, an antigen binding polypeptide provided herein has a structure represented by VL1-L1-VL2-L2-VH2-L3-VH1-L4-CH1-L5-CL; VL1-L1-VH2-L2-VL2-L3-VH1-L4-CH1-L5-CL; VH1-L6-VH2-L7-VL2-L8-VL1-L9-CH1-L10-CL; VH1-L6-VL2-L7-VH2-L8-VL1-L9-CH1-L10-CL; VL1-L11-VL2-L12-VH2-L13-VH1-L14-CL-L15-CH1; VL1-L11-VH2-L12-VL2-L13-VH1-L14-CL-L15-CH1; VH1-L16-VH2-L17-VL2-L18-VL1-L19-CL-L20-CH1; or VH1-L16-VL2-L17-VH2-L18-VL1-L19-CL-L20-CH1; wherein VL1 is a first immunoglobulin light chain variable region that specifically binds to a SARS-CoV-2 protein; VL2 is a second immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; CH1 is an immunoglobulin heavy chain constant region 1; CL is an immunoglobulin light chain constant region; and L1-L20 are amino acid linkers. In some aspects, the antigen binding polypeptide further comprises an Fc region. In some aspects, the antigen binding polypeptide is part of an antigen binding polypeptide complex provided herein (e.g., an antibody or antigen binding fragment thereof).

In some aspects, an antigen binding polypeptide provided herein has a structure represented by VL1-L1-VL2-L2-VH2-L3-VH1-L4-CH1-L5-CL; VH1-L6-VH2-L7-VL2-L8-VL1-L9-CH1-L10-CL; VL1-L11-VL2-L12-VH2-L13-VH1-L14-CL-L15-CH1; or VH1-L16-VH2-L17-VL2-L18-VL1-L19-CL-L20-CH1; wherein VL1 is a first immunoglobulin light chain variable region that specifically binds to a SARS-CoV-2 protein; VL2 is a second immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; CH1 is an immunoglobulin heavy chain constant region 1; CL is an immunoglobulin light chain constant region; and L1-L20 are amino acid linkers. In some aspects, the antigen binding polypeptide further comprises an Fc region. In some aspects, the antigen binding polypeptide is part of an antigen binding polypeptide complex provided herein (e.g., an antibody or antigen binding fragment thereof).

In some aspects, an antigen binding polypeptide provided herein has a structure represented by VL1-L1-VH2-L2-VL2-L3-VH1-L4-CH1-L5-CL; VH1-L6-VL2-L7-VH2-L8-VL1-L9-CH1-L10-CL; VL1-L11-VH2-L12-VL2-L13-VH1-L14-CL-L15-CH1; or VH1-L16-VL2-L17-VH2-L18-VL1-L19-CL-L20-CH1; wherein VL1 is a first immunoglobulin light chain variable region that specifically binds to a SARS-CoV-2 protein; VL2 is a second immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; CH1 is an immunoglobulin heavy chain constant region 1; CL is an immunoglobulin light chain constant region; and L1-L20 are amino acid linkers. In some aspects, the antigen binding polypeptide further comprises an Fc region. In some aspects, the antigen binding polypeptide is part of an antigen binding polypeptide complex provided herein (e.g., an antibody or antigen binding fragment thereof).

In some aspects, an antigen binding polypeptide complex provided herein comprises a first polypeptide and a second polypeptide; wherein the first polypeptide has a structure represented by: VL1-L1-VL2-L2-VH2-L3-VH1-L4-CH1-L5-CL; VL1-L1-VH2-L2-VL2-L3-VH1-L4-CH1-L5-CL; VH1-L6-VH2-L7-VL2-L8-VL1-L9-CH1-L10-CL; VH1-L6-VL2-L7-VH2-L8-VL1-L9-CH1-L10-CL; VL1-L11-VL2-L12-VH2-L13-VH1-L14-CL-L15-CH1; VL1-L11-VH2-L12-VL2-L13-VH1-L14-CL-L15-CH1; VH1-L16-VH2-L17-VL2-L18-VL1-L19-CL-L20-CH1; or VH1-L16-VL2-L17-VH2-L18-VL1-L19-CL-L20-CH1; wherein the second polypeptide has a structure represented by VL3-L21-VL4-L22-VH4-L23-VH3-L24-CH1-L25-CL; VL3-L21-VH4-L22-VL4-L23-VH3-L24-CH1-L25-CL; VH3-L26-VH4-L27-VL4-L28-VL3-L29-CH1-L30-CL; VH3-L26-VL4-L27-VH4-L28-VL3-L29-CH1-L30-CL; VL3-L31-VL4-L32-VH4-L33-VH3-L34-CL-L35-CH1; VL3-L31-VH4-L32-VL4-L33-VH3-L34-CL-L35-CH1; VH3-L36-VH4-L37-VL4-L38-VL3-L39-CL-L40-CH1; or VH3-L36-VL4-L37-VH4-L38-VL3-L39-CL-L40-CH1; wherein VL1 is a first immunoglobulin light chain variable region that specifically binds to a SARS-CoV-2 protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; CH1 is an immunoglobulin heavy chain constant region 1; CL is an immunoglobulin light chain constant region; and L1-L40 are amino acid linkers. In some aspects, the antigen binding polypeptide complex further comprises an Fc region in the first and/or second polypeptides. In some aspects, the antigen binding polypeptide complex comprises an antigen binding polypeptide provided herein. In some aspects, the antigen binding polypeptide complex is an antibody or antigen binding fragment thereof. Any combination of VH1, VH2, VH3 and/or VH4 described above may be used in combination with any combination of VL1, VL2, VL3 and/or VL4 described above. In particular, any first polypeptide described above may be combined with any second polypeptide described above to form an antigen binding polypeptide complex.

In some aspects, an antigen binding polypeptide complex provided herein comprises a first polypeptide and a second polypeptide; wherein the first polypeptide has a structure represented by VL1-L1-VL2-L2-VH2-L3-VH1-L4-CH1-L5-CL; VH1-L6-VH2-L7-VL2-L8-VL1-L9-CH1-L10-CL; VL1-L11-VL2-L12-VH2-L13-VH1-L14-CL-L15-CH1; or VH1-L16-VH2-L17-VL2-L18-VL1-L19-CL-L20-CH1; wherein the second polypeptide has a structure represented by VL3-L21-VL4-L22-VH4-L23-VH3-L24-CH1-L25-CL; VH3-L26-VH4-L27-VL4-L28-VL3-L29-CH1-L30-CL; VL3-L31-VL4-L32-VH4-L33-VH3-L34-CL-L35-CH1; or VH3-L36-VH4-L37-VL4-L38-VL3-L39-CL-L40-CH1; wherein VL1 is a first immunoglobulin light chain variable region that specifically binds to a SARS-CoV-2 protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; CH1 is an immunoglobulin heavy chain constant region 1; CL is an immunoglobulin light chain constant region; and L1-L40 are amino acid linkers. In some aspects, the antigen binding polypeptide complex further comprises an Fc region in the first and/or second polypeptides. In some aspects, the antigen binding polypeptide complex comprises an antigen binding polypeptide provided herein. In some aspects, the antigen binding polypeptide complex is an antibody or antigen binding fragment thereof. Any combination of VH1, VH2, VH3 and/or VH4 described above may be used in combination with any combination of VL1, VL2, VL3 and/or VL4 described above. For example, an antigen binding polypeptide complex provided herein comprises a first polypeptide and a second polypeptide; wherein the first polypeptide has a structure represented by VL1-L1-VL2-L2-VH2-L3-VH1-L4-CH1-L5-CL; wherein the second polypeptide has a structure represented by VL3-L21-VL4-L22-VH4-L23-VH3-L24-CH1-L25-CL; VH3-L26-VH4-L27-VL4-L28-VL3-L29-CH1-L30-CL; VL3-L31-VL4-L32-VH4-L33-VH3-L34-CL-L35-CH1; or VH3-L36-VH4-L37-VL4-L38-VL3-L39-CL-L40-CH1. For example, an antigen binding polypeptide complex provided herein comprises a first polypeptide and a second polypeptide; wherein the first polypeptide has a structure represented by VH1-L6-VH2-L7-VL2-L8-VL1-L9-CH1-L10-CL; wherein the second polypeptide has a structure represented by VL3-L21-VL4-L22-VH4-L23-VH3-L24-CH1-L25-CL; VH3-L26-VH4-L27-VL4-L28-VL3-L29-CH1-L30-CL; VL3-L31-VL4-L32-VH4-L33-VH3-L34-CL-L35-CH1; or VH3-L36-VH4-L37-VL4-L38-VL3-L39-CL-L40-CH1. VH1-L6-VH2-L7-VL2-L8-VL1-L9-CH1-L10-CLVH1-L6-VH2-L7-VL2-L8-VL1-L9-CH1-L10-CL. For example, an antigen binding polypeptide complex provided herein comprises a first polypeptide and a second polypeptide; wherein the first polypeptide has a structure represented by VL1-L11-VL2-L12-VH2-L13-VH1-L14-CL-L15-CH1; wherein the second polypeptide has a structure represented by VL3-L21-VL4-L22-VH4-L23-VH3-L24-CH1-L25-CL; VH3-L26-VH4-L27-VL4-L28-VL3-L29-CH1-L30-CL; VL3-L31-VL4-L32-VH4-L33-VH3-L34-CL-L35-CH1; or VH3-L36-VH4-L37-VL4-L38-VL3-L39-CL-L40-CH1. VH1-L6-VH2-L7-VL2-L8-VL1-L9-CH1-L10-CLVH1-L6-VH2-L7-VL2-L8-VL1-L9-CH1-L10-CL. For example, an antigen binding polypeptide complex provided herein comprises a first polypeptide and a second polypeptide; wherein the first polypeptide has a structure represented by VH1-L16-VH2-L17-VL2-L18-VL1-L19-CL-L20-CH1; wherein the second polypeptide has a structure represented by VL3-L21-VL4-L22-VH4-L23-VH3-L24-CH1-L25-CL; VH3-L26-VH4-L27-VL4-L28-VL3-L29-CH1-L30-CL; VL3-L31-VL4-L32-VH4-L33-VH3-L34-CL-L35-CH1; or VH3-L36-VH4-L37-VL4-L38-VL3-L39-CL-L40-CH1. VH1-L6-VH2-L7-VL2-L8-VL1-L9-CH1-L10-CLVH1-L6-VH2-L7-VL2-L8-VL1-L9-CH1-L10-CL.

In some aspects, an antigen binding polypeptide complex provided herein comprises a first polypeptide and a second polypeptide; wherein the first polypeptide has a structure represented by VL1-L1-VH2-L2-VL2-L3-VH1-L4-CH1-L5-CL; VH1-L6-VL2-L7-VH2-L8-VL1-L9-CH1-L10-CL; VL1-L11-VH2-L12-VL2-L13-VH1-L14-CL-L15-CH1; or VH1-L16-VL2-L17-VH2-L18-VL1-L19-CL-L20-CH1; wherein the second polypeptide has a structure represented by VL3-L21-VH4-L22-VL4-L23-VH3-L24-CH1-L25-CL; VH3-L26-VL4-L27-VH4-L28-VL3-L29-CH1-L30-CL; VL3-L31-VH4-L32-VL4-L33-VH3-L34-CL-L35-CH1; or VH3-L36-VL4-L37-VH4-L38-VL3-L39-CL-L40-CH1; wherein VL1 is a first immunoglobulin light chain variable region that specifically binds to a SARS-CoV-2 protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; CH1 is an immunoglobulin heavy chain constant region 1; CL is an immunoglobulin light chain constant region; and L1-L40 are amino acid linkers. In some aspects, the antigen binding polypeptide complex further comprises an Fc region in the first and/or second polypeptides. In some aspects, the antigen binding polypeptide complex comprises an antigen binding polypeptide provided herein. In some aspects, the antigen binding polypeptide complex is an antibody or antigen binding fragment thereof. Any combination of VH1, VH2, VH3 and/or VH4 described above may be used in combination with any combination of VL1, VL2, VL3 and/or VL4 described above. In particular, any first polypeptide described above may be combined with any second polypeptide described above to form an antigen binding polypeptide complex.

In some aspects, an antigen binding polypeptide provided herein has a structure represented by VL1-L1-VL2-L2-VH2-L3-VH1-L4-CH1-L5-CL-L6-Fc; VL1-L1-VH2-L2-VL2-L3-VH1-L4-CH1-L5-CL-L6-Fc; VH1-L7-VH2-L8-VL2-L9-VL1-L10-CH1-L11-CL-L12-Fc; VH1-L7-VL2-L8-VH2-L9-VL1-L10-CH1-L11-CL-L12-Fc; VL1-L13-VL2-L14-VH2-L15-VH1-L16-CL-L17-CH1-L18-Fc; VL1-L13-VH2-L14-VL2-L15-VH1-L16-CL-L17-CH1-L18-Fc; VH1-L19-VH2-L20-VL2-L21-VL1-L22-CL-L23-CH1-L24-Fc; or VH1-L19-VL2-L20-VH2-L21-VL1-L22-CL-L23-CH1-L24-Fc; wherein VL1 is a first immunoglobulin light chain variable region that specifically binds to a SARS-CoV-2 protein; VL2 is a second immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; CH1 is an immunoglobulin heavy chain constant region 1; CL is an immunoglobulin light chain constant region; Fc is a region comprising an immunoglobulin heavy chain constant region 2 (CH2), an immunoglobulin heavy chain constant region 3 (CH3), and optionally, an immunoglobulin hinge; and L1-L24 are amino acid linkers. In some aspects, the antigen binding polypeptide is part of an antigen binding polypeptide complex provided herein (e.g., an antibody or antigen binding fragment thereof).

In some aspects, an antigen binding polypeptide provided herein has a structure represented by VL1-L1-VL2-L2-VH2-L3-VH1-L4-CH1-L5-CL-L6-Fc; VH1-L7-VH2-L8-VL2-L9-VL1-L10-CH1-L11-CL-L12-Fc; VL1-L13-VL2-L14-VH2-L15-VH1-L16-CL-L17-CH1-L18-Fc; or VH1-L19-VH2-L20-VL2-L21-VL1-L22-CL-L23-CH1-L24-Fc; wherein VL1 is a first immunoglobulin light chain variable region that specifically binds to a SARS-CoV-2 protein; VL2 is a second immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; CH1 is an immunoglobulin heavy chain constant region 1; CL is an immunoglobulin light chain constant region; Fc is a region comprising an immunoglobulin heavy chain constant region 2 (CH2), an immunoglobulin heavy chain constant region 3 (CH3), and optionally, an immunoglobulin hinge; and L1-L24 are amino acid linkers. In some aspects, the antigen binding polypeptide is part of an antigen binding polypeptide complex provided herein (e.g., an antibody or antigen binding fragment thereof).

In some aspects, an antigen binding polypeptide provided herein has a structure represented by VL1-L1-VH2-L2-VL2-L3-VH1-L4-CH1-L5-CL-L6-Fc; VH1-L7-VL2-L8-VH2-L9-VL1-L10-CH1-L11-CL-L12-Fc; VL1-L13-VH2-L14-VL2-L15-VH1-L16-CL-L17-CH1-L18-Fc; or VH1-L19-VL2-L20-VH2-L21-VL1-L22-CL-L23-CH1-L24-Fc; wherein VL1 is a first immunoglobulin light chain variable region that specifically binds to a SARS-CoV-2 protein; VL2 is a second immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; CH1 is an immunoglobulin heavy chain constant region 1; CL is an immunoglobulin light chain constant region; Fc is a region comprising an immunoglobulin heavy chain constant region 2 (CH2), an immunoglobulin heavy chain constant region 3 (CH3), and optionally, an immunoglobulin hinge; and L1-L24 are amino acid linkers. In some aspects, the antigen binding polypeptide is part of an antigen binding polypeptide complex provided herein (e.g., an antibody or antigen binding fragment thereof).

In some aspects, an antigen binding polypeptide complex provided herein comprises a first polypeptide and a second polypeptide; wherein the first polypeptide has a structure represented by VL1-L1-VL2-L2-VH2-L3-VH1-L4-CH1-L5-CL-L6-Fc; VL1-L1-VH2-L2-VL2-L3-VH1-L4-CH1-L5-CL-L6-Fc; VH1-L7-VH2-L8-VL2-L9-VL1-L10-CH1-L11-CL-L12-Fc; VH1-L7-VL2-L8-VH2-L9-VL1-L10-CH1-L11-CL-L12-Fc; VL1-L13-VL2-L14-VH2-L15-VH1-L16-CL-L17-CH1-L18-Fc; VL1-L13-VH2-L14-VL2-L15-VH1-L16-CL-L17-CH1-L18-Fc; VH1-L19-VH2-L20-VL2-L21-VL1-L22-CL-L23-CH1-L24-Fc; or VH1-L19-VL2-L20-VH2-L21-VL1-L22-CL-L23-CH1-L24-Fc; wherein the second polypeptide has a structure represented by Fc; VL3-L25-VL4-L26-VH4-L27-VH3-L28-CH1-L29-CL-L30-Fc; VL3-L25-VH4-L26-VL4-L27-VH3-L28-CH1-L29-CL-L30-Fc; VH3-L31-VH4-L32-VL4-L33-VL3-L34-CH1-L35-CL-L36-Fc; VH3-L31-VL4-L32-VH4-L33-VL3-L34-CH1-L35-CL-L36-Fc; VL3-L37-VL4-L38-VH4-L39-VH3-L40-CL-L41-CH1-L42-Fc; VL3-L37-VH4-L38-VL4-L39-VH3-L40-CL-L41-CH1-L42-Fc; VH3-L43-VH4-L44-VL4-L45-VL3-L46-CL-L47-CH1-L48-Fc; or VH3-L43-VL4-L44-VH4-L45-VL3-L46-CL-L47-CH1-L48-Fc; wherein VL1 is a first immunoglobulin light chain variable region that specifically binds to a SARS-CoV-2 protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; CH1 is an immunoglobulin heavy chain constant region 1; CL is an immunoglobulin light chain constant region; Fc is a region comprising an immunoglobulin heavy chain constant region 2 (CH2), an immunoglobulin heavy chain constant region 3 (CH3), and optionally, an immunoglobulin hinge; and L1-L48 are amino acid linkers. In some aspects, the antigen binding polypeptide complex comprises an antigen binding polypeptide provided herein. In some aspects, the antigen binding polypeptide complex is an antibody or antigen binding fragment thereof. Any combination of VH1, VH2, VH3 and/or VH4 described above may be used in combination with any combination of VL1, VL2, VL3 and/or VL4 described above. In particular, any first polypeptide described above may be combined with any second polypeptide described above to form an antigen binding polypeptide complex.

In some aspects, an antigen binding polypeptide complex provided herein comprises a first polypeptide and a second polypeptide; wherein the first polypeptide has a structure represented by VL1-L1-VL2-L2-VH2-L3-VH1-L4-CH1-L5-CL-L6-Fc; VH1-L7-VH2-L8-VL2-L9-VL1-L10-CH1-L11-CL-L12-Fc; VL1-L13-VL2-L14-VH2-L15-VH1-L16-CL-L17-CH1-L18-Fc; or VH1-L19-VH2-L20-VL2-L21-VL1-L22-CL-L23-CH1-L24-Fc; wherein the second polypeptide has a structure represented by Fc; VL3-L25-VL4-L26-VH4-L27-VH3-L28-CH1-L29-CL-L30-Fc; VH3-L31-VH4-L32-VL4-L33-VL3-L34-CH1-L35-CL-L36-Fc; VL3-L37-VL4-L38-VH4-L39-VH3-L40-CL-L41-CH1-L42-Fc; or VH3-L43-VH4-L44-VL4-L45-VL3-L46-CL-L47-CH1-L48-Fc; wherein VL1 is a first immunoglobulin light chain variable region that specifically binds to a SARS-CoV-2 protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; CH1 is an immunoglobulin heavy chain constant region 1; CL is an immunoglobulin light chain constant region; Fc is a region comprising an immunoglobulin heavy chain constant region 2 (CH2), an immunoglobulin heavy chain constant region 3 (CH3), and optionally, an immunoglobulin hinge; and L1-L48 are amino acid linkers. In some aspects, the antigen binding polypeptide complex comprises an antigen binding polypeptide provided herein. In some aspects, the antigen binding polypeptide complex is an antibody or antigen binding fragment thereof. Any combination of VH1, VH2, VH3 and/or VH4 described above may be used in combination with any combination of VL1, VL2, VL3 and/or VL4 described above. In particular, any first polypeptide described above may be combined with any second polypeptide described above to form an antigen binding polypeptide complex.

In some aspects, an antigen binding polypeptide complex provided herein comprises a first polypeptide and a second polypeptide; wherein the first polypeptide has a structure represented by VL1-L1-VH2-L2-VL2-L3-VH1-L4-CH1-L5-CL-L6-Fc; VH1-L7-VL2-L8-VH2-L9-VL1-L10-CH1-L11-CL-L12-Fc; VL1-L13-VH2-L14-VL2-L15-VH1-L16-CL-L17-CH1-L18-Fc; or VH1-L19-VL2-L20-VH2-L21-VL1-L22-CL-L23-CH1-L24-Fc; wherein the second polypeptide has a structure represented by Fc; VL3-L25-VH4-L26-VL4-L27-VH3-L28-CH1-L29-CL-L30-Fc; VH3-L31-VL4-L32-VH4-L33-VL3-L34-CH1-L35-CL-L36-Fc; VL3-L37-VH4-L38-VL4-L39-VH3-L40-CL-L41-CH1-L42-Fc; or VH3-L43-VL4-L44-VH4-L45-VL3-L46-CL-L47-CH1-L48-Fc; wherein VL1 is a first immunoglobulin light chain variable region that specifically binds to a SARS-CoV-2 protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; CH1 is an immunoglobulin heavy chain constant region 1; CL is an immunoglobulin light chain constant region; Fc is a region comprising an immunoglobulin heavy chain constant region 2 (CH2), an immunoglobulin heavy chain constant region 3 (CH3), and optionally, an immunoglobulin hinge; and L1-L48 are amino acid linkers. In some aspects, the antigen binding polypeptide complex comprises an antigen binding polypeptide provided herein. In some aspects, the antigen binding polypeptide complex is an antibody or antigen binding fragment thereof. Any combination of VH1, VH2, VH3 and/or VH4 described above may be used in combination with any combination of VL1, VL2, VL3 and/or VL4 described above. In particular, any first polypeptide described above may be combined with any second polypeptide described above to form an antigen binding polypeptide complex.

In some aspects, one or more of VH1, VH2, VH3 and VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666, or any other CDR sequence within a larger VH or full length polypeptide chain sequence disclosed herein. In some preferred aspects, CDR1, CDR2 and CDR3 of one or more of VH1, VH2, VH3 and VH4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of one or more of VH1, VH2, VH3 and VH4 comprise or consist of the corresponding amino acid sequences specified above. In some preferred aspects, CDR1, CDR2 and CDR3 of VH1, VH2, VH3 and VH4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3 and VH4 comprise or consist of the corresponding amino acid sequences specified above.

In some aspects, one or more of VL1, VL2, VL3 and VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:39, 47, 55, 63, 71, 167, 175, 183, 260, 268, 276, 284, 517, 525, 533, 541, 549, 557, 565, 573, 581, 589, 597, 605, 613, 621, 643, 651, 660 and 668; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:40, 48, 56, 64, 72, 168, 176, 184, 261, 269, 277 (having the sequence of LGS), 285, 518 (having the sequence of DAS), 526 (having the sequence of DVS), 534 (having the sequence of EDS), 542 (having the sequence of KDS), 550 (having the sequence of DAS), 558 (having the sequence of AAS), 566 (having the sequence of GAS), 574 (having the sequence of DDS), 582 (having the sequence of KDS), 590 (having the sequence of SAS), 598 (having the sequence of SAS), 606 (having the sequence of GAS), 614 (having the sequence of GAS), 622 (having the sequence of GAS), 644 (having the sequence of GAS), 652 (having the sequence of SAS), 661 (having the sequence of GAS) and 669 (having the sequence of DAS); and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:41, 49, 57, 65, 73, 169, 177, 185, 262, 270, 278, 286, 519, 527, 535, 543, 551, 559, 567, 575, 583, 591, 599, 607, 615, 623, 645, 653, 662 and 670, or any other CDR sequence within a larger VL or full length polypeptide chain sequence disclosed herein. In some preferred aspects, CDR1, CDR2 and CDR3 of one or more of VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of one or more of VL1, VL2, VL3 and VL4 comprise or consist of the corresponding amino acid sequences specified above. In some preferred aspects, CDR1, CDR2 and CDR3 of VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VL1, VL2, VL3 and VL4 comprise or consist of the corresponding amino acid sequences specified above.

In some aspects, one or more of VH1, VH2, VH3 and VH4 comprises a CDR1 comprising an amino acid sequence having at least 80%, at least 85%, at least 90%, at least 91%, at least 92%, at least 93%, at least 94%, at least 95%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666; and one or more of VL1, VL2, VL3 and VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:39, 47, 55, 63, 71, 167, 175, 183, 260, 268, 276, 284, 517, 525, 533, 541, 549, 557, 565, 573, 581, 589, 597, 605, 613, 621, 643, 651, 660 and 668; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:40, 48, 56, 64, 72, 168, 176, 184, 261, 269, 277 (having the sequence of LGS), 285, 518 (having the sequence of DAS), 526 (having the sequence of DVS), 534 (having the sequence of EDS), 542 (having the sequence of KDS), 550 (having the sequence of DAS), 558 (having the sequence of AAS), 566 (having the sequence of GAS), 574 (having the sequence of DDS), 582 (having the sequence of KDS), 590 (having the sequence of SAS), 598 (having the sequence of SAS), 606 (having the sequence of GAS), 614 (having the sequence of GAS), 622 (having the sequence of GAS), 644 (having the sequence of GAS), 652 (having the sequence of SAS), 661 (having the sequence of GAS) and 669 (having the sequence of DAS); and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:41, 49, 57, 65, 73, 169, 177, 185, 262, 270, 278, 286, 591, 599, 607, 615, 623, 645, 653, 662 and 670, or any other CDR sequence within a larger VH, VL or full length polypeptide chain sequence disclosed herein. In some preferred aspects, CDR1, CDR2 and CDR3 of one or more of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of one or more of VH1, VH2, VH3, VH4, VL1, VL2, VL3 and VL4 comprise or consist of the corresponding amino acid sequences specified above. Any combination of VH1, VH2, VH3 and/or VH4 described above may be used in combination with any combination of VL1, VL2, VL3 and/or VL4 described above. In some preferred aspects, CDR1, CDR2 and CDR3 of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, VH4, VL1, VL2, VL3 and VL4 comprise or consist of the corresponding amino acid sequences specified above.

In some aspects, one or more of VH1, VH2, VH3 and VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:42, 50, 58, 66, 74, 170, 178, 186, 263, 271, 279, 287, 510, 512, 520, 528, 536, 544, 552, 560, 568, 576, 584, 592, 600, 608, 616, 624, 626, 628, 630, 632, 634, 636, 638, 646, 654, 663, 779, 781, 785 and 787, or any other VH sequence within a full length polypeptide chain sequence disclosed herein. In some aspects, the sequence has a mutation at N62, for example, N62Q or N62Q at, for example, SEQ ID NO:279. In some preferred aspects, one or more of VH1, VH2, VH3 and VH4 have at least 90% identity with the corresponding amino acid sequences specified above, optionally with a mutation at N62 as specified above. As used herein, “at least 90% identity” includes at least 91%, 92%, 93%, 94%, 95%, 96%, 97%, 98%, 99% and 100% identity to the recited reference sequence. In some particularly preferred aspects, one or more of VH1, VH2, VH3 and VH4 comprise or consist of the corresponding amino acid sequences specified above, optionally with a mutation at N62 as specified above. In some preferred aspects, VH1, VH2, VH3 and VH4 have at least 90% identity with the corresponding amino acid sequences specified above, optionally with a mutation at N62 as specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3 and VH4 comprise or consist of the corresponding amino acid sequences specified above, optionally with a mutation at N62 as specified above.

In some aspects, one or more of VL1, VL2, VL3 and VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:38, 46, 54, 62, 70, 166, 174, 182, 259, 267, 275, 283, 508, 509, 511, 519, 527, 535, 543, 551, 559, 567, 575, 583, 588, 596, 604, 612, 620, 625, 627, 629, 631, 633, 635, 637, 642, 650, 659 and 667, or any other VL sequence within a full length polypeptide chain sequence disclosed herein. In some preferred aspects, one or more of VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, one or more of VL1, VL2, VL3 and VL4 comprise or consist of the corresponding amino acid sequences specified above. In some preferred aspects, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VL1, VL2, VL3 and VL4 comprise or consist of the corresponding amino acid sequences specified above.

In some aspects, one or more of VH1, VH2, VH3 and VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:42, 50, 58, 66, 74, 170, 178, 186, 263, 271, 279, 287, 510, 512, 520, 528, 536, 544, 552, 560, 568, 576, 584, 592, 600, 608, 616, 624, 626, 628, 630, 632, 634, 636, 638, 646, 654, 663, 779, 781, 785 and 787; and one or more of VL1, VL2, VL3 and VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:38, 46, 54, 62, 70, 166, 174, 182, 259, 267, 275, 283, 508, 509, 511, 516, 524, 532, 540, 548, 556, 564, 572, 580, 588, 596, 604, 612, 620, 625, 627, 629, 631, 633, 635, 637, 642, 650, 659 and 667, or any other VH or VL sequence within a full length polypeptide chain sequence disclosed herein. In some aspects, the sequence has a mutation at N62, for example, N62Q or N62Q at, for example, SEQ ID NO:279. In some preferred aspects, one or more of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, one or more of VH1, VH2, VH3, VH4, VL1, VL2, VL3 and VL4 comprise or consist of the corresponding amino acid sequences specified above. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above.

In some aspects, one or more of VH1, VH2, VH3 and VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:513; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:514; and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:515; and one or more of VL1, VL2, VL3 and VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:517; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:518 (having the sequence of DAS); and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:519. In some aspects, CDR1, CDR2 and CDR3 of one or more of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, CDR1, CDR2 and CDR3 of one or more of VH1, VH2, VH3, VH4, VL1, VL2, VL3 and VL4 comprise or consist of the corresponding amino acid sequences specified above. In some preferred aspects, CDR1, CDR2 and CDR3 of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, VH4, VL1, VL2, VL3 and VL4 comprise or consist of the corresponding amino acid sequences specified above.

In some aspects, one or more of VH1, VH2, VH3 and VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:512; and one or more of VL1, VL2, VL3 and VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:516. In some aspects, one or more of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, one or more of VH1, VH2, VH3, VH4, VL1, VL2, VL3 and VL4 comprise or consist of the corresponding amino acid sequences specified above. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some preferred aspects, VH1, VH2, VH3, VH4, VL1, VL2, VL3 and VL4 comprise or consist of the corresponding amino acid sequences specified above.

In some aspects, one or more of VH1, VH2, VH3 and VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:521; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:522; and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:523; and one or more of VL1, VL2, VL3 and VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:525; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:526 (having the sequence of DVS); and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:527. In some aspects, CDR1, CDR2 and CDR3 of one or more of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, CDR1, CDR2 and CDR3 of one or more of VH1, VH2, VH3, VH4, VL1, VL2, VL3 and VL4 comprise or consist of the corresponding amino acid sequences specified above. In some preferred aspects, CDR1, CDR2 and CDR3 of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, VH4, VL1, VL2, VL3 and VL4 comprise or consist of the corresponding amino acid sequences specified above.

In some aspects, one or more of VH1, VH2, VH3 and VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:520; and one or more of VL1, VL2, VL3 and VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:524. In some aspects, one or more of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, one or more of VH1, VH2, VH3, VH4, VL1, VL2, VL3 and VL4 comprise or consist of the corresponding amino acid sequences specified above. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some preferred aspects, VH1, VH2, VH3, VH4, VL1, VL2, VL3 and VL4 comprise or consist of the corresponding amino acid sequences specified above.

In some aspects, one or more of VH1, VH2, VH3 and VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:529; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:530; and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:531; and one or more of VL1, VL2, VL3 and VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:533; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:534 (having the sequence of EDS); and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:535. In some aspects, CDR1, CDR2 and CDR3 of one or more of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, CDR1, CDR2 and CDR3 of one or more of VH1, VH2, VH3, VH4, VL1, VL2, VL3 and VL4 comprise or consist of the corresponding amino acid sequences specified above. In some preferred aspects, CDR1, CDR2 and CDR3 of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, VH4, VL1, VL2, VL3 and VL4 comprise or consist of the corresponding amino acid sequences specified above.

In some aspects, one or more of VH1, VH2, VH3 and VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:528; and one or more of VL1, VL2, VL3 and VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:532. In some aspects, one or more of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, one or more of VH1, VH2, VH3, VH4, VL1, VL2, VL3 and VL4 comprise or consist of the corresponding amino acid sequences specified above. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some preferred aspects, VH1, VH2, VH3, VH4, VL1, VL2, VL3 and VL4 comprise or consist of the corresponding amino acid sequences specified above.

In some aspects, one or more of VH1, VH2, VH3 and VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:537; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:538; and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:539; and one or more of VL1, VL2, VL3 and VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:541; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:542 (having the sequence of KDS); and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:543. In some aspects, CDR1, CDR2 and CDR3 of one or more of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, CDR1, CDR2 and CDR3 of one or more of VH1, VH2, VH3, VH4, VL1, VL2, VL3 and VL4 comprise or consist of the corresponding amino acid sequences specified above. In some preferred aspects, CDR1, CDR2 and CDR3 of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, VH4, VL1, VL2, VL3 and VL4 comprise or consist of the corresponding amino acid sequences specified above.

In some aspects, one or more of VH1, VH2, VH3 and VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:536; and one or more of VL1, VL2, VL3 and VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:540. In some aspects, one or more of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, one or more of VH1, VH2, VH3, VH4, VL1, VL2, VL3 and VL4 comprise or consist of the corresponding amino acid sequences specified above. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above.

In some aspects, one or more of VH1, VH2, VH3 and VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:545; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:546; and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:547; and one or more of VL1, VL2, VL3 and VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:549; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:550 (having the sequence of DAS); and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:551. In some aspects, CDR1, CDR2 and CDR3 of one or more of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, CDR1, CDR2 and CDR3 of one or more of VH1, VH2, VH3, VH4, VL1, VL2, VL3 and VL4 comprise or consist of the corresponding amino acid sequences specified above. In some preferred aspects, CDR1, CDR2 and CDR3 of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, VH4, VL1, VL2, VL3 and VL4 comprise or consist of the corresponding amino acid sequences specified above.

In some aspects, one or more of VH1, VH2, VH3 and VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:544; and one or more of VL1, VL2, VL3 and VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:548. In some aspects, one or more of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, one or more of VH1, VH2, VH3, VH4, VL1, VL2, VL3 and VL4 comprise or consist of the corresponding amino acid sequences specified above. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some preferred aspects, VH1, VH2, VH3, VH4, VL1, VL2, VL3 and VL4 comprise or consist of the corresponding amino acid sequences specified above.

In some aspects, one or more of VH1, VH2, VH3 and VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:624; and one or more of VL1, VL2, VL3 and VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:625. In some aspects, one or more of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, one or more of VH1, VH2, VH3, VH4, VL1, VL2, VL3 and VL4 comprise or consist of the corresponding amino acid sequences specified above. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some preferred aspects, VH1, VH2, VH3, VH4, VL1, VL2, VL3 and VL4 comprise or consist of the corresponding amino acid sequences specified above.

In some aspects, one or more of VH1, VH2, VH3 and VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:626; and one or more of VL1, VL2, VL3 and VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:627. In some aspects, one or more of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, one or more of VH1, VH2, VH3, VH4, VL1, VL2, VL3 and VL4 comprise or consist of the corresponding amino acid sequences specified above. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above.

In some aspects, one or more of VH1, VH2, VH3 and VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:628; and one or more of VL1, VL2, VL3 and VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:629. In some aspects, one or more of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, one or more of VH1, VH2, VH3, VH4, VL1, VL2, VL3 and VL4 comprise or consist of the corresponding amino acid sequences specified above. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above.

In some aspects, one or more of VH1, VH2, VH3 and VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:630; and one or more of VL1, VL2, VL3 and VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:631. In some aspects, one or more of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, one or more of VH1, VH2, VH3, VH4, VL1, VL2, VL3 and VL4 comprise or consist of the corresponding amino acid sequences specified above. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above.

In some aspects, one or more of VH1, VH2, VH3 and VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:632; and one or more of VL1, VL2, VL3 and VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:633. In some aspects, one or more of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, one or more of VH1, VH2, VH3, VH4, VL1, VL2, VL3 and VL4 comprise or consist of the corresponding amino acid sequences specified above. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above.

In some aspects, one or more of VH1, VH2, VH3 and VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:634; and one or more of VL1, VL2, VL3 and VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:635. In some aspects, one or more of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, one or more of VH1, VH2, VH3, VH4, VL1, VL2, VL3 and VL4 comprise or consist of the corresponding amino acid sequences specified above. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above.

In some aspects, one or more of VH1, VH2, VH3 and VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:636; and one or more of VL1, VL2, VL3 and VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:637. In some aspects, one or more of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, one or more of VH1, VH2, VH3, VH4, VL1, VL2, VL3 and VL4 comprise or consist of the corresponding amino acid sequences specified above. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some preferred aspects, VH1, VH2, VH3, VH4, VL1, VL2, VL3 and VL4 comprise or consist of the corresponding amino acid sequences specified above.

In some aspects, one or more of VH1, VH2, VH3 and VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:638; and one or more of VL1, VL2, VL3 and VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:642. In some aspects, one or more of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, one or more of VH1, VH2, VH3, VH4, VL1, VL2, VL3 and VL4 comprise or consist of the corresponding amino acid sequences specified above. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above.

In some aspects, one or more of VH1, VH2, VH3 and VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:646; and one or more of VL1, VL2, VL3 and VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:650. In some aspects, one or more of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, one or more of VH1, VH2, VH3, VH4, VL1, VL2, VL3 and VL4 comprise or consist of the corresponding amino acid sequences specified above. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above.

In some aspects, one or more of VH1, VH2, VH3 and VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:654; and one or more of VL1, VL2, VL3 and VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:659. In some aspects, one or more of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, one or more of VH1, VH2, VH3, VH4, VL1, VL2, VL3 and VL4 comprise or consist of the corresponding amino acid sequences specified above. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some preferred aspects, VH1, VH2, VH3, VH4, VL1, VL2, VL3 and VL4 comprise or consist of the corresponding amino acid sequences specified above.

In some aspects, one or more of VH1, VH2, VH3 and VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:663; and one or more of VL1, VL2, VL3 and VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:667. In some aspects, one or more of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, one or more of VH1, VH2, VH3, VH4, VL1, VL2, VL3 and VL4 comprise or consist of the corresponding amino acid sequences specified above. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above.

In some aspects, one or more of VH1, VH2, VH3 and VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:639; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:640; and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:641; and one or more of VL1, VL2, VL3 and VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:643; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:644 (having the sequence of GAS); and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:645. In some aspects, CDR1, CDR2 and CDR3 of one or more of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, CDR1, CDR2 and CDR3 of one or more of VH1, VH2, VH3, VH4, VL1, VL2, VL3 and VL4 comprise or consist of the corresponding amino acid sequences specified above. In some preferred aspects, CDR1, CDR2 and CDR3 of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, VH4, VL1, VL2, VL3 and VL4 comprise or consist of the corresponding amino acid sequences specified above.

In some aspects, one or more of VH1, VH2, VH3 and VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:647; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:648; and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:649; and one or more of VL1, VL2, VL3 and VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:651; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:652 (having the sequence of SAS); and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:653. In some aspects, CDR1, CDR2 and CDR3 of one or more of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, CDR1, CDR2 and CDR3 of one or more of VH1, VH2, VH3, VH4, VL1, VL2, VL3 and VL4 comprise or consist of the corresponding amino acid sequences specified above. In some preferred aspects, CDR1, CDR2 and CDR3 of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, VH4, VL1, VL2, VL3 and VL4 comprise or consist of the corresponding amino acid sequences specified above.

In some aspects, one or more of VH1, VH2, VH3 and VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:656; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:657; and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:658; and one or more of VL1, VL2, VL3 and VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:660; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:661 (having the sequence of GAS); and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:662. In some aspects, CDR1, CDR2 and CDR3 of one or more of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, CDR1, CDR2 and CDR3 of one or more of VH1, VH2, VH3, VH4, VL1, VL2, VL3 and VL4 comprise or consist of the corresponding amino acid sequences specified above. In some preferred aspects, CDR1, CDR2 and CDR3 of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, VH4, VL1, VL2, VL3 and VL4 comprise or consist of the corresponding amino acid sequences specified above.

In some aspects, one or more of VH1, VH2, VH3 and VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:664; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:665; and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:666; and one or more of VL1, VL2, VL3 and VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:668; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:669 (having the sequence of DAS); and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:670. In some aspects, CDR1, CDR2 and CDR3 of one or more of VH1, VH2, VH3, VH4, VL1, VL2, VL3 and VL4 have at least 90% identity with the corresponding amino acid sequences specified above. In some aspects, CDR1, CDR2 and CDR3 of one or more of VH1, VH2, VH3, VH4, VL1, VL2, VL3 and VL4 comprise or consist of the corresponding amino acid sequences specified above. As used herein, “at least 90% identity” includes at least 91%, 92%, 93%, 94%, 95%, 96%, 97%, 98%, 99% and 100% identity to the recited reference sequence. In some preferred aspects, CDR1, CDR2 and CDR3 of VH1, VH2, VH3, VH4, VL1, VL2, VL3 and VL4 have at least 90% identity with the corresponding amino acid sequences specified above. In some particularly preferred aspects, CDR1, CDR2 and CDR3 of VH1, VH2, VH3, VH4, VL1, VL2, VL3 and VL4 comprise or consist of the corresponding amino acid sequences specified above.

In some aspects, VH1, VH2, VH3 and VH4 each comprise the same CDR1, CDR2 and CDR3 regions. In some aspects, VL1, VL2, VL3 and VL4 each comprise the same CDR1, CDR2 and CDR3 regions. In some aspects, VH1, VH2, VH3 and VH4 each comprise the same CDR1, CDR2 and CDR3 regions, and VL1, VL2, VL3 and VL4 each comprise the same CDR1, CDR2 and CDR3 regions.

In some aspects, VH1, VH2, VH3 and VH4 each comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666. In some preferred aspects, CDR1, CDR2 and CDR3 of VH1, VH2, VH3 and VH4 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, and VH4 each comprise or consist of a corresponding amino acid sequence specified above.

In some aspects, VL1, VL2, VL3 and VL4 each comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:39, 47, 55, 63, 71, 167, 175, 183, 260, 268, 276, 284, 517, 525, 533, 541, 549, 557, 565, 573, 581, 589, 597, 605, 613, 621, 643, 651, 660 and 668; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:40, 48, 56, 64, 72, 168, 176, 184, 261, 269, 277 (having the sequence of LGS), 285, 518 (having the sequence of DAS), 526 (having the sequence of DVS), 534 (having the sequence of EDS), 542 (having the sequence of KDS), 550 (having the sequence of DAS), 558 (having the sequence of AAS), 566 (having the sequence of GAS), 574 (having the sequence of DDS), 582 (having the sequence of KDS), 590 (having the sequence of SAS), 598 (having the sequence of SAS), 606 (having the sequence of GAS), 614 (having the sequence of GAS), 622 (having the sequence of GAS), 644 (having the sequence of GAS), 652 (having the sequence of SAS), 661 (having the sequence of GAS) and 669 (having the sequence of DAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:41, 49, 57, 65, 73, 169, 177, 185, 262, 270, 278, 286, 519, 527, 535, 543, 551, 559, 567, 575, 583, 591, 599, 607, 615, 623, 645, 653, 662 and 670. In some preferred aspects, CDR1, CDR2 and CDR3 of VL1, VL2, VL3 and VL4 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VL1, VL2, VL3 and VL4 each comprise or consist of a corresponding amino acid sequence specified above.

In some aspects, VH1, VH2, VH3 and VH4 each comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666; and VL1, VL2, VL3 and VL4 each comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:39, 47, 55, 63, 71, 167, 175, 183, 260, 268, 276, 284, 517, 525, 533, 541, 549, 557, 565, 573, 581, 589, 597, 605, 613, 621, 643, 651, 660 and 668; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:40, 48, 56, 64, 72, 168, 176, 184, 261, 269, 277 (having the sequence of LGS), 285, 518 (having the sequence of DAS), 526 (having the sequence of DVS), 534 (having the sequence of EDS), 542 (having the sequence of KDS), 550 (having the sequence of DAS), 558 (having the sequence of AAS), 566 (having the sequence of GAS), 574 (having the sequence of DDS), 582 (having the sequence of KDS), 590 (having the sequence of SAS), 598 (having the sequence of SAS), 606 (having the sequence of GAS), 614 (having the sequence of GAS), 622 (having the sequence of GAS), 644 (having the sequence of GAS), 652 (having the sequence of SAS), 661 (having the sequence of GAS) and 669 (having the sequence of DAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:41, 49, 57, 65, 73, 169, 177, 185, 262, 270, 278, 286, 519, 527, 535, 543, 551, 559, 567, 575, 583, 591, 599, 607, 615, 623, 645, 653, 662 and 670. In some preferred aspects, CDR1, CDR2 and CDR3 of VH1, VH2, VH3, VH4, VL1, VL2, VL3 and VL4 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, VH4, VL1, VL2, VL3 and VL4 each comprise or consist of a corresponding amino acid sequence specified above.

In some aspects, VH1, VH2, VH3 and VH4 each comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:43; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:44; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:45; and VL1, VL2, VL3 and VL4 each comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:39; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:40 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:41. In some preferred aspects, CDR1, CDR2 and CDR3 of VH1, VH2, VH3, VH4, VL1, VL2, VL3 and VL4 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, VH4, VL1, VL2, VL3 and VL4 each comprise or consist of a corresponding amino acid sequence specified above.

In some aspects, VH1, VH2, VH3 and VH4 each comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:51; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:52; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:53; and VL1, VL2, VL3 and VL4 each comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:47; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:48 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:49. In some preferred aspects, CDR1, CDR2 and CDR3 of VH1, VH2, VH3, VH4, VL1, VL2, VL3 and VL4 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, VH4, VL1, VL2, VL3 and VL4 each comprise or consist of a corresponding amino acid sequence specified above.

In some aspects, VH1, VH2, VH3 and VH4 each comprise the same heavy chain variable region. In some aspects, VL1, VL2, VL3 and VL4 each comprise the same light chain variable region. In some aspects, VH1, VH2, VH3 and VH4 each comprise the same heavy chain variable region, and VL1, VL2, VL3 and VL4 each comprise the same light chain variable region.

In some aspects, VH1, VH2, VH3 and VH4 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:42, 50, 58, 66, 74, 170, 178, 186, 263, 271, 279, 287, 510, 512, 520, 528, 536, 544, 552, 560, 568, 576, 584, 592, 600, 608, 616, 624, 626, 628, 630, 632, 634, 636, 638, 646, 654, 663, 779, 781, 785 and 787. In some aspects, VL1, VL2, VL3 and VL4 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:38, 46, 54, 62, 70, 166, 174, 182, 259, 267, 275, 283, 508, 509, 511, 516, 524, 532, 540, 548, 556, 564, 572, 580, 588, 596, 604, 612, 620, 625, 627, 629, 631, 633, 635, 637, 642, 650, 659 and 667. In some aspects, VH1, VH2, VH3 and VH4 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:42, 50, 58, 66, 74, 170, 178, 186, 263, 271, 279, 287, 510, 512, 520, 528, 536, 544, 552, 560, 568, 576, 584, 592, 600, 608, 616, 624, 626, 628, 630, 632, 634, 636, 638, 646, 654, 663, 779, 781, 785 and 787, and VL1, VL2, VL3 and VL4 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:38, 46, 54, 62, 70, 166, 174, 182, 259, 267, 275, 283, 508, 509, 511, 516, 524, 532, 540, 548, 556, 564, 572, 580, 588, 596, 604, 612, 620, 625, 627, 629, 631, 633, 635, 637, 642, 650, 659 and 667. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 each have at least 90% identity with a corresponding amino acid sequence specified above, optionally with a mutation at N62 as described above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 each comprise or consist of a corresponding amino acid sequence specified above, optionally with a mutation at N62 as described above.

In some aspects, VH1, VH2, VH3 and VH4 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:42. In some aspects, VL1, VL2, VL3 and VL4 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:38. In some aspects, VH1, VH2, VH3 and VH4 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:42, and VL1, VL2, VL3 and VL4 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:38. In some preferred aspects, VH1, VH2, VH3, VH4, VL1, VL2, VL3 and VL4 each have at least 90% identity with the corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 each comprise or consist of the corresponding amino acid sequence specified above.

In some aspects, VH1, VH2, VH3 and VH4 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:50. In some aspects, VL1, VL2, VL3 and VL4 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:46. In some aspects, VH1, VH2, VH3 and VH4 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:50, and VL1, VL2, VL3 and VL4 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:46. In some preferred aspects, VH1, VH2, VH3, VH4, VL1, VL2, VL3 and VL4 each have at least 90% identity with the corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 each comprise or consist of the corresponding amino acid sequence specified above.

In some aspects, VH1 comprises the same CDR1, CDR2 and CDR3 regions as VH3. In some aspects, VL1 comprises the same CDR1, CDR2 and CDR3 regions as VL3. In some aspects, VH1 comprises the same CDR1, CDR2 and CDR3 regions as VH3, and VL1 comprises the same CDR1, CDR2 and CDR3 regions as VL3.

In some aspects, VH2 comprises the same CDR1, CDR2 and CDR3 regions as VH4. In some aspects, VL2 comprises the same CDR1, CDR2 and CDR3 regions as VL4. In some aspects, VH2 comprises the same CDR1, CDR2 and CDR3 regions as VH4, and VL2 comprises the same CDR1, CDR2 and CDR3 regions as VL4.

In some aspects, VH1 comprises the same CDR1, CDR2 and CDR3 regions as VH3, VL1 comprises the same CDR1, CDR2 and CDR3 regions as VL3, VH2 comprises the same CDR1, CDR2 and CDR3 regions as VH4, and VL2 comprises the same CDR1, CDR2 and CDR3 regions as VL4.

In some aspects, VH1 CDR1 and VH3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664. In some aspects, VH1 CDR2 and VH3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665. In some aspects, VH1 CDR3 and VH3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666. In some preferred aspects, CDR1, CDR2 and CDR3 of VH1 and VH3 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1 and VH3 each comprise or consist of a corresponding amino acid sequence specified above.

In some aspects, VH1 CDR1 and VH3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664; VH1 CDR2 and VH3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; and VH1 CDR3 and VH3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666. In some preferred aspects, CDR1, CDR2 and CDR3 of VH1 and VH3 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1 and VH3 each comprise or consist of a corresponding amino acid sequence specified above.

In some aspects, VL1 CDR1 and VL3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:39, 47, 55, 63, 71, 167, 175, 183, 260, 268, 276, 284, 517, 525, 533, 541, 549, 557, 565, 573, 581, 589, 597, 605, 613, 621, 643, 651, 660 and 668. In some aspects, VL1 CDR2 and VL3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:40, 48, 56, 64, 72, 168, 176, 184, 261, 269, 277 (having the sequence of LGS), 285, 518 (having the sequence of DAS), 526 (having the sequence of DVS), 534 (having the sequence of EDS), 542 (having the sequence of KDS), 550 (having the sequence of DAS), 558 (having the sequence of AAS), 566 (having the sequence of GAS), 574 (having the sequence of DDS), 582 (having the sequence of KDS), 590 (having the sequence of SAS), 598 (having the sequence of SAS), 606 (having the sequence of GAS), 614 (having the sequence of GAS), 622 (having the sequence of GAS), 644 (having the sequence of GAS), 652 (having the sequence of SAS), 661 (having the sequence of GAS) and 669 (having the sequence of DAS). In some aspects, VL1 CDR3 and VL3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:41, 49, 57, 65, 73, 169, 177, 185, 262, 270, 278, 286, 519, 527, 535, 543, 551, 559, 567, 575, 583, 591, 599, 607, 615, 623, 645, 653, 662 and 670. In some preferred aspects, CDR1, CDR2 and CDR3 of VL1 and VL3 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VL1 and VL3 each comprise or consist of a corresponding amino acid sequence specified above.

In some aspects, VL1 CDR1 and VL3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:39, 47, 55, 63, 71, 167, 175, 183, 260, 268, 276, 284, 517, 525, 533, 541, 549, 557, 565, 573, 581, 589, 597, 605, 613, 621, 643, 651, 660 and 668; VL1 CDR2 and VL3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:40, 48, 56, 64, 72, 168, 176, a184, 261, 269, 277 (having the sequence of LGS), 285, 518 (having the sequence of DAS), 526 (having the sequence of DVS), 534 (having the sequence of EDS), 542 (having the sequence of KDS), 550 (having the sequence of DAS), 558 (having the sequence of AAS), 566 (having the sequence of GAS), 574 (having the sequence of DDS), 582 (having the sequence of KDS), 590 (having the sequence of SAS), 598 (having the sequence of SAS), 606 (having the sequence of GAS), 614 (having the sequence of GAS), 622 (having the sequence of GAS), 644 (having the sequence of GAS), 652 (having the sequence of SAS), 661 (having the sequence of GAS) and 669 (having the sequence of DAS); and VL1 CDR3 and VL3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:41, 49, 57, 65, 73, 169, 177, 185, 262, 270, 278, 286, 519, 527, 535, 543, 551, 559, 567, 575, 583, 591, 599, 607, 615, 623, 645, 653, 662 and 670. In some preferred aspects, CDR1, CDR2 and CDR3 of VL1 and VL3 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VL1 and VL3 each comprise or consist of a corresponding amino acid sequence specified above.

In some aspects, VH1 CDR1 and VH3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664; VH1 CDR2 and VH3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; VH1 CDR3 and VH3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666; VL1 CDR1 and VL3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:39, 47, 55, 63, 71, 167, 175, 183, 260, 268, 276, 284, 517, 525, 533, 541, 549, 557, 565, 573, 581, 589, 597, 605, 613, 621, 643, 651, 660 and 668; VL1 CDR2 and VL3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:40, 48, 56, 64, 72, 168, 176, 184, 261, 269, 277 (having the sequence of LGS), 285, 518 (having the sequence of DAS), 526 (having the sequence of DVS), 534 (having the sequence of EDS), 542 (having the sequence of KDS), 550 (having the sequence of DAS), 558 (having the sequence of AAS), 566 (having the sequence of GAS), 574 (having the sequence of DDS), 582 (having the sequence of KDS), 590 (having the sequence of SAS), 598 (having the sequence of SAS), 606 (having the sequence of GAS), 614 (having the sequence of GAS), 622 (having the sequence of GAS), 644 (having the sequence of GAS), 652 (having the sequence of SAS), 661 (having the sequence of GAS) and 669 (having the sequence of DAS); and VL1 CDR3 and VL3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:41, 49, 57, 65, 73, 169, 177, 185, 262, 270, 278, 286, 519, 527, 535, 543, 551, 559, 567, 575, 583, 591, 599, 607, 615, 623, 645, 653, 662 and 670. In some preferred aspects, CDR1, CDR2 and CDR3 of VH1, VH3, VL1 and VL3 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH3, VL1 and VL3 each comprise or consist of a corresponding amino acid sequence specified above.

In some aspects, VH2 CDR1 and VH4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664. In some aspects, VH2 CDR2 and VH4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665. In some aspects, VH2 CDR3 and VH4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666. In some preferred aspects, CDR1, CDR2 and CDR3 of VH2 and VH4 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH2 and VH4 each comprise or consist of a corresponding amino acid sequence specified above.

In some aspects, VH2 CDR1 and VH4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664; VH2 CDR2 and VH4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; and VH2 CDR3 and VH4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666. In some preferred aspects, CDR1, CDR2 and CDR3 of VH2 and VH4 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH2 and VH4 each comprise or consist of a corresponding amino acid sequence specified above.

In some aspects, VL2 CDR1 and VL4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:39, 47, 55, 63, 71, 167, 175, 183, 260, 268, 276, 284, 517, 525, 533, 541, 549, 557, 565, 573, 581, 589, 597, 605, 613, 621, 643, 651, 660 and 668. In some aspects, VL2 CDR2 and VL4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:40, 48, 56, 64, 72, 168, 176, 184, 261, 269, 277 (having the sequence of LGS), 285, 518 (having the sequence of DAS), 526 (having the sequence of DVS), 534 (having the sequence of EDS), 542 (having the sequence of KDS), 550 (having the sequence of DAS), 558 (having the sequence of AAS), 566 (having the sequence of GAS), 574 (having the sequence of DDS), 582 (having the sequence of KDS), 590 (having the sequence of SAS), 598 (having the sequence of SAS), 606 (having the sequence of GAS), 614 (having the sequence of GAS), 622 (having the sequence of GAS), 644 (having the sequence of GAS), 652 (having the sequence of SAS), 661 (having the sequence of GAS) and 669 (having the sequence of DAS). In some aspects, VL2 CDR3 and VL4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:41, 49, 57, 65, 73, 169, 177, 185, 262, 270, 278, 286, 519, 527, 535, 543, 551, 559, 567, 575, 583, 591, 599, 607, 615, 623, 645, 653, 662 and 670. In some preferred aspects, CDR1, CDR2 and CDR3 of VL2 and VL4 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VL2 and VL4 each comprise or consist of a corresponding amino acid sequence specified above.

In some aspects, VL2 CDR1 and VL4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:39, 47, 55, 63, 71, 167, 175, 183, 260, 268, 276, 284, 517, 525, 533, 541, 549, 557, 565, 573, 581, 589, 597, 605, 613, 621, 643, 651, 660 and 668; VL2 CDR2 and VL4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:40, 48, 56, 64, 72, 168, 176, 184, 261, 269, 277 (having the sequence of LGS), 285, 518 (having the sequence of DAS), 526 (having the sequence of DVS), 534 (having the sequence of EDS), 542 (having the sequence of KDS), 550 (having the sequence of DAS), 558 (having the sequence of AAS), 566 (having the sequence of GAS), 574 (having the sequence of DDS), 582 (having the sequence of KDS), 590 (having the sequence of SAS), 598 (having the sequence of SAS), 606 (having the sequence of GAS), 614 (having the sequence of GAS), 622 (having the sequence of GAS), 644 (having the sequence of GAS), 652 (having the sequence of SAS), 661 (having the sequence of GAS) and 669 (having the sequence of DAS); and VL2 CDR3 and VL4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:41, 49, 57, 65, 73, 169, 177, 185, 262, 270, 278, 286, 519, 527, 535, 543, 551, 559, 567, 575, 583, 591, 599, 607, 615, 623, 645, 653, 662 and 670. In some preferred aspects, CDR1, CDR2 and CDR3 of VL2 and VL4 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VL2 and VL4 each comprise or consist of a corresponding amino acid sequence specified above.

In some aspects, VH2 CDR1 and VH4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664; VH2 CDR2 and VH4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; VH2 CDR3 and VH4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666; VL2 CDR1 and VL4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:39, 47, 55, 63, 71, 167, 175, 183, 260, 268, 276, 284, 517, 525, 533, 541, 549, 557, 565, 573, 581, 589, 597, 605, 613, 621, 643, 651, 660 and 668; VL2 CDR2 and VL4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:40, 48, 56, 64, 72, 168, 176, 184, 261, 269, 277 (having the sequence of LGS), 285, 518 (having the sequence of DAS), 526 (having the sequence of DVS), 534 (having the sequence of EDS), 542 (having the sequence of KDS), 550 (having the sequence of DAS), 558 (having the sequence of AAS), 566 (having the sequence of GAS), 574 (having the sequence of DDS), 582 (having the sequence of KDS), 590 (having the sequence of SAS), 598 (having the sequence of SAS), 606 (having the sequence of GAS), 614 (having the sequence of GAS), 622 (having the sequence of GAS), 644 (having the sequence of GAS), 652 (having the sequence of SAS), 661 (having the sequence of GAS) and 669 (having the sequence of DAS); and VL2 CDR3 and VL4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:41, 49, 57, 65, 73, 169, 177, 185, 262, 270, 278, 286, 519, 527, 535, 543, 551, 559, 567, 575, 583, 591, 599, 607, 615, 623, 645, 653, 662 and 670. In some preferred aspects, CDR1, CDR2 and CDR3 of VH2, VH4, VL2 and VL4 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH2, VH4, VL2 and VL4 each comprise or consist of a corresponding amino acid sequence specified above.

In some aspects, VH1 CDR1 and VH3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; VH1 CDR2 and VH3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; VH1 CDR3 and VH3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; VL1 CDR1 and VL3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; VL1 CDR2 and VL3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); VL1 CDR3 and VL3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; VH2 CDR1 and VH4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:43; VH2 CDR2 and VH4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:44; VH2 CDR3 and VH4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:45; VL2 CDR1 and VL4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:39; VL2 CDR2 and VL4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:40 (having the sequence of GAS); and VL2 CDR3 and VL4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:41. In some preferred aspects, CDR1, CDR2 and CDR3 of VH1, VH2, VH3, VH4, VL1, VL2, VL3 and VL4 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, VH4, VL1, VL2, VL3 and VL4 each comprise or consist of a corresponding amino acid sequence specified above.

In some aspects, VH1 CDR1 and VH3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:43; VH1 CDR2 and VH3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:44; VH1 CDR3 and VH3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:45; VL1 CDR1 and VL3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:39; VL1 CDR2 and VL3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:40 (having the sequence of GAS); VL1 CDR3 and VL3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:41; VH2 CDR1 and VH4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; VH2 CDR2 and VH4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; VH2 CDR3 and VH4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; VL2 CDR1 and VL4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; VL2 CDR2 and VL4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and VL2 CDR3 and VL4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57. In some preferred aspects, CDR1, CDR2 and CDR3 of VH1, VH2, VH3, VH4, VL1, VL2, VL3 and VL4 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, VH4, VL1, VL2, VL3 and VL4 each comprise or consist of a corresponding amino acid sequence specified above.

In some aspects, VH1 CDR1 and VH3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:67; VH1 CDR2 and VH3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:68; VH1 CDR3 and VH3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:69; VL1 CDR1 and VL3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:63; VL1 CDR2 and VL3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:64 (having the sequence of DAS); VL1 CDR3 and VL3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:65; VH2 CDR1 and VH4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:43; VH2 CDR2 and VH4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:44; VH2 CDR3 and VH4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:45; VL2 CDR1 and VL4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:39; VL2 CDR2 and VL4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:40 (having the sequence of GAS); and VL2 CDR3 and VL4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:41. In some preferred aspects, CDR1, CDR2 and CDR3 of VH1, VH2, VH3, VH4, VL1, VL2, VL3 and VL4 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, VH4, VL1, VL2, VL3 and VL4 each comprise or consist of a corresponding amino acid sequence specified above.

In some aspects, VH1 CDR1 and VH3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:43; VH1 CDR2 and VH3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:44; VH1 CDR3 and VH3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:45; VL1 CDR1 and VL3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:39; VL1 CDR2 and VL3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:40 (having the sequence of GAS); VL1 CDR3 and VL3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:41; VH2 CDR1 and VH4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:67; VH2 CDR2 and VH4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:68; VH2 CDR3 and VH4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:69; VL2 CDR1 and VL4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:63; VL2 CDR2 and VL4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:64 (having the sequence of DAS); and VL2 CDR3 and VL4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:65. In some preferred aspects, CDR1, CDR2 and CDR3 of VH1, VH2, VH3, VH4, VL1, VL2, VL3 and VL4 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, VH4, VL1, VL2, VL3 and VL4 each comprise or consist of a corresponding amino acid sequence specified above.

In some aspects, VH1 CDR1 and VH3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:51; VH1 CDR2 and VH3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:52; VH1 CDR3 and VH3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:53; VL1 CDR1 and VL3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:47; VL1 CDR2 and VL3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:48 (having the sequence of GAS); VL1 CDR3 and VL3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:49; VH2 CDR1 and VH4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:67; VH2 CDR2 and VH4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:68; VH2 CDR3 and VH4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:69; VL2 CDR1 and VL4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:63; VL2 CDR2 and VL4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:64 (having the sequence of DAS); and VL2 CDR3 and VL4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:65. In some preferred aspects, CDR1, CDR2 and CDR3 of VH1, VH2, VH3, VH4, VL1, VL2, VL3 and VL4 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, VH4, VL1, VL2, VL3 and VL4 each comprise or consist of a corresponding amino acid sequence specified above.

In some aspects, VH1 CDR1 and VH3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:67; VH1 CDR2 and VH3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:68; VH1 CDR3 and VH3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:69; VL1 CDR1 and VL3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:63; VL1 CDR2 and VL3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:64 (having the sequence of DAS); VL1 CDR3 and VL3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:65; VH2 CDR1 and VH4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:51; VH2 CDR2 and VH4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:52; VH2 CDR3 and VH4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:53; VL2 CDR1 and VL4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:47; VL2 CDR2 and VL4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:48 (having the sequence of GAS); and VL2 CDR3 and VL4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:49. In some preferred aspects, CDR1, CDR2 and CDR3 of VH1, VH2, VH3, VH4, VL1, VL2, VL3 and VL4 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, VH4, VL1, VL2, VL3 and VL4 each comprise or consist of a corresponding amino acid sequence specified above.

In some aspects, VH1 CDR1 and VH3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:51; VH1 CDR2 and VH3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:52; VH1 CDR3 and VH3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:53; VL1 CDR1 and VL3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:47; VL1 CDR2 and VL3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:48 (having the sequence of GAS); VL1 CDR3 and VL3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:49; VH2 CDR1 and VH4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; VH2 CDR2 and VH4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; VH2 CDR3 and VH4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; VL2 CDR1 and VL4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; VL2 CDR2 and VL4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and VL2 CDR3 and VL4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57. In some preferred aspects, CDR1, CDR2 and CDR3 of VH1, VH2, VH3, VH4, VL1, VL2, VL3 and VL4 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, VH4, VL1, VL2, VL3 and VL4 each comprise or consist of a corresponding amino acid sequence specified above.

In some aspects, VH1 CDR1 and VH3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; VH1 CDR2 and VH3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; VH1 CDR3 and VH3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; VL1 CDR1 and VL3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; VL1 CDR2 and VL3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); VL1 CDR3 and VL3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; VH2 CDR1 and VH4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:51; VH2 CDR2 and VH4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:52; VH2 CDR3 and VH4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:53; VL2 CDR1 and VL4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:47; VL2 CDR2 and VL4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:48 (having the sequence of GAS); and VL2 CDR3 and VL4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:49. In some preferred aspects, CDR1, CDR2 and CDR3 of VH1, VH2, VH3, VH4, VL1, VL2, VL3 and VL4 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, VH4, VL1, VL2, VL3 and VL4 each comprise or consist of a corresponding amino acid sequence specified above.

In some aspects, VH1 CDR1 and VH3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:75; VH1 CDR2 and VH3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:76; VH1 CDR3 and VH3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:77; VL1 CDR1 and VL3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:71; VL1 CDR2 and VL3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:72 (having the sequence of SAS); VL1 CDR3 and VL3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:73; VH2 CDR1 and VH4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:67; VH2 CDR2 and VH4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:68; VH2 CDR3 and VH4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:69; VL2 CDR1 and VL4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:63; VL2 CDR2 and VL4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:64 (having the sequence of DAS); and VL2 CDR3 and VL4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:65. In some preferred aspects, CDR1, CDR2 and CDR3 of VH1, VH2, VH3, VH4, VL1, VL2, VL3 and VL4 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, VH4, VL1, VL2, VL3 and VL4 each comprise or consist of a corresponding amino acid sequence specified above.

In some aspects, VH1 CDR1 and VH3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:67; VH1 CDR2 and VH3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:68; VH1 CDR3 and VH3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:69; VL1 CDR1 and VL3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:63; VL1 CDR2 and VL3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:64 (having the sequence of DAS); VL1 CDR3 and VL3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:65; VH2 CDR1 and VH4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:75; VH2 CDR2 and VH4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:76; VH2 CDR3 and VH4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:77; VL2 CDR1 and VL4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:71; VL2 CDR2 and VL4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:72 (having the sequence of SAS); and VL2 CDR3 and VL4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:73. In some preferred aspects, CDR1, CDR2 and CDR3 of VH1, VH2, VH3, VH4, VL1, VL2, VL3 and VL4 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, VH4, VL1, VL2, VL3 and VL4 each comprise or consist of a corresponding amino acid sequence specified above.

In some aspects, VH1 comprises the same heavy chain variable region as VH3 (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:42, 50, 58, 66, 74, 170, 178, 186, 263, 271, 279, 287, 510, 512, 520, 528, 536, 544, 552, 560, 568, 576, 584, 592, 600, 608, 616, 624, 626, 628, 630, 632, 634, 636, 638, 646, 654, 663, 779, 781, 785 and 787). In some aspects, VL1 comprises the same light chain variable region as VL3 (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:38, 46, 54, 62, 70, 166, 174, 182, 259, 267, 275, 283, 508, 509, 511, 516, 524, 532, 540, 548, 556, 564, 572, 580, 588, 596, 604, 612, 620, 625, 627, 629, 631, 633, 635, 637, 642, 650, 659 and 667). In some aspects, VH1 comprises the same heavy chain variable region as VH3 (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:42, 50, 58, 66, 74, 170, 178, 186, 263, 271, 279, 287, 510, 512, 520, 528, 536, 544, 552, 560, 568, 576, 584, 592, 600, 608, 616, 624, 626, 628, 630, 632, 634, 636, 638, 646, 654, 663, 779, 781, 785 and 787), and VL1 comprises the same light chain variable region as VL3 (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:38, 46, 54, 62, 70, 166, 174, 182, 259, 267, 275, 283, 508, 509, 511, 516, 524, 532, 540, 548, 556, 564, 572, 580, 588, 596, 604, 612, 620, 625, 627, 629, 631, 633, 635, 637, 642, 650, 659 and 667). In some aspects, the sequence has a mutation at N62, for example, N62Q or N62Q at, for example, SEQ ID NO:279. In some preferred aspects, VH1, VH3, VL1 and VL3 each have at least 90% identity with a corresponding amino acid sequence specified above, optionally with a mutation at N62 as described above. As used herein, “at least 90% identity” includes at least 91%, 92%, 93%, 94%, 95%, 96%, 97%, 98%, 99% and 100% identity to the recited reference sequence. In some particularly preferred aspects, VH1, VH3, VL1 and VL3 each comprise or consist of a corresponding amino acid sequence specified above, optionally with a mutation at N62 as described above.

In some aspects, VH2 comprises the same heavy chain variable region as VH4 (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:42, 50, 58, 66, 74, 170, 178, 186, 263, 271, 279, 287, 510, 512, 520, 528, 536, 544, 552, 560, 568, 576, 584, 592, 600, 608, 616, 624, 626, 628, 630, 632, 634, 636, 638, 646, 654, 663, 779, 781, 785 and 787). In some aspects, VL2 comprises the same light chain variable region as VL4 (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:38, 46, 54, 62, 70, 166, 174, 182, 259, 267, 275, 283, 508, 509, 511, 516, 524, 532, 540, 548, 556, 564, 572, 580, 588, 596, 604, 612, 620, 625, 627, 629, 631, 633, 635, 637, 642, 650, 659 and 667). In some aspects, VH2 comprises the same heavy chain variable region as VH4 (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:42, 50, 58, 66, 74, 170, 178, 186, 263, 271, 279, 287, 510, 512, 520, 528, 536, 544, 552, 560, 568, 576, 584, 592, 600, 608, 616, 624, 626, 628, 630, 632, 634, 636, 638, 646, 654, 663, 779, 781, 785 and 787), and VL2 comprises the same light chain variable region as VL4 (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:38, 46, 54, 62, 70, 166, 174, 182, 259, 267, 275, 283, 508, 509, 511, 516, 524, 532, 540, 548, 556, 564, 572, 580, 588, 596, 604, 612, 620, 625, 627, 629, 631, 633, 635, 637, 642, 650, 659 and 667). In some aspects, the sequence has a mutation at N62, for example, N62Q or N62Q at, for example, SEQ ID NO:279. In some preferred aspects, VH2, VH4, VL2 and VL4 each have at least 90% identity with a corresponding amino acid sequence specified above, optionally with a mutation at N62 as described above. As used herein, “at least 90% identity” includes at least 91%, 92%, 93%, 94%, 95%, 96%, 97%, 98%, 99% and 100% identity to the recited reference sequence. In some particularly preferred aspects, VH2, VH4, VL2 and VL4 each comprise or consist of a corresponding amino acid sequence specified above, optionally with a mutation at N62 as described above.

In some aspects, VH1 comprises the same heavy chain variable region as VH3 (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:42, 50, 58, 66, 74, 170, 178, 186, 263, 271, 279, 287, 510, 512, 520, 528, 536, 544, 552, 560, 568, 576, 584, 592, 600, 608, 616, 624, 626, 628, 630, 632, 634, 636, 638, 646, 654, 663, 779, 781, 785 and 787); VL1 comprises the same light chain variable region as VL3 (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:38, 46, 54, 62, 70, 166, 174, 182, 259, 267, 275, 283, 508, 509, 511, 516, 524, 532, 540, 548, 556, 564, 572, 580, 588, 596, 604, 612, 620, 625, 627, 629, 631, 633, 635, 637, 642, 650, 659 and 667); VH2 comprises the same heavy chain variable region as VH4 (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:42, 50, 58, 66, 74, 170, 178, 186, 263, 271, 279, 287, 510, 512, 520, 528, 536, 544, 552, 560, 568, 576, 584, 592, 600, 608, 616, 624, 626, 628, 630, 632, 634, 636, 638, 646, 654, 663, 779, 781, 785 and 787); and VL2 comprises the same light chain variable region as VL4 (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:38, 46, 54, 62, 70, 166, 174, 182, 259, 267, 275, 283, 508, 509, 511, 516, 524, 532, 540, 548, 556, 564, 572, 580, 588, 596, 604, 612, 620, 625, 627, 629, 631, 633, 635, 637, 642, 650, 659 and 667). In some aspects, the sequence has a mutation at N62, for example, N62Q or N62Q at, for example, SEQ ID NO:279. In some preferred aspects, VH1, VH2, VH3, VH4, VL1, VL2, VL3 and VL4 each have at least 90% identity with a corresponding amino acid sequence specified above, optionally with a mutation at N62 as described above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 each comprise or consist of a corresponding amino acid sequence specified above, optionally with a mutation at N62 as described above.

In some aspects, VH1 and VH3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:58; VL1 and VL3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:54; VH2 and VH4 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:42; and VL2 and VL4 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:38. In some preferred aspects, VH1, VH2, VH3, VH4, VL1, VL2, VL3 and VL4 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 each comprise or consist of a corresponding amino acid sequence specified above.

In some aspects, VH1 and VH3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:42; VL1 and VL3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:38; VH2 and VH4 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:58; and VL2 and VL4 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:54. In some preferred aspects, VH1, VH2, VH3, VH4, VL1, VL2, VL3 and VL4 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 each comprise or consist of a corresponding amino acid sequence specified above.

In some aspects, VH1 and VH3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:66; VL1 and VL3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:62; VH2 and VH4 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:42; and VL2 and VL4 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:38. In some preferred aspects, VH1, VH2, VH3, VH4, VL1, VL2, VL3 and VL4 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 each comprise or consist of a corresponding amino acid sequence specified above.

In some aspects, VH1 and VH3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:42; VL1 and VL3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:38; VH2 and VH4 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:66; and VL2 and VL4 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:62. In some preferred aspects, VH1, VH2, VH3, VH4, VL1, VL2, VL3 and VL4 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 each comprise or consist of a corresponding amino acid sequence specified above.

In some aspects, VH1 and VH3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:50; VL1 and VL3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:46; VH2 and VH4 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:66; and VL2 and VL4 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:62. In some preferred aspects, VH1, VH2, VH3, VH4, VL1, VL2, VL3 and VL4 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 each comprise or consist of a corresponding amino acid sequence specified above.

In some aspects, VH1 and VH3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:66; VL1 and VL3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:62; VH2 and VH4 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:50; and VL2 and VL4 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:46. In some preferred aspects, VH1, VH2, VH3, VH4, VL1, VL2, VL3 and VL4 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 each comprise or consist of a corresponding amino acid sequence specified above.

In some aspects, VH1 and VH3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:50; VL1 and VL3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:46; VH2 and VH4 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:58; and VL2 and VL4 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:54. In some preferred aspects, VH1, VH2, VH3, VH4, VL1, VL2, VL3 and VL4 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 each comprise or consist of a corresponding amino acid sequence specified above.

In some aspects, VH1 and VH3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:58; VL1 and VL3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:54; VH2 and VH4 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:50; and VL2 and VL4 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:46. In some preferred aspects, VH1, VH2, VH3, VH4, VL1, VL2, VL3 and VL4 each have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 each comprise or consist of a corresponding amino acid sequence specified above.

In some aspects, two of VH1, VH2, VH3 and VH4 comprise the same CDR1, CDR2 and CDR3 regions (e.g., a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666). In some preferred aspects, CDR1, CDR2 and CDR3 of each of VH1, VH2, VH3 and VH4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3 and VH4 comprise or consist of the corresponding amino acid sequences specified above.

In some aspects, two of VL1, VL2, VL3 and VL4 comprise the same CDR1, CDR2 and CDR3 regions (e.g., a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:39, 47, 55, 63, 71, 167, 175, 183, 260, 268, 276, 284, 589, 597, 605, 613, 621, 643, 651, 660 and 668; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:40, 48, 56, 64, 72, 168, 176, 184, 261, 269, 277 (having the sequence of LGS), 285, 518 (having the sequence of DAS), 526 (having the sequence of DVS), 534 (having the sequence of EDS), 542 (having the sequence of KDS), 550 (having the sequence of DAS), 558 (having the sequence of AAS), 566 (having the sequence of GAS), 574 (having the sequence of DDS), 582 (having the sequence of KDS), 590 (having the sequence of SAS), 598 (having the sequence of SAS), 606 (having the sequence of GAS), 614 (having the sequence of GAS), 622 (having the sequence of GAS), 644 (having the sequence of GAS), 652 (having the sequence of SAS), 661 (having the sequence of GAS) and 669 (having the sequence of DAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:41, 49, 57, 65, 73, 169, 177, 185, 262, 270, 278, 286, 519, 527, 535, 543, 551, 559, 567, 575, 583, 591, 599, 607, 615, 623, 645, 653, 662 and 670). In some preferred aspects, CDR1, CDR2 and CDR3 of each of VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VL1, VL2, VL3 and VL4 comprise or consist of the corresponding amino acid sequences specified above.

In some aspects, two of VH1, VH2, VH3 and VH4 comprise the same CDR1, CDR2 and CDR3 regions (e.g., a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666); and two of VL1, VL2, VL3 and VL4 comprise the same CDR1, CDR2 and CDR3 regions (e.g., a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:39, 47, 55, 63, 71, 167, 175, 183, 260, 268, 276, 284, 517, 525, 533, 541, 549, 557, 565, 573, 581, 589, 597, 605, 613, 621, 643, 651, 660 and 668; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:40, 48, 56, 64, 72, 168, 176, 184, 261, 269, 277 (having the sequence of LGS), 285, 518 (having the sequence of DAS), 526 (having the sequence of DVS), 534 (having the sequence of EDS), 542 (having the sequence of KDS), 550 (having the sequence of DAS), 558 (having the sequence of AAS), 566 (having the sequence of GAS), 574 (having the sequence of DDS), 582 (having the sequence of KDS), 590 (having the sequence of SAS), 598 (having the sequence of SAS), 606 (having the sequence of GAS), 614 (having the sequence of GAS), 622 (having the sequence of GAS), 644 (having the sequence of GAS), 652 (having the sequence of SAS), 661 (having the sequence of GAS) and 669 (having the sequence of DAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:41, 49, 57, 65, 73, 169, 177, 185, 262, 270, 278, 286, 519, 527, 535, 543, 551, 559, 567, 575, 583, 591, 599, 607, 615, 623, 645, 653, 662 and 670). In some preferred aspects, CDR1, CDR2 and CDR3 of two of VH1, VH2, VH3, VH4; and CDR1, CDR2 and CDR3 of two of VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of two of VH1, VH2, VH3, VH4; and CDR1, CDR2 and CDR3 of two of VL1, VL2, VL3 and VL4 comprise or consist of the corresponding amino acid sequences specified above. In some preferred aspects, CDR1, CDR2 and CDR3 of three of VH1, VH2, VH3, VH4; and CDR1, CDR2 and CDR3 of three of VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of three of VH1, VH2, VH3, VH4; and CDR1, CDR2 and CDR3 of three of VL1, VL2, VL3 and VL4 comprise or consist of the corresponding amino acid sequences specified above. In some preferred aspects, CDR1, CDR2 and CDR3 of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, VH4, VL1, VL2, VL3 and VL4 comprise or consist of the corresponding amino acid sequences specified above.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:58; VH2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:50; VH3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:66; VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:74; VL1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:54; VL2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:46; VL3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:62; and VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:38. In some aspects, the linker between VL1 and VL2 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL2 and VH2 has at least 80%, at least 85%, at least 90%, at least 91%, at least 92%, at least 93%, at least 94%, at least 95%, at least 96%, at least 97%, at least 98%, at least 99% or 100% identity to SEQ ID NO:27. In some aspects, the linker between VH2 and VH1 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL3 and VL4 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL4 and VH4 has at least 80%, at least 85%, at least 90%, at least 91%, at least 92%, at least 93%, at least 94%, at least 95%, at least 96%, at least 97%, at least 98%, at least 99% or 100% identity to SEQ ID NO:27. In some aspects, the linker between VH4 and VH3 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above.

In some aspects, VH1 comprises an amino acid sequence of SEQ ID NO:58; VH2 comprises an amino acid sequence of SEQ ID NO:50; VH3 comprises an amino acid sequence of SEQ ID NO:66; VH4 comprises an amino acid sequence of SEQ ID NO:74; VL1 comprises an amino acid sequence of SEQ ID NO:54; VL2 comprises an amino acid sequence of SEQ ID NO:46; VL3 comprises an amino acid sequence of SEQ ID NO:62; and VL4 comprises an amino acid of SEQ ID NO:38. In some aspects, the linker between VL1 and VL2 is SEQ ID NO:6. In some aspects, the linker between VL2 and VH2 is SEQ ID NO:27. In some aspects, the linker between VH2 and VH1 is SEQ ID NO:6. In some aspects, the linker between VL3 and VL4 is SEQ ID NO:6. In some aspects, the linker between VL4 and VH4 is SEQ ID NO:27. In some aspects, the linker between VH4 and VH3 is SEQ ID NO:6.

In some aspects, VH2 comprises the same CDR1, CDR2 and CDR3 regions as VH4. In some aspects, VL2 comprises the same CDR1, CDR2 and CDR3 regions as VL4. In some aspects, VH2 comprises the same CDR1, CDR2 and CDR3 regions as VH4, and VL2 comprises the same CDR1, CDR2 and CDR3 regions as VL4. In some aspects, VH1 comprises different CDR1, CDR2 and CDR3 regions than VH3 and/or VL1 comprises different CDR1, CDR2 and CDR3 regions than VL3.

In some aspects, VH2 CDR1 and VH4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:43; VH2 CDR2 and VH4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:44; VH2 CDR3 and VH4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:45; VL2 CDR1 and VL2 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:39; VL2 CDR2 and VL4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:40 (having the sequence of GAS); VL2 CDR3 and VL4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:41. In some aspects, VH1 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:67; VH1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:68; VH1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:69; VL1 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:63; VL1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:64 (having the sequence of DAS); and VL1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:65. In some aspects, VH3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; VH3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; VH3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; VL3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; VL3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and VL3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57. In some preferred aspects, CDR1, CDR2 and CDR3 of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, VH4, VL1, VL2, VL3 and VL4 comprise or consist of the corresponding amino acid sequences specified above.

In some aspects, VH2 CDR1 and VH4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:51; VH2 CDR2 and VH4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:52; VH2 CDR3 and VH4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:53; VL2 CDR1 and VL4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:47; VL2 CDR2 and VL4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:48 (having the sequence of GAS); and VL2 CDR3 and VL4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:49. In some aspects, VH1 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; VH1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; VH1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; VL1 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; VL1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and VL1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57. In some aspects, VH3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:67; VH3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:68; VH3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:69; VL3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:63; VL3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:64 (having the sequence of DAS); and VL3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:65. In some preferred aspects, CDR1, CDR2 and CDR3 of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, VH4, VL1, VL2, VL3 and VL4 comprise or consist of the corresponding amino acid sequences specified above.

In some aspects, VH2 comprises the same CDR1, CDR2 and CDR3 regions as VH3. In some aspects, VL2 comprises the same CDR1, CDR2 and CDR3 regions as VL3. In some aspects, VH2 comprises the same CDR1, CDR2 and CDR3 regions as VH3, and VL2 comprises the same CDR1, CDR2 and CDR3 regions as VL3. In some aspects, VH1 comprises different CDR1, CDR2 and CDR3 regions than VH4 and/or VL1 comprises different CDR1, CDR2 and CDR3 regions than VL4.

In some aspects, VH2 CDR1 and VH3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:43; VH2 CDR2 and VH3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:44; VH2 CDR3 and VH3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:45; VL2 CDR1 and VL3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:39; VL2 CDR2 and VL3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:40 (having the sequence of GAS); and VL2 CDR3 and VL3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:41. In some aspects, VH1 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:67; VH1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:68; VH1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:69; VL1 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:63; VL1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:64 (having the sequence of DAS); and VL1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:65. In some aspects, VH4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; VH4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; VH4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; VL4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; VL4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and VL4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57. In some preferred aspects, CDR1, CDR2 and CDR3 of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, VH4, VL1, VL2, VL3 and VL4 comprise or consist of the corresponding amino acid sequences specified above.

In some aspects, VH2 CDR1 and VH3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:51; VH2 CDR2 and VH3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:52; VH2 CDR3 and VH3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:53; VL2 CDR1 and VL3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:47; VL2 CDR2 and VL3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:48 (having the sequence of GAS); and VL2 CDR3 and VL3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:49. In some aspects, VH1 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; VH1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; VH1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; VL1 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; VL1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and VL1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57. In some aspects, VH4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:67; VH4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:68; VH4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:69; VL4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:63; VL4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:64 (having the sequence of DAS); and VL4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:65. In some preferred aspects, CDR1, CDR2 and CDR3 of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, VH4, VL1, VL2, VL3 and VL4 comprise or consist of the corresponding amino acid sequences specified above.

In some aspects, VH1 comprises the same CDR1, CDR2 and CDR3 regions as VH4. In some aspects, VL1 comprises the same CDR1, CDR2 and CDR3 regions as VL4. In some aspects, VH1 comprises the same CDR1, CDR2 and CDR3 regions as VH4, and VL1 comprises the same CDR1, CDR2 and CDR3 regions as VL4. In some aspects, VH2 comprises different CDR1, CDR2 and CDR3 regions than VH3 and/or VL2 comprises different CDR1, CDR2 and CDR3 regions than VL3.

In some aspects, VH1 CDR1 and VH4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:43; VH1 CDR2 and VH4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:44; VH1 CDR3 and VH4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:45; VL1 CDR1 and VL4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:39; VL1 CDR2 and VL4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:40 (having the sequence of GAS); and VL1 CDR3 and VL4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:41. In some aspects, VH2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:67; VH2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:68; VH2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:69; VL2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:63; VL2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:64 (having the sequence of DAS); and VL2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:65. In some aspects, VH3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; VH3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; VH3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; VL3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; VL3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and VL3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57. In some preferred aspects, CDR1, CDR2 and CDR3 of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, VH4, VL1, VL2, VL3 and VL4 comprise or consist of the corresponding amino acid sequences specified above.

In some aspects, VH1 CDR1 and VH4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:51; VH1 CDR2 and VH4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:52; VH1 CDR3 and VH4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:53; VL1 CDR1 and VL4 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:47; VL1 CDR2 and VL4 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:48 (having the sequence of GAS); and VL1 CDR3 and VL4 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:49. In some aspects, VH2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; VH2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; VH2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; VL2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; VL2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and VL2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57. In some aspects, VH3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:67; VH3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:68; VH3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:69; VL3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:63; VL3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:64 (having the sequence of DAS); and VL3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:65. In some preferred aspects, CDR1, CDR2 and CDR3 of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, VH4, VL1, VL2, VL3 and VL4 comprise or consist of the corresponding amino acid sequences specified above.

In some aspects, VH1 comprises the same CDR1, CDR2 and CDR3 regions as VH3. In some aspects, VL1 comprises the same CDR1, CDR2 and CDR3 regions as VL3. In some aspects, VH1 comprises the same CDR1, CDR2 and CDR3 regions as VH3, and VL1 comprises the same CDR1, CDR2 and CDR3 regions as VL3. In some aspects, VH2 comprises different CDR1, CDR2 and CDR3 regions than VH4 and/or VL2 comprises different CDR1, CDR2 and CDR3 regions than VL4.

In some aspects, VH1 CDR1 and VH3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:43; VH1 CDR2 and VH3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:44; VH1 CDR3 and VH3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:45; VL1 CDR1 and VL3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:39; VL1 CDR2 and VL3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:40 (having the sequence of GAS); and VL1 CDR3 and VL3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:41. In some aspects, VH2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:67; VH2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:68; VH2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:68; VL2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:63; VL2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:64 (having the sequence of DAS); and VL2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:65. In some aspects, VH4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; VH4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; VH4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; VL4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; VL4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and VL4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57. In some preferred aspects, CDR1, CDR2 and CDR3 of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, VH4, VL1, VL2, VL3 and VL4 comprise or consist of the corresponding amino acid sequences specified above.

In some aspects, VH1 CDR1 and VH3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:51; VH1 CDR2 and VH3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:52; VH1 CDR3 and VH3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:53; VL1 CDR1 and VL3 CDR1 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:47; VL1 CDR2 and VL3 CDR2 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:48 (having the sequence of GAS); and VL1 CDR3 and VL3 CDR3 each comprise the same amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:49. In some aspects, VH2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; VH2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; VH2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; VL2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; VL2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and VL2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57. In some aspects, VH4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:67; VH4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:68; VH4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:69; VL4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:63; VL4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:64 (having the sequence of DAS); and VL4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:65. In some preferred aspects, CDR1, CDR2 and CDR3 of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of VH1, VH2, VH3, VH4, VL1, VL2, VL3 and VL4 comprise or consist of the corresponding amino acid sequences specified above.

In some aspects, VH2 comprises the same heavy chain variable region as VH4. In some aspects, VL2 comprises the same light chain variable region as VL4. In some aspects, VH2 comprises the same heavy chain variable region as VH4, and VL2 comprises the same light chain variable region as VL4. In some aspects, VH2 and VH4 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:42, 50, 58, 58, 66, 74, 170, 178, 186, 263, 271, 279, 287, 510, 512, 520, 528, 536, 544, 552, 560, 568, 576, 584, 592, 600, 608, 616, 624, 626, 628, 630, 632, 634, 636, 638, 646, 654, 663, 779, 781, 785 and 787, and/or VL2 and VL4 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:38, 46, 54, 62, 70, 166, 174, 182, 259, 267, 275, 283, 508, 509, 511, 516, 524, 532, 540, 548, 556, 564, 572, 580, 588, 596, 604, 612, 620, 625, 627, 629, 631, 633, 635, 637, 642, 650, 659 and 667. In some aspects, VH1 and VH3 comprise different heavy chain variable regions than VH2 and VH4 (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:42, 50, 58, 58, 66, 74, 170, 178, 186, 263, 271, 279, 287, 510, 512, 520, 528, 536, 544, 552, 560, 568, 576, 584, 592, 600, 608, 616, 624, 626, 628, 630, 632, 634, 636, 638, 646, 654, 663, 779, 781, 785 and 787), and/or VL1 and VL3 comprise different light chain variable regions than VL2 and VL4 (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:38, 46, 54, 62, 70, 166, 174, 182, 259, 267, 275, 283, 508, 509, 511, 516, 524, 532, 540, 548, 556, 564, 572, 580, 588, 596, 604, 612, 620, 625, 627, 629, 631, 633, 635, 637, 642, 650, 659 and 667). In some aspects, VH1 comprises a different heavy chain variable region than VH3, and/or VL1 comprises a different light chain variable region than VL3. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with a mutation at N62 as described above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above, optionally with a mutation at N62 as described above.

In some aspects, VH2 and VH4 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:42. In some aspects, VL2 and VL4 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:38. In some aspects, VH2 and VH4 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:42, and VL2 and VL4 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:38. In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:66, and/or VL1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:62. In some aspects, VH3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:58, and/or VL3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:54. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above.

In some aspects, VH2 and VH4 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:50. In some aspects, VL2 and VL4 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:46. In some aspects, VH2 and VH4 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:50, and VL2 and VL4 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:46. In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:58, and/or VL1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:54. In some aspects, VH3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:66, and/or VL3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:62. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above.

In some aspects, VH2 comprises the same heavy chain variable region as VH3. In some aspects, VL2 comprises the same light chain variable region as VL3. In some aspects, VH2 comprises the same heavy chain variable region as VH3, and VL2 comprises the same light chain variable region as VL3. In some aspects, VH2 and VH3 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:42, 50, 58, 58, 66, 74, 170, 178, 186, 263, 271, 279, 287, 510, 512, 520, 528, 536, 544, 552, 560, 568, 576, 584, 592, 600, 608, 616, 624, 626, 628, 630, 632, 634, 636, 638, 646, 654, 663, 779, 781, 785 and 787, and/or VL2 and VL3 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:38, 46, 54, 62, 70, 166, 174, 182, 259, 267, 275, 283, 508, 509, 511, 516, 524, 532, 540, 548, 556, 564, 572, 580, 588, 596, 604, 612, 620, 625, 627, 629, 631, 633, 635, 637, 642, 650, 659 and 667. In some aspects, VH1 and VH4 comprise different heavy chain variable regions than VH2 and VH3 (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:42, 50, 58, 58, 66, 74, 170, 178, 186, 263, 271, 279, 287, 510, 512, 520, 528, 536, 544, 552, 560, 568, 576, 584, 592, 600, 608, 616, 624, 626, 628, 630, 632, 634, 636, 638, 646, 654, 663, 779, 781, 785 and 787), and/or VL1 and VL4 comprise different light chain variable regions than VL2 and VL3 (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:38, 46, 54, 62, 70, 166, 174, 182, 259, 267, 275, 283, 508, 509, 511, 516, 524, 532, 540, 548, 556, 564, 572, 580, 588, 596, 604, 612, 620, 625, 627, 629, 631, 633, 635, 637, 642, 650, 659 and 667). In some aspects, VH1 comprises a different heavy chain variable region than VH4, and/or VL1 comprises a different light chain variable region than VL4. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with a mutation at N62 as described above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above, optionally with a mutation at N62 as described above.

In some aspects, VH2 and VH3 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:42. In some aspects, VL2 and VL3 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:38. In some aspects, VH2 and VH3 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:42, and VL2 and VL3 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:38. In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:66, and/or VL1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:62. In some aspects, VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:58, and/or VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:54. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above.

In some aspects, VH2 and VH3 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:50. In some aspects, VL2 and VL3 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:46. In some aspects, VH2 and VH3 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:50, and VL2 and VL3 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:46. In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:58, and/or VL1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:54. In some aspects, VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:66, and/or VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:62. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above.

In some aspects, VH1 comprises the same heavy chain variable region as VH4. In some aspects, VL1 comprises the same light chain variable region as VL4. In some aspects, VH1 comprises the same heavy chain variable region as VH4, and VL1 comprises the same light chain variable region as VL4. In some aspects, VH1 and VH4 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:42, 50, 58, 58, 66, 74, 170, 178, 186, 263, 271, 279, 287, 510, 512, 520, 528, 536, 544, 552, 560, 568, 576, 584, 592, 600, 608, 616, 624, 626, 628, 630, 632, 634, 636, 638, 646, 654, 663, 779, 781, 785 and 787, and/or VL1 and VL4 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:38, 46, 54, 62, 70, 166, 174, 182, 259, 267, 275, 283, 508, 509, 511, 516, 524, 532, 540, 548, 556, 564, 572, 580, 588, 596, 604, 612, 620, 625, 627, 629, 631, 633, 635, 637, 642, 650, 659 and 667. In some aspects, VH2 and VH3 comprise different heavy chain variable regions than VH1 and VH4 (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:42, 50, 58, 58, 66, 74, 170, 178, 186, 263, 271, 279, 287, 510, 512, 520, 528, 536, 544, 552, 560, 568, 576, 584, 592, 600, 608, 616, 624, 626, 628, 630, 632, 634, 636, 638, 646, 654, 663, 779, 781, 785 and 787, and/or VL2 and VL3 comprise different light chain variable regions than VL1 and VL4 (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:38, 46, 54, 62, 70, 166, 174, 182, 259, 267, 275, 283, 508, 509, 511, 516, 524, 532, 540, 548, 556, 564, 572, 580, 588, 596, 604, 612, 620, 625, 627, 629, 631, 633, 635, 637, 642, 650, 659 and 667). In some aspects, VH2 comprises a different heavy chain variable region than VH3, and/or VL2 comprises a different light chain variable region than VL3. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with a mutation at N62 as described above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above, optionally with a mutation at N62 as described above.

In some aspects, VH1 and VH4 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:42. In some aspects, VL1 and VL4 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:38. In some aspects, VH1 and VH4 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:42, and VL1 and VL4 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:38. In some aspects, VH2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:66, and/or VL2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:62. In some aspects, VH3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:58, and/or VL3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:54. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above.

In some aspects, VH1 and VH4 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:50. In some aspects, VL1 and VL4 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:46. In some aspects, VH1 and VH4 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:50, and VL1 and VL4 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:46. In some aspects, VH2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:58, and/or VL2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:54. In some aspects, VH3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:66, and/or VL3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:62. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above.

In some aspects, VH1 comprises the same heavy chain variable region as VH3. In some aspects, VL1 comprises the same light chain variable region as VL3. In some aspects, VH1 comprises the same heavy chain variable region as VH3, and VL1 comprises the same light chain variable region as VL3. In some aspects, VH1 and VH3 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:42, 50, 58, 58, 66, 74, 170, 178, 186, 263, 271, 279, 287, 510, 512, 520, 528, 536, 544, 552, 560, 568, 576, 584, 592, 600, 608, 616, 624, 626, 628, 630, 632, 634, 636, 638, 646, 654, 663, 779, 781, 785 and 787, and/or VL1 and VL3 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:38, 46, 54, 62, 70, 166, 174, 182, 259, 267, 275, 283, 508, 509, 511, 516, 524, 532, 540, 548, 556, 564, 572, 580, 588, 596, 604, 612, 620, 625, 627, 629, 631, 633, 635, 637, 642, 650, 659 and 667. In some aspects, VH2 and VH4 comprise different heavy chain variable regions than VH1 and VH3 (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:42, 50, 58, 58, 66, 74, 170, 178, 186, 263, 271, 279, 287, 510, 512, 520, 528, 536, 544, 552, 560, 568, 576, 584, 592, 600, 608, 616, 624, 626, 628, 630, 632, 634, 636, 638, 646, 654, 663, 779, 781, 785 and 787), and/or VL2 and VL4 comprise different light chain variable regions than VL1 and VL3 (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:38, 46, 54, 62, 70, 166, 174, 182, 259, 267, 275, 283, 508, 509, 511, 516, 524, 532, 540, 548, 556, 564, 572, 580, 588, 596, 604, 612, 620, 625, 627, 629, 631, 633, 635, 637, 642, 650, 659 and 667). In some aspects, VH2 comprises a different heavy chain variable region than VH4, and/or VL2 comprises a different light chain variable region than VL4. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with a mutation at N62 as described above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above, optionally with a mutation at N62 as described above.

In some aspects, VH1 and VH3 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:42. In some aspects, VL1 and VL3 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:38. In some aspects, VH1 and VH3 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:42, and VL1 and VL3 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:38. In some aspects, VH2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:66, and/or VL2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:62. In some aspects, VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:58, and/or VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:54. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above.

In some aspects, VH1 and VH3 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:50. In some aspects, VL1 and VL3 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:46. In some aspects, VH1 and VH3 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:50, and VL1 and VL3 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:46. In some aspects, VH2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:58, and/or VL2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:54. In some aspects, VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:66, and/or VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:62. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above.

In some aspects, each of VH1, VH2, VH3 and VH4 comprise different CDR1, CDR2 and/or CDR3 regions (e.g., a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666). In some preferred aspects, CDR1, CDR2 and CDR3 of each of VH1, VH2, VH3 and VH have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3 and VH4 comprise or consist of the corresponding amino acid sequences specified above.

In some aspects, each of VL1, VL2, VL3 and VL4 comprise different CDR1, CDR2 and/or CDR3 regions (e.g., a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:39, 47, 55, 63, 71, 167, 175, 183, 260, 268, 276, 284, 517, 525, 533, 541, 549, 557, 565, 573, 581, 589, 597, 605, 613, 621, 643, 651, 660 and 668; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:40, 48, 56, 64, 72, 168, 176, 184, 261, 269, 277 (having the sequence of LGS), 285, 518 (having the sequence of DAS), 526 (having the sequence of DVS), 534 (having the sequence of EDS), 542 (having the sequence of KDS), 550 (having the sequence of DAS), 558 (having the sequence of AAS), 566 (having the sequence of GAS), 574 (having the sequence of DDS), 582 (having the sequence of KDS), 590 (having the sequence of SAS), 598 (having the sequence of SAS), 606 (having the sequence of GAS), 614 (having the sequence of GAS), 622 (having the sequence of GAS, 644 (having the sequence of GAS), 652 (having the sequence of SAS), 661 (having the sequence of GAS) and 669 (having the sequence of DAS); and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:41, 49, 57, 65, 73, 169, 177, 185, 262, 270, 278, 286, 519, 527, 535, 543, 551, 559, 567, 575, 583, 591, 599, 607, 615, 623, 645, 653, 662 and 670). In some preferred aspects, CDR1, CDR2 and CDR3 of each of VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VL1, VL2, VL3 and VL4 comprise or consist of the corresponding amino acid sequences specified above.

In some aspects, each of VH1, VH2, VH3 and VH4 comprise different CDR1, CDR2 and/or CDR3 regions (e.g., a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666); and each of VL1, VL2, VL3 and VL4 comprise different CDR1, CDR2 and/or CDR3 regions (e.g., a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:39, 47, 55, 63, 71, 167, 175, 183, 260, 268, 276, 284, 517, 525, 533, 541, 549, 557, 565, 573, 581, 589, 597, 605, 613, 621, 643, 651, 660 and 668; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:40, 48, 56, 64, 72, 168, 176, 184, 261, 269, 277 (having the sequence of LGS), 285, 518 (having the sequence of DAS), 526 (having the sequence of DVS), 534 (having the sequence of EDS), 542 (having the sequence of KDS), 550 (having the sequence of DAS), 558 (having the sequence of AAS), 566 (having the sequence of GAS), 574 (having the sequence of DDS), 582 (having the sequence of KDS), 590 (having the sequence of SAS), 598 (having the sequence of SAS), 606 (having the sequence of GAS), 614 (having the sequence of GAS), 622 (having the sequence of GAS), 644 (having the sequence of GAS), 652 (having the sequence of SAS), 661 (having the sequence of GAS) and 669 (having the sequence of DAS); and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:41, 49, 57, 65, 73, 169, 177, 185, 262, 270, 278, 286, 519, 527, 535, 543, 551, 559, 567, 575, 583, 591, 599, 607, 615, 623, 645, 653, 662 and 670). In some preferred aspects, CDR1, CDR2 and CDR3 of each of VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VH4, VL1, VL2, VL3 and VL4 comprise or consist of the corresponding amino acid sequences specified above.

In some aspects, the VH1 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; the VH1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; the VH1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; the VH2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:51; the VH2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:52; the VH2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:53; the VH3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:67; the VH3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:68; the VH3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:69; the VH4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:75; the VH4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:76; the VH4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:77; the VL1 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; the VL1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); the VL1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; the VL2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:47; the VL2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:48 (having the sequence of GAS); the VL2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:49; the VL3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:63; the VL3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:64 (having the sequence of DAS); the VL3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:65; the VL4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:39; the VL4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:40 (having the sequence of GAS); and the VL4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:41. In some aspects, the linker between VL1 and VL2 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL2 and VH2 has at least 80%, at least 85%, at least 90%, at least 91%, at least 92%, at least 93%, at least 94%, at least 95%, at least 96%, at least 97%, at least 98%, at least 99% or 100% identity to SEQ ID NO:27. In some aspects, the linker between VH2 and VH1 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL3 and VL4 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL4 and VH4 has at least 80%, at least 85%, at least 90%, at least 91%, at least 92%, at least 93%, at least 94%, at least 95%, at least 96%, at least 97%, at least 98%, at least 99% or 100% identity to SEQ ID NO:27. In some aspects, the linker between VH4 and VH3 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with one or more linker sequence as specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with one or more linker sequence as specified above.

In some aspects, the VH1 CDR1 comprises an amino acid sequence of SEQ ID NO:59; the VH1 CDR2 comprises an amino acid sequence of SEQ ID NO:60; the VH1 CDR3 comprises an amino acid sequence of SEQ ID NO:61; the VH2 CDR1 comprises an amino acid sequence of SEQ ID NO:51; the VH2 CDR2 comprises an amino acid sequence of SEQ ID NO:52; the VH2 CDR3 comprises an amino acid sequence of SEQ ID NO:53; the VH3 CDR1 comprises an amino acid sequence of SEQ ID NO:67; the VH3 CDR2 comprises an amino acid sequence of SEQ ID NO:68; the VH3 CDR3 comprises an amino acid sequence of SEQ ID NO:69; the VH4 CDR1 comprises an amino acid sequence of SEQ ID NO:75; the VH4 CDR2 comprises an amino acid sequence of SEQ ID NO:76; the VH4 CDR3 comprises an amino acid sequence of SEQ ID NO:77; the VL1 CDR1 comprises an amino acid sequence of SEQ ID NO:55; the VL1 CDR2 comprises an amino acid sequence of SEQ ID NO:56 (having the sequence of GTN); the VL1 CDR3 comprises an amino acid sequence of SEQ ID NO:57; the VL2 CDR1 comprises an amino acid sequence of SEQ ID NO:47; the VL2 CDR2 comprises an amino acid sequence of SEQ ID NO:48 (having the sequence of GAS); the VL2 CDR3 comprises an amino acid sequence of SEQ ID NO:49; the VL3 CDR1 comprises an amino acid sequence of SEQ ID NO:63; the VL3 CDR2 comprises an amino acid sequence of SEQ ID NO:64 (having the sequence of DAS); the VL3 CDR3 comprises an amino acid sequence of SEQ ID NO:65; the VL4 CDR1 comprises an amino acid sequence of SEQ ID NO:39; the VL4 CDR2 comprises an amino acid sequence of SEQ ID NO:40 (having the sequence of GAS); and the VL4 CDR3 comprises an amino acid sequence of SEQ ID NO:41. In some aspects, the linker between VL1 and VL2 is SEQ ID NO:6. In some aspects, the linker between VL2 and VH2 is SEQ ID NO:27. In some aspects, the linker between VH2 and VH1 is SEQ ID NO:6. In some aspects, the linker between VL3 and VL4 is SEQ ID NO:6. In some aspects, the linker between VL4 and VH4 is SEQ ID NO:27. In some aspects, the linker between VH4 and VH3 is SEQ ID NO:6.

In some aspects, the VH1 CDR1 of an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; the VH1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; the VH1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; the VH2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:179; the VH2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:180; the VH2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:181; the VH3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; the VH3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; the VH3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; the VH4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:179; the VH4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:180; the VH4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:181; the VL1 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; the VL1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); the VL1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; the VL2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:175; the VL2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:176; the VL2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:177; the VL3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; the VL3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); the VL3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; the VL4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:175; the VL4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:176; and the VL4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:177. In some aspects, the linker between VL1 and VL2 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL2 and VH2 has at least 80%, at least 85%, at least 90%, at least 91%, at least 92%, at least 93%, at least 94%, at least 95%, at least 96%, at least 97%, at least 98%, at least 99% or 100% identity to SEQ ID NO:27. In some aspects, the linker between VH2 and VH1 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL3 and VL4 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL4 and VH4 has at least 80%, at least 85%, at least 90%, at least 91%, at least 92%, at least 93%, at least 94%, at least 95%, at least 96%, at least 97%, at least 98%, at least 99% or 100% identity to SEQ ID NO:27. In some aspects, the linker between VH4 and VH3 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VH1 and/or VH3 and the Fc region has at least 80%, at least 85%, at least 90%, at least 91%, at least 92%, at least 93%, at least 94%, at least 95%, at least 96%, at least 97%, at least 98%, at least 99% or 100% identity to SEQ ID NO:12. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with one or more linker sequence as specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with one or more linker sequence as specified above.

In some aspects, the VH1 CDR1 of an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:67; the VH1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:68; the VH1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:69; the VH2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:179; the VH2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:180; the VH2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:181; the VH3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:67; the VH3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:68; the VH3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:69; the VH4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:179; the VH4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:180; the VH4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:181; the VL1 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:63; the VL1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:64 (having the sequence of DAS); the VL1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:65; the VL2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:175; the VL2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:176; the VL2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:177; the VL3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:63; the VL3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:64 (having the sequence of DAS); the VL3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:65; the VL4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:175; the VL4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:176; and the VL4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:177. In some aspects, the linker between VL1 and VL2 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL2 and VH2 has at least 80%, at least 85%, at least 90%, at least 91%, at least 92%, at least 93%, at least 94%, at least 95%, at least 96%, at least 97%, at least 98%, at least 99% or 100% identity to SEQ ID NO:27. In some aspects, the linker between VH2 and VH1 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL3 and VL4 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL4 and VH4 has at least 80%, at least 85%, at least 90%, at least 91%, at least 92%, at least 93%, at least 94%, at least 95%, at least 96%, at least 97%, at least 98%, at least 99% or 100% identity to SEQ ID NO:27. In some aspects, the linker between VH4 and VH3 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VH1 and/or VH3 and the Fc region has at least 80%, at least 85%, at least 90%, at least 91%, at least 92%, at least 93%, at least 94%, at least 95%, at least 96%, at least 97%, at least 98%, at least 99% or 100% identity to SEQ ID NO:12. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with one or more linker sequence as specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with one or more linker sequence as specified above.

In some aspects, the VH1 CDR1 of an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; the VH1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; the VH1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; the VH2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:171; the VH2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:172; the VH2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:173; the VH3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; the VH3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; the VH3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; the VH4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:171; the VH4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:172; the VH4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:173; the VL1 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; the VL1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); the VL1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; the VL2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:167; the VL2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:168; the VL2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:169; the VL3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; the VL3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); the VL3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; the VL4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:167; the VL4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:168; and the VL4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:169. In some aspects, the linker between VL1 and VL2 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL2 and VH2 has at least 80%, at least 85%, at least 90%, at least 91%, at least 92%, at least 93%, at least 94%, at least 95%, at least 96%, at least 97%, at least 98%, at least 99% or 100% identity to SEQ ID NO:27. In some aspects, the linker between VH2 and VH1 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL3 and VL4 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL4 and VH4 has at least 80%, at least 85%, at least 90%, at least 91%, at least 92%, at least 93%, at least 94%, at least 95%, at least 96%, at least 97%, at least 98%, at least 99% or 100% identity to SEQ ID NO:27. In some aspects, the linker between VH4 and VH3 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VH1 and/or VH3 and the Fc region has at least 80%, at least 85%, at least 90%, at least 91%, at least 92%, at least 93%, at least 94%, at least 95%, at least 96%, at least 97%, at least 98%, at least 99% or 100% identity to SEQ ID NO:12. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with one or more linker sequence as specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with one or more linker sequence as specified above.

In some aspects, the VH1 CDR1 of an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:63; the VH1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:64 (having the sequence of DAS); the VH1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:65; the VH2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:167; the VH2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:168; the VH2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:169; the VH3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:63; the VH3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:64 (having the sequence of DAS); the VH3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:65; the VH4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:167; the VH4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:168; the VH4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:169; the VL1 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:63; the VL1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:64 (having the sequence of DAS); the VL1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:65; the VL2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:167; the VL2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:168; the VL2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:169; the VL3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:63; the VL3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:64 (having the sequence of DAS); the VL3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:65; the VL4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:167; the VL4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:168; and the VL4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:169. In some aspects, the linker between VL1 and VL2 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL2 and VH2 has at least 80%, at least 85%, at least 90%, at least 91%, at least 92%, at least 93%, at least 94%, at least 95%, at least 96%, at least 97%, at least 98%, at least 99% or 100% identity to SEQ ID NO:27. In some aspects, the linker between VH2 and VH1 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL3 and VL4 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL4 and VH4 has at least 80%, at least 85%, at least 90%, at least 91%, at least 92%, at least 93%, at least 94%, at least 95%, at least 96%, at least 97%, at least 98%, at least 99% or 100% identity to SEQ ID NO:27. In some aspects, the linker between VH4 and VH3 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VH1 and/or VH3 and the Fc region has at least 80%, at least 85%, at least 90%, at least 91%, at least 92%, at least 93%, at least 94%, at least 95%, at least 96%, at least 97%, at least 98%, at least 99% or 100% identity to SEQ ID NO:12. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with one or more linker sequence as specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with one or more linker sequence as specified above.

In some aspects, the VH1 CDR1 of an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; the VH1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; the VH1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; the VH2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:179; the VH2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:180; the VH2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:181; the VH3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:179; the VH3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:180; the VH3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:181; the VH4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:67; the VH4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:68; the VH4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:69; the VL1 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; the VL1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); the VL1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; the VL2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:175; the VL2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:176; the VL2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:177; the VL3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:175; the VL3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:176; the VL3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:177; the VL4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:63; the VL4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:64 (having the sequence of DAS); and the VL4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:65. In some aspects, the linker between VL1 and VL2 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL2 and VH2 has at least 80%, at least 85%, at least 90%, at least 91%, at least 92%, at least 93%, at least 94%, at least 95%, at least 96%, at least 97%, at least 98%, at least 99% or 100% identity to SEQ ID NO:27. In some aspects, the linker between VH2 and VH1 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL3 and VL4 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL4 and VH4 has at least 80%, at least 85%, at least 90%, at least 91%, at least 92%, at least 93%, at least 94%, at least 95%, at least 96%, at least 97%, at least 98%, at least 99% or 100% identity to SEQ ID NO:27. In some aspects, the linker between VH4 and VH3 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VH1 and/or VH3 and the Fc region has at least 80%, at least 85%, at least 90%, at least 91%, at least 92%, at least 93%, at least 94%, at least 95%, at least 96%, at least 97%, at least 98%, at least 99% or 100% identity to SEQ ID NO:12. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with one or more linker sequence as specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with one or more linker sequence as specified above.

In some aspects, the VH1 CDR1 of an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; the VH1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; the VH1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; the VH2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:179; the VH2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:180; the VH2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:181; the VH3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:67; the VH3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:68; the VH3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:69; the VH4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:179; the VH4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:180; the VH4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:181; the VL1 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; the VL1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); the VL1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; the VL2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:175; the VL2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:176; the VL2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:177; the VL3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:63; the VL3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:64 (having the sequence of DAS); the VL3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:65; the VL4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:175; the VL4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:176; and the VL4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:177. In some aspects, the linker between VL1 and VL2 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL2 and VH2 has at least 80%, at least 85%, at least 90%, at least 91%, at least 92%, at least 93%, at least 94%, at least 95%, at least 96%, at least 97%, at least 98%, at least 99% or 100% identity to SEQ ID NO:27. In some aspects, the linker between VH2 and VH1 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL3 and VL4 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL4 and VH4 has at least 80%, at least 85%, at least 90%, at least 91%, at least 92%, at least 93%, at least 94%, at least 95%, at least 96%, at least 97%, at least 98%, at least 99% or 100% identity to SEQ ID NO:27. In some aspects, the linker between VH4 and VH3 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VH1 and/or VH3 and the Fc region has at least 80%, at least 85%, at least 90%, at least 91%, at least 92%, at least 93%, at least 94%, at least 95%, at least 96%, at least 97%, at least 98%, at least 99% or 100% identity to SEQ ID NO:12. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with one or more linker sequence as specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with one or more linker sequence as specified above.

In some aspects, the VH1 CDR1 of an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; the VH1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; the VH1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; the VH2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:171; the VH2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:172; the VH2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:173; the VH3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:171; the VH3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:172; the VH3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:173; the VH4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:67; the VH4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:68; the VH4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:69; the VL1 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; the VL1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); the VL1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; the VL2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:167; the VL2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:168; the VL2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:169; the VL3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:167; the VL3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:168; the VL3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:169; the VL4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:63; the VL4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:64 (having the sequence of DAS); and the VL4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:65. In some aspects, the linker between VL1 and VL2 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL2 and VH2 has at least 80%, at least 85%, at least 90%, at least 91%, at least 92%, at least 93%, at least 94%, at least 95%, at least 96%, at least 97%, at least 98%, at least 99% or 100% identity to SEQ ID NO:27. In some aspects, the linker between VH2 and VH1 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL3 and VL4 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL4 and VH4 has at least 80%, at least 85%, at least 90%, at least 91%, at least 92%, at least 93%, at least 94%, at least 95%, at least 96%, at least 97%, at least 98%, at least 99% or 100% identity to SEQ ID NO:27. In some aspects, the linker between VH4 and VH3 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VH1 and/or VH3 and the Fc region has at least 80%, at least 85%, at least 90%, at least 91%, at least 92%, at least 93%, at least 94%, at least 95%, at least 96%, at least 97%, at least 98%, at least 99% or 100% identity to SEQ ID NO:12. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with one or more linker sequence as specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with one or more linker sequence as specified above.

In some aspects, the VH1 CDR1 of an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; the VH1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; the VH1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; the VH2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:171; the VH2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:172; the VH2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:173; the VH3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:67; the VH3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:68; the VH3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:69; the VH4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:171; the VH4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:172; the VH4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:173; the VL1 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; the VL1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); the VL1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; the VL2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:167; the VL2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:168; the VL2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:169; the VL3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:63; the VL3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:64 (having the sequence of DAS); the VL3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:65; the VL4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:167; the VL4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:168; and the VL4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:169. In some aspects, the linker between VL1 and VL2 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL2 and VH2 has at least 80%, at least 85%, at least 90%, at least 91%, at least 92%, at least 93%, at least 94%, at least 95%, at least 96%, at least 97%, at least 98%, at least 99% or 100% identity to SEQ ID NO:27. In some aspects, the linker between VH2 and VH1 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL3 and VL4 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL4 and VH4 has at least 80%, at least 85%, at least 90%, at least 91%, at least 92%, at least 93%, at least 94%, at least 95%, at least 96%, at least 97%, at least 98%, at least 99% or 100% identity to SEQ ID NO:27. In some aspects, the linker between VH4 and VH3 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VH1 and/or VH3 and the Fc region has at least 80%, at least 85%, at least 90%, at least 91%, at least 92%, at least 93%, at least 94%, at least 95%, at least 96%, at least 97%, at least 98%, at least 99% or 100% identity to SEQ ID NO:12. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally one or more linker sequence as specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with one or more linker sequence as specified above.

In some aspects, the VH1 CDR1 of an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:183; the VH1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:184; the VH1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:185; and/or the VH2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:183; the VH2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:184; the VH2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:185; and/or the VH3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:183; the VH3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:184; the VH3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:185; and/or the VH4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:183; the VH4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:184; the VH4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:185; and/or the VL1 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:187; the VL1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:188; the VL1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:189; and/or the VL2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:187; the VL2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:188; the VL2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:189; and/or the VL3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:187; the VL3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:188; the VL3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:189; and/or the VL4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:187; the VL4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:188; and the VL4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:189. In some aspects, the linker between VL1 and VL2 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL2 and VH2 has at least 80%, at least 85%, at least 90%, at least 91%, at least 92%, at least 93%, at least 94%, at least 95%, at least 96%, at least 97%, at least 98%, at least 99% or 100% identity to SEQ ID NO:27. In some aspects, the linker between VH2 and VH1 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL3 and VL4 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL4 and VH4 has at least 80%, at least 85%, at least 90%, at least 91%, at least 92%, at least 93%, at least 94%, at least 95%, at least 96%, at least 97%, at least 98%, at least 99% or 100% identity to SEQ ID NO:27. In some aspects, the linker between VH4 and VH3 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VH1 and/or VH3 and the Fc region has at least 80%, at least 85%, at least 90%, at least 91%, at least 92%, at least 93%, at least 94%, at least 95%, at least 96%, at least 97%, at least 98%, at least 99% or 100% identity to SEQ ID NO:12. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with one or more linker sequence as specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally one or more linker sequence as specified above.

In some aspects, the VH1 CDR1 of an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:183; the VH1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:184; the VH1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:185; the VH2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:179; the VH2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:180; the VH2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:181; the VH3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:179; the VH3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:180; the VH3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:181; the VH4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:183; the VH4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:184; the VH4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:185; the VL1 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:187; the VL1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:188; the VL1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:189; the VL2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:175; the VL2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:176; the VL2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:177; the VL3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:175; the VL3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:176; the VL3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:177; the VL4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:187; the VL4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:188; and the VL4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:189. In some aspects, the linker between VL1 and VL2 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL2 and VH2 has at least 80%, at least 85%, at least 90%, at least 91%, at least 92%, at least 93%, at least 94%, at least 95%, at least 96%, at least 97%, at least 98%, at least 99% or 100% identity to SEQ ID NO:27. In some aspects, the linker between VH2 and VH1 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL3 and VL4 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL4 and VH4 has at least 80%, at least 85%, at least 90%, at least 91%, at least 92%, at least 93%, at least 94%, at least 95%, at least 96%, at least 97%, at least 98%, at least 99% or 100% identity to SEQ ID NO:27. In some aspects, the linker between VH4 and VH3 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VH1 and/or VH3 and the Fc region has at least 80%, at least 85%, at least 90%, at least 91%, at least 92%, at least 93%, at least 94%, at least 95%, at least 96%, at least 97%, at least 98%, at least 99% or 100% identity to SEQ ID NO:12. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally one or more linker sequence as specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with one or more linker sequence as specified above.

In some aspects, the VH1 CDR1 of an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:183; the VH1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:184; the VH1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:185; the VH2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:171; the VH2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:172; the VH2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:173; the VH3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:171; the VH3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:172; the VH3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:173; the VH4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:183; the VH4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:184; the VH4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:185; the VL1 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:187; the VL1 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:188; the VL1 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:189; the VL2 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:167; the VL2 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:168; the VL2 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:169; the VL3 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:167; the VL3 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:168; the VL3 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:169; the VL4 CDR1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:187; the VL4 CDR2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:188; and the VL4 CDR3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:189. In some aspects, the linker between VL1 and VL2 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL2 and VH2 has at least 80%, at least 85%, at least 90%, at least 91%, at least 92%, at least 93%, at least 94%, at least 95%, at least 96%, at least 97%, at least 98%, at least 99% or 100% identity to SEQ ID NO:27. In some aspects, the linker between VH2 and VH1 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL3 and VL4 has at least 80%, at least 85%, at least 90%, at least 91%, at least 92%, at least 93%, at least 94%, at least 95%, at least 96%, at least 97%, at least 98%, at least 99% or 100% identity to SEQ ID NO:6. In some aspects, the linker between VL4 and VH4 has at least 80%, at least 85%, at least 90%, at least 91%, at least 92%, at least 93%, at least 94%, at least 95%, at least 96%, at least 97%, at least 98%, at least 99% or 100% identity to SEQ ID NO:27. In some aspects, the linker between VH4 and VH3 has at least 80%, at least 85%, at least 90%, at least 95%, at least 99% or 100% identity to SEQ ID NO:6. In some aspects, the linker between VH1 and/or VH3 and the Fc region has at least 80%, at least 85%, at least 90%, at least 95%, at least 99% or 100% identity to SEQ ID NO:12. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with one or more linker sequence as specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally one or more linker sequence as specified above.

In some aspects, VH1 of an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:58; VH2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:178; VH3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:58; VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:178; VL1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:54; VL2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:174; VL3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:54; and VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:174. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above.

In some aspects, VH1 of an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:66; VH2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:178; VH3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:66; VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:178; VL1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:62; VL2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:174; VL3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:62; and VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:174. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above.

In some aspects, VH1 of an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:58; VH2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:170; VH3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:58; VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:170; VL1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:54; VL2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:166; VL3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:54; and VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:166. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above.

In some aspects, VH1 of an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:66; VH2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:170; VH3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:66; VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:170; VL1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:62; VL2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:166; VL3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:62; and VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:166. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above.

In some aspects, VH1 of an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:58; VH2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:178; VH3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:178; VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:66; VL1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:54; VL2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:174; VL3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:174; and VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:62. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above.

In some aspects, VH1 of an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:58; VH2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:178; VH3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:66; VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:178; VL1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:54; VL2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:174; VL3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:62; and VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:174. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above.

In some aspects, VH1 of an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:58; VH2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:170; VH3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:170; VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:66; VL1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:54; VL2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:166; VL3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:166; and VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:62. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above.

In some aspects, VH1 of an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:58; VH2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:170; VH3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:66; VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:170; VL1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:54; VL2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:166; VL3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:62; and VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:166. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above.

In some aspects, VH1 of an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:182; and/or VH2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:182; and/or VH3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:182; and/or VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:182; and/or VL1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:186; and/or VL2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:186; and/or VL3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:186; and/or VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:186. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above.

In some aspects, VH1 of an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:182; VH2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:178; VH3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:178; VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:182; VL1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:186; VL2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:174; VL3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:174; and VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:186. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above.

In some aspects, VH1 of an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:182; VH2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:170; VH3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:170; VH4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:182; VL1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:186; VL2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:166; VL3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:166; and VL4 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:186. In some preferred aspects, VH1, VH2, VH3, VH4, VL1, VL2, VL3 and VL4 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VH4, VL1, VL2, VL3 and VL4 comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:190, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:190. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:191, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:191. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:192, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:192. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:193, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:193. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:194, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:195. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:196, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:197. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:198, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:199. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:200, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:201. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide or antigen binding polypeptide complex provided herein comprises a polypeptide having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:202-258, 291, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505 and 711-754. In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:202-258, 291, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505 and 711-754, and a second polypeptide having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:202-258, 291, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505 and 711-754. In some preferred aspects, the first polypeptide and second polypeptide have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of a corresponding amino acid sequence specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:202, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:202. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:203, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:203. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:204, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:204. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:205, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:205. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:206, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:206. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:207, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:208. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:209, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:209. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:210, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:211. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:212, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:213. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:214, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:215. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:216, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:217. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:218, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:219. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:220, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:221. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:222, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:223. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:224, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:225. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:226, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:227. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:228, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:228. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:229, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:229. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:230, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:231. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:232, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:233. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:234, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:235. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:236, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:237. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:238, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:239. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:240, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:241. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:242, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:243. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:244, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:245. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:246, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:247. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:248, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:249. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:250, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:251. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:252, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:253. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:254, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:255. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide or antigen binding polypeptide complex provided herein comprises a polypeptide having a structure represented by VL1-L1-VL2-L2-VH2-L3-VH1-L4-Fc-L5-Fc; VL.1-L1-VH2-L2-VL2-L3-VH1-L4-Fc-L5-Fc; VH1-L6-VH2-L7-VL2-L8-VL1-L9-Fc-L10-Fc; or VH1-L6-VL2-L7-VH2-L8-VL.1-L9-Fc-L10-Fc; wherein VL1 is a first immunoglobulin light chain variable region that specifically binds to a SARS-CoV-2 protein; VL2 is a second immunoglobulin light chain variable region that specifically binds a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; Fc is a region comprising an immunoglobulin heavy chain constant region 2 (CH2), an immunoglobulin heavy chain constant region 3 (CH3), and optionally, an immunoglobulin hinge; and L1-L10 are amino acid linkers.

In some aspects, an antigen binding polypeptide or antigen binding polypeptide complex provided herein comprises a polypeptide having a structure represented by VL1-L1-VL2-L2-VH2-L3-VH1-L4-Fc-L5-Fc or VH1-L6-VH2-L7-VL2-L8-VL1-L9-Fc-L10-Fc; wherein VL1 is a first immunoglobulin light chain variable region that specifically binds to a SARS-CoV-2 protein; VL2 is a second immunoglobulin light chain variable region that specifically binds a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; Fc is a region comprising an immunoglobulin heavy chain constant region 2 (CH2), an immunoglobulin heavy chain constant region 3 (CH3), and optionally, an immunoglobulin hinge; and L1-L10 are amino acid linkers.

In some aspects, an antigen binding polypeptide or antigen binding polypeptide complex provided herein comprises a polypeptide having a structure represented by VL1-L1-VH2-L2-VL2-L3-VH1-L4-Fc-L5-Fc or VH1-L6-VL2-L7-VH2-L8-VL1-L9-Fc-L10-Fc; wherein VL1 is a first immunoglobulin light chain variable region that specifically binds to a SARS-CoV-2 protein; VL2 is a second immunoglobulin light chain variable region that specifically binds a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; Fc is a region comprising an immunoglobulin heavy chain constant region 2 (CH2), an immunoglobulin heavy chain constant region 3 (CH3), and optionally, an immunoglobulin hinge; and L1-L10 are amino acid linkers.

In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:78-113, 150-165, 190-258, 291, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505 and 711-754. In some preferred aspects, the antigen binding polypeptide or antigen binding polypeptide complex comprises an amino acid having at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the antigen binding polypeptide or antigen binding polypeptide complex comprises or consists of a corresponding amino acid sequence specified above.

In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:78. In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:78, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:78. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:79. In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:79, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:79. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:80. In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:80, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:80. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:81. In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:81, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:81. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:82. In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:82, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:82. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:83. In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:83, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:83. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:84. In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:84, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:84. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:85. In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:85, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:85. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:86. In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:86, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:86. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:87. In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:87, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:87. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:88. In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:88, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:88. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:89. In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:89, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:89. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:90. In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:90, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:90. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:91. In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:91, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:91. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:92. In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:92, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:92. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:93. In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:93, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:93. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:94. In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:94, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:94. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:95. In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:95, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:95. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:96. In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:96, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:96. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:97. In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:97, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:97. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:98. In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:99. In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:98, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:99. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:100. In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:101. In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:100, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:101. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:102. In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:103. In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:102, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:103. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:104. In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:105. In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:104, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:105. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:106. In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:107. In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:106, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:107. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:108. In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:109. In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:108, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:109. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:110. In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:111. In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:110, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:111. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:112. In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:113. In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:112, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:113. In some aspects, an antigen binding polypeptide complex provided herein comprises a first polypeptide having an amino acid sequence of SEQ ID NO:112, and a second polypeptide having an amino acid sequence of SEQ ID NO:113. In some preferred aspects, the first polypeptide and second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and second polypeptide comprise or consist of the corresponding amino acid sequences specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:114-150, 292, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 393, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 507, 671-710 and 755-777. In some preferred aspects, the polynucleotide has at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the polynucleotide comprises or consists of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:114. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:114, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:114. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:115. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:115, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:115. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:116. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:116, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:116. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:117. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:117, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:117. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:118. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:118, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:118. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:119. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:119, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:119. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:120. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:120, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:120. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:121. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:121, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:121. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:122. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:122, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:122. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:123. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:123, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:123. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:124. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:124, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:124. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:125. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:125, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:125. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:126. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:126, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:126. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:127. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:127, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:127. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:128. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:128, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:128. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:129. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:129, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:129. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:130. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:130, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:130. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:131. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:131, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:131. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:132. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:132, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:132. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:133. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:133, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:133. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:134. In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:135. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:134, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:135. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:136. In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:137. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:136, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:137. In some preferred aspects, the first polypeptide and the second polypeptide are each encoded by a polynucleotide having at least 90% identity with the corresponding nucleic acid acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of the corresponding nucleic acid sequences specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:138. In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:139. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:138, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:139. In some preferred aspects, the first polypeptide and the second polypeptide are each encoded by a polynucleotide having at least 90% identity with the corresponding nucleic acid acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of the corresponding nucleic acid sequences specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:140. In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:141. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:140, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:141. In some preferred aspects, the first polypeptide and the second polypeptide are each encoded by a polynucleotide having at least 90% identity with the corresponding nucleic acid acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of the corresponding nucleic acid sequences specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:142. In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:143. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:142, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:143. In some preferred aspects, the first polypeptide and the second polypeptide are each encoded by a polynucleotide having at least 90% identity with the corresponding nucleic acid acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of the corresponding nucleic acid sequences specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:144. In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:145. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:144, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:145. In some preferred aspects, the first polypeptide and the second polypeptide are each encoded by a polynucleotide having at least 90% identity with the corresponding nucleic acid acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of the corresponding nucleic acid sequences specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:146. In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:147. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:146, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:147. In some preferred aspects, the first polypeptide and the second polypeptide are each encoded by a polynucleotide having at least 90% identity with the corresponding nucleic acid acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of the corresponding nucleic acid sequences specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:148. In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:149. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:148, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:149. In some aspects, an antigen binding polypeptide complex provided herein comprises a first polypeptide encoded by the polynucleotide of SEQ ID NO:148, and a second polypeptide encoded by the polynucleotide of SEQ ID NO:149. In some preferred aspects, the first polypeptide and the second polypeptide are each encoded by a polynucleotide having at least 90% identity with the corresponding nucleic acid acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of the corresponding nucleic acid sequences specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:671 or 672. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:671 or 672, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:671 or 672. In some aspects, an antigen binding polypeptide complex provided herein comprises a first polypeptide encoded by the polynucleotide of SEQ ID NO:671 or 672, and a second polypeptide encoded by the polynucleotide of SEQ ID NO:671 or 672. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:673 or 674. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:673 or 674, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:673 or 674. In some aspects, an antigen binding polypeptide complex provided herein comprises a first polypeptide encoded by the polynucleotide of SEQ ID NO:673 or 674, and a second polypeptide encoded by the polynucleotide of SEQ ID NO:673 or 674. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:675 or 676. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:675 or 676, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:675 or 676. In some aspects, an antigen binding polypeptide complex provided herein comprises a first polypeptide encoded by the polynucleotide of SEQ ID NO:675 or 676, and a second polypeptide encoded by the polynucleotide of SEQ ID NO:675 or 676 In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:677 or 678. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:677 or 678, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:677 or 678. In some aspects, an antigen binding polypeptide complex provided herein comprises a first polypeptide encoded by the polynucleotide of SEQ ID NO:677 or 678, and a second polypeptide encoded by the polynucleotide of SEQ ID NO:677 or 678. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:679 or 680. In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:681 or 682. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:679 or 680, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:681 or 682. In some aspects, an antigen binding polypeptide complex provided herein comprises a first polypeptide encoded by the polynucleotide of SEQ ID NO:679 or 680, and a second polypeptide encoded by the polynucleotide of SEQ ID NO:681 or 682. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:683 or 684. In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:685 or 686. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:683 or 684, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:685 or 686. In some aspects, an antigen binding polypeptide complex provided herein comprises a first polypeptide encoded by the polynucleotide of SEQ ID NO:683 or 684, and a second polypeptide encoded by the polynucleotide of SEQ ID NO:685 or 686. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:687 or 688. In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:689 or 690. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:687 or 688, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:689 or 690. In some aspects, an antigen binding polypeptide complex provided herein comprises a first polypeptide encoded by the polynucleotide of SEQ ID NO:687 or 688, and a second polypeptide encoded by the polynucleotide of SEQ ID NO:689 or 690. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:691 or 692. In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:693 or 694. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:691 or 692, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:693 or 694. In some aspects, an antigen binding polypeptide complex provided herein comprises a first polypeptide encoded by the polynucleotide of SEQ ID NO:691 or 692, and a second polypeptide encoded by the polynucleotide of SEQ ID NO:693 or 694. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:695 or 696. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:695 or 696, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:695 or 696. In some aspects, an antigen binding polypeptide complex provided herein comprises a first polypeptide encoded by the polynucleotide of SEQ ID NO:695 or 696, and a second polypeptide encoded by the polynucleotide of SEQ ID NO:695 or 696. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:697 or 698. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:697 or 698, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:697 or 698. In some aspects, an antigen binding polypeptide complex provided herein comprises a first polypeptide encoded by the polynucleotide of SEQ ID NO:697 or 698, and a second polypeptide encoded by the polynucleotide of SEQ ID NO:697 or 698. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:699 or 700. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:699 or 700, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:699 or 700. In some aspects, an antigen binding polypeptide complex provided herein comprises a first polypeptide encoded by the polynucleotide of SEQ ID NO:699 or 700, and a second polypeptide encoded by the polynucleotide of SEQ ID NO:699 or 700. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:701 or 702. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:701 or 702, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:701 or 702. In some aspects, an antigen binding polypeptide complex provided herein comprises a first polypeptide encoded by the polynucleotide of SEQ ID NO:701 or 702, and a second polypeptide encoded by the polynucleotide of SEQ ID NO:701 or 702. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:703 or 704. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:703 or 704, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:703 or 704. In some aspects, an antigen binding polypeptide complex provided herein comprises a first polypeptide encoded by the polynucleotide of SEQ ID NO:703 or 704, and a second polypeptide encoded by the polynucleotide of SEQ ID NO:703 or 704. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:705 or 706. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:705 or 706, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:705 or 706. In some aspects, an antigen binding polypeptide complex provided herein comprises a first polypeptide encoded by the polynucleotide of SEQ ID NO:705 or 706, and a second polypeptide encoded by the polynucleotide of SEQ ID NO:705 or 706. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:707 or 708. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:707 or 704, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:707 or 708. In some aspects, an antigen binding polypeptide complex provided herein comprises a first polypeptide encoded by the polynucleotide of SEQ ID NO:707 or 708, and a second polypeptide encoded by the polynucleotide of SEQ ID NO:707 or 708. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:709 or 710. In some aspects, an antigen binding polypeptide complex provided herein comprises a first polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:709 or 710, and a second polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:709 or 710. In some aspects, an antigen binding polypeptide complex provided herein comprises a first polypeptide encoded by the polynucleotide of SEQ ID NO:709 or 710, and a second polypeptide encoded by the polynucleotide of SEQ ID NO:709 or 710. In some preferred aspects, the first polypeptide and the second polypeptide are each encoded by a polynucleotide having at least 90% identity with a corresponding nucleic acid acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide are each encoded by a polynucleotide comprising or consisting of a corresponding nucleic acid sequence specified above.

In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:256. In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:256, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:256. In some preferred aspects, the first polypeptide and the second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide comprise or consist of the corresponding amino acid sequences specified above.

In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:257. In some aspects, an antigen binding polypeptide or antigen binding polypeptide complex provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:258. In some aspects, an antigen binding polypeptide complex provided herein comprises a first polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:257, and a second polypeptide having at least 80%, at least 85%, at least 90%, at least 91%, at least 92%, at least 93%, at least 94%, at least 95%, at least 96%, at least 97%, at least 98%, at least 99% or 100% identity to SEQ ID NO:258. In some preferred aspects, the first polypeptide and the second polypeptide have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, the first polypeptide and the second polypeptide comprise or consist of the corresponding amino acid sequences specified above.

In some aspects, provided herein is an antigen binding polypeptide complex comprising a first polypeptide and a second polypeptide; wherein the first polypeptide has a structure represented by VL1-L1-VH1 or VH1-L2-VL1; wherein the second polypeptide has a structure represented by VL2-L3-VH2 or VH2-L4-VL2; wherein VL1 is a first immunoglobulin light chain variable region that specifically binds to a SARS-CoV-2 protein; VH1 is a first immunoglobulin heavy chain variable region that specifically binds to a SARS-CoV-2 protein; wherein VL2 is a second immunoglobulin light chain variable region that specifically binds to a SARS-CoV-2 protein; wherein VH2 is a second immunoglobulin heavy chain variable region that specifically binds to a SARS-CoV-2 protein; and L1-L4 are amino acid linkers. In some aspects, provided herein is an antigen binding polypeptide complex comprising a first polypeptide and a second polypeptide; wherein the first polypeptide has a structure represented by VL1-L1-VH1-L2-Fc or VH1-L3-VL1-L4-Fc; wherein the second polypeptide has a structure represented by VL2-L5-VH2-L6-Fc or VH2-L7-VL2-L8-Fc; wherein VL1 is a first immunoglobulin light chain variable region that specifically binds to a SARS-CoV-2 protein; VH1 is a first immunoglobulin heavy chain variable region that specifically binds to a SARS-CoV-2 protein; wherein VL2 is a second immunoglobulin light chain variable region that specifically binds to a SARS-CoV-2 protein; wherein VH2 is a second immunoglobulin heavy chain variable region that specifically binds to a SARS-CoV-2 protein; L1-L8 are amino acid linkers; and Fc is a region comprising an immunoglobulin heavy chain constant region 2 (CH2), an immunoglobulin heavy chain constant region 3 (CH3), and optionally, an immunoglobulin hinge. For example, the first polypeptide has a structure represented by VL1-L1-VH1 and the second polypeptide has a structure represented by VL2-L3-VH2 or VH2-L4-VL2. For example, the first polypeptide has a structure represented by VH1-L2-VL1 and the second polypeptide has a structure represented by VL2-L3-VH2 or VH2-L4-VL2.

In some aspects, VH1 and VH2 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:264; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:265; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:266; and VL1 and VL2 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:260; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:261; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:262. In some aspects, VH1 and VH2 comprise a CDR1 comprising the amino acid sequence of SEQ ID NO:264; a CDR2 comprising the amino acid sequence of SEQ ID NO:265; and a CDR3 comprising the amino acid sequence of SEQ ID NO:266; and VL1 and VL2 comprise a CDR1 comprising the amino acid sequence of SEQ ID NO:260; a CDR2 comprising the amino acid sequence of SEQ ID NO:261; and a CDR3 comprising the amino acid sequence of SEQ ID NO:262. In some aspects, L1 or L2 and L3 or L4 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L2 and L3 or L4 comprise the amino acid sequence of SEQ ID NO:27. In some aspects, L1 or L3 and L5 or L7 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L3 and L5 or L7 comprise the amino acid sequence of SEQ ID NO:27. In some aspects, L2 or L4 and L6 or L8 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L2 or L4 and L6 or L8 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some preferred aspects, CDR1, CDR2 and CDR3 of each of VH1, VH2, VL1 and VL2 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VL1 and VL2 comprise or consist of the corresponding amino acid sequences specified above, optionally with (i) an Fc comprising or consisting of the corresponding amino acid sequence recited above; and/or (ii) one or more linker sequence as specified above.

In some aspects, VH1 and VH2 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:272; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:273; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:274; and VL1 and VL2 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:268; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:269 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:270. In some aspects, VH1 and VH2 comprise a CDR1 comprising the amino acid sequence of SEQ ID NO:272; a CDR2 comprising the amino acid sequence of SEQ ID NO:273; and a CDR3 comprising the amino acid sequence of SEQ ID NO:274; and VL1 and VL2 comprise a CDR1 comprising the amino acid sequence of SEQ ID NO:268; a CDR2 comprising the amino acid sequence of SEQ ID NO:269 (having the sequence of GAS); and a CDR3 comprising the amino acid sequence of SEQ ID NO:270. In some aspects, L1 or L2 and L3 or L4 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L2 and L3 or L4 comprise the amino acid sequence of SEQ ID NO:27. In some aspects, L1 or L3 and L5 or L7 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L3 and L5 or L7 comprise the amino acid sequence of SEQ ID NO:27. In some aspects, L2 or L4 and L6 or L8 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L2 or L4 and L6 or L8 comprise the amino acid sequenee of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some preferred aspects, CDR1, CDR2 and CDR3 of each of VH1, VH2, VL1 and VL2 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VL1 and VL2 comprise or consist of the corresponding amino acid sequences specified above, optionally with (i) an Fc comprising or consisting of the corresponding amino acid sequence recited above; and/or (ii) one or more linker sequence as specified above.

In some aspects, VH1 and VH2 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; and VL1 and VL2 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, VH1 and VH2 comprise a CDR1 comprising the amino acid sequence of SEQ ID NO:280; a CDR2 comprising the amino acid sequence of SEQ ID NO:281; and a CDR3 comprising the amino acid sequence of SEQ ID NO:282; and VL1 and VL2 comprise a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1 or L2 and L3 or L4 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L2 and L3 or L4 comprise the amino acid sequence of SEQ ID NO:27. In some aspects, L1 or L3 and L5 or L7 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L3 and L5 or L7 comprise the amino acid sequence of SEQ ID NO:27. In some aspects, L2 or L4 and L6 or L8 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L2 or L4 and L6 or L8 comprise the amino acid sequenee of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some preferred aspects, CDR1, CDR2 and CDR3 of each of VH1, VH2, VL1 and VL2 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VL1 and VL2 comprise or consist of the corresponding amino acid sequences specified above, optionally with (i) an Fc comprising or consisting of the corresponding amino acid sequence recited above; and/or (ii) one or more linker sequence as specified above.

In some aspects, provided herein is an antigen binding polypeptide complex comprising a first polypeptide and a second polypeptide; wherein the first polypeptide has a structure represented by VL1-L1-VL2-L2-VH2-L3-VH1; VL1-L1-VH2-L2-VL2-L3-VH1; VH1-L4-VH2-L5-VL2-L6-VL1; or VH1-L4-VL2-L5-VH2-L6-VL1; wherein the second polypeptide has a structure represented by VL3-L7-VL4-L8-VH4-L9-VH3; VL3-L7-VH4-L8-VL4-L9-VH3; VH3-L10-VH4-L11-VL4-L12-VL3; or VH3-L10-VL4-L11-VH4-L12-VL3; wherein VL1 is a first immunoglobulin light chain variable region that specifically binds to a SARS-CoV-2 protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; and L1-L12 are amino acid linkers. In some aspects, provided herein is an antigen binding polypeptide complex comprising a first polypeptide and a second polypeptide; wherein the first polypeptide has a structure represented by VL1-L1-VL2-L2-VH2-L3-VH1-L4-Fc; VL1-L1-VH2-L2-VL2-L3-VH1-L4-Fc; VH1-L5-VH2-L6-VL2-L7-VL1-L8-Fc; or VH1-L5-VL2-L6-VH2-L7-VL1-L8-Fc; wherein the second polypeptide has a structure represented by Fc; VL3-L9-VL4-L10-VH4-L11-VH3-L12-Fc; VL3-L9-VH4-L10-VL4-L11-VH3-L12-Fc; VH3-L13-VH4-L14-VL4-L15-VL3-L16-Fc; or VH3-L13-VL4-L14-VH4-L15-VL3-L16-Fc; wherein VL1 is a first immunoglobulin light chain variable region that specifically binds to a SARS-CoV-2 spike protein; VL2 is a second immunoglobulin light chain variable region that specifically binds to a SARS-CoV-2 spike protein; VL3 is a third immunoglobulin light chain variable region that specifically binds to a SARS-CoV-2 spike protein; VL4 is a fourth immunoglobulin light chain variable region that specifically binds to a SARS-CoV-2 spike protein; VH1 is a first immunoglobulin heavy chain variable region that specifically binds to a SARS-CoV-2 spike protein; VH2 is a second immunoglobulin heavy chain variable region that specifically binds to a SARS-CoV-2 spike protein; VH3 is a third immunoglobulin heavy chain variable region that specifically binds to a SARS-CoV-2 spike protein; VH4 is a fourth immunoglobulin heavy chain variable region that specifically binds to a SARS-CoV-2 spike protein; Fc is a region comprising an immunoglobulin heavy chain constant region 2 (CH2), an immunoglobulin heavy chain constant region 3 (CH3), and optionally, an immunoglobulin hinge; and L1-L16 are amino acid linkers. In some aspects, provided herein is an antigen binding polypeptide complex comprising a first polypeptide and a second polypeptide; wherein the first polypeptide has a structure represented by VL1-L1-VL2-L2-VH2-L3-VH1; the second polypeptide has a structure represented by VL3-L7-VL4-L8-VH4-L9-VH3; VL3-L7-VH4-L8-VL4-L9-VH3; VH3-L10-VH4-L11-VL4-L12-VL3; or VH3-L10-VL4-L11-VH4-L12-VL3. In some aspects, provided herein is an antigen binding polypeptide complex comprising a first polypeptide and a second polypeptide; wherein the first polypeptide has a structure represented by VL1-L1-VH2-L2-VL2-L3-VH1; the second polypeptide has a structure represented by VL3-L7-VL4-L8-VH4-L9-VH3; VL3-L7-VH4-L8-VL4-L9-VH3; VH3-L10-VH4-L11-VL4-L12-VL3; or VH3-L10-VL4-L11-VH4-L12-VL3. In some aspects, provided herein is an antigen binding polypeptide complex comprising a first polypeptide and a second polypeptide; wherein the first polypeptide has a structure represented by VH1-L4-VH2-L5-VL2-L6-VL1; the second polypeptide has a structure represented by VL3-L7-VL4-L8-VH4-L9-VH3; VL3-L7-VH4-L8-VL4-L9-VH3; VH3-L10-VH4-L11-VL4-L12-VL3; or VH3-L10-VL4-L11-VH4-L12-VL3. In some aspects, provided herein is an antigen binding polypeptide complex comprising a first polypeptide and a second polypeptide; wherein the first polypeptide has a structure represented by VH1-L4-VL2-L5-VH2-L6-VL1; the second polypeptide has a structure represented by VL3-L7-VL4-L8-VH4-L9-VH3; VL3-L7-VH4-L8-VL4-L9-VH3; VH3-L10-VH4-L11-VL4-L12-VL3; or VH3-L10-VL4-L11-VH4-L12-VL3.

In some aspects, VH1, VH2, VH3 and VH4 each comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:264; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:265; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:266; and VL1, VL2, VL3 and VL4 each comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:260; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:261; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:262. In some aspects, VH1, VH2, VH3 and VH4 each comprise a CDR1 comprising the amino acid sequence of SEQ ID NO:264; a CDR2 comprising the amino acid sequence of SEQ ID NO:265; and a CDR3 comprising the amino acid sequence of SEQ ID NO:266; and VL1, VL2, VL3 and VL4 each comprise a CDR1 comprising the amino acid sequence of SEQ ID NO:260; a CDR2 comprising the amino acid sequence of SEQ ID NO:261; and a CDR3 comprising the amino acid sequence of SEQ ID NO:262. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, VH1, VH2, VH3 and VH4 each comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:272; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:273; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:274; and VL1, VL2, VL3 and VL4 each comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:268; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:269 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:270. In some aspects, VH1, VH2, VH3 and VH4 each comprise a CDR1 comprising the amino acid sequence of SEQ ID NO:272; a CDR2 comprising the amino acid sequence of SEQ ID NO:273; and a CDR3 comprising the amino acid sequence of SEQ ID NO:274; and VL1, VL2, VL3 and VL4 each comprise a CDR1 comprising the amino acid sequence of SEQ ID NO:268; a CDR2 comprising the amino acid sequence of SEQ ID NO:269 (having the sequence of GAS); and a CDR3 comprising the amino acid sequence of SEQ ID NO:270. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, VH1, VH2, VH3 and VH4 each comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; and VL1, VL2, VL3 and VL4 each comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, VH1, VH2, VH3 and VH4 each comprise a CDR1 comprising the amino acid sequence of SEQ ID NO:280; a CDR2 comprising the amino acid sequence of SEQ ID NO:281; and a CDR3 comprising the amino acid sequence of SEQ ID NO:282; and VL1, VL2, VL3 and VL4 each comprise a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (ii) VH2 and VH4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:264; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:265; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:266; (iii) VL1 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; and (iv) VL2 and VL4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:260; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:261; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:262. In some aspects, (i) VH1 and VH3 comprise a CDR1 comprising the amino acid sequence of SEQ ID NO:59; a CDR2 comprising the amino acid sequence of SEQ ID NO:60; and a CDR3 comprising the amino acid sequence of SEQ ID NO:61; (ii) VH2 and VH4 comprise a CDR1 comprising the amino acid sequence of SEQ ID NO:264; a CDR2 comprising the amino acid sequence of SEQ ID NO:265; and a CDR3 comprising the amino acid sequence of SEQ ID NO:266; (iii) VL1 and VL3 comprise a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; and (iv) VL2 and VL4 comprise a CDR1 comprising the amino acid sequence of SEQ ID NO:260; a CDR2 comprising the amino acid sequence of SEQ ID NO:261; and a CDR3 comprising the amino acid sequence of SEQ ID NO:262. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (ii) VH2 and VH4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:264; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:265; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:266; (iii) VL1 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278; and (iv) VL2 and VL4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:260; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:261; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:262. In some aspects, (i) VH1 and VH3 comprise a CDR1 comprising the amino acid sequence of SEQ ID NO:280; a CDR2 comprising the amino acid sequence of SEQ ID NO:281; and a CDR3 comprising the amino acid sequence of SEQ ID NO:282; (ii) VH2 and VH4 comprise a CDR1 comprising the amino acid sequence of SEQ ID NO:264; a CDR2 comprising the amino acid sequence of SEQ ID NO:265; and a CDR3 comprising the amino acid sequence of SEQ ID NO:266; (iii) VL1 and VL3 comprise a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278; and (iv) VL2 and VL4 comprise a CDR1 comprising the amino acid sequence of SEQ ID NO:260; a CDR2 comprising the amino acid sequence of SEQ ID NO:261; and a CDR3 comprising the amino acid sequence of SEQ ID NO:262. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:264; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:265; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:266; (ii) VH2 and VH4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (iii) VL1 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:260; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:261; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:262; and (iv) VL2 and VL4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57. In some aspects, (i) VH1 and VH3 comprise a CDR1 comprising the amino acid sequence of SEQ ID NO:264; a CDR2 comprising the amino acid sequence of SEQ ID NO:265; and a CDR3 comprising the amino acid sequence of SEQ ID NO:266; (ii) VH2 and VH4 comprise a CDR1 comprising the amino acid sequence of SEQ ID NO:59; a CDR2 comprising the amino acid sequence of SEQ ID NO:60; and a CDR3 comprising the amino acid sequence of SEQ ID NO:61; (iii) VL1 and VL3 comprise a CDR1 comprising the amino acid sequence of SEQ ID NO:260; a CDR2 comprising the amino acid sequence of SEQ ID NO:261; and a CDR3 comprising the amino acid sequence of SEQ ID NO:262; and (iv) VL2 and VL4 comprise a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:264; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:265; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:266; (ii) VH2 and VH4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iii) VL1 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:260; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:261; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:262; and (iv) VL2 and VL4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, (i) VH1 and VH3 comprise a CDR1 comprising the amino acid sequence of SEQ ID NO:264; a CDR2 comprising the amino acid sequence of SEQ ID NO:265; and a CDR3 comprising the amino acid sequence of SEQ ID NO:266; (ii) VH2 and VH4 comprise a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iii) VL1 and VL3 comprise a CDR1 comprising the amino acid sequence of SEQ ID NO:260; a CDR2 comprising the amino acid sequence of SEQ ID NO:261; and a CDR3 comprising the amino acid sequence of SEQ ID NO:262; and (iv) VL2 and VL4 comprise a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO: 6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 and VH4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:264; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:265; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:266; (ii) VH2 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (iii) VL1 and VL4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:260; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:261; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:262; and (iv) VL2 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57. In some aspects, (i) VH1 and VH4 comprise a CDR1 comprising the amino acid sequence of SEQ ID NO:264; a CDR2 comprising the amino acid sequence of SEQ ID NO:265; and a CDR3 comprising the amino acid sequence of SEQ ID NO:266; (ii) VH2 and VH3 comprise a CDR1 comprising the amino acid sequence of SEQ ID NO:59; a CDR2 comprising the amino acid sequence of SEQ ID NO:60; and a CDR3 comprising the amino acid sequence of SEQ ID NO:61; (iii) VL1 and VL4 comprise a CDR1 comprising the amino acid sequence of SEQ ID NO:260; a CDR2 comprising the amino acid sequence of SEQ ID NO:261; and a CDR3 comprising the amino acid sequence of SEQ ID NO:262; and (iv) VL2 and VL3 comprise a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 and VH4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:264; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:265; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:266; (ii) VH2 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iii) VL1 and VL4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:260; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:261; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:262; and (iv) VL2 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, (i) VH1 and VH4 comprise a CDR1 comprising the amino acid sequence of SEQ ID NO:264; a CDR2 comprising the amino acid sequence of SEQ ID NO:265; and a CDR3 comprising the amino acid sequence of SEQ ID NO:266; (ii) VH2 and VH3 comprise a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iii) VL1 and VL4 comprise a CDR1 comprising the amino acid sequence of SEQ ID NO:260; a CDR2 comprising the amino acid sequence of SEQ ID NO:261; and a CDR3 comprising the amino acid sequence of SEQ ID NO:262; and (iv) VL2 and VL3 comprise a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (ii) VH2 and VH4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:272; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:273; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:274; (iii) VL1 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; and (iv) VL2 and VL4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:268; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:269 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:270. In some aspects, (i) VH1 and VH3 comprise a CDR1 comprising the amino acid sequence of SEQ ID NO:59; a CDR2 comprising the amino acid sequence of SEQ ID NO:60; and a CDR3 comprising the amino acid sequence of SEQ ID NO:61; (ii) VH2 and VH4 comprise a CDR1 comprising the amino acid sequence of SEQ ID NO:272; a CDR2 comprising the amino acid sequence of SEQ ID NO:273; and a CDR3 comprising the amino acid sequence of SEQ ID NO:274; (iii) VL1 and VL3 comprise a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; and (iv) VL2 and VL4 comprise a CDR1 comprising the amino acid sequence of SEQ ID NO:268; a CDR2 comprising the amino acid sequence of SEQ ID NO:269 (having the sequence of GAS); and a CDR3 comprising the amino acid sequence of SEQ ID NO:270. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (ii) VH2 and VH4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:272; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:273; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:274; (iii) VL1 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278; and (iv) VL2 and VL4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:268; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:269 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:270. In some aspects, (i) VH1 and VH3 comprise a CDR1 comprising the amino acid sequence of SEQ ID NO:280; a CDR2 comprising the amino acid sequence of SEQ ID NO:281; and a CDR3 comprising the amino acid sequence of SEQ ID NO:282; (ii) VH2 and VH4 comprise a CDR1 comprising the amino acid sequence of SEQ ID NO:272; a CDR2 comprising the amino acid sequence of SEQ ID NO:273; and a CDR3 comprising the amino acid sequence of SEQ ID NO:274; (iii) VL1 and VL3 comprise a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278; and (iv) VL2 and VL4 comprise a CDR1 comprising the amino acid sequence of SEQ ID NO:268; a CDR2 comprising the amino acid sequence of SEQ ID NO:269 (having the sequence of GAS); and a CDR3 comprising the amino acid sequence of SEQ ID NO:270. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:272; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:273; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:274; (ii) VH2 and VH4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (iii) VL1 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:268; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:269 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:270; and (iv) VL2 and VL4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57. In some aspects, (i) VH1 and VH3 comprise a CDR1 comprising the amino acid sequence of SEQ ID NO:272; a CDR2 comprising the amino acid sequence of SEQ ID NO:273; and a CDR3 comprising the amino acid sequence of SEQ ID NO:274; (ii) VH2 and VH4 comprise a CDR1 comprising the amino acid sequence of SEQ ID NO:59; a CDR2 comprising the amino acid sequence of SEQ ID NO:60; and a CDR3 comprising the amino acid sequence of SEQ ID NO:61; (iii) VL1 and VL3 comprise a CDR1 comprising the amino acid sequence of SEQ ID NO:268; a CDR2 comprising the amino acid sequence of SEQ ID NO:269 (having the sequence of GAS); and a CDR3 comprising the amino acid sequence of SEQ ID NO:270; and (iv) VL2 and VL4 comprise a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:272; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:273; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:274; (ii) VH2 and VH4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iii) VL1 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:268; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:269 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:270; and (iv) VL2 and VL4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, (i) VH1 and VH3 comprise a CDR1 comprising the amino acid sequence of SEQ ID NO:272; a CDR2 comprising the amino acid sequence of SEQ ID NO:273; and a CDR3 comprising the amino acid sequence of SEQ ID NO:274; (ii) VH2 and VH4 comprise a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iii) VL1 and VL3 comprise a CDR1 comprising the amino acid sequence of SEQ ID NO:268; a CDR2 comprising the amino acid sequence of SEQ ID NO:269 (having the sequence of GAS); and a CDR3 comprising the amino acid sequence of SEQ ID NO:270; and (iv) VL2 and VL4 comprise a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 and VH4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:272; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:273; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:274; (ii) VH2 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (iii) VL1 and VL4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:268; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:269 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:270; and (iv) VL2 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57. In some aspects, (i) VH1 and VH4 comprise a CDR1 comprising the amino acid sequence of SEQ ID NO:272; a CDR2 comprising the amino acid sequence of SEQ ID NO:273; and a CDR3 comprising the amino acid sequence of SEQ ID NO:274; (ii) VH2 and VH3 comprise a CDR1 comprising the amino acid sequence of SEQ ID NO:59; a CDR2 comprising the amino acid sequence of SEQ ID NO:60; and a CDR3 comprising the amino acid sequence of SEQ ID NO:61; (iii) VL1 and VL4 comprise a CDR1 comprising the amino acid sequence of SEQ ID NO:268; a CDR2 comprising the amino acid sequence of SEQ ID NO:269 (having the sequence of GAS); and a CDR3 comprising the amino acid sequence of SEQ ID NO:270; and (iv) VL2 and VL3 comprise a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fe having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 and VH4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:272; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:273; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:274; (ii) VH2 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iii) VL1 and VL4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:268; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:269 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:270; and (iv) VL2 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, (i) VH1 and VH4 comprise a CDR1 comprising the amino acid sequence of SEQ ID NO:272; a CDR2 comprising the amino acid sequence of SEQ ID NO:273; and a CDR3 comprising the amino acid sequence of SEQ ID NO:274; (ii) VH2 and VH3 comprise a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iii) VL1 and VL4 comprise a CDR1 comprising the amino acid sequence of SEQ ID NO:268; a CDR2 comprising the amino acid sequence of SEQ ID NO:269 (having the sequence of GAS); and a CDR3 comprising the amino acid sequence of SEQ ID NO:270; and (iv) VL2 and VL3 comprise a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (ii) VH2 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:288; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:289; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:290; (iii) VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (iv) VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278; (v) VL2 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:284; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:285; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:286; and (vi) VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57. In some aspects, (i) VH1 comprises a CDR1 comprising the amino acid sequence of SEQ ID NO:280; a CDR2 comprising the amino acid sequence of SEQ ID NO:281; and a CDR3 comprising the amino acid sequence of SEQ ID NO:282; (ii) VH2 and VH3 comprise a CDR1 comprising the amino acid sequence of SEQ ID NO:288; a CDR2 comprising the amino acid sequence of SEQ ID NO:289; and a CDR3 comprising the amino acid sequence of SEQ ID NO:290; (iii) VH4 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; (iv) VL1 comprises a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278; (v) VL2 and VL3 comprise a CDR1 comprising the amino acid sequence of SEQ ID NO:284; a CDR2 comprising the amino acid sequence of SEQ ID NO:285; and a CDR3 comprising the amino acid sequence of SEQ ID NO:286; and (vi) VL4 comprises a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:288; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:289; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:290; (ii) VH2 and VH4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iii) VL1 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:284; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:285; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:286; and (iv) VL2 and VL4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, (i) VH1 and VH3 comprise a CDR1 comprising the amino acid sequence of SEQ ID NO:288; a CDR2 comprising the amino acid sequence of SEQ ID NO:289; and a CDR3 comprising the amino acid sequence of SEQ ID NO:290; (ii) VH2 and VH4 comprise a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iii) VL1 and VL3 comprise a CDR1 comprising the amino acid sequence of SEQ ID NO:284; a CDR2 comprising the amino acid sequence of SEQ ID NO:285; and a CDR3 comprising the amino acid sequence of SEQ ID NO:286; and (iv) VL2 and VL4 comprise a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (ii) VH2 and VH4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:288; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:289; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:290; (iii) VL1 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278; and (iv) VL2 and VL4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:284; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:285; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:286. In some aspects, (i) VH1 and VH3 comprise a CDR1 comprising the amino acid sequence of SEQ ID NO:280; a CDR2 comprising the amino acid sequence of SEQ ID NO:281; and a CDR3 comprising the amino acid sequence of SEQ ID NO:282; (ii) VH2 and VH4 comprise a CDR1 comprising the amino acid sequence of SEQ ID NO:288; a CDR2 comprising the amino acid sequence of SEQ ID NO:289; and a CDR3 comprising the amino acid sequence of SEQ ID NO:290; (iii) VL1 and VL3 comprise a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278; and (iv) VL2 and VL4 comprise a CDR1 comprising the amino acid sequence of SEQ ID NO:284; a CDR2 comprising the amino acid sequence of SEQ ID NO:285; and a CDR3 comprising the amino acid sequence of SEQ ID NO:286. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (ii) VH2 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:264; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:265; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:266; (iii) VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iv) VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; (v) VL2 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:260; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:261; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:262; and (vi) VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, (i) VH1 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; (ii) VH2 and VH3 comprise a CDR1 comprising the amino acid sequence of SEQ ID NO:264; a CDR2 comprising the amino acid sequence of SEQ ID NO:265; and a CDR3 comprising the amino acid sequence of SEQ ID NO:266; (iii) VH4 comprises a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iv) VL1 comprises a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; (v) VL2 and VL3 comprise a CDR1 comprising the amino acid sequence of SEQ ID NO:260; a CDR2 comprising the amino acid sequence of SEQ ID NO:261; and a CDR3 comprising the amino acid sequence of SEQ ID NO:262; and (vi) VL4 comprises a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1 or L4 and L7 or L10 comprise the amino acid sequence of SEQ ID NO:6; L2 or L5 and L8 or L11 comprise the amino acid sequence of SEQ ID NO: 27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (ii) VH2 and VH4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:264; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:265; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:266; (iii) VH3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iv) VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; (v) VL2 and VL4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:260; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:261; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:262; and (vi) VL3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, (i) VH1 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; (ii) VH2 and VH4 comprise a CDR1 comprising the amino acid sequence of SEQ ID NO:264; a CDR2 comprising the amino acid sequence of SEQ ID NO:265; and a CDR3 comprising the amino acid sequence of SEQ ID NO:266; (iii) VH3 comprises a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iv) VL1 comprises a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; (v) VL2 and VL4 comprise a CDR1 comprising the amino acid sequence of SEQ ID NO:260; a CDR2 comprising the amino acid sequence of SEQ ID NO:261; and a CDR3 comprising the amino acid sequence of SEQ ID NO:262; and (vi) VL3 comprises a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:264; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:265; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:266; (ii) VH2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (iii) VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iv) VL1 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:260; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:261; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:262; (v) VL2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; and (vi) VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, (i) VH1 and VH3 comprise a CDR1 comprising the amino acid sequence of SEQ ID NO:264; a CDR2 comprising the amino acid sequence of SEQ ID NO:265; and a CDR3 comprising the amino acid sequence of SEQ ID NO:266; (ii) VH2 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; (iii) VH4 comprises a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iv) VL1 and VL3 comprise a CDR1 comprising the amino acid sequence of SEQ ID NO:260; a CDR2 comprising the amino acid sequence of SEQ ID NO:261; and a CDR3 comprising the amino acid sequence of SEQ ID NO:262; (v) VL2 comprises a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; and (vi) VL4 comprises a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 and VH4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:264; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:265; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:266; (ii) VH2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (iii) VH3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iv) VL1 and VL4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:260; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:261; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:262; (v) VL2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; and (vi) VL3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, (i) VH1 and VH4 comprise a CDR1 comprising the amino acid sequence of SEQ ID NO:264; a CDR2 comprising the amino acid sequence of SEQ ID NO:265; and a CDR3 comprising the amino acid sequence of SEQ ID NO:266; (ii) VH2 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; (iii) VH3 comprises a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iv) VL1 and VL4 comprise a CDR1 comprising the amino acid sequence of SEQ ID NO:260; a CDR2 comprising the amino acid sequence of SEQ ID NO:261; and a CDR3 comprising the amino acid sequence of SEQ ID NO:262; (v) VL2 comprises a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; and (vi) VL3 comprises a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (ii) VH2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:264; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:265; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:266; (iii) VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iv) VL1 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; (v) VL2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:260; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:261; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:262; and (vi) VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, (i) VH1 and VH3 comprise a CDR1 comprising the amino acid sequence of SEQ ID NO:59; a CDR2 comprising the amino acid sequence of SEQ ID NO:60; and a CDR3 comprising the amino acid sequence of SEQ ID NO:61; (ii) VH2 comprises a CDR1 comprising the amino acid sequence of SEQ ID NO:264; a CDR2 comprising the amino acid sequence of SEQ ID NO:265; and a CDR3 comprising the amino acid sequence of SEQ ID NO:266; (iii) VH4 comprises a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iv) VL1 and VL3 comprise a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; (v) VL2 comprises a CDR1 comprising the amino acid sequence of SEQ ID NO:260; a CDR2 comprising the amino acid sequence of SEQ ID NO:261; and a CDR3 comprising the amino acid sequence of SEQ ID NO:262; and (vi) VL4 comprises a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 and VH4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (ii) VH2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:264; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:265; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:266; (iii) VH3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iv) VL1 and VL4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; (v) VL2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:260; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:261; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:262; and (vi) VL3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, (i) VH1 and VH4 comprise a CDR1 comprising the amino acid sequence of SEQ ID NO:59; a CDR2 comprising the amino acid sequence of SEQ ID NO:60; and a CDR3 comprising the amino acid sequence of SEQ ID NO:61; (ii) VH2 comprises a CDR1 comprising the amino acid sequence of SEQ ID NO:264; a CDR2 comprising the amino acid sequence of SEQ ID NO:265; and a CDR3 comprising the amino acid sequence of SEQ ID NO:266; (iii) VH3 comprises a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iv) VL1 and VL4 comprise a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; (v) VL2 comprises a CDR1 comprising the amino acid sequence of SEQ ID NO:260; a CDR2 comprising the amino acid sequence of SEQ ID NO:261; and a CDR3 comprising the amino acid sequence of SEQ ID NO:262; and (vi) VL3 comprises a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:264; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:265; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:266; (ii) VH2 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (iii) VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iv) VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:260; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:261; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:262; (v) VL2 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; and (vi) VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, (i) VH1 comprises a CDR1 comprising the amino acid sequence of SEQ ID NO:264; a CDR2 comprising the amino acid sequence of SEQ ID NO:265; and a CDR3 comprising the amino acid sequence of SEQ ID NO:266; (ii) VH2 and VH3 comprise a CDR1 comprising the amino acid sequence of SEQ ID NO:59; a CDR2 comprising the amino acid sequence of SEQ ID NO:60; and a CDR3 comprising the amino acid sequence of SEQ ID NO:61; (iii) VH4 comprises a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iv) VL1 comprises a CDR1 comprising the amino acid sequence of SEQ ID NO:260; a CDR2 comprising the amino acid sequence of SEQ ID NO:261; and a CDR3 comprising the amino acid sequence of SEQ ID NO:262; (v) VL2 and VL3 comprise a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; and (vi) VL4 comprises a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:264; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:265; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:266; (ii) VH2 and VH4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (iii) VH3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iv) VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:260; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:261; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:262; (v) VL2 and VL4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; and (vi) VL3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, (i) VH1 comprises a CDR1 comprising the amino acid sequence of SEQ ID NO:264; a CDR2 comprising the amino acid sequence of SEQ ID NO:265; and a CDR3 comprising the amino acid sequence of SEQ ID NO:266; (ii) VH2 and VH4 comprise a CDR1 comprising the amino acid sequence of SEQ ID NO:59; a CDR2 comprising the amino acid sequence of SEQ ID NO:60; and a CDR3 comprising the amino acid sequence of SEQ ID NO:61; (iii) VH3 comprises a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iv) VL1 comprises a CDR1 comprising the amino acid sequence of SEQ ID NO:260; a CDR2 comprising the amino acid sequence of SEQ ID NO:261; and a CDR3 comprising the amino acid sequence of SEQ ID NO:262; (v) VL2 and VL4 comprise a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; and (vi) VL3 comprises a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:264; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:265; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:266; (ii) VH2 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iii) VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (iv) VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:260; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:261; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:262; (v) VL2 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278; and (vi) VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57. In some aspects, (i) VH1 comprises a CDR1 comprising the amino acid sequence of SEQ ID NO:264; a CDR2 comprising the amino acid sequence of SEQ ID NO:265; and a CDR3 comprising the amino acid sequence of SEQ ID NO:266; (ii) VH2 and VH3 comprise a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iii) VH4 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; (iv) VL1 comprises a CDR1 comprising the amino acid sequence of SEQ ID NO:260; a CDR2 comprising the amino acid sequence of SEQ ID NO:261; and a CDR3 comprising the amino acid sequence of SEQ ID NO:262; (v) VL2 and VL3 comprise a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278; and (vi) VL4 comprises a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (ii) VH2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:264; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:265; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:266; (iii) VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (iv) VL1 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278; (v) VL2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:260; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:261; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:262; and (vi) VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57. In some aspects, (i) VH1 and VH3 comprise a CDR1 comprising the amino acid sequence of SEQ ID NO:280; a CDR2 comprising the amino acid sequence of SEQ ID NO:281; and a CDR3 comprising the amino acid sequence of SEQ ID NO:282; (ii) VH2 comprises a CDR1 comprising the amino acid sequence of SEQ ID NO:264; a CDR2 comprising the amino acid sequence of SEQ ID NO:265; and a CDR3 comprising the amino acid sequence of SEQ ID NO:266; (iii) VH4 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; (iv) VL1 and VL3 comprise a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278; (v) VL2 comprises a CDR1 comprising the amino acid sequence of SEQ ID NO:260; a CDR2 comprising the amino acid sequence of SEQ ID NO:261; and a CDR3 comprising the amino acid sequence of SEQ ID NO:262; and (vi) VL4 comprises a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (ii) VH2 and VH4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iii) VH3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:264; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:265; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:266; (iv) VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; (v) VL2 and VL4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278; and (vi) VL3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:260; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:261; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:262. In some aspects, (i) VH1 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; (ii) VH2 and VH4 comprise a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iii) VH3 comprises a CDR1 comprising the amino acid sequence of SEQ ID NO:264; a CDR2 comprising the amino acid sequence of SEQ ID NO:265; and a CDR3 comprising the amino acid sequence of SEQ ID NO:266; (iv) VL1 comprises a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; (v) VL2 and VL4 comprise a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278; and (vi) VL3 comprises a CDR1 comprising the amino acid sequence of SEQ ID NO:260; a CDR2 comprising the amino acid sequence of SEQ ID NO:261; and a CDR3 comprising the amino acid sequence of SEQ ID NO:262. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (ii) VH2 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iii) VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:264; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:265; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:266; (iv) VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; (v) VL2 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278; and (vi) VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:260; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:261; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:262. In some aspects, (i) VH1 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; (ii) VH2 and VH3 comprise a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iii) VH4 comprises a CDR1 comprising the amino acid sequence of SEQ ID NO:264; a CDR2 comprising the amino acid sequence of SEQ ID NO:265; and a CDR3 comprising the amino acid sequence of SEQ ID NO:266; (iv) VL1 comprises a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; (v) VL2 and VL3 comprise a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278; and (vi) VL4 comprises a CDR1 comprising the amino acid sequence of SEQ ID NO:260; a CDR2 comprising the amino acid sequence of SEQ ID NO:261; and a CDR3 comprising the amino acid sequence of SEQ ID NO:262. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (ii) VH2 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:272; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:273; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:274; (iii) VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iv) VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; (v) VL2 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:268; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:269 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:270; and (vi) VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, (i) VH1 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; (ii) VH2 and VH3 comprise a CDR1 comprising the amino acid sequence of SEQ ID NO:272; a CDR2 comprising the amino acid sequence of SEQ ID NO:273; and a CDR3 comprising the amino acid sequence of SEQ ID NO:274; (iii) VH4 comprises a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iv) VL1 comprises a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; (v) VL2 and VL3 comprise a CDR1 comprising the amino acid sequence of SEQ ID NO:268; a CDR2 comprising the amino acid sequence of SEQ ID NO:269 (having the sequence of GAS); and a CDR3 comprising the amino acid sequence of SEQ ID NO:270; and (vi) VL4 comprises a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (ii) VH2 and VH4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:272; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:273; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:274; (iii) VH3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iv) VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; (v) VL2 and VL4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:268; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:269 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:270; and (vi) VL3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, (i) VH1 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; (ii) VH2 and VH4 comprise a CDR1 comprising the amino acid sequence of SEQ ID NO:272; a CDR2 comprising the amino acid sequence of SEQ ID NO:273; and a CDR3 comprising the amino acid sequence of SEQ ID NO:274; (iii) VH3 comprises a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iv) VL1 comprises a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; (v) VL2 and VL4 comprise a CDR1 comprising the amino acid sequence of SEQ ID NO:268; a CDR2 comprising the amino acid sequence of SEQ ID NO:269 (having the sequence of GAS); and a CDR3 comprising the amino acid sequence of SEQ ID NO:270; and (vi) VL3 comprises a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO: 27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:272; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:273; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:274; (ii) VH2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (iii) VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iv) VL1 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:268; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:269 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:270; (v) VL2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; and (vi) VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, (i) VH1 and VH3 comprise a CDR1 comprising the amino acid sequence of SEQ ID NO:272; a CDR2 comprising the amino acid sequence of SEQ ID NO:273; and a CDR3 comprising the amino acid sequence of SEQ ID NO:274; (ii) VH2 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; (iii) VH4 comprises a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iv) VL1 and VL3 comprise a CDR1 comprising the amino acid sequence of SEQ ID NO:268; a CDR2 comprising the amino acid sequence of SEQ ID NO:269 (having the sequence of GAS); and a CDR3 comprising the amino acid sequence of SEQ ID NO:270; (v) VL2 comprises a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; and (vi) VL4 comprises a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 and VH4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:272; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:273; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:274; (ii) VH2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (iii) VH3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iv) VL1 and VL4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:268; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:269 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:270; (v) VL2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; and (vi) VL3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, (i) VH1 and VH4 comprise a CDR1 comprising the amino acid sequence of SEQ ID NO:272; a CDR2 comprising the amino acid sequence of SEQ ID NO:273; and a CDR3 comprising the amino acid sequence of SEQ ID NO:274; (ii) VH2 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; (iii) VH3 comprises a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iv) VL1 and VL4 comprise a CDR1 comprising the amino acid sequence of SEQ ID NO:268; a CDR2 comprising the amino acid sequence of SEQ ID NO:269 (having the sequence of GAS); and a CDR3 comprising the amino acid sequence of SEQ ID NO:270; (v) VL2 comprises a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; and (vi) VL3 comprises a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (ii) VH2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:272; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:273; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:274; (iii) VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iv) VL1 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; (v) VL2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:268; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:269 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:270; and (vi) VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, (i) VH1 and VH3 comprise a CDR1 comprising the amino acid sequence of SEQ ID NO:59; a CDR2 comprising the amino acid sequence of SEQ ID NO:60; and a CDR3 comprising the amino acid sequence of SEQ ID NO:61; (ii) VH2 comprises a CDR1 comprising the amino acid sequence of SEQ ID NO:272; a CDR2 comprising the amino acid sequence of SEQ ID NO:273 and a CDR3 comprising the amino acid sequence of SEQ ID NO:274; (iii) VH4 comprises a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iv) VL1 and VL3 comprise a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; (v) VL2 comprises a CDR1 comprising the amino acid sequence of SEQ ID NO:268; a CDR2 comprising the amino acid sequence of SEQ ID NO:269 (having the sequence of GAS); and a CDR3 comprising the amino acid sequence of SEQ ID NO:270; and (vi) VL4 comprises a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO: 27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 and VH4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (ii) VH2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:272; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:273; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:274; (iii) VH3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iv) VL1 and VL4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; (v) VL2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:268; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:269 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:270; and (vi) VL3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, (i) VH1 and VH4 comprise a CDR1 comprising the amino acid sequence of SEQ ID NO:59; a CDR2 comprising the amino acid sequence of SEQ ID NO:60; and a CDR3 comprising the amino acid sequence of SEQ ID NO:61; (ii) VH2 comprises a CDR1 comprising the amino acid sequence of SEQ ID NO:272; a CDR2 comprising the amino acid sequence of SEQ ID NO:273 and a CDR3 comprising the amino acid sequence of SEQ ID NO:274; (iii) VH3 comprises a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iv) VL1 and VL4 comprise a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; (v) VL2 comprises a CDR1 comprising the amino acid sequence of SEQ ID NO:268; a CDR2 comprising the amino acid sequence of SEQ ID NO:269 (having the sequence of GAS); and a CDR3 comprising the amino acid sequence of SEQ ID NO:270; and (vi) VL3 comprises a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:272; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:273; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:274; (ii) VH2 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (iii) VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iv) VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:268; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:269 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:270; (v) VL2 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; and (vi) VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, (i) VH1 comprises a CDR1 comprising the amino acid sequence of SEQ ID NO:272; a CDR2 comprising the amino acid sequence of SEQ ID NO:273; and a CDR3 comprising the amino acid sequence of SEQ ID NO:274; (ii) VH2 and VH3 comprise a CDR1 comprising the amino acid sequence of SEQ ID NO:59; a CDR2 comprising the amino acid sequence of SEQ ID NO:60; and a CDR3 comprising the amino acid sequence of SEQ ID NO:61; (iii) VH4 comprises a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iv) VL1 comprises a CDR1 comprising the amino acid sequence of SEQ ID NO:268; a CDR2 comprising the amino acid sequence of SEQ ID NO:269 (having the sequence of GAS); and a CDR3 comprising the amino acid sequence of SEQ ID NO:270; (v) VL2 and VL3 comprise a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; and (vi) VL4 comprises a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO: 27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:272; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:273; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:274; (ii) VH2 and VH4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (iii) VH3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iv) VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:268; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:269 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:270; (v) VL2 and VL4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; and (vi) VL3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, (i) VH1 comprises a CDR1 comprising the amino acid sequence of SEQ ID NO:272; a CDR2 comprising the amino acid sequence of SEQ ID NO:273; and a CDR3 comprising the amino acid sequence of SEQ ID NO:274; (ii) VH2 and VH4 comprise a CDR1 comprising the amino acid sequence of SEQ ID NO:59; a CDR2 comprising the amino acid sequence of SEQ ID NO:60; and a CDR3 comprising the amino acid sequence of SEQ ID NO:61; (iii) VH3 comprises a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iv) VL1 comprises a CDR1 comprising the amino acid sequence of SEQ ID NO:268; a CDR2 comprising the amino acid sequence of SEQ ID NO:269 (having the sequence of GAS); and a CDR3 comprising the amino acid sequence of SEQ ID NO:270; (v) VL2 and VL4 comprise a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; and (vi) VL3 comprises a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO: 27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:272; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:273; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:274; (ii) VH2 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iii) VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (iv) VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:268; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:269 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:270; (v) VL2 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278; and (vi) VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57. In some aspects, (i) VH1 comprises a CDR1 comprising the amino acid sequence of SEQ ID NO:272; a CDR2 comprising the amino acid sequence of SEQ ID NO:273; and a CDR3 comprising the amino acid sequence of SEQ ID NO:274; (ii) VH2 and VH3 comprise a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iii) VH4 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; (iv) VL1 comprises a CDR1 comprising the amino acid sequence of SEQ ID NO:268; a CDR2 comprising the amino acid sequence of SEQ ID NO:269 (having the sequence of GAS); and a CDR3 comprising the amino acid sequence of SEQ ID NO:270; (v) VL2 and VL3 comprise a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278; and (vi) VL4 comprises a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:272; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:273; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:274; (ii) VH1 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iii) VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (iv) VL2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:268; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:269 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:270; (v) VL1 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278; and (vi) VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57. In some aspects, (i) VH2 comprises a CDR1 comprising the amino acid sequence of SEQ ID NO:272; a CDR2 comprising the amino acid sequence of SEQ ID NO:273; and a CDR3 comprising the amino acid sequence of SEQ ID NO:274; (ii) VH1 and VH3 comprise a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iii) VH4 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; (iv) VL2 comprises a CDR1 comprising the amino acid sequence of SEQ ID NO:268; a CDR2 comprising the amino acid sequence of SEQ ID NO:269 (having the sequence of GAS); and a CDR3 comprising the amino acid sequence of SEQ ID NO:270; (v) VL1 and VL3 comprise a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278; and (vi) VL4 comprises a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57. In some aspects, L1-L16 comprise the amino acid sequence of any one of SEQ ID NOs:1-34. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fe having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (ii) VH2 and VH4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iii) VH3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:272; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:273; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:274; (iv) VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; (v) VL2 and VL4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278; and (vi) VL3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:268; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:269 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:270. In some aspects, (i) VH1 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; (ii) VH2 and VH4 comprise a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iii) VH3 comprises a CDR1 comprising the amino acid sequence of SEQ ID NO:272; a CDR2 comprising the amino acid sequence of SEQ ID NO:273; and a CDR3 comprising the amino acid sequence of SEQ ID NO:274; (iv) VL1 comprises a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; (v) VL2 and VL4 comprise a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278; and (vi) VL3 comprises a CDR1 comprising the amino acid sequence of SEQ ID NO:268; a CDR2 comprising the amino acid sequence of SEQ ID NO:269 (having the sequence of GAS); and a CDR3 comprising the amino acid sequence of SEQ ID NO:270. In some aspects, L1-L16 comprise the amino acid sequence of any one of, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (ii) VH2 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iii) VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:272; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:273; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:274; (iv) VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; (v) VL2 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278; and (vi) VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:268; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:269 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:270. In some aspects, (i) VH1 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; (ii) VH2 and VH3 comprise a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iii) VH4 comprises a CDR1 comprising the amino acid sequence of SEQ ID NO:272; a CDR2 comprising the amino acid sequence of SEQ ID NO:273; and a CDR3 comprising the amino acid sequence of SEQ ID NO:274; (iv) VL1 comprises a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; (v) VL2 and VL3 comprise a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278; and (vi) VL4 comprises a CDR1 comprising the amino acid sequence of SEQ ID NO:268; a CDR2 comprising the amino acid sequence of SEQ ID NO:269 (having the sequence of GAS); and a CDR3 comprising the amino acid sequence of SEQ ID NO:270. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (ii) VH2 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:288; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:289; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:290; (iii) VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iv) VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; (v) VL2 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:284; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:285; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:286; and (vi) VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, (i) VH1 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; (ii) VH2 and VH3 comprise a CDR1 comprising the amino acid sequence of SEQ ID NO:288; a CDR2 comprising the amino acid sequence of SEQ ID NO:289; and a CDR3 comprising the amino acid sequence of SEQ ID NO:290; (iii) VH4 comprises a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iv) VL1 comprises a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; (v) VL2 and VL3 comprise a CDR1 comprising the amino acid sequence of SEQ ID NO:284; a CDR2 comprising the amino acid sequence of SEQ ID NO:285; and a CDR3 comprising the amino acid sequence of SEQ ID NO:286; and (vi) VL4 comprises a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (ii) VH2 and VH4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:288; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:289; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:290; (iii) VH3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iv) VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; (v) VL2 and VL4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:284; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:285; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:286; and (vi) VL3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, (i) VH1 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; (ii) VH2 and VH4 comprise a CDR1 comprising the amino acid sequence of SEQ ID NO:288; a CDR2 comprising the amino acid sequence of SEQ ID NO:289; and a CDR3 comprising the amino acid sequence of SEQ ID NO:290; (iii) VH3 comprises a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iv) VL1 comprises a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; (v) VL2 and VL4 comprise a CDR1 comprising the amino acid sequence of SEQ ID NO:284; a CDR2 comprising the amino acid sequence of SEQ ID NO:285; and a CDR3 comprising the amino acid sequence of SEQ ID NO:286; and (vi) VL3 comprises a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:288; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:289; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:290; (ii) VH2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (iii) VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iv) VL1 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:284; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:285; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:286; (v) VL2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; and (vi) VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, (i) VH1 and VH3 comprise a CDR1 comprising the amino acid sequence of SEQ ID NO:288; a CDR2 comprising the amino acid sequence of SEQ ID NO:289; and a CDR3 comprising the amino acid sequence of SEQ ID NO:290; (ii) VH2 comprises a CDR1 comprising the amino acid sequence of SEQ ID NO:59; a CDR2 comprising the amino acid sequence of SEQ ID NO:60 and a CDR3 comprising the amino acid sequence of SEQ ID NO:61; (iii) VH4 comprises a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iv) VL1 and VL3 comprise a CDR1 comprising the amino acid sequence of SEQ ID NO:284; a CDR2 comprising the amino acid sequence of SEQ ID NO:285; and a CDR3 comprising the amino acid sequence of SEQ ID NO:286; (v) VL2 comprises a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; and (vi) VL4 comprises a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 and VH4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:288; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:289; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:290; (ii) VH2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (iii) VH3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iv) VL1 and VL4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:284; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:285; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:286; (v) VL2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; and (vi) VL3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, (i) VH1 and VH4 comprise a CDR1 comprising the amino acid sequence of SEQ ID NO:288; a CDR2 comprising the amino acid sequence of SEQ ID NO:289; and a CDR3 comprising the amino acid sequence of SEQ ID NO:290; (ii) VH2 comprises a CDR1 comprising the amino acid sequence of SEQ ID NO:59; a CDR2 comprising the amino acid sequence of SEQ ID NO:60 and a CDR3 comprising the amino acid sequence of SEQ ID NO:61; (iii) VH3 comprises a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iv) VL1 and VL4 comprise a CDR1 comprising the amino acid sequence of SEQ ID NO:284; a CDR2 comprising the amino acid sequence of SEQ ID NO:285; and a CDR3 comprising the amino acid sequence of SEQ ID NO:286; (v) VL2 comprises a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; and (vi) VL3 comprises a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (ii) VH2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:288; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:289; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:290; (iii) VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iv) VL1 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; (v) VL2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:284; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:285; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:286; and (vi) VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, (i) VH1 and VH3 comprise a CDR1 comprising the amino acid sequence of SEQ ID NO:59; a CDR2 comprising the amino acid sequence of SEQ ID NO:60; and a CDR3 comprising the amino acid sequence of SEQ ID NO:61; (ii) VH2 comprises a CDR1 comprising the amino acid sequence of SEQ ID NO:288; a CDR2 comprising the amino acid sequence of SEQ ID NO:289; and a CDR3 comprising the amino acid sequence of SEQ ID NO:290; (iii) VH4 comprises a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iv) VL1 and VL3 comprise a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; (v) VL2 comprises a CDR1 comprising the amino acid sequence of SEQ ID NO:284; a CDR2 comprising the amino acid sequence of SEQ ID NO:285; and a CDR3 comprising the amino acid sequence of SEQ ID NO:286; and (vi) VL4 comprises a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 and VH4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (ii) VH2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:288; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:289; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:290; (iii) VH3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iv) VL1 and VL4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; (v) VL2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:284; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:285; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:286; and (vi) VL3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, (i) VH1 and VH4 comprise a CDR1 comprising the amino acid sequence of SEQ ID NO:59; a CDR2 comprising the amino acid sequence of SEQ ID NO:60; and a CDR3 comprising the amino acid sequence of SEQ ID NO:61; (ii) VH2 comprises a CDR1 comprising the amino acid sequence of SEQ ID NO:288; a CDR2 comprising the amino acid sequence of SEQ ID NO:289; and a CDR3 comprising the amino acid sequence of SEQ ID NO:290; (iii) VH3 comprises a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iv) VL1 and VL4 comprise a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; (v) VL2 comprises a CDR1 comprising the amino acid sequence of SEQ ID NO:284; a CDR2 comprising the amino acid sequence of SEQ ID NO:285; and a CDR3 comprising the amino acid sequence of SEQ ID NO:286; and (vi) VL3 comprises a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH2 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (ii) VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:288; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:289; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:290; (iii) VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iv) VL2 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; (v) VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:284; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:285; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:286; and (vi) VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, (i) VH2 and VH3 comprise a CDR1 comprising the amino acid sequence of SEQ ID NO:59; a CDR2 comprising the amino acid sequence of SEQ ID NO:60; and a CDR3 comprising the amino acid sequence of SEQ ID NO:61; (ii) VH1 comprises a CDR1 comprising the amino acid sequence of SEQ ID NO:288; a CDR2 comprising the amino acid sequence of SEQ ID NO:289; and a CDR3 comprising the amino acid sequence of SEQ ID NO:290; (iii) VH4 comprises a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iv) VL2 and VL3 comprise a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; (v) VL1 comprises a CDR1 comprising the amino acid sequence of SEQ ID NO:284; a CDR2 comprising the amino acid sequence of SEQ ID NO:285; and a CDR3 comprising the amino acid sequence of SEQ ID NO:286; and (vi) VL4 comprises a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fe having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH2 and VH4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (ii) VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:288; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:289; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:290; (iii) VH3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iv) VL2 and VL4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; (v) VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:284; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:285; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:286; and (vi) VL3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, (i) VH2 and VH4 comprise a CDR1 comprising the amino acid sequence of SEQ ID NO:59; a CDR2 comprising the amino acid sequence of SEQ ID NO:60; and a CDR3 comprising the amino acid sequence of SEQ ID NO:61; (ii) VH1 comprises a CDR1 comprising the amino acid sequence of SEQ ID NO:288; a CDR2 comprising the amino acid sequence of SEQ ID NO:289; and a CDR3 comprising the amino acid sequence of SEQ ID NO:290; (iii) VH3 comprises a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iv) VL2 and VL4 comprise a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; (v) VL1 comprises a CDR1 comprising the amino acid sequence of SEQ ID NO:284; a CDR2 comprising the amino acid sequence of SEQ ID NO:285; and a CDR3 comprising the amino acid sequence of SEQ ID NO:286; and (vi) VL3 comprises a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:288; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:289; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:290; (ii) VH2 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iii) VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (iv) VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:284; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:285; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:286; (v) VL2 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278; and (vi) VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57. In some aspects, (i) VH1 comprises a CDR1 comprising the amino acid sequence of SEQ ID NO:288; a CDR2 comprising the amino acid sequence of SEQ ID NO:289; and a CDR3 comprising the amino acid sequence of SEQ ID NO:290; (ii) VH2 and VH3 comprise a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iii) VH4 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; (iv) VL1 comprises a CDR1 comprising the amino acid sequence of SEQ ID NO:284; a CDR2 comprising the amino acid sequence of SEQ ID NO:285; and a CDR3 comprising the amino acid sequence of SEQ ID NO:286; (v) VL2 and VL3 comprise a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278; and (vi) VL4 comprises a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:288; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:289; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:290; (ii) VH1 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iii) VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (iv) VL2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:284; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:285; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:286; (v) VL1 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278; and (vi) VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57. In some aspects, (i) VH2 comprises a CDR1 comprising the amino acid sequence of SEQ ID NO:288; a CDR2 comprising the amino acid sequence of SEQ ID NO:289; and a CDR3 comprising the amino acid sequence of SEQ ID NO:290; (ii) VH1 and VH3 comprise a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iii) VH4 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; (iv) VL2 comprises a CDR1 comprising the amino acid sequence of SEQ ID NO:284; a CDR2 comprising the amino acid sequence of SEQ ID NO:285; and a CDR3 comprising the amino acid sequence of SEQ ID NO:286; (v) VL1 and VL3 comprise a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278; and (vi) VL4 comprises a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (ii) VH2 and VH4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iii) VH3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:288; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:289; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:290; (iv) VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; (v) VL2 and VL4 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278; and (vi) VL3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:284; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:285; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:286. In some aspects, (i) VH1 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; (ii) VH2 and VH4 comprise a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iii) VH3 comprises a CDR1 comprising the amino acid sequence of SEQ ID NO:288; a CDR2 comprising the amino acid sequence of SEQ ID NO:289; and a CDR3 comprising the amino acid sequence of SEQ ID NO:290; (iv) VL1 comprises a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; (v) VL2 and VL4 comprise a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278; and (vi) VL3 comprises a CDR1 comprising the amino acid sequence of SEQ ID NO:284; a CDR2 comprising the amino acid sequence of SEQ ID NO:285; and a CDR3 comprising the amino acid sequence of SEQ ID NO:286. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (ii) VH2 and VH3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iii) VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:288; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:289; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:290; (iv) VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; (v) VL2 and VL3 comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278; and (vi) VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:284; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:285; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:286. In some aspects, (i) VH1 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; (ii) VH2 and VH3 comprise a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iii) VH4 comprises a CDR1 comprising the amino acid sequence of SEQ ID NO:288; a CDR2 comprising the amino acid sequence of SEQ ID NO:289; and a CDR3 comprising the amino acid sequence of SEQ ID NO:290; (iv) VL1 comprises a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; (v) VL2 and VL3 comprise a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278; and (vi) VL4 comprises a CDR1 comprising the amino acid sequence of SEQ ID NO:284; a CDR2 comprising the amino acid sequence of SEQ ID NO:285; and a CDR3 comprising the amino acid sequence of SEQ ID NO:286. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (ii) VH2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:272; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:273; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:274; (iii) VH3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:264; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:265; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:266; (iv) VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (v) VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; (vi) VL2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:268; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:269 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:270; (vii) VL3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:260; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:261; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:262; and (viii) VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, (i) VH1 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; (ii) VH2 comprises a CDR1 comprising the amino acid sequence of SEQ ID NO:272; a CDR2 comprising the amino acid sequence of SEQ ID NO:273; and a CDR3 comprising the amino acid sequence of SEQ ID NO:274; (iii) VH3 comprises a CDR1 comprising the amino acid sequence of SEQ ID NO:264; a CDR2 comprising the amino acid sequence of SEQ ID NO:265; and a CDR3 comprising the amino acid sequence of SEQ ID NO:266; (iv) VH4 comprises a CDR1 comprising the amino acid sequence of SEQ ID NO:280; a CDR2 comprising the amino acid sequence of SEQ ID NO:281; and a CDR3 comprising the amino acid sequence of SEQ ID NO:282; (v) VL1 comprises a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; (vi) VL2 comprises a CDR1 comprising the amino acid sequence of SEQ ID NO:268; a CDR2 comprising the amino acid sequence of SEQ ID NO:269 (having the sequence of GAS); and a CDR3 comprising the amino acid sequence of SEQ ID NO:270; (vii) VL3 comprises a CDR1 comprising the amino acid sequence of SEQ ID NO:260; a CDR2 comprising the amino acid sequence of SEQ ID NO:261; and a CDR3 comprising the amino acid sequence of SEQ ID NO:262; and (viii) VL4 comprises a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (ii) VH2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:288; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:289; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:290; (iii) VH3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iv) VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:264; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:265; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:266; (v) VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; (vi) VL2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:284; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:285; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:286; (vii) VL3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278; and (viii) VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:260; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:261; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:262. In some aspects, (i) VH1 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; (ii) VH2 comprises a CDR1 comprising the amino acid sequence of SEQ ID NO:288; a CDR2 comprising the amino acid sequence of SEQ ID NO:289; and a CDR3 comprising the amino acid sequence of SEQ ID NO:290; (iii) VH3 comprises a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iv) VH4 comprises a CDR1 comprising the amino acid sequence of SEQ ID NO:264; a CDR2 comprising the amino acid sequence of SEQ ID NO:265; and a CDR3 comprising the amino acid sequence of SEQ ID NO:266; (v) VL1 comprises a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; (vi) VL2 comprises a CDR1 comprising the amino acid sequence of SEQ ID NO:284; a CDR2 comprising the amino acid sequence of SEQ ID NO:285; and a CDR3 comprising the amino acid sequence of SEQ ID NO:286; (vii) VL3 comprises a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278; and (viii) VL4 comprises a CDR1 comprising the amino acid sequence of SEQ ID NO:260; a CDR2 comprising the amino acid sequence of SEQ ID NO:261; and a CDR3 comprising the amino acid sequence of SEQ ID NO:262. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (ii) VH2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:264; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:265; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:266; (iii) VH3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:272; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:273; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:274; (iv) VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (v) VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; (vi) VL2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:260; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:261; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:262; (vii) VL3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:268; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:269 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:270; and (viii) VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278. In some aspects, (i) VH1 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; (ii) VH2 comprises a CDR1 comprising the amino acid sequence of SEQ ID NO:264; a CDR2 comprising the amino acid sequence of SEQ ID NO:265; and a CDR3 comprising the amino acid sequence of SEQ ID NO:266; (iii) VH3 comprises a CDR1 comprising the amino acid sequence of SEQ ID NO:272; a CDR2 comprising the amino acid sequence of SEQ ID NO:273; and a CDR3 comprising the amino acid sequence of SEQ ID NO:274; (iv) VH4 comprises a CDR1 comprising the amino acid sequence of SEQ ID NO:280; a CDR2 comprising the amino acid sequence of SEQ ID NO:281; and a CDR3 comprising the amino acid sequence of SEQ ID NO:282; (v) VL1 comprises a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; (vi) VL2 comprises a CDR1 comprising the amino acid sequence of SEQ ID NO:260; a CDR2 comprising the amino acid sequence of SEQ ID NO:261; and a CDR3 comprising the amino acid sequence of SEQ ID NO:262; (vii) VL3 comprises a CDR1 comprising the amino acid sequence of SEQ ID NO:268; a CDR2 comprising the amino acid sequence of SEQ ID NO:269 (having the sequence of GAS); and a CDR3 comprising the amino acid sequence of SEQ ID NO:270; and (viii) VL4 comprises a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (ii) VH2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:264; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:265; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:266; (iii) VH3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iv) VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:288; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:289; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:290; (v) VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; (vi) VL2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:260; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:261; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:262; (vii) VL3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278; and (viii) VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:284; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:285; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:286. In some aspects, (i) VH1 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; (ii) VH2 comprises a CDR1 comprising the amino acid sequence of SEQ ID NO:264; a CDR2 comprising the amino acid sequence of SEQ ID NO:265; and a CDR3 comprising the amino acid sequence of SEQ ID NO:266; (iii) VH3 comprises a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iv) VH4 comprises a CDR1 comprising the amino acid sequence of SEQ ID NO:288; a CDR2 comprising the amino acid sequence of SEQ ID NO:289; and a CDR3 comprising the amino acid sequence of SEQ ID NO:290; (v) VL1 comprises a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; (vi) VL2 comprises a CDR1 comprising the amino acid sequence of SEQ ID NO:260; a CDR2 comprising the amino acid sequence of SEQ ID NO:261; and a CDR3 comprising the amino acid sequence of SEQ ID NO:262; (vii) VL3 comprises a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278; and (viii) VL4 comprises a CDR1 comprising the amino acid sequence of SEQ ID NO:284; a CDR2 comprising the amino acid sequence of SEQ ID NO:285; and a CDR3 comprising the amino acid sequence of SEQ ID NO:286. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, (i) VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:59; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:60; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:61; (ii) VH2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:272; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:273; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:274; (iii) VH3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; (iv) VH4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:288; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:289; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:290; (v) VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:55; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:57; (vi) VL2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:268; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:269 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:270; (vii) VL3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277 (having the sequence of LGS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278; and (viii) VL4 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:284; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:285; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:286. In some aspects, (i) VH1 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61; (ii) VH2 comprises a CDR1 comprising the amino acid sequence of SEQ ID NO:272; a CDR2 comprising the amino acid sequence of SEQ ID NO:273; and a CDR3 comprising the amino acid sequence of SEQ ID NO:274; (iii) VH3 comprises a CDR1 comprising the amino acid sequence of SEQ ID NO:280; a CDR2 comprising the amino acid sequence of SEQ ID NO:281; and a CDR3 comprising the amino acid sequence of SEQ ID NO:282; (iv) VH4 comprises a CDR1 comprising the amino acid sequence of SEQ ID NO:288; a CDR2 comprising the amino acid sequence of SEQ ID NO:289; and a CDR3 comprising the amino acid sequence of SEQ ID NO:290; (v) VL1 comprises a CDR1 comprising the amino acid sequence of SEQ ID NO:55; a CDR2 comprising the amino acid sequence of SEQ ID NO:56 (having the sequence of GTN); and a CDR3 comprising the amino acid sequence of SEQ ID NO:57; (vi) VL2 comprises a CDR1 comprising the amino acid sequence of SEQ ID NO:268; a CDR2 comprising the amino acid sequence of SEQ ID NO:269 (having the sequence of GAS); and a CDR3 comprising the amino acid sequence of SEQ ID NO:270; (vii) VL3 comprises a CDR1 comprising the amino acid sequence of SEQ ID NO:276; a CDR2 comprising the amino acid sequence of SEQ ID NO:277 (having the sequence of LGS); and a CDR3 comprising the amino acid sequence of SEQ ID NO:278; and (viii) VL4 comprises a CDR1 comprising the amino acid sequence of SEQ ID NO:284; a CDR2 comprising the amino acid sequence of SEQ ID NO:285; and a CDR3 comprising the amino acid sequence of SEQ ID NO:286. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO: 27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, CDR1, CDR2 and CDR3 of each of VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, CDR1, CDR2 and CDR3 of each of VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, provided herein is an antigen binding polypeptide complex comprising a first polypeptide and a second polypeptide; wherein the first polypeptide has a structure represented by VL1-L1-VH1 or VH1-L2-VL1; wherein the second polypeptide has a structure represented by VL2-L3-VH2 or VH2-L4-VL2; wherein VL1 is a first immunoglobulin light chain variable region that specifically binds to a SARS-CoV-2 protein; VH1 is a first immunoglobulin heavy chain variable region that specifically binds to a SARS-CoV-2 protein; wherein VL2 is a second immunoglobulin light chain variable region that specifically binds to a SARS-CoV-2 protein; wherein VH2 is a second immunoglobulin heavy chain variable region that specifically binds to a SARS-CoV-2 protein; and L1-L4 are amino acid linkers. In some aspects, provided herein is an antigen binding polypeptide complex comprising a first polypeptide and a second polypeptide; wherein the first polypeptide has a structure represented by VL1-L1-VH1-L2-Fc or VH1-L3-VL1-L4-Fc; wherein the second polypeptide has a structure represented by VL2-L5-VH2-L6-Fc or VH2-L7-VL2-L8-Fc; wherein VL1 is a first immunoglobulin light chain variable region that specifically binds to a SARS-CoV-2 protein; VH1 is a first immunoglobulin heavy chain variable region that specifically binds to a SARS-CoV-2 protein; wherein VL2 is a second immunoglobulin light chain variable region that specifically binds to a SARS-CoV-2 protein; wherein VH2 is a second immunoglobulin heavy chain variable region that specifically binds to a SARS-CoV-2 protein; L1-L8 are amino acid linkers; and Fc is a region comprising an immunoglobulin heavy chain constant region 2 (CH2), an immunoglobulin heavy chain constant region 3 (CH3), and optionally, an immunoglobulin hinge. In some aspects, provided herein is an antigen binding polypeptide complex comprising a first polypeptide and a second polypeptide; wherein the first polypeptide has a structure represented by VL1-L1-VH1; the second polypeptide has a structure represented by VL2-L3-VH2 or VH2-L4-VL2; and wherein the first and/or second polypeptide may have an Fc as shown above. In some aspects, provided herein is an antigen binding polypeptide complex comprising a first polypeptide and a second polypeptide; wherein the first polypeptide has a structure represented by VH1-L2-VL1; the second polypeptide has a structure represented by VL2-L3-VH2 or VH2-L4-VL2; and wherein the first and/or second polypeptide may have an Fc as shown above.

In some aspects, VH1 and VH2 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:263; and VL1 and VL2 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:259. In some aspects, VH1 and VH2 comprise the amino acid sequence of SEQ ID NO:263; and VL1 and VL2 comprise the amino acid sequence of SEQ ID NO:259. In some aspects, L1 or L2 and L3 or L4 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L2 and L3 or L4 comprise the amino acid sequence of SEQ ID NO:27. In some aspects, L1 or L3 and L5 or L7 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L3 and L5 or L7 comprise the amino acid sequence of SEQ ID NO:27. In some aspects, L2 or L4 and L6 or L8 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L2 or L4 and L6 or L8 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some preferred aspects, VH1, VH2, VL1 and VL2 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, VH1, VH2, VL1 and VL2 comprise or consist of the corresponding amino acid sequences specified above, optionally with (i) an Fc comprising or consisting of the corresponding amino acid sequence recited above; and/or (ii) one or more linker sequence as specified above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:329; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:329. In some aspects, both the first polypeptide and the second polypeptide have at least 90% identity to SEQ ID NO:329. In some aspects, the first polypeptide comprises the amino acid sequence of SEQ ID NO:329; and the second polypeptide comprises the amino acid sequence of SEQ ID NO:329. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:330; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:330. In some aspects, both the first polypeptide and the second polypeptide comprise an amino acid sequence encoded by a polynucleotide having at least 90% identity to SEQ ID NO:330. In some aspects, the first polypeptide comprises an amino acid sequence encoded by the polynucleotide of SEQ ID NO:330; and the second polypeptide comprises an amino acid sequence encoded by the polynucleotide of SEQ ID NO:330.

In some aspects, VH1 and VH2 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:271; and VL1 and VL2 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:267. In some aspects, VH1 and VH2 comprise the amino acid sequence of SEQ ID NO:271; and VL1 and VL2 comprise the amino acid sequence of SEQ ID NO:267. In some aspects, L1 or L2 and L3 or L4 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L2 and L3 or L4 comprise the amino acid sequence of SEQ ID NO:27. In some aspects, L1 or L3 and L5 or L7 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L3 and L5 or L7 comprise the amino acid sequence of SEQ ID NO:27. In some aspects, L2 or L4 and L6 or L8 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L2 or L4 and L6 or L8 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some preferred aspects, VH1, VH2, VL1 and VL2 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, VH1, VH2, VL1 and VL2 comprise or consist of the corresponding amino acid sequences specified above, optionally with (i) an Fc comprising or consisting of the corresponding amino acid sequence recited above; and/or (ii) one or more linker sequence as specified above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:331; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:331. In some aspects, both the first polypeptide and the second polypeptide have at least 90% identity to SEQ ID NO:331. In some aspects, the first polypeptide comprises the amino acid sequence of SEQ ID NO:331; and the second polypeptide comprises the amino acid sequence of SEQ ID NO:331. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:332; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:332. In some aspects, both the first polypeptide and the second polypeptide comprise an amino acid sequence encoded by a polynucleotide having at least 90% identity to SEQ ID NO:332. In some aspects, the first polypeptide comprises an amino acid sequence encoded by the polynucleotide of SEQ ID NO:332; and the second polypeptide comprises an amino acid sequence encoded by the polynucleotide of SEQ ID NO:332.

In some aspects, VH1 and VH2 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; and VL1 and VL2 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 and VH2 comprise the amino acid sequence of SEQ ID NO:279; and VL1 and VL2 comprise the amino acid sequence of SEQ ID NO:275. In some aspects, L1 or L2 and L3 or L4 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L2 and L3 or L4 comprise the amino acid sequence of SEQ ID NO:27. In some aspects, L1 or L3 and L5 or L7 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L3 and L5 or L7 comprise the amino acid sequence of SEQ ID NO:27. In some aspects, L2 or L4 and L6 or L8 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L2 or L4 and L6 or L8 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some aspects, the sequence has a mutation at N62, for example, N62Q or N62Q at, for example, SEQ ID NO:279. In some preferred aspects, VH1, VH2, VL1 and VL2 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, VH1, VH2, VL1 and VL2 comprise or consist of the corresponding amino acid sequences specified above, optionally with (i) an Fc comprising or consisting of the corresponding amino acid sequence recited above; and/or (ii) one or more linker sequence as specified above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:333; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:333. In some aspects, both the first polypeptide and the second polypeptide have at least 90% identity to SEQ ID NO:333. In some aspects, the first polypeptide comprises the amino acid sequence of SEQ ID NO:333; and the second polypeptide comprises the amino acid sequence of SEQ ID NO:333. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:334; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:334. In some aspects, both the first polypeptide and the second polypeptide comprise an amino acid sequence encoded by a polynucleotide having at least 90% identity to SEQ ID NO:334. In some aspects, the first polypeptide comprises an amino acid sequence encoded by the polynucleotide of SEQ ID NO:334; and the second polypeptide comprises an amino acid sequence encoded by the polynucleotide of SEQ ID NO:334.

In some aspects, provided herein is an antigen binding polypeptide complex comprising a first polypeptide and a second polypeptide; wherein the first polypeptide has a structure represented by VL1-L1-VL2-L2-VH2-L3-VH1; VL1-L1-VH2-L2-VL2-L3-VH1; VH1-L4-VH2-L5-VL2-L6-VL1; or VH1-L4-VL2-L5-VH2-L6-VL1; wherein the second polypeptide has a structure represented by VL3-L7-VL4-L8-VH4-L9-VH3; VL3-L7-VH4-L8-VL4-L9-VH3; VH3-L10-VH4-L11-VL4-L12-VL3; or VH3-L10-VL4-L11-VH4-L12-VL3; wherein VL1 is a first immunoglobulin light chain variable region that specifically binds to a SARS-CoV-2 protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; and L1-L12 are amino acid linkers. In some aspects, provided herein is an antigen binding polypeptide complex comprising a first polypeptide and a second polypeptide; wherein the first polypeptide has a structure represented by VL1-L1-VL2-L2-VH2-L3-VH1-L4-Fc; VL1-L1-VH2-L2-VL2-L3-VH1-L4-Fc; VH1-L5-VH2-L6-VL2-L7-VL1-L8-Fc; or VH1-L5-VL2-L6-VH2-L7-VL1-L8-Fc; wherein the second polypeptide has a structure represented by Fc; VL3-L9-VL4-L10-VH4-L11-VH3-L12-Fc; VL3-L9-VH4-L10-VL4-L11-VH3-L12-Fc; VH3-L13-VH4-L14-VL4-L15-VL3-L16-Fc; or VH3-L13-VL4-L14-VH4-L15-VL3-L16-Fc; wherein VL1 is a first immunoglobulin light chain variable region that specifically binds to a SARS-CoV-2 protein; VL2 is a second immunoglobulin light chain variable region that specifically binds to a SARS-CoV-2 protein; VL3 is a third immunoglobulin light chain variable region that specifically binds to a SARS-CoV-2 protein; VL4 is a fourth immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; VH4 is a fourth immunoglobulin heavy chain variable region that specifically binds to a SARS-CoV-2 protein; Fc is a region comprising an immunoglobulin heavy chain constant region 2 (CH2), an immunoglobulin heavy chain constant region 3 (CH3), and optionally, an immunoglobulin hinge; and L1-L16 are amino acid linkers. In some aspects, provided herein is an antigen binding polypeptide complex comprising a first polypeptide and a second polypeptide; wherein the first polypeptide has a structure represented by VL1-L1-VL2-L2-VH2-L3-VH1; the second polypeptide has a structure represented by VL3-L7-VL4-L8-VH4-L9-VH3; VL3-L7-VH4-L8-VL4-L9-VH3; VH3-L10-VH4-L11-VL4-L12-VL3; or VH3-L10-VL4-L11-VH4-L12-VL3; and wherein the first and/or second polypeptide may have an Fe as shown above. In some aspects, provided herein is an antigen binding polypeptide complex comprising a first polypeptide and a second polypeptide; wherein the first polypeptide has a structure represented by VL1-L1-VH2-L2-VL2-L3-VH1; the second polypeptide has a structure represented by VL3-L7-VL4-L8-VH4-L9-VH3; VL3-L7-VH4-L8-VL4-L9-VH3; VH3-L10-VH4-L11-VL4-L12-VL3; or VH3-L10-VL4-L11-VH4-L12-VL3; and wherein the first and/or second polypeptide may have an Fc as shown above. In some aspects, provided herein is an antigen binding polypeptide complex comprising a first polypeptide and a second polypeptide; wherein the first polypeptide has a structure represented by VH1-L4-VH2-L5-VL2-L6-VL1; the second polypeptide has a structure represented by VL3-L7-VL4-L8-VH4-L9-VH3; VL3-L7-VH4-L8-VL4-L9-VH3; VH3-L10-VH4-L11-VL4-L12-VL3; or VH3-L10-VL4-L11-VH4-L12-VL3; and wherein the first and/or second polypeptide may have an Fc as shown above. In some aspects, provided herein is an antigen binding polypeptide complex comprising a first polypeptide and a second polypeptide; wherein the first polypeptide has a structure represented by VH1-L4-VL2-L5-VH2-L6-VL1; the second polypeptide has a structure represented by VL3-L7-VL4-L8-VH4-L9-VH3; VL3-L7-VH4-L8-VL4-L9-VH3; VH3-L10-VH4-L11-VL4-L12-VL3; or VH3-L10-VL4-L11-VH4-L12-VL3; and wherein the first and/or second polypeptide may have an Fc as shown above.

In some aspects, VH1, VH2, VH3 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:263; and VL1, VL2, VL3 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:259. In some aspects, VH1, VH2, VH3 and VH4 comprise the amino acid sequence of SEQ ID NO:263; and VL1, VL2, VL3 and VL4 comprise the amino acid sequence of SEQ ID NO:259. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:291; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:291. In some aspects, both the first polypeptide and the second polypeptide have at least 90% identity to SEQ ID NO:291. In some aspects, the first polypeptide comprises the amino acid sequence of SEQ ID NO:291; and the second polypeptide comprises the amino acid sequence of SEQ ID NO:291. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:292; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:292. In some aspects, both the first polypeptide and the second polypeptide comprise an amino acid sequence encoded by a polynucleotide having at least 90% identity to SEQ ID NO:292. In some aspects, the first polypeptide comprises an amino acid sequence encoded by the polynucleotide of SEQ ID NO:292; and the second polypeptide comprises an amino acid sequence encoded by the polynucleotide of SEQ ID NO:292.

In some aspects, VH1, VH2, VH3 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:271; and VL1, VL2, VL3 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:267. In some aspects, VH1, VH2, VH3 and VH4 comprise the amino acid sequence of SEQ ID NO:271; and VL1, VL2, VL3 and VL4 comprise the amino acid sequence of SEQ ID NO:267. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some preferred aspects, VH1, VH2, VH3, VH4, VL1, VL2, VL3 and VL4 have at least 90% identity with the corresponding amino acid sequences specified above, optionally with (i) an Fe having at least 90% identity with the corresponding amino acid sequence recited above; and/or (ii) one or more linker sequence as specified above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:295; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:295. In some aspects, both the first polypeptide and the second polypeptide have at least 90% identity to SEQ ID NO:295. In some aspects, the first polypeptide comprises the amino acid sequence of SEQ ID NO:295; and the second polypeptide comprises the amino acid sequence of SEQ ID NO:295. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:296; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:296. In some aspects, both the first polypeptide and the second polypeptide comprise an amino acid sequence encoded by a polynucleotide having at least 90% identity to SEQ ID NO:296. In some aspects, the first polypeptide comprises an amino acid sequence encoded by the polynucleotide of SEQ ID NO:296; and the second polypeptide comprises an amino acid sequence encoded by the polynucleotide of SEQ ID NO:296.

In some aspects, VH1, VH2, VH3 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; and VL1, VL2, VL3 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1, VH2, VH3 and VH4 comprise the amino acid sequence of SEQ ID NO:279; and VL1, VL2, VL3 and VL4 comprise the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:297; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:297. In some aspects, both the first polypeptide and the second polypeptide have at least 90% identity to SEQ ID NO:297. In some aspects, the first polypeptide comprises the amino acid sequence of SEQ ID NO:297; and the second polypeptide comprises the amino acid sequence of SEQ ID NO:297. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:298; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:298. In some aspects, both the first polypeptide and the second polypeptide comprise an amino acid sequence encoded by a polynucleotide having at least 90% identity to SEQ ID NO:298. In some aspects, the first polypeptide comprises an amino acid sequence encoded by the polynucleotide of SEQ ID NO:298; and the second polypeptide comprises an amino acid sequence encoded by the polynucleotide of SEQ ID NO:298.

In some aspects, VH1 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH2 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:263; VL1 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; and VL2 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:259. In some aspects, VH1 and VH3 comprise the amino acid sequence of SEQ ID NO:58; VH2 and VH4 comprise the amino acid sequence of SEQ ID NO:263; VL1 and VL3 comprise the amino acid sequence of SEQ ID NO:54; and VL2 and VL4 comprise the amino acid sequence of SEQ ID NO:259. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:299; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:299. In some aspects, both the first polypeptide and the second polypeptide have at least 90% identity to SEQ ID NO:299. In some aspects, the first polypeptide comprises the amino acid sequence of SEQ ID NO:299; and the second polypeptide comprises the amino acid sequence of SEQ ID NO:299. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:300; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:300. In some aspects, both the first polypeptide and the second polypeptide comprise an amino acid sequence encoded by a polynucleotide having at least 90% identity to SEQ ID NO:300. In some aspects, the first polypeptide comprises an amino acid sequence encoded by the polynucleotide of SEQ ID NO:300; and the second polypeptide comprises an amino acid sequence encoded by the polynucleotide of SEQ ID NO:300.

In some aspects, VH1 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VH2 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:263; VL1 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275; and VL2 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:259. In some aspects, VH1 and VH3 comprise the amino acid sequence of SEQ ID NO:279; VH2 and VH4 comprise the amino acid sequence of SEQ ID NO:263; VL1 and VL3 comprise the amino acid sequence of SEQ ID NO:275; and VL2 and VL4 comprise the amino acid sequence of SEQ ID NO:259. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:301; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:301. In some aspects, both the first polypeptide and the second polypeptide have at least 90% identity to SEQ ID NO:301. In some aspects, the first polypeptide comprises the amino acid sequence of SEQ ID NO:301; and the second polypeptide comprises the amino acid sequence of SEQ ID NO:301. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:302; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:302. In some aspects, both the first polypeptide and the second polypeptide comprise an amino acid sequence encoded by a polynucleotide having at least 90% identity to SEQ ID NO:302. In some aspects, the first polypeptide comprises an amino acid sequence encoded by the polynucleotide of SEQ ID NO:302; and the second polypeptide comprises an amino acid sequence encoded by the polynucleotide of SEQ ID NO:302.

In some aspects, VH1 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:263; VH2 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VL1 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:259; and VL2 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54. In some aspects, VH1 and VH3 comprise the amino acid sequence of SEQ ID NO:263; VH2 and VH4 comprise the amino acid sequence of SEQ ID NO:58; VL1 and VL3 comprise the amino acid sequence of SEQ ID NO:259; and VL2 and VL4 comprise the amino acid sequence of SEQ ID NO:54. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:303; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:303. In some aspects, both the first polypeptide and the second polypeptide have at least 90% identity to SEQ ID NO:303. In some aspects, the first polypeptide comprises the amino acid sequence of SEQ ID NO:303; and the second polypeptide comprises the amino acid sequence of SEQ ID NO:303. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:304; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:304. In some aspects, both the first polypeptide and the second polypeptide comprise an amino acid sequence encoded by a polynucleotide having at least 90% identity to SEQ ID NO:304. In some aspects, the first polypeptide comprises an amino acid sequence encoded by the polynucleotide of SEQ ID NO:304; and the second polypeptide comprises an amino acid sequence encoded by the polynucleotide of SEQ ID NO:304.

In some aspects, VH1 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:263; VH2 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:259; and VL2 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 and VH3 comprise the amino acid sequence of SEQ ID NO:263; VH2 and VH4 comprise the amino acid sequence of SEQ ID NO:279; VL1 and VL3 comprise the amino acid sequence of SEQ ID NO:259; and VL2 and VL4 comprise the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO: 27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:305; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:305. In some aspects, both the first polypeptide and the second polypeptide have at least 90% identity to SEQ ID NO:305. In some aspects, the first polypeptide comprises the amino acid sequence of SEQ ID NO:305; and the second polypeptide comprises the amino acid sequence of SEQ ID NO:305. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:306; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:306. In some aspects, both the first polypeptide and the second polypeptide comprise an amino acid sequence encoded by a polynucleotide having at least 90% identity to SEQ ID NO:306. In some aspects, the first polypeptide comprises an amino acid sequence encoded by the polynucleotide of SEQ ID NO:306; and the second polypeptide comprises an amino acid sequence encoded by the polynucleotide of SEQ ID NO:306.

In some aspects, VH1 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:263; VH2 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VL1 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:259; and VL2 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54. In some aspects, VH1 and VH4 comprise the amino acid sequence of SEQ ID NO:263; VH2 and VH3 comprise the amino acid sequence of SEQ ID NO:58; VL1 and VL4 comprise the amino acid sequence of SEQ ID NO:259; and VL2 and VL3 comprise the amino acid sequence of SEQ ID NO:54. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO: 27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:307; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:309. In some aspects, the first polypeptide has at least 90% identity to SEQ ID NO:307 and the second polypeptide has at least 90% identity to SEQ ID NO:309. In some aspects, the first polypeptide comprises the amino acid sequence of SEQ ID NO:307; and the second polypeptide comprises the amino acid sequence of SEQ ID NO:309. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:308; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:310. In some aspects, the first polypeptide comprises an amino acid sequence encoded by a polynucleotide having at least 90% identity to SEQ ID NO:308 and the second polypeptide comprises an amino acid sequence encoded by a polynucleotide having at least 90% identity to SEQ ID NO:310. In some aspects, the first polypeptide comprises an amino acid sequence encoded by the polynucleotide of SEQ ID NO:308; and the second polypeptide comprises an amino acid sequence encoded by the polynucleotide of SEQ ID NO:310.

In some aspects, VH1 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:263; VH2 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:259; and VL2 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 and VH4 comprise the amino acid sequence of SEQ ID NO:263; VH2 and VH3 comprise the amino acid sequence of SEQ ID NO:279; VL1 and VL4 comprise the amino acid sequence of SEQ ID NO:259; and VL2 and VL3 comprise the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:311; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:313. In some aspects, the first polypeptide has at least 90% identity to SEQ ID NO:311 and the second polypeptide has at least 90% identity to SEQ ID NO:313. In some aspects, the first polypeptide comprises the amino acid sequence of SEQ ID NO:311; and the second polypeptide comprises the amino acid sequence of SEQ ID NO:313. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:312; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:314. In some aspects, the first polypeptide comprises an amino acid sequence encoded by a polynucleotide having at least 90% identity to SEQ ID NO:312 and the second polypeptide comprises an amino acid sequence encoded by a polynucleotide having at least 90% identity to SEQ ID NO:314. In some aspects, the first polypeptide comprises an amino acid sequence encoded by the polynucleotide of SEQ ID NO:312; and the second polypeptide comprises an amino acid sequence encoded by the polynucleotide of SEQ ID NO:314.

In some aspects, VH1 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH2 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:271; VL1 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; and VL2 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:267. In some aspects, VH1 and VH3 comprise the amino acid sequence of SEQ ID NO:58; VH2 and VH4 comprise the amino acid sequence of SEQ ID NO:271; VL1 and VL3 comprise the amino acid sequence of SEQ ID NO:54; and VL2 and VL4 comprise the amino acid sequence of SEQ ID NO:267. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:315; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:315. In some aspects, both the first polypeptide and the second polypeptide have at least 90% identity to SEQ ID NO:315. In some aspects, the first polypeptide comprises the amino acid sequence of SEQ ID NO:315; and the second polypeptide comprises the amino acid sequence of SEQ ID NO:315. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:316; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:316. In some aspects, the first polypeptide comprises an amino acid sequence encoded by a polynucleotide having at least 90% identity to SEQ ID NO:316 and the second polypeptide comprises an amino acid sequence encoded by a polynucleotide having at least 90% identity to SEQ ID NO:316. In some aspects, the first polypeptide comprises an amino acid sequence encoded by the polynucleotide of SEQ ID NO:316; and the second polypeptide comprises an amino acid sequence encoded by the polynucleotide of SEQ ID NO:316.

In some aspects, VH1 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VH2 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:271; VL1 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275; and VL2 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:267. In some aspects, VH1 and VH3 comprise the amino acid sequence of SEQ ID NO:279; VH2 and VH4 comprise the amino acid sequence of SEQ ID NO:271; VL1 and VL3 comprise the amino acid sequence of SEQ ID NO:275; and VL2 and VL4 comprise the amino acid sequence of SEQ ID NO:267. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO: 27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:256; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:256. In some aspects, both the first polypeptide and the second polypeptide have at least 90% identity to SEQ ID NO:256. In some aspects, the first polypeptide comprises the amino acid sequence of SEQ ID NO:256; and the second polypeptide comprises the amino acid sequence of SEQ ID NO:256. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:507; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:507. In some aspects, both the first polypeptide and the second polypeptide comprise an amino acid sequence encoded by a polynucleotide having at least 90% identity to SEQ ID NO:507. In some aspects, the first polypeptide comprises an amino acid sequence encoded by the polynucleotide of SEQ ID NO:507; and the second polypeptide comprises an amino acid sequence encoded by the polynucleotide of SEQ ID NO:507.

In some aspects, VH1 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:271; VH2 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VL1 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:267; and VL2 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54. In some aspects, VH1 and VH3 comprise the amino acid sequence of SEQ ID NO:271; VH2 and VH4 comprise the amino acid sequence of SEQ ID NO:58; VL1 and VL3 comprise the amino acid sequence of SEQ ID NO:267; and VL2 and VL4 comprise the amino acid sequence of SEQ ID NO:54. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:317; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:317. In some aspects, both the first polypeptide and the second polypeptide have at least 90% identity to SEQ ID NO:317. In some aspects, the first polypeptide comprises the amino acid sequence of SEQ ID NO:317; and the second polypeptide comprises the amino acid sequence of SEQ ID NO:317. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:318; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:318. In some aspects, both the first polypeptide and the second polypeptide comprise an amino acid sequence encoded by a polynucleotide having at least 90% identity to SEQ ID NO:318. In some aspects, the first polypeptide comprises an amino acid sequence encoded by the polynucleotide of SEQ ID NO:318; and the second polypeptide comprises an amino acid sequence encoded by the polynucleotide of SEQ ID NO:318.

In some aspects, VH1 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:271; VH2 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:267; and VL2 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 and VH3 comprise the amino acid sequence of SEQ ID NO:271; VH2 and VH4 comprise the amino acid sequence of SEQ ID NO:279; VL1 and VL3 comprise the amino acid sequence of SEQ ID NO:267; and VL2 and VL4 comprise the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO: 27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:319; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:319. In some aspects, both the first polypeptide and the second polypeptide have at least 90% identity to SEQ ID NO:319. In some aspects, the first polypeptide comprises the amino acid sequence of SEQ ID NO:319; and the second polypeptide comprises the amino acid sequence of SEQ ID NO:319. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:320; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:320. In some aspects, both the first polypeptide and the second polypeptide comprise an amino acid sequence encoded by a polynucleotide having at least 90% identity to SEQ ID NO:320. In some aspects, the first polypeptide comprises an amino acid sequence encoded by the polynucleotide of SEQ ID NO:320; and the second polypeptide comprises an amino acid sequence encoded by the polynucleotide of SEQ ID NO:320.

In some aspects, VH1 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:271; VH2 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VL1 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:267; and VL2 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54. In some aspects, VH1 and VH4 comprise the amino acid sequence of SEQ ID NO:271; VH2 and VH3 comprise the amino acid sequence of SEQ ID NO:58; VL1 and VL4 comprise the amino acid sequence of SEQ ID NO:267; and VL2 and VL3 comprise the amino acid sequence of SEQ ID NO:54. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; and/or (ii) one or more linker sequence as specified above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; and/or (ii) one or more linker sequence as specified above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:321; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:323. In some aspects, the first polypeptide has at least 90% identity to SEQ ID NO:321 and the second polypeptide has at least 90% identity to SEQ ID NO:323. In some aspects, the first polypeptide comprises the amino acid sequence of SEQ ID NO:321; and the second polypeptide comprises the amino acid sequence of SEQ ID NO:323. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:322; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:324. In some aspects, the first polypeptide comprises an amino acid sequence encoded by a polynucleotide having at least 90% identity to SEQ ID NO:322 and the second polypeptide comprises an amino acid sequence encoded by a polynucleotide having at least 90% identity to SEQ ID NO:324. In some aspects, the first polypeptide comprises an amino acid sequence encoded by the polynucleotide of SEQ ID NO:322; and the second polypeptide comprises an amino acid sequence encoded by the polynucleotide of SEQ ID NO:324.

In some aspects, VH1 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:271; VH2 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:267; and VL2 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 and VH4 comprise the amino acid sequence of SEQ ID NO:271; VH2 and VH3 comprise the amino acid sequence of SEQ ID NO:279; VL1 and VL4 comprise the amino acid sequence of SEQ ID NO:267; and VL2 and VL3 comprise the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:325; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:327. In some aspects, the first polypeptide has at least 90% identity to SEQ ID NO:325 and the second polypeptide has at least 90% identity to SEQ ID NO:327. In some aspects, the first polypeptide comprises the amino acid sequence of SEQ ID NO:325; and the second polypeptide comprises the amino acid sequence of SEQ ID NO:327. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:326; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:328. In some aspects, the first polypeptide comprises an amino acid sequence encoded by a polynucleotide having at least 90% identity to SEQ ID NO:326 and the second polypeptide comprises an amino acid sequence encoded by a polynucleotide having at least 90% identity to SEQ ID NO:328. In some aspects, the first polypeptide comprises an amino acid sequence encoded by the polynucleotide of SEQ ID NO:326; and the second polypeptide comprises an amino acid sequence encoded by the polynucleotide of SEQ ID NO:328.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VH2 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:287; VH4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VL1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275; VL2 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:283; and VL4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54. In some aspects, VH1 comprises the amino acid sequence of SEQ ID NO:279; VH2 and VH3 comprise the amino acid sequence of SEQ ID NO:287; VH4 comprises the amino acid sequence of SEQ ID NO:58; VL1 comprises the amino acid sequence of SEQ ID NO:275; VL2 and VL3 comprise the amino acid sequence of SEQ ID NO:283; and VL4 comprises the amino acid sequence of SEQ ID NO:54. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:335; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:337. In some aspects, the first polypeptide has at least 90% identity to SEQ ID NO:335 and the second polypeptide has at least 90% identity to SEQ ID NO:337. In some aspects, the first polypeptide comprises the amino acid sequence of SEQ ID NO:335; and the second polypeptide comprises the amino acid sequence of SEQ ID NO:337. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:336; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:338. In some aspects, the first polypeptide comprises an amino acid sequence encoded by a polynucleotide having at least 90% identity to SEQ ID NO:336 and the second polypeptide comprises an amino acid sequence encoded by a polynucleotide having at least 90% identity to SEQ ID NO:338. In some aspects, the first polypeptide comprises an amino acid sequence encoded by the polynucleotide of SEQ ID NO:336; and the second polypeptide comprises an amino acid sequence encoded by the polynucleotide of SEQ ID NO:338.

In some aspects, VH1 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:287; VH2 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:283; and VL2 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 and VH3 comprise the amino acid sequence of SEQ ID NO:287; VH2 and VH4 comprise the amino acid sequence of SEQ ID NO:279; VL1 and VL3 comprise the amino acid sequence of SEQ ID NO:283; and VL2 and VL4 comprise the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO: 6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:37. In some aspects, Fc comprises the amino acid sequence of SEQ ID NO:37. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:339; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:339. In some aspects, both the first polypeptide and the second polypeptide have at least 90% identity to SEQ ID NO:339. In some aspects, the first polypeptide comprises the amino acid sequence of SEQ ID NO:339; and the second polypeptide comprises the amino acid sequence of SEQ ID NO:339. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:340; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:340. In some aspects, both the first polypeptide and the second polypeptide comprise an amino acid sequence encoded by a polynucleotide having at least 90% identity to SEQ ID NO:340. In some aspects, the first polypeptide comprises an amino acid sequence encoded by the polynucleotide of SEQ ID NO:340; and the second polypeptide comprises an amino acid sequence encoded by the polynucleotide of SEQ ID NO:340.

In some aspects, VH1 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:287; VH2 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:283; and VL2 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 and VH4 comprise the amino acid sequence of SEQ ID NO:287; VH2 and VH3 comprise the amino acid sequence of SEQ ID NO:279; VL1 and VL4 comprise the amino acid sequence of SEQ ID NO:283; and VL2 and VL3 comprise the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293-294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:341; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:343. In some aspects, the first polypeptide has at least 90% identity to SEQ ID NO:341 and the second polypeptide has at least 90% identity to SEQ ID NO:343. In some aspects, the first polypeptide comprises the amino acid sequence of SEQ ID NO:341; and the second polypeptide comprises the amino acid sequence of SEQ ID NO:343. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:342; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:344. In some aspects, the first polypeptide comprises an amino acid sequence encoded by a polynucleotide having at least 90% identity to SEQ ID NO:342 and the second polypeptide comprises an amino acid sequence encoded by a polynucleotide having at least 90% identity to SEQ ID NO:344. In some aspects, the first polypeptide comprises an amino acid sequence encoded by the polynucleotide of SEQ ID NO:342; and the second polypeptide comprises an amino acid sequence encoded by the polynucleotide of SEQ ID NO:344.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH2 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:263; VH4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; VL2 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:259; and VL4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 comprises the amino acid sequence of SEQ ID NO:58; VH2 and VH3 comprise the amino acid sequence of SEQ ID NO:263; VH4 comprises the amino acid sequence of SEQ ID NO:279; VL1 comprises the amino acid sequence of SEQ ID NO:54; VL2 and VL3 comprise the amino acid sequence of SEQ ID NO:259; and VL4 comprises the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:345; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:347. In some aspects, the first polypeptide has at least 90% identity to SEQ ID NO:345 and the second polypeptide has at least 90% identity to SEQ ID NO:347. In some aspects, the first polypeptide comprises the amino acid sequence of SEQ ID NO:345; and the second polypeptide comprises the amino acid sequence of SEQ ID NO:347. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:346; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:348. In some aspects, the first polypeptide comprises an amino acid sequence encoded by a polynucleotide having at least 90% identity to SEQ ID NO:346 and the second polypeptide comprises an amino acid sequence encoded by a polynucleotide having at least 90% identity to SEQ ID NO:348. In some aspects, the first polypeptide comprises an amino acid sequence encoded by the polynucleotide of SEQ ID NO:346; and the second polypeptide comprises an amino acid sequence encoded by the polynucleotide of SEQ ID NO:348.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH2 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:263; VH3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; VL2 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:259; and VL3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 comprises the amino acid sequence of SEQ ID NO:58; VH2 and VH4 comprise the amino acid sequence of SEQ ID NO:263; VH3 comprises the amino acid sequence of SEQ ID NO:279; VL1 comprises the amino acid sequence of SEQ ID NO:54; VL2 and VL4 comprise the amino acid sequence of SEQ ID NO:259; and VL3 comprises the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:349; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:351. In some aspects, the first polypeptide has at least 90% identity to SEQ ID NO:349 and the second polypeptide has at least 90% identity to SEQ ID NO:351. In some aspects, the first polypeptide comprises the amino acid sequence of SEQ ID NO:349; and the second polypeptide comprises the amino acid sequence of SEQ ID NO:351. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:350; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:352. In some aspects, the first polypeptide comprises an amino acid sequence encoded by a polynucleotide having at least 90% identity to SEQ ID NO:350 and the second polypeptide comprises an amino acid sequence encoded by a polynucleotide having at least 90% identity to SEQ ID NO:352. In some aspects, the first polypeptide comprises an amino acid sequence encoded by the polynucleotide of SEQ ID NO:350; and the second polypeptide comprises an amino acid sequence encoded by the polynucleotide of SEQ ID NO:352.

In some aspects, VH1 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:263; VH2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:259; VL2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; and VL4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 and VH3 comprise the amino acid sequence of SEQ ID NO:263; VH2 comprises the amino acid sequence of SEQ ID NO:58; VH4 comprises the amino acid sequence of SEQ ID NO:279; VL1 and VL3 comprise the amino acid sequence of SEQ ID NO:259; VL2 comprises the amino acid sequence of SEQ ID NO:54; and VL4 comprises the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:353; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:355. In some aspects, the first polypeptide has at least 90% identity to SEQ ID NO:353 and the second polypeptide has at least 90% identity to SEQ ID NO:355. In some aspects, the first polypeptide comprises the amino acid sequence of SEQ ID NO:353; and the second polypeptide comprises the amino acid sequence of SEQ ID NO:355. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:354; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:356. In some aspects, the first polypeptide comprises an amino acid sequence encoded by a polynucleotide having at least 90% identity to SEQ ID NO:354 and the second polypeptide comprises an amino acid sequence encoded by a polynucleotide having at least 90% identity to SEQ ID NO:356. In some aspects, the first polypeptide comprises an amino acid sequence encoded by the polynucleotide of SEQ ID NO:354; and the second polypeptide comprises an amino acid sequence encoded by the polynucleotide of SEQ ID NO:356.

In some aspects, VH1 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:263; VH2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:259; VL2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; and VL3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 and VH4 comprise the amino acid sequence of SEQ ID NO:263; VH2 comprises the amino acid sequence of SEQ ID NO:58; VH3 comprises the amino acid sequence of SEQ ID NO:279; VL1 and VL4 comprise the amino acid sequence of SEQ ID NO:259; VL2 comprises the amino acid sequence of SEQ ID NO:54; and VL3 comprises the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:357; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:359. In some aspects, the first polypeptide has at least 90% identity to SEQ ID NO:357 and the second polypeptide has at least 90% identity to SEQ ID NO:359. In some aspects, the first polypeptide comprises the amino acid sequence of SEQ ID NO:357; and the second polypeptide comprises the amino acid sequence of SEQ ID NO:359. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:358; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:360. In some aspects, the first polypeptide comprises an amino acid sequence encoded by a polynucleotide having at least 90% identity to SEQ ID NO:358 and the second polypeptide comprises an amino acid sequence encoded by a polynucleotide having at least 90% identity to SEQ ID NO:360. In some aspects, the first polypeptide comprises an amino acid sequence encoded by the polynucleotide of SEQ ID NO:358; and the second polypeptide comprises an amino acid sequence encoded by the polynucleotide of SEQ ID NO:360.

In some aspects, VH1 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:263; VH4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; VL2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:259; and VL4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 and VH3 comprise the amino acid sequence of SEQ ID NO:58; VH2 comprises the amino acid sequence of SEQ ID NO:263; VH4 comprises the amino acid sequence of SEQ ID NO:279; VL1 and VL3 comprise the amino acid sequence of SEQ ID NO:54; VL2 comprises the amino acid sequence of SEQ ID NO:259; and VL4 comprises the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:361; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:363. In some aspects, the first polypeptide has at least 90% identity to SEQ ID NO:361 and the second polypeptide has at least 90% identity to SEQ ID NO:363. In some aspects, the first polypeptide comprises the amino acid sequence of SEQ ID NO:361; and the second polypeptide comprises the amino acid sequence of SEQ ID NO:363. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:362; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:364. In some aspects, the first polypeptide comprises an amino acid sequence encoded by a polynucleotide having at least 90% identity to SEQ ID NO:362 and the second polypeptide comprises an amino acid sequence encoded by a polynucleotide having at least 90% identity to SEQ ID NO:364. In some aspects, the first polypeptide comprises an amino acid sequence encoded by the polynucleotide of SEQ ID NO:362; and the second polypeptide comprises an amino acid sequence encoded by the polynucleotide of SEQ ID NO:364.

In some aspects, VH1 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:263; VH3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; VL2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:259; and VL3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 and VH4 comprise the amino acid sequence of SEQ ID NO:58; VH2 comprises the amino acid sequence of SEQ ID NO:263; VH3 comprises the amino acid sequence of SEQ ID NO:279; VL1 and VL4 comprise the amino acid sequence of SEQ ID NO:54; VL2 comprises the amino acid sequence of SEQ ID NO:259; and VL3 comprises the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:365; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:367. In some aspects, the first polypeptide has at least 90% identity to SEQ ID NO:365 and the second polypeptide has at least 90% identity to SEQ ID NO:367. In some aspects, the first polypeptide comprises the amino acid sequence of SEQ ID NO:365; and the second polypeptide comprises the amino acid sequence of SEQ ID NO:367. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:366; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:368. In some aspects, the first polypeptide comprises an amino acid sequence encoded by a polynucleotide having at least 90% identity to SEQ ID NO:366 and the second polypeptide comprises an amino acid sequence encoded by a polynucleotide having at least 90% identity to SEQ ID NO:368. In some aspects, the first polypeptide comprises an amino acid sequence encoded by the polynucleotide of SEQ ID NO:366; and the second polypeptide comprises an amino acid sequence encoded by the polynucleotide of SEQ ID NO:368.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:263; VH2 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:259; VL2 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; and VL4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 comprises the amino acid sequence of SEQ ID NO:263; VH2 and VH3 comprise the amino acid sequence of SEQ ID NO:58; VH4 comprises the amino acid sequence of SEQ ID NO:279; VL1 comprises the amino acid sequence of SEQ ID NO:259; VL2 and VL3 comprise the amino acid sequence of SEQ ID NO:54; and VL4 comprises the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:369; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:371. In some aspects, the first polypeptide comprises the amino acid sequence of SEQ ID NO:369; and the second polypeptide comprises the amino acid sequence of SEQ ID NO:371. In some aspects, the first polypeptide has at least 90% identity to SEQ ID NO:369 and the second polypeptide has at least 90% identity to SEQ ID NO:371. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:370; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:372. In some aspects, the first polypeptide comprises an amino acid sequence encoded by a polynucleotide having at least 90% identity to SEQ ID NO:370 and the second polypeptide comprises an amino acid sequence encoded by a polynucleotide having at least 90% identity to SEQ ID NO:372. In some aspects, the first polypeptide comprises an amino acid sequence encoded by the polynucleotide of SEQ ID NO:370; and the second polypeptide comprises an amino acid sequence encoded by the polynucleotide of SEQ ID NO:372.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:263; VH2 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:259; VL2 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; and VL3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 comprises the amino acid sequence of SEQ ID NO:263; VH2 and VH4 comprise the amino acid sequence of SEQ ID NO:58; VH3 comprises the amino acid sequence of SEQ ID NO:279; VL1 comprises the amino acid sequence of SEQ ID NO:259; VL2 and VL4 comprise the amino acid sequence of SEQ ID NO:54; and VL3 comprises the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:373; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:375. In some aspects, the first polypeptide has at least 90% identity to SEQ ID NO:373 and the second polypeptide has at least 90% identity to SEQ ID NO:375. In some aspects, the first polypeptide comprises the amino acid sequence of SEQ ID NO:373; and the second polypeptide comprises the amino acid sequence of SEQ ID NO:375. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:374; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:376. In some aspects, the first polypeptide comprises an amino acid sequence encoded by a polynucleotide having at least 90% identity to SEQ ID NO:374 and the second polypeptide comprises an amino acid sequence encoded by a polynucleotide having at least 90% identity to SEQ ID NO:376. In some aspects, the first polypeptide comprises an amino acid sequence encoded by the polynucleotide of SEQ ID NO:374; and the second polypeptide comprises an amino acid sequence encoded by the polynucleotide of SEQ ID NO:376.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:263; VH2 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VH4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VL1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:259; VL2 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275; and VL4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54. In some aspects, VH1 comprises the amino acid sequence of SEQ ID NO:263; VH2 and VH3 comprise the amino acid sequence of SEQ ID NO:279; VH4 comprises the amino acid sequence of SEQ ID NO:58; VL1 comprises the amino acid sequence of SEQ ID NO:259; VL2 and VL3 comprise the amino acid sequence of SEQ ID NO:275; and VL4 comprises the amino acid sequence of SEQ ID NO:54. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:377; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:379. In some aspects, the first polypeptide has at least 90% identity to SEQ ID NO:377 and the second polypeptide has at least 90% identity to SEQ ID NO:379. In some aspects, the first polypeptide comprises the amino acid sequence of SEQ ID NO:377; and the second polypeptide comprises the amino acid sequence of SEQ ID NO:379. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:378; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:380. In some aspects, the first polypeptide comprises an amino acid sequence encoded by a polynucleotide having at least 90% identity to SEQ ID NO:378 and the second polypeptide comprises an amino acid sequence encoded by a polynucleotide having at least 90% identity to SEQ ID NO:380. In some aspects, the first polypeptide comprises an amino acid sequence encoded by the polynucleotide of SEQ ID NO:378; and the second polypeptide comprises an amino acid sequence encoded by the polynucleotide of SEQ ID NO:380.

In some aspects, VH1 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VH2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:263; VH4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VL1 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275; VL2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:259; and VL4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54. In some aspects, VH1 and VH3 comprise the amino acid sequence of SEQ ID NO:279; VH2 comprises the amino acid sequence of SEQ ID NO:263; VH4 comprises the amino acid sequence of SEQ ID NO:58; VL1 and VL3 comprise the amino acid sequence of SEQ ID NO:275; VL2 comprises the amino acid sequence of SEQ ID NO:259; and VL4 comprises the amino acid sequence of SEQ ID NO:54. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fe effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:381; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:383. In some aspects, the first polypeptide has at least 90% identity to SEQ ID NO:381 and the second polypeptide has at least 90% identity to SEQ ID NO:383. In some aspects, the first polypeptide comprises the amino acid sequence of SEQ ID NO:381; and the second polypeptide comprises the amino acid sequence of SEQ ID NO:383. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:382; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:384. In some aspects, the first polypeptide comprises an amino acid sequence encoded by a polynucleotide having at least 90% identity to SEQ ID NO:382 and the second polypeptide comprises an amino acid sequence encoded by a polynucleotide having at least 90% identity to SEQ ID NO:384. In some aspects, the first polypeptide comprises an amino acid sequence encoded by the polynucleotide of SEQ ID NO:382; and the second polypeptide comprises an amino acid sequence encoded by the polynucleotide of SEQ ID NO:384.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH2 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VH3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:263; VL1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; VL2 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275; and VL3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:259. In some aspects, VH1 comprises the amino acid sequence of SEQ ID NO:58; VH2 and VH4 comprise the amino acid sequence of SEQ ID NO:279; VH3 comprises the amino acid sequence of SEQ ID NO:263; VL1 comprises the amino acid sequence of SEQ ID NO:54; VL2 and VL4 comprise the amino acid sequence of SEQ ID NO:275; and VL3 comprises the amino acid sequence of SEQ ID NO:259. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:385; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:387. In some aspects, the first polypeptide has at least 90% identity to SEQ ID NO:385 and the second polypeptide has at least 90% identity to SEQ ID NO:387. In some aspects, the first polypeptide comprises the amino acid sequence of SEQ ID NO:385; and the second polypeptide comprises the amino acid sequence of SEQ ID NO:387. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:386; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:388. In some aspects, the first polypeptide comprises an amino acid sequence encoded by a polynucleotide having at least 90% identity to SEQ ID NO:386 and the second polypeptide comprises an amino acid sequence encoded by a polynucleotide having at least 90% identity to SEQ ID NO:388. In some aspects, the first polypeptide comprises an amino acid sequence encoded by the polynucleotide of SEQ ID NO:386; and the second polypeptide comprises an amino acid sequence encoded by the polynucleotide of SEQ ID NO:388.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH2 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VH4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:263; VL1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; VL2 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275; and VL4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:259. In some aspects, VH1 comprises the amino acid sequence of SEQ ID NO:58; VH2 and VH3 comprise the amino acid sequence of SEQ ID NO:279; VH4 comprises the amino acid sequence of SEQ ID NO:263; VL1 comprises the amino acid sequence of SEQ ID NO:54; VL2 and VL3 comprise the amino acid sequence of SEQ ID NO:275; and VL4 comprises the amino acid sequence of SEQ ID NO:259. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:389; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:391. In some aspects, the first polypeptide has at least 90% identity to SEQ ID NO:389 and the second polypeptide has at least 90% identity to SEQ ID NO:391. In some aspects, the first polypeptide comprises the amino acid sequence of SEQ ID NO:389; and the second polypeptide comprises the amino acid sequence of SEQ ID NO:391. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:390; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:392. In some aspects, the first polypeptide comprises an amino acid sequence encoded by a polynucleotide having at least 90% identity to SEQ ID NO:390 and the second polypeptide comprises an amino acid sequence encoded by a polynucleotide having at least 90% identity to SEQ ID NO:392. In some aspects, the first polypeptide comprises an amino acid sequence encoded by the polynucleotide of SEQ ID NO:390; and the second polypeptide comprises an amino acid sequence encoded by the polynucleotide of SEQ ID NO:392.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH2 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:271; VH4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; VL2 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:267; and VL4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 comprises the amino acid sequence of SEQ ID NO:58; VH2 and VH3 comprise the amino acid sequence of SEQ ID NO:271; VH4 comprises the amino acid sequence of SEQ ID NO:279; VL1 comprises the amino acid sequence of SEQ ID NO:54; VL2 and VL3 comprise the amino acid sequence of SEQ ID NO:267; and VL4 comprises the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:257; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:258. In some aspects, the first polypeptide has at least 90% identity to SEQ ID NO:257 and the second polypeptide has at least 90% identity to SEQ ID NO:258. In some aspects, the first polypeptide comprises the amino acid sequence of SEQ ID NO:257; and the second polypeptide comprises the amino acid sequence of SEQ ID NO:258. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:393; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:394. In some aspects, the first polypeptide comprises an amino acid sequence encoded by a polynucleotide having at least 90% identity to SEQ ID NO:393 and the second polypeptide comprises an amino acid sequence encoded by a polynucleotide having at least 90% identity to SEQ ID NO:394. In some aspects, the first polypeptide comprises an amino acid sequence encoded by the polynucleotide of SEQ ID NO:393; and the second polypeptide comprises an amino acid sequence encoded by the polynucleotide of SEQ ID NO:394.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH2 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:271; VH3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; VL2 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:267; and VL3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 comprises the amino acid sequence of SEQ ID NO:58; VH2 and VH4 comprise the amino acid sequence of SEQ ID NO:271; VH3 comprises the amino acid sequence of SEQ ID NO:279; VL1 comprises the amino acid sequence of SEQ ID NO:54; VL2 and VL4 comprise the amino acid sequence of SEQ ID NO:267; and VL3 comprises the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:395; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:397. In some aspects, the first polypeptide has at least 90% identity to SEQ ID NO:395 and the second polypeptide has at least 90% identity to SEQ ID NO:397. In some aspects, the first polypeptide comprises the amino acid sequence of SEQ ID NO:395; and the second polypeptide comprises the amino acid sequence of SEQ ID NO:397. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:396; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:398. In some aspects, the first polypeptide comprises an amino acid sequence encoded by a polynucleotide having at least 90% identity to SEQ ID NO:396 and the second polypeptide comprises an amino acid sequence encoded by a polynucleotide having at least 90% identity to SEQ ID NO:398. In some aspects, the first polypeptide comprises an amino acid sequence encoded by the polynucleotide of SEQ ID NO:396; and the second polypeptide comprises an amino acid sequence encoded by the polynucleotide of SEQ ID NO:398.

In some aspects, VH1 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:271; VH2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:267; VL2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; and VL4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 and VH3 comprise the amino acid sequence of SEQ ID NO:271; VH2 comprises the amino acid sequence of SEQ ID NO:58; VH4 comprises the amino acid sequence of SEQ ID NO:279; VL1 and VL3 comprise the amino acid sequence of SEQ ID NO:267; VL2 comprises the amino acid sequence of SEQ ID NO:54; and VL4 comprises the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:399; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:401. In some aspects, the first polypeptide has at least 90% identity to SEQ ID NO:399 and the second polypeptide has at least 90% identity to SEQ ID NO:401. In some aspects, the first polypeptide comprises the amino acid sequence of SEQ ID NO:399; and the second polypeptide comprises the amino acid sequence of SEQ ID NO:401. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:400; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:402. In some aspects, the first polypeptide comprises an amino acid sequence encoded by a polynucleotide having at least 90% identity to SEQ ID NO:400 and the second polypeptide comprises an amino acid sequence encoded by a polynucleotide having at least 90% identity to SEQ ID NO:402. In some aspects, the first polypeptide comprises an amino acid sequence encoded by the polynucleotide of SEQ ID NO:400; and the second polypeptide comprises an amino acid sequence encoded by the polynucleotide of SEQ ID NO:402.

In some aspects, VH1 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:271; VH2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:267; VL2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; and VL3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 and VH4 comprise the amino acid sequence of SEQ ID NO:271; VH2 comprises the amino acid sequence of SEQ ID NO:58; VH3 comprises the amino acid sequence of SEQ ID NO:279; VL1 and VL4 comprise the amino acid sequence of SEQ ID NO:267; VL2 comprises the amino acid sequence of SEQ ID NO:54; and VL3 comprises the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:403; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:405. In some aspects, the first polypeptide has at least 90% identity to SEQ ID NO:403 and the second polypeptide has at least 90% identity to SEQ ID NO:405. In some aspects, the first polypeptide comprises the amino acid sequence of SEQ ID NO:403; and the second polypeptide comprises the amino acid sequence of SEQ ID NO:405. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:404; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:406. In some aspects, the first polypeptide comprises an amino acid sequence encoded by a polynucleotide having at least 90% identity to SEQ ID NO:404 and the second polypeptide comprises an amino acid sequence encoded by a polynucleotide having at least 90% identity to SEQ ID NO:406. In some aspects, the first polypeptide comprises an amino acid sequence encoded by the polynucleotide of SEQ ID NO:404; and the second polypeptide comprises an amino acid sequence encoded by the polynucleotide of SEQ ID NO:406.

In some aspects, VH1 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:271; VH4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; VL2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:267; and VL4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 and VH3 comprise the amino acid sequence of SEQ ID NO:58; VH2 comprises the amino acid sequence of SEQ ID NO:271; VH4 comprises the amino acid sequence of SEQ ID NO:279; VL1 and VL3 comprise the amino acid sequence of SEQ ID NO:54; VL2 comprises the amino acid sequence of SEQ ID NO:267; and VL4 comprises the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:407; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:409. In some aspects, the first polypeptide has at least 90% identity to SEQ ID NO:407 and the second polypeptide has at least 90% identity to SEQ ID NO:409. In some aspects, the first polypeptide comprises the amino acid sequence of SEQ ID NO:407; and the second polypeptide comprises the amino acid sequence of SEQ ID NO:409. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:408; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:410. In some aspects, the first polypeptide comprises an amino acid sequence encoded by a polynucleotide having at least 90% identity to SEQ ID NO:408 and the second polypeptide comprises an amino acid sequence encoded by a polynucleotide having at least 90% identity to SEQ ID NO:410. In some aspects, the first polypeptide comprises an amino acid sequence encoded by the polynucleotide of SEQ ID NO:408; and the second polypeptide comprises an amino acid sequence encoded by the polynucleotide of SEQ ID NO:410.

In some aspects, VH1 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:271; VH3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; VL2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:267; and VL3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 and VH4 comprise the amino acid sequence of SEQ ID NO:58; VH2 comprises the amino acid sequence of SEQ ID NO:271; VH3 comprises the amino acid sequence of SEQ ID NO:279; VL1 and VL4 comprise the amino acid sequence of SEQ ID NO:54; VL2 comprises the amino acid sequence of SEQ ID NO:267; and VL3 comprises the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:411; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:413. In some aspects, the first polypeptide has at least 90% identity to SEQ ID NO:411 and the second polypeptide has at least 90% identity to SEQ ID NO:413. In some aspects, the first polypeptide comprises the amino acid sequence of SEQ ID NO:411; and the second polypeptide comprises the amino acid sequence of SEQ ID NO:413. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:412; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:414. In some aspects, the first polypeptide comprises an amino acid sequence encoded by a polynucleotide having at least 90% identity to SEQ ID NO:412 and the second polypeptide comprises an amino acid sequence encoded by a polynucleotide having at least 90% identity to SEQ ID NO:414. In some aspects, the first polypeptide comprises an amino acid sequence encoded by the polynucleotide of SEQ ID NO:412; and the second polypeptide comprises an amino acid sequence encoded by the polynucleotide of SEQ ID NO:414.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:271; VH2 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:267; VL2 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; and VL4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 comprises the amino acid sequence of SEQ ID NO:271; VH2 and VH3 comprise the amino acid sequence of SEQ ID NO:58; VH4 comprises the amino acid sequence of SEQ ID NO:279; VL1 comprises the amino acid sequence of SEQ ID NO:267; VL2 and VL3 comprise the amino acid sequence of SEQ ID NO:54; and VL4 comprises the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:415; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:417. In some aspects, the first polypeptide has at least 90% identity to SEQ ID NO:415 and the second polypeptide has at least 90% identity to SEQ ID NO:417. In some aspects, the first polypeptide comprises the amino acid sequence of SEQ ID NO:415; and the second polypeptide comprises the amino acid sequence of SEQ ID NO:417. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:416; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:418. In some aspects, the first polypeptide comprises an amino acid sequence encoded by a polynucleotide having at least 90% identity to SEQ ID NO:416 and the second polypeptide comprises an amino acid sequence encoded by a polynucleotide having at least 90% identity to SEQ ID NO:418. In some aspects, the first polypeptide comprises an amino acid sequence encoded by the polynucleotide of SEQ ID NO:416; and the second polypeptide comprises an amino acid sequence encoded by the polynucleotide of SEQ ID NO:418.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:271; VH2 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:267; VL2 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; and VL3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 comprises the amino acid sequence of SEQ ID NO:271; VH2 and VH4 comprise the amino acid sequence of SEQ ID NO:58; VH3 comprises the amino acid sequence of SEQ ID NO:279; VL1 comprises the amino acid sequence of SEQ ID NO:267; VL2 and VL4 comprise the amino acid sequence of SEQ ID NO:54; and VL3 comprises the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:419; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:421. In some aspects, the first polypeptide has at least 90% identity to SEQ ID NO:419 and the second polypeptide has at least 90% identity to SEQ ID NO:421. In some aspects, the first polypeptide comprises the amino acid sequence of SEQ ID NO:419; and the second polypeptide comprises the amino acid sequence of SEQ ID NO:421. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:420; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:422. In some aspects, the first polypeptide comprises an amino acid sequence encoded by a polynucleotide having at least 90% identity to SEQ ID NO:420 and the second polypeptide comprises an amino acid sequence encoded by a polynucleotide having at least 90% identity to SEQ ID NO:422. In some aspects, the first polypeptide comprises an amino acid sequence encoded by the polynucleotide of SEQ ID NO:420; and the second polypeptide comprises an amino acid sequence encoded by the polynucleotide of SEQ ID NO:422.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:271; VH2 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VH4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VL1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:267; VL2 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275; and VL4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54. In some aspects, VH1 comprises the amino acid sequence of SEQ ID NO:271; VH2 and VH3 comprise the amino acid sequence of SEQ ID NO:279; VH4 comprises the amino acid sequence of SEQ ID NO:58; VL1 comprises the amino acid sequence of SEQ ID NO:267; VL2 and VL3 comprise the amino acid sequence of SEQ ID NO:275; and VL4 comprises the amino acid sequence of SEQ ID NO:54. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fe effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:423; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:425. In some aspects, the first polypeptide has at least 90% identity to SEQ ID NO:423 and the second polypeptide has at least 90% identity to SEQ ID NO:425. In some aspects, the first polypeptide comprises the amino acid sequence of SEQ ID NO:423; and the second polypeptide comprises the amino acid sequence of SEQ ID NO:425. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:424; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:426. In some aspects, the first polypeptide comprises an amino acid sequence encoded by a polynucleotide having at least 90% identity to SEQ ID NO:424 and the second polypeptide comprises an amino acid sequence encoded by a polynucleotide having at least 90% identity to SEQ ID NO:426. In some aspects, the first polypeptide comprises an amino acid sequence encoded by the polynucleotide of SEQ ID NO:424; and the second polypeptide comprises an amino acid sequence encoded by the polynucleotide of SEQ ID NO:426.

In some aspects, VH2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:271; VH1 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VH4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VL2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:267; VL1 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275; and VL4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54. In some aspects, VH2 comprises the amino acid sequence of SEQ ID NO:271; VH1 and VH3 comprise the amino acid sequence of SEQ ID NO:279; VH4 comprises the amino acid sequence of SEQ ID NO:58; VL2 comprises the amino acid sequence of SEQ ID NO:267; VL1 and VL3 comprise the amino acid sequence of SEQ ID NO:275; and VL4 comprises the amino acid sequence of SEQ ID NO:54. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:427; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:429. In some aspects, the first polypeptide has at least 90% identity to SEQ ID NO:427 and the second polypeptide has at least 90% identity to SEQ ID NO:429. In some aspects, the first polypeptide comprises the amino acid sequence of SEQ ID NO:427; and the second polypeptide comprises the amino acid sequence of SEQ ID NO:429. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:428; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:430. In some aspects, the first polypeptide comprises an amino acid sequence encoded by a polynucleotide having at least 90% identity to SEQ ID NO:428 and the second polypeptide comprises an amino acid sequence encoded by a polynucleotide having at least 90% identity to SEQ ID NO:430. In some aspects, the first polypeptide comprises an amino acid sequence encoded by the polynucleotide of SEQ ID NO:428; and the second polypeptide comprises an amino acid sequence encoded by the polynucleotide of SEQ ID NO:430.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH2 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VH3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:271; VL1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; VL2 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275; and VL3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:267. In some aspects, VH1 comprises the amino acid sequence of SEQ ID NO:58; VH2 and VH4 comprise the amino acid sequence of SEQ ID NO:279; VH3 comprises the amino acid sequence of SEQ ID NO:271; VL1 comprises the amino acid sequence of SEQ ID NO:54; VL2 and VL4 comprise the amino acid sequence of SEQ ID NO:275; and VL3 comprises the amino acid sequence of SEQ ID NO:267. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:431; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:433. In some aspects, the first polypeptide has at least 90% identity to SEQ ID NO:431 and the second polypeptide has at least 90% identity to SEQ ID NO:433. In some aspects, the first polypeptide comprises the amino acid sequence of SEQ ID NO:431; and the second polypeptide comprises the amino acid sequence of SEQ ID NO:433. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:432; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:434. In some aspects, the first polypeptide comprises an amino acid sequence encoded by a polynucleotide having at least 90% identity to SEQ ID NO:432 and the second polypeptide comprises an amino acid sequence encoded by a polynucleotide having at least 90% identity to SEQ ID NO:434. In some aspects, the first polypeptide comprises an amino acid sequence encoded by the polynucleotide of SEQ ID NO:432; and the second polypeptide comprises an amino acid sequence encoded by the polynucleotide of SEQ ID NO:434.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH2 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VH4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:271; VL1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; VL2 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275; and VL4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:267. In some aspects, VH1 comprises the amino acid sequence of SEQ ID NO:58; VH2 and VH3 comprise the amino acid sequence of SEQ ID NO:279; VH4 comprises the amino acid sequence of SEQ ID NO:271; VL1 comprises the amino acid sequence of SEQ ID NO:54; VL2 and VL3 comprise the amino acid sequence of SEQ ID NO:275; and VL4 comprises the amino acid sequence of SEQ ID NO:267. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:435; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:437. In some aspects, the first polypeptide has at least 90% identity to SEQ ID NO:435 and the second polypeptide has at least 90% identity to SEQ ID NO:437. In some aspects, the first polypeptide comprises the amino acid sequence of SEQ ID NO:435; and the second polypeptide comprises the amino acid sequence of SEQ ID NO:437. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:436; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:438. In some aspects, the first polypeptide comprises an amino acid sequence encoded by a polynucleotide having at least 90% identity to SEQ ID NO:436 and the second polypeptide comprises an amino acid sequence encoded by a polynucleotide having at least 90% identity to SEQ ID NO:438. In some aspects, the first polypeptide comprises an amino acid sequence encoded by the polynucleotide of SEQ ID NO:436; and the second polypeptide comprises an amino acid sequence encoded by the polynucleotide of SEQ ID NO:438.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH2 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:287; VH4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; VL2 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:283; and VL4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 comprises the amino acid sequence of SEQ ID NO:58; VH2 and VH3 comprise the amino acid sequence of SEQ ID NO:287; VH4 comprises the amino acid sequence of SEQ ID NO:279; VL1 comprises the amino acid sequence of SEQ ID NO:54; VL2 and VL3 comprise the amino acid sequence of SEQ ID NO:283; and VL4 comprises the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:439; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:441. In some aspects, the first polypeptide has at least 90% identity to SEQ ID NO:439 and the second polypeptide has at least 90% identity to SEQ ID NO:441. In some aspects, the first polypeptide comprises the amino acid sequence of SEQ ID NO:439; and the second polypeptide comprises the amino acid sequence of SEQ ID NO:441. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:440; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:442. In some aspects, the first polypeptide comprises an amino acid sequence encoded by a polynucleotide having at least 90% identity to SEQ ID NO:440 and the second polypeptide comprises an amino acid sequence encoded by a polynucleotide having at least 90% identity to SEQ ID NO:442. In some aspects, the first polypeptide comprises an amino acid sequence encoded by the polynucleotide of SEQ ID NO:440; and the second polypeptide comprises an amino acid sequence encoded by the polynucleotide of SEQ ID NO:442.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH2 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:287; VH3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; VL2 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:283; and VL3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 comprises the amino acid sequence of SEQ ID NO:58; VH2 and VH4 comprise the amino acid sequence of SEQ ID NO:287; VH3 comprises the amino acid sequence of SEQ ID NO:279; VL1 comprises the amino acid sequence of SEQ ID NO:54; VL2 and VL4 comprise the amino acid sequence of SEQ ID NO:283; and VL3 comprises the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:443; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:445. In some aspects, the first polypeptide has at least 90% identity to SEQ ID NO:443 and the second polypeptide has at least 90% identity to SEQ ID NO:445. In some aspects, the first polypeptide comprises the amino acid sequence of SEQ ID NO:443; and the second polypeptide comprises the amino acid sequence of SEQ ID NO:445. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:444; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:446. In some aspects, the first polypeptide comprises an amino acid sequence encoded by a polynucleotide having at least 90% identity to SEQ ID NO:444 and the second polypeptide comprises an amino acid sequence encoded by a polynucleotide having at least 90% identity to SEQ ID NO:446. In some aspects, the first polypeptide comprises an amino acid sequence encoded by the polynucleotide of SEQ ID NO:444; and the second polypeptide comprises an amino acid sequence encoded by the polynucleotide of SEQ ID NO:446.

In some aspects, VH1 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:287; VH2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:283; VL2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; and VL4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 and VH3 comprise the amino acid sequence of SEQ ID NO:287; VH2 comprises the amino acid sequence of SEQ ID NO:58; VH4 comprises the amino acid sequence of SEQ ID NO:279; VL1 and VL3 comprise the amino acid sequence of SEQ ID NO:283; VL2 comprises the amino acid sequence of SEQ ID NO:54; and VL4 comprises the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:447; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:449. In some aspects, the first polypeptide has at least 90% identity to SEQ ID NO:447 and the second polypeptide has at least 90% identity to SEQ ID NO:449. In some aspects, the first polypeptide comprises the amino acid sequence of SEQ ID NO:447; and the second polypeptide comprises the amino acid sequence of SEQ ID NO:449. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:448; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:450. In some aspects, the first polypeptide comprises an amino acid sequence encoded by a polynucleotide having at least 90% identity to SEQ ID NO:448 and the second polypeptide comprises an amino acid sequence encoded by a polynucleotide having at least 90% identity to SEQ ID NO:450. In some aspects, the first polypeptide comprises an amino acid sequence encoded by the polynucleotide of SEQ ID NO:448; and the second polypeptide comprises an amino acid sequence encoded by the polynucleotide of SEQ ID NO:450.

In some aspects, VH1 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:287; VH2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:283; VL2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; and VL3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 and VH4 comprise the amino acid sequence of SEQ ID NO:287; VH2 comprises the amino acid sequence of SEQ ID NO:58; VH3 comprises the amino acid sequence of SEQ ID NO:279; VL1 and VL4 comprise the amino acid sequence of SEQ ID NO:283; VL2 comprises the amino acid sequence of SEQ ID NO:54; and VL3 comprises the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:451; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:453. In some aspects, the first polypeptide has at least 90% identity to SEQ ID NO:451 and the second polypeptide has at least 90% identity to SEQ ID NO:453. In some aspects, the first polypeptide comprises the amino acid sequence of SEQ ID NO:451; and the second polypeptide comprises the amino acid sequence of SEQ ID NO:453. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:452; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:454. In some aspects, the first polypeptide comprises an amino acid sequence encoded by a polynucleotide having at least 90% identity to SEQ ID NO:452 and the second polypeptide comprises an amino acid sequence encoded by a polynucleotide having at least 90% identity to SEQ ID NO:454. In some aspects, the first polypeptide comprises an amino acid sequence encoded by the polynucleotide of SEQ ID NO:452; and the second polypeptide comprises an amino acid sequence encoded by the polynucleotide of SEQ ID NO:454.

In some aspects, VH1 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:287; VH4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; VL2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:283; and VL4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 and VH3 comprise the amino acid sequence of SEQ ID NO:58; VH2 comprises the amino acid sequence of SEQ ID NO:287; VH4 comprises the amino acid sequence of SEQ ID NO:279; VL1 and VL3 comprise the amino acid sequence of SEQ ID NO:54; VL2 comprises the amino acid sequence of SEQ ID NO:283; and VL4 comprises the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:455; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:457. In some aspects, the first polypeptide has at least 90% identity to SEQ ID NO:455 and the second polypeptide has at least 90% identity to SEQ ID NO:457. In some aspects, the first polypeptide comprises the amino acid sequence of SEQ ID NO:455; and the second polypeptide comprises the amino acid sequence of SEQ ID NO:457. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:456; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:458. In some aspects, the first polypeptide comprises an amino acid sequence encoded by a polynucleotide having at least 90% identity to SEQ ID NO:456 and the second polypeptide comprises an amino acid sequence encoded by a polynucleotide having at least 90% identity to SEQ ID NO:458. In some aspects, the first polypeptide comprises an amino acid sequence encoded by the polynucleotide of SEQ ID NO:456; and the second polypeptide comprises an amino acid sequence encoded by the polynucleotide of SEQ ID NO:458.

In some aspects, VH1 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:287; VH3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; VL2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:283; and VL3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 and VH4 comprise the amino acid sequence of SEQ ID NO:58; VH2 comprises the amino acid sequence of SEQ ID NO:287; VH3 comprises the amino acid sequence of SEQ ID NO:279; VL1 and VL4 comprise the amino acid sequence of SEQ ID NO:54; VL2 comprises the amino acid sequence of SEQ ID NO:283; and VL3 comprises the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:459; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:461. In some aspects, the first polypeptide has at least 90% identity to SEQ ID NO:459 and the second polypeptide has at least 90% identity to SEQ ID NO:461. In some aspects, the first polypeptide comprises the amino acid sequence of SEQ ID NO:459; and the second polypeptide comprises the amino acid sequence of SEQ ID NO:461. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:460; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:462. In some aspects, the first polypeptide comprises an amino acid sequence encoded by a polynucleotide having at least 90% identity to SEQ ID NO:460 and the second polypeptide comprises an amino acid sequence encoded by a polynucleotide having at least 90% identity to SEQ ID NO:462. In some aspects, the first polypeptide comprises an amino acid sequence encoded by the polynucleotide of SEQ ID NO:460; and the second polypeptide comprises an amino acid sequence encoded by the polynucleotide of SEQ ID NO:462.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:287; VH2 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:283; VL2 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; and VL4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 comprises the amino acid sequence of SEQ ID NO:287; VH2 and VH3 comprise the amino acid sequence of SEQ ID NO:58; VH4 comprises the amino acid sequence of SEQ ID NO:279; VL1 comprises the amino acid sequence of SEQ ID NO:283; VL2 and VL3 comprise the amino acid sequence of SEQ ID NO:54; and VL4 comprises the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:463; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:465. In some aspects, the first polypeptide has at least 90% identity to SEQ ID NO:463 and the second polypeptide has at least 90% identity to SEQ ID NO:465. In some aspects, the first polypeptide comprises the amino acid sequence of SEQ ID NO:463; and the second polypeptide comprises the amino acid sequence of SEQ ID NO:465. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:464; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:466. In some aspects, the first polypeptide comprises an amino acid sequence encoded by a polynucleotide having at least 90% identity to SEQ ID NO:464 and the second polypeptide comprises an amino acid sequence encoded by a polynucleotide having at least 90% identity to SEQ ID NO:466. In some aspects, the first polypeptide comprises an amino acid sequence encoded by the polynucleotide of SEQ ID NO:464; and the second polypeptide comprises an amino acid sequence encoded by the polynucleotide of SEQ ID NO:466.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:287; VH2 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:283; VL2 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; and VL3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 comprises the amino acid sequence of SEQ ID NO:287; VH2 and VH4 comprise the amino acid sequence of SEQ ID NO:58; VH3 comprises the amino acid sequence of SEQ ID NO:279; VL1 comprises the amino acid sequence of SEQ ID NO:283; VL2 and VL4 comprise the amino acid sequence of SEQ ID NO:54; and VL3 comprises the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fe effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:467; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:469. In some aspects, the first polypeptide has at least 90% identity to SEQ ID NO:467 and the second polypeptide has at least 90% identity to SEQ ID NO:469. In some aspects, the first polypeptide comprises the amino acid sequence of SEQ ID NO:467; and the second polypeptide comprises the amino acid sequence of SEQ ID NO:469. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:468; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:470. In some aspects, the first polypeptide comprises an amino acid sequence encoded by a polynucleotide having at least 90% identity to SEQ ID NO:468 and the second polypeptide comprises an amino acid sequence encoded by a polynucleotide having at least 90% identity to SEQ ID NO:470. In some aspects, the first polypeptide comprises an amino acid sequence encoded by the polynucleotide of SEQ ID NO:468; and the second polypeptide comprises an amino acid sequence encoded by the polynucleotide of SEQ ID NO:470.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:287; VH2 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VH4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VL1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:283; VL2 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275; and VL4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54. In some aspects, VH1 comprises the amino acid sequence of SEQ ID NO:287; VH2 and VH3 comprise the amino acid sequence of SEQ ID NO:279; VH4 comprises the amino acid sequence of SEQ ID NO:58; VL1 comprises the amino acid sequence of SEQ ID NO:283; VL2 and VL3 comprise the amino acid sequence of SEQ ID NO:275; and VL4 comprises the amino acid sequence of SEQ ID NO:54. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:471; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:473. In some aspects, the first polypeptide has at least 90% identity to SEQ ID NO:471 and the second polypeptide has at least 90% identity to SEQ ID NO:473. In some aspects, the first polypeptide comprises the amino acid sequence of SEQ ID NO:471; and the second polypeptide comprises the amino acid sequence of SEQ ID NO:473. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:472; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:474. In some aspects, the first polypeptide comprises an amino acid sequence encoded by a polynucleotide having at least 90% identity to SEQ ID NO:472 and the second polypeptide comprises an amino acid sequence encoded by a polynucleotide having at least 90% identity to SEQ ID NO:474. In some aspects, the first polypeptide comprises an amino acid sequence encoded by the polynucleotide of SEQ ID NO:472; and the second polypeptide comprises an amino acid sequence encoded by the polynucleotide of SEQ ID NO:474.

In some aspects, VH2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:287; VH1 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VH4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VL2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:283; VL1 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275; and VL4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54. In some aspects, VH2 comprises the amino acid sequence of SEQ ID NO:287; VH1 and VH3 comprise the amino acid sequence of SEQ ID NO:279; VH4 comprises the amino acid sequence of SEQ ID NO:58; VL2 comprises the amino acid sequence of SEQ ID NO:283; VL1 and VL3 comprise the amino acid sequence of SEQ ID NO:275; and VL4 comprises the amino acid sequence of SEQ ID NO:54. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:475; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:477. In some aspects, the first polypeptide has at least 90% identity to SEQ ID NO:475 and the second polypeptide has at least 90% identity to SEQ ID NO:477. In some aspects, the first polypeptide comprises the amino acid sequence of SEQ ID NO:475; and the second polypeptide comprises the amino acid sequence of SEQ ID NO:477. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:476; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:478. In some aspects, the first polypeptide comprises an amino acid sequence encoded by a polynucleotide having at least 90% identity to SEQ ID NO:476 and the second polypeptide comprises an amino acid sequence encoded by a polynucleotide having at least 90% identity to SEQ ID NO:478. In some aspects, the first polypeptide comprises an amino acid sequence encoded by the polynucleotide of SEQ ID NO:476; and the second polypeptide comprises an amino acid sequence encoded by the polynucleotide of SEQ ID NO:478.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH2 and VH4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VH3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:287; VL1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; VL2 and VL4 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275; and VL3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:283. In some aspects, VH1 comprises the amino acid sequence of SEQ ID NO:58; VH2 and VH4 comprise the amino acid sequence of SEQ ID NO:279; VH3 comprises the amino acid sequence of SEQ ID NO:287; VL1 comprises the amino acid sequence of SEQ ID NO:54; VL2 and VL4 comprise the amino acid sequence of SEQ ID NO:275; and VL3 comprises the amino acid sequence of SEQ ID NO:283. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:479; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:481. In some aspects, the first polypeptide has at least 90% identity to SEQ ID NO:479 and the second polypeptide has at least 90% identity to SEQ ID NO:481. In some aspects, the first polypeptide comprises the amino acid sequence of SEQ ID NO:479; and the second polypeptide comprises the amino acid sequence of SEQ ID NO:481. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:480; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:482. In some aspects, the first polypeptide comprises an amino acid sequence encoded by a polynucleotide having at least 90% identity to SEQ ID NO:480 and the second polypeptide comprises an amino acid sequence encoded by a polynucleotide having at least 90% identity to SEQ ID NO:482. In some aspects, the first polypeptide comprises an amino acid sequence encoded by the polynucleotide of SEQ ID NO:480; and the second polypeptide comprises an amino acid sequence encoded by the polynucleotide of SEQ ID NO:482.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH2 and VH3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VH4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:287; VL1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; VL2 and VL3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275; and VL4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:283. In some aspects, VH1 comprises the amino acid sequence of SEQ ID NO:58; VH2 and VH3 comprise the amino acid sequence of SEQ ID NO:279; VH4 comprises the amino acid sequence of SEQ ID NO:287; VL1 comprises the amino acid sequence of SEQ ID NO:54; VL2 and VL3 comprise the amino acid sequence of SEQ ID NO:275; and VL4 comprises the amino acid sequence of SEQ ID NO:283. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:483; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:485. In some aspects, the first polypeptide has at least 90% identity to SEQ ID NO:483 and the second polypeptide has at least 90% identity to SEQ ID NO:485. In some aspects, the first polypeptide comprises the amino acid sequence of SEQ ID NO:483; and the second polypeptide comprises the amino acid sequence of SEQ ID NO:485. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:484; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:486. In some aspects, the first polypeptide comprises an amino acid sequence encoded by a polynucleotide having at least 90% identity to SEQ ID NO:484 and the second polypeptide comprises an amino acid sequence encoded by a polynucleotide having at least 90% identity to SEQ ID NO:486. In some aspects, the first polypeptide comprises an amino acid sequence encoded by the polynucleotide of SEQ ID NO:484; and the second polypeptide comprises an amino acid sequence encoded by the polynucleotide of SEQ ID NO:486.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:271; VH3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:263; VH4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; VL2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:267; VL3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:259; and VL4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 comprises the amino acid sequence of SEQ ID NO:58; VH2 comprises the amino acid sequence of SEQ ID NO:271; VH3 comprises the amino acid sequence of SEQ ID NO:263; VH4 comprises the amino acid sequence of SEQ ID NO:279; VL1 comprises the amino acid sequence of SEQ ID NO:54; VL2 comprises the amino acid sequence of SEQ ID NO:267; VL3 comprises the amino acid sequence of SEQ ID NO:259; and VL4 comprises the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:487; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:489. In some aspects, the first polypeptide has at least 90% identity to SEQ ID NO:487 and the second polypeptide has at least 90% identity to SEQ ID NO:489. In some aspects, the first polypeptide comprises the amino acid sequence of SEQ ID NO:487; and the second polypeptide comprises the amino acid sequence of SEQ ID NO:489. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:488; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:490. In some aspects, the first polypeptide comprises an amino acid sequence encoded by a polynucleotide having at least 90% identity to SEQ ID NO:488 and the second polypeptide comprises an amino acid sequence encoded by a polynucleotide having at least 90% identity to SEQ ID NO:490. In some aspects, the first polypeptide comprises an amino acid sequence encoded by the polynucleotide of SEQ ID NO:488; and the second polypeptide comprises an amino acid sequence encoded by the polynucleotide of SEQ ID NO:490.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:287; VH3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VH4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:263; VL1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; VL2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:283; VL3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275; and VL4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:259. In some aspects, VH1 comprises the amino acid sequence of SEQ ID NO:58; VH2 comprises the amino acid sequence of SEQ ID NO:287; VH3 comprises the amino acid sequence of SEQ ID NO:279; VH4 comprises the amino acid sequence of SEQ ID NO:263; VL1 comprises the amino acid sequence of SEQ ID NO:54; VL2 comprises the amino acid sequence of SEQ ID NO:283; VL3 comprises the amino acid sequence of SEQ ID NO:275; and VL4 comprises the amino acid sequence of SEQ ID NO:259. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:491; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:493. In some aspects, the first polypeptide has at least 90% identity to SEQ ID NO:491 and the second polypeptide has at least 90% identity to SEQ ID NO:493. In some aspects, the first polypeptide comprises the amino acid sequence of SEQ ID NO:491; and the second polypeptide comprises the amino acid sequence of SEQ ID NO:493. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:492; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:494. In some aspects, the first polypeptide comprises an amino acid sequence encoded by a polynucleotide having at least 90% identity to SEQ ID NO:492 and the second polypeptide comprises an amino acid sequence encoded by a polynucleotide having at least 90% identity to SEQ ID NO:494. In some aspects, the first polypeptide comprises an amino acid sequence encoded by the polynucleotide of SEQ ID NO:492; and the second polypeptide comprises an amino acid sequence encoded by the polynucleotide of SEQ ID NO:494.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:263; VH3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:271; VH4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VL1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; VL2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:259; VL3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:267; and VL4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275. In some aspects, VH1 comprises the amino acid sequence of SEQ ID NO:58; VH2 comprises the amino acid sequence of SEQ ID NO:263; VH3 comprises the amino acid sequence of SEQ ID NO:271; VH4 comprises the amino acid sequence of SEQ ID NO:279; VL1 comprises the amino acid sequence of SEQ ID NO:54; VL2 comprises the amino acid sequence of SEQ ID NO:259; VL3 comprises the amino acid sequence of SEQ ID NO:267; and VL4 comprises the amino acid sequence of SEQ ID NO:275. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:495; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:497. In some aspects, the first polypeptide has at least 90% identity to SEQ ID NO:495 and the second polypeptide has at least 90% identity to SEQ ID NO:497. In some aspects, the first polypeptide comprises the amino acid sequence of SEQ ID NO:495; and the second polypeptide comprises the amino acid sequence of SEQ ID NO:497. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:496; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:498. In some aspects, the first polypeptide comprises an amino acid sequence encoded by a polynucleotide having at least 90% identity to SEQ ID NO:496 and the second polypeptide comprises an amino acid sequence encoded by a polynucleotide having at least 90% identity to SEQ ID NO:498. In some aspects, the first polypeptide comprises an amino acid sequence encoded by the polynucleotide of SEQ ID NO:496; and the second polypeptide comprises an amino acid sequence encoded by the polynucleotide of SEQ ID NO:498.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:263; VH3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VH4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:287; VL1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; VL2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:259; VL3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275; and VL4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:283. In some aspects, VH1 comprises the amino acid sequence of SEQ ID NO:58; VH2 comprises the amino acid sequence of SEQ ID NO:263; VH3 comprises the amino acid sequence of SEQ ID NO:279; VH4 comprises the amino acid sequence of SEQ ID NO:287; VL1 comprises the amino acid sequence of SEQ ID NO:54; VL2 comprises the amino acid sequence of SEQ ID NO:259; VL3 comprises the amino acid sequence of SEQ ID NO:275; and VL4 comprises the amino acid sequence of SEQ ID NO:283. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:499; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:501. In some aspects, the first polypeptide has at least 90% identity to SEQ ID NO:499 and the second polypeptide has at least 90% identity to SEQ ID NO:501. In some aspects, the first polypeptide comprises the amino acid sequence of SEQ ID NO:499; and the second polypeptide comprises the amino acid sequence of SEQ ID NO:501. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:500; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:502. In some aspects, the first polypeptide comprises an amino acid sequence encoded by a polynucleotide having at least 90% identity to SEQ ID NO:500 and the second polypeptide comprises an amino acid sequence encoded by a polynucleotide having at least 90% identity to SEQ ID NO:502. In some aspects, the first polypeptide comprises an amino acid sequence encoded by the polynucleotide of SEQ ID NO:500; and the second polypeptide comprises an amino acid sequence encoded by the polynucleotide of SEQ ID NO:502.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:58; VH2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:271; VH3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:279; VH4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:287; VL1 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:54; VL2 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:267; VL3 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:275; and VL4 comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:283. In some aspects, VH1 comprises the amino acid sequence of SEQ ID NO:58; VH2 comprises the amino acid sequence of SEQ ID NO:271; VH3 comprises the amino acid sequence of SEQ ID NO:279; VH4 comprises the amino acid sequence of SEQ ID NO:287; VL1 comprises the amino acid sequence of SEQ ID NO:54; VL2 comprises the amino acid sequence of SEQ ID NO:267; VL3 comprises the amino acid sequence of SEQ ID NO:275; and VL4 comprises the amino acid sequence of SEQ ID NO:283. In some aspects, L1-L16 comprise the amino acid sequence of any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG. In some aspects, L1 or L4 and L7 or L10 comprise the amino acid sequence of SEQ ID NO:6; L2 or L5 and L8 or L11 comprise the amino acid sequence of SEQ ID NO:27; and L3 or L6 and L9 or L12 comprise the amino acid sequence of SEQ ID NO:6. In some aspects, L1 or L5 and L9 or L13 comprise the amino acid sequence of SEQ ID NO:6; L2 or L6 and L10 or L14 comprise the amino acid sequence of SEQ ID NO:27; L3 or L7 and L11 or L15 comprise the amino acid sequence of SEQ ID NO:6; and L4 or L8 and L12 or L16 comprise the amino acid sequence of SEQ ID NO:12. In some aspects, Fc comprises a knob-into-hole modification or Fc effector function knockout mutation. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293 or 294. In some aspects, Fc comprises the amino acid sequence of SEQ ID NO:293 or 294. In some aspects, the sequence has a mutation at N62, for example, N62Q or N62Q at, for example, SEQ ID NO:279. In some preferred aspects, VH1, VH2, VH3, VH4, VL1, VL2, VL3 and VL4 have at least 90% identity with the corresponding amino acid sequences specified above, optionally with (i) an Fc having at least 90% identity with the corresponding amino acid sequence recited above; (ii) one or more linker sequence as specified above; and/or (iii) a mutation at N62 as recited above. In some particularly preferred aspects, VH1, VH2, VH3, VH4, VL1, VL2, VL3 and VL4 comprise or consist of the corresponding amino acid sequences specified above, optionally with (i) an Fc comprising or consisting of the corresponding amino acid sequence recited above; (ii) one or more linker sequence as specified above; and/or (iii) a mutation at N62 as recited above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:503; and the second polypeptide comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:505. In some aspects, the first polypeptide has at least 90% identity to SEQ ID NO:503 and the second polypeptide has at least 90% identity to SEQ ID NO:505. In some aspects, the first polypeptide comprises the amino acid sequence of SEQ ID NO:503; and the second polypeptide comprises the amino acid sequence of SEQ ID NO:505. In some aspects, the first polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:504; and the second polypeptide comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to SEQ ID NO:506. In some aspects, the first polypeptide comprises an amino acid sequence encoded by a polynucleotide having at least 90% identity to SEQ ID NO:504 and the second polypeptide comprises an amino acid sequence encoded by a polynucleotide having at least 90% identity to SEQ ID NO:506. In some aspects, the first polypeptide comprises an amino acid sequence encoded by the polynucleotide of SEQ ID NO:504; and the second polypeptide comprises an amino acid sequence encoded by the polynucleotide of SEQ ID NO:506.

In some aspects, an antigen binding polypeptide provided herein is part of a hexavalent antigen binding polypeptide complex. Such polypeptides can have any VH, VL, CDR, CH1, CL, Fc or amino acid linker sequence disclosed herein. For example, provided herein is an antigen binding polypeptide having a structure represented by VL1-VL2-VL3-VH3-VH2-VH1; VH1-VH2-VH3-VL3-VL2-VL1; VL1-VH2-VL3-VH3-VL2-VH1; VH1-VL2-VH3-VL3-VH2-VL1; VL1-VL2-VH3-VL3-VH2-VH1; VH1-VH2-VL3-VH3-VL2-VL1; VL1-VH2-VH3-VL3-VL2-VH1; VH1-VL2-VL3-VH3-VH2-VL1; VL1-L1-VL2-L2-VL3-L3-VH3-L4-VH2-L5-VH1; VH1-L1-VH2-L2-VH3-L3-VL3-L4-VL2-L5-VL1; VL1-L1-VH2-L2-VL3-L3-VH3-L4-VL2-L5-VH1; VH1-L1-VL2-L2-VH3-L3-VL3-L4-VH2-L5-VL1; VL1-L1-VL2-L2-VH3-L3-VL3-L4-VH2-L5-VH1; VH1-L1-VH2-L2-VL3-L3-VH3-L4-VL2-L5-VL1; VL1-L1-VH2-L2-VH3-L3-VL3-L4-VL2-L5-VH1; or VH1-L1-VL2-L2-VL3-L3-VH3-L4-VH2-L5-VL1; wherein VL11 is a first immunoglobulin light chain variable region; VL2 is a second immunoglobulin light chain variable region; VL3 is a third immunoglobulin light chain variable region; VH1 is a first immunoglobulin heavy chain variable region; VH2 is a second immunoglobulin heavy chain variable region; VH3 is a third immunoglobulin heavy chain variable region; and L1, L2, L3, L4 and L5 are amino acid linkers.

In some aspects, an antigen binding polypeptide complex provided herein is hexavalent. Such complexes can have any VH, VL, CDR, CH1, CL, Fc or amino acid linker sequence disclosed herein. For example, an antigen binding polypeptide complex can comprise a first polypeptide and a second polypeptide; wherein the first polypeptide has a structure represented by VL1-VL2-VL3-VH3-VH2-VH1; VH1-VH2-VH3-VL3-VL2-VL1; VL1-VH2-VL3-VH3-VL2-VH1; VH1-VL2-VH3-VL3-VH2-VL1; VL1-VL2-VH3-VL3-VH2-VH1; VH1-VH2-VL3-VH3-VL2-VL1; VL1-VH2-VH3-VL3-VL2-VH1; VH1-VL2-VL3-VH3-VH2-VL1; VL1-L1-VL2-L2-VL3-L3-VH3-L4-VH2-L5-VH1; VH1-L1-VH2-L2-VH3-L3-VL3-L4-VL2-L5-VL1; VL1-L1-VH2-L2-VL3-L3-VH3-L4-VL2-L5-VH1; VH1-L1-VL2-L2-VH3-L3-VL3-L4-VH2-L5-VL1; VL1-L1-VL2-L2-VH3-L3-VL3-L4-VH2-L5-VH1; VH1-L1-VH2-L2-VL3-L3-VH3-L4-VL2-L5-VL1; VL1-L1-VH2-L2-VH3-L3-VL3-L4-VL2-L5-VH1; or VH1-L1-VL2-L2-VL3-L3-VH3-L4-VH2-L5-VL1; wherein the second polypeptide has a structure represented by VL4-VH4; VH4-VL4; VL4-L6-VH4; VH4-L6-VL4; VL4-VL5-VH5-VH4; VH4-VH5-VL5-VL4; VL4-L6-VL5-L7-VH5-L8-VH4; VH4-L6-VH5-L7-VL5-L8-VL4; VL4-VL5-VL6-VH6-VH5-VH4; VH4-VH5-VH6-VL6-VL5-VL4; VL4-VH5-VL6-VH6-VL5-VH4; VH4-VL5-VH6-VL6-VH5-VL4; VL4-VL5-VH6-VL6-VH5-VH4; VH4-VH5-VL6-VH6-VL5-VL4; VL4-VH5-VH6-VL6-VL5-VH4; VH4-VL5-VL6-VH6-VH5-VL4; VL4-L6-VL5-L7-VL6-L8-VH6-L9-VH5-L10-VH4; VH4-L6-VH5-L7-VH6-L8-VL6-L9-VL5-L10-VL4; VL4-L6-VH5-L7-VL6-L8-VH6-L9-VL5-L10-VH4; VH4-L6-VL5-L7-VH6-L8-VL6-L9-VH5-L10-VL4; VL4-L6-VL5-L7-VH6-L8-VL6-L9-VH5-L10-VH4; VH4-L6-VH5-L7-VL6-L8-VH6-L9-VL5-L10-VL4; VL4-L6-VH5-L7-VH6-L8-VL6-L9-VL5-L10-VH4; or VH4-L6-VL5-L7-VL6-L8-VH6-L9-VH5-L10-VL4; wherein VL1 is a first immunoglobulin light chain variable region; VL2 is a second immunoglobulin light chain variable region; VL3 is a third immunoglobulin light chain variable region; VL4 is a fourth immunoglobulin light chain variable region; VL5 is a fifth immunoglobulin light chain variable region; VL6 is a sixth immunoglobulin light chain variable region; VH1 is a first immunoglobulin heavy chain variable region; VH2 is a second immunoglobulin heavy chain variable region; VH3 is a third immunoglobulin heavy chain variable region; VH4 is a fourth immunoglobulin heavy chain variable region; VH5 is a fifth immunoglobulin heavy chain variable region; VH6 is a sixth immunoglobulin heavy chain variable region; and L1, L2, L3, L4, L5, L6, L7, L8, L9 and L10 are amino acid linkers. Any combination of VH1, VH2, VH3 and/or VH4 described above may be used in combination with any combination of VL1, VL2, VL3 and/or VL4 described above. Thus, any first polypeptide described above may be used in any combination with any second polypeptide described above.

In some aspects, an antigen binding polypeptide provided herein has a structure represented by VL1-VL2-VL3-VH3-VH2-VH1-Fc; VH1-VH2-VH3-VL3-VL2-VL1-Fc; VL1-VH2-VL3-VH3-VL2-VH1-Fc; VH1-VL2-VH3-VL3-VH2-VL1-Fc; VL1-VL2-VH3-VL3-VH2-VH1-Fc; VH1-VH2-VL3-VH3-VL2-VL1-Fc; VL1-VH2-VH3-VL3-VL2-VH1-Fc; VH1-VL2-VL3-VH3-VH2-VL1-Fc; VL1-L1-VL2-L2-VL3-L3-VH3-L4-VH2-L5-VH1-Fc; VL1-L1-VL2-L2-VL3-L3-VH3-L4-VH2-L5-VH1-L6-Fc; VH1-L1-VH2-L2-VH3-L3-VL3-L4-VL2-L5-VL1-Fc; VH1-L1-VH2-L2-VH3-L3-VL3-L4-VL2-L5-VL1-L6-Fc; VL1-L1-VH2-L2-VL3-L3-VH3-L4-VL2-L5-VH1-Fc; VL1-L1-VH2-L2-VL3-L3-VH3-L4-VL2-L5-VH1-L6-Fc; VH1-L1-VL2-L2-VH3-L3-VL3-L4-VH2-L5-VL1-Fc; VH1-L1-VL2-L2-VH3-L3-VL3-L4-VH2-L5-VL1-L6-Fc; VL1-L1-VL2-L2-VH3-L3-VL3-L4-VH2-L5-VH1-Fc; VL1-L1-VL2-L2-VH3-L3-VL3-L4-VH2-L5-VH1-L6-Fc; VH1-L1-VH2-L2-VL3-L3-VH3-L4-VL2-L5-VL1-Fc; VH1-L1-VH2-L2-VL3-L3-VH3-L4-VL2-L5-VL1-L6-Fc; VL1-L1-VH2-L2-VH3-L3-VL3-L4-VL2-L5-VH1-Fc; VL1-L1-VH2-L2-VH3-L3-VL3-L4-VL2-L5-VH1-L6-Fc; VH1-L1-VL2-L2-VL3-L3-VH3-L4-VH2-L5-VL1-Fc; or VH1-L1-VL2-L2-VL3-L3-VH3-L4-VH2-L5-VL1-L6-Fc; wherein VL1 is a first immunoglobulin light chain variable region; VL2 is a second immunoglobulin light chain variable region; VL3 is a third immunoglobulin light chain variable region; VH1 is a first immunoglobulin heavy chain variable region; VH2 is a second immunoglobulin heavy chain variable region; VH3 is a third immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5 and L6 are amino acid linkers.

In some aspects, an antigen binding polypeptide provided herein has a structure represented by VL1-VL2-VL3-VH3-VH2-VH1-Fc-Fc; VH1-VH2-VH3-VL3-VL2-VL1-Fc-Fc; VL1-VH2-VL3-VH3-VL2-VH1-Fc-Fc; VH1-VL2-VH3-VL3-VH2-VL1-Fc-Fc; VL1-VL2-VH3-VL3-VH2-VH1-Fc-Fc; VH1-VH2-VL3-VH3-VL2-VL1-Fc-Fc; VL1-VH2-VH3-VL3-VL2-VH1-Fc-Fc; VH1-VL2-VL3-VH3-VH2-VL1-Fc-Fc; VL1-L1-VL2-L2-VL3-L3-VH3-L4-VH2-L5-VH1-Fc-Fc; VL1-L1-VL2-L2-VL3-L3-VH3-L4-VH2-L5-VH1-L6-Fc-Fc; VL1-L1-VL2-L2-VL3-L3-VH3-L4-VH2-L5-VH1-L6-Fc-L7-Fc; VH1-L1-VH2-L2-VH3-L3-VL3-L4-VL2-L5-VL1-Fc-Fc; VH1-L1-VH2-L2-VH3-L3-VL3-L4-VL2-L5-VL1-L6-Fc-Fc; VH1-L1-VH2-L2-VH3-L3-VL3-L4-VL2-L5-VL1-L6-Fc-L7-Fc; VL1-L1-VH2-L2-VL3-L3-VH3-L4-VL2-L5-VH1-Fc-Fc; VL1-L1-VH2-L2-VL3-L3-VH3-L4-VL2-L5-VH1-L6-Fc-Fc; VL1-L1-VH2-L2-VL3-L3-VH3-L4-VL2-L5-VH1-L6-Fc-L7-Fc; VH1-L1-VL2-L2-VH3-L3-VL3-L4-VH2-L5-VL1-Fc-Fc; VH1-L1-VL2-L2-VH3-L3-VL3-L4-VH2-L5-VL1-L6-Fc-Fc; VH1-L1-VL2-L2-VH3-L3-VL3-L4-VH2-L5-VL1-L6-Fc-L7-Fc; VL1-L1-VL2-L2-VH3-L3-VL3-L4-VH2-L5-VH1-Fc-Fc; VL1-L1-VL2-L2-VH3-L3-VL3-L4-VH2-L5-VH1-L6-Fc-Fc; VL1-L1-VL2-L2-VH3-L3-VL3-L4-VH2-L5-VH1-L6-Fc-L7-Fc; VH1-L1-VH2-L2-VL3-L3-VH3-L4-VL2-L5-VL1-Fc-Fc; VH1-L1-VH2-L2-VL3-L3-VH3-L4-VL2-L5-VL1-L6-Fc-Fc; VH1-L1-VH2-L2-VL3-L3-VH3-L4-VL2-L5-VL1-L6-Fc-L7-Fc; L1-L1-VH2-L2-VH3-L3-VL3-L4-VL2-L5-VH1-Fc-Fc; VL1-L1-VH2-L2-VH3-L3-VL3-L4-VL2-L5-VH1-L6-Fc-Fc; VL1-L1-VH2-L2-VH3-L3-VL3-L4-VL2-L5-VH1-L6-Fc-L7-Fc; VH1-L1-VL2-L2-VL3-L3-VH3-L4-VH2-L5-VL1-Fc-Fc; VH1-L1-VL2-L2-VL3-L3-VH3-L4-VH2-L5-VL1-L6-Fc-Fc; or VH1-L1-VL2-L2-VL3-L3-VH3-L4-VH2-L5-VL1-L6-Fc-L7-Fc; wherein VL1 is a first immunoglobulin light chain variable region; VL2 is a second immunoglobulin light chain variable region; VL3 is a third immunoglobulin light chain variable region; VH1 is a first immunoglobulin heavy chain variable region; VH2 is a second immunoglobulin heavy chain variable region; VH3 is a third immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5, L6 and L7 are amino acid linkers.

In some aspects, an antigen binding polypeptide complex provided herein comprises a first polypeptide and a second polypeptide; wherein the first polypeptide has a structure represented by VL1-VL2-VL3-VH3-VH2-VH1-Fc; VH1-VH2-VH3-VL3-VL2-VL1-Fc; VL1-VH2-VL3-VH3-VL2-VH1-Fc; VH1-VL2-VH3-VL3-VH2-VL1-Fc; VL1-VL2-VH3-VL3-VH2-VH1-Fc; VH1-VH2-VL3-VH3-VL2-VL1-Fc; VL1-VH2-VH3-VL3-VL2-VH1-Fc; VH1-VL2-VL3-VH3-VH2-VL1-Fc; VL1-L1-VL2-L2-VL3-L3-VH3-L4-VH2-L5-VH1-Fc; VL1-L1-VL2-L2-VL3-L3-VH3-L4-VH2-L5-VH1-L6-Fc; VH1-L1-VH2-L2-VH3-L3-VL3-L4-VL2-L5-VL1-Fc; VH1-L1-VH2-L2-VH3-L3-VL3-L4-VL2-L5-VL1-L6-Fc; VL1-L1-VH2-L2-VL3-L3-VH3-L4-VL2-L5-VH1-Fc; VL1-L1-VH2-L2-VL3-L3-VH3-L4-VL2-L5-VH1-L6-Fc; VH1-L1-VL2-L2-VH3-L3-VL3-L4-VH2-L5-VL1-Fc; VH1-L1-VL2-L2-VH3-L3-VL3-L4-VH2-L5-VL1-L6-Fc; VL1-L1-VL2-L2-VH3-L3-VL3-L4-VH2-L5-VH1-Fc; VL1-L1-VL2-L2-VH3-L3-VL3-L4-VH2-L5-VH1-L6-Fc; VH1-L1-VH2-L2-VL3-L3-VH3-L4-VL2-L5-VL1-Fc; VH1-L1-VH2-L2-VL3-L3-VH3-L4-VL2-L5-VL1-L6-Fc; VL1-L1-VH2-L2-VH3-L3-VL3-L4-VL2-L5-VH1-Fc; VL1-L1-VH2-L2-VH3-L3-VL3-L4-VL2-L5-VH1-L6-Fc; VH1-L1-VL2-L2-VL3-L3-VH3-L4-VH2-L5-VL1-Fc; or VH1-L1-VL2-L2-VL3-L3-VH3-L4-VH2-L5-VL1-L6-Fc; wherein the second polypeptide has a structure represented by Fc; VL4-VH4-Fc; VH4-VL4-Fc; VL4-L7-VH4-Fc; VH4-L7-VL4-Fc; VL4-L7-VH4-L8-Fc; VH4-L7-VL4-L8-Fc; VL4-CL-VH4-CH1-Fc; VH4-CL-VL4-CH1-Fc; VL4-CH1-VH4-CL-Fc; VH4-CH1-VL4-CL-Fc; VL4-L7-CL-L8-VH4-L9-CH1-Fc; VL4-L7-CL-L8-VH4-L9-CH1-L10-Fc; VH4-L7-CL-L8-VL4-L9-CH1-Fc; VH4-L7-CL-L8-VL4-L9-CH1-L10-Fc; VL4-L7-CH1-L8-VH4-L9-CL-Fc; VL4-L7-CH1-L8-VH4-L9-CL-L10-Fc; VH4-L7-CH1-L8-VL4-L9-CL-Fc; VH4-L7-CH1-L8-VL4-L9-CL-L10-Fc; VL4-VL5-VH5-VH4-Fc; VH4-VH5-VL5-VL4-Fc; VL4-L7-VL5-L8-VH5-L9-VH4-Fc; VH4-L7-VH5-L8-VL5-L9-VL4-Fc; VL4-L7-VL5-L8-VH5-L9-VH4-L10-Fc; VH4-L7-VH5-L8-VL5-L9-VL4-L10-Fc; VL4-VL5-VL6-VH6-VH5-VH4-Fc; VH4-VH5-VH6-VL6-VL5-VL4-Fc; VL4-VH5-VL6-VH6-VL5-VH4-Fc; VH4-VL5-VH6-VL6-VH5-VL4-Fc; VL4-VL5-VH6-VL6-VH5-VH4-Fc; VH4-VH5-VL6-VH6-VL5-VL4-Fc; VL4-VH5-VH6-VL6-VL5-VH4-Fc; VH4-VL5-VL6-VH6-VH5-VL4-Fc; VL4-L7-VL5-L8-VL6-L9-VH6-L10-VH5-L11-VH4-Fc; VH4-L7-VH5-L8-VH6-L9-VL6-L10-VL5-L11-VL4-Fc; VL4-L7-VH5-L8-VL6-L9-VH6-L10-VL5-L11-VH4-Fc; VH4-L7-VL5-L8-VH6-L9-VL6-L10-VH5-L11-VL4-Fc; VL4-L7-VL5-L8-VH6-L9-VL6-L10-VH5-L11-VH4-Fc; VH4-L7-VH5-L8-VL6-L9-VH6-L10-VL5-L11-VL4-Fc; VL4-L7-VH5-L8-VH6-L9-VL6-L10-VL5-L11-VH4-Fc; VH4-L7-VL5-L8-VL6-L9-VH6-L10-VH5-L11-VL4-Fc; VL4-L7-VL5-L8-VL6-L9-VH6-L10-VH5-L11-VH4-L12-Fc; VH4-L7-VH5-L8-VH6-L9-VL6-L10-VL5-L11-VL4-L12-Fc; VL4-L7-VH5-L8-VL6-L9-VH6-L10-VL5-L11-VH4-L12-Fc; VH4-L7-VL5-L8-VH6-L9-VL6-L10-VH5-L11-VL4-L12-Fc; VL4-L7-VL5-L8-VH6-L9-VL6-L10-VH5-L11-VH4-L12-Fc; VH4-L7-VH5-L8-VL6-L9-VH6-L10-VL5-L11-VL4-L12-Fc; VL4-L7-VH5-L8-VH6-L9-VL6-L10-VL5-L11-VH4-L12-Fc; or VH4-L7-VL5-L8-VL6-L9-VH6-L10-VH5-L11-VL4-L12-Fc; wherein VL1 is a first immunoglobulin light chain variable region; VL2 is a second immunoglobulin light chain variable region; VL3 is a third immunoglobulin light chain variable region; VL4 is a fourth immunoglobulin light chain variable region; VL5 is a fifth immunoglobulin light chain variable region; VL6 is a sixth immunoglobulin light chain variable region; VH1 is a first immunoglobulin heavy chain variable region; VH2 is a second immunoglobulin heavy chain variable region; VH3 is a third immunoglobulin heavy chain variable region; VH4 is a fourth immunoglobulin heavy chain variable region; VH5 is a fifth immunoglobulin heavy chain variable region; VH6 is a sixth immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; CH1 is a heavy chain constant region 1; CL is a light chain constant region; and L1, L2, L3, L4, L5, L6, L7, L8, L9, L10, L11 and L12 are amino acid linkers. Any combination of VH1, VH2, VH3 and/or VH4 described above may be used in combination with any combination of VL1, VL2, VL3 and/or VL4 described above. Thus, any first polypeptide described above may be used in any combination with any second polypeptide described above.

In some aspects, an antigen binding polypeptide provided herein comprises a structure represented by VL1-VL2-VL3-VH3-VH2-VH1-CH1-CL; VL1-VL2-VL3-VH3-VH2-VH1-CL-CH1; VH1-VH2-VH3-VL3-VL2-VL1-CH1-CL; VH1-VH2-VH3-VL3-VL2-VL1-CL-CH1; VL1-VH2-VL3-VH3-VL2-VH1-CH1-CL; VL1-VH2-VL3-VH3-VL2-VH1-CL-CH1; VH1-VL2-VH3-VL3-VH2-VL1-CH1-CL; VH1-VL2-VH3-VL3-VH2-VL1-CL-CH1; VL1-VL2-VH3-VL3-VH2-VH1-CH1-CL; VL1-VL2-VH3-VL3-VH2-VH1-CL-CH1; VH1-VH2-VL3-VH3-VL2-VL1-CH1-CL; VH1-VH2-VL3-VH3-VL2-VL1-CL-CH1; VL1-VH2-VH3-VL3-VL2-VH1-CH1-CL; VL1-VH2-VH3-VL3-VL2-VH1-CL-CH1; VH1-VL2-VL3-VH3-VH2-VL1-CH1-CL; VH1-VL2-VL3-VH3-VH2-VL1-CL-CH1; VL1-L1-VL2-L2-VL3-L3-VH3-L4-VH2-L5-VH1-CH1-CL; VL1-L1-VL2-L2-VL3-L3-VH3-L4-VH2-L5-VH1-L6-CH1-CL; VL1-L1-VL2-L2-VL3-L3-VH3-L4-VH2-L5-VH1-CL-CH1; VL1-L1-VL2-L2-VL3-L3-VH3-L4-VH2-L5-VH1-L6-CL-CH1; VL1-L1-VL2-L2-VL3-L3-VH3-L4-VH2-L5-VH1-L6-CL-L7-CH1; VH1-L1-VH2-L2-VH3-L3-VL3-L4-VL2-L5-VL1-CH1-CL; VH1-L1-VH2-L2-VH3-L3-VL3-L4-VL2-L5-VL1-L6-CH1-CL; VH1-L1-VH2-L2-VH3-L3-VL3-L4-VL2-L5-VL1-L6-CH1-L7-CL; VH1-L1-VH2-L2-VH3-L3-VL3-L4-VL2-L5-VL1-CL-CH1; VH1-L1-VH2-L2-VH3-L3-VL3-L4-VL2-L5-VL1-L6-CL-CH1; VL1-L1-VH2-L2-VL3-L3-VH3-L4-VL2-L5-VH1-CH1-CL; VL1-L1-VH2-L2-VL3-L3-VH3-L4-VL2-L5-VH1-L6-CH1-CL; VL1-L1-VH2-L2-VL3-L3-VH3-L4-VL2-L5-VH1-L6-CH1-L7-CL; VL1-L1-VH2-L2-VL3-L3-VH3-L4-VL2-L5-VH1-CL-CH1; VL1-L1-VH2-L2-VL3-L3-VH3-L4-VL2-L5-VH1-L6-CL-CH1; VH1-L1-VL2-L2-VH3-L3-VL3-L4-VH2-L5-VL1-CH1-CL; VH1-L1-VL2-L2-VH3-L3-VL3-L4-VH2-L5-VL1-L6-CH1-CL; VH1-L1-VL2-L2-VH3-L3-VL3-L4-VH2-L5-VL1-L6-CH1-L7-CL; VH1-L1-VL2-L2-VH3-L3-VL3-L4-VH2-L5-VL1-CL-CH1; VH1-L1-VL2-L2-VH3-L3-VL3-L4-VH2-L5-VL1-L6-CL-CH1; VH1-L1-VL2-L2-VH3-L3-VL3-L4-VH2-L5-VL1-L6-CL-L7-CH1; VL1-L1-VL2-L2-VH3-L3-VL3-L4-VH2-L5-VH1-CH1-CL; VL1-L1-VL2-L2-VH3-L3-VL3-L4-VH2-L5-VH1-L6-CH1-CL; VL1-L1-VL2-L2-VH3-L3-VL3-L4-VH2-L5-VH1-L6-CH1-L7-CL; VL1-L1-VL2-L2-VH3-L3-VL3-L4-VH2-L5-VH1-CL-CH1; VL1-L1-VL2-L2-VH3-L3-VL3-L4-VH2-L5-VH1-L6-CL-CH1; VL1-L1-VL2-L2-VH3-L3-VL3-L4-VH2-L5-VH1-L6-CL-L7-CH1; VH1-L1-VH2-L2-VL3-L3-VH3-L4-VL2-L5-VL1-CH1-CL; VH1-L1-VH2-L2-VL3-L3-VH3-L4-VL2-L5-VL1-L6-CH1-CL; VH1-L1-VH2-L2-VL3-L3-VH3-L4-VL2-L5-VL1-L6-CH1-L7-CL; VH1-L1-VH2-L2-VL3-L3-VH3-L4-VL2-L5-VL1-CL-CH1; VH1-L1-VH2-L2-VL3-L3-VH3-L4-VL2-L5-VL1-L6-CL-CH1; VH1-L1-VH2-L2-VL3-L3-VH3-L4-VL2-L5-VL1-L6-CL-L7-CH1; VL1-L1-VH2-L2-VH3-L3-VL3-L4-VL2-L5-VH1-L6-CL; VL1-L1-VH2-L2-VH3-L3-VL3-L4-VL2-L5-VH1-CH1-CL; VL1-L1-VH2-L2-VH3-L3-VL3-L4-VL2-L5-VH1-L6-CH1-CL; VL1-L1-VH2-L2-VH3-L3-VL3-L4-VL2-L5-VH1-L6-CH1-L7-CL; VL1-L1-VH2-L2-VH3-L3-VL3-L4-VL2-L5-VH1-CL-CH1; VL1-L1-VH2-L2-VH3-L3-VL3-L4-VL2-L5-VH1-L6-CL-CH1; VL1-L1-VH2-L2-VH3-L3-VL3-L4-VL2-L5-VH1-L6-CL-L7-CH1; VH1-L1-VL2-L2-VL3-L3-VH3-L4-VH2-L5-VL1-CH1-CL; VH1-L1-VL2-L2-VL3-L3-VH3-L4-VH2-L5-VL1-L6-CH1-CL; VH1-L1-VL2-L2-VL3-L3-VH3-L4-VH2-L5-VL1-L6-CH1-L7-CL; VH1-L1-VL2-L2-VL3-L3-VH3-L4-VH2-L5-VL1-CL-CH1; VH1-L1-VL2-L2-VL3-L3-VH3-L4-VH2-L5-VL1-L6-CL-CH1; or VH1-L1-VL2-L2-VL3-L3-VH3-L4-VH2-L5-VL1-L6-CL-L7-CH1; wherein VL1 is a first immunoglobulin light chain variable region; VL2 is a second immunoglobulin light chain variable region; VL3 is a third immunoglobulin light chain variable region; VH1 is a first immunoglobulin heavy chain variable region; VH2 is a second immunoglobulin heavy chain variable region; VH3 is a third immunoglobulin heavy chain variable region; CH1 is a heavy chain constant region 1; CL is a light chain constant region; and L1, L2, L3, L4, L5, L6 and L7 are amino acid linkers.

In some aspects, an antigen binding polypeptide complex provided herein comprises a first polypeptide and a second polypeptide; wherein the first polypeptide has a structure represented by VL1-VL2-VL3-VH3-VH2-VH1-CH1; VL1-VL2-VL3-VH3-VH2-VH1-CL; VL1-VL2-VL3-VH3-VH2-VH1-CH1-CL; VL1-VL2-VL3-VH3-VH2-VH1-CL-CH1; VH1-VH2-VH3-VL3-VL2-VL1-CH1; VH1-VH2-VH3-VL3-VL2-VL1-CL; VH1-VH2-VH3-VL3-VL2-VL1-CH1-CL; VH1-VH2-VH3-VL3-VL2-VL1-CL-CH1; VL1-VH2-VL3-VH3-VL2-VH1-CH1; VL1-VH2-VL3-VH3-VL2-VH1-CL; VL1-VH2-VL3-VH3-VL2-VH1-CH1-CL; VL1-VH2-VL3-VH3-VL2-VH1-CL-CH1; VH1-VL2-VH3-VL3-VH2-VL1-CH1; VH1-VL2-VH3-VL3-VH2-VL1-CL; VH1-VL2-VH3-VL3-VH2-VL1-CH1-CL; VH1-VL2-VH3-VL3-VH2-VL1-CL-CH1; VL1-VL2-VH3-VL3-VH2-VH1-CH1; VL1-VL2-VH3-VL3-VH2-VH1-CL; VL1-VL2-VH3-VL3-VH2-VH1-CH1-CL; VL1-VL2-VH3-VL3-VH2-VH1-CL-CH1; VH1-VH2-VL3-VH3-VL2-VL1-CH1; VH1-VH2-VL3-VH3-VL2-VL1-CL; VH1-VH2-VL3-VH3-VL2-VL1-CH1-CL; VH1-VH2-VL3-VH3-VL2-VL1-CL-CH1; VL1-VH2-VH3-VL3-VL2-VH1-CH1; VL1-VH2-VH3-VL3-VL2-VH1-CL; VL1-VH2-VH3-VL3-VL2-VH1-CH1-CL; VL1-VH2-VH3-VL3-VL2-VH1-CL-CH1; VH1-VL2-VL3-VH3-VH2-VL1-CH1; VH1-VL2-VL3-VH3-VH2-VL1-CL; VH1-VL2-VL3-VH3-VH2-VL1-CH1-CL; VH1-VL2-VL3-VH3-VH2-VL1-CL-CH1; VL1-L1-VL2-L2-VL3-L3-VH3-L4-VH2-L5-VH1-CH1; VL1-L1-VL2-L2-VL3-L3-VH3-L4-VH2-L5-VH1-L6-CH1; VL1-L1-VL2-L2-VL3-L3-VH3-L4-VH2-L5-VH1-CL; VL1-L1-VL2-L2-VL3-L3-VH3-L4-VH2-L5-VH1-L6-CL; VL1-L1-VL2-L2-VL3-L3-VH3-L4-VH2-L5-VH1-CH1-CL; VL1-L1-VL2-L2-VL3-L3-VH3-L4-VH2-L5-VH1-L6-CH1-CL; VL1-L1-VL2-L2-VL3-L3-VH3-L4-VH2-L5-VH1-L6-CH1-L7-CL; VL1-L1-VL2-L2-VL3-L3-VH3-L4-VH2-L5-VH1-CL-CH1; VL1-L1-VL2-L2-VL3-L3-VH3-L4-VH2-L5-VH1-L6-CL-CH1; VL1-L1-VL2-L2-VL3-L3-VH3-L4-VH2-L5-VH1-L6-CL-L7-CH1; VH1-L1-VH2-L2-VH3-L3-VL3-L4-VL2-L5-VL1-CH1; VH1-L1-VH2-L2-VH3-L3-VL3-L4-VL2-L5-VL1-L6-CH1; VH1-L1-VH2-L2-VH3-L3-VL3-L4-VL2-L5-VL1-CL; VH1-L1-VH2-L2-VH3-L3-VL3-L4-VL2-L5-VL1-L6-CL; VH1-L1-VH2-L2-VH3-L3-VL3-L4-VL2-L5-VL1-CH1-CL; VH1-L1-VH2-L2-VH3-L3-VL3-L4-VL2-L5-VL1-L6-CH1-CL; VH1-L1-VH2-L2-VH3-L3-VL3-L4-VL2-L5-VL1-L6-CH1-L7-CL; VH1-L1-VH2-L2-VH3-L3-VL3-L4-VL2-L5-VL1-CL-CH1; VH1-L1-VH2-L2-VH3-L3-VL3-L4-VL2-L5-VL1-L6-CL-CH1; VH1-L1-VH2-L2-VH3-L3-VL3-L4-VL2-L5-VL1-L6-CL-L7-CH1; VL1-L1-VH2-L2-VL3-L3-VH3-L4-VL2-L5-VH1-CH1; VL1-L1-VH2-L2-VL3-L3-VH3-L4-VL2-L5-VH1-L6-CH1; VL1-L1-VH2-L2-VL3-L3-VH3-L4-VL2-L5-VH1-CL; VL1-L1-VH2-L2-VL3-L3-VH3-L4-VL2-L5-VH1-L6-CL; VL1-L1-VH2-L2-VL3-L3-VH3-L4-VL2-L5-VH1-CH1-CL; VL1-L1-VH2-L2-VL3-L3-VH3-L4-VL2-L5-VH1-L6-CH1-CL; VL1-L1-VH2-L2-VL3-L3-VH3-L4-VL2-L5-VH1-L6-CH1-L7-CL; VL1-L1-VH2-L2-VL3-L3-VH3-L4-VL2-L5-VH1-CL-CH1; VL1-L1-VH2-L2-VL3-L3-VH3-L4-VL2-L5-VH1-L6-CL-CH1; VL1-L1-VH2-L2-VL3-L3-VH3-L4-VL2-L5-VH1-L6-CL-L7-CH1; VH1-L1-VL2-L2-VH3-L3-VL3-L4-VH2-L5-VL1-CH1; VH1-L1-VL2-L2-VH3-L3-VL3-L4-VH2-L5-VL1-L6-CH1; VH1-L1-VL2-L2-VH3-L3-VL3-L4-VH2-L5-VL1-CL; VH1-L1-VL2-L2-VH3-L3-VL3-L4-VH2-L5-VL1-L6-CL; VH1-L1-VL2-L2-VH3-L3-VL3-L4-VH2-L5-VL1-CH1-CL; VH1-L1-VL2-L2-VH3-L3-VL3-L4-VH2-L5-VL1-L6-CH1-CL; VH1-L1-VL2-L2-VH3-L3-VL3-L4-VH2-L5-VL1-L6-CH1-L7-CL; VH1-L1-VL2-L2-VH3-L3-VL3-L4-VH2-L5-VL1-CL-CH1; VH1-L1-VL2-L2-VH3-L3-VL3-L4-VH2-L5-VL1-L6-CL-CH1; VH1-L1-VL2-L2-VH3-L3-VL3-L4-VH2-L5-VL1-L6-CL-L7-CH1; VL1-L1-VL2-L2-VH3-L3-VL3-L4-VH2-L5-VH1-CH1; VL1-L1-VL2-L2-VH3-L3-VL3-L4-VH2-L5-VH1-L6-CH1; VL1-L1-VL2-L2-VH3-L3-VL3-L4-VH2-L5-VH1-CL; VL1-L1-VL2-L2-VH3-L3-VL3-L4-VH2-L5-VH1-L6-CL; VL1-L1-VL2-L2-VH3-L3-VL3-L4-VH2-L5-VH1-CH1-CL; VL1-L1-VL2-L2-VH3-L3-VL3-L4-VH2-L5-VH1-L6-CH1-CL; VL1-L1-VL2-L2-VH3-L3-VL3-L4-VH2-L5-VH1-L6-CH1-L7-CL; VL1-L1-VL2-L2-VH3-L3-VL3-L4-VH2-L5-VH1-CL-CH1; VL1-L1-VL2-L2-VH3-L3-VL3-L4-VH2-L5-VH1-L6-CL-CH1; VL1-L1-VL2-L2-VH3-L3-VL3-L4-VH2-L5-VH1-L6-CL-L7-CH1; VH1-L1-VH2-L2-VL3-L3-VH3-L4-VL2-L5-VL1-CH1; VH1-L1-VH2-L2-VL3-L3-VH3-L4-VL2-L5-VL1-L6-CH1; VH1-L1-VH2-L2-VL3-L3-VH3-L4-VL2-L5-VL1-CL; VH1-L1-VH2-L2-VL3-L3-VH3-L4-VL2-L5-VL1-L6-CL; VH1-L1-VH2-L2-VL3-L3-VH3-L4-VL2-L5-VL1-CH1-CL; VH1-L1-VH2-L2-VL3-L3-VH3-L4-VL2-L5-VL1-L6-CH1-CL; VH1-L1-VH2-L2-VL3-L3-VH3-L4-VL2-L5-VL1-L6-CH1-L7-CL; VH1-L1-VH2-L2-VL3-L3-VH3-L4-VL2-L5-VL1-CL-CH1; VH1-L1-VH2-L2-VL3-L3-VH3-L4-VL2-L5-VL1-L6-CL-CH1; VH1-L1-VH2-L2-VL3-L3-VH3-L4-VL2-L5-VL1-L6-CL-L7-CH1; VL1-L1-VH2-L2-VH3-L3-VL3-L4-VL2-L5-VH1-CH1; VL1-L1-VH2-L2-VH3-L3-VL3-L4-VL2-L5-VH1-L6-CH1; VL1-L1-VH2-L2-VH3-L3-VL3-L4-VL2-L5-VH1-CL; VL1-L1-VH2-L2-VH3-L3-VL3-L4-VL2-L5-VH1-L6-CL; VL1-L1-VH2-L2-VH3-L3-VL3-L4-VL2-L5-VH1-CH1-CL; VL1-L1-VH2-L2-VH3-L3-VL3-L4-VL2-L5-VH1-L6-CH1-CL; VL1-L1-VH2-L2-VH3-L3-VL3-L4-VL2-L5-VH1-L6-CH1-L7-CL; VL1-L1-VH2-L2-VH3-L3-VL3-L4-VL2-L5-VH1-CL-CH1; VL1-L1-VH2-L2-VH3-L3-VL3-L4-VL2-L5-VH1-L6-CL-CH1; VL1-L1-VH2-L2-VH3-L3-VL3-L4-VL2-L5-VH1-L6-CL-L7-CH1; VH1-L1-VL2-L2-VL3-L3-VH3-L4-VH2-L5-VL1-CH1; VH1-L1-VL2-L2-VL3-L3-VH3-L4-VH2-L5-VL1-L6-CH1; VH1-L1-VL2-L2-VL3-L3-VH3-L4-VH2-L5-VL1-CL; VH1-L1-VL2-L2-VL3-L3-VH3-L4-VH2-L5-VL1-L6-CL; VH1-L1-VL2-L2-VL3-L3-VH3-L4-VH2-L5-VL1-CH1-CL; VH1-L1-VL2-L2-VL3-L3-VH3-L4-VH2-L5-VL1-L6-CH1-CL; VH1-L1-VL2-L2-VL3-L3-VH3-L4-VH2-L5-VL1-L6-CH1-L7-CL; VH1-L1-VL2-L2-VL3-L3-VH3-L4-VH2-L5-VL1-CL-CH1; VH1-L1-VL2-L2-VL3-L3-VH3-L4-VH2-L5-VL1-L6-CL-CH1; or VH1-L1-VL2-L2-VL3-L3-VH3-L4-VH2-L5-VL1-L6-CL-L7-CH1; wherein the second polypeptide has a structure represented by VL4-VH4-CH1; VL4-VH4-CL; VL4-VH4-CH1-CL; VL4-VH4-CL-CH1; VH4-VL4-CH1; VH4-VL4-CL; VH4-VL4-CH1-CL; VH4-VL4-CL-CH1; VL4-L8-VH4-CH1; VL4-L8-VH4-CL; VL4-L8-VH4-CH1-CL; VL4-L8-VH4-CL-CH1; VH4-L8-VL4-CH1; VH4-L8-VL4-CL; VH4-L8-VH4-CH1-CL; VH4-L8-VH4-CL-CH1; VL4-VL5-VH5-VH4-CH1; VL4-VL5-VH5-VH4-CL; VL4-VL5-VH5-VH4-CH1-CL; VL4-VL5-VH5-VH4-CL-CH1; VH4-VH5-VL5-VL4-CH1; VH4-VH5-VL5-VL4-CL; VH4-VH5-VL5-VL4-CH1-CL; VH4-VH5-VL5-VL4-CL-CH1; VL4-L8-VL5-L9-VH5-L10-VH4-CH1; VL4-L8-VL5-L9-VH5-L10-VH4-CL; VL4-L8-VL5-L9-VH5-L10-VH4-CH1-CL; VL4-L8-VL5-L9-VH5-L10-VH4-CL-CH1; VH4-L8-VH5-L9-VL5-L10-VL4-CH1; VH4-L8-VH5-L9-VL5-L10-VL4-CL; VH4-L8-VH5-L9-VL5-L10-VL4-CH1-CL; VH4-L8-VH5-L9-VL5-L10-VL4-CL-CH1; VL4-VL5-VL6-VH6-VH5-VH4-CH1; VL4-VL5-VL6-VH6-VH5-VH4-CL; VL4-VL5-VL6-VH6-VH5-VH4-CH1-CL; VL4-VL5-VL6-VH6-VH5-VH4-CL-CH1; VH4-VH5-VH6-VL6-VL5-VL4-CH1; VH4-VH5-VH6-VL6-VL5-VL4-CL; VH4-VH5-VH6-VL6-VL5-VL4-CH1-CL; VH4-VH5-VH6-VL6-VL5-VL4-CL-CH1; VL4-VH5-VL6-VH6-VL5-VH4-CH1; VL4-VH5-VL6-VH6-VL5-VH4-CL; VL4-VH5-VL6-VH6-VL5-VH4-CH1-CL; VL4-VH5-VL6-VH6-VL5-VH4-CL-CH1; VH4-VL5-VH6-VL6-VH5-VL4-CH1; VH4-VL5-VH6-VL6-VH5-VL4-CL; VH4-VL5-VH6-VL6-VH5-VL4-CH1-CL; VH4-VL5-VH6-VL6-VH5-VL4-CL-CH1; VL4-VL5-VH6-VL6-VH5-VH4-CH1; VL4-VL5-VH6-VL6-VH5-VH4-CL; VL4-VL5-VH6-VL6-VH5-VH4-CH1-CL; VL4-VL5-VH6-VL6-VH5-VH4-CL-CH1; VH4-VH5-VL6-VH6-VL5-VL4-CH1; VH4-VH5-VL6-VH6-VL5-VL4-CL; VH4-VH5-VL6-VH6-VL5-VL4-CH1-CL; VH4-VH5-VL6-VH6-VL5-VL4-CL-CH1; VL4-VH5-VH6-VL6-VL5-VH4-CH1; VL4-VH5-VH6-VL6-VL5-VH4-CL; VL4-VH5-VH6-VL6-VL5-VH4-CH1-CL; VL4-VH5-VH6-VL6-VL5-VH4-CL-CH1; VH4-VL5-VL6-VH6-VH5-VL4-CH1; VH4-VL5-VL6-VH6-VH5-VL4-CL; VH4-VL5-VL6-VH6-VH5-VL4-CH1-CL; VH4-VL5-VL6-VH6-VH5-VL4-CL-CH1; VL4-L8-VL5-L9-VL6-L10-VH6-L11-VH5-L12-VH4-CH1; VL4-L8-VL5-L9-VL6-L10-VH6-L11-VH5-L12-VH4-CL; VL4-L8-VL5-L9-VL6-L10-VH6-L11-VH5-L12-VH4-CH1-CL; VL4-L8-VL5-L9-VL6-L10-VH6-L11-VH5-L12-VH4-CL-CH1; VH4-L8-VH5-L9-VH6-L10-VL6-L11-VL5-L12-VL4-CH1; VH4-L8-VH5-L9-VH6-L10-VL6-L11-VL5-L12-VL4-CL; VH4-L8-VH5-L9-VH6-L10-VL6-L11-VL5-L12-VL4-CH1-CL; VH4-L8-VH5-L9-VH6-L10-VL6-L11-VL5-L12-VL4-CL-CH1; VL4-L8-VH5-L9-VL6-L10-VH6-L11-VL5-L12-VH4-CH1; VL4-L8-VH5-L9-VL6-L10-VH6-L11-VL5-L12-VH4-CL; VL4-L8-VH5-L9-VL6-L10-VH6-L11-VL5-L12-VH4-CH1-CL; VL4-L8-VH5-L9-VL6-L10-VH6-L11-VL5-L12-VH4-CL-CH1; VH4-L8-VL5-L9-VH6-L10-VL6-L11-VH5-L12-VL4-CH1; VH4-L8-VL5-L9-VH6-L10-VL6-L11-VH5-L12-VL4-CL; VH4-L8-VL5-L9-VH6-L10-VL6-L11-VH5-L12-VL4-CH1-CL; VH4-L8-VL5-L9-VH6-L10-VL6-L11-VH5-L12-VL4-CL-CH1; VL4-L8-VL5-L9-VH6-L10-VL6-L11-VH5-L12-VH4-CH1; VL4-L8-VL5-L9-VH6-L10-VL6-L11-VH5-L12-VH4-CL; VL4-L8-VL5-L9-VH6-L10-VL6-L11-VH5-L12-VH4-CH1-CL; VL4-L8-VL5-L9-VH6-L10-VL6-L11-VH5-L12-VH4-CL-CH1; VH4-L8-VH5-L9-VL6-L10-VH6-L11-VL5-L12-VL4-CH1; VH4-L8-VH5-L9-VL6-L10-VH6-L11-VL5-L12-VL4-CL; VH4-L8-VH5-L9-VL6-L10-VH6-L11-VL5-L12-VL4-CH1-CL; VH4-L8-VH5-L9-VL6-L10-VH6-L11-VL5-L12-VL4-CL-CH1; VL4-L8-VH5-L9-VH6-L10-VL6-L11-VL5-L12-VH4-CH1; VL4-L8-VH5-L9-VH6-L10-VL6-L11-VL5-L12-VH4-CL; VL4-L8-VH5-L9-VH6-L10-VL6-L11-VL5-L12-VH4-CH1-CL; VL4-L8-VH5-L9-VH6-L10-VL6-L11-VL5-L12-VH4-CL-CH1; VH4-L8-VL5-L9-VL6-L10-VH6-L11-VH5-L12-VL4-CH1; VH4-L8-VL5-L9-VL6-L10-VH6-L11-VH5-L12-VL4-CL; VH4-L8-VL5-L9-VL6-L10-VH6-L11-VH5-L12-VL4-CH1-CL; VH4-L8-VL5-L9-VL6-L10-VH6-L11-VH5-L12-VL4-CL-CH1; VL4-L8-VL5-L9-VL6-L10-VH6-L11-VH5-L12-VH4-CL-CH1; VL4-L8-VL5-L9-VL6-L10-VH6-L11-VH5-L12-VH4-L13-CL; VL4-L8-VL5-L9-VL6-L10-VH6-L11-VH5-L12-VH4-L13-CH1-CL; VL4-L8-VL5-L9-VL6-L10-VH6-L11-VH5-L12-VH4-L13-CL-CH1; VH4-L8-VH5-L9-VH6-L10-VL6-L11-VL5-L12-VL4-L13-CH1; VH4-L8-VH5-L9-VH6-L10-VL6-L11-VL5-L12-VL4-L13-CL; VH4-L8-VH5-L9-VH6-L10-VL6-L11-VL5-L12-VL4-L13-CH1-CL; VH4-L8-VH5-L9-VH6-L10-VL6-L11-VL5-L12-VL4-L13-CL-CH1; VL4-L8-VH5-L9-VL6-L10-VH6-L11-VL5-L12-VH4-L13-CH1; VL4-L8-VH5-L9-VL6-L10-VH6-L11-VL5-L12-VH4-L13-CL; VL4-L8-VH5-L9-VL6-L10-VH6-L11-VL5-L12-VH4-L13-CH1-CL; VL4-L8-VH5-L9-VL6-L10-VH6-L11-VL5-L12-VH4-L13-CL-CH1; VH4-L8-VL5-L9-VH6-L10-VL6-L11-VH5-L12-VL4-L13-CH1; VH4-L8-VL5-L9-VH6-L10-VL6-L11-VH5-L12-VL4-L13-CL; VH4-L8-VL5-L9-VH6-L10-VL6-L11-VH5-L12-VL4-L13-CH1-CL; VH4-L8-VL5-L9-VH6-L10-VL6-L11-VH5-L12-VL4-L13-CL-CH1; VL4-L8-VL5-L9-VH6-L10-VL6-L11-VH5-L12-VH4-L13-CH1; VL4-L8-VL5-L9-VH6-L10-VL6-L11-VH5-L12-VH4-L13-CL; VL4-L8-VL5-L9-VH6-L10-VL6-L11-VH5-L12-VH4-L13-CH1-CL; VL4-L8-VL5-L9-VH6-L10-VL6-L11-VH5-L12-VH4-L13-CL-CH1; VH4-L8-VH5-L9-VL6-L10-VH6-L11-VL5-L12-VL4-L13-CH1; VH4-L8-VH5-L9-VL6-L10-VH6-L11-VL5-L12-VL4-L13-CL; VH4-L8-VH5-L9-VL6-L10-VH6-L11-VL5-L12-VL4-L13-CH1-CL; VH4-L8-VH5-L9-VL6-L10-VH6-L11-VL5-L12-VL4-L13-CL-CH1; VL4-L8-VH5-L9-VH6-L10-VL6-L11-VL5-L12-VH4-L13-CH1; VL4-L8-VH5-L9-VH6-L10-VL6-L11-VL5-L12-VH4-L13-CL; VL4-L8-VH5-L9-VH6-L10-VL6-L11-VL5-L12-VH4-L13-CH1-CL; VL4-L8-VH5-L9-VH6-L10-VL6-L11-VL5-L12-VH4-L13-CH1; VH4-L8-VL5-L9-VL6-L10-VH6-L11-VH5-L12-VL4-L13-CH1; VH4-L8-VL5-L9-VL6-L10-VH6-L11-VH5-L12-VL4-L13-CL; VH4-L8-VL5-L9-VL6-L10-VH6-L11-VH5-L12-VL4-L13-CH1-CL; or VH4-L8-VL5-L9-VL6-L10-VH6-L11-VH5-L12-VL4-L13-CL-CH1; wherein VL1 is a first immunoglobulin light chain variable region; VL2 is a second immunoglobulin light chain variable region; VL3 is a third immunoglobulin light chain variable region; VL4 is a fourth immunoglobulin light chain variable region; VL5 is a fifth immunoglobulin light chain variable region; VL6 is a sixth immunoglobulin light chain variable region; VH1 is a first immunoglobulin heavy chain variable region; VH2 is a second immunoglobulin heavy chain variable region; VH3 is a third immunoglobulin heavy chain variable region; VH4 is a fourth immunoglobulin heavy chain variable region; VH5 is a fifth immunoglobulin heavy chain variable region; VH6 is a sixth immunoglobulin heavy chain variable region; CH1 is a heavy chain constant region 1; CL is a light chain constant region; and L1, L2, L3, L4, L5, L6, L7, L8, L9, L10, L11, L12 and L13 are amino acid linkers. Any combination of VH1, VH2, VH3 and/or VH4 described above may be used in combination with any combination of VL1, VL2, VL3 and/or VL4 described above. Thus, any first polypeptide described above may be used in any combination with any second polypeptide described above.

In some aspects, an antigen binding polypeptide complex provided herein comprises a first polypeptide and a second polypeptide; wherein the first polypeptide has a structure represented by VL1-VL2-VL3-VH3-VH2-VH1-CH1-Fc; VH1-VH2-VH3-VL3-VL2-VL1-CH1-Fc; VL1-VH2-VL3-VH3-VL2-VH1-CH1-Fc; VH1-VL2-VH3-VL3-VH2-VL1-CH1-Fc; VL1-VL2-VH3-VL3-VH2-VH1-CH1-Fc; VH1-VH2-VL3-VH3-VL2-VL1-CH1-Fc; VL1-VH2-VH3-VL3-VL2-VH1-CH1-Fc; VH1-VL2-VL3-VH3-VH2-VL1-CH1-Fc; VL1-VL2-VL3-VH3-VH2-VH1-CL-Fc; VH1-VH2-VH3-VL3-VL2-VL1-CL-Fc; VL1-VH2-VL3-VH3-VL2-VH1-CL-Fc; VH1-VL2-VH3-VL3-VH2-VL1-CL-Fc; VL1-VL2-VH3-VL3-VH2-VH1-CL-Fc; VH1-VH2-VL3-VH3-VL2-VL1-CL-Fc; VL1-VH2-VH3-VL3-VL2-VH1-CL-Fc; VH1-VL2-VL3-VH3-VH2-VL1-CL-Fc; VL1-VL2-VL3-VH3-VH2-VH1-CH1-CL-Fc; VH1-VH2-VH3-VL3-VL2-VL1-CH1-CL-Fc; VL1-VH2-VL3-VH3-VL2-VH1-CH1-CL-Fc; VH1-VL2-VH3-VL3-VH2-VL1-CH1-CL-Fc; VL1-VL2-VH3-VL3-VH2-VH1-CH1-CL-Fc; VH1-VH2-VL3-VH3-VL2-VL1-CH1-CL-Fc; VL1-VH2-VH3-VL3-VL2-VH1-CH1-CL-Fc; VH1-VL2-VL3-VH3-VH2-VL1-CH1-CL-Fc; VL1-VL2-VL3-VH3-VH2-VH1-CL-CH1-Fc; VH1-VH2-VH3-VL3-VL2-VL1-CL-CH1-Fc; VL1-VH2-VL3-VH3-VL2-VH1-CL-CH1-Fc; VH1-VL2-VH3-VL3-VH2-VL1-CL-CH1-Fc; VL1-VL2-VH3-VL3-VH2-VH1-CL-CH1-Fc; VH1-VH2-VL3-VH3-VL2-VL1-CL-CH1-Fc; VL1-VH2-VH3-VL3-VL2-VH1-CL-CH1-Fc; VH1-VL2-VL3-VH3-VH2-VL1-CL-CH1-Fc; VL1-L1-VL2-L2-VL3-L3-VH3-L4-VH2-L5-VH1-CH1-Fc; VH1-L1-VH2-L2-VH3-L3-VL3-L4-VL2-L5-VL1-CH1-Fc; VL1-L1-VH2-L2-VL3-L3-VH3-L4-VL2-L5-VH1-CH1-Fc; VH1-L1-VL2-L2-VH3-L3-VL3-L4-VH2-L5-VL1-CH1-Fc; VL1-L1-VL2-L2-VH3-L3-VL3-L4-VH2-L5-VH1-CH1-Fc; VH1-L1-VH2-L2-VL3-L3-VH3-L4-VL2-L5-VL1-CH1-Fc; VL1-L1-VH2-L2-VH3-L3-VL3-L4-VL2-L5-VH1-CH1-Fc; VH1-L1-VL2-L2-VL3-L3-VH3-L4-VH2-L5-VL1-CH1-Fc; VL1-L1-VL2-L2-VL3-L3-VH3-L4-VH2-L5-VH1-CL-Fc; VH1-L1-VH2-L2-VH3-L3-VL3-L4-VL2-L5-VL1-CL-Fc; VL1-L1-VH2-L2-VL3-L3-VH3-L4-VL2-L5-VH1-CL-Fc; VH1-L1-VL2-L2-VH3-L3-VL3-L4-VH2-L5-VL1-CL-Fc; VL1-L1-VL2-L2-VH3-L3-VL3-L4-VH2-L5-VH1-CL-Fc; VH1-L1-VH2-L2-VL3-L3-VH3-L4-VL2-L5-VL1-CL-Fc; VL1-L1-VH2-L2-VH3-L3-VL3-L4-VL2-L5-VH1-CL-Fc; VH1-L1-VL2-L2-VL3-L3-VH3-L4-VH2-L5-VL1-CL-Fc; VL1-L1-VL2-L2-VL3-L3-VH3-L4-VH2-L5-VH1-CH1-CL-Fc; VH1-L1-VH2-L2-VH3-L3-VL3-L4-VL2-L5-VL1-CH1-CL-Fc; VL1-L1-VH2-L2-VL3-L3-VH3-L4-VL2-L5-VH1-CH1-CL-Fc; VH1-L1-VL2-L2-VH3-L3-VL3-L4-VH2-L5-VL1-CH1-CL-Fc; VL1-L1-VL2-L2-VH3-L3-VL3-L4-VH2-L5-VH1-CH1-CL-Fc; VH1-L1-VH2-L2-VL3-L3-VH3-L4-VL2-L5-VL1-CH1-CL-Fc; VL1-L1-VH2-L2-VH3-L3-VL3-L4-VL2-L5-VH1-CH1-CL-Fc; VH1-L1-VL2-L2-VL3-L3-VH3-L4-VH2-L5-VL1-CH1-CL-Fc; VL1-L1-VL2-L2-VL3-L3-VH3-L4-VH2-L5-VH1-CL-CH1-Fc; VH1-L1-VH2-L2-VH3-L3-VL3-L4-VL2-L5-VL1-CL-CH1-Fc; VL1-L1-VH2-L2-VL3-L3-VH3-L4-VL2-L5-VH1-CL-CH1-Fc; VH1-L1-VL2-L2-VH3-L3-VL3-L4-VH2-L5-VL1-CL-CH1-Fc; VL1-L1-VL2-L2-VH3-L3-VL3-L4-VH2-L5-VH1-CL-CH1-Fc; VH1-L1-VH2-L2-VL3-L3-VH3-L4-VL2-L5-VL1-CL-CH1-Fc; VL1-L1-VH2-L2-VH3-L3-VL3-L4-VL2-L5-VH1-CL-CH1-Fc; or VH1-L1-VL2-L2-VL3-L3-VH3-L4-VH2-L5-VL1-CL-CH1-Fc; wherein the second polypeptide has a structure represented by Fc; VL4-VH4-CH1-Fc; VL4-VH4-CL-Fc; VL4-VH4-CH1-CL-Fc; VL4-VH4-CL-CH1-Fc; VH4-VL4-CH1-Fc; VH4-VL4-CL-Fc; VH4-VL4-CH1-CL-Fc; VH4-VL4-CL-CH1-Fc; VL4-L6-VH4-CH1-Fc; VL4-L6-VH4-CL-Fc; VL4-L6-VH4-CH1-CL-Fc; VL4-L6-VH4-CL-CH1-Fc; VH4-L6-VL4-CH1-Fc; VH4-L6-VL4-CL-Fc; VH4-L6-VL4-CH1-CL-Fc; VH4-L6-VL4-CL-CH1-Fc; VL4-CL-VH4-CH1-Fc; VH4-CL-VL4-CH1-Fc; VL4-CH1-VH4-CL-Fc; VH4-CH1-VL4-CL-Fc; VL4-L6-CL-L7-VH4-L8-CH1-Fc; VL4-L6-CL-L7-VH4-L8-CH1-L9-Fc; VH4-L6-CL-L7-VL4-L8-CH1-Fc; VH4-L6-CL-L7-VL4-L8-CH1-L9-Fc; VL4-L6-CH1-L7-VH4-L8-CL-Fc; VL4-L6-CH1-L7-VH4-L8-CL-L9-Fc; VH4-L6-CH1-L7-VL4-L8-CL-Fc; VH4-L6-CH1-L7-VL4-L8-CL-L9-Fc; VL4-VL5-VH5-VH4-CH1-Fc; VL4-VL5-VH5-VH4-CL-Fc; VL4-VL5-VH5-VH4-CH1-CL-Fc; VL4-VL5-VH5-VH4-CL-CH1-Fc; VH4-VH5-VL5-VL4-CH1-Fc; VH4-VH5-VL5-VL4-CL-Fc; VH4-VH5-VL5-VL4-CH1-CL-Fc; VH4-VH5-VL5-VL4-CL-CH1-Fc; VL4-L6-VL5-L7-VH5-L8-VH4-CH1-Fc; VL4-L6-VL5-L7-VH5-L8-VH4-CL-Fc; VL4-L6-VL5-L7-VH5-L8-VH4-CH1-CL-Fc; VL4-L6-VL5-L7-VH5-L8-VH4-CL-CH1-Fc; VH4-L6-VH5-L7-VL5-L8-VL4-CH1-Fc; VH4-L6-VH5-L7-VL5-L8-VL4-CL-Fc; VH4-L6-VH5-L7-VL5-L8-VL4-CH1-CL-Fc; VH4-L6-VH5-L7-VL5-L8-VL4-CL-CH1-Fc; VL4-VL5-VL6-VH6-VH5-VH4-CH1-Fc; VL4-VL5-VL6-VH6-VH5-VH4-CL-Fc; VL4-VL5-VL6-VH6-VH5-VH4-CH1-CL-Fe; VL4-VL5-VL6-VH6-VH5-VH4-CL-CH1-Fe; VH4-VH5-VH6-VL6-VL5-VL4-CH1-Fc; VH4-VH5-VH6-VL6-VL5-VL4-CL-Fc; VH4-VH5-VH6-VL6-VL5-VL4-CH1-CL-Fc; VH4-VH5-VH6-VL6-VL5-VL4-CL-CH1-Fc; VL4-VH5-VL6-VH6-VL5-VH4-CH1-Fc; VL4-VH5-VL6-VH6-VL5-VH4-CL-Fc; VL4-VH5-VL6-VH6-VL5-VH4-CH1-CL-Fc; VL4-VH5-VL6-VH6-VL5-VH4-CL-CH1-Fc; VH4-VL5-VH6-VL6-VH5-VL4-CH1-Fc; VH4-VL5-VH6-VL6-VH5-VL4-CL-Fc; VH4-VL5-VH6-VL6-VH5-VL4-CH1-CL-Fc; VH4-VL5-VH6-VL6-VH5-VL4-CL-CH1-Fc; VL4-VL5-VH6-VL6-VH5-VH4-CH1-Fc; VL4-VL5-VH6-VL6-VH5-VH4-CL-Fc; VL4-VL5-VH6-VL6-VH5-VH4-CH1-CL-Fc; VL4-VL5-VH6-VL6-VH5-VH4-CL-CH1-Fc; VH4-VH5-VL6-VH6-VL5-VL4-CH1-Fc; VH4-VH5-VL6-VH6-VL5-VL4-CL-Fc; VH4-VH5-VL6-VH6-VL5-VL4-CH1-CL-Fc; VH4-VH5-VL6-VH6-VL5-VL4-CL-CH1-Fc; VL4-VH5-VH6-VL6-VL5-VH4-CH1-Fc; VL4-VH5-VH6-VL6-VL5-VH4-CL-Fc; VL4-VH5-VH6-VL6-VL5-VH4-CH1-CL-Fc; VL4-VH5-VH6-VL6-VL5-VH4-CL-CH1-Fc; VH4-VL5-VL6-VH6-VH5-VL4-CH1-Fc; VH4-VL5-VL6-VH6-VH5-VL4-CL-Fc; VH4-VL5-VL6-VH6-VH5-VL4-CH1-CL-Fc; VH4-VL5-VL6-VH6-VH5-VL4-CL-CH1-Fc; VL4-L6-VL5-L7-VL6-L8-VH6-L9-VH5-L10-VH4-CH1-Fc; VL4-L6-VL5-L7-VL6-L8-VH6-L9-VH5-L10-VH4-CL-Fc; VL4-L6-VL5-L7-VL6-L8-VH6-L9-VH5-L10-VH4-CH1-CL-Fc; VL4-L6-VL5-L7-VL6-L8-VH6-L9-VH5-L10-VH4-CL-CH1-Fc; VH4-L6-VH5-L7-VH6-L8-VL6-L9-VL5-L10-VL4-CH1-Fc; VH4-L6-VH5-L7-VH6-L8-VL6-L9-VL5-L10-VL4-CL-Fc; VH4-L6-VH5-L7-VH6-L8-VL6-L9-VL5-L10-VL4-CH1-CL-Fc; VH4-L6-VH5-L7-VH6-L8-VL6-L9-VL5-L10-VL4-CL-CH1-Fc; VL4-L6-VH5-L7-VL6-L8-VH6-L9-VL5-L10-VH4-CH1-Fc; VL4-L6-VH5-L7-VL6-L8-VH6-L9-VL5-L10-VH4-CL-Fc; VL4-L6-VH5-L7-VL6-L8-VH6-L9-VL5-L10-VH4-CH1-CL-Fc; VL4-L6-VH5-L7-VL6-L8-VH6-L9-VL5-L10-VH4-CL-CH1-Fc; VH4-L6-VL5-L7-VH6-L8-VL6-L9-VH5-L10-VL4-CH1-Fc; VH4-L6-VL5-L7-VH6-L8-VL6-L9-VH5-L10-VL4-CL-Fc; VH4-L6-VL5-L7-VH6-L8-VL6-L9-VH5-L10-VL4-CH1-CL-Fc; VH4-L6-VL5-L7-VH6-L8-VL6-L9-VH5-L10-VL4-CL-CH1-Fc; VL4-L6-VL5-L7-VH6-L8-VL6-L9-VH5-L10-VH4-CH1-Fc; VL4-L6-VL5-L7-VH6-L8-VL6-L9-VH5-L10-VH4-CL-Fc; VL4-L6-VL5-L7-VH6-L8-VL6-L9-VH5-L10-VH4-CH1-CL-Fc; VL4-L6-VL5-L7-VH6-L8-VL6-L9-VH5-L10-VH4-CL-CH1-Fc; VH4-L6-VH5-L7-VL6-L8-VH6-L9-VL5-L10-VL4-CH1-Fc; VH4-L6-VH5-L7-VL6-L8-VH6-L9-VL5-L10-VL4-CL-Fc; VH4-L6-VH5-L7-VL6-L8-VH6-L9-VL5-L10-VL4-CH1-CL-Fc; VH4-L6-VH5-L7-VL6-L8-VH6-L9-VL5-L10-VL4-CL-CH1-Fc; VL4-L6-VH5-L7-VH6-L8-VL6-L9-VL5-L10-VH4-CH1-Fc; VL4-L6-VH5-L7-VH6-L8-VL6-L9-VL5-L10-VH4-CL-Fe; VL4-L6-VH5-L7-VH6-L8-VL6-L9-VL5-L10-VH4-CH1-CL-Fc; VL4-L6-VH5-L7-VH6-L8-VL6-L9-VL5-L10-VH4-CL-CH1-Fc; VH4-L6-VL5-L7-VL6-L8-VH6-L9-VH5-L10-VL4-CH1-Fc; VH4-L6-VL5-L7-VL6-L8-VH6-L9-VH5-L10-VL4-CL-Fc; VH4-L6-VL5-L7-VL6-L8-VH6-L9-VH5-L10-VL4-CH1-CL-Fc; VH4-L6-VL5-L7-VL6-L8-VH6-L9-VH5-L10-VL4-CL-CH1-Fc; VL4-L6-VL5-L7-VL6-L8-VH6-L9-VH5-L10-VH4-L11-CH1-Fc; VL4-L6-VL5-L7-VL6-L8-VH6-L9-VH5-L10-VH4-L11-CL-Fc; VL4-L6-VL5-L7-VL6-L8-VH6-L9-VH5-L10-VH4-L11-CH1-CL-Fc; VL4-L6-VL5-L7-VL6-L8-VH6-L9-VH5-L10-VH4-L11-CL-CH1-Fc; VH4-L6-VH5-L7-VH6-L8-VL6-L9-VL5-L10-VL4-L11-CH1-Fc; VH4-L6-VH5-L7-VH6-L8-VL6-L9-VL5-L10-VL4-L11-CL-Fc; VH4-L6-VH5-L7-VH6-L8-VL6-L9-VL5-L10-VL4-L11-CH1-CL-Fc; VH4-L6-VH5-L7-VH6-L8-VL6-L9-VL5-L10-VL4-L11-CL-CH1-Fc; VL4-L6-VH5-L7-VL6-L8-VH6-L9-VL5-L10-VH4-L11-CH1-Fc; VL4-L6-VH5-L7-VL6-L8-VH6-L9-VL5-L10-VH4-L11-CL-Fc; VL4-L6-VH5-L7-VL6-L8-VH6-L9-VL5-L10-VH4-L11-CH1-CL-Fc; VL4-L6-VH5-L7-VL6-L8-VH6-L9-VL5-L10-VH4-L11-CL-CH1-Fc; VH4-L6-VL5-L7-VH6-L8-VL6-L9-VH5-L10-VL4-L11-CH1-Fc; VH4-L6-VL5-L7-VH6-L8-VL6-L9-VH5-L10-VL4-L11-CL-Fc; VH4-L6-VL5-L7-VH6-L8-VL6-L9-VH5-L10-VL4-L11-CH1-CL-Fc; VH4-L6-VL5-L7-VH6-L8-VL6-L9-VH5-L10-VL4-L11-CL-CH1-Fc; VL4-L6-VL5-L7-VH6-L8-VL6-L9-VH5-L10-VH4-L11-CH1-Fc; VL4-L6-VL5-L7-VH6-L8-VL6-L9-VH5-L10-VH4-L11-CL-Fc; VL4-L6-VL5-L7-VH6-L8-VL6-L9-VH5-L10-VH4-L11-CH1-CL-Fc; VL4-L6-VL5-L7-VH6-L8-VL6-L9-VH5-L10-VH4-L11-CL-CH1-Fc; VH4-L6-VH5-L7-VL6-L8-VH6-L9-VL5-L10-VL4-L11-CH1-Fc; VH4-L6-VH5-L7-VL6-L8-VH6-L9-VL5-L10-VL4-L11-CL-Fc; VH4-L6-VH5-L7-VL6-L8-VH6-L9-VL5-L10-VL4-L11-CH1-CL-Fc; VH4-L6-VH5-L7-VL6-L8-VH6-L9-VL5-L10-VL4-L11-CL-CH1-Fc; VL4-L6-VH5-L7-VH6-L8-VL6-L9-VL5-L10-VH4-L11-CH1-Fc; VL4-L6-VH5-L7-VH6-L8-VL6-L9-VL5-L10-VH4-L11-CL-Fc; VL4-L6-VH5-L7-VH6-L8-VL6-L9-VL5-L10-VH4-L11-CH1-CL-Fc; VL4-L6-VH5-L7-VH6-L8-VL6-L9-VL5-L10-VH4-L11-CL-CH1-Fc; VH4-L6-VL5-L7-VL6-L8-VH6-L9-VH5-L10-VL4-L11-CH1-Fc; VH4-L6-VL5-L7-VL6-L8-VH6-L9-VH5-L10-VL4-L11-CL-Fc; VH4-L6-VL5-L7-VL6-L8-VH6-L9-VH5-L10-VL4-L11-CH1-CL-Fc; or VH4-L6-VL5-L7-VL6-L8-VH6-L9-VH5-L10-VL4-L11-CL-CH1-Fc; wherein VL1 is a first immunoglobulin light chain variable region; VL2 is a second immunoglobulin light chain variable region; VL3 is a third immunoglobulin light chain variable region; VL4 is a fourth immunoglobulin light chain variable region; VL5 is a fifth immunoglobulin light chain variable region; VL6 is a sixth immunoglobulin light chain variable region; VH1 is a first immunoglobulin heavy chain variable region; VH2 is a second immunoglobulin heavy chain variable region; VH3 is a third immunoglobulin heavy chain variable region; VH4 is a fourth immunoglobulin heavy chain variable region; VH5 is a fifth immunoglobulin heavy chain variable region; VH6 is a sixth immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; CH1 is a heavy chain constant region 1; CL is a light chain constant region; and L1, L2, L3, L4, L5, L6, L7, L8, L9, L10 and L11 are amino acid linkers. Any combination of VH1, VH2, VH3 and/or VH4 described above may be used in combination with any combination of VL1, VL2, VL3 and/or VL4 described above. Thus, any first polypeptide described above may be used in any combination with any second polypeptide described above.

In some aspects, antigen binding polypeptide provided herein has a structure represented by VL1-VL2-VL3-VH3-VH2-VH1-CH3-CH3; VH1-VH2-VH3-VL3-VL2-VL1-CH3-CH3; VL1-VH2-VL3-VH3-VL2-VH1-CH3-CH3; VH1-VL2-VH3-VL3-VH2-VL1-CH3-CH3; VL1-VL2-VH3-VL3-VH2-VH1-CH3-CH3; VH1-VH2-VL3-VH3-VL2-VL1-CH3-CH3; VL1-VH2-VH3-VL3-VL2-VH1-CH3-CH3; VH1-VL2-VL3-VH3-VH2-VL1-CH3-CH3; VL1-L1-VL2-L2-VL3-L3-VH3-L4-VH2-L5-VH1-CH3-CH3; VH1-L1-VH2-L2-VH3-L3-VL3-L4-VL2-L5-VL1-CH3-CH3; VL1-L1-VH2-L2-VL3-L3-VH3-L4-VL2-L5-VH1-CH3-CH3; VH1-L1-VL2-L2-VH3-L3-VL3-L4-VH2-L5-VL1-CH3-CH3; VL1-L1-VL2-L2-VH3-L3-VL3-L4-VH2-L5-VH1-CH3-CH3; VH1-L1-VH2-L2-VL3-L3-VH3-L4-VL2-L5-VL1-CH3-CH3; VL1-L1-VH2-L2-VH3-L3-VL3-L4-VL2-L5-VH1-CH3-CH3; VH1-L1-VL2-L2-VL3-L3-VH3-L4-VH2-L5-VL1-CH3-CH3; VL1-L1-VL2-L2-VL3-L3-VH3-L4-VH2-L5-VH1-L6-CH3-CH3; VH1-L1-VH2-L2-VH3-L3-VL3-L4-VL2-L5-VL1-L6-CH3-CH3; VL1-L1-VH2-L2-VL3-L3-VH3-L4-VL2-L5-VH1-L6-CH3-CH3; VH1-L1-VL2-L2-VH3-L3-VL3-L4-VH2-L5-VL1-L6-CH3-CH3; VL1-L1-VL2-L2-VH3-L3-VL3-L4-VH2-L5-VH1-L6-CH3-CH3; VH1-L1-VH2-L2-VL3-L3-VH3-L4-VL2-L5-VL1-L6-CH3-CH3; VL1-L1-VH2-L2-VH3-L3-VL3-L4-VL2-L5-VH1-L6-CH3-CH3; VH1-L1-VL2-L2-VL3-L3-VH3-L4-VH2-L5-VL1-L6-CH3-CH3; VL1-L1-VL2-L2-VL3-L3-VH3-L4-VH2-L5-VH1-L6-CH3-L7-CH3; VH1-L1-VH2-L2-VH3-L3-VL3-L4-VL2-L5-VL1-L6-CH3-L7-CH3; VL1-L1-VH2-L2-VL3-L3-VH3-L4-VL2-L5-VH1-L6-CH3-L7-CH3; VH1-L1-VL2-L2-VH3-L3-VL3-L4-VH2-L5-VL1-L6-CH3-L7-CH3; VL1-L1-VL2-L2-VH3-L3-VL3-L4-VH2-L5-VH1-L6-CH3-L7-CH3; VH1-L1-VH2-L2-VL3-L3-VH3-L4-VL2-L5-VL1-L6-CH3-L7-CH3; VL1-L1-VH2-L2-VH3-L3-VL3-L4-VL2-L5-VH1-L6-CH3-L7-CH3; or VH1-L1-VL2-L2-VL3-L3-VH3-L4-VH2-L5-VL1-L6-CH3-L7-CH3; wherein VL1 is a first immunoglobulin light chain variable region; VL2 is a second immunoglobulin light chain variable region; VL3 is a third immunoglobulin light chain variable region; VH1 is a first immunoglobulin heavy chain variable region; VH2 is a second immunoglobulin heavy chain variable region; VH3 is a third immunoglobulin heavy chain variable region; CH3 is an immunoglobulin heavy chain constant region 3; and L1, L2, L3, L4, L5, L6 and L7 are amino acid linkers.

In some aspects, an antigen binding polypeptide complex provided herein comprises a first polypeptide, a second polypeptide, and a third polypeptide; wherein the first polypeptide has a structure represented by VL1-VL2-VL3-VH3-VH2-VH1; VH1-VH2-VH3-VL3-VL2-VL1; VL1-VH2-VL3-VH3-VL2-VH1; VH1-VL2-VH3-VL3-VH2-VL1; VL1-VL2-VH3-VL3-VH2-VH1; VH1-VH2-VL3-VH3-VL2-VL1; VL1-VH2-VH3-VL3-VL2-VH1; VH1-VL2-VL3-VH3-VH2-VL1; VL1-L1-VL2-L2-VL3-L3-VH3-L4-VH2-L5-VH1; VH1-L1-VH2-L2-VH3-L3-VL3-L4-VL2-L5-VL1; VL1-L1-VH2-L2-VL3-L3-VH3-L4-VL2-L5-VH1; VH1-L1-VL2-L2-VH3-L3-VL3-L4-VH2-L5-VL1; VL1-L1-VL2-L2-VH3-L3-VL3-L4-VH2-L5-VH1; VH1-L1-VH2-L2-VL3-L3-VH3-L4-VL2-L5-VL1; VL1-L1-VH2-L2-VH3-L3-VL3-L4-VL2-L5-VH1; or VH1-L1-VL2-L2-VL3-L3-VH3-L4-VH2-L5-VL1; wherein the second polypeptide has a structure represented by VL4-VL5; VL4-L6-VL5; VL4-VL5-VL6; or VL4-L6-VL5-L7-VL6; wherein the third polypeptide has a structure represented by VH4-VH5; VH4-L6-VH5; VH4-VH5-VH6; or VH4-L6-VH5-L7-VH6; wherein VL1 is a first immunoglobulin light chain variable region; VL2 is a second immunoglobulin light chain variable region; VL3 is a third immunoglobulin light chain variable region; VL4 is a fourth immunoglobulin light chain variable region; VL5 is a fifth immunoglobulin light chain variable region; VL6 is a sixth immunoglobulin light chain variable region; VH1 is a first immunoglobulin heavy chain variable region; VH2 is a second immunoglobulin heavy chain variable region; VH3 is a third immunoglobulin heavy chain variable region; VH4 is a fourth immunoglobulin heavy chain variable region; VH5 is a fifth immunoglobulin heavy chain variable region; VH6 is a sixth immunoglobulin heavy chain variable region; and L1, L2, L3, L4, L5, L6 and L7 are amino acid linkers. Any combination of VH1, VH2, VH3, VH4, VH5 and/or VH6 described above may be used in combination with any combination of VL1, VL2, VL3, VL4, VL5 and/or VL6 described above. Thus, any first polypeptide described above may be used in any combination with any second polypeptide described above and any third polypeptide described above. The first, second and third polypeptides may be selected independently.

In some aspects, an antigen binding polypeptide complex provided herein comprises a first polypeptide, a second polypeptide, and a third polypeptide; wherein the first polypeptide has a structure represented by VL1-VL2-VL3-VH3-VH2-VH1-Fc; VH1-VH2-VH3-VL3-VL2-VL1-Fc; VL1-VH2-VL3-VH3-VL2-VH1-Fc; VH1-VL2-VH3-VL3-VH2-VL1-Fc; VL1-VL2-VH3-VL3-VH2-VH1-Fc; VH1-VH2-VL3-VH3-VL2-VL1-Fc; VL1-VH2-VH3-VL3-VL2-VH1-Fc; VH1-VL2-VL3-VH3-VH2-VL1-Fc; VL1-L1-VL2-L2-VL3-L3-VH3-L4-VH2-L5-VH1-Fc; VL1-L1-VL2-L2-VL3-L3-VH3-L4-VH2-L5-VH1-L6-Fc; VH1-L1-VH2-L2-VH3-L3-VL3-L4-VL2-L5-VL1-Fc; VH1-L1-VH2-L2-VH3-L3-VL3-L4-VL2-L5-VL1-L6-Fc; VL1-L1-VH2-L2-VL3-L3-VH3-L4-VL2-L5-VH1-Fc; VL1-L1-VH2-L2-VL3-L3-VH3-L4-VL2-L5-VH1-L6-Fc; VH1-L1-VL2-L2-VH3-L3-VL3-L4-VH2-L5-VL1-Fc; VH1-L1-VL2-L2-VH3-L3-VL3-L4-VH2-L5-VL1-L6-Fc; VL1-L1-VL2-L2-VH3-L3-VL3-L4-VH2-L5-VH1-Fc; VL1-L1-VL2-L2-VH3-L3-VL3-L4-VH2-L5-VH1-L6-Fc; VH1-L1-VH2-L2-VL3-L3-VH3-L4-VL2-L5-VL1-Fc; VH1-L1-VH2-L2-VL3-L3-VH3-L4-VL2-L5-VL1-L6-Fc; VL1-L1-VH2-L2-VH3-L3-VL3-L4-VL2-L5-VH1-Fc; VL1-L1-VH2-L2-VH3-L3-VL3-L4-VL2-L5-VH1-L6-Fc; VH1-L1-VL2-L2-VL3-L3-VH3-L4-VH2-L5-VL1-Fc; or VH1-L1-VL2-L2-VL3-L3-VH3-L4-VH2-L5-VL1-L6-Fc; wherein the second polypeptide has a structure represented by VL4-VL5; VL4-L7-VL5; VL4-CL; VL4-L7-CL; VL4-CH1; VL4-L7-CH1; VH4-VH5; VH4-L7-VH5; VH4-CL; VH4-L7-CL; VH4-CH1; VH4-L7-CH1; VL4-VL5-VL6; VL4-L7-VL5-L8-VL6; VL4-VL5-VL6-CL; VL4-L7-VL5-L8-VL6-CL; VL4-L7-VL5-L8-VL6-L9-CL; VL4-VL5-VL6-CH1; VL4-L7-VL5-L8-VL6-CH1; VL4-L7-VL5-L8-VL6-L9-CH1; VH4-VH5-VH6; VH4-L7-VH5-L8-VH6; VH4-VH5-VH6-CL; VH4-L7-VH5-L8-VH6-CL; VH4-L7-VH5-L8-VH6-L9-CL; VH4-VH5-VH6-CH1; VH4-L7-VH5-L8-VH6-CH1; or VH4-L7-VH5-L8-VH6-L9-CH1; wherein the third polypeptide has a structure represented by VH4-VH5-Fc; VH4-L10-VH5-Fc; VH4-L10-VH5-L11-Fc; VH4-CH1-Fc; VH4-L10-CH1-Fc; VH4-L10-CH1-L11-Fc; VH4-CL-Fc; VH4-L10-CL-Fc; VH4-L10-CL-L11-Fc; VH4-VH5-Fc; VH4-L10-VH5-Fc; VH4-L10-VH5-L11-Fc; VH4-VH5-VH6-Fc; VH4-L10-VH5-L11-VH6-Fc; VH4-L10-VH5-L11-VH6-L12-Fc; VH4-VH5-VH6-CH1-Fc; VH4-L10-VH5-L11-VH6-CH1-Fc; VH4-L10-VH5-L11-VH6-L12-CH1-Fc; VH4-L10-VH5-L11-VH6-L12-CH1-L13-Fc; VH4-VH5-VH6-CL-Fc; VH4-L10-VH5-L11-VH6-CL-Fc; VH4-L10-VH5-L11-VH6-L12-CL-Fc; VH4-L10-VH5-L11-VH6-L12-CL-L13-Fc; VL4-VL5-VL6-Fc; VL4-L10-VL5-L11-VL6-Fc; VL4-L10-VL5-L11-VL6-L12-Fc; VL4-VL5-VL6-CH1-Fc; VL4-L10-VL5-L11-VL6-CH1-Fc VL4-L10-VL5-L11-VL6-L12-CH1-Fc; VL4-L10-VL5-L11-VL6-L12-CH1-L13-Fc; VL4-VL5-VL6-CL-Fc; VL4-L10-VL5-L11-VL6-CL-Fc; VL4-L10-VL5-L11-VL6-L12-CL-Fc; or VL4-L10-VL5-L11-VL6-L12-CL-L13-Fc; wherein VL1 is a first immunoglobulin light chain variable region; VL2 is a second immunoglobulin light chain variable region; VL3 is a third immunoglobulin light chain variable region; VL4 is a fourth immunoglobulin light chain variable region; VL5 is a fifth immunoglobulin light chain variable region; VL6 is a sixth immunoglobulin light chain variable region; VH1 is a first immunoglobulin heavy chain variable region; VH2 is a second immunoglobulin heavy chain variable region; VH3 is a third immunoglobulin heavy chain variable region; VH4 is a fourth immunoglobulin heavy chain variable region; VH5 is a fifth immunoglobulin heavy chain variable region; VH6 is a sixth immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; CH1 is a heavy chain constant region 1; CL is a light chain constant region; and L1, L2, L3, L4, L5, L6, L7, L8, L9, L10, L11, L12 and L13 are amino acid linkers. Any combination of VH1, VH2, VH3, VH4, VH5 and/or VH6 described above may be used in combination with any combination of VL1, VL2, VL3, VL4, VL5 and/or VL6 described above. Thus, any first polypeptide described above may be used in any combination with any second polypeptide described above and any third polypeptide described above. The first, second and third polypeptides may be selected independently.

In some aspects of any of the antigen binding polypeptides or antigen binding polypeptide complexes provided herein, the polypeptide comprises one or more CL region. In some aspects, CL comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:35. In some aspects, CL comprises an amino acid sequence having at least 90% to SEQ ID NO: 35. In some aspects, CL comprises the amino acid sequence of SEQ ID NO:35.

In some aspects of any of the antigen binding polypeptides or antigen binding polypeptide complexes provided herein, the polypeptide comprises one or more CH1 region. In some aspects, CH1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:36. In some aspects, CH1 comprises an amino acid sequence having at least 90% to SEQ ID NO: 36. In some aspects, CH1 comprises the amino acid sequence of SEQ ID NO:36.

In some aspects of the antigen binding polypeptides or antigen binding polypeptide complexes provided herein, the polypeptide or complex comprises an Fc region. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:37, 293 and 294. In some aspects, Fc comprises an amino acid sequence having at least 90% to any one of SEQ ID NOs: 37, 293 and 294. In some aspects, Fc comprises the amino acid sequence of any one of SEQ ID NOs:37, 293 and 294. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:37. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:293. In some aspects, Fc comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:294. In some aspects, Fc comprises an amino acid sequence having at least 90% to SEQ ID NO: 37. In some aspects, Fc comprises an amino acid sequence having at least 90% to SEQ ID NO: 293. In some aspects, Fc comprises an amino acid sequence having at least 90% to SEQ ID NO: 294. As used herein, “at least 90% identity” includes at least 91%, 92%, 93%, 94%, 95%, 96%, 97%, 98%, 99% and 100% identity to the recited reference sequence. In some aspects, Fc comprises the amino acid sequence of SEQ ID NO:37. In some aspects, Fc comprises the amino acid sequence of SEQ ID NO:293. In some aspects, Fc comprises the amino acid sequence of SEQ ID NO:294.

In some aspects, an antigen binding polypeptide complex provided herein comprises a first polypeptide comprising a first Fc region and a second polypeptide comprising a second Fc region. In some aspects, the first Fc region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:37, 293 and 294, and the second Fc region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:37, 293 and 294. In some aspects, the first Fc region comprises the amino acid sequence of any one of SEQ ID NOs:37, 293 and 294, and the second Fc region comprises an amino acid sequence of any one of SEQ ID NOs:37, 293 and 294. In some aspects, the first Fc region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:37, and the second Fc region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:37. In some aspects, the first Fc region comprises the amino acid sequence of SEQ ID NO:37, and the second Fc region comprises the amino acid sequence of SEQ ID NO:37. In some aspects, the first Fc region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:293, and the second Fc region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:293. In some aspects, the first Fc region comprises the amino acid sequence of SEQ ID NO:293, and the second Fc region comprises the amino acid sequence of SEQ ID NO:293. In some aspects, the first Fc region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:294, and the second Fc region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:294. In some aspects, the first Fc region comprises the amino acid sequence of SEQ ID NO:294, and the second Fc region comprises the amino acid sequence of SEQ ID NO:294. In some aspects, the first Fc region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:293, and the second Fc region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:294. In some aspects, the first Fc region comprises the amino acid sequence of SEQ ID NO:293, and the second Fc region comprises the amino acid sequence of SEQ ID NO:294. In some aspects, the first Fc region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:294, and the second Fc region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:293. In some aspects, the first Fc region comprises the amino acid sequence of SEQ ID NO:294, and the second Fc region comprises the amino acid sequence of SEQ ID NO:293. In some aspects, (i) the first Fc region comprises an amino acid sequence having at least 90% identity to SEQ ID NO: 37 and (ii) the second Fc region comprises an amino acid sequence having at least 90% identity to SEQ ID NO: 37. In some aspects, (i) the first Fc region comprises an amino acid sequence having at least 90% identity to SEQ ID NO: 37 and (ii) the second Fc region comprises an amino acid sequence having at least 90% identity to SEQ ID NO: 293. In some aspects, (i) the first Fc region comprises an amino acid sequence having at least 90% identity to SEQ ID NO: 37 and (ii) the second Fc region comprises an amino acid sequence having at least 90% identity to SEQ ID NO: 294. In some aspects, (i) the first Fc region comprises an amino acid sequence having at least 90% identity to SEQ ID NO: 293 and (ii) the second Fc region comprises an amino acid sequence having at least 90% identity to SEQ ID NO: 37. In some aspects, (i) the first Fc region comprises an amino acid sequence having at least 90% identity to SEQ ID NO: 293 and (ii) the second Fc region comprises an amino acid sequence having at least 90% identity to SEQ ID NO: 293. In some aspects, (i) the first Fc region comprises an amino acid sequence having at least 90% identity to SEQ ID NO: 293 and (ii) the second Fc region comprises an amino acid sequence having at least 90% identity to SEQ ID NO: 294. In some aspects, (i) the first Fc region comprises an amino acid sequence having at least 90% identity to SEQ ID NO: 294 and (ii) the second Fc region comprises an amino acid sequence having at least 90% identity to SEQ ID NO: 37. In some aspects, (i) the first Fc region comprises an amino acid sequence having at least 90% identity to SEQ ID NO: 294 and (ii) the second Fc region comprises an amino acid sequence having at least 90% identity to SEQ ID NO: 293. In some aspects, (i) the first Fc region comprises an amino acid sequence having at least 90% identity to SEQ ID NO: 294 and (ii) the second Fc region comprises an amino acid sequence having at least 90% identity to SEQ ID NO: 294. As used herein, “at least 90% identity” includes at least 91%, 92%, 93%, 94%, 95%, 96%, 97%, 98%, 99% and 100% identity to the recited reference sequence.

Molecular biology and recombinant DNA methods for making, screening and engineering an antigen binding polypeptide or antigen binding polypeptide complex (e.g., an antibody or antigen binding fragment thereof) containing such sequences are well known and described, for example, in Adair et al. Human Antibodies, 5(1-2):41-47, 1994; Kostelny et al., J Immunol, 148(5):1547-1553, 1992, Shiraiwa et al., Methods, 154:10-20, 2019; and Zola, “Monoclonal Antibodies: A Manual of Techniques,” 1987, 1st Ed., CRC Press; and Steinitz, Human Antibodies, 18(1-2):1-10, 2009.

As used herein, an antigen binding polypeptide or antigen binding polypeptide complex (e.g., an antibody or antigen binding fragment thereof), or region or domain thereof that “specifically binds” refers to its association with an epitope by its antigen binding domain, and that the binding entails some complementarity between the antigen binding domain and the epitope. Specific binding to an epitope occurs where there is binding to that epitope via its antigen binding domain more readily than there would be binding to a random, unrelated epitope.

As used herein, an “epitope” refers to a localized region of an antigen to which an antigen binding polypeptide or antigen binding polypeptide complex (e.g., antibody or antigen binding fragment thereof) can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In some aspects, the epitope to which an antibody or antigen-binding fragment thereof binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). See, e.g., Giege R et al., (1994) Acta Crystallogr D Biol Crystallogr 50(Pt 4): 339-350; McPherson A (1990) Eur J Biochem 189: 1-23; Chayen N E (1997) Structure 5: 1269-1274; McPherson A (1976) J Biol Chem 251: 6300-6303; Meth Enzymol (1985) volumes 114 & 115, eds Wyckoff H W et al., U.S. Pub. No. 2004/0014194), Bricogne G (1993) Acta Crystallogr D Biol Crystallogr 49(Pt 1): 37-60, Bricogne G (1997) Meth Enzymol 276A: 361-423, ed Carter C W, and Roversi et al., (2000) Acta Crystallogr D Biol Crystallogr 56(Pt 10): 1316-1323 (X-ray diffraction crystallography studies); and Champe et al., (1995) J Biol Chem 270: 1388-1394 and Cunningham B C & Wells J A (1989) Science 244: 1081-1085 (mutagenesis mapping).

Specific binding can be represented by a “binding affinity.” Binding affinity refers to an intrinsic binding affinity which reflects a 1:1 interaction between members of a binding pair (e.g., an antigen binding polypeptide complex and an antigen). Binding affinity can be measured and/or expressed in several ways known in the art, including, but not limited to, equilibrium dissociation constant (K_{D}). K_{D} is calculated from the quotient of k_{off}/kₒₙ, where kₒₙ refers to the association rate constant of, e.g., an antigen binding polypeptide complex to an antigen, and k_{off} refers to the dissociation of, e.g., an antigen binding polypeptide complex from an antigen. The kₒₙ and k_{off} can be determined by techniques known to one of ordinary skill in the art, such as Octet® BLI, BIAcore® or KinExA.

Accordingly, in some aspects, an antigen binding polypeptide complex provided herein is an antibody or antigen binding fragment thereof. In some aspects, an antigen binding polypeptide provided herein is part of an antibody or antigen binding fragment thereof.

In some aspects, the antibody or antigen binding fragment thereof specifically binds to an antigen with an equilibrium dissociation constant (K_{D}) of from about 10 μM to about 1 μM. In some aspects, the antibody is IgG, IgM, IgE, IgA or IgD. In some aspects, the IgG is IgG1, IgG2, IgG3 or IgG4. In some aspects, the antigen binding fragment is a Fab, scFab, Fab′, F(ab′)₂, Fv or scFv. In some aspects, the antibody is human or humanized.

In some aspects, an antigen binding polypeptide complex provided herein (e.g., an antibody or antigen binding fragment thereof) is tetravalent. In some aspects, an antigen binding polypeptide complex provided herein (e.g., an antibody or antigen binding fragment thereof) is hexavalent.

Also provided herein is an anti-SARS-CoV2 multivalent antibody that exhibits improved prevention of immune escape compared to (i) a monovalent anti-SARS-CoV2 antibody that binds to one of the same antigens as the anti-SARS-CoV2 multivalent antibody, and/or (ii) a combination of monovalent anti-SARS-CoV2 antibodies that binds to the same antigens as the anti-SARS-CoV2 multivalent antibody.

In some aspects, the anti-SARS-CoV2 multivalent antibody exhibits improved prevention of immune escape compared to a monovalent anti-SARS-CoV2 antibody that binds to one of the same antigens as the anti-SARS-CoV2 multivalent antibody.

In some aspects, the anti-SARS-CoV2 multivalent antibody exhibits increased neutralization potency compared to a combination of monovalent anti-SARS-CoV2 antibodies that binds to the same antigens as the anti-SARS-CoV2 multivalent antibody.

In some aspects, the anti-SARS-CoV2 multispecific antibody prevents immune escape of the anti-SARS-CoV2 multivalent antibody for at least 2, at least 3, at least 4, at least 5, at least 6, or at least 7 selection rounds as measured by an antibody escape assay. In some aspects, the anti-SARS-CoV2 multispecific antibody prevents immune escape of the anti-SARS-CoV2 multivalent antibody for about 2 to about 7 selection rounds as measured by an antibody escape assay, or any value or range of values therein, for example, for about 3 to about 7 selection rounds, about 4 to about 7 selection rounds, about 5 to about 7 selection rounds, about 6 to about 7 selection rounds, about 2 to about 6 selection rounds, about 3 to about 6 selection rounds, about 4 to about 6 selection rounds, about 5 to about 6 selection rounds, about 2 to about 5 selection rounds, about 3 to about 5 selection rounds, about 4 to about 5 selection rounds, about 2 to about 4 selection rounds, about 3 to about 4 selection rounds, or about 2 to about 3 selection rounds as measured by an antibody escape assay. In some aspects, the anti-SARS-CoV2 multispecific antibody prevents immune escape of the anti-SARS-CoV2 multivalent antibody for about 2, about 3, about 4, about 5, about 6 or about 7 selection rounds as measured by an antibody escape assay.

In some aspects, the anti-SARS-CoV2 multispecific antibody prevents immune escape of the anti-SARS-CoV2 multivalent antibody following at least 2, at least 3, at least 4, at least 5, at least 6, or at least 7 exposures to the subject by the SARS-CoV2 virus. In some aspects, the anti-SARS-CoV2 multispecific antibody prevents immune escape of the anti-SARS-CoV2 multivalent antibody following about 2 to about 7 exposures to the subject by the SARS-CoV2 virus, or any value or range of values therein, for example, for about 3 to about 7 exposures, about 4 to about 7 exposures, about 5 to about 7 exposures, about 6 to about 7 exposures, about 2 to about 6 exposures, about 3 to about 6 exposures, about 4 to about 6 exposures, about 5 to about 6 exposures, about 2 to about 5 exposures, about 3 to about 5 exposures, about 4 to about 5 exposures, about 2 to about 4 exposures, about 3 to about 4 exposures, or about 2 to about 3 exposures to the subject by the SARS-CoV2 virus. In some aspects, the anti-SARS-CoV2 multispecific antibody prevents immune escape of the anti-SARS-CoV2 multivalent antibody following about 2, about 3, about 4, about 5, about 6 or about 7 exposures to the subject by the SARS-CoV2 virus.

In some aspects, the anti-SARS-CoV2 multispecific antibody is at least 100-fold, at least 500-fold, at least 1,000-fold, or at least 2,000-fold more effective in inhibiting growth of the SARS-CoV2 virus compared to (i) a monovalent anti-SARS-CoV2 antibody that binds to one of the same antigens as the anti-SARS-CoV2 multivalent antibody and/or (ii) a combination of monovalent anti-SARS-CoV2 antibodies that binds to the same antigens as the anti-SARS-CoV2 multivalent antibody. In some aspects, the anti-SARS-CoV2 multispecific antibody is from about 100-fold to about 2,000-fold more effective in inhibiting growth of the SARS-CoV2 virus compared to (i) a monovalent anti-SARS-CoV2 antibody that binds to one of the same antigens as the anti-SARS-CoV2 multivalent antibody and/or (ii) a combination of monovalent anti-SARS-CoV2 antibodies that binds to the same antigens as the anti-SARS-CoV2 multivalent antibody, or any value or range of values therein, for example, from about 500-fold to about 2,000-fold, about 1,000-fold to about 2,000-fold, about 100-fold to about 1,000-fold, about 500-fold to about 1,000-fold, or about 100-fold to about 500-fold. In some aspects, the anti-SARS-CoV2 multispecific antibody is about 100-fold, about 500-fold, about 1,000-fold, or about 2,000-fold more effective in inhibiting growth of the SARS-CoV2 virus compared to (i) a monovalent anti-SARS-CoV2 antibody that binds to one of the same antigens as the anti-SARS-CoV2 multivalent antibody and/or (ii) a combination of monovalent anti-SARS-CoV2 antibodies that binds to the same antigens as the anti-SARS-CoV2 multivalent antibody.

In some aspects, the anti-SARS-CoV2 multivalent antibody comprises a first antigen binding site that specifically binds to a SARS-CoV-2 protein and a second antigen binding site that specifically binds to a SARS-CoV-2 protein.

In some aspects, the first antigen binding site is different than the second antigen binding site.

In some aspects, the first antigen binding site comprises a first heavy chain variable domain and a first light chain variable domain, and the second antigen binding site comprises a second heavy chain variable domain and a second light chain variable domain.

In some aspects, the first and second heavy chain variable regions each comprise CDR1, CDR2 and CDR3 regions, and the first and second light chain variable region each comprise CDR1, CDR2 and CDR3 regions.

In some aspects, the CDR1 of the first heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75. 171, 179, 187, 264, 272, 280, 288, 513, 521, 529, 537, 545, 553, 561, 569, 577, 585, 593, 601, 609, 617, 639, 647, 656 and 664; the CDR2 of the first heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; the CDR3 of the first heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666; the CDR1 of the first light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:39, 47, 55, 63, 71, 167, 175, 183, 260, 268, 276, 284, 517, 525, 533, 541, 549, 557, 565, 573, 581, 589, 597, 605, 613, 621, 643, 651, 660 and 668; the CDR2 of the first light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:40, 48, 56, 64, 72, 168, 176, 184, 261, 269, 277 (having the sequence of LGS), 285, 518 (having the sequence of DAS), 526 (having the sequence of DVS), 534 (having the sequence of EDS), 542 (having the sequence of KDS), 550 (having the sequence of DAS), 558 (having the sequence of AAS), 566 (having the sequence of GAS), 574 (having the sequence of DDS), 582 (having the sequence of KDS), 590 (having the sequence of SAS), 598 (having the sequence of SAS), 606 (having the sequence of GAS), 614 (having the sequence of GAS), 622 (having the sequence of GAS), 644 (having the sequence of GAS), 652 (having the sequence of SAS), 661 (having the sequence of GAS) and 669 (having the sequence of DAS); the CDR3 of the first light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:41, 49, 57, 65, 73, 169, 177, 185, 262, 270, 278, 286, 519, 527, 535, 543, 551, 559, 567, 575, 583, 591, 599, 607, 615, 623, 645, 653, 662 and 670; the CDR1 of the second heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664; the CDR2 of the second heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; the CDR3 of the second heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571 and 579, 587, 595, 603, 611, 619, 641, 649, 658 and 666; the CDR1 of the second light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:39, 47, 55, 63, 71, 167, 175, 183, 260, 268, 276, 284, 517, 525, 533, 541, 549, 557, 565, 573, 581, 589, 597, 605, 613, 621, 643, 651, 660 and 668; the CDR2 of the second light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:40, 48, 56, 64, 72, 168, 176.184, 261, 269, 277 (having the sequence of LGS), 285, 518 (having the sequence of DAS), 526 (having the sequence of DVS), 534 (having the sequence of EDS), 542 (having the sequence of KDS), 550 (having the sequence of DAS), 558 (having the sequence of AAS), 566 (having the sequence of GAS), 574 (having the sequence of DDS), 582 (having the sequence of KDS), 590 (having the sequence of SAS), 598 (having the sequence of SAS), 606 (having the sequence of GAS), 614 (having the sequence of GAS), 622 (having the sequence of GAS), 644 (having the sequence of GAS), 652 (having the sequence of SAS), 661 (having the sequence of GAS) and 669 (having the sequence of DAS); and the CDR3 of the second light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:41, 49, 57, 65, 73, 169, 177, 185, 262, 270, 278, 286, 519, 527, 535, 543, 551, 559, 567, 575, 583, 591, 599, 607, 615, 623, 645, 653, 662 and 670. In some preferred aspects, CDR1, CDR2 and CDR3 of each of the first heavy chain variable region, the second heavy chain variable region, the first light chain variable region and the second light chain variable region have at least 90% identity with a corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of the first heavy chain variable region, the second heavy chain variable region, the first light chain variable region and the second light chain variable region comprise or consist of a corresponding amino acid sequences specified above.

In some aspects, the first heavy chain variable domain comprises an amino acid sequence having at least 80%, at least 85%, at least 90%, at least 91%, at least 92%, at least 93%, at least 94%, at least 95%, at least 96%, at least 97%, at least 98%, or 100% identity to any one of SEQ ID NOs:42, 50, 58, 66, 74, 170, 178, 186, 263, 271, 279, 287, 510, 512, 520, 528, 536, 544, 552, 560, 568, 576, 584, 592, 600, 608, 616, 624, 626, 628, 630, 632, 634, 636, 638, 646, 654, 663, 779, 781, 785 and 787; the first light chain variable domain comprises an amino acid sequence having at least 80%, at least 85%, at least 90%, at least 91%, at least 92%, at least 93%, at least 94%, at least 95%, at least 96%, at least 97%, at least 98%, or 100% identity to any one of SEQ ID NOs:38, 46, 54, 62, 70, 166, 174, 182, 259, 267, 275, 283, 508, 509, 511, 516, 524, 532, 540, 548, 556, 564, 572, 580, 588, 596, 604, 612, 620, 625, 627, 629, 631, 633, 635, 637, 642, 650, 659 and 667; the second heavy chain variable domain comprises an amino acid sequence having at least 80%, at least 85%, at least 90%, at least 91%, at least 92%, at least 93%, at least 94%, at least 95%, at least 96%, at least 97%, at least 98%, or 100% identity to any one of SEQ ID NOs:42, 50, 58, 66, 74, 170, 178, 186, 263, 271, 279, 287, 510, 512, 520, 528, 536, 544, 552, 560, 568, 576, 584, 592, 600, 608, 616, 624, 626, 628, 630, 632, 634, 636, 638, 646, 654, 663, 779, 781, 785 and 787; and the second light chain variable domain comprises an amino acid sequence having at least 80%, at least 85%, at least 90%, at least 91%, at least 92%, at least 93%, at least 94%, at least 95%, at least 96%, at least 97%, at least 98%, or 100% identity to any one of SEQ ID NOs:38, 46, 54, 62, 70, 166, 174, 182, 259, 267, 275, 283, 508, 509, 511, 516, 524, 532, 540, 548, 556, 564, 572, 580, 588, 596, 604, 612, 620, 625, 627, 629, 631, 633, 635, 637, 642, 650, 659 and 667. In some aspects, the sequence has a mutation at N62, for example, N62Q or N62Q at, for example, SEQ ID NO:279. In some preferred aspects, each of the first heavy chain variable region, the second heavy chain variable region, the first light chain variable region and the second light chain variable region have at least 90% identity with a corresponding amino acid sequences specified above, optionally with a mutation at N62 as described above. As used herein, “at least 90% identity” includes at least 91%, 92%, 93%, 94%, 95%, 96%, 97%, 98%, 99% and 100% identity to the recited reference sequence. In some particularly preferred aspects, each of the first heavy chain variable region, the second heavy chain variable region, the first light chain variable region and the second light chain variable region comprise or consist of a corresponding amino acid sequences specified above, optionally with a mutation at N62 as described above.

In some aspects, the anti-SARS-CoV2 multivalent antibody further comprises a third antigen binding domain.

In some aspects, the third antigen binding site is different than the first antigen binding site, the second antigen binding site, or both the first and second antigen binding sites.

In some aspects, the third antigen binding site comprises a third heavy chain variable domain and a third light chain variable domain.

In some aspects, the third heavy chain variable domain comprises an amino acid sequence having at least 80%, at least 85%, at least 90%, at least 91%, at least 92%, at least 93%, at least 94%, at least 95%, at least 96%, at least 97%, at least 98%, or 100% identity to any one of SEQ ID NOs:42, 50, 58, 66, 74, 170, 178, 186, 263, 271, 279, 287, 510, 512, 520, 528, 536, 544, 552, 560, 568, 576, 584, 592, 600, 608, 616, 624, 626, 628, 630, 632, 634, 636, 638, 646, 654, 663, 779, 781, 785 and 787; and the third light chain variable domain comprises an amino acid sequence having at least 80%, at least 85%, at least 90%, at least 91%, at least 92%, at least 93%, at least 94%, at least 95%, at least 96%, at least 97%, at least 98%, or 100% identity to any one of SEQ ID NOs:38, 46, 54, 62, 70, 166, 174, 182, 259, 267, 275, 283, 508, 509, 511, 516, 524, 532, 540, 548, 556, 564, 572, 580, 588, 596, 604, 612, 620, 625, 627, 629, 631, 633, 635, 637, 642, 650, 659 and 667. In some aspects, the sequence has a mutation at N62, for example, N62Q or N62Q at, for example, SEQ ID NO:279. In some preferred aspects, the third heavy chain variable domain has at least 90% identity with a corresponding amino acid sequences specified above, optionally with a mutation at N62 as described above. As used herein, “at least 90% identity” includes at least 91%, 92%, 93%, 94%, 95%, 96%, 97%, 98%, 99% and 100% identity to the recited reference sequence. In some particularly preferred aspects, the third heavy chain variable domain comprises or consists of a corresponding amino acid sequences specified above, optionally with a mutation at N62 as described above.

In some aspects, the third heavy chain variable region comprises CDR1, CDR2 and CDR3 regions.

In some aspects, the CDR1 of the third heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664; the CDR2 of the third heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; the CDR3 of the third heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666; the CDR1 of the third light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:39, 47, 55, 63, 71, 167, 175, 183, 260, 268, 276, 284, 517, 525, 533, 541, 549, 557, 565, 573, 581, 589, 597, 605, 613, 621, 643, 651, 660 and 668; the CDR2 of the third light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:40, 48, 56, 64, 72, 168, 176, 184, 261, 269, 277 (having the sequence of LGS), 285, 518 (having the sequence of DAS), 526 (having the sequence of DVS), 534 (having the sequence of EDS), 542 (having the sequence of KDS), 550 (having the sequence of DAS), 558 (having the sequence of AAS), 566 (having the sequence of GAS), 574 (having the sequence of DDS), 582 (having the sequence of KDS), 590 (having the sequence of SAS), 598 (having the sequence of SAS), 606 (having the sequence of GAS), 614 (having the sequence of GAS), 622 (having the sequence of GAS), 644 (having the sequence of GAS), 652 (having the sequence of SAS), 661 (having the sequence of GAS) and 669 (having the sequence of DAS); and the CDR3 of the third light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:41, 49, 57, 65, 73, 169, 177, 185, 262, 270, 278, 286, 519, 527, 535, 543, 551, 559, 567, 575, 583, 591, 599, 607, 615, 623, 645, 653, 662 and 670. In some preferred aspects, CDR1, CDR2 and CDR3 of each of the third heavy chain variable region and the third light chain variable region have at least 90% identity with a corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of the third heavy chain variable region and the third light chain variable region comprise or consist of a corresponding amino acid sequences specified above.

In some aspects, the anti-SARS-CoV2 multivalent antibody further comprises a fourth antigen binding domain.

In some aspects, the fourth antigen binding site is different than one or more of the first, second and third antigen binding sites.

In some aspects, the first, second, third and fourth antigen binding sites are different.

In some aspects, the fourth antigen binding site comprises a third heavy chain variable domain and a fourth light chain variable domain.

In some aspects, the fourth heavy chain variable domain comprises an amino acid sequence having at least 80%, at least 85%, at least 90%, at least 91%, at least 92%, at least 93%, at least 94%, at least 95%, at least 96%, at least 97%, at least 98%, or 100% identity to any one of SEQ ID NOs:42, 50, 58, 66, 74, 170, 178, 186, 263, 271, 279, 287, 510, 512, 520, 528, 536, 544, 552, 560, 568, 576, 584, 592, 600, 608, 616, 624, 626, 628, 630, 632, 634, 636, 638, 646, 654, 663, 779, 781, 785 and 787; and the fourth light chain variable domain comprises an amino acid sequence having at least 80%, at least 85%, at least 90%, at least 91%, at least 92%, at least 93%, at least 94%, at least 95%, at least 96%, at least 97%, at least 98%, or 100% identity to any one of SEQ ID NOs:38, 46, 54, 62, 70, 166, 174, 182, 259, 267, 275, 283, 508, 509, 511, 516, 524, 532, 540, 548, 556, 564, 572, 580, 588, 596, 604, 612, 620, 625, 627, 629, 631, 633, 635, 637, 642, 650, 659 and 667. In some aspects, the sequence has a mutation at N62, for example, N62Q or N62Q at, for example, SEQ ID NO:279. In some preferred aspects, the fourth heavy chain variable domain has at least 90% identity with a corresponding amino acid sequences specified above, optionally with a mutation at N62 as described above. As used herein, “at least 90% identity” includes at least 91%, 92%, 93%, 94%, 95%, 96%, 97%, 98%, 99% and 100% identity to the recited reference sequence. In some particularly preferred aspects, the fourth heavy chain variable domain comprises or consists of a corresponding amino acid sequences specified above, optionally with a mutation at N62 as described above.

In some aspects, the fourth heavy chain variable region comprises CDR1, CDR2 and CDR3 regions.

In some aspects, the CDR1 of the fourth heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179,187, 264, 272, 280, 288, 513, 521, 529, 537, 545, 553, 561, 569, 577, 585, 593, 601, 609, 617, 639, 647, 656 and 664; the CDR2 of the fourth heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; the CDR3 of the fourth heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666; the CDR1 of the fourth light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:39, 47, 55, 63, 71, 167, 175, 183, 260, 268, 276, 284, 517, 525, 533, 541, 549, 557, 565, 573, 581, 589, 597, 605, 613, 621, 643, 651, 660 and 668; the CDR2 of the fourth light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:40, 48, 56, 64, 72, 168, 176, 184, 261, 269, 277 (having the sequence of LGS), 285, 518 (having the sequence of DAS), 526 (having the sequence of DVS), 534 (having the sequence of EDS), 542 (having the sequence of KDS), 550 (having the sequence of DAS), 558 (having the sequence of AAS), 566 (having the sequence of GAS), 574 (having the sequence of DDS), 582 (having the sequence of KDS), 590 (having the sequence of SAS), 598 (having the sequence of SAS), 606 (having the sequence of GAS), 614 (having the sequence of GAS), 622 (having the sequence of GAS), 644 (having the sequence of GAS), 652 (having the sequence of SAS), 661 (having the sequence of GAS) and 669 (having the sequence of DAS); and the CDR3 of the fourth light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:41, 49, 57, 65, 73, 169, 177, 185, 262, 270, 278, 286, 519, 527, 535, 543, 551, 559, 567, 575, 583, 591, 599, 607, 615, 623, 645, 653, 662 and 670. In some preferred aspects, CDR1, CDR2 and CDR3 of each of the fourth heavy chain variable region and the fourth light chain variable region have at least 90% identity with a corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of the fourth heavy chain variable region and the fourth light chain variable region comprise or consist of a corresponding amino acid sequences specified above.

In some aspects, VH1, VH2 and/or VH3 (e.g., VH1; V12; VH3; VH1 and V12; VH1 and VH3; VH2 and VH3; or VH1, VH2 and VH3) of an antigen binding polypeptide or antigen binding complex provided herein comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:585; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:586; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:587. In some aspects, CDR1, CDR2 and CDR3 of VH1 have at least 90% identity with the corresponding amino acid sequences specified above. In some aspects, CDR1, CDR2 and CDR3 of VH2 have at least 90% identity with the corresponding amino acid sequences specified above. In some aspects, CDR1, CDR2 and CDR3 of VH3 have at least 90% identity with the corresponding amino acid sequences specified above. In some aspects, CDR1, CDR2 and CDR3 of VH1 and VH2 have at least 90% identity with the corresponding amino acid sequences specified above. In some aspects, CDR1, CDR2 and CDR3 of VH1 and VH3 have at least 90% identity with the corresponding amino acid sequences specified above. In some aspects, CDR1, CDR2 and CDR3 of VH2 and VH3 have at least 90% identity with the corresponding amino acid sequences specified above. In some aspects, CDR1, CDR2 and CDR3 of VH1, VH2 and VH3 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, CDR1, CDR2 and CDR3 of VH1 comprise or consist of the corresponding amino acid sequences specified above. In some aspects, CDR1, CDR2 and CDR3 of VH2 comprise or consist of the corresponding amino acid sequences specified above. In some aspects, CDR1, CDR2 and CDR3 of VH3 comprise or consist of the corresponding amino acid sequences specified above. In some aspects, CDR1, CDR2 and CDR3 of VH1 and VH2 comprise or consist of the corresponding amino acid sequences specified above. In some aspects, CDR1, CDR2 and CDR3 of VH1 and VH3 comprise or consist of the corresponding amino acid sequences specified above. In some aspects, CDR1, CDR2 and CDR3 of VH2 and VH3 comprise or consist of the corresponding amino acid sequences specified above. In some aspects, CDR1, CDR2 and CDR3 of VH1, VH2 and VH3 comprise or consist of the corresponding amino acid sequences specified above.

In some aspects, VL1, VL2 and/or VL3 (e.g., VL1; VL2; VL3; VL1 and VL2; VL1 and VL3; VL2 and VH3; or VL1, VL2 and VL3) comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:589; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:590; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:591. In some aspects, CDR1, CDR2 and CDR3 of VL1 have at least 90% identity with the corresponding amino acid sequences specified above. In some aspects, CDR1, CDR2 and CDR3 of VL2 have at least 90% identity with the corresponding amino acid sequences specified above. In some aspects, CDR1, CDR2 and CDR3 of VL3 have at least 90% identity with the corresponding amino acid sequences specified above. In some aspects, CDR1, CDR2 and CDR3 of VL1 and VL2 have at least 90% identity with the corresponding amino acid sequences specified above. In some aspects, CDR1, CDR2 and CDR3 of VL1 and VL3 have at least 90% identity with the corresponding amino acid sequences specified above. In some aspects, CDR1, CDR2 and CDR3 of VL2 and VL3 have at least 90% identity with the corresponding amino acid sequences specified above. In some aspects, CDR1, CDR2 and CDR3 of VL1, VL2 and VL3 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, CDR1, CDR2 and CDR3 of VL1 comprise or consist of the corresponding amino acid sequences specified above. In some aspects, CDR1, CDR2 and CDR3 of VL2 comprise or consist of the corresponding amino acid sequences specified above. In some aspects, CDR1, CDR2 and CDR3 of VL3 comprise or consist of the corresponding amino acid sequences specified above. In some aspects, CDR1, CDR2 and CDR3 of VL1 and VL2 comprise or consist of the corresponding amino acid sequences specified above. In some aspects, CDR1, CDR2 and CDR3 of VL1 and VL3 comprise or consist of the corresponding amino acid sequences specified above. In some aspects, CDR1, CDR2 and CDR3 of VL2 and VL3 comprise or consist of the corresponding amino acid sequences specified above. In some aspects, CDR1, CDR2 and CDR3 of VL1, VL2 and VL3 comprise or consist of the corresponding amino acid sequences specified above.

In some aspects, VH1, VH2 and/or VH3 (e.g., VH1; V12; VH3; VH1 and V12; VH1 and VH3; VH2 and VH3; or VH1, VH2 and V13) comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:585; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:586; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:587; and VL1, VL2 and/or VL3 (e.g., VL1; VL2; VL3; VL1 and VL2; VL1 and VL3; VL2 and VH3; or VL1, VL2 and VL3) comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:589; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:590; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:591. Any combination of VH1, VH2 and/or VH3 described above may be used in combination with any combination of VL1, VL2 and/or VL3 described above.

In some aspects, VH1, VH2 and/or VH3 (e.g., VH1; V12; VH3; VH1 and V12; VH1 and VH3; VH2 and VH3; or VH1, VH2 and V13) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:584. In some aspects, VH1 has at least 90% identity with the corresponding amino acid sequences specified above. In some aspects, VH2 has at least 90% identity with the corresponding amino acid sequences specified above. In some aspects, VH3 has at least 90% identity with the corresponding amino acid sequences specified above. In some aspects, VH1 and VH2 have at least 90% identity with the corresponding amino acid sequences specified above. In some aspects, VH1 and VH3 have at least 90% identity with the corresponding amino acid sequences specified above. In some aspects, VH2 and VH3 have at least 90% identity with the corresponding amino acid sequences specified above. In some aspects, VH1, VH2 and VH3 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, VH1 comprises or consists of the corresponding amino acid sequences specified above. In some aspects, VH2 comprises or consists of the corresponding amino acid sequences specified above. In some aspects, VH3 comprises or consists of the corresponding amino acid sequences specified above. In some aspects, VH1 and VH2 comprise or consist of the corresponding amino acid sequences specified above. In some aspects, VH1 and VH3 comprise or consist of the corresponding amino acid sequences specified above. In some aspects VH2 and VH3 comprise or consist of the corresponding amino acid sequences specified above. In some aspects VH1, VH2 and VH3 comprise or consist of the corresponding amino acid sequences specified above.

In some aspects, VL1, VL2 and/or VL3 (e.g., VL1; VL2; VL3; VL1 and VL2; VL1 and VL3; VL2 and VL3; or VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:588. In some aspects, VL1 has at least 90% identity with the corresponding amino acid sequences specified above. In some aspects, VL2 has at least 90% identity with the corresponding amino acid sequences specified above. In some aspects, VL3 has at least 90% identity with the corresponding amino acid sequences specified above. In some aspects, VL1 and VL2 have at least 90% identity with the corresponding amino acid sequences specified above. In some aspects, VL1 and VL3 have at least 90% identity with the corresponding amino acid sequences specified above. In some aspects, VL2 and VL3 have at least 90% identity with the corresponding amino acid sequences specified above. In some aspects, VL1, VL2 and VL3 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, VL1 comprises or consists of the corresponding amino acid sequences specified above. In some aspects, VL2 comprises or consists of the corresponding amino acid sequences specified above. In some aspects, VL3 comprises or consists of the corresponding amino acid sequences specified above. In some aspects, VL1 and VL2 comprise or consist of the corresponding amino acid sequences specified above. In some aspects, VL1 and VL3 comprise or consist of the-622-corresponding amino acid sequences specified above. In some aspects VL2 and VL3 comprise or consist of the corresponding amino acid sequences specified above. In some aspects VL1, VL2 and VL3 comprise or consist of the corresponding amino acid sequences specified above.

In some aspects, VH1, VH2 and/or VH3 (e.g., VH1; V12; VH3; VH1 and V12; VH1 and VH3; VH2 and VH3; or VH1, VH2 and V13) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:584; and VL1, VL2 and/or VL3 (e.g., VL1; VL2; VL3; VL1 and VL2; VL1 and VL3; VL2 and VL3; or VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:588. Any combination of VH1, VH2 and/or VH3 described above may be used in combination with any combination of VL1, VL2 and/or VL3 described above.

In some aspects, VH1, VH2 and/or VH3 (e.g., VH1; V12; VH3; VH1 and V12; VH1 and VH3; VH2 and VH3; or VH1, VH2 and V13) comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:593; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:594; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:595. In some aspects, CDR1, CDR2 and CDR3 of VH1 have at least 90% identity with the corresponding amino acid sequences specified above. In some aspects, CDR1, CDR2 and CDR3 of VH2 have at least 90% identity with the corresponding amino acid sequences specified above. In some aspects, CDR1, CDR2 and CDR3 of VH3 have at least 90% identity with the corresponding amino acid sequences specified above. In some aspects, CDR1, CDR2 and CDR3 of VH1 and VH2 have at least 90% identity with the corresponding amino acid sequences specified above. In some aspects, CDR1, CDR2 and CDR3 of VH1 and VH3 have at least 90% identity with the corresponding amino acid sequences specified above. In some aspects, CDR1, CDR2 and CDR3 of VH2 and VH3 have at least 90% identity with the corresponding amino acid sequences specified above. In some aspects, CDR1, CDR2 and CDR3 of VH1, VH2 and VH3 have at least 90% identity with the corresponding amino acid sequences specified above. As used herein, “at least 90% identity” includes at least 91%, 92%, 93%. 94%, 95%, 96%, 97%. 98%, 99% and 100% identity to the recited reference sequence. In some aspects, CDR1, CDR2 and CDR3 of VH1 comprise or consist of the corresponding amino acid sequences specified above. In some aspects, CDR1, CDR2 and CDR3 of VH2 comprise or consist of the corresponding amino acid sequences specified above. In some aspects, CDR1, CDR2 and CDR3 of VH3 comprise or consist of the corresponding amino acid sequences specified above. In some aspects, CDR1, CDR2 and CDR3 of VH1 and VH2 comprise or consist of the corresponding amino acid sequences specified above. In some aspects, CDR1, CDR2 and CDR3 of VH1 and VH3 comprise or consist of the corresponding amino acid sequences specified above. In some aspects, CDR1, CDR2 and CDR3 of VH2 and VH3 comprise or consist of the corresponding amino acid sequences specified above. In some aspects, CDR1, CDR2 and CDR3 of VH1, VH2 and VH3 comprise or consist of the corresponding amino acid sequences specified above.

In some aspects, VL1, VL2 and/or VL3 (e.g., VL1; VL2; VL3; VL1 and VL2; VL1 and VL3; VL2 and VH3; or VL1, VL2 and VL3) comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:597; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:598; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:599. In some aspects, CDR1, CDR2 and CDR3 of VL1 have at least 90% identity with the corresponding amino acid sequences specified above. In some aspects, CDR1, CDR2 and CDR3 of VL2 have at least 90% identity with the corresponding amino acid sequences specified above. In some aspects, CDR1, CDR2 and CDR3 of VL3 have at least 90% identity with the corresponding amino acid sequences specified above. In some aspects, CDR1, CDR2 and CDR3 of VL1 and VL2 have at least 90% identity with the corresponding amino acid sequences specified above. In some aspects, CDR1, CDR2 and CDR3 of VL1 and VL3 have at least 90% identity with the corresponding amino acid sequences specified above. In some aspects, CDR1, CDR2 and CDR3 of VL2 and VL3 have at least 90% identity with the corresponding amino acid sequences specified above. In some aspects, CDR1, CDR2 and CDR3 of VL1, VL2 and VL3 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, CDR1, CDR2 and CDR3 of VL1 comprise or consist of the corresponding amino acid sequences specified above. In some aspects, CDR1, CDR2 and CDR3 of VL2 comprise or consist of the corresponding amino acid sequences specified above. In some aspects, CDR1, CDR2 and CDR3 of VL3 comprise or consist of the corresponding amino acid sequences specified above. In some aspects, CDR1, CDR2 and CDR3 of VL1 and VL2 comprise or consist of the corresponding amino acid sequences specified above. In some aspects, CDR1, CDR2 and CDR3 of VL1 and VL3 comprise or consist of the corresponding amino acid sequences specified above. In some aspects, CDR1, CDR2 and CDR3 of VL2 and VL3 comprise or consist of the corresponding amino acid sequences specified above. In some aspects, CDR1, CDR2 and CDR3 of VL1, VL2 and VL3 comprise or consist of the corresponding amino acid sequences specified above.

In some aspects, VH1, VH2 and/or VH3 (e.g., VH1; V12; VH3; VH1 and V12; VH1 and VH3; VH2 and VH3; or VH1, VH2 and V13) comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:593; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:594; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:595; and VL1, VL2 and/or VL3 (e.g., VL1; VL2; VL3; VL1 and VL2; VL1 and VL3; VL2 and VH3; or VL1, VL2 and VL3) comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:597; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:598; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:599. Any combination of VH1, VH2 and/or VH3 described above may be used in combination with any combination of VL1, VL2 and/or VL3 described above.

In some aspects, VH1, VH2 and/or VH3 (e.g., VH1; V12; VH3; VH1 and V12; VH1 and VH3; VH2 and VH3; or VH1, VH2 and V13) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:592. In some aspects, VH1 has at least 90% identity with the corresponding amino acid sequences specified above. In some aspects, VH2 has at least 90% identity with the corresponding amino acid sequences specified above. In some aspects, VH3 has at least 90% identity with the corresponding amino acid sequences specified above. In some aspects, VH1 and VH2 have at least 90% identity with the corresponding amino acid sequences specified above. In some aspects, VH1 and VH3 have at least 90% identity with the corresponding amino acid sequences specified above. In some aspects, VH2 and VH3 have at least 90% identity with the corresponding amino acid sequences specified above. In some aspects, VH1, VH2 and VH3 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, VH1 comprises or consists of the corresponding amino acid sequences specified above. In some aspects, VH2 comprises or consists of the corresponding amino acid sequences specified above. In some aspects, VH3 comprises or consists of the corresponding amino acid sequences specified above. In some aspects, VH1 and VH2 comprise or consist of the corresponding amino acid sequences specified above. In some aspects, VH1 and VH3 comprise or consist of the corresponding amino acid sequences specified above. In some aspects VH2 and VH3 comprise or consist of the corresponding amino acid sequences specified above. In some aspects VH1, VH2 and VH3 comprise or consist of the corresponding amino acid sequences specified above.

In some aspects, VL1, VL2 and/or VL3 (e.g., VL1; VL2; VL3; VL1 and VL2; VL1 and VL3; VL2 and VL3; or VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:596. In some aspects, VL1 has at least 90% identity with the corresponding amino acid sequences specified above. In some aspects, VL2 has at least 90% identity with the corresponding amino acid sequences specified above. In some aspects, VL3 has at least 90% identity with the corresponding amino acid sequences specified above. In some aspects, VL1 and VL2 have at least 90% identity with the corresponding amino acid sequences specified above. In some aspects, VL1 and VL3 have at least 90% identity with the corresponding amino acid sequences specified above. In some aspects, VL2 and VL3 have at least 90% identity with the corresponding amino acid sequences specified above. In some aspects, VL1, VL2 and VL3 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, VL1 comprises or consists of the corresponding amino acid sequences specified above. In some aspects, VL2 comprises or consists of the corresponding amino acid sequences specified above. In some aspects, VL3 comprises or consists of the corresponding amino acid sequences specified above. In some aspects, VL1 and VL2 comprise or consist of the corresponding amino acid sequences specified above. In some aspects, VL1 and VL3 comprise or consist of the corresponding amino acid sequences specified above. In some aspects VL2 and VL3 comprise or consist of the corresponding amino acid sequences specified above. In some aspects VL1, VL2 and VL3 comprise or consist of the corresponding amino acid sequences specified above.

In some aspects, VH1, VH2 and/or VH3 (e.g., VH1; V12; VH3; VH1 and V12; VH1 and VH3; VH2 and VH3; or VH1, VH2 and V13) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:592; and VL1, VL2 and/or VL3 (e.g., VL1; VL2; VL3; VL1 and VL2; VL1 and VL3; VL2 and VL3; or VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:596. In some aspects, VH1, VH2 and/or VH3 (e.g., VH1; V12; VH3; VH1 and VH2; VH1 and VH3; VH2 and VH3; or VH1, VH2 and V13) comprise an amino acid sequence having at least 90% identity to SEQ ID NO:592; and VL1, VL2 and/or VL3 (e.g., VL1; VL2; VL3; VL1 and VL2; VL1 and VL3; VL2 and VL3; or VL1, VL2 and VL3) comprise an amino acid sequence having at least at least 90% identity to SEQ ID NO:596. Any combination of VH1, VH2 and/or VH3 described above may be used in combination with any combination of VL1, VL2 and/or VL3 described above. As used herein, “at least 90% identity” includes at least 91%, 92%, 93%, 94%, 95%, 96%, 97%, 98%, 99% and 100% identity to the recited reference sequence. In some aspects, VH1, VH2 and/or VH3 (e.g., VH1; V12; VH3; VH1 and V12; VH1 and VH3; VH2 and VH3; or VH1, VH2 and V13) comprise or consist of the amino acid sequence of SEQ ID NO:592; and VL1, VL2 and/or VL3 (e.g., VL1; VL2; VL3; VL1 and VL2; VL1 and VL3; VL2 and VL3; or VL1, VL2 and VL3) comprise or consist of an amino acid sequence of SEQ ID NO:596. Any combination of VH1, VH2 and/or VH3 described above may be used in combination with any combination of VL1, VL2 and/or VL3 described above.

In some aspects, VH1, VH2 and/or VH3 (e.g., VH1; VH2; VH3; VH1 and VH2; VH1 and VH3; VH2 and VH3; or VH1, VH2 and V13) comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:601; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:602; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:603. In some aspects, CDR1, CDR2 and CDR3 of VH1 have at least 90% identity with the corresponding amino acid sequences specified above. In some aspects, CDR1, CDR2 and CDR3 of VH2 have at least 90% identity with the corresponding amino acid sequences specified above. In some aspects, CDR1, CDR2 and CDR3 of VH3 have at least 90% identity with the corresponding amino acid sequences specified above. In some aspects, CDR1, CDR2 and CDR3 of VH1 and VH2 have at least 90% identity with the corresponding amino acid sequences specified above. In some aspects, CDR1, CDR2 and CDR3 of VH1 and VH3 have at least 90% identity with the corresponding amino acid sequences specified above. In some aspects, CDR1, CDR2 and CDR3 of VH2 and VH3 have at least 90% identity with the corresponding amino acid sequences specified above. In some aspects, CDR1, CDR2 and CDR3 of VH1, VH2 and VH3 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, CDR1, CDR2 and CDR3 of VH1 comprise or consist of the corresponding amino acid sequences specified above. In some aspects, CDR1, CDR2 and CDR3 of VH2 comprise or consist of the corresponding amino acid sequences specified above. In some aspects, CDR1, CDR2 and CDR3 of VH3 comprise or consist of the corresponding amino acid sequences specified above. In some aspects, CDR1, CDR2 and CDR3 of VH1 and VH2 comprise or consist of the corresponding amino acid sequences specified above. In some aspects, CDR1, CDR2 and CDR3 of VH1 and VH3 comprise or consist of the corresponding amino acid sequences specified above. In some aspects, CDR1, CDR2 and CDR3 of VH2 and VH3 comprise or consist of the corresponding amino acid sequences specified above. In some aspects, CDR1, CDR2 and CDR3 of VH1, VH2 and VH3 comprise or consist of the corresponding amino acid sequences specified above.

In some aspects, VL1, VL2 and/or VL3 (e.g., VL1; VL2; VL3; VL1 and VL2; VL1 and VL3; VL2 and VH3; or VL1, VL2 and VL3) comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:605; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:606; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:607. In some aspects, CDR1, CDR2 and CDR3 of VL1 have at least 90% identity with the corresponding amino acid sequences specified above. In some aspects, CDR1, CDR2 and CDR3 of VL2 have at least 90% identity with the corresponding amino acid sequences specified above. In some aspects, CDR1, CDR2 and CDR3 of VL3 have at least 90% identity with the corresponding amino acid sequences specified above. In some aspects, CDR1, CDR2 and CDR3 of VL1 and VL2 have at least 90% identity with the corresponding amino acid sequences specified above. In some aspects, CDR1, CDR2 and CDR3 of VL1 and VL3 have at least 90% identity with the corresponding amino acid sequences specified above. In some aspects, CDR1, CDR2 and CDR3 of VL2 and VL3 have at least 90% identity with the corresponding amino acid sequences specified above. In some aspects, CDR1, CDR2 and CDR3 of VL1, VL2 and VL3 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, CDR1, CDR2 and CDR3 of VL1 comprise or consist of the corresponding amino acid sequences specified above. In some aspects, CDR1, CDR2 and CDR3 of VL2 comprise or consist of the corresponding amino acid sequences specified above. In some aspects, CDR1, CDR2 and CDR3 of VL3 comprise or consist of the corresponding amino acid sequences specified above. In some aspects, CDR1, CDR2 and CDR3 of VL1 and VL2 comprise or consist of the corresponding amino acid sequences specified above. In some aspects, CDR1, CDR2 and CDR3 of VL1 and VL3 comprise or consist of the corresponding amino acid sequences specified above. In some aspects, CDR1, CDR2 and CDR3 of VL2 and VL3 comprise or consist of the corresponding amino acid sequences specified above. In some aspects, CDR1, CDR2 and CDR3 of VL1, VL2 and VL3 comprise or consist of the corresponding amino acid sequences specified above.

In some aspects, VH1, VH2 and/or VH3 (e.g., VH1; V12; VH3; VH1 and V12; VH1 and VH3; VH2 and VH3; or VH1, VH2 and V13) comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:601; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:602; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:603; and VL1, VL2 and/or VL3 (e.g., VL1; VL2; VL3; VL1 and VL2; VL1 and VL3; VL2 and VH3; or VL1, VL2 and VL3) comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:605; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:606; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:607. Any combination of VH1, VH2 and/or VH3 described above may be used in combination with any combination of VL1, VL2 and/or VL3 described above.

In some aspects, VH1, VH2 and/or VH3 (e.g., VH1; V12; VH3; VH1 and V12; VH1 and VH3; VH2 and VH3; or VH1, VH2 and V13) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:600. In some aspects, VH1 has at least 90% identity with the corresponding amino acid sequences specified above. In some aspects, VH2 has at least 90% identity with the corresponding amino acid sequences specified above. In some aspects, VH3 has at least 90% identity with the corresponding amino acid sequences specified above. In some aspects, VH1 and VH2 have at least 90% identity with the corresponding amino acid sequences specified above. In some aspects, VH1 and VH3 have at least 90% identity with the corresponding amino acid sequences specified above. In some aspects, VH2 and VH3 have at least 90% identity with the corresponding amino acid sequences specified above. In some aspects, VH1, VH2 and VH3 have at least 90% identity with the corresponding amino acid sequences specified above. In some aspects, VH1 comprises or consists of the corresponding amino acid sequences specified above. In some aspects, VH2 comprises or consists of the corresponding amino acid sequences specified above. In some aspects, VH3 comprises or consists of the corresponding amino acid sequences specified above. In some aspects, VH1 and VH2 comprise or consist of the corresponding amino acid sequences specified above. In some aspects, VH1 and VH3 comprise or consist of the corresponding amino acid sequences specified above. In some aspects VH2 and VH3 comprise or consist of the corresponding amino acid sequences specified above. In some aspects VH1, VH2 and VH3 comprise or consist of the corresponding amino acid sequences specified above.

In some aspects, VL1, VL2 and/or VL3 (e.g., VL1; VL2; VL3; VL1 and VL2; VL1 and VL3; VL2 and VL3; or VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:604. In some aspects, VL1 has at least 90% identity with the corresponding amino acid sequences specified above. In some aspects, VL2 has at least 90% identity with the corresponding amino acid sequences specified above. In some aspects, VL3 has at least 90% identity with the corresponding amino acid sequences specified above. In some aspects, VL1 and VL2 have at least 90% identity with the corresponding amino acid sequences specified above. In some aspects, VL1 and VL3 have at least 90% identity with the corresponding amino acid sequences specified above. In some aspects, VL2 and VL3 have at least 90% identity with the corresponding amino acid sequences specified above. In some aspects, VL1, VL2 and VL3 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, VL1 comprises or consists of the corresponding amino acid sequences specified above. In some aspects, VL2 comprises or consists of the corresponding amino acid sequences specified above. In some aspects, VL3 comprises or consists of the corresponding amino acid sequences specified above. In some aspects, VL1 and VL2 comprise or consist of the corresponding amino acid sequences specified above. In some aspects, VL1 and VL3 comprise or consist of the corresponding amino acid sequences specified above. In some aspects VL2 and VL3 comprise or consist of the corresponding amino acid sequences specified above. In some aspects VL1, VL2 and VL3 comprise or consist of the corresponding amino acid sequences specified above.

In some aspects, VH1, VH2 and/or VH3 (e.g., VH1; V12; VH3; VH1 and V12; VH1 and VH3; VH2 and VH3; or VH1, VH2 and V13) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:600; and VL1, VL2 and/or VL3 (e.g., VL1; VL2; VL3; VL1 and VL2; VL1 and VL3; VL2 and VL3; or VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:604. Any combination of VH1, VH2 and/or VH3 described above may be used in combination with any combination of VL1, VL2 and/or VL3 described above.

In some aspects, VH1, VH2 and/or VH3 (e.g., VH1; V12; VH3; VH1 and V12; VH1 and VH3; VH2 and VH3; or VH1, VH2 and V13) comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:617; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:618; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:619. In some aspects, CDR1, CDR2 and CDR3 of VH1 have at least 90% identity with the corresponding amino acid sequences specified above. In some aspects, CDR1, CDR2 and CDR3 of VH2 have at least 90% identity with the corresponding amino acid sequences specified above. In some aspects, CDR1, CDR2 and CDR3 of VH3 have at least 90% identity with the corresponding amino acid sequences specified above. In some aspects, CDR1, CDR2 and CDR3 of VH1 and VH2 have at least 90% identity with the corresponding amino acid sequences specified above. In some aspects, CDR1, CDR2 and CDR3 of VH1 and VH3 have at least 90% identity with the corresponding amino acid sequences specified above. In some aspects, CDR1, CDR2 and CDR3 of VH2 and VH3 have at least 90% identity with the corresponding amino acid sequences specified above. In some aspects, CDR1, CDR2 and CDR3 of VH1, VH2 and VH3 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, CDR1, CDR2 and CDR3 of VH1 comprise or consist of the corresponding amino acid sequences specified above. In some aspects, CDR1, CDR2 and CDR3 of VH2 comprise or consist of the corresponding amino acid sequences specified above. In some aspects, CDR1, CDR2 and CDR3 of VH3 comprise or consist of the corresponding amino acid sequences specified above. In some aspects, CDR1, CDR2 and CDR3 of VH1 and VH2 comprise or consist of the corresponding amino acid sequences specified above. In some aspects, CDR1, CDR2 and CDR3 of VH1 and VH3 comprise or consist of the corresponding amino acid sequences specified above. In some aspects, CDR1, CDR2 and CDR3 of VH2 and VH3 comprise or consist of the corresponding amino acid sequences specified above. In some aspects, CDR1, CDR2 and CDR3 of VH1, VH2 and VH3 comprise or consist of the corresponding amino acid sequences specified above.

In some aspects, VL1, VL2 and/or VL3 (e.g., VL1; VL2; VL3; VL1 and VL2; VL1 and VL3; VL2 and VH3; or VL1, VL2 and VL3) comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:621; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:622; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:623. In some aspects, CDR1, CDR2 and CDR3 of VL1 have at least 90% identity with the corresponding amino acid sequences specified above. In some aspects, CDR1, CDR2 and CDR3 of VL2 have at least 90% identity with the corresponding amino acid sequences specified above. In some aspects, CDR1, CDR2 and CDR3 of VL3 have at least 90% identity with the corresponding amino acid sequences specified above. In some aspects, CDR1, CDR2 and CDR3 of VL1 and VL2 have at least 90% identity with the corresponding amino acid sequences specified above. In some aspects, CDR1, CDR2 and CDR3 of VL1 and VL3 have at least 90% identity with the corresponding amino acid sequences specified above. In some aspects, CDR1, CDR2 and CDR3 of VL2 and VL3 have at least 90% identity with the corresponding amino acid sequences specified above. In some aspects, CDR1, CDR2 and CDR3 of VL1, VL2 and VL3 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, CDR1, CDR2 and CDR3 of VL1 comprise or consist of the corresponding amino acid sequences specified above. In some aspects, CDR1, CDR2 and CDR3 of VL2 comprise or consist of the corresponding amino acid sequences specified above. In some aspects, CDR1, CDR2 and CDR3 of VL3 comprise or consist of the corresponding amino acid sequences specified above. In some aspects, CDR1, CDR2 and CDR3 of VL1 and VL2 comprise or consist of the corresponding amino acid sequences specified above. In some aspects, CDR1, CDR2 and CDR3 of VL1 and VL3 comprise or consist of the corresponding amino acid sequences specified above. In some aspects, CDR1, CDR2 and CDR3 of VL2 and VL3 comprise or consist of the corresponding amino acid sequences specified above. In some aspects, CDR1, CDR2 and CDR3 of VL1, VL2 and VL3 comprise or consist of the corresponding amino acid sequences specified above.

In some aspects, VH1, VH2 and/or VH3 (e.g., VH1; V12; VH3; VH1 and V12; VH1 and VH3; VH2 and VH3; or VH1, VH2 and V13) comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:617; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:618; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:619; and VL1, VL2 and/or VL3 (e.g., VL1; VL2; VL3; VL1 and VL2; VL1 and VL3; VL2 and VH3; or VL1, VL2 and VL3) comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:621; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:622; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:623. Any combination of VH1, VH2 and/or VH3 described above may be used in combination with any combination of VL1, VL2 and/or VL3 described above.

In some aspects, VH1, VH2 and/or VH3 (e.g., VH1; V12; VH3; VH1 and V12; VH1 and VH3; VH2 and VH3; or VH1, VH2 and V13) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:616. In some aspects, VH1 has at least 90% identity with the corresponding amino acid sequences specified above. In some aspects, VH2 has at least 90% identity with the corresponding amino acid sequences specified above. In some aspects, VH3 has at least 90% identity with the corresponding amino acid sequences specified above. In some aspects, VH1 and VH2 have at least 90% identity with the corresponding amino acid sequences specified above. In some aspects, VH1 and VH3 have at least 90% identity with the corresponding amino acid sequences specified above. In some aspects, VH2 and VH3 have at least 90% identity with the corresponding amino acid sequences specified above. In some aspects, VH1, VH2 and VH3 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, VH1 comprises or consists of the corresponding amino acid sequences specified above. In some aspects, VH2 comprises or consists of the corresponding amino acid sequences specified above. In some aspects, VH3 comprises or consists of the corresponding amino acid sequences specified above. In some aspects, VH1 and VH2 comprise or consist of the corresponding amino acid sequences specified above. In some aspects, VH1 and VH3 comprise or consist of the corresponding amino acid sequences specified above. In some aspects VH2 and VH3 comprise or consist of the corresponding amino acid sequences specified above. In some aspects VH1, VH2 and VH3 comprise or consist of the corresponding amino acid sequences specified above.

In some aspects, VL1, VL2 and/or VL3 (e.g., VL1; VL2; VL3; VL1 and VL2; VL1 and VL3; VL2 and VL3; or VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:620. In some aspects, VL1 has at least 90% identity with the corresponding amino acid sequences specified above. In some aspects, VL2 has at least 90% identity with the corresponding amino acid sequences specified above. In some aspects, VL3 has at least 90% identity with the corresponding amino acid sequences specified above. In some aspects, VL1 and VL2 have at least 90% identity with the corresponding amino acid sequences specified above. In some aspects, VL1 and VL3 have at least 90% identity with the corresponding amino acid sequences specified above. In some aspects, VL2 and VL3 have at least 90% identity with the corresponding amino acid sequences specified above. In some aspects, VL1, VL2 and VL3 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, VL1 comprises or consists of the corresponding amino acid sequences specified above. In some aspects, VL2 comprises or consists of the corresponding amino acid sequences specified above. In some aspects, VL3 comprises or consists of the corresponding amino acid sequences specified above. In some aspects, VL1 and VL2 comprise or consist of the corresponding amino acid sequences specified above. In some aspects, VL1 and VL3 comprise or consist of the corresponding amino acid sequences specified above. In some aspects VL2 and VL3 comprise or consist of the corresponding amino acid sequences specified above. In some aspects VL1, VL2 and VL3 comprise or consist of the corresponding amino acid sequences specified above.

In some aspects, VH1, VH2 and/or VH3 (e.g., VH1; V12; VH3; VH1 and V12; VH1 and VH3; VH2 and VH3; or VH1, VH2 and V13) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:616; and VL1, VL2 and/or VL3 (e.g., VL1; VL2; VL3; VL1 and VL2; VL1 and VL3; VL2 and VL3; or VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:620. Any combination of VH1, VH2 and/or VH3 described above may be used in combination with any combination of VL1, VL2 and/or VL3 described above.

In some aspects, VH1, VH2 and/or VH3 (e.g., VH1; V12; VH3; VH1 and V12; VH1 and VH3; VH2 and VH3; or VH1, VH2 and V13) comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:609; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:610; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:611. In some aspects, CDR1, CDR2 and CDR3 of VH1 have at least 90% identity with the corresponding amino acid sequences specified above. In some aspects, CDR1, CDR2 and CDR3 of VH2 have at least 90% identity with the corresponding amino acid sequences specified above. In some aspects, CDR1, CDR2 and CDR3 of VH3 have at least 90% identity with the corresponding amino acid sequences specified above. In some aspects, CDR1, CDR2 and CDR3 of VH1 and VH2 have at least 90% identity with the corresponding amino acid sequences specified above. In some aspects, CDR1, CDR2 and CDR3 of VH1 and VH3 have at least 90% identity with the corresponding amino acid sequences specified above. In some aspects, CDR1, CDR2 and CDR3 of VH2 and VH3 have at least 90% identity with the corresponding amino acid sequences specified above. In some aspects, CDR1, CDR2 and CDR3 of VH1, VH2 and VH3 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, CDR1, CDR2 and CDR3 of VH1 comprise or consist of the corresponding amino acid sequences specified above. In some aspects, CDR1, CDR2 and CDR3 of VH2 comprise or consist of the corresponding amino acid sequences specified above. In some aspects, CDR1, CDR2 and CDR3 of VH3 comprise or consist of the corresponding amino acid sequences specified above. In some aspects, CDR1, CDR2 and CDR3 of VH1 and VH2 comprise or consist of the corresponding amino acid sequences specified above. In some aspects, CDR1, CDR2 and CDR3 of VH1 and VH3 comprise or consist of the corresponding amino acid sequences specified above. In some aspects, CDR1, CDR2 and CDR3 of VH2 and VH3 comprise or consist of the corresponding amino acid sequences specified above. In some aspects, CDR1, CDR2 and CDR3 of VH1, VH2 and VH3 comprise or consist of the corresponding amino acid sequences specified above.

In some aspects, VL1, VL2 and/or VL3 (e.g., VL1; VL2; VL3; VL1 and VL2; VL1 and VL3; VL2 and VH3; or VL1, VL2 and VL3) comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:613; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:614; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:615. In some aspects, CDR1, CDR2 and CDR3 of VL1 have at least 90% identity with the corresponding amino acid sequences specified above. In some aspects, CDR1, CDR2 and CDR3 of VL2 have at least 90% identity with the corresponding amino acid sequences specified above. In some aspects, CDR1, CDR2 and CDR3 of VL3 have at least 90% identity with the corresponding amino acid sequences specified above. In some aspects, CDR1, CDR2 and CDR3 of VL1 and VL2 have at least 90% identity with the corresponding amino acid sequences specified above. In some aspects, CDR1, CDR2 and CDR3 of VL1 and VL3 have at least 90% identity with the corresponding amino acid sequences specified above. In some aspects, CDR1, CDR2 and CDR3 of VL2 and VL3 have at least 90% identity with the corresponding amino acid sequences specified above. In some aspects, CDR1, CDR2 and CDR3 of VL1, VL2 and VL3 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, CDR1, CDR2 and CDR3 of VL1 comprise or consist of the corresponding amino acid sequences specified above. In some aspects, CDR1, CDR2 and CDR3 of VL2 comprise or consist of the corresponding amino acid sequences specified above. In some aspects, CDR1, CDR2 and CDR3 of VL3 comprise or consist of the corresponding amino acid sequences specified above. In some aspects, CDR1, CDR2 and CDR3 of VL1 and VL2 comprise or consist of the corresponding amino acid sequences specified above. In some aspects, CDR1, CDR2 and CDR3 of VL1 and VL3 comprise or consist of the corresponding amino acid sequences specified above. In some aspects, CDR1, CDR2 and CDR3 of VL2 and VL3 comprise or consist of the corresponding amino acid sequences specified above. In some aspects, CDR1, CDR2 and CDR3 of VL1, VL2 and VL3 comprise or consist of the corresponding amino acid sequences specified above.

In some aspects, VH1, VH2 and/or VH3 (e.g., VH1; V12; VH3; VH1 and V12; VH1 and VH3; VH2 and VH3; or VH1, VH2 and V13) comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:609; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:610; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:611; and VL1, VL2 and/or VL3 (e.g., VL1; VL2; VL3; VL1 and VL2; VL1 and VL3; VL2 and VH3; or VL1, VL2 and VL3) comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:613; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:614; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:615. Any combination of VH1, VH2 and/or VH3 described above may be used in combination with any combination of VL1, VL2 and/or VL3 described above.

In some aspects, VH1, VH2 and/or VH3 (e.g., VH1; V12; VH3; VH1 and V12; VH1 and VH3; VH2 and VH3; or VH1, VH2 and VH3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:608. In some aspects, VH1 has at least 90% identity with the corresponding amino acid sequences specified above. In some aspects, VH2 has at least 90% identity with the corresponding amino acid sequences specified above. In some aspects, VH3 has at least 90% identity with the corresponding amino acid sequences specified above. In some aspects, VH1 and VH2 have at least 90% identity with the corresponding amino acid sequences specified above. In some aspects, VH1 and VH3 have at least 90% identity with the corresponding amino acid sequences specified above. In some aspects, VH2 and VH3 have at least 90% identity with the corresponding amino acid sequences specified above. In some aspects, VH1, VH2 and VH3 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, VH1 comprises or consists of the corresponding amino acid sequences specified above. In some aspects, VH2 comprises or consists of the corresponding amino acid sequences specified above. In some aspects, VH3 comprises or consists of the corresponding amino acid sequences specified above. In some aspects, VH1 and VH2 comprise or consist of the corresponding amino acid sequences specified above. In some aspects, VH1 and VH3 comprise or consist of the corresponding amino acid sequences specified above. In some aspects VH2 and VH3 comprise or consist of the corresponding amino acid sequences specified above. In some aspects VH1, VH2 and VH3 comprise or consist of the corresponding amino acid sequences specified above.

In some aspects, VL1, VL2 and/or VL3 (e.g., VL1; VL2; VL3; VL1 and VL2; VL1 and VL3; VL2 and VL3; or VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:612. In some aspects, VL1 has at least 90% identity with the corresponding amino acid sequences specified above. In some aspects, VL2 has at least 90% identity with the corresponding amino acid sequences specified above. In some aspects, VL3 has at least 90% identity with the corresponding amino acid sequences specified above. In some aspects, VL1 and VL2 have at least 90% identity with the corresponding amino acid sequences specified above. In some aspects, VL1 and VL3 have at least 90% identity with the corresponding amino acid sequences specified above. In some aspects, VL2 and VL3 have at least 90% identity with the corresponding amino acid sequences specified above. In some aspects, VL1, VL2 and VL3 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some aspects, VL1 comprises or consists of the corresponding amino acid sequences specified above. In some aspects, VL2 comprises or consists of the corresponding amino acid sequences specified above. In some aspects, VL3 comprises or consists of the corresponding amino acid sequences specified above. In some aspects, VL1 and VL2 comprise or consist of the corresponding amino acid sequences specified above. In some aspects, VL1 and VL3 comprise or consist of the corresponding amino acid sequences specified above. In some aspects VL2 and VL3 comprise or consist of the corresponding amino acid sequences specified above. In some aspects VL1, VL2 and VL3 comprise or consist of the corresponding amino acid sequences specified above.

In some aspects, VH1, VH2 and/or VH3 (e.g., VH1; V12; VH3; VH1 and V12; VH1 and VH3; VH2 and VH3; or VH1, VH2 and V13) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:608; and VL1, VL2 and/or VL3 (e.g., VL1; VL2; VL3; VL1 and VL2; VL1 and VL3; VL2 and VL3; or VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:612. Any combination of VH1, VH2 and/or VH3 described above may be used in combination with any combination of VL1, VL2 and/or VL3 described above.

In some aspects, the antigen is the SARS-CoV-2 protein. In some aspects, the SARS-CoV-2 protein is a membrane protein, nucleocapsid protein, envelope protein, or spike protein. In some aspects, the SARS-CoV-2 protein is a spike protein.

In some aspects, the SARS-CoV2 virus is a SARS-CoV2 variant.

In some aspects, the SARS-CoV2 variant is Alpha variant (B.1.1.7), Beta variant (B.1.351), Gamma variant (P.1), Delta variant, Epsilon variant (B.1.427), or Omicron variant (B.1.1.529), or a combination thereof.

In some aspects, the Omicron variant is BA. 1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, CH.1.1, or a combination thereof.

In some aspects, any VH and/or VL pair may be deglycosylated of high mannose sugars present on an amino acid or amino acids in the VH and/or VL sequence. Site specific mutation may be performed to replace the glycosylated amino acid with a deglycosylated amino acid substitute.

In some aspects, an antigen binding polypeptide or antigen binding polypeptide complex provided herein has a pI of from about 7 to about 9, from about 7 to about 8, or from about 8 to about 9. In some aspects, an antigen binding polypeptide or antigen binding polypeptide complex provided herein has a pI of about 7, about 7.5, about 8, about 8.5 or about 9. In some aspects, an antigen binding polypeptide or antigen binding polypeptide complex provided herein has a pI of about 6.92, about 6.99, about 6.73, about 8.36, about 7.8, or about 7.92. In some aspects, an antigen binding polypeptide or antigen binding polypeptide complex provided herein has a pI of at least about 7 or at least about 8.

In some aspects, an antigen binding polypeptide or antigen binding polypeptide complex provided herein does not precipitate.

In some aspects, an antigen binding polypeptide or antigen binding polypeptide complex provided herein has a concentration of at least 80 μg/mL in the blood up to about 7 days after infusion (e.g., at 1, 2, 3, 4, 5, 6 or 7 days) of the polypeptide or complex to a subject. In some aspects, an antigen binding polypeptide or antigen binding polypeptide complex provided herein has a concentration of from about 80 μg/mL to about 95 μg/mL in the blood up to about 7 days after infusion (e.g., at 1, 2, 3, 4, 5, 6 or 7 days) of the polypeptide or complex to a subject. In some aspects, an antigen binding polypeptide or antigen binding polypeptide complex provided herein has a concentration of about 80 g/mL, about 85 μg/mL, about 90 μg/mL or about 95 μg/mL in the blood up to about 7 days after infusion (e.g., at 1, 2, 3, 4, 5, 6 or 7 days) of the polypeptide or complex to a subject.

In some aspects, an antigen binding polypeptide or antigen binding polypeptide complex provided herein has a concentration of at least about 5 μg/mL in the blood at from about 14 days to about 21 days (e.g., 14, 15, 16, 18, 18, 19, 20 or 21 days) after infusion of the polypeptide or complex to a subject. In some aspects, an antigen binding polypeptide or antigen binding polypeptide complex provided herein has a concentration of from about 5 μg/mL to about 30 μg/mL in the blood at from about 14 days to about 21 days (e.g., 14, 15, 16, 18, 18, 19, 20 or 21 days) after infusion of the polypeptide or complex to a subject. In some aspects, an antigen binding polypeptide or antigen binding polypeptide complex provided herein has a concentration of from about 5 ug/mL, about g/mL, about 15 μg/mL, about 20 μg/mL, about 25 μg/mL or to about 30 μg/mL in the blood at from about 14 days to about 21 days (e.g., 14, 15, 16, 18, 18, 19, 20 or 21 days) after infusion of the polypeptide or complex to a subject.

In some aspects, an antigen binding polypeptide or antigen binding polypeptide complex provided herein has advantages over a monoclonal antibody or a combination of monoclonal antibodies binding to the same target as the antigen binding polypeptide or antigen binding polypeptide complex. For example, in some aspects, an antigen binding polypeptide or antigen binding polypeptide complex provided herein targets different sites on a SARS-CoV-2 protein. In some aspects, an antigen binding polypeptide or antigen binding polypeptide complex provided herein has synergistic neutralization of a SARS-CoV-2 virus. In some aspects, an antigen binding polypeptide or antigen binding polypeptide complex provided herein has cross coverage of antigenic variants of a SARS-CoV-2 virus. In some aspects, an antigen binding polypeptide or antigen binding polypeptide complex provided herein minimizes SARS-CoV-2 escape. In some aspects, an antigen binding polypeptide or antigen binding polypeptide complex provided herein remains therapeutically effective despite SARS-CoV-2 evolution.

### Amino Acid Linkers

In some aspects, an antigen binding polypeptide or polypeptide complex (e.g., an antibody or antigen binding fragment thereof) provided herein comprises one or more amino acid linkers between two or more regions of the antigen binding polypeptide or polypeptide complex.

As used herein, an “amino acid linker” can be a single amino acid or short amino acid sequence that is capable of joining two regions of an antigen binding polypeptide or polypeptide complex provided herein in a stable manner that maintains or promotes a function associated with the regions. However, as used herein, an “amino acid linker” can also include where an amino acid linker is not present between two regions (referred to herein as an amino acid linker having 0 amino acids). In some aspects, an amino acid linker is represented herein in a structure of an antigen binding polypeptide or polypeptide complex by the abbreviation “l” or “L” and a number (e.g., L1 to denote a first linker, L2 to denote a second linker, L3 to denote a third linker, L4 to denote a fourth linker, L5 to denote a fifth linker, L6 to denote a sixth linker, L7 to denote a seventh linker, L8 to denote an eighth linker, and so on). In some aspects, such enumerated amino acid linkers (e.g., L1) can have the same or different sequence as any other enumerated amino acid linker (e.g., L2, etc.).

In some aspects, an amino acid linker has a length of from 0 amino acids (i.e., an amino acid linker is not present) to about 50 amino acids (e.g., one or more of L1, L2, L3, L4, L5, L6, L7, L8, L9, L10, L11, L12, L13, L14, L15, L16, L17, L18, L19, L20, etc. to L48 or more of a first and/or second polypeptide of an antigen binding polypeptide or polypeptide complex structure provided herein). In some aspects, the amino acid linker has a length of from 0 amino acids to about 45 amino acids, 0 amino acids to about 40 amino acids, 0 amino acids to about 35 amino acids, 0 amino acids to about 30 amino acids, 0 amino acids to about 25 amino acids, 0 amino acids to about 20 amino acids, 0 amino acids to about 15 amino acids, 0 amino acids to about 10 amino acids, 0 amino acids to about 5 amino acids, about 1 amino acid to about 45 amino acids, about 1 amino acid to about 40 amino acids, about 1 amino acid to about 35 amino acids, about 1 amino acid to about 30 amino acids, about 1 amino acid to about 25 amino acids, about 1 amino acid to about 20 amino acids, 1 amino acid to about 15 amino acids, about 1 amino acid to about 10 amino acids, about 1 amino acid to about 5 amino acids, about 5 amino acids to about 50 amino acids, about 5 amino acids to about 45 amino acids, about 5 amino acids to about 40 amino acids, about 5 amino acids to about 35 amino acids, about 5 amino acids to about 30 amino acids, about 5 amino acids to about 25 amino acids, about 5 amino acids to about 20 amino acids, about 5 amino acids to about 15 amino acids, about 5 amino acids to about 10 amino acids, about 10 amino acids to about 50 amino acids, about 10 amino acids to about 45 amino acids, about 10 amino acids to about 40 amino acids, about 10 amino acids to about 35 amino acids, about 10 amino acids to about 30 amino acids, about 10 amino acids to about 25 amino acids, about 10 amino acids to about 20 amino acids, about 10 amino acids to about 15 amino acids, about 15 amino acids to about 50 amino acids, about 15 amino acids to about 45 amino acids, about 15 amino acids to about 40 amino acids, about 15 amino acids to about 35 amino acids, about 15 amino acids to about 30 amino acids, about 15 amino acids to about 25 amino acids, about 15 amino acids to about 20 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 25 amino acids to about 50 amino acids, about 25 amino acids to about 45 amino acids, about 25 amino acids to about 40 amino acids, about 25 amino acids to about 35 amino acids, about 25 amino acids to about 30 amino acids, about 30 amino acids to about 50 amino acids, about 30 amino acids to about 45 amino acids, about 30 amino acids to about 40 amino acids, about 30 amino acids to about 35 amino acids, about 40 amino acids to about 50 amino acids, about 40 amino acids to about 45 amino acids, or about 45 amino acids to about 50 amino acids

In some aspects, the amino acid linker has 0 amino acids (i.e., an amino acid linker is not present) or about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, or about 50 amino acids (e.g., one or more of L1, L2, L3, L4, L5, L6, L7, L8, L9, L10, L11, L12, L13, L14, L15, L16, L17, L18, L19, L20, etc. to L48 or more of a first and/or second polypeptide of an antigen binding polypeptide or polypeptide complex structure provided herein).

In some aspects, the amino acid linker consists of one or more amino acid residues (e.g., one or more of L1, L2, L3, L4, L5, L6, L7, L8, L9, L10, L11, L12, L13, L14, L15, L16, L17, L18, L19, L20, etc. to L48 or more of a first and/or second polypeptide of an antigen binding polypeptide or polypeptide complex structure provided herein). In some aspects, the amino acid residues are selected from the group consisting of glycine, alanine, serine, threonine, cysteine, asparagine, glutamine, leucine, isoleucine, valine, proline, histidine, aspartic acid, glutamic acid, lysine, arginine, methionine, phenylalanine, tryptophan, and tyrosine.

In some aspects, an amino acid linker is non-immunogenic. In some aspects, a non-immunogenic linker consists of serine, glycine and/or alanine residues, or consists of serine and/or glycine residues. In some aspects, an amino acid linker does not contain a T cell epitope or consensus T cell epitope.

In some aspects, an amino acid linker consists of one or more residues of alanine, cysteine, glycine, isoleucine, leucine, methionine, phenylalanine, proline, tryptophan, tyrosine, or valine (e.g., one or more of L1, L2, L3, L4, L5, L6, L7, L8, L9, L10, L11, L12, L13, L14, L15, L16, L17, L18, L19, L20, etc. to L48 or more of a first and/or second polypeptide of an antigen binding polypeptide or polypeptide complex structure provided herein).

Amino acid linker sequences that can be used with the antigen binding polypeptides or polypeptide complexes (e.g., an antibody or antigen binding fragment thereof) provided herein are well known and can be incorporated into the antigen binding polypeptides and polypeptide complexes provided herein using routine molecular biology and recombinant DNA techniques. See, e.g., Chen et al., Adv Drug Deliv Rev., 65(10):1357-1369, 2013; and Chichili et al., Protein Sci., 22(2):153-167, 2013.

In some aspects, an amino acid linker (e.g., one or more of L1, L2, L3, L4, L5, L6, L7, L8, L9, L10, L11, L12, L13, L14, L15, L16, L17, L18, L19, L20, etc. to L48 or more of an antigen binding polypeptide or first and/or second polypeptide of an antigen binding polypeptide complex structure provided herein) comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG.

In some aspects, the amino acid linker between VL1 and VL2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:6.

In some aspects, the amino acid linker between VL2 and VH2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:27.

In some aspects, the amino acid linker between VH2 and VH1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:6.

In some aspects, the amino acid linker between VH1 and Fc comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:12.

In some aspects, the amino acid linker between VL1 and VL2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:6; the amino acid linker between VL2 and VH2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:27; the amino acid linker between VH2 and VH1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:6; and the amino acid linker between VH1 and Fc comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:12.

In some aspects, the amino acid linker between CL and CH1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:27.

In some aspects, the amino acid linker between VL1 and VL2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:6; the amino acid linker between VL2 and VH2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:27; the amino acid linker between VH2 and VH1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:6; and the amino acid linker between CL and CH1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:27.

In some aspects, the amino acid linker between CH1 and Fc comprises 0 amino acids.

In some aspects, the amino acid linker between VL1 and VL2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:6; the amino acid linker between VL2 and VH2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:27; the amino acid linker between VH2 and VH1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:6; and the amino acid linker between CL and CH1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:27; and the amino acid linker between CH1 and Fc comprises 0 amino acids.

In some aspects, the SARS-CoV-2 of the SARS-CoV-2 protein to which an antigen binding polypeptide or polypeptide complex provided herein specifically binds is a variant of concern (VOC). In some aspects, the SARS-CoV-2 of the SARS-CoV-2 protein to which an antigen binding polypeptide or polypeptide complex provided herein specifically binds is a variant under monitoring (e.g., variant with genetic changes suspected to affect virus characteristics and some indication of posing a future risk, but further studies are needed).

In some aspects, the SARS-CoV-2 of the SARS-CoV-2 protein to which an antigen binding polypeptide or polypeptide complex provided herein specifically binds is a Lambda (C.37), Mu (B.1.621), Epsilon (B.1.429, B.1.427, CAL.20C), Zeta (P.2), Eta (B.1.525), Theta (P.3), Iota (B.1.526), or Kappa (B.1.617.1) variant, or a combination thereof. In some aspects, the SARS-CoV-2 of the SARS-CoV-2 protein to which an antigen binding polypeptide or polypeptide complex provided herein specifically binds is a AZ.5, C.1.2, B.1.617.1, B.1.526, B.1.525, B.1.630, B.1.640, AV.1, AT.1, R.1, B.1.466.2, B.1.1.519, C.36.3, B.1.214.2, B.1.427, B.1.429, B.1.1.523, B.1.619, B.1.620, B.1.1.207, B.1.1.317, B.1.616, B.1.618, or B.1.640.2 variant, or a combination thereof.

In some aspects, the SARS-CoV-2 of the SARS-CoV-2 protein to which an antigen binding polypeptide or polypeptide complex provided herein specifically binds is an Alpha (B.1.1.7), Beta (B.1.351), Gamma (P.1), Delta (B.1.617.2), or Omicron (B.1.1.529) variant, or a combination thereof. In some aspects, the Omicron variant is BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, CH.1.1, or a combination thereof.

In some aspects, the SARS-CoV-2 protein has an amino acid mutation of one or more of the following: F2L, L5F, L51, V6F, L7V, P9L, S12C, S13I, Q14H, C15F, N17K, L18F, T20I, T22N, T22A, T22I, Q23K, P25S, A27S, A27V, T29I, F32L, R34C, H49Y, S50L, T51I, Q52L, Q52H, L54F, L54W, F55I, P57L, H69Y, S71F, G72V, T73I, G75V, T76I, F79L, D80N, D80Y, N87Y, D88N, D88E, D88Y, D88A, V90F, T95A, T95I, E96D, K97T, S98F, R1021, 1105L, D111N, K113R, L118F, V130A, E132D, C136R, D138H, L141-, L141F, G142V, G142-, V143F, V143-, Y144-, Y144V, Y145H, H146Y, K147E, N148S, S151I, W152C, M153T, M153V, M153I, E154V, F157L, R158S, L176F, M177I, D178N, G181V, L189F, R190K, I203M, I210-, R214L, D215Y, D215G, L216F, Q218L, F220L, S221L, A222V, A222P, D228H, L229F, Q239R, T240I, L242F, A243S, A243V, H245R, H245Y, R246K, D253Y, D253G, S254F, S256L, W258L, G261V, G261R, A262S, Y265C, V267L, R273S, E281Q, A288S, L293V, D294E, P295S, E298G, T307I, V308L, E309Q, Q314K, Q314L, Q314H, T315I, Q321L, T323I, P330S, A344S, T345S, A348T, A348S, N354K, R357K, V367F, V382L, P384L, V395I, R403K, V407I, A411S, G413R, K417T, N439K, N440K, L441I, G446V, R457K, K458Q, G476S, S477N, P479L, V483A, E484Q, E484K, Q493L, S494P, L452R, Y453F, Y508H, N501Y, H519Q, A520S, A522V, K529E, G545S, T547I, L552F, T553I, E554D, K558N, A570V, T572I, D574Y, E583D, I584V, S5961, 1598V, N603H, Q613H, D614G, V615F, T618A, P621S, V622F, V622I, V622A, A623S, H625Y, A626V, P631S, W633R, G639V, S640F, A647S, A647V, E654Z, E654K, H655Y, N658Y, A668S, A672V, Q675R, Q675H, T676S, T676I, Q677H, Q677R, T678I, P681L, P681R, P681H, R682W, A684S, A684T, V687L, A688V, A688S, S689I, S691F, S698L, N703D, S704L, V705F, A706V, I714M, T716I, I720V, T724A, M7311, T732A, T732I, G744V, D745G, N751D, L754F, R765S, R765H, T768I, G769A, A771S, T778I, Q779H, E780Q, A783S, D808V, D808G, P809S, I818V, L822F, D830H, D830Y, Q836P, Q836L, G838D, A845S, A845D, A845V, A845D, A845S, A846V, R847I, K854R, N856S, T859I, D867N, A879V, A879S, F888L, A893E, A893V, E918V, L922F, A924S, A924V, S929I, D936H, D936Y, L938F, S939F, T941I, G946V, A958S, N969S, L981F, T1006I, V1008T, T1009I, A1016S, A1020V, F1052L, P1053T, L1063F, V1065L, A1070V, Q1071H, E1072V, K1073N, A1078V, A1078S, G1085R, K1086N, R1091L, H1101Y, V1104L, P1112L, D1118Y, Ti 1201, V1122L, S1123P, G1124V, G1124C, V1129A, I1130M, T1136I, D1139H, L1141F, D1146H, S1147L, D1153Y, P1162Q, P1162S, P1162Q, P1162S, D1163Y, G1167V, D1168H, V1176F, N1187Y, K1191N, N1192T, E1195Q, L1203F, K1205N, E1207A, 1219 V, G1219S, I1221T, V1228L, M1229I, V1230L, T1231I, T1231A, C1235F, M1237I, M1237V, M1237T, T1238I, K1245N, C1247F, G1251R, D1259H, S1261F, P1263L, V1264L, a deletion of residues 69 and 70, a deletion of residues 246-252 and D253N, or a combination thereof.

In some aspects, the SARS-CoV-2 protein is a membrane protein, nucleocapsid protein, envelope protein, or spike protein, or a combination thereof. In some aspects, the SARS-CoV-2 protein is a spike protein.

In some aspects, an antigen binding polypeptide complex provided herein has greater neutralization potency against SARS-CoV-2 virus than a monospecific antigen binding polypeptide complex that specifically binds to one of the same antigens. In some aspects, an antigen binding polypeptide complex provided herein is bispecific, trispecific or tetraspecific and has greater neutralization potency against SARS-CoV-2 virus than a monospecific antigen binding polypeptide complex that specifically binds to one of the same antigens the bispecific, trispecific or tetraspecific antigen binding polypeptide complex.

In some aspects, an antigen binding polypeptide complex provided herein has greater neutralization potency against SARS-CoV-2 virus than a mixture of monospecific antigen binding polypeptide complexes that specifically bind to the same antigens. In some aspects, an antigen binding polypeptide complex provided herein is bispecific, trispecific or tetraspecific and has greater neutralization potency against SARS-CoV-2 virus than a mixture of monospecific antigen binding polypeptide complexes that specifically bind to the same antigens as the bispecific, trispecific or tetraspecific antigen binding polypeptide complex.

### Modifications

In some aspects, an antigen binding polypeptide or polypeptide complex (e.g., an antibody or antigen binding fragment thereof) provided herein comprises an effector function mutation or half-life extension mutation.

Effector functions are an important part of the humoral immune response and form an link between innate and adaptive immunity. Most effector functions are induced via the Fc region of an antibody, which can interact with complement proteins and specialized Fc receptors. As used herein, an “effector function mutation” or “Fc effector function mutation” refer to a change in the amino acid sequence, typically in the Fc region, which can increase or decrease effector function, for example, increase binding affinity of Fc for specific Fc receptors, or increase antibody-dependent cellular cytotoxicity (ADCC) activity.

“Half-life” of a pharmaceutically active substance is the time it takes for the amount of the substance, once administered to the body, to reduce by half A “half-life extension mutation” of an antigen binding polypeptide or polypeptide complex provided herein refers to a change in the amino acid sequence, typically in the Fc region, which increases the half-life of the antigen binding polypeptide or polypeptide complex (e.g., by increasing Fc receptor binding affinity, slowing off-rate for Fc and Fc receptors, and/or increased sialylation).

Examples of Fc effector function mutations that decrease or knockout function include, but are not limited to, the following substitutions in the Fc region, based on the EU numbering scheme: L234A, L235A, P239A, N297A, or a combination thereof.

Examples of Fc effector function mutations that increase function include, but are not limited to, the following substitutions in the Fc region, based on the EU numbering scheme: S298A/E333A/K334A, S239D/I332E, S239D/A330L/I332E, G236A/S239D/I332E, or a combination thereof.

Additional examples of effector function mutations, half-life extension mutations and methods for incorporating the same into an amino acid sequence are known and described, for example, in Saunders, “Conceptual Approaches to Modulating Antibody Effector Functions and Circulation Half-Life,” Front. Immunol. Jun. 7, 2019; U.S. Pat. No. 8,546,543, and U.S. Pub. No. 2020/0054765.

In some aspects, an antigen binding polypeptide or polypeptide complex (e.g., an antibody or antigen binding fragment thereof) provided herein comprises one or more knob-into-hole modifications.

The term “knob-into-hole modification,” as used herein, refers to a genetic modification that directs the pairing of two polypeptides to promote heterodimerization. In some aspects, the modification introduces a protuberance (knob) into one polypeptide and a cavity (hole) into the other polypeptide at an interface in which the two polypeptides interact. In some aspects, a knob-into-hole modification can be created by introducing only a hole modification, for example, by replacing an amino acid residue with a smaller side chain than the original amino acid residue (e.g., a substitution of one or more serine, threonine, valine or alanine residues, or a combination thereof). In some aspects, a knob-into-hole modification can be created by introducing only a knob modification, for example, by replacing an amino acid residue with a larger side chain than the original amino acid residue (e.g., a substitution of one or more tryptophan or tyrosine residues, or a combination thereof).

In some aspects, the knob-into-hole modification is in the binding interface of two Fc regions, the binding interface of two CH2 regions, the binding interface of two CH3 regions, the binding interface of a CL region and a CH1 region, or the binding interface of a VH region and a VL region. See, e.g., U.S. Pub. No. 2007/0178552, Int'l Pub. No. WO 96/027011, Int'l Pub. No. WO 98/050431 and Zhu et al., Protein Science 6:781-788, 1987.

In some aspects, the antigen binding polypeptide or polypeptide complex comprises one, two, three, four, five, six, seven, eight, nine, ten, or more knob-into-hole modifications.

Knob-into-hole modifications are well known and can be incorporated into antigen binding polypeptides and polypeptide complexes provided herein using routine molecular biology and recombinant DNA techniques. See, e.g., U.S. Pub. No. 2003/0078385; Int'l Pub. No. WO 96/027011; Ridgway et al., Protein Eng., 9:617-621, 1996; and Merchant et al., Nat. Biotechnol., 16:677-681, 1998.

In some aspects, the knob-into-hole modification is an amino acid substitution. As used herein, such a substitution is described based on the EU numbering scheme of Kabat, which corresponds to the numbering in the Protein Data Bank (PDB).

In some aspects, the knob-into-hole modifications are M428L and N434S or N434A, based on the EU numbering scheme. In some aspects, the knob-into-hole modifications are M428L and N424S, based on the EU numbering scheme. In some aspects, the knob-into-hole modification is N434A, based on the EU numbering scheme.

In some aspects, the knob-into-hole modification is a knob substitution of S354C and/or T366W, based on the EU numbering scheme. In some aspects, the knob-into-hole modification is a knob substitution of S354C, T366W, M428L and N434S, or N434A based on the EU numbering scheme.

In some aspects, the knob-into-hole modification is a hole substitution of Y349C, T366S, L368A, Y407V, M428L, N434S, N434A, M252Y, S254T, T256E, or a combination thereof, based on the EU numbering scheme.

In some aspects, the knob-into-hole modifications are hole substitutions of Y349C, T366S, L368A and Y407V, based on the EU numbering scheme. In some aspects, the knob-into-hole modifications are hole substitutions of M428L and N434S. In some aspects, the knob-into-hole modification is a hole substitution of N434A. In some aspects, the knob-into-hole modifications are hole substitutions of M428L and N434S or N434A, based on the EU numbering scheme. In some aspects, the knob-into-hole modifications are hole substitutions of M252Y, S254T and T256E, based on the EU numbering scheme. In some aspects, the knob-into-hole modifications are hole substitutions of Y349C, T366S, L368A, Y407V, M428L, and N434S.

In some aspects, an antigen binding polypeptide complex is an IgG1 or IgG4 antibody and the knob-into-hole modifications are knob substitutions of S354C and T366W and hole substitutions of Y349C, T366S, L368A and Y407V.

In some aspects, the antigen binding polypeptide complex is an IgG1 or IgG4 antibody and the knob-into-hole modifications are hole substitutions of M428L and N434S or N434A. In some aspects, the knob-into-hole modifications are hole substitutions of M428L and N434S. In some aspects, the knob-into-hole modification is a hole substitution of N434A.

In some aspects, the antigen binding polypeptide complex is an IgG1 or IgG4 antibody and the knob-into-hole modifications are hole substitutions of M252Y, S254T and T256E.

In some aspects, the modifications or mutations can be both in the Fc region and in the antigen binding polypeptide or antigen binding polypeptide complex.

Detectable Labels

In some aspects, an antigen binding polypeptide or polypeptide complex (e.g., an antibody or antigen binding fragment thereof) provided herein comprises one or more detectable labels. An antigen binding polypeptide or polypeptide complex (e.g., an antibody or antigen binding fragment thereof) containing a detectable label is useful in therapeutic, diagnostic, imaging (e.g., radioimaging), or basic research applications.

In some aspects, the detectable label is a radioactive label. Examples of a radioactive label include, but are not limited to, the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²¹I, ¹²⁴I, ¹²⁵I, ¹³¹I, ¹¹¹In, ¹¹⁷Lu, ²¹¹At, ¹⁹⁸Au, ⁶⁷Cu, ²²⁵Ac, ²¹³Bi, ⁹⁹Tc, ¹⁸⁶Re and ⁸⁹Zr.

In some aspects, the detectable label is a chemiluminescent label, fluorescent label, enzyme, biotin, or a combination thereof.

In some aspects, the detectable label is a peptide tag. In some aspects, the peptide tag is located at or near the N-terminus of the polypeptide or polypeptide complex. In some aspects, the peptide tag is located or near at the C-terminus of the polypeptide or polypeptide complex. In some aspects, the peptide tag is an affinity tag or fusion tag.

In some aspects, the detectable label is a polyhistidine tag, polyarginine tag, glutathione-S-transferase (GST), maltose binding protein (MBP), chitin binding protein (CBP), Strep-tag, thioredoxin (TRX), poly(NANP), FLAG tag, ALFA-tag, V5-tag, Myc-tag, hemagglutinin (HA) tag, Spot tag, T7 tag, NE tag, or green fluorescence protein (GFP), or a combination thereof. In some aspects, the polyhistidine tag consists of from about 4 to about 10 histidine residues. In some aspects, the polyhistidine tag consists of about 4, about 5, about 6, about 7, about 8, about 9, or about 10 histidine residues.

Additional examples of detectable labels and methods for introducing detectable labels into a polypeptide are known and include routine chemical, molecular biology and recombinant DNA techniques. See, e.g., Hnatowich et al., Science, 220(4597):613-615, 1983; Yao et al., Int. J. Mol. Sci., 17(2):194, 2016; Kimple et al., Curr. Protoc. Protein Sci., 73:Unit 9.9, 2013; Sambrook J, Fritsch E F. Molecular Cloning: A Laboratory Manual. Cold Spring Harbor Laboratory Press; Cold Spring Harbor, N.Y.: 1989; Molecular Cell Biology, 4^{th} edition, Section 3.5, Purifying, Detecting and Characterizing Proteins; and Mahmoodi et al., Cogent Biology, 5(1):DOI: 10/1080/23312025.2019.1665406.

### Polypeptides, Polynucleotides, Vectors, Cells, and Protein Production Methods

In some aspects, a polypeptide encoding an antigen binding polypeptide or antigen polypeptide complex (e.g., an antibody or antigen binding fragment thereof) is provided herein.

In some aspects, a polypeptide provided herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to one or more of SEQ ID NOs:46-113, 150-165, 190-258, 291, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505 and 711-754. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 46. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 47. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 48. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 49. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 50. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 51. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 52. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 53. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 54. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 55. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 56. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 57. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 58. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 59. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 60. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 61. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 62. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 63. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 64. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 65. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 66. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 67. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 68. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 69. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 70. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 71. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 72. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 73. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 74. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 75. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 76. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 77. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 78. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 79. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 80. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 81. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 82. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 83. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 84. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 85. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 86. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 87. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 88. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 89. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 90. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 91. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 92. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 93. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 94. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 95. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 96. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 97. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 98. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 99. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 100. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 101. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 102. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 103. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 104. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 105. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 106. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 107. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 108. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 109. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 110. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 111. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 112. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 113. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 150. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 151. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 152. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 153. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 154. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 155. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 156. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 157. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 158. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 159. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 160. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 161. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 162. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 163. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 164. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 165. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 190. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 192. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 193. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 194. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 195. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 196. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 197. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 198. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 199. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 200. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 201. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 202. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 203. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 204. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 205. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 206. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 207. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 208. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 209. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 210. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 211. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 212. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 213. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 214. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 215. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 216. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 217. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 218. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 219. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 220. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 221. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 222. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 223. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 224. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 225. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 226. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 227. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 228. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 229. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 230. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 231. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 232. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 233. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 234. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 235. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 236. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 237. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 238. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 239. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 240. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 241. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 242. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 243. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 244. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 245. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 246. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 247. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 248. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 249. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 250. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 251. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 252. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 253. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 254. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 255. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 256. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 257. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 258. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 291. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 295. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 297. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 299. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 301. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 303. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 305. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 307. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 309. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 311. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 313. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 315. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 317. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 319. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 321. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 323. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 325. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 327. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 329. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 331. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 333. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 335. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 337. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 339. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 341. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 343. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 345. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 347. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 3490 In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 351. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 353. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 355. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 357. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 359. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 361. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 363 In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 365. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 367. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 369. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 371. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 373. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 379. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 381. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 383. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 385. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 387. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 389. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 391. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 393. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 395. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 397. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 399. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 401. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 403. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 405. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 407. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 409. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 411. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 413. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 415. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 417. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 419. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 421. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 423. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 425. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 427. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 429. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 431. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 433. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 435. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 437. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 439. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 441. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 443. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 445. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 447. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 449. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 451. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 453. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 455. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 457. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 459. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 461. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 463. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 465. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 467. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 469. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 471. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 473. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 475. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 477. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 479. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 481. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 483. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 485. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 487. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 489. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 491. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 493. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 495. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 497. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 499. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 501. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 503. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 505. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 711. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 712. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 713 In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 714. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 715. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 716. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 717. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 718. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 719. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 720. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 721. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 722. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 723. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 724. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 725. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 726. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 727. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 728. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 729. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 730. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 731. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 732. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 733. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 734. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 735. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 736. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 737. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 738. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 739. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 740. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 741. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 742. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 743. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 744. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 745. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 746. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 747. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 748. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 749. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 750. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 751. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 752. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 753. In some aspects, a polypeptide provided herein comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 754. In some aspects, a polypeptide provided herein comprises the amino acid sequence of one or more of SEQ ID NOs:46-113, 150-165, 190-258, 291, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505 and 711-754.

In some aspects, a polypeptide provided herein comprises an amino acid sequence encoded by one or more polynucleotide having at least 80%, at least 85%, at least 90%, at least 91%, at least 92%, at least 93%, at least 94%, at least 95%, at least 96%, at least 97%, at least 98%, at least 99% or 100% identity to one or more of SEQ ID NOs:114-149, 292, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 393, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 507, 671-710 and 755-777. In some aspects, a polypeptide provided herein comprises or consists of an amino acid sequence encoded by one or more polynucleotide having at least 80% identity to one or more of SEQ ID NOs:114-149, 292, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 393, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 507, 671-710 and 755-777. In some aspects, a polypeptide provided herein comprises or consists of an amino acid sequence encoded by one or more polynucleotide having at least 85% identity to one or more of SEQ ID NOs:114-149, 292, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 393, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 507, 671-710 and 755-777. In some aspects, a polypeptide provided herein comprises or consists of an amino acid sequence encoded by one or more polynucleotide having at least 90% identity to one or more of SEQ ID NOs:114-149, 292, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 393, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 507, 671-710 and 755-777. In some aspects, a polypeptide provided herein comprises or consists of an amino acid sequence encoded by one or more polynucleotide having at least 91% identity to one or more of SEQ ID NOs:114-149, 292, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 393, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 507, 671-710 and 755-777. In some aspects, a polypeptide provided herein comprises or consists of an amino acid sequence encoded by one or more polynucleotide having at least 92% identity to one or more of SEQ ID NOs:114-149, 292, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 393, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 507, 671-710 and 755-777. In some aspects, a polypeptide provided herein comprises or consists of an amino acid sequence encoded by one or more polynucleotide having at least 93% identity to one or more of SEQ ID NOs:114-149, 292, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 393, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 507, 671-710 and 755-777. In some aspects, a polypeptide provided herein comprises or consists of an amino acid sequence encoded by one or more polynucleotide having at least 94% identity to one or more of SEQ ID NOs:114-149, 292, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 393, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 507, 671-710 and 755-777. In some aspects, a polypeptide provided herein comprises or consists of an amino acid sequence encoded by one or more polynucleotide having at least 95% identity to one or more of SEQ ID NOs:114-149, 292, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 393, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 507, 671-710 and 755-777. In some aspects, a polypeptide provided herein comprises or consists of an amino acid sequence encoded by one or more polynucleotide having at least 96% identity to one or more of SEQ ID NOs:114-149, 292, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 393, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 507, 671-710 and 755-777. In some aspects, a polypeptide provided herein comprises or consists of an amino acid sequence encoded by one or more polynucleotide having at least 97% identity to one or more of SEQ ID NOs:114-149, 292, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 393, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 507, 671-710 and 755-777. In some aspects, a polypeptide provided herein comprises or consists of an amino acid sequence encoded by one or more polynucleotide having at least 98% identity to one or more of SEQ ID NOs:114-149, 292, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 393, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 507, 671-710 and 755-777. In some aspects, a polypeptide provided herein comprises or consists of an amino acid sequence encoded by one or more polynucleotide having at least 99% identity to one or more of SEQ ID NOs:114-149, 292, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 393, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 507, 671-710 and 755-777. In some aspects, a polypeptide provided herein comprises or consists of an amino acid sequence encoded by one or more polynucleotide of SEQ ID NOs:114-149, 292, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 393, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 507, 671-710, and 755-777.

In some aspects, a polynucleotide provided herein comprises a sequence having at least 80%, at least 85%, at least 90%, at least 91%, at least 92%, at least 93%, at least 94%, at least 95%, at least 96%, at least 97%, at least 98%, at least 99% or 100% identity to one or more of SEQ ID NOs:114-149, 292, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 393, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 507, 671-710 and 755-777. In some aspects, a polynucleotide provided herein comprises or consists of a polynucleotide sequence having at least 80% identity to one or more of SEQ ID NOs:114-149, 292, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 393, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 507, 671-710 and 755-777. In some aspects, a polynucleotide provided herein comprises or consists of a polynucleotide sequence having at least 85% identity to one or more of SEQ ID NOs:114-149, 292, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 393, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 507, 671-710 and 755-777. In some aspects, a polynucleotide provided herein comprises or consists of a polynucleotide sequence having at least 90% identity to one or more of SEQ ID NOs:114-149, 292, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 393, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 507, 671-710 and 755-777. In some aspects, a polynucleotide provided herein comprises or consists of a polynucleotide sequence having at least 91% identity to one or more of SEQ ID NOs:114-149, 292, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 393, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 507, 671-710 and 755-777. In some aspects, a polynucleotide provided herein comprises or consists of a polynucleotide sequence having at least 92% identity to one or more of SEQ ID NOs:114-149, 292, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 393, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 507, 671-710 and 755-777. In some aspects, a polynucleotide provided herein comprises or consists of a polynucleotide sequence having at least 93% identity to one or more of SEQ ID NOs:114-149, 292, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 393, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 507, 671-710 and 755-777. In some aspects, a polynucleotide provided herein comprises or consists of a polynucleotide sequence having at least 94% identity to one or more of SEQ ID NOs:114-149, 292, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 393, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 507, 671-710 and 755-777. In some aspects, a polynucleotide provided herein comprises or consists of a polynucleotide sequence having at least 95% identity to one or more of SEQ ID NOs:114-149, 292, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 393, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 507, 671-710 and 755-777. In some aspects, a polynucleotide provided herein comprises or consists of a polynucleotide sequence having at least 96% identity to one or more of SEQ ID NOs:114-149, 292, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 393, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 507, 671-710 and 755-777. In some aspects, a polynucleotide provided herein comprises or consists of a polynucleotide sequence having at least 97% identity to one or more of SEQ ID NOs:114-149, 292, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 393, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 507, 671-710 and 755-777. In some aspects, a polynucleotide provided herein comprises or consists of a polynucleotide sequence having at least 98% identity to one or more of SEQ ID NOs:114-149, 292, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 393, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 507, 671-710 and 755-777. In some aspects, a polynucleotide provided herein comprises or consists of a polynucleotide sequence having at least 99% identity to one or more of SEQ ID NOs:114-149, 292, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 393, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 507, 671-710 and 755-777. In some aspects, a polynucleotide provided herein comprises or consists of the sequence of one or more of SEQ ID NOs:114-149, 292, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 393, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 507, 671-710 and 755-777.

In some aspects, a polynucleotide provided herein encodes a polypeptide having at least 80%, at least 85%, at least 90%, at least 91%, at least 92%, at least 93%, at least 94%, at least 95%, at least 96%, at least 97%, at least 98%, at least 99% or 100% identity to one or more of SEQ ID NOs:46-113, 150-165, 190-258, 291, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505 and 711-754. In some aspects, a polynucleotide provided herein encodes a polypeptide having at least 80%, identity to one or more of SEQ ID NOs:46-113, 150-165, 190-258, 291, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505 and 711-754. In some aspects, a polynucleotide provided herein encodes a polypeptide having at least 85%, identity to one or more of SEQ ID NOs:46-113, 150-165, 190-258, 291, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505 and 711-754. In some aspects, a polynucleotide provided herein encodes a polypeptide having at least 90%, identity to one or more of SEQ ID NOs:46-113, 150-165, 190-258, 291, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505 and 711-754. In some aspects, a polynucleotide provided herein encodes a polypeptide having at least 91%, identity to one or more of SEQ ID NOs:46-113, 150-165, 190-258, 291, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505 and 711-754. In some aspects, a polynucleotide provided herein encodes a polypeptide having at least 92%, identity to one or more of SEQ ID NOs:46-113, 150-165, 190-258, 291, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505 and 711-754. In some aspects, a polynucleotide provided herein encodes a polypeptide having at least 93%, identity to one or more of SEQ ID NOs:46-113, 150-165, 190-258, 291, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505 and 711-754. In some aspects, a polynucleotide provided herein encodes a polypeptide having at least 94%, identity to one or more of SEQ ID NOs:46-113, 150-165, 190-258, 291, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505 and 711-754. In some aspects, a polynucleotide provided herein encodes a polypeptide having at least 95%, identity to one or more of SEQ ID NOs:46-113, 150-165, 190-258, 291, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505 and 711-754. In some aspects, a polynucleotide provided herein encodes a polypeptide having at least 96%, identity to one or more of SEQ ID NOs:46-113, 150-165, 190-258, 291, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505 and 711-754. In some aspects, a polynucleotide provided herein encodes a polypeptide having at least 97%, identity to one or more of SEQ ID NOs:46-113, 150-165, 190-258, 291, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505 and 711-754. In some aspects, a polynucleotide provided herein encodes a polypeptide having at least 98%, identity to one or more of SEQ ID NOs:46-113, 150-165, 190-258, 291, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505 and 711-754. In some aspects, a polynucleotide provided herein encodes a polypeptide having at least 99%, identity to one or more of SEQ ID NOs:46-113, 150-165, 190-258, 291, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505 and 711-754. In some aspects, a polynucleotide provided herein encodes the polypeptide of one or more of SEQ ID NOs:46-113, 150-165, 190-258, 291, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505 and 711-754.

In some aspects, provided herein is a vector comprising a polynucleotide provided herein.

In some aspects, provided herein is a host cell comprising a polynucleotide or vector provided herein. As used herein, the term “host cell” can be any type of cell, e.g., a primary cell, a cell in culture, or a cell from a cell line. In some aspects, the term “host cell” refers to a cell containing a foreign gene [e.g., a cell subjected to gene delivery or transfected with a polynucleotide (e.g., DNA or mRNA) encoding the gene] transfected with a polynucleotide and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome.

Methods which are well known to those skilled in the art can be used to construct vectors encoding antigen binding polypeptides and polypeptide complexes (e.g., CDR, VH, VL, heavy chain and/or light chain coding sequences and appropriate transcriptional and translational control signals). These methods include, for example, in vitro recombinant DNA techniques, synthetic techniques, and in vivo genetic recombination.

A vector can be transferred to a host cell by conventional techniques and the resulting cell can then be cultured by conventional techniques to produce an antigen binding polypeptide or antigen binding polypeptide complex comprising, e.g., six CDRs, VH, VL, VH and VL, heavy chain, light chain, or heavy and light chain, or a domain thereof (e.g., one or more CDRs, VH, VL, VH and VL, heavy chain, or light chain). Thus, provided herein are host cells containing a polynucleotide encoding an antigen binding polypeptide or polypeptide complex comprising, e.g., comprising six CDRs, VH, VL, VH and VL, heavy chain, light chain, or heavy and light chain, or a domain thereof (e.g., one or more CDRs, VH, VL, VH and VL, heavy chain, or light chain), operably linked to a promoter for expression of such sequences in the host cell. In some aspects, vectors encoding both heavy and light chains, or a domain thereof, individually, can be co-expressed in the host cell for expression. In some aspects, a host cell contains a vector comprising a polynucleotide encoding both a heavy chain and light chain, or a domain thereof. In some aspects, a host cell contains two different vectors, a first vector comprising a polynucleotide encoding a heavy chain or a domain thereof, and a second vector comprising a polynucleotide encoding a light chain or a domain thereof. In some aspects, a first host cell comprises a first vector comprising a polynucleotide encoding a heavy chain or a domain thereof, and a second host cell comprises a second vector comprising a polynucleotide encoding a light chain or a domain thereof. In some aspects, provided herein is a population of host cells comprising such a first host cell and such a second host cell.

In some aspects, provided herein is a population of vectors comprising a first vector comprising a polynucleotide encoding a light chain or domain thereof, and a second vector comprising a polynucleotide encoding a heavy chain or domain thereof. Alternatively, a single vector can be used which encodes, and is capable of expressing, both heavy and light chain polypeptides or a domain thereof.

A variety of host-vector systems can be utilized to express the polypeptides and polypeptide complexes provided herein. Such host-vector systems represent vehicles by which the coding sequences of interest can be produced and subsequently purified, but also represent cells which can, when transformed or transfected with the appropriate nucleotide coding sequences, express a polypeptide or polypeptide complex provided herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS (e.g., COS1 or COS), CHO, BHK, MDCK, HEK 293, NS0, PER.C6, VERO, CRL7030, HsS78Bst, HeLa, and NIH 3T3, HEK-293T, HepG2, SP210, R1.1, B-W, L-M, BSC1, BSC40, YB/20, and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In some aspects, cells for expressing polypeptide or polypeptide complexes described herein are CHO cells, for example CHO cells from the CHO GS System™ (Lonza). In some aspects, cells for expressing the polypeptides or polypeptide complexes provided herein are human cells, e.g., human cell lines. In some aspects, a mammalian expression vector is pOptiVEC™ or pcDNA3.3. In some aspects, bacterial cells such as Escherichia coli, or eukaryotic cells (e.g., mammalian cells) are used for the expression of recombinant polypeptides. For example, mammalian cells such as Chinese hamster ovary (CHO) cells in conjunction with a vector such as the major intermediate early gene promoter element from human cytomegalovirus is an effective expression system for polypeptides (Foecking M K & Hofstetter H (1986) Gene 45: 101-105; and Cockett M I et al., (1990) Biotechnology 8: 662-667). In some aspects, the polypeptides or polypeptide complexes provided herein are produced by HEK-293T cells.

In addition, a host cell strain can be chosen which modulates the expression of the inserted sequences, or modifies and processes the gene product in the specific fashion desired. Such modifications (e.g., glycosylation) and processing (e.g., cleavage) of protein products can contribute to the function of the protein. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT20 and T47D, NS0 (a murine myeloma cell line that does not endogenously produce any immunoglobulin chains), CRL7030, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells.

Once a polypeptide or polypeptide complex provided herein has been produced by recombinant expression, it can be purified by any method known in the art for purification of a protein or immunoglobulin molecule, for example, by chromatography (e.g., ion exchange, affinity, particularly by affinity for the specific antigen after Protein A, and size exclusion chromatography), centrifugation, differential solubility, or by any other standard technique for the purification of proteins. Further, the polypeptides or polypeptide complexes provided herein can be fused to heterologous polypeptide sequences provided herein (e.g., peptide tags) or otherwise known in the art to facilitate purification.

In some aspects, a polypeptide or polypeptide complex provided herein is isolated or purified. Generally, an isolated polypeptide or polypeptide complex is one that is substantially free of other polypeptides or polypeptide complexes with different antigenic specificities. For example, in some aspects, a preparation of a polypeptide or polypeptide complex described herein is substantially free of cellular material and/or chemical precursors.

### Pharmaceutical Compositions and Kits

In some aspects, provided herein is a pharmaceutical composition comprising an antigen binding polypeptide, antigen binding polypeptide complex (e.g., antibody or antigen binding fragment thereof), polypeptide, polynucleotide, vector, or host cell provided herein.

In some aspects, a pharmaceutical composition provided herein comprises (1) an antigen binding polypeptide, antigen binding polypeptide complex (e.g., antibody or antigen binding fragment thereof), polypeptide, polynucleotide, vector, or host cell provided herein, and (2) a pharmaceutically acceptable carrier. The term “pharmaceutically acceptable carrier” includes any and all solvents, co-solvents, complexing agents, dispersion media, coatings, antibacterial and antifungal agents, isotonic and absorption delaying agents, and the like, which are not biologically or otherwise undesirable. The use of such media and agents for pharmaceutically active substances is known in the art. Except insofar as any conventional media or agent is incompatible with the active ingredient, its use in the therapeutic formulations is contemplated. Supplementary active ingredients can also be incorporated into the pharmaceutical compositions provided herein. In addition, various excipients, such as are commonly used in the art, can be included. These and other such compounds are described in the literature, e.g., in the Merck Index, Merck & Company, Rahway, NJ. Considerations for the inclusion of various components in pharmaceutical compositions are described, e.g., in Gilman et al. (Eds.) (2010); Goodman and Gilman's: The Pharmacological Basis of Therapeutics, 12th Ed., The McGraw-Hill Companies. In some aspects, the pharmaceutical composition is for parenteral, intravenous or subcutaneous administration.

In some aspects, provided herein is a pharmaceutical composition comprising one or more antigen binding polypeptides or antigen binding polypeptide complexes provided herein (e.g., 1, 2, 3, 4, 5 or more). In some aspects, the pharmaceutical composition further comprises a pharmaceutically acceptable carrier. In some aspects, one or more of the antigen binding polypeptides or antigen binding polypeptide complexes is an antibody or antigen binding fragment thereof provided herein. In some aspects, the antigen binding polypeptide complex is MX828, MX829, MX830, MX831, MX834, MX837, MX835, MX838, MX1042 or MX1069.

In some embodiments, the present invention comprises a combination of a monoclonal antibody(ies) and an antigen binding polypeptide or antigen binding polypeptide complex which comprises a multispecific antibody.

In some aspects, the present invention comprises a nucleic acid encoding an antigen binding polypeptide or antigen binding polypeptide complex for in vivo administration. In some aspects, the nucleic acid encoding an antigen binding polypeptide or antigen binding polypeptide complex is a stabilized nucleic acid selected from a stabilized mRNA.

In some aspects, the present disclosure relates to one or more mRNA that encodes an antigen binding polypeptide or antigen binding polypeptide complex (e.g., antibody or antigen binding fragment thereof). Examples of such mRNA include, but are not limited to, any one of SEQ ID NOs:695-702.

In some aspects, a nucleic acid encoding an antigen binding polypeptide or antigen binding polypeptide complex comprises said nucleic acid or mRNA and a carrier to form a pharmaceutical composition.

In some aspects, a pharmaceutical composition provided herein comprises a lipid nanoparticle composition. As used herein, a “lipid nanoparticle composition,” “lipid nanoparticle formulation,” “LNP composition,” or “LNP formulation” is a composition comprising one or more lipids. The lipids in a lipid nanoparticle composition are typically sized on the order of micrometers or smaller. In some aspects, the lipids in a lipid nanoparticle composition have an average size of less than 1 micrometer.

In some aspects, the pharmaceutical composition comprises (1) one or more polynucleotides encoding an antigen binding polypeptide or antigen binding polypeptide complex provided herein, and (2) a lipid nanoparticle composition. In some aspects, the lipid nanoparticle composition comprises one or more of (i) a cationic and/or ionizable lipid, (ii) a structural lipid, (iii) a PEGylated lipid, and (iv) a phospholipid.

As used herein, a “cationic and/or ionizable lipid” is a lipid that has a positive or partial positive charge at physiological pH. Examples of a cationic and/or ionizable lipid include, but are not limited to, a lipid including a cyclic amine group, 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(33)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3P)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), (2S)-2-({8-[(30)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)), and mixtures thereof.

As used herein, a “structural lipid” is a fat synthesized from mixtures of long-chain and medium-chain fatty acids. A structural lipid is typically a triacylglycerol restructured or modified to change the fatty acid composition and/or positional distribution. Examples of a structural lipid include, but are not limited to, cholesterol, fecosterol, sitosterol, ergosterol, campesterol, stigmasterol, brassicasterol, tomatidine, tomatine, ursolic acid, alpha-tocopherol, and mixtures thereof.

As used herein, a “PEGylated lipid” is a lipid that has been modified to include polyethylene glycol or “PEG.” Examples of a PEGylated lipid include, but are not limited to, PEG-modified phosphatidylethanolamines, PEG-modified phosphatidic acids, PEG-modified ceramides, PEG-modified dialkylamines, PEG-modified diacylglycerols, PEG-modified dialkylglycerols, PEG-c-DOMG, PEG-DMG, PEG-DLPE, PEG-DMPE, PEG-DPPC, a PEG-DSPE lipid, and mixtures thereof.

As used herein, a “phospholipid” is a class of lipids whose molecule has a hydrophilic “head” containing a phosphate group and two hydrophobic “tails” derived from fatty acids, joined by an alcohol residue (usually a glycerol molecule). Examples of a phospholipid include, but are not limited to, 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof. In some aspects, the phospholipid is DSPC. In some aspects, the phospholipid is DOPE. In some aspects, the phospholipid is DSPC and DOPE.

Exemplary lipid nanoparticle compositions and methods for making them are described, for example, herein and in U.S. Pat. Nos. 11,524,023; 11,485,972; 10,898,574; 10,703,789; 10,702,600; 10,577,403; 10,442,756; 10,266,485; 10,064,959; 9,868,692; Int'l Pub. No. WO 2019/046809; Int'l Pub. No. WO 2020/160397; U.S. Pub. No. 2020/0306191; Xu et al., Adv. Nanobio. Res. 2:2, 2022; Cullis et al., Mol. Ther. 25(7):1467-1475, 2017; Fan et al., J. Pharm. Biomed. Anal. 192:113642, 2020; Paunovska et al., Nature Rev., 23:265-280, 2022; Buck et al., ACS Nano. 13:3754-3782, 2019; Pardi et al., Nature Comm. 8:14630, 2017; and Pardi et al., J. Control Release, 217:345-351, 2015, which are incorporated by reference herein. Such methods include, but are not limited to, back translating DNA coding for an antigen binding polypeptide or antigen binding polypeptide complex provided herein (e.g., an antibody or antigen binding fragment thereof) into an in vitro transcription (IVT) plasmid according to such published methods.

Exemplary compositions and methods related to mRNA preparation and delivery are described, for example, herein and in Int'l Pub. No. WO 2018/081638, Int'l Pub. No. WO 2017/182524, U.S. Pat. No. 9,012,219, Int'l Pub. No. WO 2016/176330, U.S. Pat. No. 9,371,511, U.S. Appl. Pub. No. 2018/0028645, U.S. Appl. Pub. No. 23018/0344838, U.S. Pub. No. 2018/0265848, U.S. Appl. Pub. No. 2017/0043037, U.S. Appl. Pub. No. 2017/0327842, U.S. Pat. No. 10,006,007, U.S. Appl. Pub. No. 2013/0261172, U.S. Appl. Pub. No. 2013/0197068, U.S. Appl. Pub. No. 2015/0038558, U.S. Appl. Pub. No. 2016/0032316, U.S. Appl. Pub. No. 2013/0111615, U.S. Appl. Pub. No. 2009/0286852, Cullis et al., Mol. Ther. 25(7):1467-1475, 2017; Pardi et al., Nature Comm. 8:14630, 2017; Pardi et al., J. Control Release, 217:345-351, 2015; and Grier et al., Mol. Ther. Nucleic Acids, 5:e306 (2016), which are incorporated by reference herein. Such methods include, but are not limited to, in vitro transcription (e.g., using PCR products or a linearized plasmid); IVT vectors; and synthesizing mRNA using, e.g., HiScribe T7 High Yield RNA Synthesis Kit (New England Biolabs), incorporating, e.g., Pseudouridine-5′-Triphosphate (TriLink BioTechnologies). In some aspects, 5′ capped mRNA can also be generated during this process using, e.g., CleanCap Reagent AG (TriLink BioTechnologies) to form, e.g., 100 bp or 120 bp poly(A) tails.

In some aspects, provided herein is a pharmaceutical composition comprising one or more antigen binding polypeptides or antigen binding polypeptide complexes provided herein (e.g., 1, 2, 3, 4, 5 or more) and an additional pharmaceutical agent. In some aspects, the pharmaceutical composition further comprises a pharmaceutically acceptable carrier. In some aspects, one or more of the antigen binding polypeptides or antigen binding polypeptide complexes is an antibody or antigen binding fragment thereof provided herein. In some aspects, the antigen binding polypeptide complex is MX828, MX829, MX830, MX831, MX834, MX837, MX835, MX838, MX1042 or MX1069.

In some aspects, provided herein is a pharmaceutical composition comprising a combination of antigen binding polypeptides or antigen binding polypeptide complexes provided herein. In some aspects, the pharmaceutical composition comprises two or more of MX828, MX829, MX830, MX831, MX834, MX837, MX835, MX838, MX1042 and MX1069. In some aspects, the pharmaceutical composition comprises MX828 and MX829. In some aspects, the pharmaceutical composition comprises MX830 and MX831.

In some aspects, provided herein is a pharmaceutical composition comprising an antigen binding polypeptide or antigen binding polypeptide complex provided herein and an additional pharmaceutical agent. In some aspects, the antigen binding polypeptide complex is MX828, MX829, MX830, MX831, MX834, MX837, MX835, MX838, MX1042, MX1069, or a combination thereof.

In some aspects, the additional pharmaceutical agent is 25-hydroxyvitamin D, an agent that potentiates vitamin D action, an anti-viral agent, an anti-malarial agent, an antibiotic, or a combination thereof.

In some aspects, the additional pharmaceutical agent is 25-hydroxyvitamin D.

In some aspects, the agent that potentiates vitamin D action is a CYP24 inhibitor, 1,25-dihydroxyvitamin D compound, or a combination thereof.

In some aspects, the anti-viral agent is an anti-retroviral agent, an antibody against SARS-CoV-2 virus, an inhibitor of reverse transcriptase, or a combination thereof.

In some aspects, the anti-viral agent is maraviroc, enfuvirtide, amantadine, lamivudine, nevirapine, efavirenz, dolutegravir, elvitegravir, raltegravir, acyclovir and any nucleoside analog of aciclovir, ganciclovir, cidofovir, forcarnet, ribavirin, interferon alpha, pegylated interferon alpha, boceprevir, atazanavir, darunavir, indinavir, oseltamivir, zanamivir, rimantadine, peremivir, valaciclovir, penciclovir, valganciclovir, foscarnet, tenofovir, adefovir, entecavir, lamivudine, telbivudine, ribavirin, glecaprevir, grazoprevir, paritaprevir, simeprevir, voxilaprevir, daclatasvir, elbasvir, ledipasvir, ombitasvir, pibrentasvir, velpatasvir, dasabuvir, famciclovir, remdesivir, trifluridine, sofobuvir, bebtelovimab (LY1404, LY-CoV1404, LY3853133), or a combination thereof.

In some aspects, the anti-viral agent is bebtelovimab.

In some aspects, the anti-viral agent is Retrovir® (3′-azido-3′-deoxypyrimidine, zidovudine), 3′-azido-3′-deoxythymidine (AZT), HMD® (2′,3′-dideoxycytidine, zalcitabine), VidexEC® (2′,3′dideoxyinosine, didanosine), Epivir® (lamivudine), Zerit® (stavudine), Viread® (tenofovir DF), Ziagen® (abacavir), Emtriva® (emtricitabine, FTC), Rescriptor® (delavirdine), Sustiva® (efavirenz), Viramune® (nevirapine, 11-cyclopropyl-4-methyl-5,11-dihydro-6H-dipyrido[3,2-b:2′,3′-e][1,4]diazepin-6-one), trisodium phosphonoformate, ammonium-21-tungstenato-9-antimonate, 1-β-D-ribofuranoxyl-1,2,4-triazole-3-carboxamide, Aganerase® (amprenavir), Reyataz® (atazanavir), Lexiva® (fosamprenavir), Crixivan® (indinavir), Viracept® (nelfinavir), Norvir® (ritonavir), Fortovase® or Invirase® (saquinavir), lasinavir (5(S)-(tert-butoxycarbonylamino)-4(S)-hydroxy-6-phenyl-2(R)(2,3,4-trimethoxyphenylmethyl)-hexanoyl-(L)-valyl-N-(2-metoxy-ethyl)-amide), adriamycin, KVX-478, VX-478, 141W94, AG-1343, KNI-272, U-96988, BILA-2011 BS (palinavir), polymannoacetate, Fuzeon® (enfuvirtide, T-20), Epzicom® (abacavir and lamivudine), Trizivir® (abacavir, lamivudine and zidovudine), Truvada® (emtricitabine and tenofir DF), Combivir® (lamivudine and zidovudine), Kaletra® (lopinavir and ritonavir), bebtelovimab, or a combination thereof.

In some aspects, the pharmaceutical composition comprises bebtelovimab and one or more of MX828, MX829, MX830, MX831, MX834, MX837, MX835, MX838, MX1042 or MX1069. In some aspects, the pharmaceutical composition comprises bebtelovimab and MX828. In some aspects, the pharmaceutical composition comprises bebtelovimab and MX830. In some aspects, the pharmaceutical composition comprises bebtelovimab, MX828 and MX829. In some aspects, the pharmaceutical composition comprises bebtelovimab, MX830 and MX831.

In some aspects, a pharmaceutical composition provided herein further comprises a pharmaceutically acceptable carrier.

In some aspects, provided herein is a kit comprising an antigen binding polypeptide, antigen binding polypeptide complex (e.g., antibody or antigen binding fragment thereof), polypeptide, polynucleotide, vector, host cell, or pharmaceutical composition provided herein, or a combination thereof. Once a pharmaceutical composition has been formulated, it can be stored in sterile vials as a solution, suspension, gel, emulsion, solid, crystal, or as a dehydrated or lyophilized powder. Such formulations may be stored either in a ready-to-use form or in a form (e.g., lyophilized) that is reconstituted prior to administration. In some aspects, provided herein is a kit for producing a single-dose administration unit. In some aspects, the kit contains a first container having a dried antigen binding polypeptide or polypeptide complex provided herein (e.g., antibody or antigen binding fragment thereof) and a second container having an aqueous formulation. In some aspects, provided herein is a kit containing an antigen binding polypeptide or polypeptide complex provided herein (e.g., antibody or antigen binding fragment thereof) in a single and/or multi-chambered pre-filled syringe (e.g., liquid syringe and/or lyosyringe). In some aspects, the kit further contains components for intravenous or subcutaneous administration.

In some aspects, a kit provided herein comprises one or more antigen binding polypeptides or antigen binding polypeptide complexes (e.g., antibodies or antigen binding fragments thereof) provided herein (e.g., 1, 2, 3, 4, 5 or more). In some aspects, a kit provided herein comprises an antigen binding polypeptide or antigen binding polypeptide complexes (e.g., antibodies or antigen binding fragments thereof) provided herein.

In some aspects, a kit provided herein comprises one or more pharmaceutical compositions comprising an antigen binding polypeptide complex (e.g., antibody or antigen binding fragment thereof) provided herein (e.g., 1, 2, 3, 4, 5 or more). In some aspects, a kit provided herein comprises a pharmaceutical compositions comprising an antigen binding polypeptide complexes (e.g., antibody or antigen binding fragment thereof) provided herein. In some aspects, the pharmaceutical composition further comprises a pharmaceutically acceptable carrier.

In some aspects, a kit provided herein comprises (i) one or more antigen binding polypeptide complexes (e.g., antibodies or antigen binding fragments thereof) provided herein (e.g., 1, 2, 3, 4, 5 or more), and (ii) one or more additional pharmaceutical agents (e.g., 1, 2, 3, 4, 5 or more). In some aspects, a kit provided herein comprises (i) an antigen binding polypeptide complex (e.g., antibody or antigen binding fragment thereof) provided herein, and (ii) an additional pharmaceutical agent.

In some aspects, a kit provided herein comprises (i) one or more pharmaceutical compositions comprising an antigen binding polypeptide complex (e.g., antibody or antigen binding fragment thereof) provided herein (e.g., 1, 2, 3, 4, 5 or more), and (ii) one or more additional pharmaceutical agents (e.g., 1, 2, 3, 4, 5 or more). In some aspects, a kit provided herein comprises (i) a pharmaceutical composition comprising an antigen binding polypeptide complex (e.g., antibody or antigen binding fragment thereof) provided herein, and (ii) an additional pharmaceutical agent. In some aspects, the pharmaceutical composition further comprises a pharmaceutically acceptable carrier.

In some aspects, the kit further comprises instructions for use, for example, for any of the methods of use described herein (e.g., treating or preventing a SARS-CoV-2 infection).

In some aspects, the antigen binding polypeptide complex is MX828, MX829, MX830, MX831, MX834, MX837, MX835, MX838, MX1042 or MX1069, and the additional pharmaceutical agent is bebtelovimab. In other aspects, the antigen binding polypeptide complex is selected from any one of those listed in Table 1.

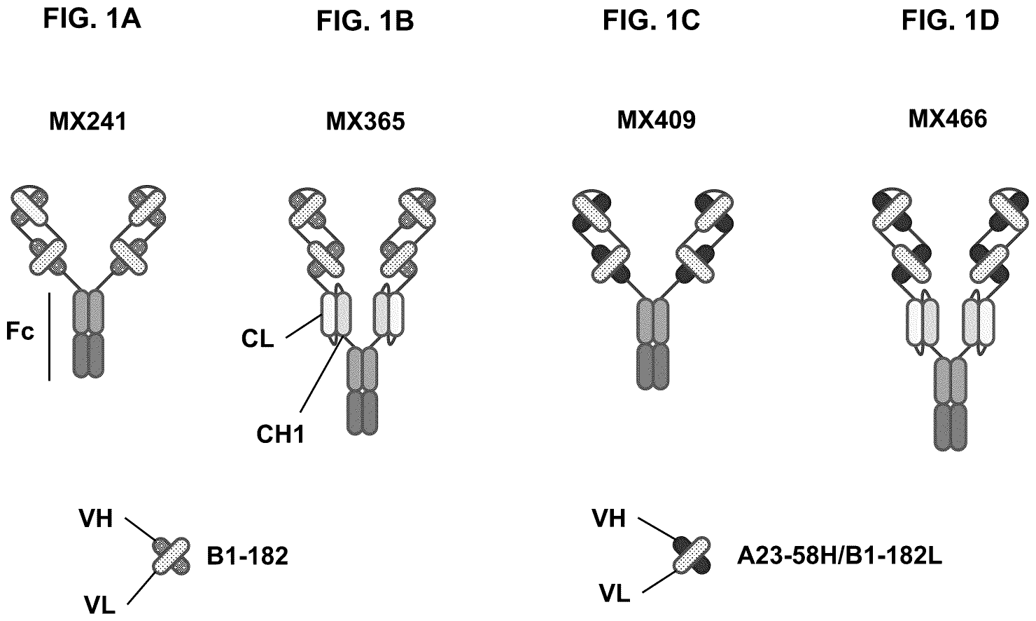

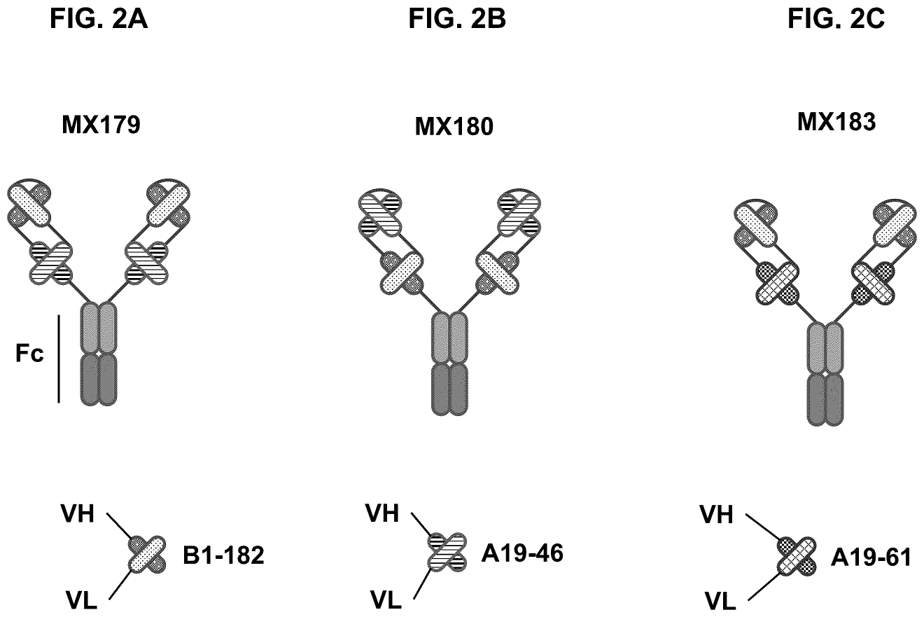

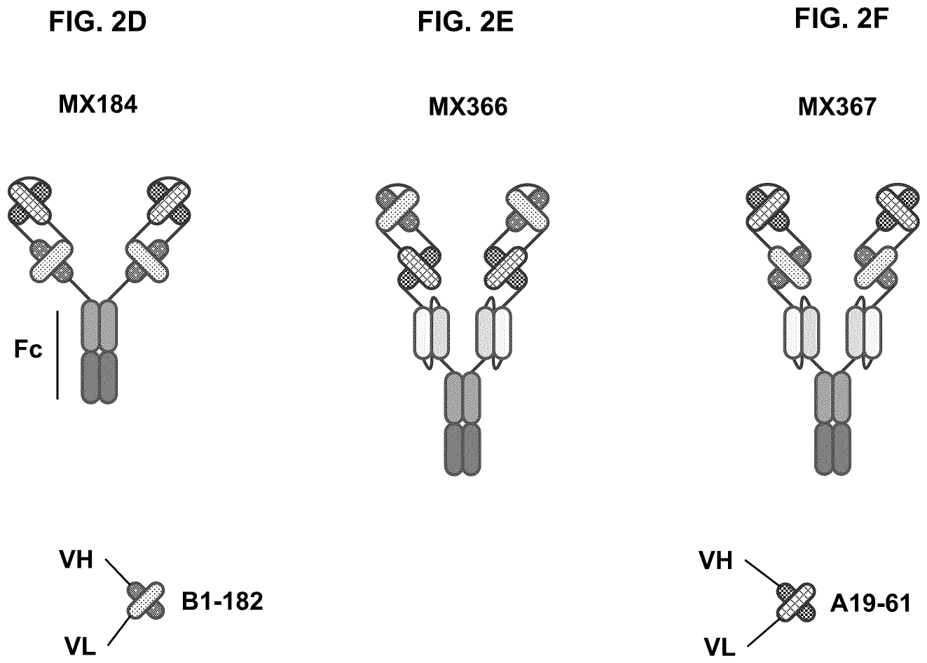

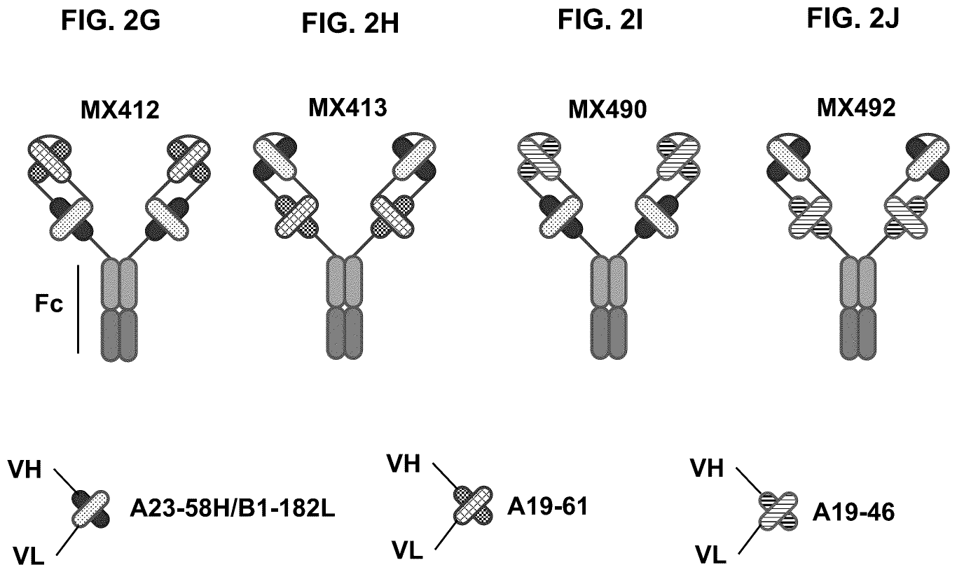

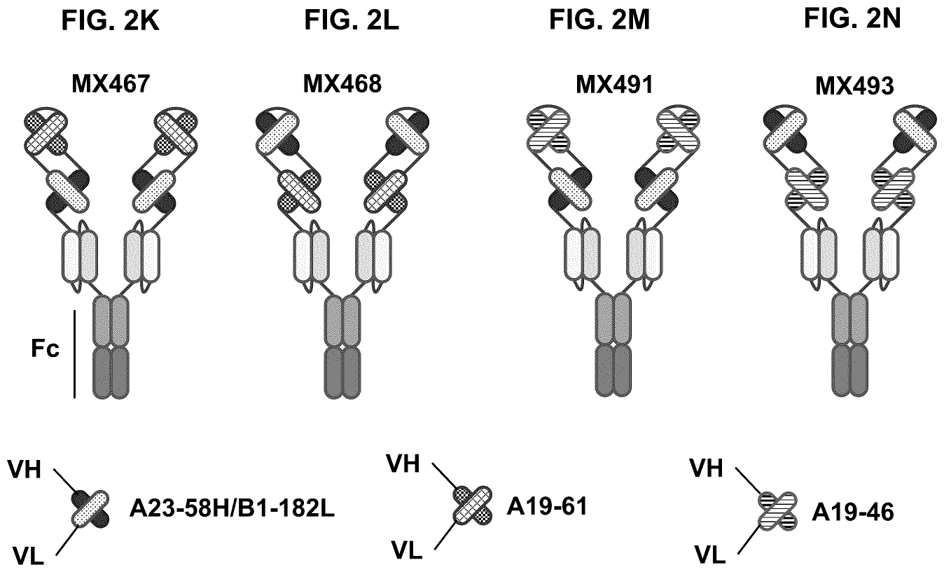

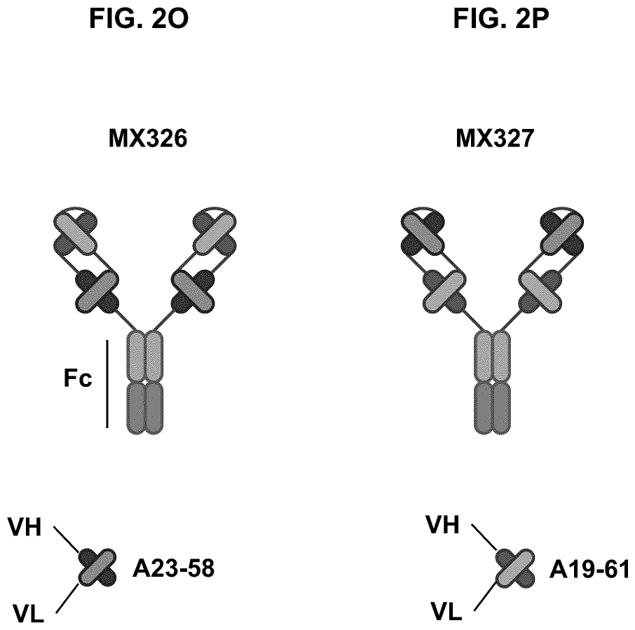

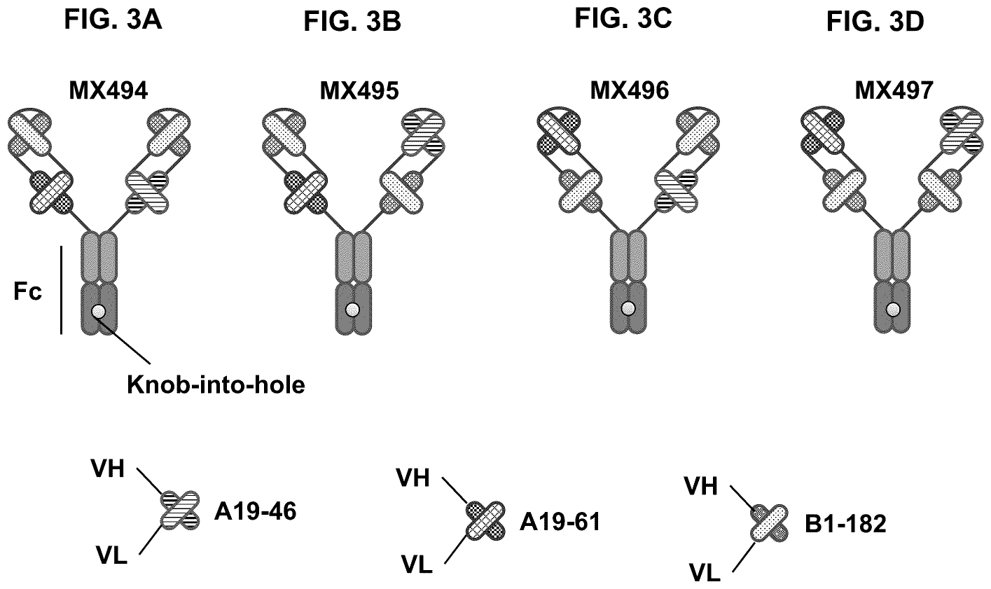

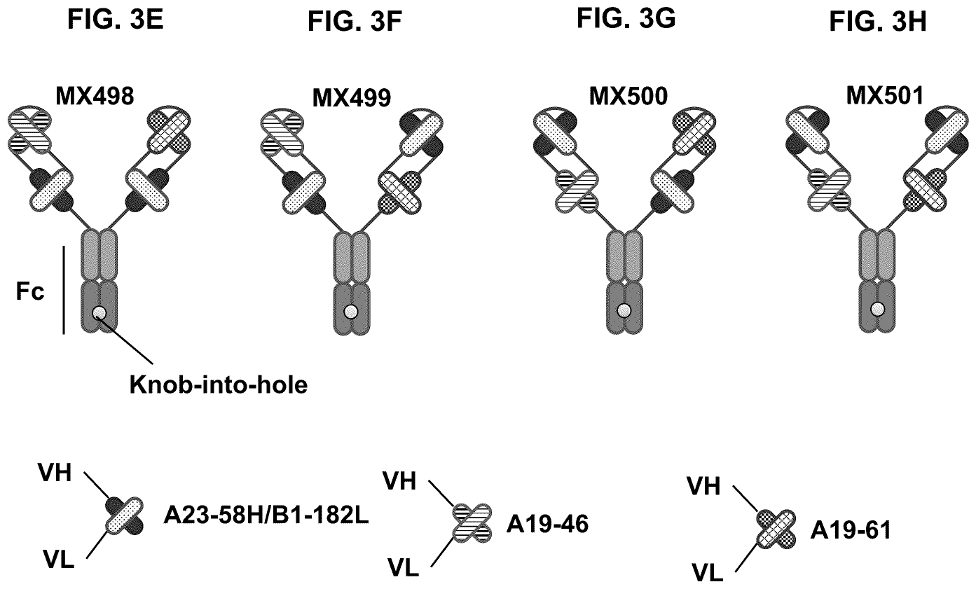

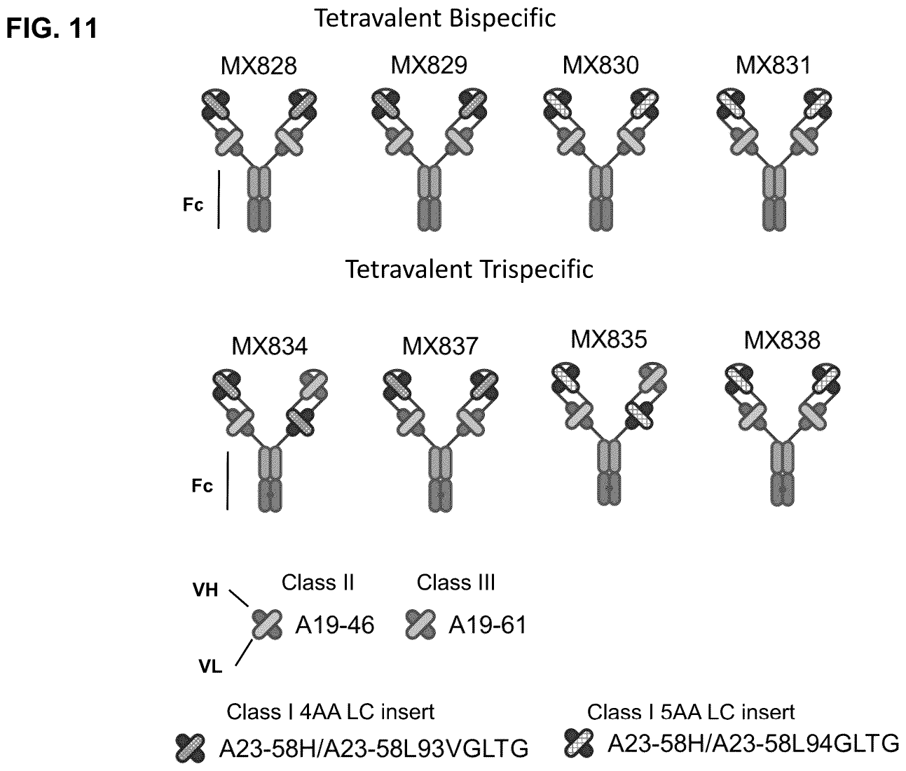

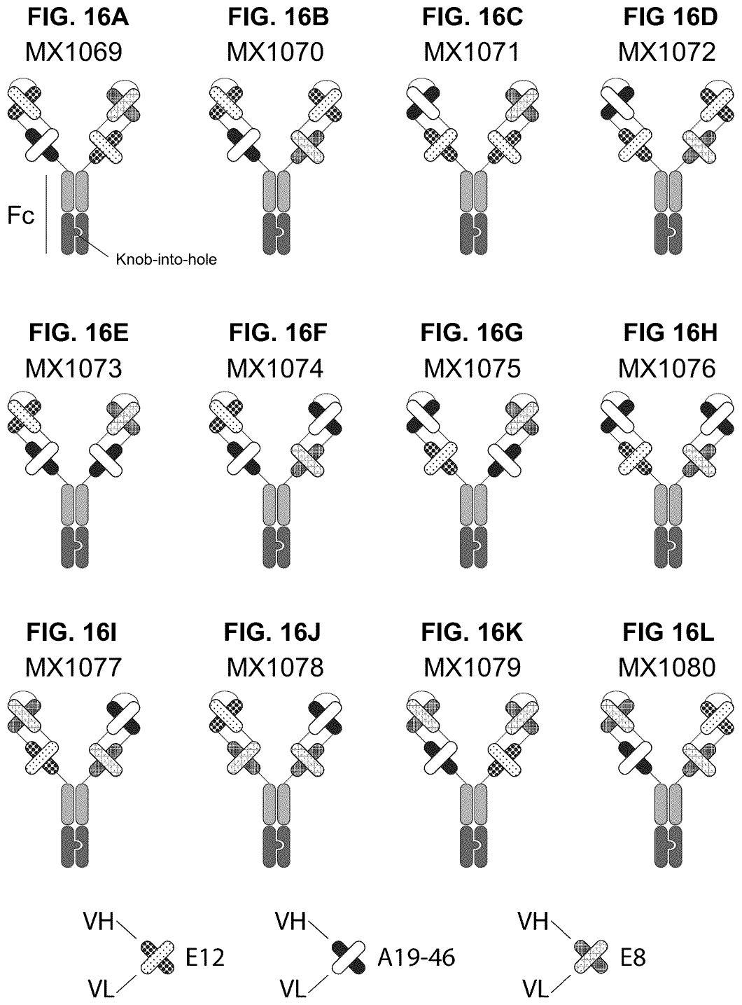

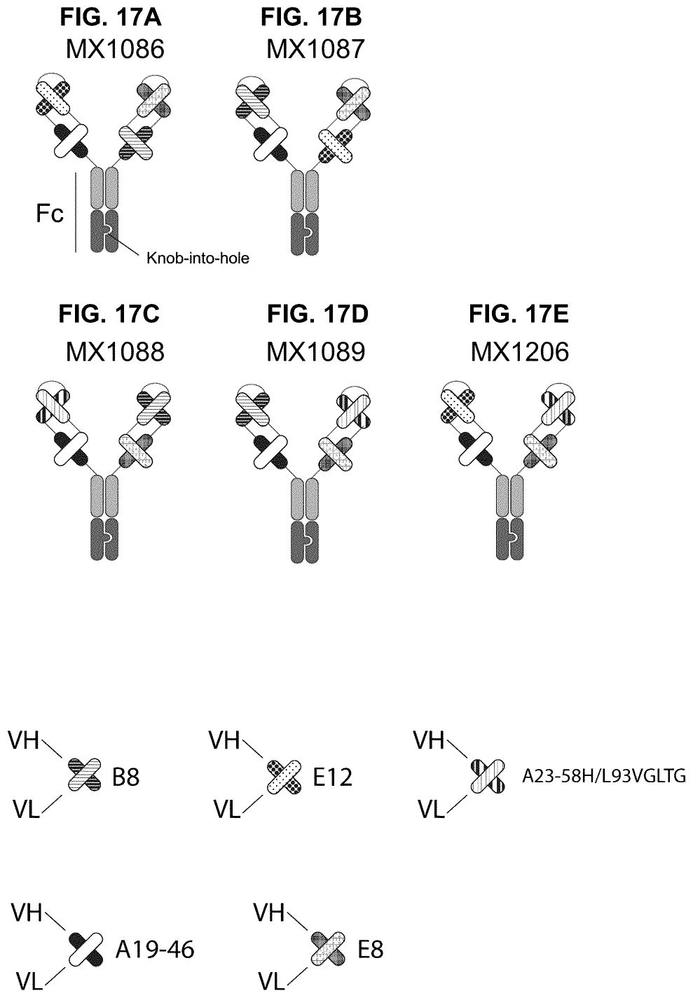

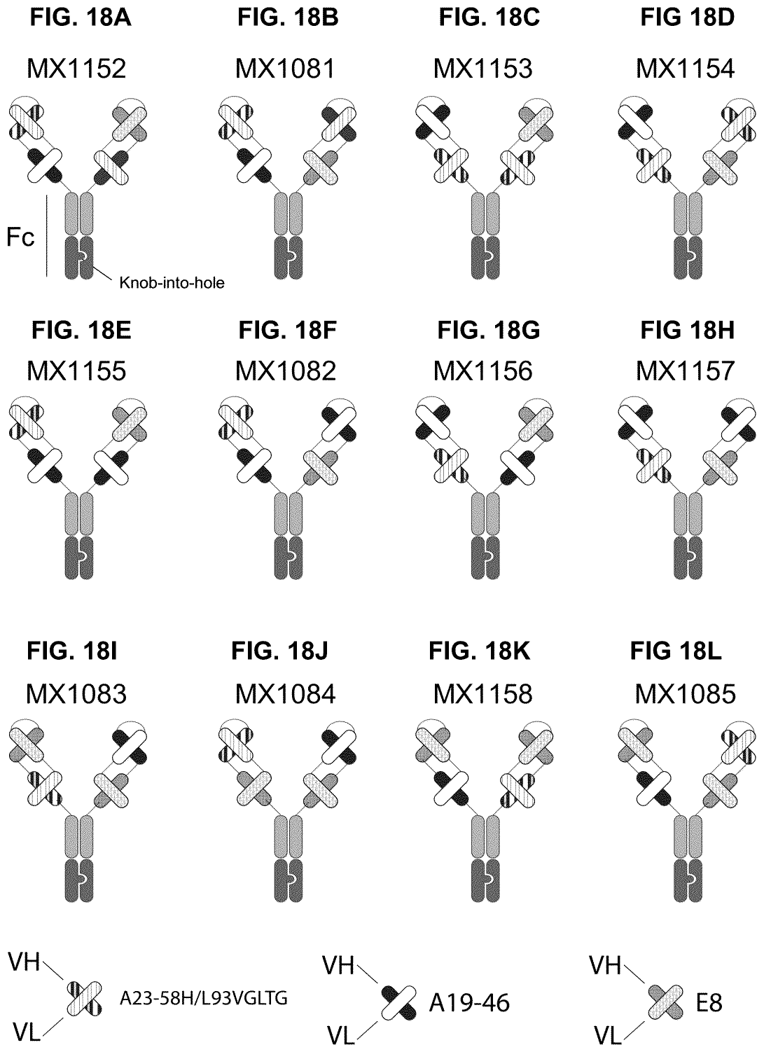

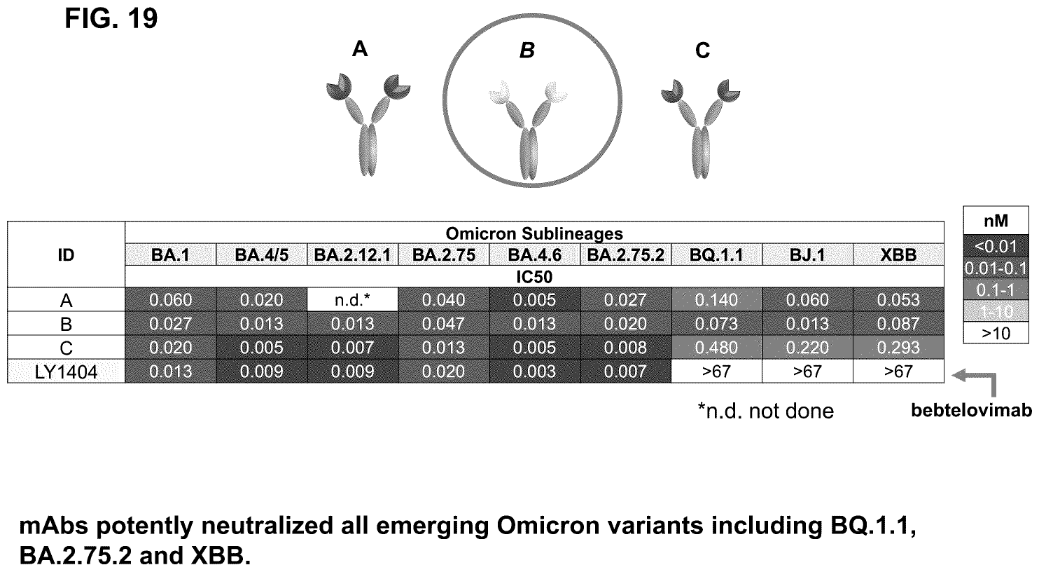

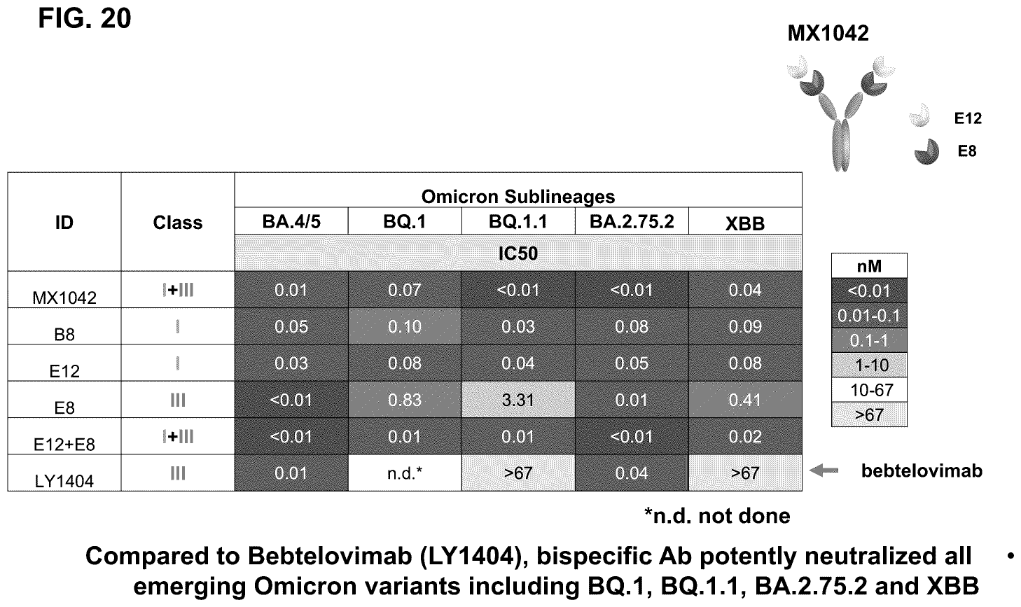

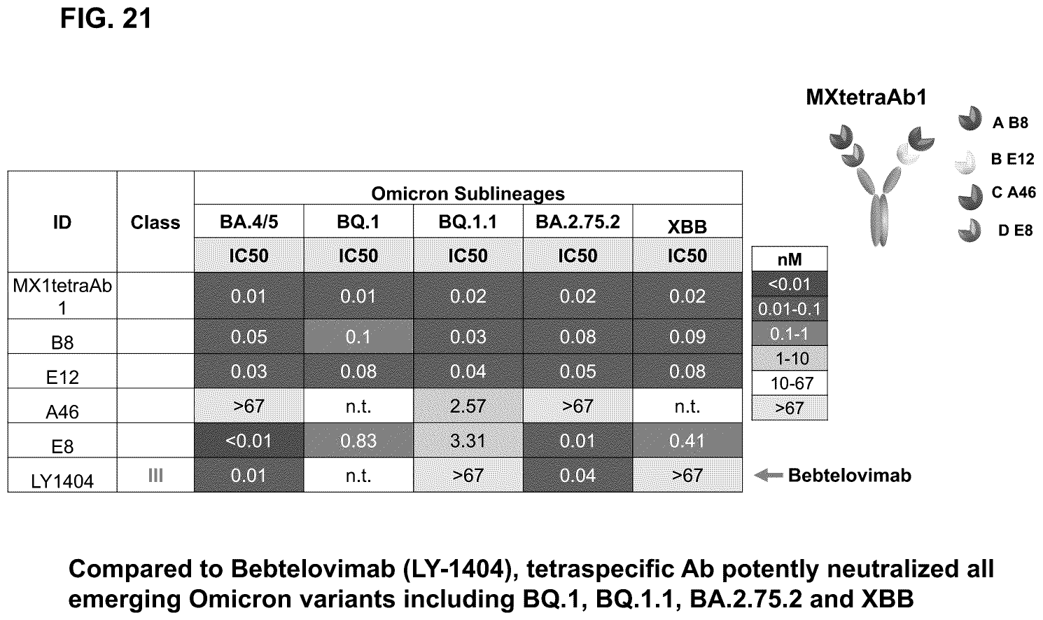

TABLE 1

| MX Number | FIG. | VH/VL pairs | msAb SEQ ID NO: |
|---|---|---|---|
| MX241 | FIG. 1A | B1-182 | 78, 78 |
| MX365 | FIG. 1B | B1-182 | 79, 79 |
| MX180 | FIG. 2B | B1-182.A19-46 | 83, 83 |
| MX183 | FIG. 2C | A19-61, B1-182 | 84, 84 |
| MX179 | FIG. 2A | A19-46, B1-182 | 82, 82 |
| MX184 | FIG. 2D | B1-182, A19-61 | 85, 85 |
| MX366 | FIG. 2E | A19-61, B1-182 | 88, 88 |
| MX367 | FIG. 2F | B1-182, A19-61 | 89, 89 |
| MX413 | FIG. 2H | A19-61, A23-58H/B1-182L | 91, 91 |
| MX490 | FIG. 21 | A23-58H/B1-182L, A19-46 | 94, 94 |
| MX492 | FIG. 2J | A19-46, A23-58H/B1-182L | 96, 96 |
| MX412 | FIG. 2G | A23-58H/B1-182L, A19-61 | 90, 90 |
| MX467 | FIG. 2K | A23-58H/B1-182L, A19-61 | 92, 92 |
| MX468 | FIG. 2L | A19-61, A23-58H/B1-182L | 93, 93 |
| MX491 | FIG. 2M | A23-58H/B1-182L, A19-46 | 95, 95 |
| MX493 | FIG. 2N | A19-46, A23-58H/B1-182L | 97, 97 |
| MX326 | FIG. 20 | A19-61, A23-58 | 86, 86 |
| MX327 | FIG. 2P | A23-58, A19-61 | 87, 87 |
| MX494 | FIG. 3A | A19-46, A19-61, B1-182, B1-182 | 98, 99 |
| MX495 | FIG. 3B | B1-182, A19-61, B1-182, A19-46 | 100, 101 |
| MX496 | FIG. 3C | A19-46, B1-182, A19-61, B1-182 | 102, 103 |
| MX497 | FIG. 3D | B1-182, B1-182, A19-61, A19-46 | 104, 105 |
| MX498 | FIG. 3E | A23-58H/B1-182L, A23-58H/B1-182L, | 106, 107 |
| | | A19-46, A19-61 | |
| MX499 | FIG. 3F | A19-61, A23-58H/B1-182L, A19-46, | 108, 109 |
| | | A23-58H/B1-182L | |
| MX500 | FIG. 3G | A23-58H/B1-182L, A19-46, A23- | 110, 111 |
| | | 58H/B1-182L, A19-61 | |
| MX501 | FIG. 3H | A19-61, A19-46, A23-58H/B1-182L, | 112, 113 |
| | | A23-58H/B1-182L | |
| MX828 | FIG. 11 | A19-46, A19-46, A23-58H/A23- | 190, 190 |
| | | 58L93VGLTG, A23-58H/A23- | |
| | | 58L93VGLTG | |
| MX829 | FIG. 11 | A19-61, A19-61, A23-58H/A23- | 191, 191 |
| | | 58L93VGLTG, A23-58H/A23- | |
| | | 58L93VGLTG | |
| MX830 | FIG. 11 | A19-46, A19-46, A23-58H/A23- | 192, 192 |
| | | 58L94GLTG, A23-58H/A23- | |
| | | 58L94GLTG | |
| MX831 | FIG. 11 | A19-61, A19-61, A23-58H/A23- | 193, 193 |
| | | 58L94LGTG, A23-58H/A23- | |
| | | 58L94GLTG | |
| MX834 | FIG. 11 | A23-58H/A23-58L93VGLTG, A19-46, | 194, 195 |
| | | A23-58H/A23-58L93VGLTG, A19-61 | |
| MX837 | FIG. 11 | A19-61, A19-46, A23-58H/A23- | 196, 197 |
| | | 58L93VGLTG, A23-58H/A23- | |
| | | 58L93VGLTG | |
| MX835 | FIG. 11 | A23-58H/A23-58L94GLTG, A19-46, | 198, 199 |
| | | A23-58H/A23-58L94GLTG, A19-61 | |
| MX838 | FIG. 11 | A19-61, A19-46, A23-58H/A23- | 200, 201 |
| | | 58L94GLTG, A23-58H/A23- | |
| | | 58L94gLTG | |
| MX1019 | FIG. 17A | B8, B8 | 329, 330 |
| MX1025 | FIG. 17B | E12, E12 | 331, 332 |
| MX1026 | FIG. 17C | E8, E8 | 333, 334 |
| MX1018 | FIG. 17D | B8, B8, B8, B8 | 291, 292 |
| MX1029 | FIG. 17E | E12, E12, E12, E12 | 295, 295 |
| MX1020 | FIG. 17F | E8, E8, E8, E8 | 297, 297 |
| MX1021 | FIG. 18A | A19-46, A19-46, B8, B8 | 299, 299 |
| MX1022 | FIG. 18B | E8, E8, B8, B8 | 301, 301 |
| MX1023 | FIG. 18C | B8, B8, A19-46, A19-46 | 303, 303 |
| MX1024 | FIG. 18D | B8, B8, E8, E8 | 305, 305 |
| MX1027 | FIG. 18E | A19-46, B8, A19-46, B8 | 307, 307 |
| MX1028 | FIG. 18F | E8, B8, E8, B8 | 311, 311 |
| MX1054 | FIG. 19A | B8, A19-46, B8, E8 | 345, 347 |
| MX1062 | FIG. 19B | E8, A19-46, B8, B8 | 349, 351 |
| MX1063 | FIG. 19C | B8, B8, A19-46, E8 | 353, 355 |
| MX1064 | FIG. 19D | E8, B8, A19-46, B8 | 357, 359 |
| MX1065 | FIG. 19E | A19-46, A19-46, B8, E8 | 361, 363 |
| MX1066 | FIG. 19F | E8, A19-46, B8, A19-46 | 365, 367 |
| MX1067 | FIG. 19G | A19-46, B8, A19-46, E8 | 369, 371 |
| MX1068 | FIG. 19H | E8, B8, A19-46, A19-46 | 373, 375 |
| MX1055 | FIG. 19I | E8, B8, E8, A19-46 | 377, 379 |
| MX1056 | FIG. 19J | E8, E8, B8, A19-46 | 381, 383 |
| MX1057 | FIG. 19K | B8, A19-46, E8, E8 | 385, 387 |
| MX1058 | FIG. 19L | E8, A19-46, E8, B8 | 389, 391 |
| MX1041 | FIG. 20A | A19-46, A19-46, E12, E12 | 315, 315 |
| MX1042 | FIG. 20B | E8, E8, E12, E12 | 256, 256 |
| MX1043 | FIG. 20C | E12, E12, A19-46, A19-46 | 317, 317 |
| MX1044 | FIG. 20D | E12, E12, E8, E8 | 319, 319 |
| MX1045 | FIG. 20E | A19-46, E12, A19-46, E12 | 321, 323 |
| MX1046 | FIG. 20F | E8, E12, E8, E12 | 325, 327 |
| MX1069 | FIG. 16A | E12, A19-46, E12, E8 | 257, 258 |
| MX1070 | FIG. 16B | E8, A19-46, E12, E12 | 395, 397 |
| MX1071 | FIG. 16C | E12, E12, A19-46, E8 | 399, 401 |
| MX1072 | FIG. 16D | E8, E12, A19-46, E12 | 403, 405 |
| MX1073 | FIG. 16E | A19-46, A19-46, E12, E8 | 407, 409 |
| MX1074 | FIG. 16F | E8, A19-46, E12, A19-46 | 411, 413 |
| MX1075 | FIG. 16G | A19-46, E12, A19-46, E8 | 415, 417 |
| MX1076 | FIG. 16H | E8, E12, A19-46, A19-46 | 419, 421 |
| MX1077 | FIG. 16I | E8, E12, E8, A19-46 | 423, 425 |
| MX1078 | FIG. 16J | E8, E8, E12, A19-46 | 427, 429 |
| MX1079 | FIG. 16K | E12, A19-46, E8, E8 | 431, 433 |
| MX1080 | FIG. 16L | E8, A19-46, E8, E12 | 435, 437 |
| MX1086 | FIG. 17A | B8, A19-46, E12, E8 | 487, 489 |
| MX1087 | FIG. 17B | E12, A19-46, B8, E8 | 495, 497 |
| MX1088 | FIG. 17C | E8, A19-46, A23-58H/L93VGLTG, B8 | 491, 493 |
| MX1089 | FIG. 17D | E8, A19-46, B8, A23-58H/L93VGLTG | 499, 501 |
| MX1206 | FIG. 17E | E8, A19-46, E12, A23-58H/L93VGLTG | 503, 505 |
| MX1152 | FIG. 18A | A19-46, A19-46, A23-58H/L93VGLTG, | 441, 441 |
| | | E8 | |
| MX1081 | FIG. 18B | E8, A19-46, A23-58H/L93VGLTG, | 439, 441 |
| | | A19-46 | |
| MX1153 | FIG. 18C | A23-58H/L93VGLTG, A23- | 447, 449 |
| | | 58H/L93VGLTG, A19-46, E8 | |
| MX1154 | FIG. 18D | E8, A23-58H/L93VGLTG, A19-46, | 451, 453 |
| | | A23-58H/L93VGLTG | |
| MX1155 | FIG. 18E | A19-46, A19-46, A23-58H/L93VGLTG, | 455, 457 |
| | | E8 | |
| MX1082 | FIG. 18F | E8, A19-46, A23-58H/L93VGLTG, | 459, 461 |
| | | A19-46 | |
| MX1156 | FIG. 18G | A19-46, A23-58H/L93VGLTG, E8, | 463, 465 |
| | | A19-46 | |
| MX1157 | FIG. 18H | E8, A23-58H/L93VGLTG, A19-46, | 467, 469 |
| | | A19-46 | |
| MX1083 | FIG. 18I | E8, A23-58H/L93VGLTG, E8, A19-46 | 471, 473 |
| MX1084 | FIG. 18J | E8, E8, A23-58H/L93VGLTG, A19-46 | 475, 477 |
| MX1158 | FIG. 18K | A23-58H/L93VGLTG, A19-46, E8, E8 | 479, 481 |
| MX1085 | FIG. 18L | E8, A19-46, E8, A23-58H/L93VGLTG | 483, 485 |

### Methods of Use

In some aspects, provided herein are certain methods of use of an antigen binding polypeptide, antigen binding polypeptide complex (e.g., an antibody or antigen binding fragment thereof), polypeptide, polynucleotide, vector, host cell, or pharmaceutical composition provided herein, or a combination thereof. Any of the antigen binding polypeptides, antigen binding polypeptide complexes (e.g., an antibody or antigen binding fragment thereof), polypeptides, polynucleotides, vectors, host cells, or pharmaceutical compositions provided herein described herein may be used in any of the methods and uses of the invention.

In some aspects, provided herein is a method of preventing or treating a SARS-CoV-2 infection, comprising administering an antigen binding polypeptide, antigen binding polypeptide complex (e.g., an antibody or antigen binding fragment thereof), polypeptide, polynucleotide, vector, host cell or pharmaceutical composition provided herein, or a combination thereof. In some aspects, provided herein is an antigen binding polypeptide, antigen binding polypeptide complex (e.g., antibody or antigen binding fragment thereof), polypeptide, polynucleotide, vector, cell, or pharmaceutical composition described herein, or a combination thereof, for use in treating or preventing a SARS-CoV-2 infection. In some aspects, provided herein is the use of an antigen binding polypeptide, antigen binding polypeptide complex (e.g., antibody or antigen binding fragment thereof), polypeptide, polynucleotide, vector, cell, or pharmaceutical composition described herein, or a combination thereof in the manufacture of a medicament for the treatment or prevention of a SARS-CoV-2 infection. In some aspects, preventing or treating a SARS-CoV-2 infection comprises administering a pharmaceutical composition comprising (1) one or more polynucleotides encoding an antigen binding polypeptide or antigen binding polypepetide complex provided herein, and (2) a lipid nanoparticle composition. In some aspects, the lipid nanoparticle composition comprises one or more of (i) a cationic and/or ionizable lipid, (ii) a structural lipid, (iii) a PEGylated lipid, and (iv) a phospholipid, as provided herein.

In some aspects, provided herein is a method of preventing or treating a SARS-CoV-2 infection in a subject in need thereof, comprising administering to the subject a therapeutically effective amount of an antigen binding polypeptide, antigen binding polypeptide complex (e.g., an antibody or antigen binding fragment thereof), polypeptide, polynucleotide, vector, host cell or pharmaceutical composition provided herein, or a combination thereof. In some aspects, provided herein is an antigen binding polypeptide, antigen binding polypeptide complex (e.g., antibody or antigen binding fragment thereof), polypeptide, polynucleotide, vector, cell, or pharmaceutical composition described herein, or a combination thereof, for use in treating or preventing a SARS-CoV-2 infection in a subject in need thereof. In some aspects, provided herein is the use of an antigen binding polypeptide, antigen binding polypeptide complex (e.g., antibody or antigen binding fragment thereof), polypeptide, polynucleotide, vector, cell, or pharmaceutical composition described herein, or a combination thereof in the manufacture of a medicament for the treatment or prevention of a SARS-CoV-2 infection in a subject in need thereof. In some aspects, preventing or treating a SARS-CoV-2 infection in a subject in need thereof comprises administering a pharmaceutical composition comprising (1) one or more polynucleotides encoding an antigen binding polypeptide or antigen binding polypepetide complex provided herein, and (2) a lipid nanoparticle composition. In some aspects, the lipid nanoparticle composition comprises one or more of (i) a cationic and/or ionizable lipid, (ii) a structural lipid, (iii) a PEGylated lipid, and (iv) a phospholipid, as provided herein.

In some aspects, provided herein is a method of preventing or treating coronavirus disease 2019 (COVID-19), comprising administering an antigen binding polypeptide, antigen binding polypeptide complex (e.g., an antibody or antigen binding fragment thereof), polypeptide, polynucleotide, vector, host cell or pharmaceutical composition, or a combination thereof. In some aspects, provided herein is an antigen binding polypeptide, antigen binding polypeptide complex (e.g., antibody or antigen binding fragment thereof), polypeptide, polynucleotide, vector, cell, or pharmaceutical composition described herein, or a combination thereof, for use in treating or preventing coronavirus disease 2019 (COVID-19). In some aspects, provided herein is the use of an antigen binding polypeptide, antigen binding polypeptide complex (e.g., antibody or antigen binding fragment thereof), polypeptide, polynucleotide, vector, cell, or pharmaceutical composition described herein, or a combination thereof in the manufacture of a medicament for the treatment or prevention of coronavirus disease 2019 (COVID-19). In some aspects, preventing or treating coronavirus disease 2019 (COVID-19) comprises administering a pharmaceutical composition comprising (1) one or more polynucleotides encoding an antigen binding polypeptide or antigen binding polypepetide complex provided herein, and (2) a lipid nanoparticle composition. In some aspects, the lipid nanoparticle composition comprises one or more of (i) a cationic and/or ionizable lipid, (ii) a structural lipid, (iii) a PEGylated lipid, and (iv) a phospholipid, as provided herein.

In some aspects, provided herein is a method of preventing or treating coronavirus disease 2019 (COVID-19) in a subject in need thereof, comprising administering to the subject a therapeutically effective amount of an antigen binding polypeptide, antigen binding polypeptide complex (e.g., an antibody or antigen binding fragment thereof), polypeptide, polynucleotide, vector, host cell or pharmaceutical composition, or a combination thereof. In some aspects, provided herein is an antigen binding polypeptide, antigen binding polypeptide complex (e.g., antibody or antigen binding fragment thereof), polypeptide, polynucleotide, vector, cell, or pharmaceutical composition described herein, or a combination thereof, for use in treating or preventing COVID-19 in a subject in need thereof. In some aspects, provided herein is the use of an antigen binding polypeptide, antigen binding polypeptide complex (e.g., antibody or antigen binding fragment thereof), polypeptide, polynucleotide, vector, cell, or pharmaceutical composition described herein, or a combination thereof in the manufacture of a medicament for the treatment or prevention of COVID-19 in a subject in need thereof. In some aspects, preventing or treating COVID-19 comprises administering a pharmaceutical composition comprising (1) one or more polynucleotides encoding an antigen binding polypeptide or antigen binding polypepetide complex provided herein, and (2) a lipid nanoparticle composition. In some aspects, the lipid nanoparticle composition comprises one or more of (i) a cationic and/or ionizable lipid, (ii) a structural lipid, (iii) a PEGylated lipid, and (iv) a phospholipid, as provided herein.

As used herein, the terms “prevent” or “preventing” refer to the prevention of the onset, recurrence or spread, in whole or in part, of a disease or condition provided herein, or a symptom thereof.

As used herein, the terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, “administering” is meant a method of giving a dosage of an antigen binding polypeptide or polypeptide complex (e.g., antibody or antigen binding fragment thereof) to a subject in need thereof (e.g., a patient). Administering can be by any suitable means, including parenteral, intrapulmonary or intranasal. Parenteral infusions include, for example, intramuscular, intravenous, intraarterial, intraperitoneal or subcutaneous administration. Dosing can be by any suitable route, e.g., by injection, such as intravenous or subcutaneous injection. Various dosing schedules including, but not limited to, single or multiple administrations over various time-points, bolus administration, and pulse infusion are contemplated herein.

As used herein, a “therapeutically effective amount” is an amount of an antigen binding polypeptide or antigen binding polypeptide complex (e.g., an antibody or antigen binding fragment thereof) that is sufficient to achieve the desired effect and can vary according to the nature and severity of the condition, and the potency of the polypeptide or polypeptide complex. In some aspects, the therapeutically effective amount is from about 5 ug/mL to about 100 ug/mL, or any value or range of values therein (in some aspects, e.g., over about 30 days post-infusion). In some aspects, the therapeutically effective amount is from about 5 ug/mL to about 75 ug/mL, from about 5 ug/mL to about 50 ug/mL, from about 5 ug/mL to about 25 ug/mL, from about 5 ug/mL to about 10 ug/mL, from about 10 ug/mL to about 100 ug/mL, from about 10 ug/mL to about 75 ug/mL, from about 10 ug/mL to about 50 ug/mL, from about 10 ug/mL to about 25 ug/mL, from about 25 ug/mL to about 100 ug/mL, from about 25 ug/mL to about 75 ug/mL, from about 25 ug/mL to about 50 ug/mL, from about 50 ug/mL to about 100 ug/mL, from about 50 ug/mL to about 75 ug/mL, or from about 75 ug/mL to about 100 ug/mL. In some aspects, the therapeutically effective amount is about 5 ug/mL, about 10 ug/mL, about 25 ug/mL, about 50 ug/mL, about 75 ug/mL, or about 100 ug/mL.

In some aspects, an antigen binding polypeptide or polypeptide complex can be delivered by administering a polynucleotide, vector, or host cell that encodes the antigen binding polypeptide or polypeptide complex. In some aspects, an antigen binding polypeptide or polypeptide complex thereof can be delivered by administering a pharmaceutical composition containing the polypeptide or polypeptide complex. A therapeutic effect is the relief, to at least some extent, of one or more symptoms of the disease or disorder, and can include curing a disease or disorder. “Curing” means that the symptoms of active disease are eliminated. However, certain long-term or permanent effects of a disease or disorder can exist even after a cure is obtained.

As used herein, the term “subject” means a human or a non-human mammal, e.g., a dog, cat, mouse, rat, cow, sheep, pig, goat, non-human primate or bird, e.g., chicken, as well as any other vertebrate or invertebrate. In some aspects, the subject is a human. In some aspects, the subject is a veterinary animal. In some aspects, the subject is a mammal.

In some aspects, provided herein is a method of diagnosing a subject as being infected with a SARS-CoV-2 virus or suspected of being infected with a SARS-CoV-2 virus, comprising (i) contacting a sample obtained from the subject with the antigen binding polypeptide or polypeptide complex (e.g., an antibody or antigen binding fragment thereof) provided herein; (ii) detecting the presence or absence of a virus complex which contains the polypeptide or polypeptide complex and a SARS-CoV-2 virus, virion or fragment thereof; and (iii) diagnosing the subject as being infected with a SARS-CoV-2 virus or suspected of being infected with a SARS-CoV-2 virus when the presence of the virus complex is detected. In some aspects, the method is performed in vitro or ex vivo.

In some aspects, provided herein is a method of diagnosing a subject as not being infected with a SARS-CoV-2 virus or not suspected of being infected with a SARS-CoV-2 virus, comprising (i) contacting a sample obtained from the subject with the antigen binding polypeptide or polypeptide complex (e.g., an antibody or antigen binding fragment thereof) provided herein; (ii) detecting the presence or absence of a virus complex which contains the polypeptide or polypeptide complex and a SARS-CoV-2 virus, virion or fragment thereof, and (iii) diagnosing the subject as not being infected with a SARS-CoV-2 virus or not being suspected of being infected with a SARS-CoV-2 virus when the presence of the virus complex is not detected. In some aspects, the method is performed in vitro or ex vivo.

In some aspects, provided herein is a method of diagnosing a subject as having COVID-19 or suspected of having COVID-19, comprising (i) contacting a sample obtained from the subject with an antigen binding polypeptide or polypeptide complex (e.g., an antibody or antigen binding fragment thereof) provided herein; (ii) detecting the presence or absence of a virus complex which contains the polypeptide or polypeptide complex and a SARS-CoV-2 virus, virion or fragment thereof, and (iii) diagnosing the subject as having COVID-19 or suspected or having COVID-19 when the presence of the virus complex is detected. In some aspects, the method is performed in vitro or ex vivo.

In some aspects, provided herein is a method of diagnosing a subject as not having COVID-19 or not suspected of having COVID-19, comprising (i) contacting a sample obtained from the subject with an antigen binding polypeptide or polypeptide complex (e.g., an antibody or antigen binding fragment thereof) provided herein; (ii) detecting the presence or absence of a virus complex which contains the polypeptide or polypeptide complex and a SARS-CoV-2 virus, virion or fragment thereof, and (iii) diagnosing the subject as not having COVID-19 or not suspected of having COVID-19 when the presence of the virus complex is not detected. In some aspects, the method is performed in vitro or ex vivo.

The term “sample,” as used herein, refers to a composition that is obtained or derived from a subject that contains a cellular and/or other molecular entity that is to be characterized and/or identified, for example, based on physical, biochemical, chemical, and/or physiological characteristics. A sample includes, but is not limited to, nasal fluid or discharge (e.g., from a nasal swab), tissue, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, tissue culture medium, tissue extracts such as homogenized tissue, cellular extracts, and combinations thereof.

Methods for collecting, processing and storing a sample are known, and described, for example, in Vaught et al., IARC Sci. Pub., Unit 2. Chapter 3, pp. 23-42, 2011; CDC Interim Guidelines for Collecting and Handling of Clinical Specimens for COVID-19 Testing, Oct. 25, 2021; and Minghetti et al., Recommendations for collection, transport and storage of COVID-19 biological samples, ISS working group on Translational Research COVID-19, Istituto Superiore di Sanita, Apr. 15, 2020.

Methods for detecting the binding of an antigen binding polypeptide or polypeptide complex (e.g., antibody or antigen binding fragment thereof) provided herein and a SARS-CoV-2 virus, virion or fragment thereof are also known. Such methods include, but are not limited to, immunosorbent assay (ELISA), immunospot assay, lateral flow assay, flow cytometry, immunohistochemistry or western blot. See, e.g., Kumar et al., VirusDisease, 31:97-105, 2020; Gong et al., Front. Mol. Biosci. Jul. 23, 2021; and Espejo et al., Am. J. Clin. Pathol., 154(3)293-304, 2020.

In some aspects, the disclosure provides a method of delivering an antigen binding polypeptide or antigen binding polypeptide complex provided herein. In some aspects, the method includes administering to a subject a pharmaceutical composition provided herein. In some aspects, the pharmaceutical composition comprises (1) one or more polynucleotides encoding an antigen binding polypeptide or antigen binding polypeptide complex provided herein, and (2) a lipid nanoparticle composition. In some aspects, the lipid nanoparticle composition comprises one or more of (i) a cationic and/or ionizable lipid, (ii) a structural lipid, (iii) a PEGylated lipid, and (iv) a phospholipid, as provided herein.

In some aspects, the disclosure provides a method of producing an antigen binding polypeptide or antigen binding polypeptide complex provided herein in a cell. In some aspects, the method includes administering to a subject a pharmaceutical composition provided herein. In some aspects, the pharmaceutical composition comprises (1) one or more polynucleotides encoding an antigen binding polypeptide or antigen binding polypeptide complex provided herein, and (2) a lipid nanoparticle composition. In some aspects, the lipid nanoparticle composition comprises one or more of (i) a cationic and/or ionizable lipid, (ii) a structural lipid, (iii) a PEGylated lipid, and (iv) a phospholipid, as provided herein.

In some aspects, the disclosure provides a method of treating a disease or disorder provided herein. In some aspects, the method includes administering to a subject a pharmaceutical composition provided herein. In some aspects, the pharmaceutical composition comprises (1) one or more polynucleotides encoding an antigen binding polypeptide or antigen binding polypeptide complex provided herein, and (2) a lipid nanoparticle composition. In some aspects, the lipid nanoparticle composition comprises one or more of (i) a cationic and/or ionizable lipid, (ii) a structural lipid, (iii) a PEGylated lipid, and (iv) a phospholipid, as provided herein.

In some aspects, the antigen binding polypeptide or polypeptide complex (e.g., an antibody or antigen binding fragment thereof) has greater neutralization potency against SARS-CoV-2 virus than a monospecific polypeptide, polypeptide complex, antibody or fragment that specifically binds to one of the same antigens.

In some aspects, the antigen binding polypeptide or polypeptide complex (e.g., an antibody or antigen binding fragment thereof) is bispecific, trispecific or tetraspecific and has greater neutralization potency against SARS-CoV-2 virus than a monospecific polypeptide, polypeptide complex, antibody or fragment that specifically binds to one of the same antigens the bispecific, trispecific or tetraspecific polypeptide or polypeptide complex.

In some aspects, the antigen binding polypeptide or polypeptide complex (e.g., an antibody or antigen binding fragment thereof) has greater neutralization potency against SARS-CoV-2 virus than a mixture of monospecific polypeptides, polypeptide complexes, antibodies or fragments that specifically bind to the same antigens.

In some aspects, the antigen binding polypeptide or polypeptide complex (e.g., an antibody or antigen binding fragment thereof) is bispecific, trispecific or tetraspecific and has greater neutralization potency against SARS-CoV-2 virus than a mixture of monospecific polypeptides, polypeptide complexes, antibodies or fragments that specifically bind to the same antigens as the bispecific, trispecific or tetraspecific polypeptide or polypeptide complex.

In some aspects, the antigen binding polypeptide complex (e.g., an antibody or antigen binding fragment thereof), polypeptide, polynucleotide, vector, host cell, or pharmaceutical composition provided herein neutralizes multiple SARS-CoV-2 variants. In some aspects, the antigen binding polypeptide complex (e.g., an antibody or antigen binding fragment thereof), polypeptide, polynucleotide, vector, host cell, or pharmaceutical composition provided herein neutralizes diverse SARS-CoV-2 variants. In some aspects, the antigen binding polypeptide complex (e.g., an antibody or antigen binding fragment thereof), polypeptide, polynucleotide, vector, host cell, or pharmaceutical composition provided herein provides broad coverage against SARS-CoV-2 variants.

In some aspects, the antigen binding polypeptide complex (e.g., an antibody or antigen binding fragment thereof), polypeptide, polynucleotide, vector, host cell, or pharmaceutical composition provided herein reduces viral load to no more than from about 1×10⁶ per gram of tissue (TCID50/g) to about 2×10⁶ per gram of tissue. In some aspects, the antigen binding polypeptide complex (e.g., an antibody or antigen binding fragment thereof), polypeptide, polynucleotide, vector, host cell, or pharmaceutical composition provided herein reduces viral load to no more than from about 1×10⁶ per gram of tissue to about 1.5×10⁶ per gram of tissue. In some aspects, the antigen binding polypeptide complex (e.g., an antibody or antigen binding fragment thereof), polypeptide, polynucleotide, vector, host cell, or pharmaceutical composition provided herein reduces viral load to no more than about 1.4×10⁶ per gram of tissue.

In some aspects, the SARS-CoV-2 of the SARS-CoV-2 protein to which an antigen binding polypeptide or polypeptide complex provided herein specifically binds is a variant of concern (VOC). In some aspects, the SARS-CoV-2 of the SARS-CoV-2 protein to which an antigen binding polypeptide or polypeptide complex provided herein specifically binds is a variant under monitoring (e.g., variant with genetic changes suspected to affect virus characteristics and some indication of posing a future risk, but further studies are needed).

In some aspects, the SARS-CoV-2 of the SARS-CoV-2 protein to which an antigen binding polypeptide or polypeptide complex provided herein specifically binds is a Lambda (C.37), Mu (B.1.621), Epsilon (B.1.429, B.1.427, CAL.20C), Zeta (P.2), Eta (B.1.525), Theta (P.3), Iota (B.1.526), or Kappa (B.1.617.1) variant, or a combination thereof. In some aspects, the SARS-CoV-2 of the SARS-CoV-2 protein to which an antigen binding polypeptide or polypeptide complex provided herein specifically binds is a AZ.5, C.1.2, B.1.617.1, B.1.526, B.1.525, B.1.630, B.1.640, AV.1, AT.1, R.1, B.1.466.2, B.1.1.519, C.36.3, B.1.214.2, B.1.427, B.1.429, B.1.1.523, B.1.619, B.1.620, B.1.1.207, B.1.1.317, B.1.616, B.1.618, or B.1.640.2 variant, or a combination thereof.

In some aspects, the SARS-CoV-2 of the SARS-CoV-2 protein to which an antigen binding polypeptide or polypeptide complex provided herein specifically binds is an Alpha (B.1.1.7), Beta (B.1.351), Gamma (P.1), Delta (B.1.617.2), or Omicron (B.1.1.529) variant, or a combination thereof. In some aspects, the Omicron variant is BA.1, BA.2, BA.2.12.1, BA, 4, BA.4/5, BA.5, BQ.1, BQ.1.1, BA.2.75, BA.2.75.2, BA.4.6, BQ.1.1, BJ.1, XBB, XBB1.5, BF.7, CH.1.1, or a combination thereof.

In some aspects, the SARS-CoV-2 protein has an amino acid mutation of one or more of the following: F2L, L5F, L51, V6F, L7V, P9L, S12C, S13I, Q14H, C15F, N17K, L18F, T20I, T22N, T22A, T22I, Q23K, P25S, A27S, A27V, T29I, F32L, R34C, H49Y, S50L, T51I, Q52L, Q52H, L54F, L54W, F55I, P57L, H69Y, S71F, G72V, T73I, G75V, T76I, F79L, D80N, D80Y, N87Y, D88N, D88E, D88Y, D88A, V90F, T95A, T95I, E96D, K97T, S98F, R1021, 1105L, D111N, K113R, L118F, V130A, E132D, C136R, D138H, L141-, L141F, G142V, G142-, V143F, V143-, Y144-, Y144V, Y145H, H146Y, K147E, N148S, S151I, W152C, M153T, M153V, M153I, E154V, F157L, R158S, L176F, M177I, D178N, G181V, L189F, R190K, I203M, I210-, R214L, D215Y, D215G, L216F, Q218L, F220L, S221L, A222V, A222P, D228H, L229F, Q239R, T240I, L242F, A243S, A243V, H245R, H245Y, R246K, D253Y, D253G, S254F, S256L, W258L, G261V, G261R, A262S, Y265C, V267L, R273S, E281Q, A288S, L293V, D294E, P295S, E298G, T307I, V308L, E309Q, Q314K, Q314L, Q314H, T315I, Q321L, T323I, P330S, A344S, T345S, A348T, A348S, N354K, R357K, V367F, V382L, P384L, V395I, R403K, V407I, A411S, G413R, K417T, N439K, N440K, L441I, G446V, R457K, K458Q, G476S, S477N, P479L, V483A, E484Q, E484K, Q493L, S494P, L452R, Y453F, Y508H, N501Y, H519Q, A520S, A522V, K529E, G545S, T547I, L552F, T553I, E554D, K558N, A570V, T572I, D574Y, E583D, I584V, S5961, 1598V, N603H, Q613H, D614G, V615F, T618A, P621S, V622F, V622I, V622A, A623S, H625Y, A626V, P631S, W633R, G639V, S640F, A647S, A647V, E654Z, E654K, H655Y, N658Y, A668S, A672V, Q675R, Q675H, T676S, T676I, Q677H, Q677R, T678I, P681L, P681R, P681H, R682W, A684S, A684T, V687L, A688V, A688S, S689I, S691F, S698L, N703D, S704L, V705F, A706V, I714M, T716I, I720V, T724A, M7311, T732A, T732I, G744V, D745G, N751D, L754F, R765S, R765H, T768I, G769A, A771S, T778I, Q779H, E780Q, A783S, D808V, D808G, P809S, I818V, L822F, D830H, D830Y, Q836P, Q836L, G838D, A845S, A845D, A845V, A845D, A845S, A846V, R847I, K854R, N856S, T859I, D867N, A879V, A879S, F888L, A893E, A893V, E918V, L922F, A924S, A924V, S929I, D936H, D936Y, L938F, S939F, T941I, G946V, A958S, N969S, L981F, T1006I, V1008T, T1009I, A1016S, A1020V, F1052L, P1053T, L1063F, V1065L, A1070V, Q1071H, E1072V, K1073N, A1078V, A1078S, G1085R, K1086N, R1091L, H1101Y, V1104L, P1112L, D1118Y, Ti 1201, V1122L, S1123P, G1124V, G1124C, V1129A, 11130M, T1136I, D1139H, L1141F, D1146H, S1147L, D1153Y, P1162Q, P1162S, P1162Q, P1162S, D1163Y, G1167V, D1168H, V1176F, N1187Y, K1191N, N1192T, E1195Q, L1203F, K1205N, E1207A, 1219 V, G1219S, I1221T, V1228L, M1229I, V1230L, T1231I, T1231A, C1235F, M1237I, M1237V, M1237T, T1238I, K1245N, C1247F, G1251R, D1259H, S1261F, P1263L, V1264L, a deletion of residues 69 and 70, a deletion of residues 246-252 and D253N, or a combination thereof.

In some aspects, provided herein is a method of preventing immune escape of a SARS-CoV2 virus in a subject infected with the SARS-CoV2 virus, comprising administering to the subject an effective amount of an antigen binding polypeptide or polypeptide complex provided herein, an antibody or antigen binding fragment thereof provided herein, a pharmaceutical composition provided herein, or a combination thereof. In some aspects, provided herein is an antigen binding polypeptide, antigen binding polypeptide complex (e.g., antibody or antigen binding fragment thereof), polypeptide, polynucleotide, vector, cell, or pharmaceutical composition described herein, or a combination thereof, for use in preventing immune escape of a SARS-CoV-2 variant in a subject in need theref. In some aspects, provided herein is the use of an antigen binding polypeptide, antigen binding polypeptide complex (e.g., antibody or antigen binding fragment thereof), polypeptide, polynucleotide, vector, cell, or pharmaceutical composition described herein, or a combination thereof in the manufacture of a medicament for preventing immune escape of a SARS-CoV-2 variant in a subject in need thereof. In some aspects, provided herein is an antigen binding polypeptide, antigen binding polypeptide complex (e.g., antibody or antigen binding fragment thereof), polypeptide, polynucleotide, vector, cell, or pharmaceutical composition described herein, or a combination thereof, for use in treating or preventing infection with a SARS-CoV-2 variant in a subject in need thereof, wherein the antigen binding polypeptide complex (e.g., antibody or antigen binding fragment thereof), polypeptide, polynucleotide, vector, cell, pharmaceutical composition or combination thereof prevents immune escape of the SARS-CoV-2 variant in the subject. In some aspects, provided herein is the use of an antigen binding polypeptide, antigen binding polypeptide complex (e.g., antibody or antigen binding fragment thereof), polypeptide, polynucleotide, vector, cell, or pharmaceutical composition described herein, or a combination thereof in the manufacture of a medicament for treating or preventing infection with a SARS-CoV-2 variant in a subject in need thereof, wherein the antigen binding polypeptide complex (e.g., antibody or antigen binding fragment thereof), polypeptide, polynucleotide, vector, cell, pharmaceutical composition or combination thereof prevents immune escape of the SARS-CoV-2 variant in the subject. In some aspects, preventing immune escape of a SARS-CoV-2 variant comprises administering a pharmaceutical composition comprising (1) one or more polynucleotides encoding an antigen binding polypeptide or antigen binding polypepetide complex provided herein, and (2) a lipid nanoparticle composition. In some aspects, the lipid nanoparticle composition comprises one or more of (i) a cationic and/or ionizable lipid, (ii) a structural lipid, (iii) a PEGylated lipid, and (iv) a phospholipid, as provided herein.

In some aspects, provided herein is a method of preventing immune escape of a SARS-CoV2 virus in a subject infected with the SARS-CoV2 virus, comprising administering to the subject an effective amount of an anti-SARS-CoV2 multivalent antibody that exhibits improved prevention of immune escape compared to (i) a monovalent anti-SARS-CoV2 antibody that binds to one of the same antigens as the anti-SARS-CoV2 multivalent antibody, and/or (ii) a combination of monovalent anti-SARS-CoV2 antibodies that binds to the same antigens as the anti-SARS-CoV2 multivalent antibody. In some aspects, provided herein is an anti-SARS-CoV2 multivalent antibody for use in a method of preventing immune escape of a SARS-CoV2 virus in a subject infected with the SARS-CoV2 virus, wherein the anti-SARS-CoV2 multivalent antibody exhibits improved prevention of immune escape compared to (i) a monovalent anti-SARS-CoV2 antibody that binds to one of the same antigens as the anti-SARS-CoV2 multivalent antibody, and/or (ii) a combination of monovalent anti-SARS-CoV2 antibodies that binds to the same antigens as the anti-SARS-CoV2 multivalent antibody. In some aspects, provided herein is the use of an anti-SARS-CoV2 multivalent antibody in the manufacture of a medicament for preventing immune escape of a SARS-CoV2 virus in a subject infected with the SARS-CoV2 virus, wherein the anti-SARS-CoV2 multivalent antibody exhibits improved prevention of immune escape compared to (i) a monovalent anti-SARS-CoV2 antibody that binds to one of the same antigens as the anti-SARS-CoV2 multivalent antibody, and/or (ii) a combination of monovalent anti-SARS-CoV2 antibodies that binds to the same antigens as the anti-SARS-CoV2 multivalent antibody. In some aspects, provided herein is an anti-SARS-CoV2 multivalent antibody for use in preventing or treating a SARS-CoV-2 virus infection in a subject in need thereof, wherein the anti-SARS-CoV2 multivalent antibody prevents immune escape of the SARS-CoV-2 virus in the subject, and wherein the anti-SARS-CoV2 multivalent antibody exhibits improved prevention of immune escape compared to (i) a monovalent anti-SARS-CoV2 antibody that binds to one of the same antigens as the anti-SARS-CoV2 multivalent antibody, and/or (ii) a combination of monovalent anti-SARS-CoV2 antibodies that binds to the same antigens as the anti-SARS-CoV2 multivalent antibody. In some aspects, provided herein is the use of an anti-SARS-CoV2 multivalent antibody in the manufacture of a medicament for preventing or treating a SARS-CoV-2 virus infection in a subject in need thereof, wherein the anti-SARS-CoV2 multivalent antibody prevents immune escape of the SARS-CoV-2 virus in the subject, and wherein the anti-SARS-CoV2 multivalent antibody exhibits improved prevention of immune escape compared to (i) a monovalent anti-SARS-CoV2 antibody that binds to one of the same antigens as the anti-SARS-CoV2 multivalent antibody, and/or (ii) a combination of monovalent anti-SARS-CoV2 antibodies that binds to the same antigens as the anti-SARS-CoV2 multivalent antibody.

In some aspects, provided herein is a method of neutralizing a SARS-CoV-2 variant, comprising administering an antigen binding polypeptide complex (e.g., an antibody or antigen binding fragment thereof), polypeptide, polynucleotide, vector, host cell or pharmaceutical composition provided herein, or a combination thereof. In some aspects, provided herein is a method of neutralizing a SARS-CoV-2 variant in a subject in need thereof, comprising administering to the subject an effective amount of an antigen binding polypeptide complex (e.g., an antibody or antigen binding fragment thereof), polypeptide, polynucleotide, vector, host cell or pharmaceutical composition provided herein, or a combination thereof. In some aspects, multiple SARS-CoV-2 variants are neutralized. In some aspects, diverse SARS-CoV-2 variants are neutralized. In some aspects, provided herein is an antigen binding polypeptide complex (e.g., antibody or antigen binding fragment thereof), polypeptide, polynucleotide, vector, cell, or pharmaceutical composition described herein, or a combination thereof, for use in a method of neutralizing a SARS-CoV-2 variant in a subject in need thereof. In some aspects, provided herein is the use of an antigen binding polypeptide complex (e.g., antibody or antigen binding fragment thereof), polypeptide, polynucleotide, vector, cell, or pharmaceutical composition described herein, or a combination thereof in the manufacture of a medicament for neutralizing a SARS-CoV-2 variant in a subject in need thereof. In some aspects, provided herein is an antigen binding polypeptide complex (e.g., antibody or antigen binding fragment thereof), polypeptide, polynucleotide, vector, cell, or pharmaceutical composition described herein, or a combination thereof, for use in preventing or treating infection with a SARS-CoV-2 variant in a subject in need thereof, wherein the antigen binding polypeptide complex (e.g., antibody or antigen binding fragment thereof), polypeptide, polynucleotide, vector, cell, pharmaceutical composition or combination thereof neutralizes the SARS-CoV-2 variant in the subject. In some aspects, provided herein is the use of an antigen binding polypeptide complex (e.g., antibody or antigen binding fragment thereof), polypeptide, polynucleotide, vector, cell, or pharmaceutical composition described herein, or a combination thereof in the manufacture of a medicament for treating or preventing infection with a SARS-CoV-2 variant in a subject in need thereof, wherein the antigen binding polypeptide complex (e.g., antibody or antigen binding fragment thereof), polypeptide, polynucleotide, vector, cell, pharmaceutical composition or combination thereof neutralizes the SARS-CoV-2 variant in the subject. In some aspects, treating or preventing a SARS-CoV-2 infection by neutralizing a SARS-CoV-2 variant comprises administering a pharmaceutical composition comprising (1) one or more polynucleotides encoding an antigen binding polypeptide or antigen binding polypepetide complex provided herein, and (2) a lipid nanoparticle composition. In some aspects, the lipid nanoparticle composition comprises one or more of (i) a cationic and/or ionizable lipid, (ii) a structural lipid, (iii) a PEGylated lipid, and (iv) a phospholipid, as provided herein.

In some aspects, provided herein is a method of increasing neutralization potency against a SARS-CoV-2 virus in a subject infected with the SARS-CoV2 virus, comprising administering to the subject an effective amount of an antigen binding polypeptide or polypeptide complex provided herein, an antibody or antigen binding fragment thereof provided herein, or a combination thereof. In some aspects, provided herein is an antigen binding polypeptide, antigen binding polypeptide complex (e.g., antibody or antigen binding fragment thereof), polypeptide, polynucleotide, vector, cell, or pharmaceutical composition described herein, or a combination thereof, for use in a method of increasing neutralization potency against a SARS-CoV-2 variant in a subject in need thereof. In some aspects, provided herein is the use of an antigen binding polypeptide, antigen binding polypeptide complex (e.g., antibody or antigen binding fragment thereof), polypeptide, polynucleotide, vector, cell, or pharmaceutical composition described herein, or a combination thereof in the manufacture of a medicament for increasing neutralization potency against a SARS-CoV-2 variant in a subject in need thereof. In some aspects, provided herein is an antigen binding polypeptide, antigen binding polypeptide complex (e.g., antibody or antigen binding fragment thereof), polypeptide, polynucleotide, vector, cell, or pharmaceutical composition described herein, or a combination thereof, for use in treating or preventing infection with a SARS-CoV-2 variant in a subject in need thereof, wherein the antigen binding polypeptide complex (e.g., antibody or antigen binding fragment thereof), polypeptide, polynucleotide, vector, cell, pharmaceutical composition or combination thereof increases neutralization potency against the SARS-CoV-2 variant in the subject. In some aspects, provided herein is the use of an antigen binding polypeptide, antigen binding polypeptide complex (e.g., antibody or antigen binding fragment thereof), polypeptide, polynucleotide, vector, cell, or pharmaceutical composition described herein, or a combination thereof in the manufacture of a medicament for treating or preventing infection with a SARS-CoV-2 variant in a subject in need thereof, wherein the antigen binding polypeptide complex (e.g., antibody or antigen binding fragment thereof), polypeptide, polynucleotide, vector, cell, pharmaceutical composition or combination thereof increases neutralization potency against the SARS-CoV-2 variant in the subject. In some aspects, increasing neutralization potency against a SARS-CoV-2 variant comprises administering a pharmaceutical composition comprising (1) one or more polynucleotides encoding an antigen binding polypeptide or antigen binding polypepetide complex provided herein, and (2) a lipid nanoparticle composition. In some aspects, the lipid nanoparticle composition comprises one or more of (i) a cationic and/or ionizable lipid, (ii) a structural lipid, (iii) a PEGylated lipid, and (iv) a phospholipid, as provided herein.

In some aspects, provided herein is a method of increasing neutralization potency against a SARS-CoV-2 virus in a subject infected with the SARS-CoV2 virus, comprising administering to the subject an effective amount of an anti-SARS-CoV2 multivalent antibody that exhibits increased neutralization potency compared to (i) a monovalent anti-SARS-CoV2 antibody that binds to one of the same antigens as the anti-SARS-CoV2 multivalent antibody and/or (ii) a combination of monovalent anti-SARS-CoV2 antibodies that binds to the same antigens as the anti-SARS-CoV2 multivalent antibody. In some aspects, provided herein is an anti-SARS-CoV2 multivalent antibody for use in a method of increasing neutralization potency against a SARS-CoV-2 virus in a subject infected with the SARS-CoV2 virus, wherein the anti-SARS-CoV2 multivalent antibody exhibits increased neutralization potency compared to (i) a monovalent anti-SARS-CoV2 antibody that binds to one of the same antigens as the anti-SARS-CoV2 multivalent antibody and/or (ii) a combination of monovalent anti-SARS-CoV2 antibodies that binds to the same antigens as the anti-SARS-CoV2 multivalent antibody. In some aspects, provided herein is the use of an anti-SARS-CoV2 multivalent antibody in the manufacture of a medicament for increasing neutralization potency against a SARS-CoV-2 virus in a subject infected with the SARS-CoV2 virus, wherein the anti-SARS-CoV2 multivalent antibody exhibits increased neutralization potency compared to (i) a monovalent anti-SARS-CoV2 antibody that binds to one of the same antigens as the anti-SARS-CoV2 multivalent antibody and/or (ii) a combination of monovalent anti-SARS-CoV2 antibodies that binds to the same antigens as the anti-SARS-CoV2 multivalent antibody. In some aspects, provided herein is an anti-SARS-CoV2 multivalent antibody for use in preventing or treating a SARS-CoV-2 virus infection in a subject in need thereof, wherein the anti-SARS-CoV2 multivalent antibody increases neutralization potency against the SARS-CoV-2 virus in the subject, and wherein the anti-SARS-CoV2 multivalent antibody exhibits increased neutralization potency compared to (i) a monovalent anti-SARS-CoV2 antibody that binds to one of the same antigens as the anti-SARS-CoV2 multivalent antibody and/or (ii) a combination of monovalent anti-SARS-CoV2 antibodies that binds to the same antigens as the anti-SARS-CoV2 multivalent antibody. In some aspects, provided herein is the use of an anti-SARS-CoV2 multivalent antibody in the manufacture of a medicament for preventing or treating a SARS-CoV-2 virus infection in a subject in need thereof, wherein the anti-SARS-CoV2 multivalent antibody increases neutralization potency against the SARS-CoV-2 virus in the subject, and wherein the anti-SARS-CoV2 multivalent antibody exhibits increased neutralization potency compared to (i) a monovalent anti-SARS-CoV2 antibody that binds to one of the same antigens as the anti-SARS-CoV2 multivalent antibody and/or (ii) a combination of monovalent anti-SARS-CoV2 antibodies that binds to the same antigens as the anti-SARS-CoV2 multivalent antibody.

In some aspects, the anti-SARS-CoV2 multivalent antibody is administered in a pharmaceutical composition. In some aspects, the pharmaceutical composition further comprises a pharmaceutically acceptable carrier.

In some aspects, the anti-SARS-CoV2 multivalent antibody exhibits improved prevention of immune escape compared to a monovalent anti-SARS-CoV2 antibody that binds to one of the same antigens as the anti-SARS-CoV2 multivalent antibody.

In some aspects, the anti-SARS-CoV2 multivalent antibody exhibits increased neutralization potency compared to a combination of monovalent anti-SARS-CoV2 antibodies that binds to the same antigens as the anti-SARS-CoV2 multivalent antibody.

In some aspects, the anti-SARS-CoV2 multispecific antibody prevents immune escape of the anti-SARS-CoV2 multivalent antibody for at least 2, at least 3, at least 4, at least 5, at least 6, or at least 7 selection rounds as measured by an antibody escape assay. In some aspects, the anti-SARS-CoV2 multispecific antibody prevents immune escape of the anti-SARS-CoV2 multivalent antibody for about 2 to about 7 selection rounds as measured by an antibody escape assay, or any value or range of values therein, for example, for about 3 to about 7 selection rounds, about 4 to about 7 selection rounds, about 5 to about 7 selection rounds, about 6 to about 7 selection rounds, about 2 to about 6 selection rounds, about 3 to about 6 selection rounds, about 4 to about 6 selection rounds, about 5 to about 6 selection rounds, about 2 to about 5 selection rounds, about 3 to about 5 selection rounds, about 4 to about 5 selection rounds, about 2 to about 4 selection rounds, about 3 to about 4 selection rounds, or about 2 to about 3 selection rounds as measured by an antibody escape assay. In some aspects, the anti-SARS-CoV2 multispecific antibody prevents immune escape of the anti-SARS-CoV2 multivalent antibody for about 2, about 3, about 4, about 5, about 6 or about 7 selection rounds as measured by an antibody escape assay.

In some aspects, the anti-SARS-CoV2 multispecific antibody prevents immune escape of the anti-SARS-CoV2 multivalent antibody following at least 2, at least 3, at least 4, at least 5, at least 6, or at least 7 exposures to the subject by the SARS-CoV2 virus. In some aspects, the anti-SARS-CoV2 multispecific antibody prevents immune escape of the anti-SARS-CoV2 multivalent antibody following about 2 to about 7 exposures to the subject by the SARS-CoV2 virus, or any value or range of values therein, for example, for about 3 to about 7 exposures, about 4 to about 7 exposures, about 5 to about 7 exposures, about 6 to about 7 exposures, about 2 to about 6 exposures, about 3 to about 6 exposures, about 4 to about 6 exposures, about 5 to about 6 exposures, about 2 to about 5 exposures, about 3 to about 5 exposures, about 4 to about 5 exposures, about 2 to about 4 exposures, about 3 to about 4 exposures, or about 2 to about 3 exposures to the subject by the SARS-CoV2 virus. In some aspects, the anti-SARS-CoV2 multispecific antibody prevents immune escape of the anti-SARS-CoV2 multivalent antibody following about 2, about 3, about 4, about 5, about 6 or about 7 exposures to the subject by the SARS-CoV2 virus.

In some aspects, the anti-SARS-CoV2 multispecific antibody is at least 100-fold, at least 500-fold, at least 1,000-fold, or at least 2,000-fold more effective in inhibiting growth of the SARS-CoV2 virus compared to (i) a monovalent anti-SARS-CoV2 antibody that binds to one of the same antigens as the anti-SARS-CoV2 multivalent antibody and/or (ii) a combination of monovalent anti-SARS-CoV2 antibodies that binds to the same antigens as the anti-SARS-CoV2 multivalent antibody. In some aspects, the anti-SARS-CoV2 multispecific antibody is from about 100-fold to about 2,000-fold more effective in inhibiting growth of the SARS-CoV2 virus compared to (i) a monovalent anti-SARS-CoV2 antibody that binds to one of the same antigens as the anti-SARS-CoV2 multivalent antibody and/or (ii) a combination of monovalent anti-SARS-CoV2 antibodies that binds to the same antigens as the anti-SARS-CoV2 multivalent antibody, or any value or range of values therein, for example, from about 500-fold to about 2,000-fold, about 1,000-fold to about 2,000-fold, about 100-fold to about 1,000-fold, about 500-fold to about 1,000-fold, or about 100-fold to about 500-fold. In some aspects, the anti-SARS-CoV2 multispecific antibody is about 100-fold, about 500-fold, about 1,000-fold, or about 2,000-fold more effective in inhibiting growth of the SARS-CoV2 virus compared to (i) a monovalent anti-SARS-CoV2 antibody that binds to one of the same antigens as the anti-SARS-CoV2 multivalent antibody and/or (ii) a combination of monovalent anti-SARS-CoV2 antibodies that binds to the same antigens as the anti-SARS-CoV2 multivalent antibody. In some aspects, the anti-SARS-CoV2 multivalent antibody comprises a first antigen binding site that specifically binds to a SARS-CoV-2 protein and a second antigen binding site that specifically binds to a SARS-CoV-2 protein.

In some aspects, the first antigen binding site is different than the second antigen binding site.

In some aspects, the first antigen binding site comprises a first heavy chain variable domain and a first light chain variable domain, and the second antigen binding site comprises a second heavy chain variable domain and a second light chain variable domain.

In some aspects, the first and second heavy chain variable regions each comprise CDR1, CDR2 and CDR3 regions, and the first and second light chain variable region each comprise CDR1, CDR2 and CDR3 regions.

In some aspects, the CDR1 of the first heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664; the CDR2 of the first heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; the CDR3 of the first heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666; the CDR1 of the first light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:39, 47, 55, 63, 71, 167, 175, 183, 260, 268, 276, 284, 517, 525, 533, 541, 549, 557, 565, 573, 581, 589, 597, 605, 613, 621, 643, 651, 660 and 668; the CDR2 of the first light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:40, 48, 56, 64, 72, 168, 176, 184, 261, 269, 277 (having the sequence of LGS), 285, 518 (having the sequence of DAS), 526 (having the sequence of DVS), 534 (having the sequence of EDS), 542 (having the sequence of KDS), 550 (having the sequence of DAS), 558 (having the sequence of AAS), 566 (having the sequence of GAS), 574 (having the sequence of DDS), 582 (having the sequence of KDS), 590 (having the sequence of SAS), 598 (having the sequence of SAS), 606 (having the sequence of GAS), 614 (having the sequence of GAS), 622 (having the sequence of GAS), 644 (having the sequence of GAS), 652 (having the sequence of SAS), 661 (having the sequence of GAS) and 669 (having the sequence of DAS); the CDR3 of the first light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:41, 49, 57, 65, 73, 169, 177, 185, 262, 270, 278, 286, 519, 527, 535, 543, 551, 559, 567, 575, 583, 591, 599, 607, 615, 623, 645, 653, 662 and 670; the CDR1 of the second heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664; the CDR2 of the second heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; the CDR3 of the second heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666; the CDR1 of the second light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:39, 47, 55, 63, 71, 167, 175, 183, 260, 268, 276, 284, 517, 525, 533, 541, 549, 557, 565, 573, 581, 589, 597, 605, 613, 621, 643, 651, 660 and 668; the CDR2 of the second light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:40, 48, 56, 64, 72, 168, 176, 184, 261, 269, 277 (having the sequence of LGS), 285, 518 (having the sequence of DAS), 526 (having the sequence of DVS), 534 (having the sequence of EDS), 542 (having the sequence of KDS), 550 (having the sequence of DAS), 558 (having the sequence of AAS), 566 (having the sequence of GAS), 574 (having the sequence of DDS), 582 (having the sequence of KDS), 590 (having the sequence of SAS), 598 (having the sequence of SAS), 606 (having the sequence of GAS), 614 (having the sequence of GAS), 622 (having the sequence of GAS), 644 (having the sequence of GAS), 652 (having the sequence of SAS), 661 (having the sequence of GAS) and 669 (having the sequence of DAS); and the CDR3 of the second light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:41, 49, 57, 65, 73, 169, 177, 185, 262, 270, 278, 286, 519, 527, 535, 543, 551, 559, 567, 575, 583, 591, 599, 607, 615, 623, 645, 653, 662 and 670. In some preferred aspects, CDR1, CDR2 and CDR3 of each of the first heavy chain variable region, the second heavy chain variable region, the first light chain variable region and the second light chain variable region have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of the first heavy chain variable region, the second heavy chain variable region, the first light chain variable region and the second light chain variable region comprise or consist of the corresponding amino acid sequences specified above.

In some aspects, the first heavy chain variable domain comprises an amino acid sequence having at least 80%, at least 85%, at least 90%, at least 91%, at least 92%, at least 93%, at least 94%, at least 95%, at least 96%, at least 97%, at least 98%, or 100% identity to any one of SEQ ID NOs:42, 50, 58, 66, 74, 170, 178, 186, 263, 271, 279, 287, 510, 512, 520, 528, 536, 544, 552, 560, 568, 576, 584, 592, 600, 608, 616, 624, 626, 628, 630, 632, 634, 636, 638, 646, 654, 663, 779, 781, 785 and 787; the first light chain variable domain comprises an amino acid sequence having at least 80%, at least 85%, at least 90%, at least 91%, at least 92%, at least 93%, at least 94%, at least 95%, at least 96%, at least 97%, at least 98%, or 100% identity to any one of SEQ ID NOs:38, 46, 54, 62, 70, 166, 174, 182, 259, 267, 275, 283, 508, 509, 511, 516, 524, 532, 540, 548, 556, 564, 572, 580, 588, 596, 604, 612, 620, 625, 627, 629, 631, 633, 635, 637, 642, 650, 659 and 667; the second heavy chain variable domain comprises an amino acid sequence having at least 80%, at least 85%, at least 90%, at least 91%, at least 92%, at least 93%, at least 94%, at least 95%, at least 96%, at least 97%, at least 98%, or 100% identity to any one of SEQ ID NOs:42, 50, 58, 66, 74, 170, 178, 186, 263, 271, 279, 287, 510, 512, 520, 528, 536, 544, 552, 560, 568, 576, 584, 592, 600, 608, 616, 624, 626, 628, 630, 632, 634, 636, 638, 646, 654, 663, 779, 781, 785 and 787; and the second light chain variable domain comprises an amino acid sequence having at least 80%, at least 85%, at least 90%, at least 91%, at least 92%, at least 93%, at least 94%, at least 95%, at least 96%, at least 97%, at least 98%, or 100% identity to any one of SEQ ID NOs:38, 46, 54, 62, 70, 166, 174, 182, 259, 267, 275, 283, 508, 509, 511, 516, 524, 532, 540, 548, 556, 564, 572, 580, 588, 596, 604, 612, 620, 625, 627, 629, 631, 633, 635, 637, 642, 650, 659 and 667. In some aspects, the sequence has a mutation at N62, for example, N62Q or N62Q at, for example, SEQ ID NO:279. In some preferred aspects, the first heavy chain variable region, the second heavy chain variable region, the first light chain variable region and the second light chain variable region have at least 90% identity with the corresponding amino acid sequences specified above, optionally a mutation at N62 as recited above. As used herein, “at least 90% identity” includes at least 91%, 92%, 93%, 94%, 95%, 96%, 97%, 98%, 99% and 100% identity to the recited reference sequence. In some particularly preferred aspects, the first heavy chain variable region, the second heavy chain variable region, the first light chain variable region and the second light chain variable region comprise or consist of the corresponding amino acid sequences specified above, optionally with a mutation at N62 as specified above.

In some aspects, the anti-SARS-CoV2 multivalent antibody further comprises a third antigen binding domain.

In some aspects, the third antigen binding site is different than the first antigen binding site, the second antigen binding site, or both the first and second antigen binding sites.

In some aspects, the third antigen binding site comprises a third heavy chain variable domain and a third light chain variable domain.

In some aspects, the third heavy chain variable domain comprises an amino acid sequence having at least 80%, at least 85%, at least 90%, at least 91%, at least 92%, at least 93%, at least 94%, at least 95%, at least 96%, at least 97%, at least 98%, or 100% identity to any one of SEQ ID NOs:42, 50, 58, 66, 74, 170, 178, 186, 263, 271, 279, 287, 510, 512, 520, 528, 536, 544, 552, 560, 568, 576, 584, 592, 600, 608, 616, 624, 626, 628, 630, 632, 634, 636, 638, 646, 654, 663, 779, 781, 785 and 787; and the third light chain variable domain comprises an amino acid sequence having at least 80%, at least 85%, at least 90%, at least 91%, at least 92%, at least 93%, at least 94%, at least 95%, at least 96%, at least 97%, at least 98%, or 100% identity to any one of SEQ ID NOs:38, 46, 54, 62, 70, 166, 174, 182, 259, 267, 275, 283, 508, 509, 511, 516, 524, 532, 540, 548, 556, 564, 572, 580, 588, 596, 604, 612, 620, 625, 627, 629, 631, 633, 635, 637, 642, 650, 659 and 667. In some aspects, the sequence has a mutation at N62, for example, N62Q or N62Q at, for example, SEQ ID NO:279. In some preferred aspects, the third heavy chain variable region has at least 90% identity with the corresponding amino acid sequences specified above, optionally with a mutation at N62 as specified above. As used herein, “at least 90% identity” includes at least 91%, 92%, 93%, 94%, 95%, 96%, 97%, 98%, 99% and 100% identity to the recited reference sequence. In some particularly preferred aspects, the third heavy chain variable region comprises or consists of the corresponding amino acid sequences specified above, optionally with a mutation at N62 as specified above.

In some aspects, the third heavy chain variable region comprises CDR1, CDR2 and CDR3 regions.

In some aspects, the CDR1 of the third heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664; the CDR2 of the third heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; the CDR3 of the third heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666; the CDR1 of the third light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:39, 47, 55, 63, 71, 167, 175, 183, 260, 268, 276, 284, 517, 525, 533, 541, 549, 557, 565, 573, 581, 589, 597, 605, 613, 621, 643, 651, 660 and 668; the CDR2 of the third light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:40, 48, 56, 64, 72, 168, 176, 184, 261, 269, 277 (having the sequence of LGS), 285, 518 (having the sequence of DAS), 526 (having the sequence of DVS), 534 (having the sequence of EDS), 542 (having the sequence of KDS), 550 (having the sequence of DAS), 558 (having the sequence of AAS), 566 (having the sequence of GAS), 574 (having the sequence of DDS), 582 (having the sequence of KDS), 590 (having the sequence of SAS), 598 (having the sequence of SAS), 606 (having the sequence of GAS), 614 (having the sequence of GAS), 622 (having the sequence of GAS), 644 (having the sequence of GAS), 652 (having the sequence of SAS), 661 (having the sequence of GAS) and 669 (having the sequence of DAS); and the CDR3 of the third light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:41, 49, 57, 65, 73, 169, 177, 185, 262, 270, 278, 286, 519, 527, 535, 543, 551, 559, 567, 575, 583, 591, 599, 607, 615, 623, 645, 653, 662 and 670. In some preferred aspects, CDR1, CDR2 and CDR3 of the third heavy chain variable region and the third light chain variable region have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of third heavy chain variable region and the third light chain variable region comprise or consist of the corresponding amino acid sequences specified above.

In some aspects, the anti-SARS-CoV2 multivalent antibody further comprises a fourth antigen binding domain.

In some aspects, the fourth antigen binding site is different than one or more of the first, second and third antigen binding sites.

In some aspects, the first, second, third and fourth antigen binding sites are different.

In some aspects, the fourth antigen binding site comprises a third heavy chain variable domain and a fourth light chain variable domain.

In some aspects, the fourth heavy chain variable domain comprises an amino acid sequence having at least 80%, at least 85%, at least 90%, at least 91%, at least 92%, at least 93%, at least 94%, at least 95%, at least 96%, at least 97%, at least 98%, or 100% identity to any one of SEQ ID NOs:42, 50, 58, 66, 74, 170, 178, 186, 263, 271, 279, 287, 510, 512, 520, 528, 536, 544, 552, 560, 568, 576, 584, 592, 600, 608, 616, 624, 626, 628, 630, 632, 634, 636, 638, 646, 654, 663, 779, 781, 785 and 787; and the fourth light chain variable domain comprises an amino acid sequence having at least 80%, at least 85%, at least 90%, at least 91%, at least 92%, at least 93%, at least 94%, at least 95%, at least 96%, at least 97%, at least 98%, or 100% identity to any one of SEQ ID NOs:38, 46, 54, 62, 70, 166, 174, 182, 259, 267, 275, 283, 508, 509, 511, 516, 524, 532, 540, 548, 556, 564, 572, 580, 588, 596, 604, 612, 620, 625, 627, 629, 631, 633, 635, 637, 642, 650, 659 and 667. In some aspects, the sequence has a mutation at N62, for example, N62Q or N62Q at, for example, SEQ ID NO:279. In some preferred aspects, the fourth heavy chain variable region has at least 90% identity with the corresponding amino acid sequences specified above, optionally with a mutation at N62 as recited above. As used herein, “at least 90% identity” includes at least 91%, 92%, 93%, 94%, 95%, 96%, 97%, 98%, 99% and 100% identity to the recited reference sequence. In some particularly preferred aspects, the fourth heavy chain variable region comprises or consists of the corresponding amino acid sequences specified above, optionally with a mutation at N62 as specified above.

In some aspects, the fourth heavy chain variable region comprises CDR1, CDR2 and CDR3 regions.

In some aspects, the CDR1 of the fourth heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:43, 51, 59, 67, 75, 171, 179, 187, 264, 272, 280, 288, 513, 521, 529, 537, 545, 553, 561, 569, 577, 585, 593, 601, 609, 617, 639, 647, 656 and 664; the CDR2 of the fourth heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:44, 52, 60, 68, 76, 172, 180, 188, 265, 273, 281, 289, 514, 522, 530, 538, 546, 554, 562, 570, 578, 586, 594, 602, 610, 618, 640, 648, 657 and 665; the CDR3 of the fourth heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:45, 53, 61, 69, 77, 173, 181, 189, 266, 274, 282, 290, 515, 523, 531, 539, 547, 555, 563, 571, 579, 587, 595, 603, 611, 619, 641, 649, 658 and 666; the CDR1 of the fourth light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:39, 47, 55, 63, 71, 167, 175, 183, 260, 268, 276, 284, 517, 525, 533, 541, 549, 557, 565, 573, 581, 589, 597, 605, 613, 621, 643, 651, 660 and 668; the CDR2 of the fourth light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:40, 48, 56, 64, 72, 168, 176, 184, 261, 269, 277 (having the sequence of LGS), 285, 518 (having the sequence of DAS), 526 (having the sequence of DVS), 534 (having the sequence of EDS), 542 (having the sequence of KDS), 550 (having the sequence of DAS), 558 (having the sequence of AAS), 566 (having the sequence of GAS), 574 (having the sequence of DDS), 582 (having the sequence of KDS), 590 (having the sequence of SAS), 598 (having the sequence of SAS), 606 (having the sequence of GAS), 614 (having the sequence of GAS), 622 (having the sequence of GAS), 644 (having the sequence of GAS), 652 (having the sequence of SAS), 661 (having the sequence of GAS) and 669 (having the sequence of DAS); and the CDR3 of the fourth light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:41, 49, 57, 65, 73, 169, 177, 185, 262, 270, 278, 286, 519, 527, 535, 543, 551, 559, 567, 575, 583, 591, 599, 607, 615, 623, 645, 653, 662 and 670. In some preferred aspects, CDR1, CDR2 and CDR3 of the fourth heavy chain variable region and the fourth light chain variable region have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of fourth heavy chain variable region and the fourth light chain variable region comprise or consist of the corresponding amino acid sequences specified above.

In some aspects of any of the antigen binding polypeptides or antigen binding polypeptide complexes provided herein, one or more VL (e.g., VL1, VL2, VL3 and/or VL4) comprises an amino acid sequence having at least 80%, at least 85%, at least 90%, at least 95%, at least 99%, or 100% identity to a peptide in the expression vector of SEQ ID NO:783. In some aspects, such a polypeptide or polypeptide complex further comprises one or more VH (e.g., VH1, VH2, VH3 and/or VH4) that comprises an amino acid having at least 80%, at least 85%, at least 90%, at least 95%, at least 99%, or 100% identity to any one of SEQ ID NOs:279, 779, 781, 785 and 787.

In some aspects of any of the antigen binding polypeptides or antigen binding polypeptide complexes provided herein, one or more VL (e.g., VL1, VL2, VL3 and/or VL4) is encoded by a polynucleotide sequence having at least 80%, at least 85%, at least 90%, at least 95%, at least 99%, or 100% identity to SEQ ID NO:784. In some aspects, such a polypeptide or polypeptide complex further comprises one or more VH (e.g., VH1, VH2, VH3 and/or V14) that comprises an amino acid having at least 80%, at least 85%, at least 90%, at least 95%, at least 99%, or 100% identity to any one of SEQ ID NOs:279, 779, 781, 785 and 787.

In some aspects of any of the antigen binding polypeptides or antigen binding polypeptide complexes provided herein, one or more VH (e.g., VH1, VH2, VH3 and/or V14) is encoded by a polynucleotide sequence having at least 80%, at least 85%, at least 90%, at least 95%, at least 99%, or 100% identity to SEQ ID NO:778. In some aspects, such a polypeptide or polypeptide complex further comprises one or more VL (e.g., VL1, VL2, VL3 and/or VL4) that comprises an amino acid having at least 80%, at least 85%, at least 90%, at least 95%, at least 99%, or 100% identity to SEQ ID NO:275.

In some aspects of any of the antigen binding polypeptides or antigen binding polypeptide complexes provided herein, one or more VH (e.g., VH1, VH2, VH3 and/or V14) comprises an amino acid sequence having at least 80%, at least 85%, at least 90%, at least 95%, at least 99%, or 100% identity to SEQ ID NO:779. In some aspects, such a polypeptide or polypeptide complex further comprises one or more VL (e.g., VL1, VL2, VL3 and/or VL4) that comprises an amino acid having at least 80%, at least 85%, at least 90%, at least 95%, at least 99%, or 100% identity to SEQ ID NO:275.

In some aspects of any of the antigen binding polypeptides or antigen binding polypeptide complexes provided herein, one or more VH (e.g., VH1, VH2, VH3 and/or V14) is encoded by a polynucleotide having at least 80%, at least 85%, at least 90%, at least 95%, at least 99%, or 100% identity to SEQ ID NO:780. In some aspects, such a polypeptide or polypeptide complex further comprises one or more VL (e.g., VL1, VL2, VL3 and/or VL4) that comprises an amino acid having at least 80%, at least 85%, at least 90%, at least 95%, at least 99%, or 100% identity to SEQ ID NO:275.

In some aspects of any of the antigen binding polypeptides or antigen binding polypeptide complexes provided herein, one or more VH (e.g., VH1, VH2, VH3 and/or V14) comprises an amino acid sequence having at least 80%, at least 85%, at least 90%, at least 95%, at least 99%, or 100% identity to SEQ ID NO:781. In some aspects, such a polypeptide or polypeptide complex further comprises one or more VL (e.g., VL1, VL2, VL3 and/or VL4) that comprises an amino acid having at least 80%, at least 85%, at least 90%, at least 95%, at least 99%, or 100% identity to SEQ ID NO:275.

In some aspects of any of the antigen binding polypeptides or antigen binding polypeptide complexes provided herein, one or more VH (e.g., VH1, VH2, VH3 and/or V14) is encoded by a polynucleotide having at least 80%, at least 85%, at least 90%, at least 95%, at least 99%, or 100% identity to SEQ ID NO:782. In some aspects, such a polypeptide or polypeptide complex further comprises one or more VL (e.g., VL1, VL2, VL3 and/or VL4) that comprises an amino acid having at least 80%, at least 85%, at least 90%, at least 95%, at least 99%, or 100% identity to SEQ ID NO:275.

In some aspects of any of the antigen binding polypeptides or antigen binding polypeptide complexes provided herein, one or more VH (e.g., VH1, VH2, VH3 and/or V14) comprises an amino acid sequence having at least 80%, at least 85%, at least 90%, at least 95%, at least 99%, or 100% identity to a peptide in the expression vector of SEQ ID NO:785. In some aspects, such a polypeptide or polypeptide complex further comprises one or more VL (e.g., VL1, VL2, VL3 and/or VL4) that comprises an amino acid having at least 80%, at least 85%, at least 90%, at least 95%, at least 99%, or 100% identity to SEQ ID NO:275.

In some aspects of any of the antigen binding polypeptides or antigen binding polypeptide complexes provided herein, one or more VH (e.g., VH1, VH2, VH3 and/or V14) is encoded by a polynucleotide having at least 80%, at least 85%, at least 90%, at least 95%, at least 99%, or 100% identity to SEQ ID NO:786. In some aspects, such a polypeptide or polypeptide complex further comprises one or more VL (e.g., VL1, VL2, VL3 and/or VL4) that comprises an amino acid having at least 80%, at least 85%, at least 90%, at least 95%, at least 99%, or 100% identity to SEQ ID NO:275.

In some aspects of any of the antigen binding polypeptides or antigen binding polypeptide complexes provided herein, one or more VH (e.g., VH1, VH2, VH3 and/or V14) comprises an amino acid sequence having at least 80%, at least 85%, at least 90%, at least 95%, at least 99%, or 100% identity to a peptide in the expression vector of SEQ ID NO:787. In some aspects, such a polypeptide or polypeptide complex further comprises one or more VL (e.g., VL1, VL2, VL3 and/or VL4) that comprises an amino acid having at least 80%, at least 85%, at least 90%, at least 95%, at least 99%, or 100% identity to SEQ ID NO:275.

In some aspects of any of the antigen binding polypeptides or antigen binding polypeptide complexes provided herein, one or more VH (e.g., VH1, VH2, VH3 and/or V14) is encoded by a polynucleotide having at least 80%, at least 85%, at least 90%, at least 95%, at least 99%, or 100% identity to SEQ ID NO:788. In some aspects, such a polypeptide or polypeptide complex further comprises one or more VL (e.g., VL1, VL2, VL3 and/or VL4) that comprises an amino acid having at least 80%, at least 85%, at least 90%, at least 95%, at least 99%, or 100% identity to SEQ ID NO:275.

In some aspects, the anti-SARS-CoV2 multivalent antibody is any antibody or antigen binding fragment thereof described herein.

In some aspects, the antigen is the SARS-CoV-2 protein. In some aspects, the SARS-CoV-2 protein is a membrane protein, nucleocapsid protein, envelope protein, or spike protein, or a combination thereof. In some aspects, the SARS-CoV-2 protein is a spike protein.

In some aspects, the SARS-CoV2 virus is a SARS-CoV2 variant.

In some aspects, the SARS-CoV2 variant is Alpha variant (B.1.1.7), Beta variant (B.1.351), Gamma variant (P.1), Delta variant, Epsilon variant (B.1.427), or Omicron variant (B.1.1.529), or a combination thereof.

In some aspects, the Omicron variant is BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, CH.1.1, or a combination thereof.

In some aspects, the method further comprises administering to the subject an effective amount of one or more additional pharmaceutical agents (e.g., 1, 2, 3, 4, 5 or more). In some aspects, the method further comprises administering to the subject an effective amount of an additional pharmaceutical agent. In some aspects, treating or preventing a disease as disclosed herein further comprises administering to the subject an effective amount of one or more additional pharmaceutical agents (e.g., 1, 2, 3, 4, 5 or more). In some aspects, treating or preventing a disease as disclosed herein further comprises administering to the subject an effective amount of an additional pharmaceutical agent.

In some aspects, the method further comprises administering to the subject an effective amount of one or more pharmaceutical compositions comprising (i) an additional pharmaceutical agent and (ii) a pharmaceutically acceptable carrier (e.g., 1, 2, 3, 4, 5 or more). In some aspects, the method further comprises administering to the subject an effective amount of a pharmaceutical composition comprising (i) an additional pharmaceutical agent and (ii) a pharmaceutically acceptable carrier. In some aspects, treating or preventing a disease as disclosed herein further comprises administering to the subject an effective amount of one or more pharmaceutical compositions comprising (i) an additional pharmaceutical agent and (ii) a pharmaceutically acceptable carrier (e.g., 1, 2, 3, 4, 5 or more). In some aspects, treating or preventing a disease as disclosed herein further comprises administering to the subject an effective amount of a pharmaceutical composition comprising (i) an additional pharmaceutical agent and (ii) a pharmaceutically acceptable carrier.

In some aspects, the additional pharmaceutical agent is 25-hydroxyvitamin D, an agent that potentiates vitamin D action, an anti-viral agent, an anti-malarial agent, an antibiotic, or a combination thereof.

In some aspects, the additional pharmaceutical agent is 25-hydroxyvitamin D.

In some aspects, the agent that potentiates vitamin D action is a CYP24 inhibitor, 1,25-dihydroxyvitamin D compound, or a combination thereof.

In some aspects, the anti-viral agent is an anti-retroviral agent, an antibody against SARS-CoV-2 virus, an inhibitor of reverse transcriptase, or a combination thereof.

In some aspects, the anti-viral agent is maraviroc, enfuvirtide, amantadine, lamivudine, nevirapine, efavirenz, dolutegravir, elvitegravir, raltegravir, acyclovir and any nucleoside analog of aciclovir, ganciclovir, cidofovir, forcarnet, ribavirin, interferon alpha, pegylated interferon alpha, boceprevir, atazanavir, darunavir, indinavir, oseltamivir, zanamivir, rimantadine, peremivir, valaciclovir, penciclovir, valganciclovir, foscarnet, tenofovir, adefovir, entecavir, lamivudine, telbivudine, ribavirin, glecaprevir, grazoprevir, paritaprevir, simeprevir, voxilaprevir, daclatasvir, elbasvir, ledipasvir, ombitasvir, pibrentasvir, velpatasvir, dasabuvir, famciclovir, remdesivir, trifluridine, sofobuvir, bebtelovimab, or a combination thereof.

In some aspects, the anti-viral agent is bebtelovimab.

In some aspects, the anti-viral agent is Retrovir® (3′-azido-3′-deoxypyrimidine, zidovudine), 3′-azido-3′-deoxythymidine (AZT), HMD® (2′,3′-dideoxycytidine, zalcitabine), VidexEC® (2′,3′dideoxyinosine, didanosine), Epivir® (lamivudine), Zerit® (stavudine), Viread® (tenofovir DF), Ziagen® (abacavir), Emtriva® (emtricitabine, FTC), Rescriptor® (delavirdine), Sustiva® (efavirenz), Viramune® (nevirapine, 11-cyclopropyl-4-methyl-5,11-dihydro-6H-dipyrido[3,2-b:2′,3′-e][1,4]diazepin-6-one), trisodium phosphonoformate, ammonium-21-tungstenato-9-antimonate, 1-β-D-ribofuranoxyl-1,2,4-triazole-3-carboxamide, Aganerase® (amprenavir), Reyataz® (atazanavir), Lexiva® (fosamprenavir), Crixivan® (indinavir), Viracept® (nelfinavir), Norvir® (ritonavir), Fortovase® or Invirase® (saquinavir), lasinavir (5(S)-(tert-butoxycarbonylamino)-4(S)-hydroxy-6-phenyl-2(R)(2,3,4-trimethoxyphenylmethyl)-hexanoyl-(L)-valyl-N-(2-metoxy-ethyl)-amide), adriamycin, KVX-478, VX-478, 141W94, AG-1343, KNI-272, U-96988, BILA-2011 BS (palinavir), polymannoacetate, Fuzeon® (enfuvirtide, T-20), Epzicom® (abacavir and lamivudine), Trizivir® (abacavir, lamivudine and zidovudine), Truvada® (emtricitabine and tenofir DF), Combivir® (lamivudine and zidovudine), Kaletra® (lopinavir and ritonavir), bebtelovimab, or a combination thereof.

In some aspects, the additional pharmaceutical agent is administered before the antigen binding polypeptide, antigen binding polypeptide complex, antibody or antigen binding fragment thereof.

In some aspects, the additional pharmaceutical agent is administered after the antigen binding polypeptide, antigen binding polypeptide complex, antibody or antigen binding fragment thereof.

In some aspects, the additional pharmaceutical agent is administered concurrently with the antigen binding polypeptide, antigen binding polypeptide complex, antibody or antigen binding fragment thereof.

In some aspects, the additional pharmaceutical agent and the antigen binding polypeptide, antigen binding polypeptide complex, antibody or antigen binding fragment thereof are administered in the same pharmaceutical composition.

In some aspects, the additional pharmaceutical agent and the antigen binding polypeptide, antigen binding polypeptide complex, antibody or antigen binding fragment thereof are administered in different pharmaceutical compositions.

### EXAMPLES

The following examples are provided to further illustrate aspects of the disclosure, and are not meant to constrain the disclosure to any particular application or theory of operation.

### Example 1

### Synthesis, Cloning and Expression of Multispecific Antibodies

After design of the amino acid sequences for each multispecific antibody, the genes for each multispecific antibody were synthesized using human preferred codons (GeneArt) and cloned into eukaryotic expression vectors. For each multispecific antibody expression, the single plasmid DNA (bispecific) and equal amounts of the two plasmid DNAs (trispecific) were transfected into Expi293F cells (Thermo Fisher Scientific) using PEI MAX (Polysciences). The transfected cells were cultured in a shaker incubator at 120 rpm, 37° C., 9% CO2 for 5 days. Culture supernatants were harvested and filtered, the multispecific antibodies were purified over a Protein A (GE Health Science) column. Each multispecific antibody was eluted with Pierce IgG Elution Buffer, pH 2.0 (Thermo Fisher Scientific), immediately buffer exchanged with Histidine pH 6.0 and concentrated using Amicon Ultra-15 Centrifugal Filter Units (MilliporeSigma). After concentration, each multispecific antibody was applied to a Superdex 200 16/600 size exclusion column (Cytiva) to remove aggregates and different species in the preparation. The fractions were then analyzed on reduced and non-reduced sodium dodecyl sulfate-polyacrylamide gel electrophoresis (SDS-PAGE) to identify the fractions that contained the monomeric multispecific antibody before combining them. The pooled fractions were then further concentrated, aliquoted, and analyzed by SDS-PAGE as well as an analytical size exclusion chromatography (SEC) column (Superdex 200 16/600) to verify purity.

FIG. 1A-1D shows exemplary configurations of monospecific, tetravalent molecules made. FIG. 1A: monospecific, tetravalent molecule containing VH and VL of the anti-SARS-CoV-2 antibody, B1-182, with Fc region (MX241). FIG. 1B: monospecific, tetravalent molecule containing VH and VL of B1-182, with Fc, CL and CH1 (MX365). FIG. 1C: monospecific, tetravalent molecule containing VH and VL of the anti-SARS-CoV-2 hybrid antibody, A23-58H/B1-182L, with Fc (MX409). FIG. 1D: monospecific, tetravalent molecule containing VH and VL of A23-58H/B1-182L, with Fc, CL and CH1 (MX466).

FIG. 2A-2P shows exemplary configurations of bispecific, tetravalent molecules made. FIG. 2A: bispecific, tetravalent molecule containing VH and VL of B1-182 and VH and VL of the anti-SARS-CoV-2 antibody, A19-46, with Fc (MX179). FIG. 2B: bispecific, tetravalent molecule containing VH and VL of B1-182 and VH and VL of A19-46, with Fc (MX180). FIG. 2C: bispecific, tetravalent molecule containing VH and VL of B1-182 antibody and VH and VL of the anti-SARS-CoV-2 antibody, A19-61, with Fc (MX183). FIG. 2D: bispecific, tetravalent molecule containing VH and VL of B1-182 and VH and VL of A19-61, with Fc (MX184). FIG. 2E: bispecific, tetravalent molecule containing VH and VL of B1-182 and VH and VL of A19-61, with Fc, CL and CH1 (MX366). FIG. 2F: bispecific, tetravalent molecule containing VH and VL of B1-182 and VH and VL of A19-61, with Fc, CL and CH1 (MX367). FIG. 2G: bispecific, tetravalent molecule containing VH and VL of A23-58H/B1-182L and VH and VL of A19-61, with Fc (MX412). FIG. 2H: bispecific, tetravalent molecule containing VH and VL of A23-58H/B1-182L and VH and VL of A19-61, with Fc (MX413). FIG. 2I: bispecific, tetravalent molecule containing VH and VL of A23-58H/B1-182L and VH and VL of A19-46, with Fc (MX490). FIG. 2J: bispecific, tetravalent molecule containing VH and VL of A23-58H/B1-182L and VH and VL of A19-46, with Fc (MX492). FIG. 2K: bispecific, tetravalent molecule containing VH and VL of A23-58H/B1-182L hybrid antibody and VH and VL of A19-61 antibody, with Fc, CL and CH1 (MX467). FIG. 2L: bispecific, tetravalent molecule containing VH and VL of A23-58H/B1-182L and VH and VL of A19-61, with Fc, CL and CH1 (MX468). FIG. 2M: bispecific, tetravalent molecule containing VH and VL of A23-58H/B1-182L and VH and VL of A19-46, with Fc, CL and CH1 (MX491). FIG. 2N: bispecific, tetravalent molecule containing VH and VL of A23-58H/B1-182L and VH and VL of A19-46, with Fc, CL and CH1 (MX493). FIG. 2O: bispecific, tetravalent molecule containing VH and VL of the anti-SARS-CoV-2 antibody, A23-58, and VH and VL of A19-61 (MX326). FIG. 2P: bispecific, tetravalent molecule containing VH and VL of A23-58 and VH and VL of A19-61 (MX327).

FIG. 3A-3H shows exemplary configurations of trispecific, tetravalent molecules made. FIG. 3A: trispecific, tetravalent molecule containing VH and VL of A19-46 antibody, VH and VL of A19-61 antibody, and VH and VL of B1-182 antibody with Fc and knob-into-hole modification (MX494). FIG. 3B: trispecific, tetravalent molecule containing VH and VL of A19-46 antibody, VH and VL of A19-61 antibody, and VH and VL of B1-182 antibody with Fc and knob-into-hole modification (MX495). FIG. 3C: trispecific, tetravalent molecule containing VH and VL of A19-46 antibody, VH and VL of A19-61 antibody, and VH and VL of B1-182 antibody with Fc and knob-into-hole modification (MX496). FIG. 3D: trispecific, tetravalent molecule containing VH and VL of A19-46 antibody, VH and VL of A19-61 antibody, and VH and VL of B1-182 antibody with Fc and knob-into-hole modification (MX497). FIG. 3E: trispecific, tetravalent molecule containing VH and VL of A19-46 antibody, VH and VL of A19-61 antibody, and VH and VL of A23-58H/B1-182L hybrid antibody with Fc and knob-into-hole modification (MX498). FIG. 3F: trispecific, tetravalent molecule containing VH and VL of A19-46 antibody, VH and VL of A19-61 antibody, and VH and VL of A23-58H/B1-182L hybrid antibody with Fc and knob-into-hole modification (MX499). FIG. 3G: trispecific, tetravalent molecule containing VH and VL of A19-46 antibody, VH and VL of A19-61 antibody, and VH and VL of A23-58H/B1-182L hybrid antibody with Fc and knob-into-hole modification (MX500). FIG. 3H: trispecific, tetravalent molecule containing VH and VL of A19-46 antibody, VH and VL of A19-61 antibody, and VH and VL of A23-58H/B1-182L hybrid antibody with Fc and knob-into-hole modification (MX501).

FIG. 11 shows exemplary configurations of tetravalent, bispecific and tetravalent, trispecific molecules made. MX828 contains two VH/VL pairs of A19-46 and two VH/VL pairs of A23-58H/A23-58L93VGLTG. MX829 contains two VH/VL pairs of A19-61 and two VH/VL pairs of A23-58H/A23-58L93VGLTG. MX830 contains two VH/VL pairs of A19-46 and two VH/VL pairs of A23-58H/A23-58L94GLTG. MX831 contains two VH/VL pairs of A19-61 and two VH/VL pairs of A23-58H/A23-58L94GLTG. MX834 and MX837 contain one VH/VL pair of A19-46, one VH/VL pair of A19-61, and two VH/VL pairs of A23-58H/A23-58L93VGLTG. MX835 and MX838 contain one VH/VL pair of A19-46, one VH/VL pair of A19-61, and two VH/VL pairs of A23-58H/A23-58L94GLTG.

### Example 2

### Pseudovirus Neutralization Assay

The pseudotyped neutralization potency (IC50 and IC80) of multispecific antibodies described in Example 1 was then determined. Specifically, S-containing lentiviral pseudovirions were produced by co-transfection of packaging plasmid pCMVAR8.2, transducing plasmid pHR′ CMV-Luc, a TMPRSS2 plasmid, and S plasmids from SARS-CoV-2 variants into Lenti-X 293T Cell Line (Takara) using FuGENE 6 Transfection Reagent (Promega). Human ACE2 293T Cell Line (Takara) were plated into 96-well Flat Clear Bottom White Polystyrene TC-treated Microplates (Corning) at 75,00 cells per well the day before infection of SARS CoV-2 pseudovirus. Serial dilutions of multispecific antibodies were mixed with titrated pseudovirus, incubated for 45 minutes at 37° C. and added to cells in triplicate. Cells were lysed 72 h later, and luciferase activity was measured with Varioskan LUX Multimode Microplate Reader (Thermo Fisher Scientific). Percent neutralization and neutralization IC50 and IC80 were calculated using GraphPad Prism 9.3.1.

Tables 2-7 show the IC50 and IC80 values (in nM) of multispecific antibodies described in Example 1 for neutralization of SARS-CoV-2 original Wuhan strain (WT), D614G spike protein mutant (D614G), Alpha variant (B.1.1.7), Beta variant 1 (B.1.351 v1), Gamma variant (P.1), Kappa variant (B.1.617.1) Delta variants (B.1.617.2 and AY.1), Iota variant (B.1.526), Lambda variant (C.37) Mu variant (B.1.621), C.1.2 variant, Omicron variants (BA.1, BA.2, BA.2.12.1, and BA.4/5). IC50 and IC80 values for the A23-58, B1-182, A23-58H/B1-182L hybrid, A19-46, and A19-61 parental antibodies are also shown.

TABLE 2

| | WT | | D614G | | B.1.1.7 | |
|---|---|---|---|---|---|---|
| Name | IC50 | IC80 | IC50 | IC80 | IC50 | IC80 |
| A23-58 mAb | | | 0.0185 | 0.0477 | 0.0329 | 0.1041 |
| B1-182 mAb | 0.0264 | 0.0692 | 0.0171 | 0.0359 | 0.0262 | 0.0844 |
| A23-58H/B1-182L mAb | | | 0.0132 | 0.0337 | 0.0174 | 0.0376 |
| A19-61 mAb | 0.1725 | 0.4078 | 0.0550 | 0.1258 | 0.1747 | 0.3251 |
| A19-46 mAb | 0.2633 | 0.7783 | 0.1379 | 0.3893 | 0.1899 | 0.4859 |
| MX241 (B1-182 Tetravalent) | | | 0.0045 | 0.0134 | 0.0175 | 0.0419 |
| MX409 (A23-58H/B1-182L Tetravalent) | | | 0.0051 | 0.0113 | 0.0139 | 0.0286 |
| MX183 (A19-61 × B1-182) | | | 0.7571 | 1.6249 | 0.8030 | 5.6644 |
| MX184 (B1-182 × A19-61) | | | 0.0196 | 0.0597 | 0.0772 | 0.1856 |
| MX412 (A23-58H/B1-182L × A19-61) | | | 0.0294 | 0.0681 | 0.0580 | 0.1432 |
| MX413 (A19-61 × A23-58H/B1-182L) | | | 0.0193 | 0.0479 | 0.0547 | 0.1408 |

TABLE 3

| | B.1.351.v1 | | P.1 | | B.1.617.2 | |
|---|---|---|---|---|---|---|
| Name | IC50 | IC80 | IC50 | IC80 | IC50 | IC80 |
| A23-58 mAb | 0.1227 | 0.3389 | 0.0097 | 0.0437 | 0.0133 | 0.0358 |
| B1-182 mAb | 0.0357 | 0.1390 | 0.0182 | 0.0429 | 0.0099 | 0.0254 |
| A23-58H/B1-182L mAb | 0.0305 | 0.0966 | 0.0116 | 0.0237 | 0.0104 | 0.0205 |
| A19-61 mAb | 0.0686 | 0.1127 | 0.1352 | 0.2885 | 0.2090 | 0.2917 |
| A19-46 mAb | 0.8074 | 2.0716 | 0.2600 | 0.7000 | >66.7 | >66.7 |
| MX241 (B1-182 Tetravalent) | 0.0133 | 0.0434 | 0.004712 | 0.013749 | 0.004467 | 0.013909 |
| MX409 (A23-58H/B1-182L Tetravalent) | 0.0073 | 0.0175 | 0.001817 | 0.00565 | 0.003127 | 0.008822 |
| MX183 (A19-61 × B1-182) | 1.0235 | 1.6494 | 0.429548 | 2.265246 | 0.229678 | 0.947623 |
| MX184 (B1-182 × A19-61) | 0.0441 | 0.1200 | 0.032549 | 0.13742 | 0.012894 | 0.045828 |
| MX412 (A23-58H/B1-182L × A19-61) | 0.0365 | 0.0840 | 0.041954 | 0.088517 | 0.027924 | 0.06254 |
| MX413 (A19-61 × A23-58H/B1-182L) | 0.0386 | 0.0920 | 0.025005 | 0.054702 | 0.019271 | 0.06548 |

TABLE 4

| | B.1.617.1 | | B.1.526 | | C.37 | |
|---|---|---|---|---|---|---|
| Name | IC50 | IC80 | IC50 | IC80 | IC50 | IC80 |
| A23-58 mAb | | | | | | |
| B1-182 mAb | 0.0261 | 0.0860 | 0.0639 | 0.1831 | 0.0049 | 0.0150 |
| A23-58H/B1-182L mAb | | | | | | |
| A19-61 mAb | 0.0815 | 0.1732 | 0.0418 | 0.0836 | 0.1099 | 0.1865 |
| A19-46 mAb | >66.7 | >66.7 | 0.8033 | 1.8820 | >66.7 | >66.7 |

TABLE 5

| | B.1.621 | | C.1.2 | | AY.1 | |
|---|---|---|---|---|---|---|
| Name | IC50 | IC80 | IC50 | IC80 | IC50 | IC80 |
| A23-58 mAb | 0.1240 | 0.2600 | 0.0287 | 0.0656 | | |
| B1-182 mAb | 0.0695 | 0.2188 | 0.0549 | 0.1096 | 0.0080 | 0.0192 |
| A23-58H/B1-182L mAb | 0.0402 | 0.1081 | 0.0207 | 0.0348 | | |
| A19-61 mAb | 0.2639 | 0.4296 | 8.4254 | >66.7 | 0.0557 | 0.1207 |
| A19-46 mAb | 1.3387 | 3.8125 | 11.6200 | 21.0215 | >66.7 | >66.7 |
| MX241 (B1-182 Tetravalent) | 0.0184 | 0.0852 | 0.0039 | 0.0176 | 0.0035 | 0.0077 |
| MX409 (A23-58H/B1-182L Ttravalent) | 0.0046 | 0.0124 | 0.0050 | 0.0099 | | |
| MX183 (A19-61 × B1-182) | | | | | 0.5425 | 1.0383 |
| MX184 (B1-182 × A19-61) | | | | | 0.0174 | 0.0300 |
| MX412 (A23-58H/B1-182L × A19-61) | 0.5826 | 1.7763 | 0.0140 | 0.0528 | | |
| MX413 (A19-61 × A23-58H/B1-182L) | 0.0890 | 0.1661 | 0.0109 | 0.0260 | | |

TABLE 6

| | BA.1 | | BA.2 | | BA.2.12.1 | |
|---|---|---|---|---|---|---|
| Name | IC50 | IC80 | IC50 | IC80 | IC50 | IC80 |
| A23-58 mAb | 0.8529 | 4.2362 | 1.6769 | 5.6879 | 0.6471 | 4.9635 |
| B1-182 mAb | 1.0322 | 4.4388 | 2.3398 | 8.1854 | 0.7393 | 5.9249 |
| A23-58H/B1-182L mAb | 0.3638 | 1.3930 | 0.5437 | 2.4827 | 0.8298 | 3.2735 |
| A19-61 mAb | >66.7 | >66.7 | 0.7245 | 2.2411 | 0.1104 | 0.2980 |
| A19-46 mAb | 0.9580 | 2.2810 | 0.0645 | 0.1766 | >66.7 | >66.7 |
| MX241 (B1-182 Tetravalent) | 1.7089 | >6.7 | >6.7 | >6.7 | 5.6456 | >6.7 |
| MX409 (A23-58H/B1-182L Tetravalent) | 2.0254 | >66.7 | 4.9034 | >66.7 | 8.9648 | 52.7028 |
| MX183 (A19-61 × B1-182) | >66.7 | >66.7 | | | | |
| MX184 (B1-182 × A19-61) | 47.5701 | >66.7 | | | | |
| MX412 (A23-58H/B1-182L × A19-61) | 8.9636 | 31.8446 | 0.0304 | 0.0803 | 0.0615 | 0.1777 |
| MX413 (A19-61 × A23-58H/B1-182L) | 6.5827 | 20.5053 | 0.0141 | 0.0466 | 0.0149 | 0.0614 |
| MX466 (A23-58H/B1-182L Cl-CH1 Tetravalent) | 0.5703 | 8.2302 | 2.4675 | 44.2086 | 0.5863 | 18.0438 |
| MX467 (A23-58H/B1-182L × A19-61 CL-CH1) | 18.4909 | 69.3602 | 0.0373 | 0.0738 | 0.0181 | 0.1266 |
| MX468 (A19-61 × A23-58H/B1-182L CL-CH1) | 1.3331 | 4.0621 | 0.0289 | 0.0617 | 0.0224 | 0.0698 |
| MX179 (A19-46 × B1-182) | 0.0068 | 0.0193 | 0.0137 | 0.0389 | 0.1667 | 0.9290 |
| MX180 (B1-182 × A19-46) | 0.0187 | 0.0652 | 0.0410 | 0.1032 | 0.5839 | 2.8244 |
| MX494 (A19-61 × B1-182/A19-46 × B1-182) | 0.0227 | 0.0671 | 0.0053 | 0.0192 | 0.0248 | 0.0915 |
| MX495 (A19-61 × B1-182/B1-182 × A19-46) | 0.0780 | 0.1899 | 0.0191 | 0.0540 | 0.0513 | 0.0952 |
| MX496 (B1-182 × A19-61/A19-46 × B1-182) | 0.0907 | 0.2639 | 0.0231 | 0.0805 | 0.0975 | 0.2243 |
| MX497 (B1-182 × A19-61/B1-182 × A19-46) | 0.1087 | 0.4867 | 0.0251 | 0.0698 | 0.1298 | 0.2250 |
| MX 490 (A23-58H/B1-182L × A19-46) | 0.2195 | 0.4838 | 0.3920 | 0.8960 | >6.7 | >6.7 |
| MX491 (A23-58H/B1-182L × A19-46 CL-CH1) | 0.0316 | 0.0928 | 0.0489 | 0.1136 | 1.4370 | 7.8069 |
| MX492 (A19-46 × A23-58H/B1-182L) | 0.0058 | 0.0369 | 0.0201 | 0.0451 | 0.1069 | 0.9301 |
| MX493 (A19-46 × A23-58H/B1-182L CL-CH1) | 0.0158 | 0.0387 | 0.0200 | 0.0464 | 0.0331 | 0.0858 |
| MX498 (A23-58H/B1-182L × A19-46/A23-58H/B1-182L × A19-61) | 0.9724 | 2.0874 | 0.0654 | 0.1170 | 0.2043 | 0.3936 |
| MX499 (A23-58H/B1-182L × A19-46/A19-61 × A23-58H/B1-182L) | 0.2528 | 0.4946 | 0.0519 | 0.0824 | 0.0644 | 0.1202 |
| MX500 (A19-46 × A23-58H/B1-182L/A23-58H/B1-182L × A19-61) | 0.0639 | 0.2039 | 0.0151 | 0.0503 | 0.0655 | 0.1448 |
| MX501 (A19-46 × A23-58H/B1-182L/A19-61 × A23-58H/B1-182L) | 0.0285 | 0.0876 | 0.0097 | 0.0250 | 0.0405 | 0.0927 |

TABLE 7

| | BA.4/5 | |
|---|---|---|
| Name | IC50 | IC80 |
| A23-58 mAb | >66.7 | >66.7 |
| B1-182 mAb | >66.7 | >66.7 |
| A23-58H/B1-182L mAb | >66.7 | >66.7 |
| A19-61 mAb | 0.1282 | 0.3220 |
| A19-46 mAb | >66.7 | >66.7 |
| MX241 (B1-182 Tetravalent) | >6.7 | >6.7 |
| MX409 (A23-58H/B1-182L Tetravalent) | >66.7 | >66.7 |
| MX183 (A19-61 × B1-182) | | |
| MX184 (B1-182 × A19-61) | | |
| MX412 (A23-58H/B1-182L × A19-61) | 0.1863 | 0.3613 |
| MX413 (A19-61 × A23-58H/B1-182L) | 0.0572 | 0.1426 |
| MX466 (A23-58H/B1-182L Cl-CH1 Tetravalent) | >66.7 | >66.7 |
| MX467 (A23-58H/B1-182L × A19-61 CL-CH1) | 0.2363 | 0.6303 |
| MX468 (A19-61 × A23-58H/B1-182L CL-CH1) | 0.0848 | 0.1770 |
| MX179 (A19-46 × B1-182) | >66.7 | >66.7 |
| MX180 (B1-182 × A19-46) | >66.7 | >66.7 |
| MX494 (A19-61 × B1-182/A19-46 × B1-182) | 1.0060 | 1.6736 |
| MX495 (A19-61 × B1-182/B1-182 × A19-46) | 0.9952 | 2.1992 |
| MX496 (B1-182 × A19-61/A19-46 × B1-182) | 3.1340 | 9.3243 |
| MX497 (B1-182 × A19-61/B1-182 × A19-46) | 2.6723 | 6.1168 |
| MX490 (A23-58H/B1-182L × A19-46) | >6.7 | >6.7 |
| MX491 (A23-58H/B1-182L × A19-46 CL-CH1) | >66.7 | >66.7 |
| MX492 (A19-46 × A23-58H/B1-182L) | >66.7 | >66.7 |
| MX493 (A19-46 × A23-58H/B1-182L CL-CH1) | >66.7 | >66.7 |
| MX498 (A23-58H/B1-182L × A19-46/A23-58H/ | 11.2154 | 30.9541 |
| B1-182L × A19-61) | | |
| MX499 (A23-58H/B1-182L × A19-46/A19-61 × | 3.3621 | 8.4065 |
| A23-58H/B1-182L) | | |
| MX500 (A19-46 × A23-58H/B1-182L/A23-58H/ | 8.0925 | 16.3658 |
| B1-182L × A19-61) | | |
| MX501 (A19-46 × A23-58H/B1-182L/A19-61 × | 1.8386 | 4.3657 |
| A23-58H/B1-182L) | | |

These results show that the bispecific, tetravalent antibodies inhibited the Omicron variants much more potently than the parental antibodies.

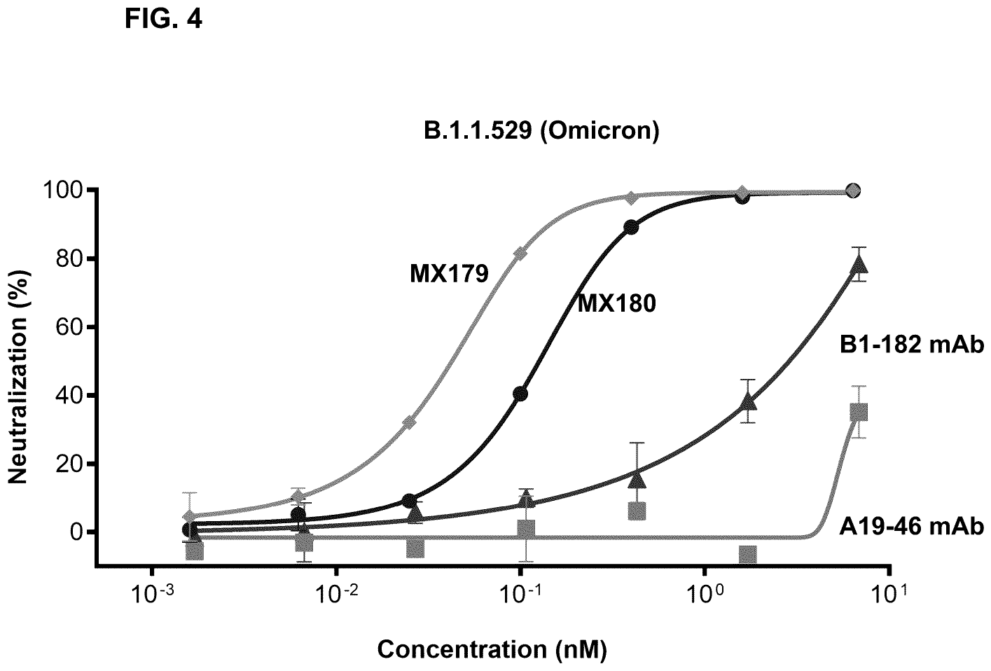

FIG. 4 shows the percent neutralization of Omicron variant (B.1.1.529) in the presence of increasing concentration of MX179 or MX180 antibody described in Example 1, along with B1-182 and A19-46 parental antibodies. The bispecific, tetravalent antibodies inhibited the Omicron variant much more potently than the parental antibodies.

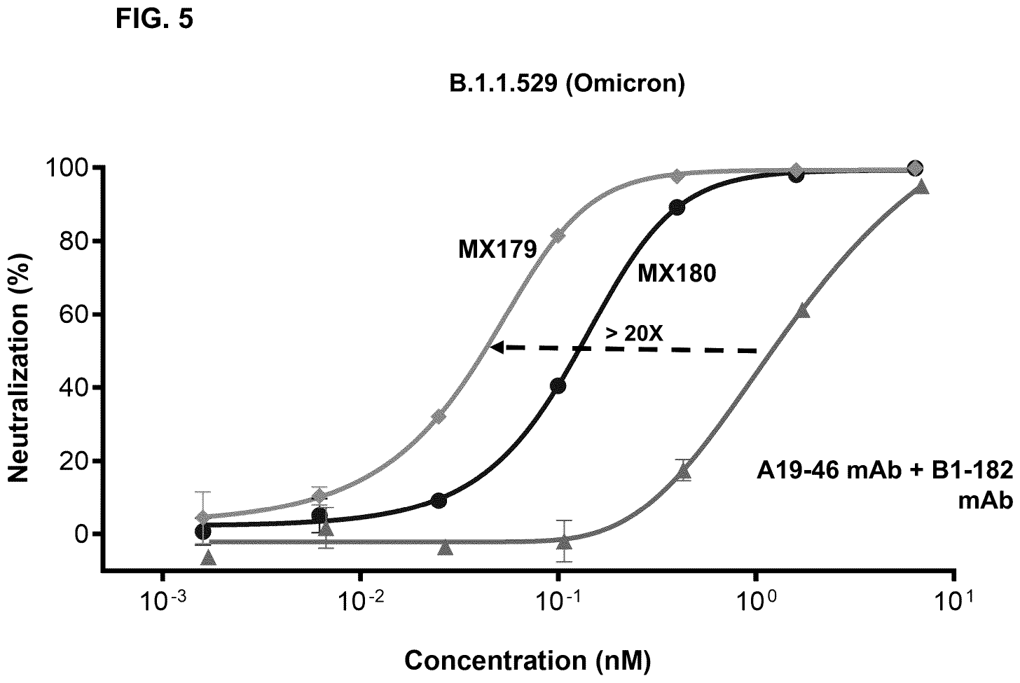

FIG. 5 shows the percent neutralization of Omicron variant (B.1.1.529) in the presence of increasing concentration of MX179 or MX180 antibody described in Example 1, in comparison to the combination of B1-182 and A19-46 parental antibodies. The bispecific, tetravalent antibodies inhibited the Omicron variant much more potently than the combination of parental antibodies, showing a synergistic effect of the multivalency. Concentration of each antibody in the combination of three antibody mixture was plotted.

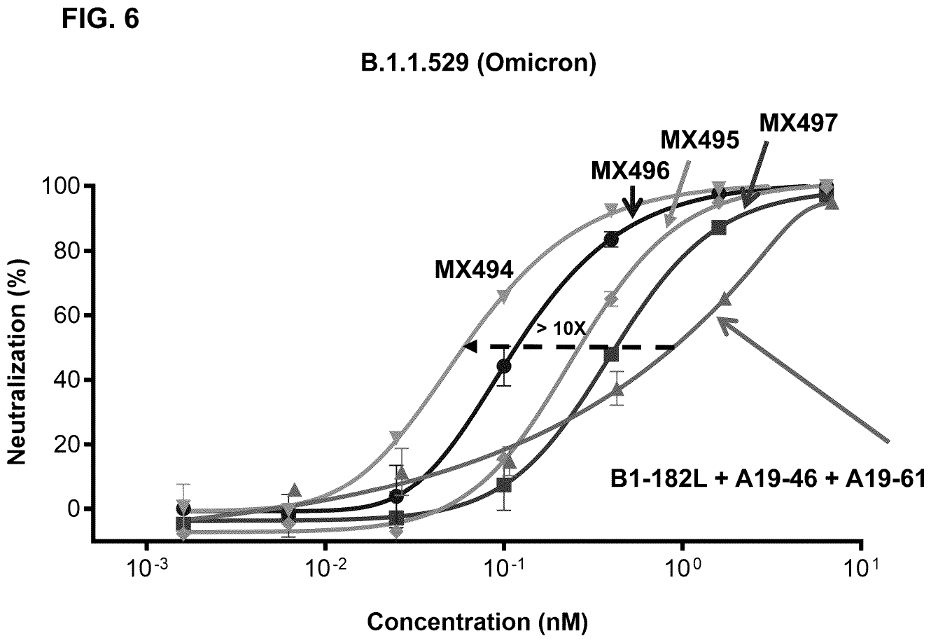

FIG. 6 shows the percent neutralization of Omicron variant (B.1.1.529) in the presence of increasing concentration of MX494, MX495, MX496 or MX497 antibody described in Example 1, in comparison to the combination of B1-182L, A19-46 and A19-61 parental antibodies. The trispecific, tetravalent antibodies inhibited the Omicron variant much more potently than the combination of parental antibodies, showing a synergistic effect of the multivalency. Concentration of each antibody in the combination of three antibody mixture was plotted.

Table 8 shows the IC50 and IC80 values (in nM) of MX828, MX829, MX830 and MX831 tetravalent, bispecific antibodies for neutralization of the SARS-CoV-2 variants, Delta (B.1.617.2) and Omicron (B.1.1.529, BA.2, BA.2.12.1, BA.4/5 and BA.1.1 (R346K)). IC50 and IC80 values for MX492, MX413, bebtelovimab (LY-CoV1404), S2E12, A23-58.1HC/58.1LC_93VGLTG, A23-58HC/58.1LC_94GLTG, and B1-182.1 are also shown for comparison. The bispecific, tetravalent antibodies potently inhibited the SARS-CoV-2 variants.

TABLE 8

| | | | Delta | | Omicron | | Omicron Sublineages | |
|---|---|---|---|---|---|---|---|---|
| | | | B.1.617.2 | | B.1.1.529 | | BA.2 | |
| Name | Antibody | Class | IC50 | IC80 | IC50 | IC80 | IC50 | IC80 |
| Bi-specific | MX828 | I + II | 74.83 | 172.86 | 4.3 | 19.48 | 13.73 | 20.27 |
| | MX829 | I + III | 29.55 | 97.83 | 34.81 | 70.13 | 3.58 | 6.06 |
| | MX830 | I + II | 63.74 | 125.32 | 10.98 | 23.46 | 9.41 | 10.75 |
| | MX831 | I + III | 29.2 | 84.01 | 75.74 | 131.63 | 0.03 | 0.08 |
| | MX492 | I + II | 41.59 | 97.83 | 9.42 | 15.89 | 15.15 | 31.18 |
| | MX413 | I + III | 29.53 | 101.22 | 156.36 | 3954.83 | 7.69 | 25.07 |
| bebtelovimab | LY-CoV1404 | III | 8.18 | 9.06 | 0.32 | 15.58 | 14.3 | 18.48 |
| | A23-58.1 | I | 1.4 | 4.7 | 98.1 | 781.8 | 262.7 | 1076.2 |
| | S2E12 | I | 1.2 | 2.6 | 8.8 | 38.5 | 9.9 | 48.2 |
| | A23-58.1HC/58.1LC_93VGLTG | I | 5.8 | 12.4 | 3.3 | 11.2 | n.t. | n.t. |
| | A23-58.1HC/58.1LC_94GLTG | I | 2.9 | 11.2 | 3.6 | 11.9 | n.t. | n.t. |
| | B1-182.1 | I | 1.57 | 7.25 | 400.98 | 1015.63 | 319.5 | 1212.04 |

| | | Omicron Sublineages | | | | | |
|---|---|---|---|---|---|---|---|
| | | | | | | BA.1.1 | |
| | | BA.2.12.1 | | BA.4/5 | | (R346K) | |
| Name | Antibody | IC50 | IC80 | IC50 | IC80 | IC50 | IC80 |
| Bi-specific | MX828 | 43.02 | 74.97 | 1007 | 5323.87 | 1.75 | 6.23 |
| | MX829 | 3.96 | 8.4 | 11.48 | 34.71 | 6.72 | 35.4 |
| | MX830 | 62.71 | 94.29 | 532.86 | 4628.36 | 10.39 | 11.49 |
| | MX831 | 38.03 | 42.94 | 25.32 | 47.02 | 17.25 | 50 |
| | MX492 | 62.91 | 180.86 | >10,000 | >10,000 | 8.77 | 67.97 |
| | MX413 | 6.32 | 32.12 | 51.24 | 73.92 | 2.24 | 10.48 |
| bebtelovimab | LY-CoV1404 | 8.52 | 14.82 | 1.88 | 3.74 | 26.44 | 65.08 |
| | A23-58.1 | 100.1 | 503.6 | >10,000 | >10,000 | n.t. | n.t. |
| | S2E12 | 15.6 | 46.6 | 713.1 | 3971.9 | n.t. | n.t. |
| | A23-58.1HC/58.1LC_93VGLTG | 3.4 | 11.5 | 3 | 13.7 | n.t. | n.t. |
| | A23-58.1HC/58.1LC_94GLTG | 1.9 | 9.2 | 4.7 | 29.6 | n.t. | n.t. |
| | B1-182.1 | 165.48 | 1375.63 | >10,000 | >10,000 | 213.36 | 1297.92 |

Table 9 shows the IC50 and IC80 values (in nM) of MX828, MX829, MX830 and MX831 tetravalent, bispecific antibodies for neutralization of the SARS-CoV-2 variants, Omicron BA.1.1 (R346K), BA.4.6 (R346T, F486V), BA.4.7 (R346S, F486V), BF.7 (R346T, F486V), BA.2.75.2 (R346T, F486S), and BQ.1.1 (R346T, K444T, F486V). MX492 and MX413 and bebtelovimab (LY1404) were also tested for comparison. And, in addition, combinations of MX828 and MX829 (MX828+MX829), MX830 and MX831 (MX830+MX831) and MX492 and MX413 (MX492+MX413) were also tested. The bispecific, tetravalent antibodies potently inhibited the SARS-CoV-2 variants.

TABLE 9

| | | | Omicron Sublineages | | | | | | |
|---|---|---|---|---|---|---|---|---|---|
| | | | BA.1.1 | | BA.4.6 | | BA.4.7 | | BF.7 |
| | | | (R346K) | | (R346T, F486V) | | (R346S, F486V) | | (R346T, F486V) |
| Name | Antibody | Class | IC50 | IC80 | IC50 | IC80 | IC50 | IC80 | IC50 |
| Bi-specific | MX828 | I + II | 1.75 | 6.23 | 123.76 | 1642.22 | 143.42 | 1756.83 | 356.39 |
| | MX829 | I + III | 6.72 | 35.4 | 17.16 | 42.7 | 2.37 | 12.96 | 3.45 |
| | MX828 + MX829 | I + II + III | n.t. | n.t. | 15.04 | 40.36 | 10.04 | 31.13 | 5.26 |
| | MX830 | I + II | 10.39 | 11.49 | 207.08 | 4648.77 | 275.85 | >10,000 | 859.58 |
| | MX831 | I + III | 17.25 | 50 | 27.91 | 58.48 | 3.08 | 19.12 | 3.73 |
| | MX830 + MX831 | I + II + III | n.t. | n.t. | 21.51 | 45.42 | 8.45 | 30.62 | 3.91 |
| | MX492 | I + II | 8.77 | 67.97 | 21.4 | 24.51 | 13.35 | 154.17 | >10,000 |
| | MX413 | I + III | 2.24 | 10.48 | 55.95 | 154.8 | 77.93 | 126.15 | 28.15 |
| | MX492 + MX413 | I + II + III | n.t. | n.t. | 94.04 | 279.93 | 22.37 | 126.81 | 131.91 |
| bebtelovimab | LY1404 | III | 26.44 | 65.08 | 22.33 | 25.43 | 5.9 | 16.05 | 16.92 |

| | | Omicron Sublineages | | | | |
|---|---|---|---|---|---|---|
| | | BF.7 | BA.2.75.2 | | BQ 1.1 | |
| | | (R346T, F486V) | (R346T, F486S) | | (R346T, K444T, F486V) | |
| Name | Antibody | IC80 | IC50 | IC80 | IC50 | IC80 |
| Bi-specific | MX828 | 1423.99 | 2464.9 | 7944.2 | 374.76 | 1125.06 |
| | MX829 | 22.17 | >10,000 | >10,000 | 1923.25 | 8867.36 |
| | MX828 + MX829 | 22.45 | 8521.96 | >10,000 | 537.73 | 1968.78 |
| | MX830 | 4381.87 | 7054.01 | >10,000 | 629.41 | 2709.82 |
| | MX831 | 21.68 | >10,000 | >10,000 | 4213.88 | >10,000 |
| | MX830 + MX831 | 24.82 | 3709.91 | 9721.44 | 421.55 | 3821.38 |
| | MX492 | >10,000 | 6063.34 | >10,000 | >10,000 | >10,000 |
| | MX413 | 177.41 | >10,000 | >10,000 | >10,000 | >10,000 |
| | MX492 + MX413 | 253.87 | 2482.17 | 5527.97 | >10,000 | >10,000 |
| bebtelovimab | LY1404 | 25.2 | 4.98 | 20.22 | >10,000 | >10,000 |

Table 10 shows the IC50 and IC80 values (in nM) of MX834, MX837, MX835 and MX838 tetravalent, trispecific antibodies for neutralization of the SARS-CoV-2 variants, Delta (B.1.617.2) and Omicron (B.1.1.529, BA.2, BA.2.12.1, BA.4/5 and BA.1.1 (R346K)). IC50 and IC80 values for MX500 and MX501 are also shown for comparison. The tetravalent, trispecific antibodies potently inhibited the SARS-CoV-2 variants.

TABLE 10

| | | | Delta | | Omicron | | Omicron Sublineages | | | | | | | |
|---|---|---|---|---|---|---|---|---|---|---|---|---|---|---|
| | | | B.1.617.2 | | B.1.1.529 | | BA.2 | | BA.2.12.1 | | BA.4/5 | | BA.1.1 (R346K) | |
| Name | Antibody | Class | IC50 | IC80 | IC50 | IC80 | IC50 | IC80 | IC50 | IC80 | IC50 | IC80 | IC50 | IC80 |
| Tri-specific | MX834 | I + II + III | 7.32 | 11.26 | 1.4 | 6.58 | 3.17 | 26.03 | 4.62 | 12.06 | 12.5 | 29.95 | 1.6 | 3.92 |
| | MX837 | I + II + III | 10.29 | 11.26 | 2.23 | 4.45 | 2.06 | 7.07 | 5.76 | 17.54 | 5.93 | 17.45 | 9.72 | 11.4 |
| | MX835 | I + II + III | 7.96 | 34.94 | 1.43 | 4.85 | 3.22 | 17.09 | 3.06 | 13.38 | 8.32 | 27.35 | 6.52 | 11.71 |
| | MX838 | I + II + III | 36.41 | 39.93 | 4.3 | 9.1 | 3.82 | 23.84 | 6.01 | 18.4 | 7.84 | 16.02 | 1.17 | 3.23 |
| | MX500 | I + II + III | 4.53 | 10.72 | 6.07 | 30.83 | 1.59 | 10.09 | 16.57 | 84.93 | 1034.11 | 2370.06 | 45.11 | 83.38 |
| | MX501 | I + II + III | 4.82 | 5.44 | 7.19 | 23.15 | 1.66 | 11.67 | 17.12 | 51.02 | 85.24 | 606.61 | 20.49 | 77.18 |

Table 11 shows the IC50 and IC80 values (in nM) of MX834, MX837, MX835 and MX838 tetravalent, trispecific antibodies for neutralization of the SARS-CoV-2 variants, Omicron BA.1.1 (R346K), BA.4.6 (R346T, F486V), BA.4.7 (R346S, F486V), BF.7 (R346T, F486V), BA.2.75.2 (R346T, F486S), and BQ.1.1 (R346T, K444T, F486V). MX500, MX501 and bebtelovimab (LY1404) were also tested for comparison. The tetravalent, trispecific antibodies potently inhibited the SARS-CoV-2 variants.

TABLE 11

| | | | Omicron Sublineages | | | | | | |
|---|---|---|---|---|---|---|---|---|---|
| | | | BA.1.1 | | BA.4.6 | | BA.4.7 | | BF.7 |
| | | | (R346K) | | (R346T, F486V) | | (R346S, F486V) | | (R346T, F486V) |
| Name | Antibody | Class | IC50 | IC80 | IC50 | IC80 | IC50 | IC80 | IC50 |
| Tri-specific | MX834 | I + II + III | 1.6 | 3.92 | 68.44 | 174.43 | 39.67 | 186.99 | 65.91 |
| | MX837 | I + II + III | 9.72 | 11.4 | 32.92 | 95.5 | 4.19 | 27.91 | 40.46 |
| | MX835 | I + II + III | 6.52 | 11.71 | 71.14 | 178.67 | 40.95 | 216.03 | 61.42 |
| | MX838 | I + II + III | 1.17 | 3.23 | 38.83 | 115.19 | 11.69 | 46.49 | 27.1 |
| | MX500 | I + II + III | 45.11 | 83.38 | >10,000 | >10,000 | >10,000 | >10,000 | >10,000 |
| | MX501 | I + II + III | 20.49 | 77.18 | >10,000 | >10,000 | >10,000 | >10,000 | >10,000 |
| bebtelovimab | LY1404 | III | 26.44 | 65.08 | 11.5 | 28.15 | 11.44 | 22.45 | 8.51 |

| | | Omicron Sublineages | | | | |
|---|---|---|---|---|---|---|
| | | BF.7 | BA.2.75.2 | | BQ 1.1 | |
| | | (R346T, F486V) | (R346T, F486S) | | (R346T, K444T, F486V) | |
| Name | Antibody | IC80 | IC50 | IC80 | IC50 | IC80 |
| Tri-specific | MX834 | 244.45 | >10,000 | >10,000 | 5122.91 | >10,000 |
| | MX837 | 125.47 | >10,000 | >10,000 | 4108.69 | 4625.22 |
| | MX835 | 281.82 | >10,000 | >10,000 | 4243.71 | >10,000 |
| | MX838 | 132.16 | >10,000 | >10,000 | 5832.55 | >10,000 |
| | MX500 | >10,000 | >10,000 | >10,000 | >10,000 | >10,000 |
| | MX501 | >10,000 | >10,000 | >10,000 | >10,000 | >10,000 |
| bebtelovimab | LY1404 | 24.54 | 5.67 | 20.72 | >10,000 | >10,000 |

Table 12 shows the IC50 and IC80 values (in nM) of MX834, MX835, MX837 and MX838 tetravalent, trispecific antibodies, and MX828 and MX829 tetravalent, bispecific antibodies for neutralization of the SARS-CoV-2 variants, D614G, BA. 1, BA.4/5 (F486V), BA.4.6 (R346T, F486V), BA.2.75 (G446S), BA.2.75.2 (R346T, G446S, F486S), BQ.1.1 (R346T, K44T, F486V) and BJ.1 (R346T, V445P, G446S). MX500, MX501, MX492, MX413, B10, B11, 58H/182L, 58.1LC_94GLTG, 58.1LC_93VGLTG, 46.1, 61.1 and bebtelovimab (LY1404) were also tested for comparison. In addition, combinations of MX828 and MX829 (MX828+829), MX830 and MX831 (MX830+831), and MX492 and MX413 (MX492+413) were also tested. The tetravalent, trispecific and tetravalent, bispecific antibodies potently inhibited the SARS-CoV-2 variants.

TABLE 12

| | | | | Omicron Sublineages | | | | | |
|---|---|---|---|---|---|---|---|---|---|
| | | | | | | BA.4/5 | | BA.4.6 | |
| | | D614G | | BA.1 | | (F486V) | | (R346T, F486V) | |
| ID | Class | IC50 | IC80 | IC50 | IC80 | IC50 | IC80 | IC50 | IC80 |
| MX834 | I + II + III | 1 | 8 | 2 | 8 | 9 | 35 | 24 | 109 |
| MX835 | I + II + III | 2 | 8 | 2 | 8 | 8 | 30 | 19 | 92 |
| MX837 | I + II + III | 2 | 9 | 2 | 7 | 9 | 17 | 33 | 80 |
| MX838 | I + II + III | 3 | 14 | 1 | 4 | 9 | 18 | 23 | 104 |
| MX500 | I + II + III | 0 | 2 | 5 | 24 | 400 | 2583 | 3440 | >10,000 |
| MX501 | I + II + III | 1 | 5 | 2 | 11 | 203 | 1202 | 2017 | 3120 |
| MX828 | I + II | 4 | 9 | 1 | 3 | 86 | 438 | 54 | 300 |
| MX829 | I + III | 3 | 5 | 14 | 43 | 2 | 4 | 4 | 13 |
| MX828 + 829 | I + II + III | 3 | 4 | 2 | 4 | 2 | 6 | 5 | 10 |
| MX830 | I + II | 5 | 9 | 1 | 3 | 260 | 1565 | 138 | 514 |
| MX831 | I + III | 3 | 5 | 7 | 25 | 2 | 4 | 3 | 10 |
| MX830 + 831 | I + II + III | 2 | 4 | 2 | 3 | 2 | 4 | 4 | 12 |
| MX492 | I + II | 5 | 8 | 1 | 3 | >10,000 | >10,000 | >10,000 | >10,000 |
| MX413 | I + III | 3 | 4 | 1294 | 6545 | 4 | 8 | 10 | 25 |
| MX492 + 413 | I + II + III | 2 | 3 | 2 | 4 | 4 | 11 | 12 | 31 |
| B10 | I + III | 3 | 11 | 3 | 12 | 5 | 16 | 9 | 83 |
| B11 | I + III | 4 | 12 | 5 | 21 | 6 | 17 | 14 | 91 |
| 58H/182L | I | >10,000 | >10,000 | 53 | 90 | >10,000 | >10,000 | >10,000 | >10,000 |
| 58.1LC_94GLTG | I | 0 | 3 | 2 | 8 | 5 | 20 | 2 | 10 |
| 58.1LC_93VGLTG | I | 0 | 2 | 1 | 9 | 4 | 21 | 1 | 9 |
| 46.1 | II | >10,000 | >10,000 | 77 | 211 | >10,000 | >10,000 | >10,000 | >10,000 |
| 61.1 | III | >10,000 | >10,000 | >10,000 | >10,000 | 19 | 31 | 540 | 2170 |
| LY1404 | III | 0 | 1 | 1 | 7 | 0 | 1 | 0 | 1 |

| | Omicron Sublineages | | | | | | | |
|---|---|---|---|---|---|---|---|---|
| | BA.2.75 | | BA.2.75.2 | | BQ.1.1 | | BJ.1 | |
| | (G446S) | | (R346T, G446S, F486S) | | (R346T, K444T, F486V) | | (R346T, V445P, G446S) | |
| ID | IC50 | IC80 | IC50 | IC80 | IC50 | IC80 | IC50 | IC80 |
| MX834 | 1 | 2 | >10,000 | >10,000 | 1001 | 4056 | 3 | 87 |
| MX835 | 1 | 2 | >10,000 | >10,000 | 369 | 1624 | 2 | 23 |
| MX837 | 1 | 3 | >10,000 | >10,000 | 1444 | 5629 | 15 | 97 |
| MX838 | 1 | 3 | >10,000 | >10,000 | 2375 | 7000 | 18 | 85 |
| MX500 | 1 | 2 | 4127 | >10,000 | >10,000 | >10,000 | 172 | 2641 |
| MX501 | 1 | 7 | 6716 | >10,000 | >10,000 | >10,000 | 421 | 2004 |
| MX828 | 1 | 4 | 781 | 2757 | 164.38 | 663 | 7 | 37 |
| MX829 | 1 | 3 | >10,000 | >10,000 | 1115 | 2591 | 16 | 113 |
| MX828 + 829 | 0 | 1 | 1378 | 3219 | 91 | 588 | 5 | 20 |
| MX830 | 1 | 4 | 1261 | 5887 | 359 | 1300 | 3 | 24 |
| MX831 | 1 | 2 | >10,000 | >10,000 | 1485 | 6704 | 40 | 169 |
| MX830 + 831 | 1 | 2 | 2157 | 6030 | 261 | 878 | 6 | 29 |
| MX492 | 3 | 10 | 1627 | 6735 | >10,000 | >10,000 | 783 | 2011 |
| MX413 | 21 | 71 | >10,000 | >10,000 | >10,000 | >10,000 | 3208 | >10,000 |
| MX492 + 413 | 3 | 6 | 2094 | 4558 | >10,000 | >10,000 | 718.9 | 3207 |
| B10 | 1 | 2 | >10,000 | >10,000 | 206 | 603 | 12 | 39 |
| B11 | 1 | 2 | >10,000 | >10,000 | 342 | 1534 | 3 | 41 |
| 58H/182L | 5 | 35 | >10,000 | >10,000 | >10,000 | >10,000 | 20 | 102 |
| 58.1LC_94GLTG | 0 | 1 | >10,000 | >10,000 | 12 | 38 | 1 | 7 |
| 58.1LC_93VGLTG | 0 | 1 | >10,000 | >10,000 | 7 | 28 | 0 | 2 |
| 46.1 | 148 | 352 | 386 | 1111 | >10,000 | >10,000 | >10,000 | >10,000 |
| 61.1 | >10,000 | >10,000 | >10,000 | >10,000 | >10,000 | >10,000 | >10,000 | >10,000 |
| LY1404 | 13 | 86 | 2 | 7 | >10,000 | >10,000 | >10,000 | >10,000 |

### Example 3

### Half-Life Analysis

The half-life of tetravalent bispecific antibody MX413 (A19-61×A23-58H/1B1-182L, containing a Fc mutation to extend half-life) was determined using a mouse model. Tg32-hFc mice (B6.Cg-Tg(FCGRT)32Dcr FcgrttmlDer Ighglem2(IGHG1)Mvw/MvwJ, strain #029686) between the ages of 6-12 weeks were obtained from The Jackson Laboratory (Bar Harbor, ME). On Day 0, all mice were treated with intravenous (IV) injection of MX413, parental B1-182.1 or VRC01LS antibody at a dose of 5 mg/kg. In-life blood and serum collections in ethylenediaminetetraacetic acid (EDTA) were conducted on Days −7, 0, 1, 2, 5, 7, 11, 14, 21, 28, 35, 42, and 49. All mice had maximum bleeds conducted on Day 56 and were then euthanized. Antibody concentration in serum was measured by quantitative ELISA using SARS-CoV-2 spike protein-coated microtiter plates and a horseradish peroxidase (IRP)-conjugated anti-human IgG antibody.

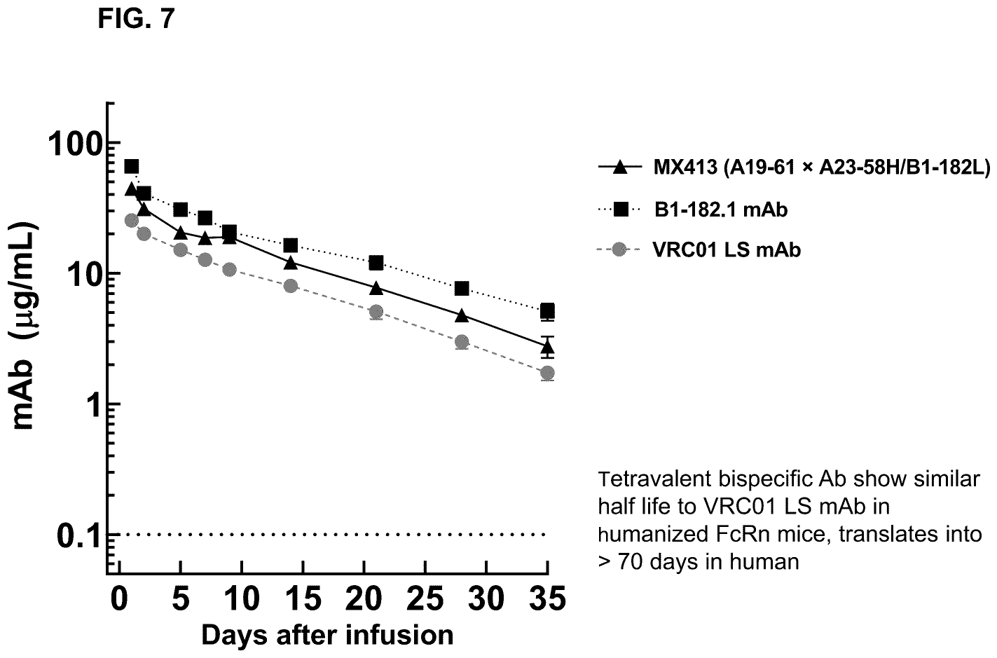

FIG. 7 shows serum antibody levels following treatment. These data show that MX413 had a similar half-life to VRC01 LS, representing a half-life of greater than 70 days in human.

### Example 4

### Tetravalent Trispecific Antibody Protects Syrian Hamsters from SARS-CoV-2 Variants

The tetravalent trispecific antibody, 46.1-182.1v/61.1-182.1v (MX501; SEQ ID NOs:112, 113, 148 and 149) was tested in an in vivo passive challenge study to determine its capacity to protect Syrian golden hamsters from infection by Omicron variants BA.1, BA.2 or BA.5. MX501 contains single Fv46 and Fv61 domains and two Fv182v domains (FIG. 3H). Fv182v is a stabilized variant of Fv182. MX501 showed similar breadth and potency to 61.1/46.1-182.1 (LY-CoV555), as shown below in Table 13.

TABLE 13
MX501 (46.1-182.1v/61.1-182.1v) is a Potently Neutralizing Tetravalent Trispecific Antibody

| | B.1.1.7 | | BA.1 | | BA.2 | | BA.4/5 | |
|---|---|---|---|---|---|---|---|---|
| | IC50 | IC80 | IC50 | IC80 | IC50 | IC80 | IC50 | IC80 |
| LY-CoV555 | 14.5 | 36.1 | >67,000 | >67,000 | >67,000 | >67,000 | >67,000 | >67,000 |
| 46.1-182.1v/61.1-182.1v | 18.7 | 71.0 | 28.5 | 87.6 | 9.7 | 25.0 | 1839 | 4366 |

Neutralization is shown in pM.

For the passive challenge study, all experiments were conducted according to the National Institutes of Health (NIH) regulations and standards on the humane care and use of laboratory animals, as well as the Animal Care and Use Committees of Bioqual, Inc. (Rockville, Maryland). Six- to eight-week-old male Syrian hamsters (Envigo) were housed at Bioqual, Inc. Multispecific antibody was diluted into phosphate buffered saline (PBS) to achieve a dose of 10 mg/kg in a 1-2 mL volume and passively transferred to the hamsters by injection into the peritoneal cavity 24 hours prior to challenge. Hamsters were infected with SARS-CoV-2 variants by injecting 100 μL of virus diluted in PBS into the nares, split between both nostrils. Weight changes and clinical observations were collected daily for all studies. Four hamsters per group were euthanized at days 2, 4, 6 and 10 to collect lungs for quantification of viral load.

The BA.1 SARS-CoV-2 variant used in this study was described in Gagne, M. et al., Cell 185, 1556-1571.e18 (2022) and Gagne, M. et al., Cell 185, 113-130.e15 (2022). The BA.2 SARS-CoV-2 variant was obtained from the Biodefense and Emerging Infections Research Resources Repository (BEI, NR-56522). The BA.5 SARS-CoV-2 variant was propagated by the laboratory of Mehul S. Suthar in Vero-TMPRSS2 cells. Sequence was confirmed prior to challenge studies. All SARS-CoV-2 stocks were titrated in hamsters to confirm pathogenicity prior to use.

For TCID50 quantification of SARS-CoV-2, tissues were weighed, placed into pre-labeled Sarstedt cryovials, and snap-frozen until needed. Prior to testing, tissues were thawed and homogenized using a hand-held tissue homogenizer. Samples were spun down to remove debris and supernatants were assayed. TCID50 was quantified by incubating Vero-TMPRSS2 cells at 37° C., 5% CO₂ overnight. The following day, the medium was aspirated and replaced with fresh medium. Samples were serially diluted ten-fold for quantification. Positive (virus stock of known infectious titer in the assay) and negative (medium only) control wells were included in every assay. The plates were incubated at 37° C., 5.0% CO₂ for 4 days. The cell monolayers were visually inspected for cytopathic effect. TCID50 values were calculated using the Reed-Muench formula. TCID50 values were log transformed prior to statistical analysis.

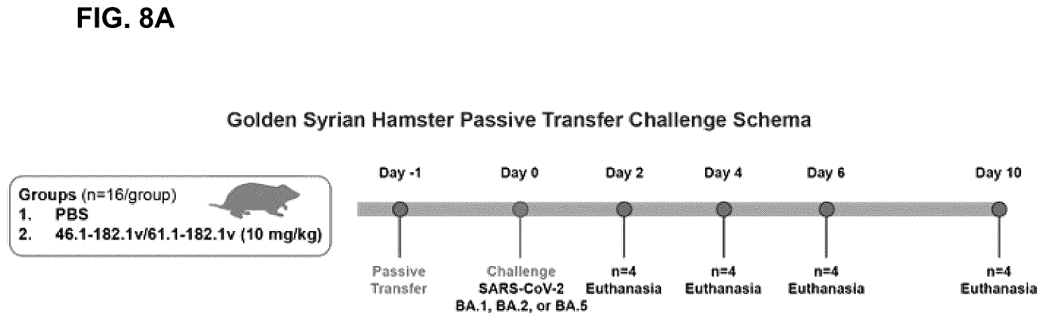

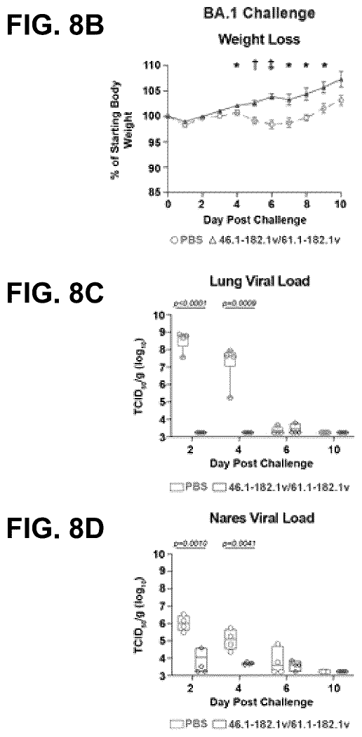

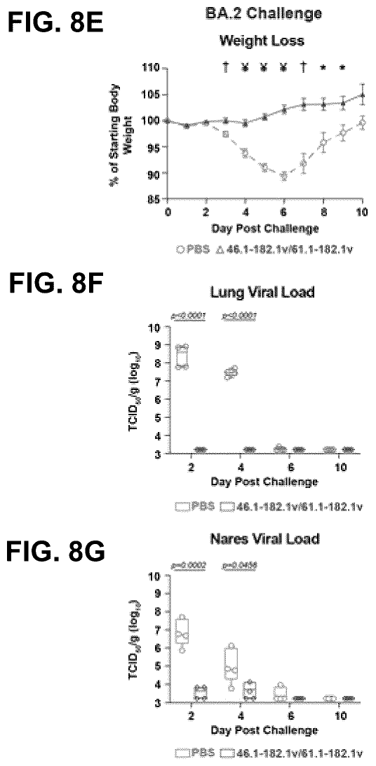

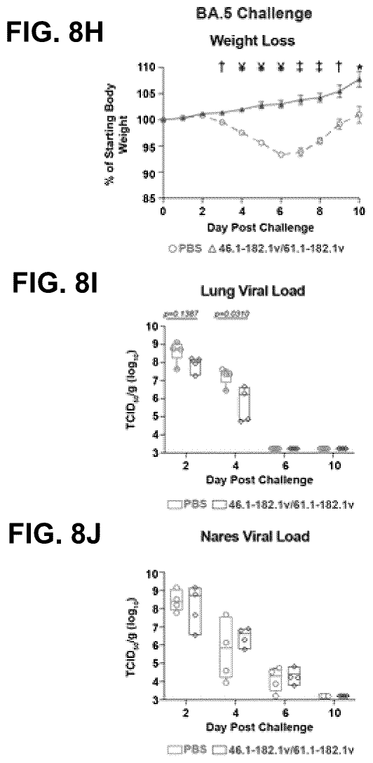

FIG. 8A shows a diagram of the passive transfer challenge study. Specifically, MX501 (46.1-182.1v/61.1-182.1v) was passively transferred to Syrian hamsters intraperitoneally 24 hours prior to challenge with either 1×10⁵ plaque-forming units (PFU) of BA.1, 2×10⁴ PFU of BA.2, or 1×10⁵ PFU of BA.5 SARS-CoV-2 Omicron variants. For each virus, an additional group of hamsters received an equal volume of PBS in the same manner and served as controls. All animals were weighed daily to monitor weight loss and 4 animals per group were euthanized at days 2, 4, 6, and 10 to assess viral loads in the lung and nares. Hamsters were monitored daily for weight loss over 10 days, and viral load in the lung and nares was also quantified by TCID50 per gram.

For BA.1-infected hamsters, MX501-treated animals continued to gain weight throughout the study, whereas PBS-treated animals lost weight from days 5-7 before recovering and regaining weight (FIG. 8B). Also, while PBS treated animals show >7-logs of lung viral load per gram (TCID50/g) up until day 4, antibody-treated animals had very low to undetectable lung viral load in the assay (FIG. 8C). For BA.2- and BA.5-infected hamsters, a 7-10% weight loss was observed on day 6 in PBS-treated animals, and an absence of weight loss in animals that received antibody (FIGS. 8E and 8H). Similar to BA.1, the viral titer for BA.2 was >7-logs of lung viral load per gram on days 2 and 4 in PBS-treated animals and no virus growth was observed in the lungs of treated animals (FIG. 8F). For BA.5, viral titer exceeded 8-logs on day 2 and antibody-treated animals showed a significant 13-fold lower lung viral load on day 4 relative to PBS treated animals (FIG. 8I).

In addition, while lung infection and weight loss are relevant for systemic disease, clearance of virus from the nares may be correlated with decreases in virus transmission. Viral loads in the nares of PBS-treated animals noted a peak average viral load 1.4×10⁶ per gram of tissue (TCID50/g) that occurred on day 2 and that virus did not clear until after day 6 in half the animals (FIG. 8D). In antibody-treated animals, low viral loads were present in treated animals until day 6 (FIG. 8D). This is consistent with other studies that showed the clearance of virus from the nares is more difficult than the lungs.

In the nares, PBS-treated hamsters had a peak viral load average, 1.5×10⁷ TCID50/g, that occurred on day 2 of infection and was a log higher than BA.1 (FIG. 8G). Despite the higher peak viral load in untreated animals, the antibody-treated animals had low viral loads similar to BA.1 on days 2 and 4, and had no virus growth staring on day 6 (FIG. 8G). And, in the nares, the average peak viral load was 5.0×10⁸, occurred on day 2 and was 10 to 100-fold higher than that seen with BA.1- and BA.2-infected hamsters. BA.5 virus growth in the nares on average lasted longer than BA.1 and BA.2 (FIG. 8J), suggesting it was more difficult for untreated animals to clear BA.5 infection from the nares. In antibody-treated animals, no significant difference was noted between the antibody and PBS control group.

Together, these data show that MX501 (46.1-182.1v/61.1-182.1v) has breadth, potency and in vivo protective efficacy against lower respiratory tract disease caused by Omicron and Omicron sublineages with distinct amino acid substitutions that mediate escape from therapeutic antibodies.

### Example 5

### SARS-CoV-2 Escape Potential by Multispecific Antibodies

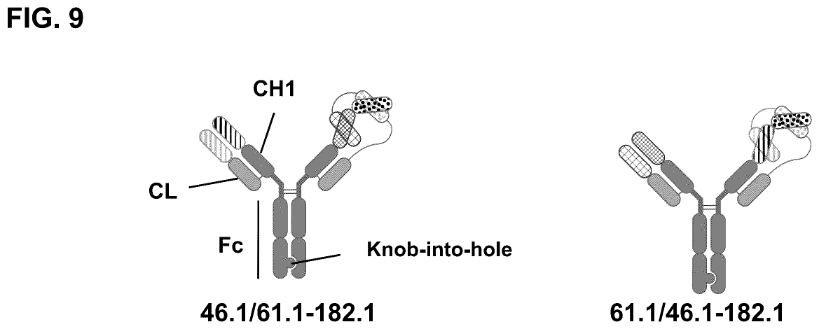

FIG. 9 shows two configurations of trispecific, trivalent cross-over dual variable (CODV) antibodies made utilizing variable fragments (Fv) from B1-182.1 (“182.1”, dotted; light chain=lighter shading; heavy chain=darker shading), the anti-SARS-CoV-2 antibody, A19-46.1 (“46.1”, striped; light chain=lighter shading; heavy chain=darker shading) and A19-61.1 (“61.1”, grid; light chain=lighter shading; heavy chain=darker shading). The two configurations, 46.1/61.1-182.1 and 61.1/46.1-182.1, correspond to SEQ ID NOs:150-157 and SEQ ID NOs:158-165, respectively.

After design of the amino acid sequences for each multispecific antibody, the four genes for each multispecific antibody were synthesized using human preferred codons (GenScript) and cloned into eukaryotic expression vectors. For each multispecific antibody expression, equal amounts of the 4 plasmid DNAs were transfected into Expi293 cells (Life Technology) using Expi293 transfection reagent (Life Technology) as previously reported (DOI: 10.1126/science.aan8630). The transfected cells were cultured in a shaker incubator at 120 rpm, 37° C., 9% CO₂ for 4-5 days.

Culture supernatants were harvested and filtered, the multispecific antibodies were purified over a Protein A (GE Health Science) column. Each multispecific antibody was eluted with IgG elution buffer (Pierce), immediately buffer exchanged with phosphate buffered saline (PBS) and concentrated using Centricon Plus-70 (Millipore Sigma) membrane filter unit. After concentration, each multispecific antibody was applied to a Superdex 200 16/600 size exclusion column (Cytiva) to remove aggregates and different species in the preparation. The fractions were then analyzed on reduced and non-reduced SDS-PAGE to identify the fractions that contained the monomeric multispecific antibody before combining them. The pooled fractions were then further concentrated, aliquoted and analyzed by SDS-PAGE as well as an analytical SEC column (Superdex 200 16/600) to verify purity.

The escape potential of the multispecific antibodies was assessed using an rcVSV antibody escape assay. Selection of virus escape variants was conducted as previously described by Wang et al., Science, 373:1-15 (2021). Briefly, replication competent VSV (rcVSV) bearing SARS-CoV-2 WA-1 spike protein were incubated with the indicated antibodies at 5-fold increasing concentrations (34×10⁻³ to 333,333 μM). Vero cells in 12-well plates were infected with virus-antibody mixture to allow viral replication given selective pressure from antibodies. Three days after infection, wells were assessed for significant viral growth, as indicated by the presence of >20% CPE. Supernatant from the well showing significant viral growth at the highest antibody concentration was passaged forward onto fresh cells and antibody containing media. Shown for each antibody is the maximum concentration with >20% CPE that was passaged forward in each round of selection. Once significant viral growth appeared at 333,333 μM, supernatant was no longer passaged forward, and generation of escape variants was considered fixed. Three independent experiments were carried out, and compared to mAb LY-COV1404.

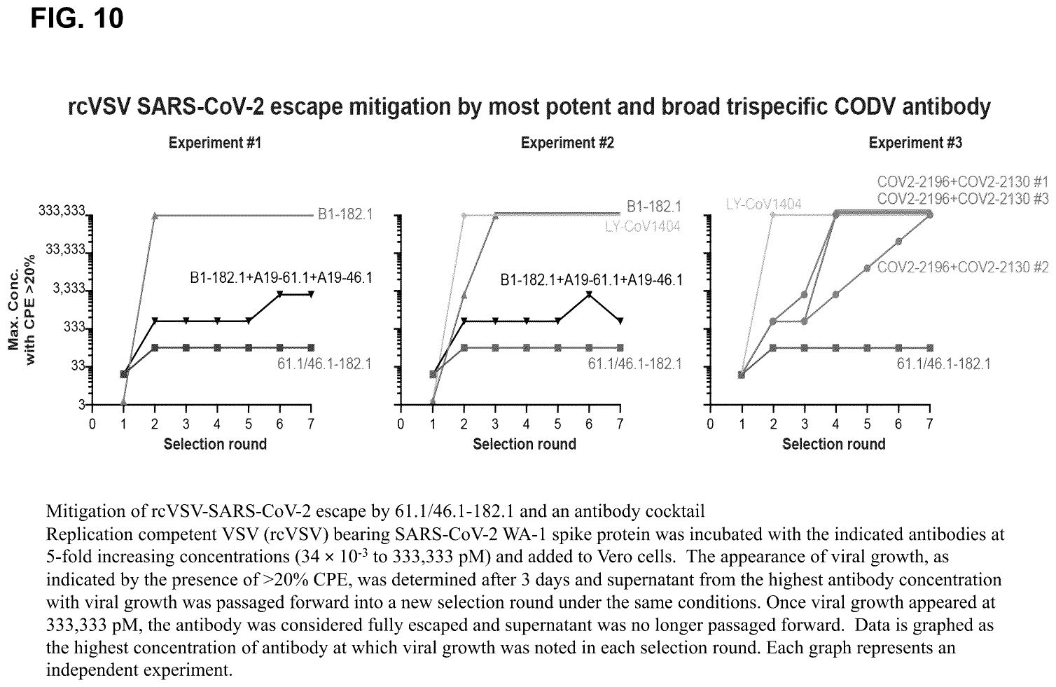

FIG. 10 shows SARS-CoV-2 escape potential by monospecific antibodies (B1-182.1), a combination of monospecific antibodies (B1-182.1+A19-61.1+A19-46.1), and multispecific antibodies (46.1/61.1-182.1 and 61.1/46.1-182.1). Shown for each antibody is the maximum concentration with >20% CPE that was passaged forward in each round of selection. Once significant viral growth appeared at 333,333 μM, supernatant was no longer passaged forward, and generation of escape variants was considered fixed.

These results show that the candidate multispecific antibodies mitigated immune escape observed with single antibodies and combinations of single antibodies.

### Example 6

### Pseudovirus Neutralization Assay

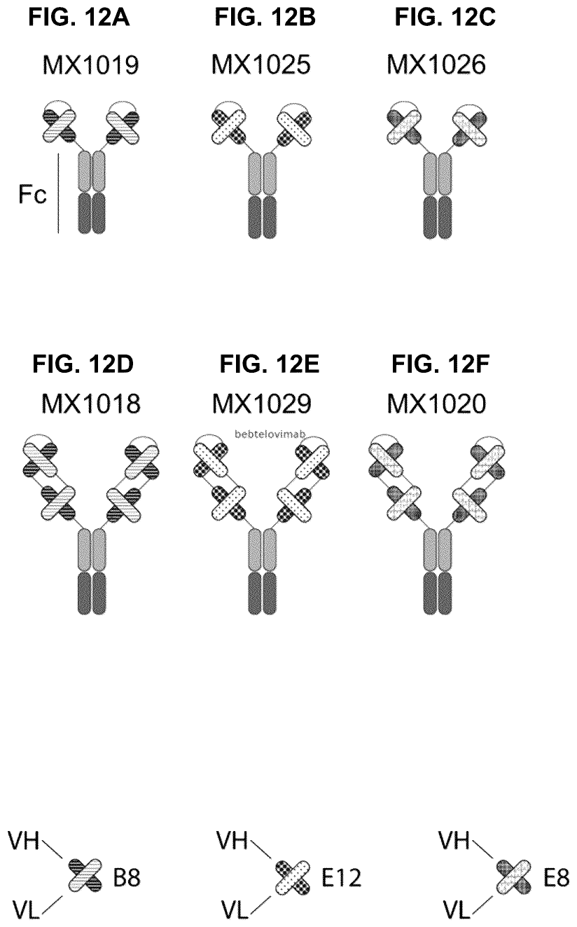

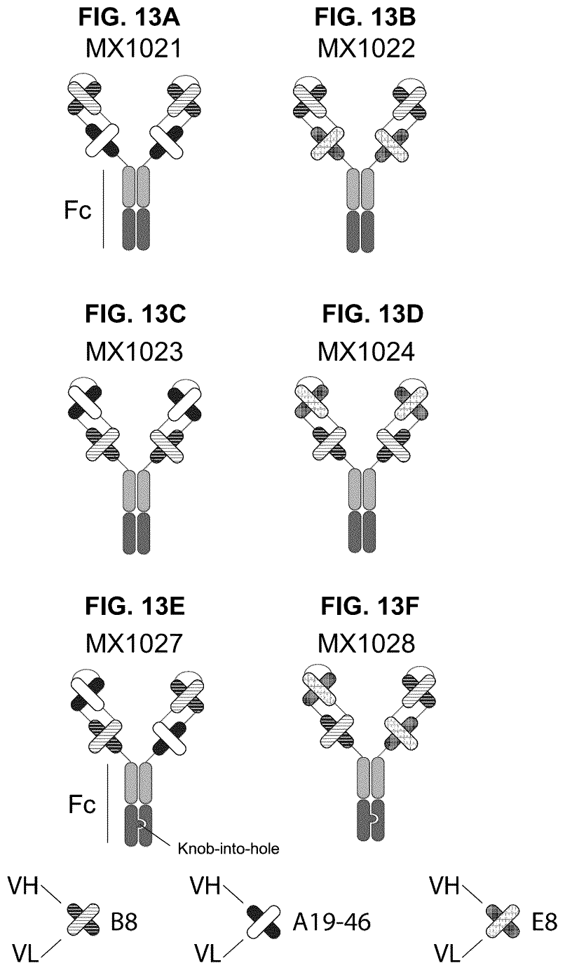

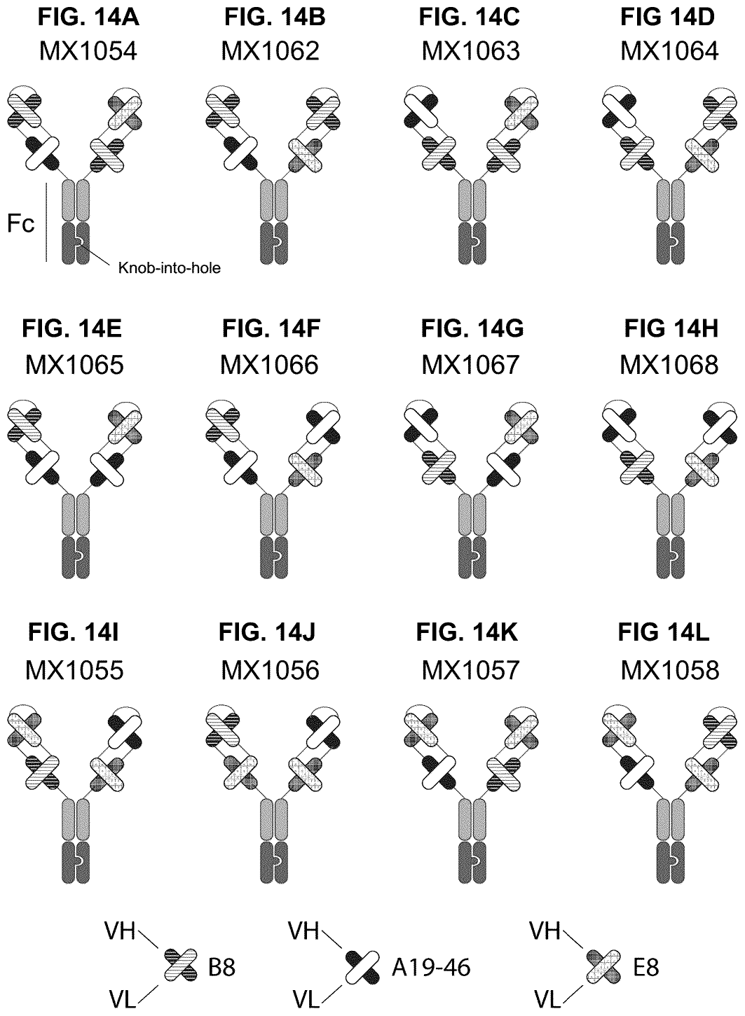

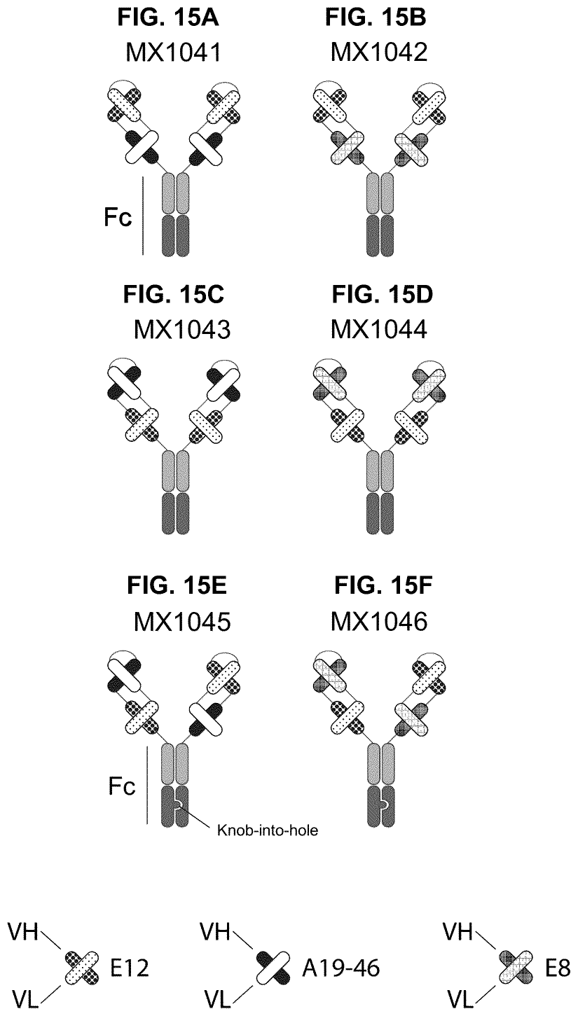

Additional multispecific antibodies were prepared using the methods of Example 1, having the structures shown in FIGS. 12A-12F, FIGS. 13A-13F, FIGS. 14A-14L, FIGS. 15A-15F, FIGS. 16A-16L, FIGS. 17A-17E and FIGS. 18A-18L. The pseudotyped neutralization potency (IC50 and IC80) of these antibodies was then determined using the methods described in Example 2.

Table 14 shows the IC50 and IC80 values (in nM) of the indicated molecules, or combination of molecules, for neutralization of Omicron sublineages BA.4/5, BQ.1, BQ.1.1, BA.2.75.2, XBB, XBB1.5 and BF.7.

TABLE 14

| | | Omicron Sublineages | | | | | | |
|---|---|---|---|---|---|---|---|---|
| | | BA.4/5 | | BQ 1 | | BQ 1.1 | | BA.2.75.2 |
| ID | Class | IC50 | IC80 | IC50 | IC80 | IC50 | IC80 | IC50 |
| MX1042 | I + III | 1.37 | 2.75 | 11.89 | 27.37 | 0.29 | 3.5 | 0.01 |
| MX1043 | I + II | 3.74 | 10.69 | 2.64 | 15.09 | 2.35 | 12.29 | 2.06 |
| MX1069 | I + II + III | 1.47 | 2.44 | 0.83 | 3.07 | 1.89 | 6.5 | 1.81 |
| MX1055 | I + II + III | 1.3 | 3.32 | 15.98 | 60.59 | 0.12 | 3.41 | 2.32 |
| MX1089 | I + II + III | 2.32 | 3.5 | 2.92 | 7.27 | 1.99 | 7.9 | 7.16 |
| E12 | I | 4.62 | 9.21 | 11.89 | 40.91 | 5.29 | 17.43 | 6.99 |
| B8 | I | 6.97 | 14.76 | 15.02 | 49.64 | 4.57 | 21.67 | 12.18 |
| A58VGLTG | I | 5.2 | 13.5 | 23.53 | 87.67 | 30.29 | 1316 | >10,000 |
| A46 | II | >10,000 | >10,000 | n.t. | n.t. | 386 | 1111 | >10,000 |
| E8 | III | 0.13 | 0.49 | 124 | 5191 | 496 | 1803 | 1.72 |
| LY1404 | III | 1.88 | 3.74 | n.t. | n.t. | >10,000 | >10,000 | 5.67 |
| A46 + E12 | I + II | 1.96 | 4.26 | 2.27 | 6.63 | 1.65 | 5.09 | 2.36 |
| E8 + E12 | I + III | 0.4 | 0.78 | 0.89 | 3.27 | 1.77 | 4.52 | 0.01 |
| A46 + E12 + B8 | I + II | n.t. | n.t. | n.t. | n.t. | n.t. | n.t. | n.t. |
| A46 + E12 + B8 + E8 | I + II + III | 1.36 | 5.82 | 1.69 | 15.69 | 8.10 | 19.17 | 1.47 |
| A46 + E8 + B8 | I + II + III | n.t. | n.t. | 16.03 | 52.16 | 11.14 | 17.02 | n.t. |
| A46 + E12 + E8 | I + II + III | n.t. | n.t. | n.t. | n.t. | n.t. | n.t. | n.t. |
| A46 + E12 + B8 + A58VGLTG | I + II + III | n.t. | n.t. | n.t. | n.t. | n.t. | n.t. | n.t. |
| A46 + E12 + E8 + A58VGLTG | I + II + III | n.t. | n.t. | n.t. | n.t. | n.t. | n.t. | n.t. |

| | Omicron Sublineages | | | | | | |
|---|---|---|---|---|---|---|---|
| | BA.2.75.2 | XBB | | XBB1.5 | | BF.7 | |
| ID | IC80 | IC50 | IC80 | IC50 | IC80 | IC50 | IC80 |
| MX1042 | 0.05 | 7.55 | 16.54 | 41.79 | 81.52 | 19.28 | 30.78 |
| MX1043 | 4.01 | 2.17 | 7.05 | 68.73 | 154.8 | 56.9 | 133.6 |
| MX1069 | 3.33 | 2.48 | 5.2 | 34.78 | 65.45 | 12.48 | 28.99 |
| MX1055 | 7.23 | 8.39 | 32.74 | 23.65 | 67.92 | 16.16 | 28.24 |
| MX1089 | 7.84 | 10.68 | 28.51 | 52.27 | 148.1 | 22.61 | 64.28 |
| E12 | 40.08 | 12.65 | 31.11 | 61.71 | 183.2 | 28.33 | 88.66 |
| B8 | 27.41 | 13.44 | 47.18 | 43.46 | 142.6 | 19.83 | 63.04 |
| A58VGLTG | >10,000 | >10,000 | >10,000 | >10,000 | >10,000 | 97.46 | 474.8 |
| A46 | >10,000 | n.t. | n.t. | >10,000 | >10,000 | 5296 | >10,000 |
| E8 | 5.9 | 61.5 | 705 | 284.4 | 318.8 | 15.56 | 30.92 |
| LY1404 | 20.72 | >10,000 | >10,000 | >10,000 | >10,000 | 3.28 | 26.94 |
| A46 + E12 | 4.68 | 2.76 | 9.01 | 57.75 | 126.9 | 38.27 | 67.65 |
| E8 + E12 | 0.05 | 2.96 | 7.82 | 17.89 | 86.65 | 11.38 | 47.97 |
| A46 + E12 + B8 | n.t. | n.t. | n.t. | 12 | 32.95 | 12.94 | 33.75 |
| A46 + E12 + B8 + E8 | 6.93 | 5.95 | 16.58 | n.t. | n.t. | n.t. | n.t. |
| A46 + E8 + B8 | n.t. | 11.29 | 17.7 | n.t. | n.t. | n.t. | n.t. |
| A46 + E12 + E8 | n.t. | n.t. | n.t. | 37.82 | 96.65 | 12.04 | 30.6 |
| A46 + E12 + B8 + A58VGLTG | n.t. | n.t. | n.t. | 15.28 | 30.59 | 17.35 | 20.02 |
| A46 + E12 + E8 + A58VGLTG | n.t. | n.t. | n.t. | 20.85 | 49.66 | 3.57 | 15.52 |

FIG. 19 shows the IC50 values (in nM) of the indicated molecules for neutralization of Omicron sublineages BA.1, BA.4/5, BA.2.12.1, BA.2.75, BA.4.6, BA.2.75.2, BQ.1.1, BJ.1 and XBB.

FIG. 20 shows the IC50 values (in nM) of the indicated molecules for neutralization of Omicron sublineages BA.4/5, BQ.1, BQ.1.1, BA.2.75.2 and XBB.

FIG. 21 shows the IC50 values (in nM) of the indicated molecules for neutralization of Omicron sublineages BA.4/5, BQ.1, BQ.1.1, BA.2.75.2 and XBB.

These results show that the tested molecules potently inhibited the Omicron variants, and to a much greater extent than bebtelovimab (LY1404).

### Example 7

### Pseudovirus Neutralization Assay

Additional multispecific antibodies were prepared using the methods of Example 1, having the structures shown in FIGS. 12A-12F, FIGS. 13A-13F, FIGS. 14A-14L, FIGS. 15A-15F, FIGS. 16A-16L, FIGS. 17A-17E and FIGS. 18A-18L. The pseudotyped neutralization potency (IC50 and IC80) of these antibodies was then determined using the methods described in Example 2.

Table 15 shows the IC50 and IC80 values (in ng/mL) of the indicated molecules, or combination of molecules, for neutralization of Omicron sublineages BA.4/5, BQ.1, BQ.1.1, BA.2.75.2, XBB, XBB1.5 and CH.1.1. The constructs tested included MX1054 (SEQ ID NOs:345, 347; 346, 348); MX1062 (SEQ ID NOs:349, 351; 350, 352); MX1063 (SEQ ID NOs:353, 355; 354, 356); MX1064 (SEQ ID NOs:357, 359; 358, 360); MX1065 (SEQ ID NOs:361, 363; 362, 364); MX1066 (SEQ ID NOs:365, 367; 366, 368); MX1067 (SEQ ID NOs:369, 371; 370, 372); MX1068 (SEQ ID NOs:373, 375; 374, 376); MX1055 (SEQ ID NOs:377, 379; 378, 380); MX1056 (SEQ ID NOs:381, 383; 382, 384); MX1057 (SEQ ID NOs:385, 387; 386, 388); and MX1058 (SEQ ID NOs:389, 391; 390, 392). These results show that the tested molecules potently inhibited the Omicron variants.

TABLE 15

| | | BA.4/5 | | BQ 1 | | BQ 1.1 | |
|---|---|---|---|---|---|---|---|
| ID | Class | IC50 | IC80 | IC50 | IC80 | IC50 | IC80 |
| MX1054 | I + II + III | 9.55 | 47.17 | 25.04 | 53.53 | 20.46 | 38.56 |
| MX1062 | I + II + III | 15.85 | 21.57 | 17.91 | 92.37 | 17.82 | 20.45 |
| MX1063 | I + II + III | 5.02 | 26.70 | 14.29 | 56.14 | 22.76 | 55.07 |
| MX1064 | I + II + III | 5.74 | 17.75 | 15.57 | 42.90 | 12.99 | 32.57 |
| MX1065 | I + II + III | n.t. | n.t. | 33.55 | 86.03 | 13.96 | 20.63 |
| MX1066 | I + II + III | n.t. | n.t. | 35.88 | 41.31 | 28.36 | 30.75 |
| MX1067 | I + II + III | 15.80 | 53.06 | 64.46 | 73.21 | 36.00 | 79.62 |
| MX1068 | I + II + III | 11.31 | 29.49 | 37.91 | 116.50 | 63.96 | 226.00 |
| MX1055 | I + II + III | 1.3 | 3.32 | 15.98 | 60.59 | 0.12 | 3.41 |
| MX1056 | I + II + III | 4.12 | 5.89 | 12.99 | 36.78 | 5.84 | 41.86 |
| MX1057 | I + II + III | 4.04 | 7.42 | 8.3 | 23.63 | 12.32 | 56.1 |
| MX1058 | I + II + III | 2.74 | 5.51 | 16 | 31.98 | 4.49 | 28.48 |
| A19-46 + E8 + B8 | I + II + III | n.t. | n.t. | 16.03 | 52.16 | 11.14 | 17.02 |
| B8 | I | 6.97 | 14.76 | 15.02 | 49.64 | 4.57 | 21.67 |
| E12 | I | 4.62 | 9.21 | 11.89 | 40.91 | 5.29 | 17.43 |
| E8 | III | 0.13 | 0.49 | 124 | 5191 | 496 | 1803 |
| A19-46 + E8 + B8 | II | >10,000 | >10,000 | n.t. | n.t. | 386 | 1111 |

| | BA.2.75.2 | | XBB | | XBB1.5 | | CH.1.1 | |
|---|---|---|---|---|---|---|---|---|
| ID | IC50 | IC80 | IC50 | IC80 | IC50 | IC80 | IC50 | IC80 |
| MX1054 | 13.12 | 35.44 | 17.66 | 29.87 | n.t. | n.t. | n.t. | n.t. |
| MX1062 | 16.18 | 34.50 | 18.28 | 39.26 | n.t. | n.t. | n.t. | n.t. |
| MX1063 | 8.41 | 21.04 | 6.62 | 18.10 | n.t. | n.t. | n.t. | n.t. |
| MX1064 | 14.39 | 35.12 | 6.12 | 24.72 | n.t. | n.t. | n.t. | n.t. |
| MX1065 | n.t. | n.t. | 12.5 | 16.74 | n.t. | n.t. | n.t. | n.t. |
| MX1066 | n.t. | n.t. | 28.74 | 29.75 | n.t. | n.t. | n.t. | n.t. |
| MX1067 | 4.34 | 10.72 | 26.34 | 74.07 | n.t. | n.t. | n.t. | n.t. |
| MX1068 | 7.60 | 25.00 | 15.16 | 46.85 | n.t. | n.t. | n.t. | n.t. |
| MX1055 | 2.32 | 7.23 | 8.39 | 32.74 | 23.65 | 67.92 | 125 | 862 |
| MX1056 | 2.53 | 9.92 | 7.13 | 23.63 | n.t. | n.t. | n.t. | n.t. |
| MX1057 | 1.27 | 3.79 | 6.39 | 35.53 | n.t. | n.t. | n.t. | n.t. |
| MX1058 | 4.03 | 10.02 | 14.16 | 31.79 | n.t. | n.t. | n.t. | n.t. |
| A19-46 + E8 + B8 | n.t. | n.t. | 11.29 | 17.7 | n.t. | n.t. | n.t. | n.t. |
| B8 | 12.18 | 27.41 | 13.44 | 47.18 | n.t. | n.t. | n.t. | n.t. |
| E12 | 6.99 | 40.08 | 12.65 | 31.11 | n.t. | n.t. | n.t. | n.t. |
| E8 | 1.72 | 5.9 | 61.5 | 705 | n.t. | n.t. | n.t. | n.t. |
| A19-46 + E8 + B8 | >10,000 | >10,000 | n.t. | n.t. | n.t. | n.t. | n.t. | n.t. |

Unit: ng/ml

Table 16 shows the IC50 and IC80 values (in ng/mL) of the indicated molecules, or combination of molecules, for neutralization of the original Wuhan strain (WA1), D614G spike protein mutant (D614G), Delta variant, and Omicron variants B.1.1.529, BA.4.6 and BF.7. These results show that the tested molecules potently inhibited the tested viruses.

TABLE 16

| | | | | | | | | BA.1.1.529 | | | | | |
|---|---|---|---|---|---|---|---|---|---|---|---|---|---|
| | | WA1 | | D614G | | Delta | | (Omicron) | | BA.4.6 | | BF.7 | |
| ID | Class | IC50 | IC80 | IC50 | IC80 | IC50 | IC80 | IC50 | IC80 | IC50 | IC80 | IC50 | IC80 |
| MX1054 | I + II + III | 15.84 | 66.29 | 13.83 | 30.95 | 6.08 | 57.02 | 2.15 | 7.20 | n.t. | n.t. | 11.74 | 28.17 |
| MX1062 | I + II + III | 54.66 | 73.09 | n.t. | n.t. | 111.50 | 160.00 | n.t. | n.t. | n.t. | n.t. | n.t. | n.t. |
| MX1063 | I + II + III | 6.55 | 38.84 | n.t. | n.t. | 53.87 | 288.20 | n.t. | n.t. | n.t. | n.t. | n.t. | n.t. |
| MX1064 | I + II + III | 64.75 | 73.72 | n.t. | n.t. | 17.36 | 51.15 | n.t. | n.t. | n.t. | n.t. | n.t. | n.t. |
| MX1065 | I + II + III | n.t. | n.t. | n.t. | n.t. | n.t. | n.t. | n.t. | n.t. | n.t. | n.t. | n.t. | n.t. |
| MX1066 | I + II + III | n.t. | n.t. | n.t. | n.t. | n.t. | n.t. | n.t. | n.t. | n.t. | n.t. | n.t. | n.t. |
| MX1067 | I + II + III | 59.58 | 68.07 | n.t. | n.t. | 2.23 | 254.30 | n.t. | n.t. | n.t. | n.t. | n.t. | n.t. |
| MX1068 | I + II + III | 8.31 | 24.68 | n.t. | n.t. | 30.42 | 282.10 | n.t. | n.t. | n.t. | n.t. | n.t. | n.t. |
| MX1055 | I + II + III | n.t. | n.t. | n.t. | n.t. | n.t. | n.t. | n.t. | n.t. | n.t. | n.t. | n.t. | n.t. |
| MX1056 | I + II + III | n.t. | n.t. | n.t. | n.t. | n.t. | n.t. | n.t. | n.t. | n.t. | n.t. | n.t. | n.t. |
| MX1057 | I + II + III | n.t. | n.t. | n.t. | n.t. | n.t. | n.t. | n.t. | n.t. | n.t. | n.t. | n.t. | n.t. |
| MX1058 | I + II + III | n.t. | n.t. | n.t. | n.t. | n.t. | n.t. | n.t. | n.t. | n.t. | n.t. | n.t. | n.t. |
| A19-46 + E8 + B8 | I + II + III | n.t. | n.t. | n.t. | n.t. | n.t. | n.t. | n.t. | n.t. | n.t. | n.t. | n.t. | n.t. |
| B8 | I | 71.92 | 84.07 | n.t. | n.t. | 13.27 | 198.60 | n.t. | n.t. | n.t. | n.t. | n.t. | n.t. |
| E12 | I | 77.82 | 118.10 | n.t. | n.t. | 17.25 | 32.11 | n.t. | n.t. | n.t. | n.t. | n.t. | n.t. |
| E8 | III | 8.77 | 20.73 | n.t. | n.t. | 6.81 | 33.32 | n.t. | n.t. | n.t. | n.t. | n.t. | n.t. |
| A19-46 + E8 + B8 | II | n.t. | n.t. | n.t. | n.t. | n.t. | n.t. | n.t. | n.t. | n.t. | n.t. | n.t. | n.t. |

Unit: ng/ml

Table 17 shows the IC50 and IC80 values (in ng/mL) of the indicated molecules for neutralization of Omicron variants BA.4/5, BQ.1, BQ.1.1, BA.2.75.2, XBB, XBB1.5 and CH.1.1. The constructs tested included MX1069 (SEQ ID NOs:257, 258; 393, 394); MX1070 (SEQ ID NOs:395, 397; 396, 398); MX1071 (SEQ ID NOs:399, 401; 400, 402); MX1072 (SEQ ID NOs:403, 405; 404, 406); MX1073 (SEQ ID NOs:407, 409; 408, 410); and MX1078 (SEQ ID NOs:427, 429; 428, 430). These results show that the tested molecules potently inhibited the Omicron variants.

TABLE 17

| | | BA.4/5 | | BQ 1 | | BQ 1.1 | |
|---|---|---|---|---|---|---|---|
| ID | Class | IC50 | IC80 | IC50 | IC80 | IC50 | IC80 |
| MX1069 | I + II + III | 1.47 | 2.44 | 0.83 | 3.07 | 1.89 | 6.5 |
| MX1070 | I + II + III | 2.49 | 3.58 | 4.64 | 9.16 | 4.06 | 11.01 |
| MX1071 | I + II + III | 5.77 | 15.41 | 4.38 | 33.24 | 16.65 | 52.73 |
| MX1072 | I + II + III | 4.46 | 17.82 | 6.32 | 16.66 | 15.42 | 25.87 |
| MX1073 | I + II + III | 2.78 | 4.28 | 1.9 | 10.84 | 5.77 | 24.06 |
| MX1078 | I + II + III | 3.72 | 17.92 | 33.44 | 210.20 | 48.40 | 91.84 |
| E12 | I | 4.62 | 9.21 | 11.89 | 40.91 | 5.29 | 17.43 |
| E8 | III | 0.13 | 0.49 | 124 | 5191 | 496 | 1803 |
| A19-46 | II | >10,000 | >10,000 | n.t. | n.t. | 386 | 1111 |

| | BA.2.75.2 | | XBB | | XBB1.5 | | CH.1.1 | |
|---|---|---|---|---|---|---|---|---|
| ID | IC50 | IC80 | IC50 | IC80 | IC50 | IC80 | IC50 | IC80 |
| MX1069 | 1.81 | 3.33 | 2.48 | 5.2 | 34.78 | 65.45 | 27.11 | 104 |
| MX1070 | 2.4 | 4.16 | 2.9 | 9.71 | n.t. | n.t. | n.t. | n.t. |
| MX1071 | 3.10 | 7.72 | 3.81 | 10.08 | n.t. | n.t. | n.t. | n.t. |
| MX1072 | 16.14 | 33.83 | 8.48 | 22.06 | n.t. | n.t. | n.t. | n.t. |
| MX1073 | 1.81 | 3.35 | 9.3 | 23.99 | n.t. | n.t. | n.t. | n.t. |
| MX1078 | 5.91 | 22.25 | 45.38 | 97.31 | n.t. | n.t. | n.t. | n.t. |
| E12 | 6.99 | 40.08 | 12.65 | 31.11 | n.t. | n.t. | n.t. | n.t. |
| E8 | 1.72 | 5.9 | 61.5 | 705 | n.t. | n.t. | n.t. | n.t. |
| A19-46 | >10,000 | >10,000 | n.t. | n.t. | n.t. | n.t. | n.t. | n.t. |

Unit: ng/ml

Table 18 shows the IC50 and IC80 values (in ng/mL) of the indicated molecules for neutralization of the original Wuhan strain (WA1), D614G spike protein mutant (D614G), Delta variant, and Omicron variants B.1.1.529, BA.4.6 and BF.7. These results show that the tested molecules potently inhibited the tested viruses.

TABLE 18

| | | | | | | | | BA.1.1.529 | | | | | |
|---|---|---|---|---|---|---|---|---|---|---|---|---|---|
| | | WA1 | | D614G | | Delta | | (Omicron) | | BA.4.6 | | BF.7 | |
| ID | Class | IC50 | IC80 | IC50 | IC80 | IC50 | IC80 | IC50 | IC80 | IC50 | IC80 | IC50 | IC80 |
| MX1069 | I + II + III | 33.21 | 38.17 | 5.47 | 23.87 | 40.06 | 47.59 | 1.72 | 5.45 | n.t. | n.t. | 12.48 | 28.99 |
| MX1070 | I + II + III | n.t. | n.t. | n.t. | n.t. | n.t. | n.t. | n.t. | n.t. | n.t. | n.t. | n.t. | n.t. |
| MX1071 | I + II + III | 33.07 | 49.94 | n.t. | n.t. | 9.19 | 47.15 | n.t. | n.t. | n.t. | n.t. | n.t. | n.t. |
| MX1072 | I + II + III | 40.50 | 68.15 | n.t. | n.t. | 24.83 | 107.30 | n.t. | n.t. | n.t. | n.t. | n.t. | n.t. |
| MX1073 | I + II + III | n.t. | n.t. | n.t. | n.t. | n.t. | n.t. | n.t. | n.t. | n.t. | n.t. | n.t. | n.t. |
| MX1078 | I + II + III | 15.10 | 46.44 | n.t. | n.t. | 11.88 | 25.93 | n.t. | n.t. | n.t. | n.t. | n.t. | n.t. |
| E12 | I | 77.82 | 118.10 | n.t. | n.t. | 17.25 | 32.11 | n.t. | n.t. | n.t. | n.t. | n.t. | n.t. |
| E8 | III | 8.77 | 20.73 | n.t. | n.t. | 6.81 | 33.32 | n.t. | n.t. | n.t. | n.t. | n.t. | n.t. |
| A19-46 | II | n.t. | n.t. | n.t. | n.t. | n.t. | n.t. | n.t. | n.t. | n.t. | n.t. | n.t. | n.t. |

Unit: ng/ml

Table 19 shows the IC50 and IC80 values (in ng/mL) of the indicated molecules or combination of molecules for neutralization of Omicron variants BA.4/5, BQ.1, BQ.1.1, BA.2.75.2, XBB, XBB1.5 and CH.1.1. The constructs tested included MX1086 (SEQ ID NOs:487, 489; 488, 490); MX1087 (SEQ ID NOs:495, 497; 496, 498); MX1089 (SEQ ID NOs:499, 501; 500, 502); and MX1206 (SEQ ID NOs:503, 505; 504, 506). These results show that the tested molecules potently inhibited the Omicron variants.

TABLE 19

| | | BA.4/5 | | BQ 1 | | BQ 1.1 | | BA.2.75.2 |
|---|---|---|---|---|---|---|---|---|
| ID | Class | IC50 | IC80 | IC50 | IC80 | IC50 | IC80 | IC50 |
| MX1086 | I + II + III | 1.95 | 2.99 | 2.69 | 8.21 | 3.57 | 10.26 | 1.43 |
| MX1087 | I + II + III | 1.65 | 2.68 | 2.38 | 5.97 | 2.56 | 6.72 | 2.59 |
| MX1089 | I + II + III | 2.32 | 3.5 | 2.92 | 7.27 | 1.99 | 7.9 | 7.16 |
| MX1206 | I + II + III | 13.21 | 25.97 | 21.42 | 46.49 | 12.20 | 32.05 | 4.82 |
| B8 | I | 6.97 | 14.76 | 15.02 | 49.64 | 4.57 | 21.67 | 12.18 |
| E12 | I | 4.62 | 9.21 | 11.89 | 40.91 | 5.29 | 17.43 | 6.99 |
| E8 | III | 0.13 | 0.49 | 124 | 5191 | 496 | 1803 | 1.72 |
| A46 | II | >10,000 | >10,000 | n.t. | n.t. | 386 | 1111 | >10,000 |
| A23-58L93VGLTG | I | 5.2 | 13.5 | 23.53 | 87.67 | 30.29 | 1316 | >10,000 |
| B8 + E8 + E12 + A19-46 | I + II + III | 1.36 | 5.82 | 1.69 | 15.69 | 8.10 | 19.17 | 1.47 |

| | BA.2.75.2 | XBB | | XBB1.5 | | CH.1.1 | |
|---|---|---|---|---|---|---|---|
| ID | IC80 | IC50 | IC80 | IC50 | IC80 | IC50 | IC80 |
| MX1086 | 3.3 | 2.67 | 5.89 | n.t. | n.t. | n.t. | n.t. |
| MX1087 | 3.57 | 3.09 | 5.54 | n.t. | n.t. | n.t. | n.t. |
| MX1089 | 7.84 | 10.68 | 28.51 | 52.27 | 148 | 175 | 613 |
| MX1206 | 10.53 | 58.16 | 90.66 | 64.96 | 77.30 | 217 | 624 |
| B8 | 27.41 | 13.44 | 47.18 | n.t. | n.t. | n.t. | n.t. |
| E12 | 40.08 | 12.65 | 31.11 | n.t. | n.t. | n.t. | n.t. |
| E8 | 5.9 | 61.5 | 705 | n.t. | n.t. | n.t. | n.t. |
| A46 | >10,000 | n.t. | n.t. | n.t. | n.t. | n.t. | n.t. |
| A23-58L93VGLTG | >10,000 | >10,000 | >10,000 | n.t. | n.t. | n.t. | n.t. |
| B8 + E8 + E12 + A19-46 | 6.93 | 5.95 | 16.58 | n.t. | n.t. | n.t. | n.t. |

Unit: ng/ml

Table 20 shows the IC50 and IC80 values (in ng/mL) of the indicated molecules for neutralization of the original Wuhan strain (WA1), D614G spike protein mutant (D614G), Delta variant, and Omicron variants B.1.1.529, BA.4.6 and BF.7. These results show that the tested molecules potently inhibited the tested viruses.

TABLE 20

| | | | | | | | | BA.1.1.529 | | | | | |
|---|---|---|---|---|---|---|---|---|---|---|---|---|---|
| | | WA1 | | D614G | | Delta | | (Omicron) | | BA.4.6 | | BF.7 | |
| ID | Class | IC50 | IC80 | IC50 | IC80 | IC50 | IC80 | IC50 | IC80 | IC50 | IC80 | IC50 | IC80 |
| MX1086 | I + II + III | 5.09 | 28.06 | 18.48 | 44.17 | 39.81 | 46.45 | 2.91 | 7.73 | n.t. | n.t. | 8.49 | 22.28 |
| MX1087 | I + II + III | 20.49 | 72.67 | 23.52 | 51.78 | 17.59 | 31.98 | 1.82 | 5.24 | n.t. | n.t. | 9.06 | 31.45 |
| MX1089 | I + II + III | 36.79 | 68.74 | 13.16 | 29.38 | 78.38 | 123.40 | 2.71 | 8.34 | n.t. | n.t. | 22.61 | 64.28 |
| MX1206 | I + II + III | n.t. | n.t. | n.t. | n.t. | n.t. | n.t. | n.t. | n.t. | n.t. | n.t. | 20.05 | 36.37 |
| B8 | I | 71.92 | 84.07 | n.t. | n.t. | 13.27 | 198.60 | n.t. | n.t. | n.t. | n.t. | n.t. | n.t. |
| E12 | I | 77.82 | 118.10 | n.t. | n.t. | 17.25 | 32.11 | n.t. | n.t. | n.t. | n.t. | n.t. | n.t. |
| E8 | III | 8.77 | 20.73 | n.t. | n.t. | 6.81 | 33.32 | n.t. | n.t. | n.t. | n.t. | n.t. | n.t. |
| A46 | II | n.t. | n.t. | n.t. | n.t. | n.t. | n.t. | n.t. | n.t. | n.t. | n.t. | n.t. | n.t. |
| A23-58L93VGLTG | I | n.t. | n.t. | n.t. | n.t. | n.t. | n.t. | n.t. | n.t. | n.t. | n.t. | n.t. | n.t. |
| B8 + E8 + E12 + A19-46 | I + II + III | n.t. | n.t. | n.t. | n.t. | n.t. | n.t. | n.t. | n.t. | n.t. | n.t. | n.t. | n.t. |

Unit: ng/ml

Table 21 shows the IC50 and IC80 values (in ng/mL) of the indicated molecules or bebtelovimab (LY1404) for neutralization of Omicron variants BA.4/5, BQ.1, BQ.1.1, BA.2.75.2, XBB, XBB1.5 and CH.1.1. The constructs tested included MX1018 (SEQ ID NOs:291, 291; 292, 292); MX1029 (SEQ ID NOs:295, 295; 296, 296); MX1020 (SEQ ID NOs:297, 297; 298, 298); MX1019 (SEQ ID NOs:329, 329; 330, 330); MX1025 (SEQ ID NOs:331, 331; 332, 332); and MX1026 (SEQ ID NOs:333, 333; 334, 334). These results show that the tested molecules potently inhibited the Omicron variants, and to a much greater extent than LY1404.

TABLE 21

| | | BA.4/5 | | BQ 1 | | BQ 1.1 | |
|---|---|---|---|---|---|---|---|
| ID | Class | IC50 | IC80 | IC50 | IC80 | IC50 | IC80 |
| MX1018 | I | 10.68 | 58.12 | 51.26 | 98.26 | 4.33 | 11.9 |
| MX1029 | I | 8.62 | 34.08 | 54.08 | 69.43 | 3.79 | 6.78 |
| MX1020 | III | 3.6 | 7.94 | 7.78 | 17.41 | 2.19 | 3.61 |
| MX1019 | I | n.t. | n.t. | 24.08 | 61.09 | 14.24 | 24.5 |
| MX1025 | I | n.t. | n.t. | 29.76 | 43.05 | 30.28 | 34.95 |
| MX1026 | III | n.t. | n.t. | 425.2 | 2769 | 963.2 | 3936 |
| B8 | I | 6.97 | 14.76 | 15.02 | 49.64 | 4.57 | 21.67 |
| E12 | I | 4.62 | 9.21 | 11.89 | 40.91 | 5.29 | 17.43 |
| E8 | III | 0.13 | 0.49 | 124 | 5191 | 496 | 1803 |
| LY1404 | III | | | n.t. | n.t. | >10,000 | >10,000 |

| | BA.2.75.2 | | XBB | | XBB1.5 | | CH.1.1 | |
|---|---|---|---|---|---|---|---|---|
| ID | IC50 | IC80 | IC50 | IC80 | IC50 | IC80 | IC50 | IC80 |
| MX1018 | 4.09 | 10.97 | 6.6 | 20.1 | n.t. | n.t. | n.t. | n.t. |
| MX1029 | 4.17 | 7.07 | 2 | 8.66 | n.t. | n.t. | n.t. | n.t. |
| MX1020 | 1.56 | 4.22 | 1.22 | 5.72 | n.t. | n.t. | n.t. | n.t. |
| MX1019 | n.t. | n.t. | 13.06 | 20.55 | n.t. | n.t. | n.t. | n.t. |
| MX1025 | n.t. | n.t. | 32.79 | 32.23 | n.t. | n.t. | n.t. | n.t. |
| MX1026 | n.t. | n.t. | 1247 | 4536 | n.t. | n.t. | n.t. | n.t. |
| B8 | 12.18 | 27.41 | 13.44 | 47.18 | n.t. | n.t. | n.t. | n.t. |
| E12 | 6.99 | 40.08 | 12.65 | 31.11 | n.t. | n.t. | n.t. | n.t. |
| E8 | 1.72 | 5.9 | 61.5 | 705 | n.t. | n.t. | n.t. | n.t. |
| LY1404 | 5.67 | 20.72 | >10,000 | >10,000 | n.t. | n.t. | n.t. | n.t. |

Unit: ng/ml

Table 22 shows the IC50 and IC80 values (in ng/mL) of the indicated molecules for neutralization of the original Wuhan strain (WA1), D614G spike protein mutant (D614G), Delta variant, and Omicron variants B.1.1.529, BA.4.6 and BF.7. These results show that the tested molecules potently inhibited the tested viruses.

TABLE 22

| | | | | | | | | BA.1.1.529 | | | | | |
|---|---|---|---|---|---|---|---|---|---|---|---|---|---|
| | | WA1 | | D614G | | Delta | | (Omicron) | | BA.4.6 | | BF.7 | |
| ID | Class | IC50 | IC80 | IC50 | IC80 | IC50 | IC80 | IC50 | IC80 | IC50 | IC80 | IC50 | IC80 |
| MX1018 | I | n.t. | n.t. | 10.68 | 58.12 | 51.26 | 98.26 | 4.33 | 11.9 | 4.09 | 10.97 | 6.6 | 20.1 |
| MX1029 | I | n.t. | n.t. | 8.62 | 34.08 | 54.08 | 69.43 | 3.79 | 6.78 | 4.17 | 7.07 | 2 | 8.66 |
| MX1020 | III | n.t. | n.t. | 3.6 | 7.94 | 7.78 | 17.41 | 2.19 | 3.61 | 1.56 | 4.22 | 1.22 | 5.72 |
| MX1019 | I | n.t. | n.t. | n.t. | n.t. | n.t. | n.t. | n.t. | n.t. | n.t. | n.t. | n.t. | n.t. |
| MX1025 | I | n.t. | n.t. | n.t. | n.t. | n.t. | n.t. | n.t. | n.t. | n.t. | n.t. | n.t. | n.t. |
| MX1026 | III | n.t. | n.t. | n.t. | n.t. | n.t. | n.t. | n.t. | n.t. | n.t. | n.t. | n.t. | n.t. |
| B8 | I | n.t. | n.t. | n.t. | n.t. | n.t. | n.t. | n.t. | n.t. | n.t. | n.t. | n.t. | n.t. |
| E12 | I | n.t. | n.t. | n.t. | n.t. | n.t. | n.t. | n.t. | n.t. | n.t. | n.t. | n.t. | n.t. |
| E8 | III | n.t. | n.t. | n.t. | n.t. | n.t. | n.t. | n.t. | n.t. | n.t. | n.t. | n.t. | n.t. |
| LY1404 | III | n.t. | n.t. | n.t. | n.t. | n.t. | n.t. | n.t. | n.t. | n.t. | n.t. | n.t. | n.t. |

Unit: ng/ml

Table 23 shows the IC50 and IC80 values (in ng/mL) of the indicated molecules, combination of molecules, or bebtelovimab (LY1404) for neutralization of Omicron variants BA.4/5, BQ.1, BQ.1.1, BA.2.75.2, XBB, XBB1.5 and CH.1. The constructs tested included MX1021 (SEQ ID NOs:299, 299; 300, 300); MX1022 (SEQ ID NOs:301, 301; 302, 302); MX1023 (SEQ ID NOs:303, 303; 304, 304); MX1024 (SEQ ID NOs:305, 305; 306, 306); MX1027 (SEQ ID NOs:307, 309; 308, 310); and MX1028 (SEQ ID NOs:311, 313; 312, 314). These results show that the tested molecules potently inhibited the Omicron variants, and to a much greater extent than LY1404.

TABLE 23

| | | BA.4/5 | | BQ 1 | | BQ 1.1 | |
|---|---|---|---|---|---|---|---|
| ID | Class | IC50 | IC80 | IC50 | IC80 | IC50 | IC80 |
| MX1021 | I + II | 13.79 | 26 | 11.34 | 51.89 | 2.77 | 28.49 |
| MX1022 | I + III | 1.98 | 3.33 | 6.58 | 19.2 | 1.13 | 6.72 |
| MX1023 | I + II | 29.66 | 30.72 | 29.72 | 85.18 | 28.38 | 95.98 |
| MX1024 | I + III | n.t. | n.t. | 17.56 | 37.09 | 12.07 | 16.93 |
| MX1027 | I + II | n.t. | n.t. | 21.6 | 64.86 | 13.51 | 23.24 |
| MX1028 | I + III | 1.81 | 3.73 | 10.48 | 25.46 | 4.2 | 14.38 |
| B8 | I | 6.97 | 14.76 | 15.02 | 49.64 | 4.57 | 21.67 |
| E8 | III | 0.13 | 0.49 | 124 | 5191 | 496 | 1803 |
| A19-46 | II | >10,000 | >10,000 | n.t. | n.t. | 386 | 1111 |
| A46 + B8 | II + III | n.t. | n.t. | 18.57 | 49.26 | 14.46 | 27.32 |
| E8 + B8 | I + III | n.t. | n.t. | 11.04 | 30.54 | 9.55 | 13.23 |
| LY1404 | III | | | n.t. | n.t. | >10,000 | >10,000 |

| | BA.2.75.2 | | XBB | | XBB1.5 | | CH.1.1 | |
|---|---|---|---|---|---|---|---|---|
| ID | IC50 | IC80 | IC50 | IC80 | IC50 | IC80 | IC50 | IC80 |
| MX1021 | 3.73 | 13.22 | 14.66 | 61.12 | n.t. | n.t. | n.t. | n.t. |
| MX1022 | 0.89 | 2.8 | 5.85 | 12.44 | n.t. | n.t. | n.t. | n.t. |
| MX1023 | 11.07 | 29.49 | 9.79 | 53.23 | n.t. | n.t. | n.t. | n.t. |
| MX1024 | n.t. | n.t. | 10.52 | 12.92 | n.t. | n.t. | n.t. | n.t. |
| MX1027 | n.t. | n.t. | 12.87 | 20.69 | n.t. | n.t. | n.t. | n.t. |
| MX1028 | 2.43 | 7.4 | 6.77 | 16.14 | n.t. | n.t. | n.t. | n.t. |
| B8 | 12.18 | 27.41 | 13.44 | 47.18 | n.t. | n.t. | n.t. | n.t. |
| E8 | 1.72 | 5.9 | 61.5 | 705 | n.t. | n.t. | n.t. | n.t. |
| A19-46 | >10,000 | >10,000 | n.t. | n.t. | n.t. | n.t. | n.t. | n.t. |
| A46 + B8 | n.t. | n.t. | 18.55 | 48.06 | n.t. | n.t. | n.t. | n.t. |
| E8 + B8 | n.t. | n.t. | 9.962 | 18.05 | n.t. | n.t. | n.t. | n.t. |
| LY1404 | 5.67 | 20.72 | >10,000 | >10,000 | n.t. | n.t. | n.t. | n.t. |

Unit: ng/ml

Table 24 shows the IC50 and IC80 values (in ng/mL) of the indicated molecules for neutralization of the original Wuhan strain (WA1), D614G spike protein mutant (D614G), Delta variant, and Omicron variants B.1.1.529, BA.4.6 and BF.7. These results show that the tested molecules potently inhibited the tested viruses.

TABLE 24

| | | | | | | | | BA.1.1.529 | | | | | |
|---|---|---|---|---|---|---|---|---|---|---|---|---|---|
| | | WA1 | | D614G | | Delta | | (Omicron) | | BA.4.6 | | BF.7 | |
| ID | Class | IC50 | IC80 | IC50 | IC80 | IC50 | IC80 | IC50 | IC80 | IC50 | IC80 | IC50 | IC80 |
| MX1021 | I + II | n.t. | n.t. | | | 64.83 | 218.6 | 2.91 | 13.08 | 12.95 | 41.4 | 11.33 | 31.67 |
| MX1022 | I + III | n.t. | n.t. | 5.15 | 13.33 | 31.53 | 25.37 | 4.22 | 4.74 | 4.9 | 8.82 | 2.38 | 8.29 |
| MX1023 | I + II | 15.31 | 82.37 | 22.87 | 40.6 | 88.89 | 205.4 | 3.74 | 8.67 | 23.44 | 77.55 | 18.67 | 54.51 |
| MX1024 | I + III | 1.76 | 15.65 | 1.76 | 8.16 | 32.93 | 37.13 | 1.63 | 5.44 | n.t. | n.t. | 3.12 | 12.50 |
| MX1027 | I + II | n.t. | n.t. | n.t. | n.t. | n.t. | n.t. | n.t. | n.t. | n.t. | n.t. | n.t. | n.t. |
| MX1028 | I + III | n.t. | n.t. | n.t. | n.t. | n.t. | n.t. | n.t. | n.t. | n.t. | n.t. | n.t. | n.t. |
| B8 | I | n.t. | n.t. | n.t. | n.t. | n.t. | n.t. | n.t. | n.t. | n.t. | n.t. | n.t. | n.t. |
| E8 | III | n.t. | n.t. | n.t. | n.t. | n.t. | n.t. | n.t. | n.t. | n.t. | n.t. | n.t. | n.t. |
| A19-46 | II | n.t. | n.t. | n.t. | n.t. | n.t. | n.t. | n.t. | n.t. | n.t. | n.t. | n.t. | n.t. |
| A46 + B8 | II + III | n.t. | n.t. | n.t. | n.t. | n.t. | n.t. | n.t. | n.t. | n.t. | n.t. | n.t. | n.t. |
| E8 + B8 | I + III | n.t. | n.t. | n.t. | n.t. | n.t. | n.t. | n.t. | n.t. | n.t. | n.t. | n.t. | n.t. |
| LY1404 | III | n.t. | n.t. | n.t. | n.t. | n.t. | n.t. | n.t. | n.t. | n.t. | n.t. | n.t. | n.t. |

Unit: ng/ml

Table 25 shows the IC50 and IC80 values (in ng/mL) of the indicated molecules or combination of molecules for neutralization of Omicron variants BA.4/5, BQ.1, BQ.1.1, BA.2.75.2, XBB, XBB1.5 and CH.1.1. The constructs tested included MX1041 (SEQ ID NOs:315, 315; 316, 316); MX1042 (SEQ ID NOs:256, 256; 507, 507); MX1043 (SEQ ID NOs:317, 317; 318, 318); MX1045 (SEQ ID NOs:321, 323; 322, 324); and MX1046 (SEQ ID NOs:325, 327; 326, 328). These results show that the tested molecules potently inhibited the Omicron variants.

TABLE 25

| | | BA.4/5 | | BQ 1 | | BQ 1.1 | |
|---|---|---|---|---|---|---|---|
| ID | Class | IC50 | IC80 | IC50 | IC80 | IC50 | IC80 |
| MX1041 | I + II | 12.04 | 26.23 | 42.86 | 188 | 49.25 | 110 |
| MX1042 | I + III | 1.37 | 2.75 | 11.89 | 27.37 | 0.29 | 3.5 |
| MX1043 | I + II | 3.74 | 10.69 | 2.64 | 15.09 | 2.35 | 12.29 |
| MX1045 | I + II | 2.68 | 6.06 | 6.02 | 22.31 | 1.5 | 8.57 |
| MX1046 | I + III | 0.9 | 1.56 | 1.66 | 4.08 | 1.23 | 3.97 |
| E12 | I | 4.62 | 9.21 | 11.89 | 40.91 | 5.29 | 17.43 |
| E8 | III | 0.13 | 0.49 | 124 | 5191 | 496 | 1803 |
| A46 | II | >10,000 | >10,000 | n.t. | n.t. | 386 | 1111 |
| A19-46 + E12 | I + II | 1.96 | 4.26 | 2.27 | 6.63 | 1.65 | 5.09 |
| E8 + E12 | I + III | 0.4 | 0.78 | 0.89 | 3.27 | 1.77 | 4.52 |

| | BA.2.75.2 | | XBB | | XBB1.5 | | CH.1.1 | |
|---|---|---|---|---|---|---|---|---|
| ID | IC50 | IC80 | IC50 | IC80 | IC50 | IC80 | IC50 | IC80 |
| MX1041 | 12.88 | 36.25 | 69.09 | 398 | n.t. | n.t. | n.t. | n.t. |
| MX1042 | 0.01 | 0.05 | 7.55 | 16.54 | 41.79 | 81.52 | 46.17 | 158 |
| MX1043 | 2.06 | 4.01 | 2.17 | 7.05 | 68.73 | 155 | 45.05 | 232 |
| MX1045 | 2.91 | 5.43 | 6.09 | 18.04 | n.t. | n.t. | n.t. | n.t. |
| MX1046 | 0.36 | 1.29 | 1.26 | 2.88 | n.t. | n.t. | n.t. | n.t. |
| E12 | 6.99 | 40.08 | 12.65 | 31.11 | n.t. | n.t. | n.t. | n.t. |
| E8 | 1.72 | 5.9 | 61.5 | 705 | n.t. | n.t. | n.t. | n.t. |
| A46 | >10,000 | >10,000 | n.t. | n.t. | n.t. | n.t. | n.t. | n.t. |
| A19-46 + E12 | 2.36 | 4.68 | 2.76 | 9.01 | n.t. | n.t. | n.t. | n.t. |
| E8 + E12 | 0.01 | 0.05 | 2.96 | 7.82 | n.t. | n.t. | n.t. | n.t. |

Unit: ng/ml

Table 26 shows the IC50 and IC80 values (in ng/mL) of the indicated molecules for neutralization of the D614G spike protein mutant (D614G), Delta variant, and Omicron variants B.1.1.529, BA.4.6 and BF.7. These results show that the tested molecules potently inhibited the tested viruses.

TABLE 26

| | | | | | | | | BA.1.1.529 | | | | | |
|---|---|---|---|---|---|---|---|---|---|---|---|---|---|
| | | WA1 | | D614G | | Delta | | (Omicron) | | BA.4.6 | | BF.7 | |
| ID | Class | IC50 | IC80 | IC50 | IC80 | IC50 | IC80 | IC50 | IC80 | IC50 | IC80 | IC50 | IC80 |
| MX1041 | I + II | n.t. | n.t. | 7.69 | 45.67 | 29.05 | 48.51 | 4.14 | 8.81 | 25.72 | 67.06 | 8.35 | 30.38 |
| MX1042 | I + III | n.t. | n.t. | 6.03 | 12.17 | 41.69 | 61.43 | 2.56 | 4.34 | 2.1 | 4.51 | 2.73 | 6.74 |
| MX1043 | I + II | n.t. | n.t. | n.t. | n.t. | n.t. | n.t. | n.t. | n.t. | n.t. | n.t. | n.t. | n.t. |
| MX1045 | I + II | n.t. | n.t. | n.t. | n.t. | n.t. | n.t. | n.t. | n.t. | n.t. | n.t. | n.t. | n.t. |
| MX1046 | I + III | n.t. | n.t. | n.t. | n.t. | n.t. | n.t. | n.t. | n.t. | n.t. | n.t. | n.t. | n.t. |
| E12 | I | n.t. | n.t. | n.t. | n.t. | n.t. | n.t. | n.t. | n.t. | n.t. | n.t. | n.t. | n.t. |
| E8 | III | n.t. | n.t. | n.t. | n.t. | n.t. | n.t. | n.t. | n.t. | n.t. | n.t. | n.t. | n.t. |
| A46 | II | n.t. | n.t. | n.t. | n.t. | n.t. | n.t. | n.t. | n.t. | n.t. | n.t. | n.t. | n.t. |
| A19-46 + E12 | I + II | n.t. | n.t. | n.t. | n.t. | n.t. | n.t. | n.t. | n.t. | n.t. | n.t. | n.t. | n.t. |
| E8 + E12 | I + III | n.t. | n.t. | n.t. | n.t. | n.t. | n.t. | n.t. | n.t. | n.t. | n.t. | n.t. | n.t. |

Unit: ng/ml

Table 27 shows the IC50 and IC80 values (in ng/mL) of the indicated molecules for neutralization of Omicron variants BA.4/5, BQ.1, BQ.1.1, BA.2.75.2, XBB, XBB1.5 and CH.1.1. The constructs tested included MX1047 (SEQ ID NOs:335, 337; 336, 338); and MX1049 (SEQ ID NOs:339, 339; 340, 340). These results show that the tested molecules potently inhibited the Omicron variants.

TABLE 27

| | | BA.4/5 | | BQ 1 | | BQ 1.1 | | BA.2.75.2 |
|---|---|---|---|---|---|---|---|---|
| ID | Class | IC50 | IC80 | IC50 | IC80 | IC50 | IC80 | IC50 |
| MX1047 | I + III | 3.46 | 8.46 | 19.72 | 190 | 96.68 | 700 | 4.36 |
| MX1049 | I + III | 3.47 | 10.64 | 22.65 | 103 | 64.5 | 2177 | 6.36 |
| E8 | III | 0.13 | 0.49 | 124 | 5191 | 496 | 1803 | 1.72 |
| A23-58L93VGLTG | I | 5.2 | 13.5 | 23.53 | 87.67 | 30.29 | 1316 | >10,000 |

| | BA.2.75.2 | XBB | | XBB1.5 | | CH.1.1 | |
|---|---|---|---|---|---|---|---|
| ID | IC80 | IC50 | IC80 | IC50 | IC80 | IC50 | IC80 |
| MX1047 | 25.83 | 208 | 3291 | n.t. | n.t. | n.t. | n.t. |
| MX1049 | 17.13 | 156 | 4531 | n.t. | n.t. | n.t. | n.t. |
| E8 | 5.9 | 61.5 | 705 | n.t. | n.t. | n.t. | n.t. |
| A23-58L93VGLTG | >10,000 | >10,000 | >10,000 | n.t. | n.t. | n.t. | n.t. |

Unit: ng/ml

Table 28 shows the IC50 and IC80 values (in ng/mL) of the indicated molecules for neutralization of the D614G spike protein mutant (D614G), Delta variant, and Omicron variants B.1.1.529, BA.4.6 and BF.7. These results show that the tested molecules potently inhibited the tested viruses.

TABLE 28

| | | | | | | | | BA.1.1.529 | | | | | |
|---|---|---|---|---|---|---|---|---|---|---|---|---|---|
| | | WA1 | | D614G | | Delta | | (Omicron) | | BA.4.6 | | BF.7 | |
| ID | Class | IC50 | IC80 | IC50 | IC80 | IC50 | IC80 | IC50 | IC80 | IC50 | IC80 | IC50 | IC80 |
| MX1047 | I + III | n.t. | n.t. | 3.48 | 5.52 | 27.73 | 46.94 | 4.04 | 4.6 | 7.11 | 17.17 | 4.37 | 19.32 |
| MX1049 | I + III | n.t. | n.t. | 5.61 | 12.79 | 18.79 | 22.03 | 3.27 | 7.16 | 5.19 | 20.45 | 4.68 | 19.88 |
| E8 | III | n.t. | n.t. | n.t. | n.t. | n.t. | n.t. | n.t. | n.t. | n.t. | n.t. | n.t. | n.t. |
| A23-58L93VGLTG | I | n.t. | n.t. | n.t. | n.t. | n.t. | n.t. | n.t. | n.t. | n.t. | n.t. | n.t. | n.t. |

Unit: ng/ml

### Example 8

### Pseudovirus Neutralization Assay and Other Properties

The pseudotyped neutralization potency (IC50 and IC80) of additional antibodies was determined using the methods described in Example 2.

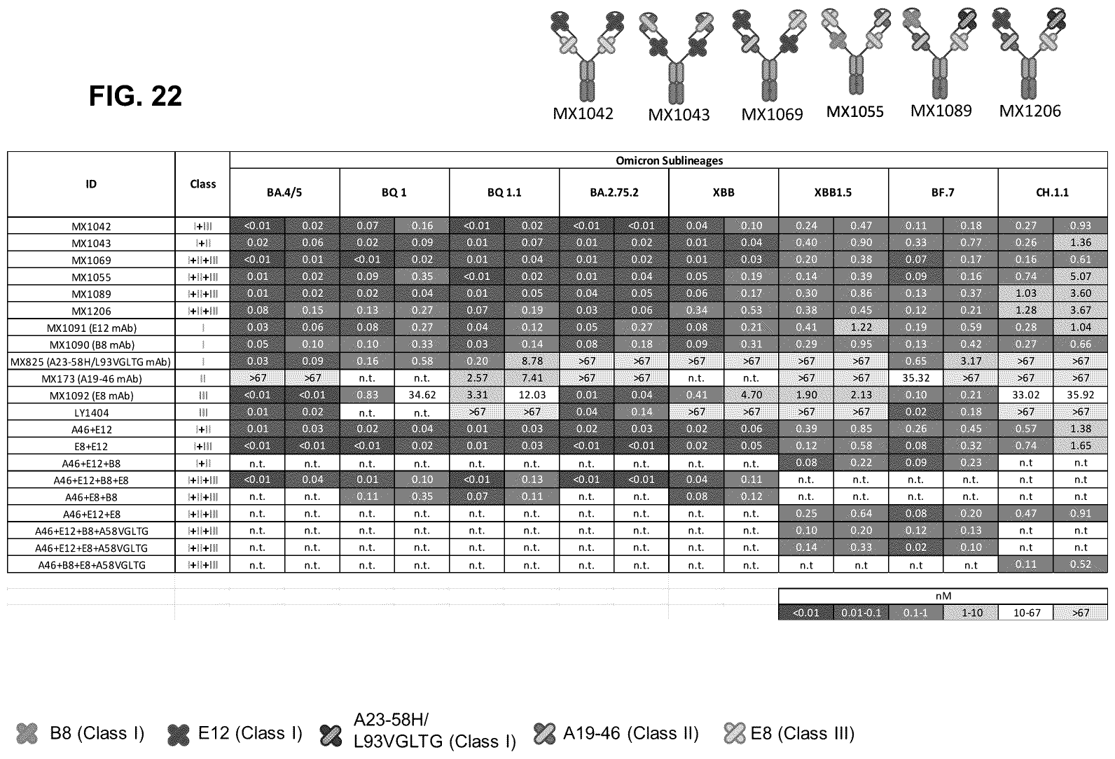

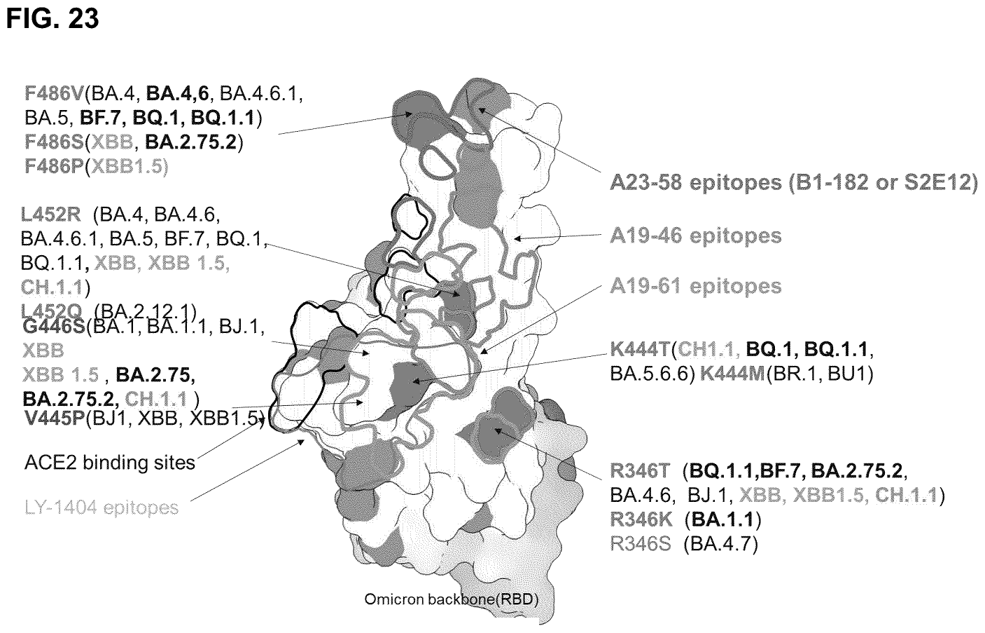

FIG. 22 shows the IC50 and IC80 values (in nM) of the indicated molecules, or combination of molecules, for neutralization of Omicron sublineages BA.4/5, BQ.1, BQ.1.1, BA.2.75.2, XBB, XBB1.5, BF.7 and CH.1.1. The antibody constructs tested included MX1042 (SEQ ID NOs:256, 256; 507, 507); MX1043 (SEQ ID NOs:317, 317; 318, 318); MX1069 (SEQ ID NOs:257, 258; 393, 394); MX1055 (SEQ ID NOs:377, 379; 378, 380); MX1089 (SEQ ID NOs:499, 501; 500, 502); and MX1206 (SEQ ID NOs:503, 505; 504, 506). These results show that the tested molecules potently inhibited the Omicron variants.

In addition, blood levels and pI were measured for MX1042, MX1043, MX1069, MX1089, MX1206 and MX1091. Table 29 shows pI values measured.

TABLE 29

| | MX1042 | MX1043 | MX1069 | MX1089 | MX1206 | MX1091 |
|---|---|---|---|---|---|---|
| pI | 6.92 | 6.99 | 6.73 | 8.36 | 7.8 | 7.92 |

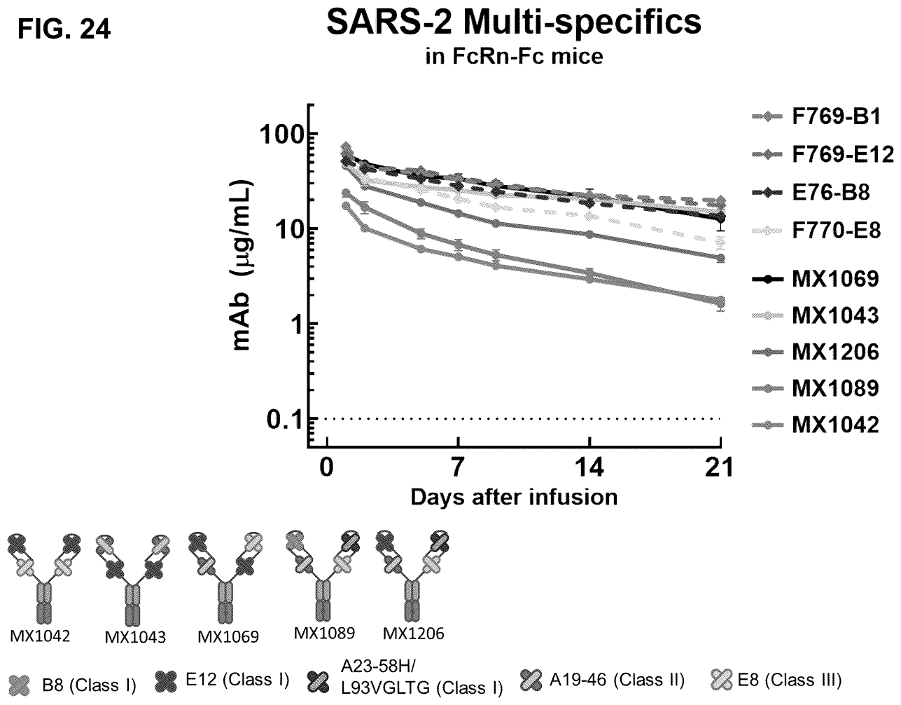

FIG. 24 shows the blood levels of these antibodies in Syrian hamsters up to 21 days after infusion.

### Example 9

### Tetravalent, Trispecific and Tetraspecific Antibodies

### Protect Syrian Hamsters from SARS-CoV-2 Variants

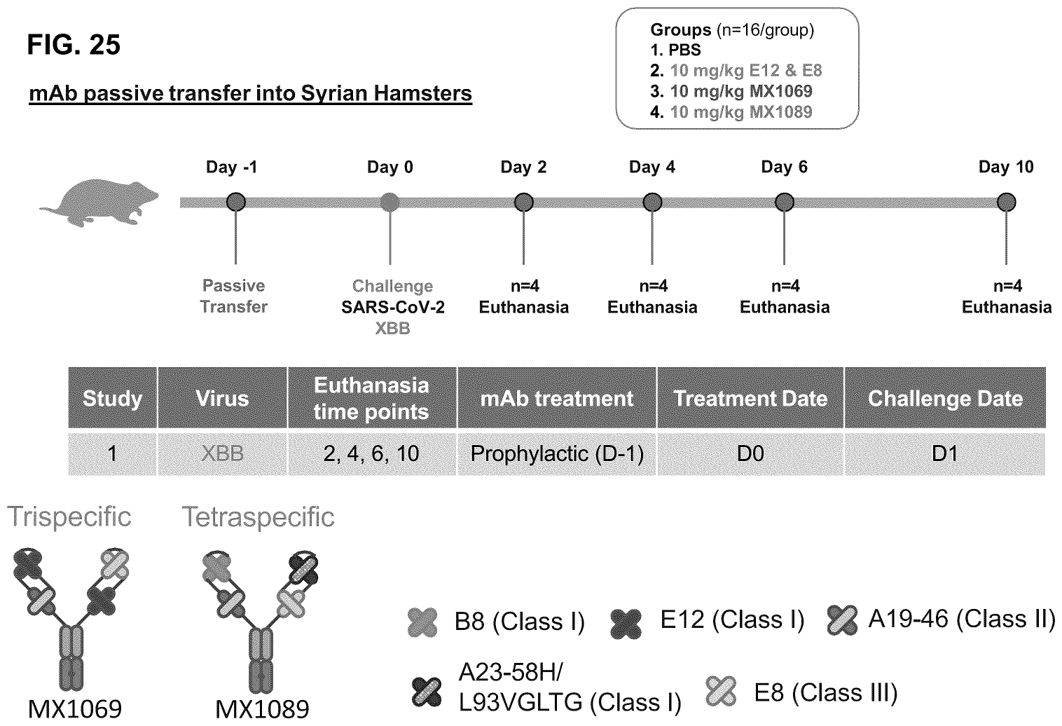

FIG. 25 shows a diagram of a passive transfer experiment that was performed in Syrian hamsters, the methods of which are explained further in Example 4. Animals were treated with 10 mg/kg MX1069 or 10 mg/kg MX1089, or with a combination of 10 mg/kg E12 and E8 or PBS as controls. Body weight was measured daily after challenge with Omicro variant XBB. In addition, viral load of the lungs and viral load of the nares were measured in TCID50/g as explained in Example 4.

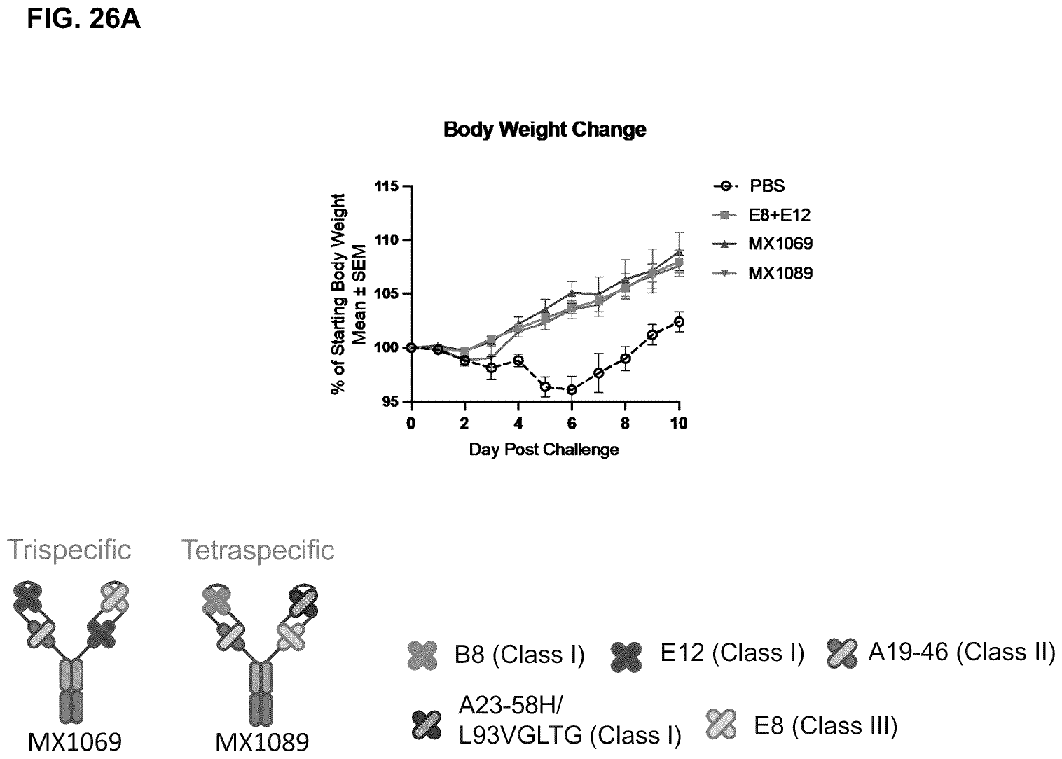

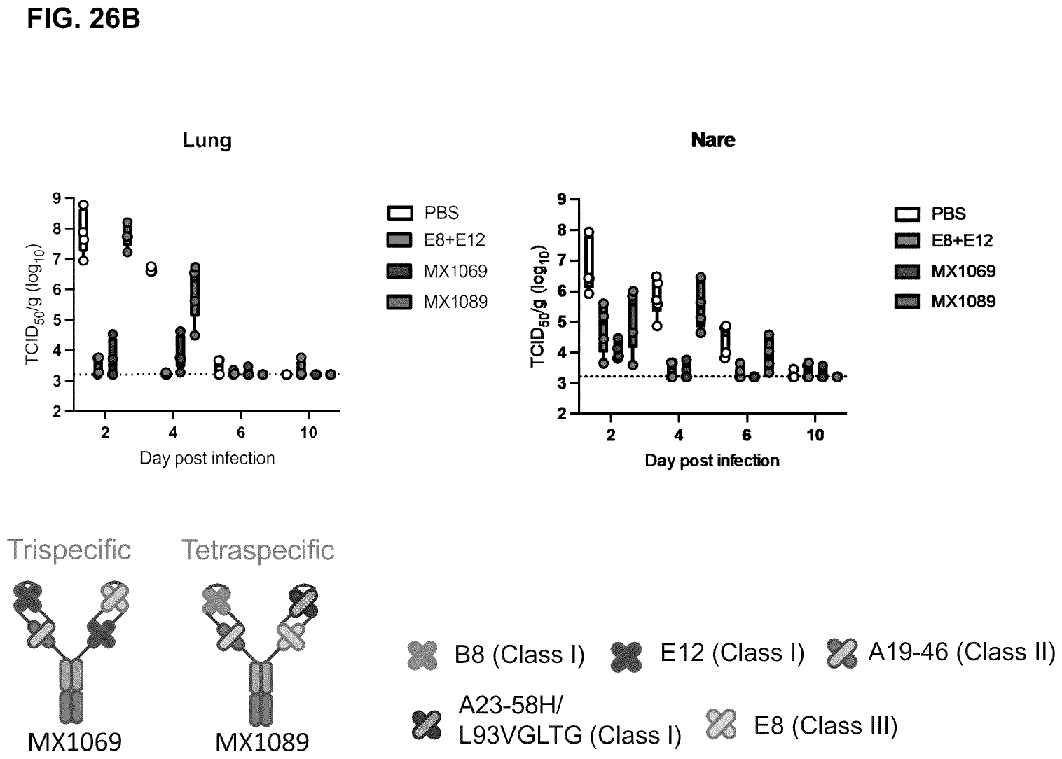

FIG. 26A shows the percent body weight change of XBB-infected animals treated with MX1069 and MX1089. These results show that both MX1069 and MX1089 protected animals from weight loss caused by XBB infection.

FIG. 26B shows the lung viral load (left panel) and nares viral load (right panel) of XXB-infected animals treated with MX1069 and MX1089. These results show that MX1069 reduced lung viral titers.

### Example 10

### Tetravalent, Bispecific Antibodies Protect Syrian Hamsters from SARS-CoV-2 Variants

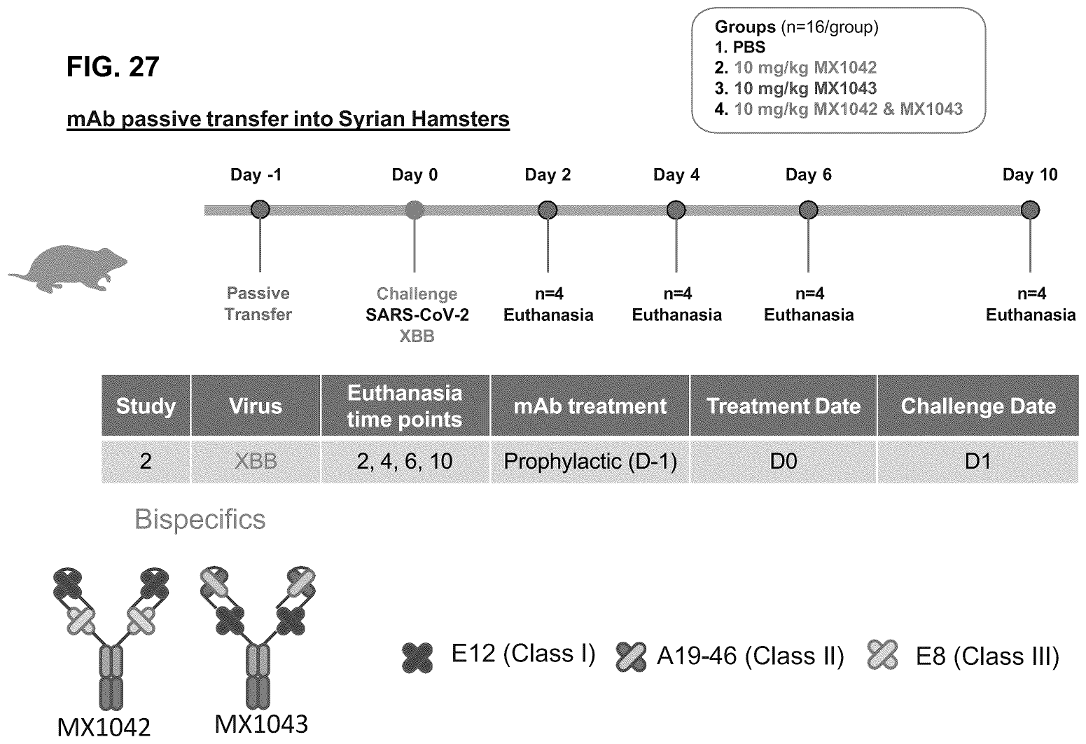

FIG. 27 shows a diagram of a passive transfer experiment that was performed in Syrian hamsters, the methods of which are explained further in Example 4. Animals were treated with 10 mg/kg MX1042, 10 mg/kg MX1043, a combination of 10 mg/kg MX1042 and MX1043, or PBS as a control. Body weight was measured daily after challenge with Omicro variant XBB. In addition, viral load of the lungs and viral load of the nares were measured in TCID50/g as explained in Example 4.

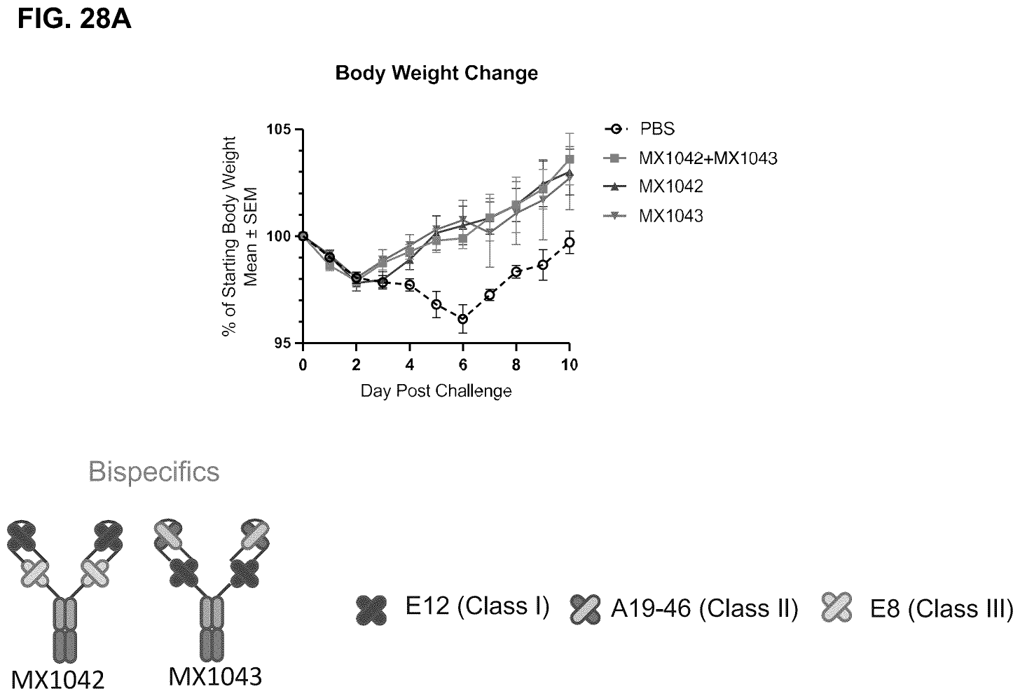

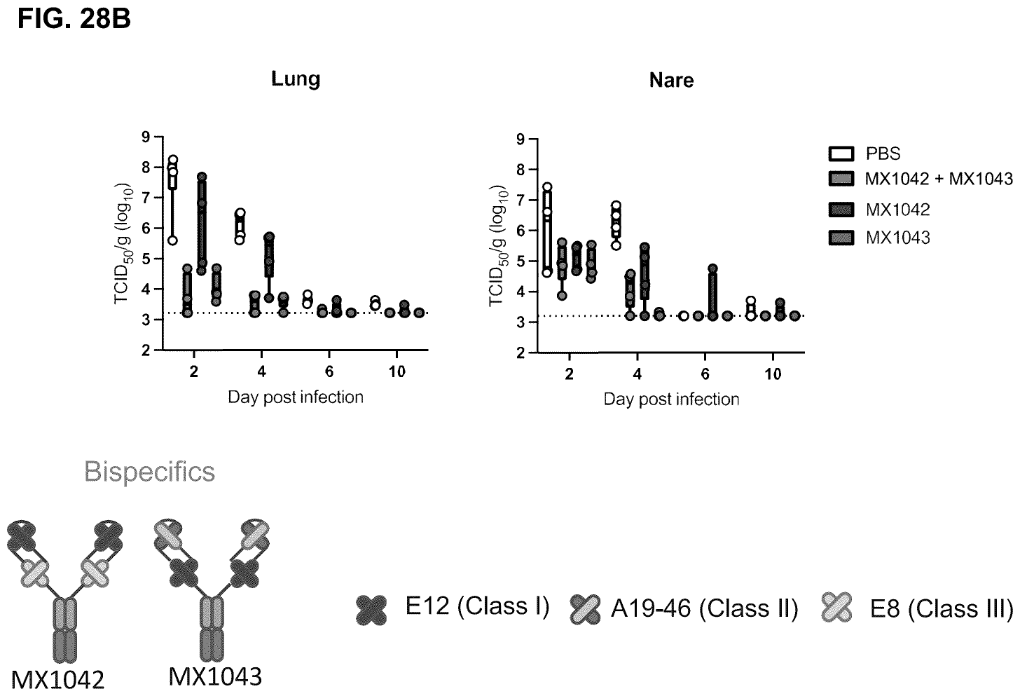

FIG. 28A shows the percent body weight change of XBB-infected animals treated with MX1042, MX1043 and a combination of MX1042 and MX1043. These results show that both MX1042 and MX1043 protected animals from weight loss caused by XBB infection.

FIG. 28B shows the lung viral load (left panel) and nares viral load (right panel) of XXB-infected animals treated with MX1042, MX1043 and a combination of MX1042 and MX1043. These results show that MX1043 and the combination of MX1043 and MX1042 reduced lung viral titers.

### Example 11

### Pseudovirus Neutralization Assay

A tetravalent, trispecific antibody, 46.1-182.1v/61.1-182.1v, was designed that contained single Fv46 and Fv61 domains and two stabilized 182.1 Fv (Fv182.1v) domains, using the methods of Example 1. The pseudotyped neutralization potency (IC50 and IC80) of these antibodies was then determined using the methods described in Example 2.

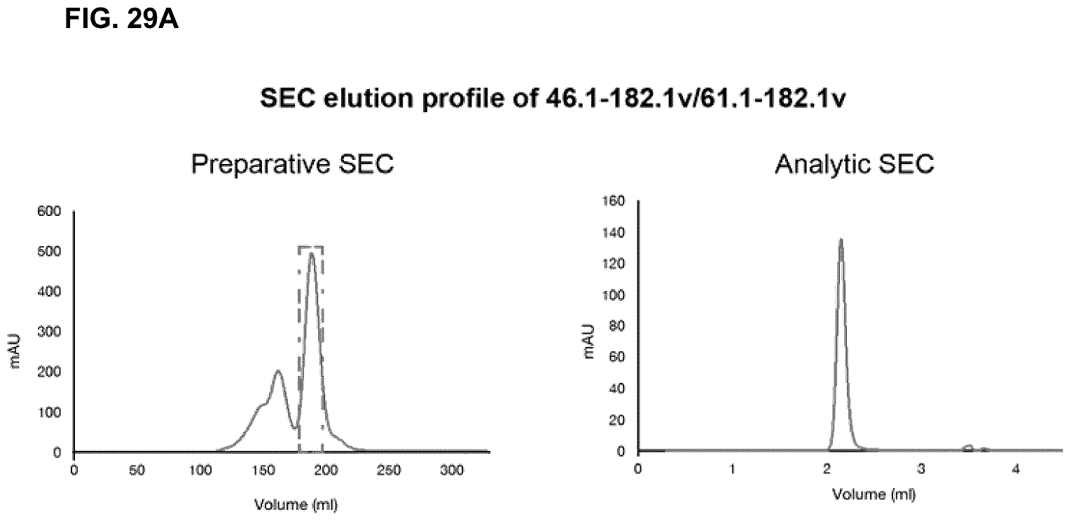

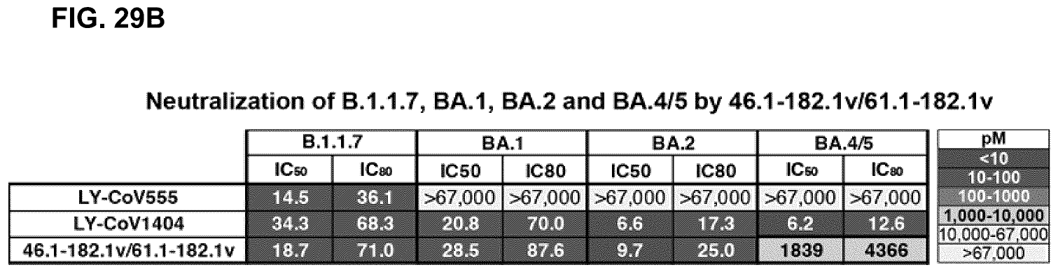

FIG. 29A shows the SEC elution profile of 46.1-182.1v/61.1-182.1v for preparative and analytic SEC. Specifically, traces from preparative SEC (pSEC, HiLoad 26/600 Superdex 200 pg, left) and analytic SEC (aSEC, Superdex 200 Increase 5/150GL, right) are shown after the combination of fractions collected from the boxed region of the preparative SEC gel. The box indicates fractions pooled from pSEC that were subsequently analyzed on the aSEC column. The same fractions were used for the hamster experiment.

FIG. 29B shows the IC50 and IC80 values (in pM) of the indicated molecules, for neutralization of Omicron sublineages B.1.1.7, BA.1, BA.2 and BA.4/5. These results show that 46.1-182.1v/61.1-182.1v potently inhibited the Omicron variants, and to a greater extent than Ly-CoV555 or LY-CoV1404.

### Example 12

### Lung Histopathology of BA.5-Infected Hamsters Treated with 46.1-182.1v/61.1-182.1v

To assess the clinical impact of 46.1-182.1v/61.1-182.1v on lung infection and pathology, a histopathologic examination was performed on lung tissue from BA.5-infected Syrian hamsters treated with PBS or antibody.

On days 2, 4, 6 and 10, four animals were euthanized and a histopathological examination was performed on the lungs. At necropsy, the left lung was collected and placed in 10% neutral buffered formalin for histopathologic analysis. Tissue sections were trimmed and processed to hematoxylin and eosin (H&E) stained slides and examined by a board-certified pathologist at Experimental Pathology Laboratories, Inc. (EPL®) in Sterling, Virginia.

Fixed lung was processed for immunohistochemical (IHC) detection of SARS-CoV-2 nucleocapsid protein, using a SARS-CoV-2 (COVID-19) nucleocapsid antibody on formalin-fixed paraffin-embedded tissue. Anti-SARS-CoV-2, 0.02 μg/mL, primary antibody (ProSci 9099; SARS-CoV-2 (COVID-19) Nucleocapsid Antibody) was applied to tissues overnight at 4° C. (±2° C.) followed by Polymer Anti-Rabbit Immunoglobulin G (IgG) Reagent (made in goat, ready-to-use [RTU]) for 30 minutes, 3,3′-Diaminobenzidine tetrahydrochloride (DAB) solution (3 minutes), and counterstained with hematoxylin. Background staining was equally observed in both control and treated animals and did not impede interpretation.

Representative images from histopathologic tissue sections stained with hematoxylin and eosin (H&E) (top rows) or immunostained with anti-SARS-CoV-2 nucleocapsid antibody (bottom rows) are shown herein.

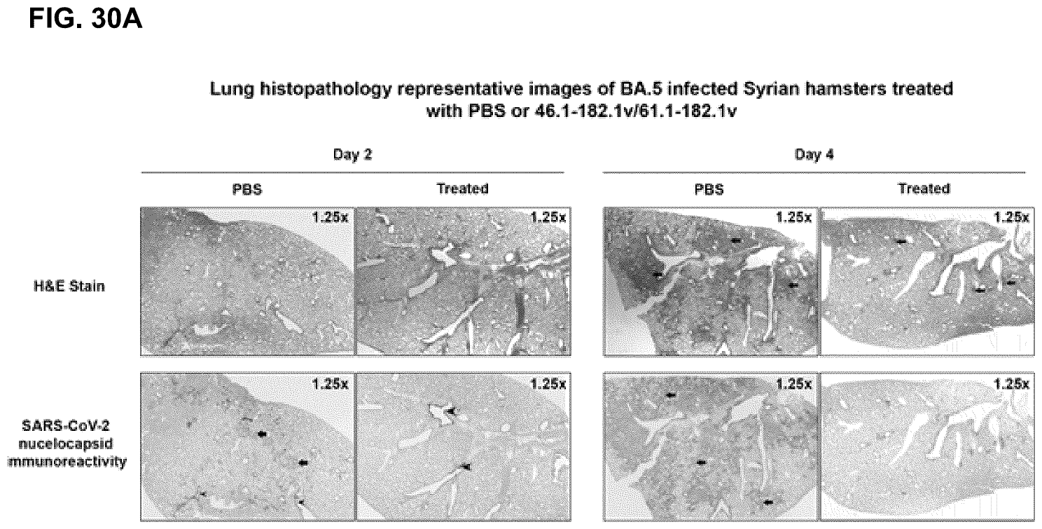

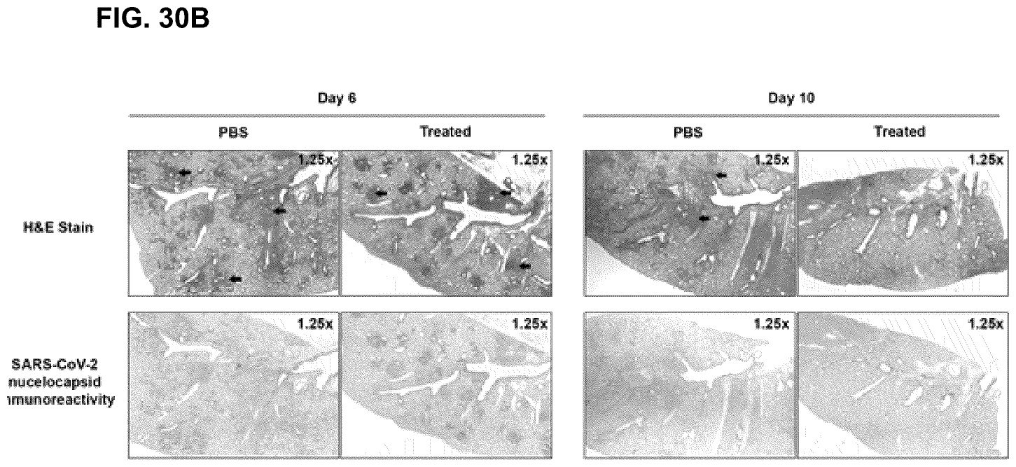

Results from Day 2 and Day 4 are shown in FIG. 30A.

Day 2. In PBS control and antibody-treated animals, SARS-CoV-2 (BA.5)-related microscopic findings were minimal to mild. Mild to moderate SARS-CoV-2 nucleocapsid immunoreactivity (intense dark brown staining) was observed in PBS controls, while minimal to mild immunoreactivity was observed in antibody-treated animals. Immunoreactivity was localized in Type II pneumocytes lining the alveolar septa (arrows) and bronchiolar epithelium (arrowheads).

Day 4. SARS-CoV-2 (BA.5)-related microscopic findings were observed in the lung of PBS control and antibody-treated animals. In H&E sections, affected areas are dark and consolidated (arrows). Severity and/or incidence of SARS-CoV-2 (BA.5)-related microscopic findings were decreased in antibody-treated animals compared to PBS controls. Mild to moderate SARS-CoV-2 nucleocapsid immunoreactivity (brown staining) was mainly observed in Type II pneumocytes lining alveolar septa in control animals. Immunoreactivity was observed in antibody-treated animals; however, the minimal to mild nature was not visible at low magnification. Faint brown discoloration in antibody-treated animals was considered background staining of inflammatory cells and not indicative of nucleocapsid reactivity.

Results from Day 6 and Day 10 are shown in FIG. 30B.

Day 6. There were SARS-CoV-2 (BA.5)-related microscopic findings observed in the lung of PBS control and antibody-treated animals. In H&E sections, affected areas were dark and consolidated (arrows). Severity and/or incidence of SARS-CoV-2 (BA.5)-related microscopic findings were decreased in antibody-treated animals compared to PBS controls. Minimal to mild SARS-CoV-2 nucleocapsid immunoreactivity was observed in Type II pneumocytes lining alveolar septa in PBS control animals; however, the minimal to mild nature was not visible at low magnification.

Day 10. There were no SARS-CoV-2 (BA.5)-related microscopic findings in the lung of animals treated with antibody. SARS-CoV-2 (BA.5)-related microscopic findings were observed in PBS control animals; affected areas were dark and consolidated (arrows). SARS-CoV-2 nucleocapsid immunoreactivity was not observed in any group.

Tables 30-33 show the lung histopathology scoring of BA.5-infected Syrian hamsters treated with PBS or 46.1-182.1v/61.1-182.1v. The severity of the microscopic finding and the number of animals noted to have the finding at that severity level are noted. Grading scales are as follows:

Minimal. This corresponds to a histopathologic change ranging from inconspicuous to barely noticeable but so minor, small, or infrequent as to warrant no more than the least assignable grade. For multifocal or diffusely-distributed lesions, this grade was used for processes where less than approximately 10% of the tissue in an average high-power field was involved. For focal or diffuse hyperplastic/hypoplastic/atrophic lesions, this grade was used when the affected structure or tissue had undergone a less than approximately 10% increase or decrease in volume.

Mild. This corresponds to a histopathologic change that is a noticeable but not a prominent feature of the tissue. For multifocal or diffusely-distributed lesions, this grade was used for processes where between approximately 10% and 25% of the tissue in an average high-power field was involved. For focal or diffuse hyperplastic/hypoplastic/atrophic lesions, this grade was used when the affected structure or tissue had undergone between an approximately 10% to 25% increase or decrease in volume.

Moderate. This corresponds to a histopathologic change that is a prominent but not a dominant feature of the tissue. For multifocal or diffusely-distributed lesions, this grade was used for processes where between approximately 25% and 50% of the tissue in an average high-power field was involved. For focal or diffuse hyperplastic/hypoplastic/atrophic lesions, this grade was used when the affected structure or tissue had undergone between an approximately 25% to 50% increase or decrease in volume.

Marked. This corresponds to a histopathologic change that is a dominant but not an overwhelming feature of the tissue. For multifocal or diffusely-distributed lesions, this grade was used for processes where between approximately 50% and 95% of the tissue in an average high-power field was involved. For focal or diffuse hyperplastic/hypoplastic/atrophic lesions, this grade was used when the affected structure or tissue had undergone between an approximately 50% to 95% increase or decrease in volume.

Severe. This corresponds to a histopathologic change that is an overwhelming feature of the tissue. For multifocal or diffusely-distributed lesions, this grade was used for processes where greater than approximately 95% of the tissue in an average high-power field was involved. For focal or diffuse hyperplastic/hypoplastic/atrophic lesions, this grade was used when the affected structure or tissue had undergone a greater than approximately 95% increase of decrease in volume.

Table 30 shows apoptosis, immunoreactivity and inflammation scoring from study day 2. Table 31 shows apoptosis, immunoreactivity and inflammation scoring from study day 4. Table 32 shows apoptosis, immunoreactivity and inflammation scoring from study day 6. Table 33 shows apoptosis, immunoreactivity and inflammation scoring from study day 10.

TABLE 30
Study Day 2

| | PBS | Rx Group |
|---|---|---|
| Groups | (n = 4) | (n = 4) |
| Apoptosis | | |
| Minimal | 3 | — |
| SARS-CoV-2 Immunoreactivity | | |
| Minimal | — | 1 |
| Mild | 1 | 3 |
| Moderate | 3 | — |
| Inflammation, mixed or mononuclear cell | | |
| alv/br/BA/Interstitial | | |
| Minimal | 3 | 3 |
| Mild | 1 | — |
| Inflammation, mononuclear cell, | | |
| vascular/perivascular | | |
| Minimal | — | 1 |
| Mild | — | — |

alv = alveolar,
br = bronchial,
BA = bronchoalveolar

TABLE 31
Study Day 4

| | PBS | Rx Group |
|---|---|---|
| Groups | (n = 4) | (n = 4) |
| Alveolar Hemorrhage | | |
| Minimal | 1 | — |
| Hyperplasia | | |
| Minimal | 1 | — |
| SARS-CoV-2 Immunoreactivity | | |
| Minimal | — | 2 |
| Mild | 2 | 1 |
| Moderate | 2 | — |
| Inflammation, mixed or mononuclear cell | | |
| alv/br/BA/Interstitial | | |
| Minimal | — | 1 |
| Mild | — | 1 |
| Moderate | 4 | — |
| Inflammation, mononuclear cell, | | |
| vascular/perivascular | | |
| Minimal | — | 2 |
| Mild | 4 | — |

alv = alveolar,
br = bronchial,
BA = bronchoalveolar

TABLE 32
Study Day 6

| | PBS | Rx Group |
|---|---|---|
| Groups | (n = 4) | (n = 4) |
| Edema | | |
| Minimal | 3 | 2 |
| Mild | 1 | — |
| Alveolar Hemorrhage | | |
| Minimal | 4 | — |
| Hyperplasia | | |
| Minimal | — | — |
| Mild | 4 | 2 |
| SARS-CoV-2 Immunoreactivity | | |
| Minimal | 1 | 1 |
| Mild | 1 | — |
| Inflammation, mixed or mononuclear cell | | |
| alv/br/BA/Interstitial | | |
| Minimal | — | — |
| Mild | — | 1 |
| Moderate | 3 | 1 |
| Marked | 1 | — |
| Inflammation, mononuclear cell, | | |
| vascular/perivascular | | |
| Minimal | — | — |
| Mild | 4 | 2 |
| Syncytial Cell | | |
| Minimal | 4 | 1 |

alv = alveolar,
br = bronchial,
BA = bronchoalveolar

TABLE 33

| | PBS | Rx Group |
|---|---|---|
| Groups | (n = 4) | (n = 4) |
| Edema | | |
| Minimal | 1 | — |
| Hyperplasia | 4 | — |
| Minimal | — | — |
| Mild | 1 | — |
| Moderate | 3 | — |
| SARS-CoV-2 Immunoreactivity | | |
| Inflammation, mixed or mononuclear cell | | |
| alv/br/BA/Interstitial | | |
| Minimal | 3 | — |
| Mild | — | — |
| Moderate | 1 | — |
| Inflammation, mononuclear cell, | | |
| vascular/perivascular | | |
| Minimal | 2 | — |
| Syncytial Cell | | |
| Minimal | 3 | — |

alv = alveolar,
br = bronchial,
BA = bronchoalveolar

Taken together, these examinations revealed that at all time points, PBS-treated animals had a higher degree of interstitial and bronchoalveolar inflammation and more SARS-CoV-2 nucleocapsid immunoreactivity when compared to antibody-treated animals, suggesting that antibody treatment was effective at treating and clearing BA.5 infection from the lungs.

### Example 13

### SARS-CoV-2 Escape Potential by Multispecific Antibodies

The escape potential of additional multispecific antibodies was assessed using an rcVSV antibody escape assay as described above in Example 5 and by Wang et al., Science, 373:1-15 (2021).

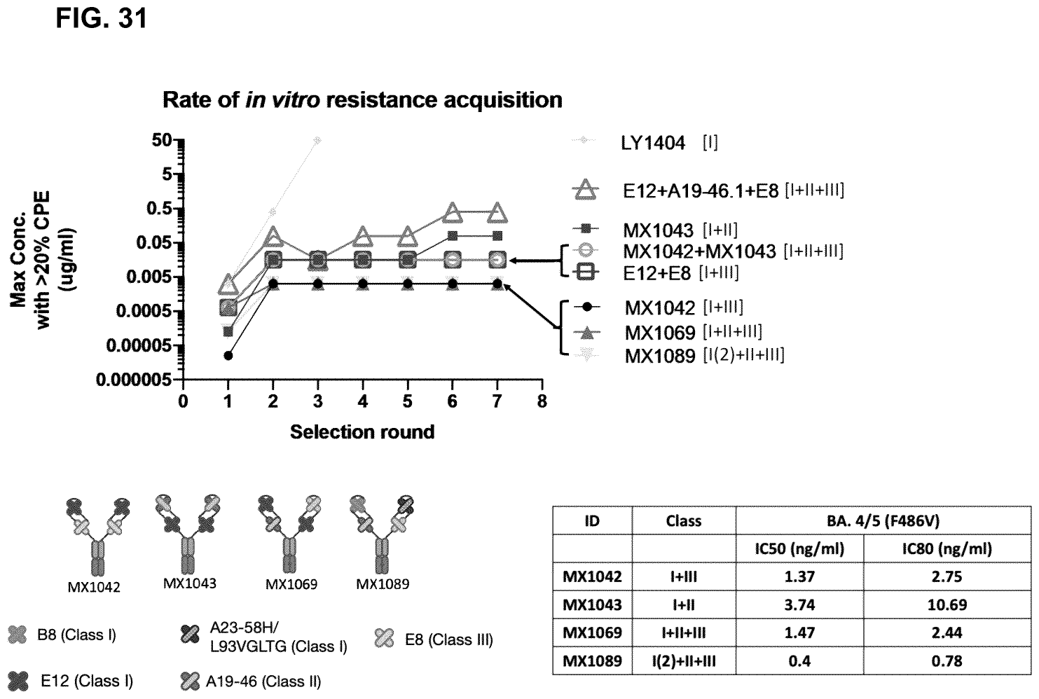

FIG. 31 shows SARS-CoV-2 escape potential by multispecific antibodies MX1043, MX1042, MX1069 and MX1089, compared to combinations of antibodies (E12+A19-46.1+E8; E12+E8; and MX1042+MX1043) and LY1404. Shown for each antibody is the maximum concentration with >20% CPE that was passaged forward in each round of selection. Once significant viral growth appeared, supernatant was no longer passaged forward, and generation of escape variants was considered fixed. Structures for the relevant antibodies and the resulting IC50 and IC80 values are also shown.

These results show that MX1042, MX1069 and MX1089 prevented variant generation at a concentration 25-fold less than MX1043 in 7 rounds of passage.

### Example 14

### Pharmacokinetic Data

A pharmacokinetic study was performed to assess the properties of parental monoclonal antibodies F769-E12, F769-B1, E76-B8 and F770-E8, compared to bispecific antibodies MX1042 (E8×E12) and MX1043 (E12×A19-46); trispecific antibody MX1069 (A19-46×E12/E12×E8); and tetraspecific antibodies MX1089 (A19-46×B8/E8×A23-58H/L93VGLTG) and MX1206 (A19-46×E12/E8×A23-58H/L93VGLTL).

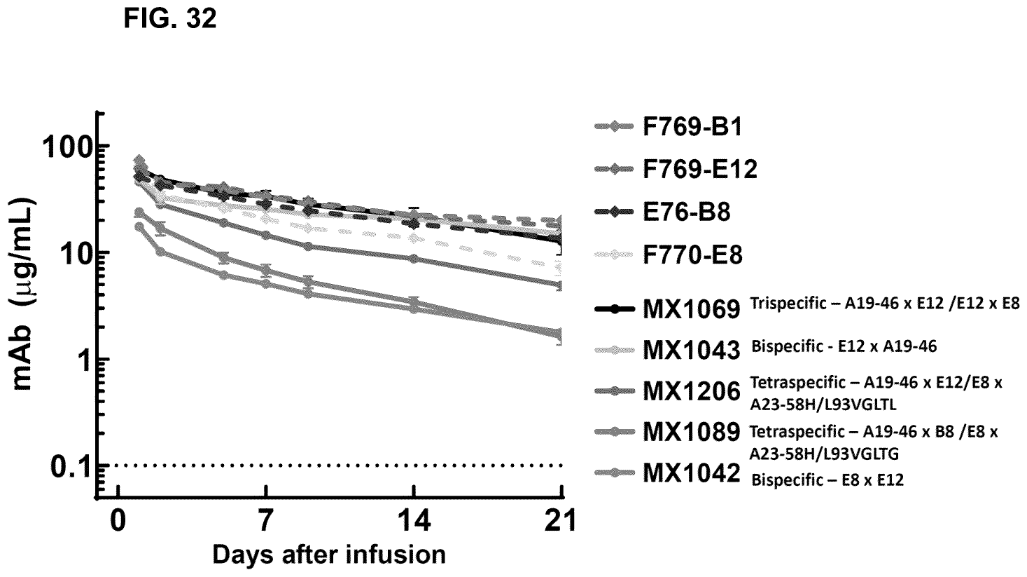

Five human FcRn-Fc mice were used for each antibody, and a single dose of 5 mg/kg was administered intravenously for all antibodies. Sera antibody levels were determined by SARS-CoV-2 S2P-coated ELISA. FIG. 32 shows the average antibody concentration measured up to 21 days after antibody dosing.

### Example 15

### mRNA Synthesis

Protein sequences for MX412 (A23-58H/B1-182L×A19-61), MX413 (A19-61×A23-58H/B1-182L), MX490 (A23-58H/B1-182L×A19-46), and MX492 (A19-46×A23-58H/B1-182L) were back-translated using Benchling to generate the corresponding DNA sequence. This DNA sequence was then cloned into the pUC57-Mini Vector (Genescript), which contains a T7 promoter, a 5′ untranslated region (UTR), and a 3′ UTR. PCR was performed to generate a template for mRNA synthesis, with a 120 nt polyA tail added. In vitro mRNA synthesis was carried out using the HiScribe® T7 High Yield RNA Synthesis Kit (New England Biolabs) from the PCR products. To incorporate modified nucleosides, pseudouridine-5′-Triphosphate (TriLink BioTechnologies; N-1019) was used instead of UTP.

The resulting mRNA was capped using the CleanCap Reagent AG (TriLink BioTechnologies; N-7113) to obtain cap1. Purification of the mRNA was performed using the MEGAclear Transcription Clean-Up Kit (Thermo Fisher Scientific; AM1908). The integrity of the RNAs was confirmed by electrophoresis on agarose gels, and then stored at −80° C. mRNA sequences for MX412, MX413, MX490 and MX492 were as follows: SEQ ID NO:695 (MX412 mRNA with signal peptide), SEQ ID NO:696 (MX412 without signal peptide), SEQ ID NO:697 (MX413 with signal peptide), SEQ ID NO:698 (MX413 without signal peptide), SEQ ID NO:699 (MX490 with signal peptide), SEQ ID NO:700 (MX490 without signal peptide), SEQ ID NO:701 (MX492 with signal peptide) and SEQ ID NO:702 (MX492 without signal peptide).

### Example 16

### mRNA-LNP

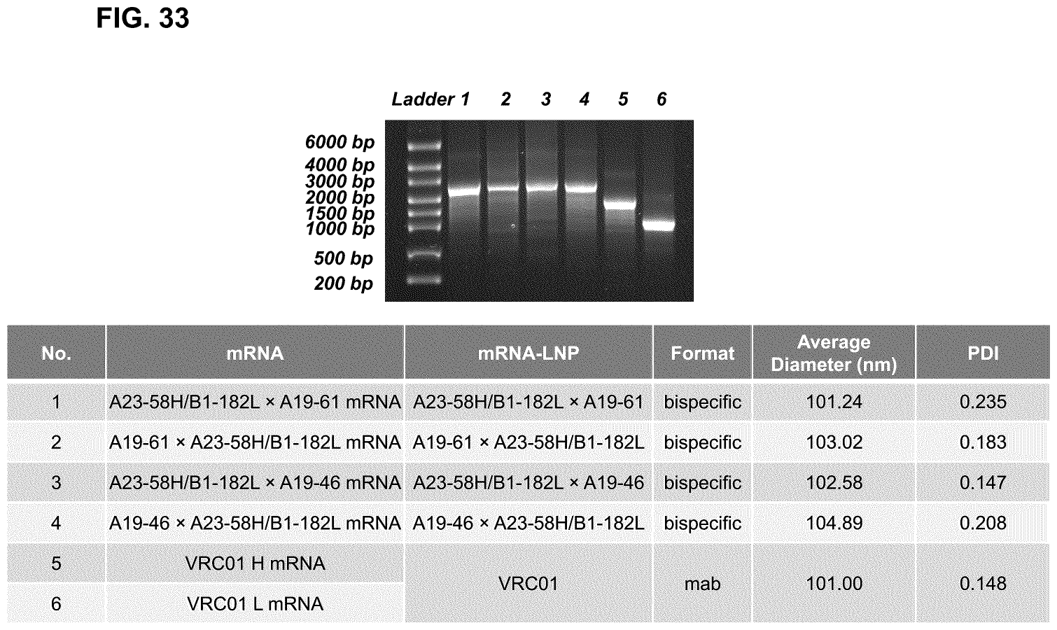

mRNA from Example 15 was incorporated into lipid nanoparticle (LNP) formulations using NanoAssemblr Ignite (Precision Nanosystems) with GenVoy ILM, following the manufacturer's instructions. The formulations were concentrated using Amicon Ultra-15 Centrifugal Filter Units (MilliporeSigma), filtered through a 0.22-μm filter, and stored at 4° C. until further use. Particle size and RNA encapsulation were assessed for all formulations. Results are shown in FIG. 33.

### In Vitro Expression

Expi293 cells were transfected with mRNA using Lipofectamine MessengerMAX Reagent (Thermo Fischer Scientific). The transfected cells were cultured in a shaker incubator at 120 rpm, 37° C., and 9% CO₂ for 5 days. Culture supernatants were harvested, filtered, and the multispecific antibodies were purified using a Protein A (GE Health Science) column. Elution of each multispecific antibody was done using Pierce IgG Elution Buffer, pH 2.0 (Thermo Fisher Scientific), followed by immediate buffer exchange with Histidine pH 6.0 and concentration using Amicon Ultra-15 Centrifugal Filter Units (MilliporeSigma). After concentration, each multispecific antibody was analyzed using reduced and non-reduced sodium dodecyl sulfate-polyacrylamide gel electrophoresis (SDS-PAGE).

### In Vivo Expression

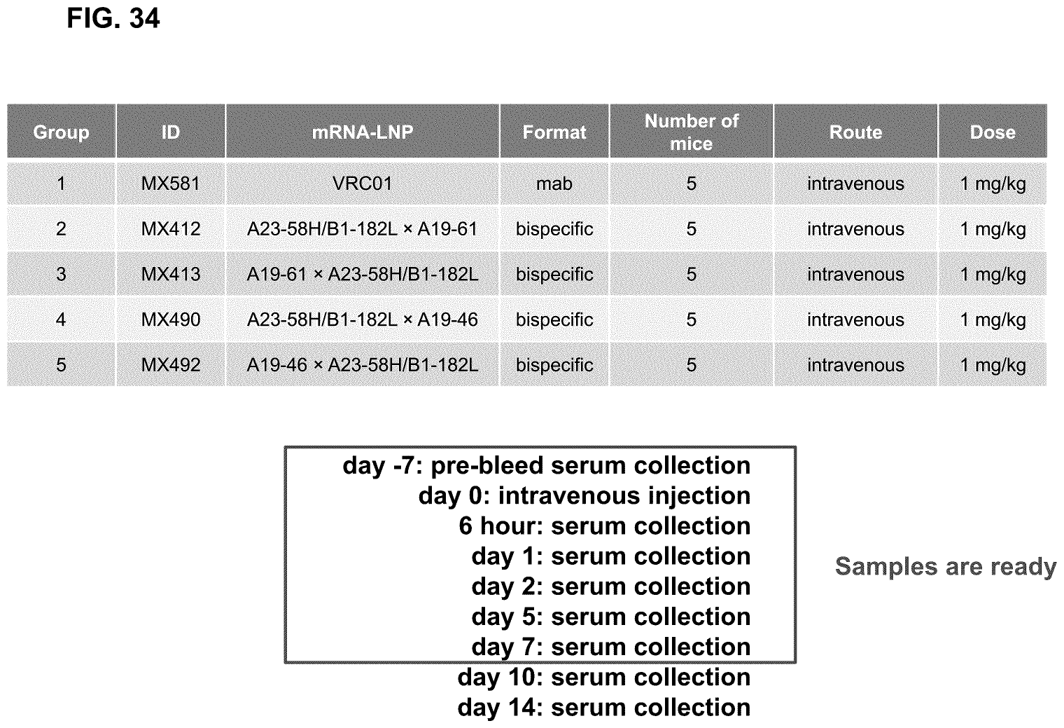

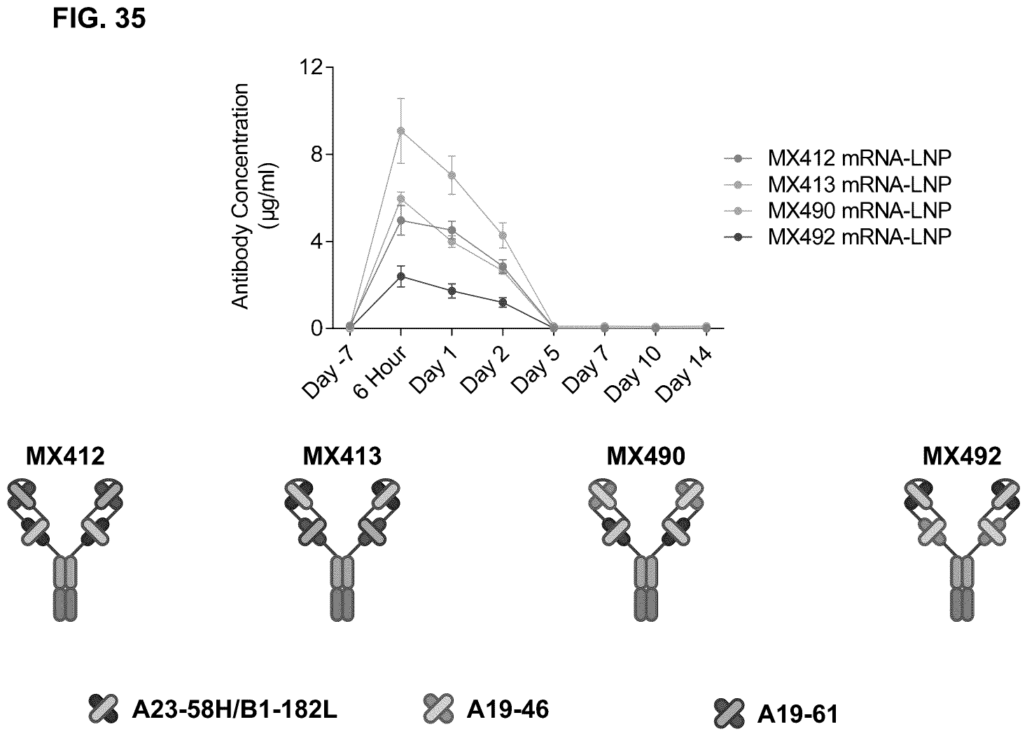

The in vivo expression of mRNA-LNPs was evaluated using a mouse model summarized in FIG. 34. Tg32-hFc mice aged between 6-12 weeks were obtained from The Jackson Laboratory (Bar Harbor, ME). On Day 0, all mice were intravenously injected with a dose of 1 mg/kg of the mRNA-LNP. Blood and serum samples were collected at various time points (Days −7, 0, 6 hours, 1, 2, 5, 7, 10) in ethylenediaminetetraacetic acid (EDTA). Maximum bleeds were performed on Day 14, after which the mice were euthanized. The concentration of the antibody in serum was measured using quantitative ELISA with SARS-CoV-2 spike protein-coated microtiter plates and a horseradish peroxidase (HRP)-conjugated anti-human IgG antibody. Results are shown in FIG. 35.

### Example 17

### Design of Trispecific Antibodies

The trispecific antibody format follows a previously-described design configuration (DOI: 10.1126/science.aan8630). Briefly, a cross-over dual variable domain (CODV)-Ig bispecific antibody arm was heterodimerized with a conventional antibody arm using knob-into-hole (doi:10.1038/nbt0798-677) modifications in the CH3 domain of IgG1 Fc. Specifically, three classes of anti-SARS-CoV-2 neutralizing antibodies targeting spike protein receptor binding domains (RBDs) were used: B1-182.1 (class I), A19-46.1 (class II), and A19-61.1 (class III). These classes were tested in various combinations including each antibody Fv orientation and copy number to achieve bi- or tri-specificity. (As used herein, the term “Fv” represents the antigen binding domain of an antibody containing a variable heavy region and a variable light region.) These combinations were tested for best neutralizing breadth/potency and good manufacturability. In addition, “knob” (S354C/T366W), “hole” (Y349C/T366S/L368A/Y407V), and “LS” (M428L/N434S) (doi: 10.1038/nbt.1601) mutations were engineered into CH3 of the monospecific, bi- or tri-specific Fc region.

### Example 18

### Synthesis, Cloning and Expression of Multispecific Antibodies

After design of the amino acid sequences for each multispecific antibody, the four genes for each multispecific antibody were synthesized using human preferred codons (GenScript) and cloned into eukaryotic expression vectors. For each multispecific antibody expression, equal amounts of the 4 plasmid DNAs were transfected into Expi293 cells (Life Technology) using Expi293 transfection reagent (Life Technology) as previously reported (DOI: 10.1126/science.aan8630). The transfected cells were cultured in a shaker incubator at 120 rpm, 37° C., 9% CO2 for 4˜5 days.

Culture supernatants were harvested and filtered, the multispecific antibodies were purified over a Protein A (GE Health Science) column. Each multispecific antibody was eluted with IgG elution buffer (Pierce), immediately buffer exchanged with phosphate buffered saline (PBS) and concentrated using Centricon Plus-70 (Millipore Sigma) membrane filter unit. After concentration, each multispecific antibody was applied to a Superdex 200 16/600 size exclusion column (Cytiva) to remove aggregates and different species in the preparation. The fractions were then analyzed on reduced and non-reduced SDS-PAGE to identify the fractions that contained the monomeric multispecific antibody before combining them. The pooled fractions were then further concentrated, aliquoted and analyzed by SDS-PAGE as well as an analytical SEC column (Superdex 200 16/600) to verify purity.

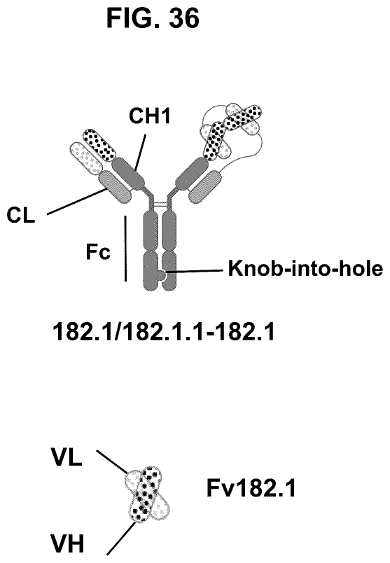

FIG. 36 shows an exemplary configuration of a monospecific, trivalent cross-over dual variable (CODV) immunoglobulin antibody made utilizing variable fragments (Fv) from B1-182.1 (Fv182.1, dotted). The Fc region contains a knob-into-hole modification to increase yield and correct association of heavy chains.

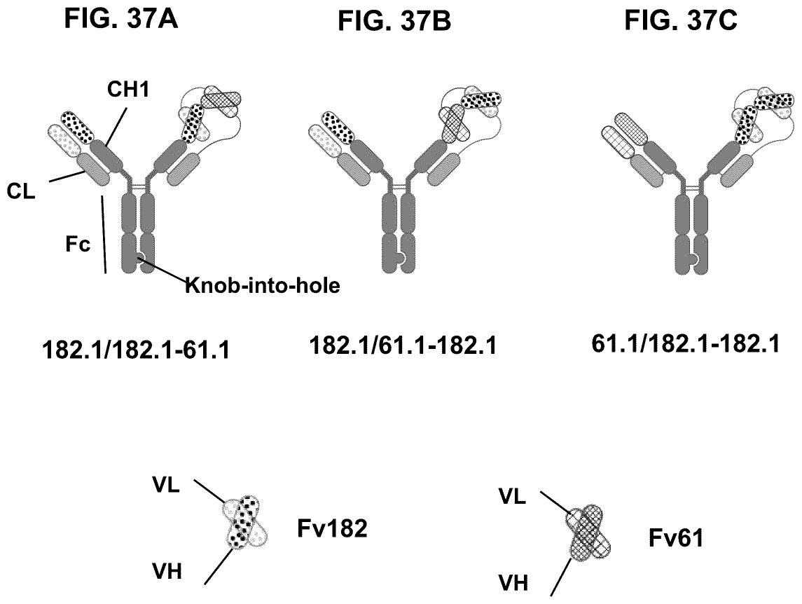

FIG. 37A-37C shows exemplary configurations of bispecific, trivalent CODV immunoglobulin antibodies made utilizing variable fragments (Fv) from B1-182.1 (Fv182.1, dotted) and A19-61.1 (Fv61.1, grid). The Fc region contains a knob-into-hole modification to increase yield and correct association of heavy chains.

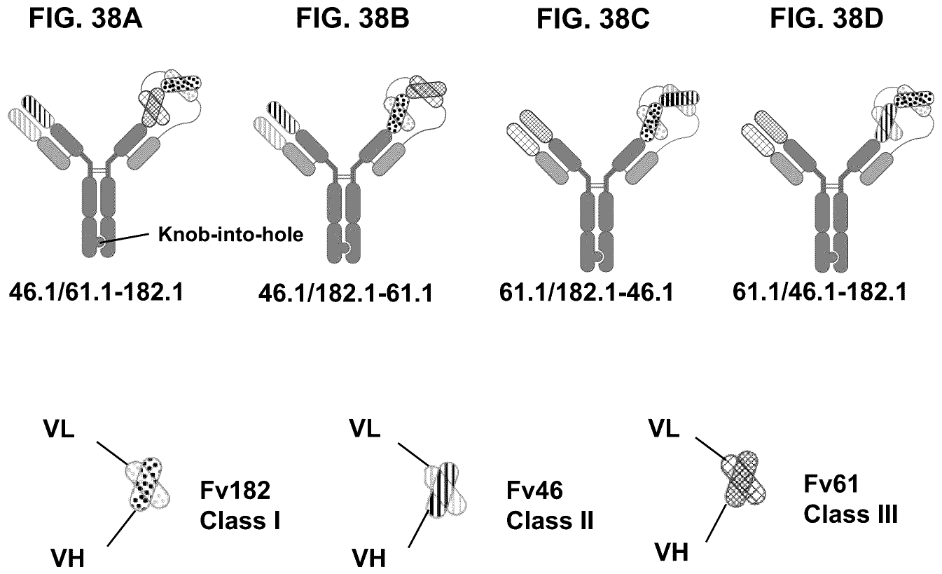

FIG. 38A-38D shows exemplary configurations of trispecific, trivalent CODV immunoglobulin antibodies made utilizing variable fragments (Fv) from B1-182.1 (“182.1” or “Fv182 Class I”, dotted), A19-46.1 (“46.1” or “Fv46 Class II”, striped) and A19-61.1 (“61.1” or “Fy61 Class III”, grid). The Fe region contains a knob-into-hole modification to increase yield and correct association of heavy chains.

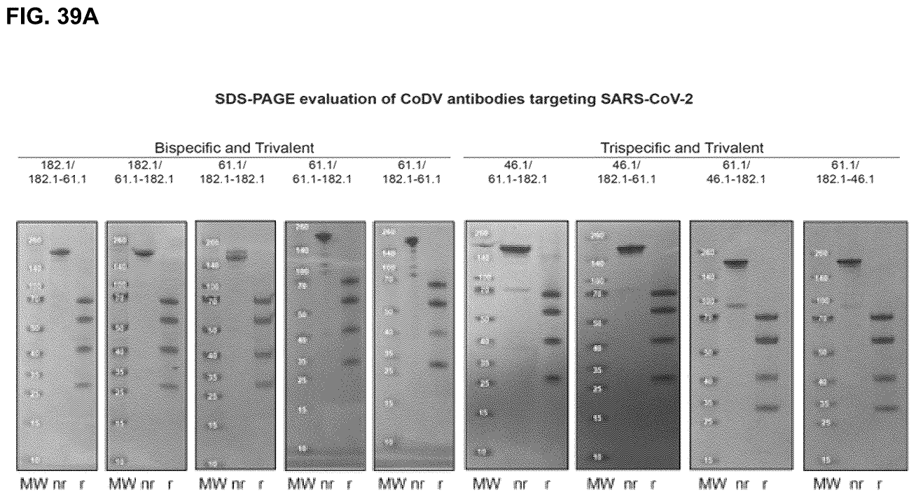

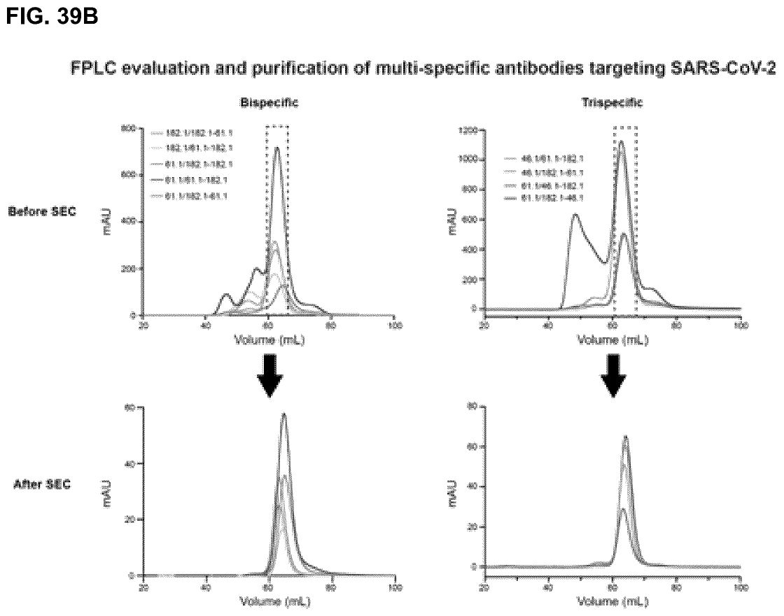

FIG. 39A shows purity of CODV immunoglobulin antibodies evaluated under non-reducing (nr) and reducing (r) conditions on a Coomassie SDS-PAGE gel (representative gels shown). Antibody sequences are shown below in Table 34.

TABLE 34
Sequences

| Antibody | SEQ ID NOs: |
|---|---|
| 182.1/182.1-61.1 | 113, 114, 119-122 |
| 182.1/61.1-182.1 | 93-96, 119-122 |
| 61.1/182.1-182.1 | 97-100, 115-118 |
| 61.1/61.1-182.1 | 97-100, 119-122 |
| 61.1/182.1-61.1 | 97-100, 113-114, 123, 124 |
| 46.1/61.1-182.1 | 87-89, 119-122 |
| 61.1/46.1-182.1 | 97-100, 83-86 |
| 61.1/182.1-46.1 | 97-100, 109-112 |

FIG. 39B shows CODV immunoglobulin bispecific and trispecific antibody traces shown before (top row) and after size exclusion chromatography (SEC) (bottom row). The dotted box indicates the fractions combined to make final preparations of the indicated multispecific antibodies. The bottom row shows analytic SEC traces for the purified multispecific antibodies. Shown are representative traces from production runs of antibodies. Antibody properties and yields are shown in Table 35.

TABLE 35
Biochemical Properties and Production Yields Following
SEC Purification For SARS-CoV-2 CODV Antibodies

| | Molecular | | Extinction | |
|---|---|---|---|---|
| | Weight | Isoelectric | Coefficient. | Yield |
| ID | (kDa) | Point | (M⁻¹ cm⁻¹) | (mg/L) |
| 182.1/182.1-182.1 | 173.1 | 8.38 | 267120 | 14.5 |
| 182.1/182.1-61.1 | 173.4 | 8.39 | 271465 | 3.94 |
| 182.1/61.1-182.1 | 173.4 | 8.39 | 271465 | 2.6 |
| 61.1/182.1-182.1 | 173.4 | 8.39 | 271465 | 4.37 |
| 61.1/61.1-182.1 | 173.7 | 7.81 | 275810 | 1.6 |
| 61.1/182.1-61.1 | 173.7 | 7.81 | 275810 | 9.6 |
| 46.1/61.1-182.1 | 173.7 | 8.32 | 280280 | 9.24 |
| 46.1/182.1-61.1 | 173.7 | 8.32 | 280280 | 16.41 |
| 61.1/46.1-182.1 | 173.7 | 8.32 | 280280 | 7.94 |
| 61.1/182.1-46.1 | 173.7 | 8.32 | 280280 | 17.72 |

### Example 19

### Pseudovirus Neutralization Assay

The pseudotyped neutralization potency (IC50 and IC80) of multispecific antibodies described in Examples 1 and 2 was then determined. Specifically, S-containing lentiviral pseudovirions were produced by co-transfection of packaging plasmid pCMVdR8.2, transducing plasmid pHR′ CMV-Luc, a TMPRSS2 plasmid and S plasmids from SARS-CoV-2 variants into 293T cells using Lipofectamine 3000 transfection reagent (L3000-001, ThermoFisher Scientific, Asheville, NC). 293T-ACE2 cells (provided by Dr. Michael Farzan) were plated into 96-well white/black Isoplates (PerkinElmer, Waltham, MA) at 75,00 cells per well the day before infection of SARS CoV-2 pseudovirus. Serial dilutions of monoclonal antibodies (mAbs) were mixed with titrated pseudovirus, incubated for 45 minutes at 37° C. and added to cells in triplicate. Following 2 h of incubation, wells were replenished with 150 ml of fresh media. Cells were lysed 72 h later, and luciferase activity was measured with Microbeta (Perking Elmer). Percent neutralization and neutralization IC50 and IC80 values were calculated using GraphPad Prism 8.0.2.

Table 36 shows IC50 and IC80 values for neutralization of the SARS-CoV-2 original Wuhan strain (WT), SARS-CoV-2 D614G spike protein mutant (D614G), Alpha variant (B.1.1.7), Beta variant 1 (B.1.35.1v1), Gamma variant (P.1), Delta variants (B.1.617.2 and AY.1), Kappa variant (B.1.617.1), Iota variant (B.1.526), Lambda variant (C.37), Mu variant (B.1.621), C.1.2 variant, and Omicron variants (BA1.1.529, BA.1, BA.2, BA.2.12.1 and BA.4/5). These results show that the antibodies tested potently inhibited many to all variants tested.

TABLE 36

| | WT | | D614G | | B.1.1.7 | | B.1.351.v1 |
|---|---|---|---|---|---|---|---|
| Name | IC50 | IC80 | IC50 | IC80 | IC50 | IC80 | IC50 |
| A23_58.1 | | | | | 0.0329 | 0.1041 | 0.1227 |
| B1-182.1 | 0.0264 | 0.0692 | 0.0171 | 0.0359 | 0.0262 | 0.0844 | 0.0357 |
| 58H_182L | | | | | 0.0174 | 0.0376 | 0.0305 |
| A19-61.1 | 0.1725 | 0.4078 | 0.0550 | 0.1258 | 0.1747 | 0.3251 | 0.0686 |
| A19-46.1 | 0.2633 | 0.7783 | 0.1379 | 0.3893 | 0.1899 | 0.4859 | 0.8074 |
| 58H_182L/58H_182L/58H_182L | 0.0077 | 0.0223 | 0.0076 | 0.0204 | 0.0183 | 0.0398 | 0.0111 |
| 182.1/182.1-61.1 | 0.0321 | 0.0966 | 0.0178 | 0.0447 | 0.0385 | 0.1148 | 0.0358 |
| 182.1/61.1-182.1 | 0.0694 | 0.1891 | 0.0406 | 0.0812 | 0.0918 | 0.2084 | 0.0446 |
| 61.1/182.1-182.1 | 0.2545 | 0.6804 | 0.0495 | 0.1236 | 0.2383 | 0.3381 | 0.1189 |
| 61.1/61.1-182.1 | | | 0.0620 | 0.1181 | | | 0.0586 |
| 61.1/182.1-61.1 | | | 0.0262 | 0.0583 | | | 0.0626 |
| 46.1/61.1-182.1 | 0.2969 | 0.5228 | 0.0881 | 0.1538 | 0.2829 | 0.5068 | 0.1550 |
| 46.1/182.1-61.1 | 0.1473 | 0.3613 | 0.0346 | 0.0769 | 0.1008 | 0.2644 | 0.0384 |
| 61.1/46.1-182.1 | 0.0769 | 0.2880 | 0.0326 | 0.0764 | 0.0784 | 0.2296 | 0.0548 |
| 61.1/182.1-46.1 | 0.3553 | 0.5566 | 0.0930 | 0.1539 | 0.2582 | 0.2992 | 0.1455 |
| 58H_182L/58H_182L-61.1 | | | | | 0.0412 | 0.1176 | 0.0582 |
| 58H_182L/61.1-58H_182L | | | | | 0.0967 | 0.2177 | 0.0635 |
| 46.1/61.1-58H_182L | | | | | 0.3513 | 0.4160 | 0.2423 |
| 61.1/46.1-58H_182L | | | | | 0.1005 | 0.3097 | 0.1059 |

| | B.1.351.v1 | P.1 | | B.1.617.2 | |
|---|---|---|---|---|---|
| Name | IC80 | IC50 | IC80 | IC50 | IC80 |
| A23_58.1 | 0.3389 | 0.0097 | 0.0437 | 0.0133 | 0.0358 |
| B1-182.1 | 0.1390 | 0.0182 | 0.0429 | 0.0099 | 0.0254 |
| 58H_182L | 0.0966 | 0.0116 | 0.0237 | 0.0104 | 0.0205 |
| A19-61.1 | 0.1127 | 0.1352 | 0.2885 | 0.2090 | 0.2917 |
| A19-46.1 | 2.0716 | 0.2600 | 0.7000 | >66.7 | >66.7 |
| 58H_182L/58H_182L/58H_182L | 0.0339 | 0.003468 | 0.010518 | 0.003844 | 0.009797 |
| 182.1/182.1-61.1 | 0.0806 | 0.019834 | 0.064068 | 0.011632 | 0.033888 |
| 182.1/61.1-182.1 | 0.1080 | 0.044369 | 0.117965 | 0.014564 | 0.036509 |
| 61.1/182.1-182.1 | 0.2217 | 0.214617 | 0.537649 | 0.068636 | 0.141241 |
| 61.1/61.1-182.1 | 0.1268 | | | 0.1316 | 0.2455 |
| 61.1/182.1-61.1 | 0.1369 | | | 0.0580 | 0.1217 |
| 46.1/61.1-182.1 | 0.1989 | 0.1999 | 0.4556 | 0.0859 | 0.1828 |
| 46.1/182.1-61.1 | 0.0816 | 0.0284 | 0.0887 | 0.2503 | 0.8762 |
| 61.1/46.1-182.1 | 0.1262 | 0.0451 | 0.1553 | 0.1540 | 0.1955 |
| 61.1/182.1-46.1 | 0.1799 | 0.2072 | 0.4977 | 0.3807 | 0.4716 |
| 58H_182L/58H_182L-61.1 | 0.1363 | 0.020744 | 0.082974 | 0.018507 | 0.056465 |
| 58H_182L/61.1-58H_182L | 0.1450 | 0.056899 | 0.106242 | 0.035644 | 0.062195 |
| 46.1/61.1-58H_182L | 0.3083 | 0.2459 | 0.2713 | 0.1568 | 0.2862 |
| 61.1/46.1-58H_182L | 0.1949 | 0.0455 | 0.1085 | 0.2301 | 0.2747 |

| | B.1.617.1 | | B.1.526 | | C.37 | |
|---|---|---|---|---|---|---|
| Name | IC50 | IC80 | IC50 | IC80 | IC50 | IC80 |
| A23_58.1 | | | | | | |
| B1-182.1 | 0.0261 | 0.0860 | 0.0639 | 0.1831 | 0.0049 | 0.0150 |
| 58H_182L | | | | | | |
| A19-61.1 | 0.0815 | 0.1732 | 0.0418 | 0.0836 | 0.1099 | 0.1865 |
| A19-46.1 | >66.7 | >66.7 | 0.8033 | 1.8820 | >66.7 | >66.7 |
| 58H_182L/58H_182L/58H_182L | | | | | | |
| 182.1/182.1-61.1 | 0.0105 | 0.0311 | 0.0314 | 0.0711 | 0.0083 | 0.0144 |
| 182.1/61.1-182.1 | 0.0142 | 0.0403 | 0.0446 | 0.0660 | 0.0103 | 0.0203 |
| 61.1/182.1-182.1 | 0.0405 | 0.0903 | 0.1146 | 0.1858 | 0.0371 | 0.0629 |
| 61.1/61.1-182.1 | | | | | | |
| 61.1/182.1-61.1 | | | | | | |
| 46.1/61.1-182.1 | 0.1047 | 0.2997 | 0.0694 | 0.1060 | 0.0902 | 0.2529 |
| 46.1/182.1-61.1 | 0.4675 | 0.9759 | 0.0362 | 0.0670 | 0.2156 | 0.6651 |
| 61.1/46.1-182.1 | 0.0753 | 0.1903 | 0.0525 | 0.0854 | 0.0690 | 0.1149 |
| 61.1/182.1-46.1 | 0.2051 | 0.3456 | 0.0808 | 0.1168 | 0.2133 | 0.2801 |
| 58H_182L/58H_182L-61.1 | | | | | | |
| 58H_182L/61.1-58H_182L | | | | | | |
| 46.1/61.1-58H_182L | | | | | | |
| 61.1/46.1-58H_182L | | | | | | |

| | B.1.621 | | C.1.2 | | AY.1 | |
|---|---|---|---|---|---|---|
| Name | IC50 | IC80 | IC50 | IC80 | IC50 | IC80 |
| A23_58.1 | 0.1240 | 0.2600 | 0.0287 | 0.0656 | | |
| B1-182.1 | 0.0695 | 0.2188 | 0.0549 | 0.1096 | 0.0080 | 0.0192 |
| 58H_182L | 0.0402 | 0.1081 | 0.0207 | 0.0348 | | |
| A19-61.1 | 0.2639 | 0.4296 | 8.4254 | | 0.0557 | 0.1207 |
| A19-46.1 | 1.3387 | 3.8125 | 11.6200 | 21.0215 | >66.7 | >66.7 |
| 58H_182L/58H_182L/58H_182L | 0.0201 | 0.0581 | 0.0042 | 0.0098 | 0.0113 | 0.0247 |
| 182.1/182.1-61.1 | 0.0710 | 0.1460 | 0.0381 | 0.0866 | 0.0049 | 0.0138 |
| 182.1/61.1-182.1 | 0.1122 | 0.2471 | 0.0794 | 0.0944 | 0.0099 | 0.0154 |
| 61.1/182.1-182.1 | 0.1947 | 0.3113 | | | 0.0412 | 0.0594 |
| 61.1/61.1-182.1 | | | | | | |
| 61.1/182.1-61.1 | | | | | | |
| 46.1/61.1-182.1 | 0.2226 | 0.3168 | 0.1148 | 0.2665 | 0.0441 | 0.1220 |
| 46.1/182.1-61.1 | 0.0389 | 0.1157 | | | 0.0882 | 0.4172 |
| 61.1/46.1-182.1 | 0.1559 | 0.3001 | 0.0654 | 0.1943 | 0.0438 | 0.0845 |
| 61.1/182.1-46.1 | 0.2716 | 0.4677 | | | 0.2629 | 0.2880 |
| 58H_182L/58H_182L-61.1 | 0.0966 | 0.1854 | 0.0512 | 0.0650 | | |
| 58H_182L/61.1-58H_182L | 0.2409 | 0.2656 | 0.0071 | 0.0194 | | |
| 46.1/61.1-58H_182L | 0.2862 | 0.3135 | 0.2185 | 0.4543 | | |
| 61.1/46.1-58H_182L | 0.2698 | 0.2858 | 0.0681 | 0.0878 | | |

| | BA1.1.529 | | BA.1 | | BA.2 | | BA.2.12.1 | | BA.4/5 | |
|---|---|---|---|---|---|---|---|---|---|---|
| Name | IC50 | IC80 | IC50 | IC80 | IC50 | IC80 | IC50 | IC80 | IC50 | IC80 |
| A23_58.1 | | | 0.8529 | 4.2362 | 1.7240 | 6.1249 | 0.6471 | 4.9635 | >66.7 | >66.7 |
| B1-182.1 | 2.5540 | 6.4690 | 1.0322 | 4.4388 | 2.1151 | 8.5007 | 0.7393 | 5.9249 | >66.7 | >66.7 |
| 58H_182L | | | 0.3638 | 1.3930 | 0.6631 | 2.8936 | 0.8298 | 3.2735 | >66.7 | >66.7 |
| A19-61.1 | >66.7 | >66.7 | >66.7 | >66.7 | 0.0607 | 0.1699 | 0.1104 | 0.2980 | 0.1282 | 0.3220 |
| A19-46.1 | 1.8360 | 2.8400 | 0.9580 | 2.2810 | 0.6896 | 2.2272 | >66.7 | >66.7 | >66.7 | >66.7 |
| 58H_182L/58H_182L/58H_182L | | | 3.9134 | 24.2954 | 2.3442 | 25.0890 | 4.2697 | >66.7 | >66.7 | >66.7 |
| 182.1/182.1-61.1 | 47.2166 | | 27.2608 | 38.1161 | 0.0632 | 0.1724 | 0.1446 | 0.5118 | 10.0891 | 19.6789 |
| 182.1/61.1-182.1 | 1.2104 | | 1.0695 | 8.1047 | 0.0160 | 0.0626 | 0.0381 | 0.0788 | 2.4939 | 6.9402 |
| 61.1/61.1-182.1 | >66.7 | >66.7 | 0.0677 | 0.1171 | 0.0698 | 0.2894 | 0.0973 | 0.2350 | >66.7 | >66.7 |
| 61.1/182.1-61.1 | >66.7 | >66.7 | 0.0415 | 0.0962 | 0.0507 | 0.1275 | 0.0427 | 0.2009 | >66.7 | >66.7 |
| 61.1/182.1-182.1 | >66.7 | | >66.7 | >66.7 | 0.0859 | 0.2015 | 0.3427 | 0.6587 | 1.8459 | 6.0570 |
| 46.1/61.1-182.1 | 0.8634 | | 0.7366 | 1.8668 | 0.0400 | 0.0900 | 0.3527 | 1.1248 | 11.2895 | 24.6235 |
| 46.1/182.1-61.1 | 1.8580 | | 1.4133 | 3.9563 | 0.0448 | 0.1123 | 1.3441 | 2.4544 | 15.4292 | 36.9370 |
| 61.1/46.1-182.1 | 0.1666 | | 0.1982 | 0.4505 | 0.0255 | 0.0683 | 0.0488 | 0.1064 | 1.2141 | 2.3808 |
| 61.1/182.1-46.1 | 8.3726 | | 5.0902 | 23.0975 | 0.0195 | 0.0663 | 0.3086 | 0.6151 | 1.9607 | 4.4528 |
| 58H_182L/58H_182L-61.1 | | | 22.1760 | 48.0214 | 0.1301 | 0.4851 | 0.6181 | 1.2993 | 16.1758 | 47.7654 |
| 58H_182L/61.1-58H_182L | | | 2.7327 | 11.6180 | 0.0300 | 0.0960 | 0.0532 | 0.1416 | 2.3381 | 6.5572 |
| 46.1/61.1-58H_182L | | | 1.3652 | 3.0719 | 0.0283 | 0.1451 | 0.3664 | 0.7078 | 8.1859 | 19.2642 |
| 61.1/46.1-58H_182L | | | 0.2016 | 0.6393 | 0.0206 | 0.1003 | 0.1193 | 0.2270 | 2.1978 | 6.3612 |

### Example 20

### Validation of Antibody Binding to RBD Mutation Proteins by ELISA and Neutralization

Binding of multispecific antibodies of Examples 17 and 18 to viral spike receptor-binding domain (RBD) mutations was then validated. The RBD mutations that were used contained one or more of the previously-identified knockout mutations for Fv182 (F486S), Fv46 (L452R) and Fv61 (K444E). For the validation assay, MaxiSorp™ Immuno Plates (Thermo Fisher Scientific) were coated with 1 μg/mL of SARS-CoV-2 WA-1 RBD or RBD with single, double or triple mutations (F486S, E444K, L452R, F486S/E444K, F486S/L452R, and F486S/E444K/L452R) in PBS at 4° C. overnight. After standard washes and blocking, plates were incubated with serial dilutions of antibody for one hour at room temperature. Anti-human IgG Fc gamma-specific horseradish peroxidase conjugate (Jackson Laboratory) was used to detect binding of antibody to the RBD proteins. The plates were then washed and developed with 3,5,3′5′-tetramethylbenzidine (TMB) (KPL, Gaithersburg, MD). After stopping the reaction with 1N H₂SO₄ (Fisher), optical density (OD) at 450 nM was read with a SpectraMax® Plus microplate reader (Molecular Devices).

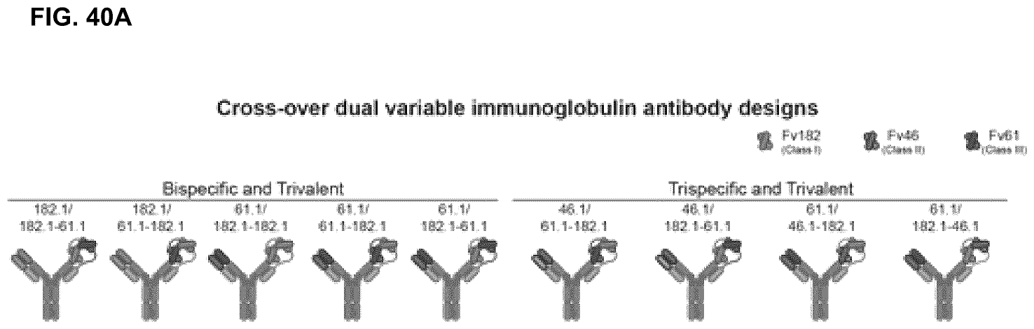

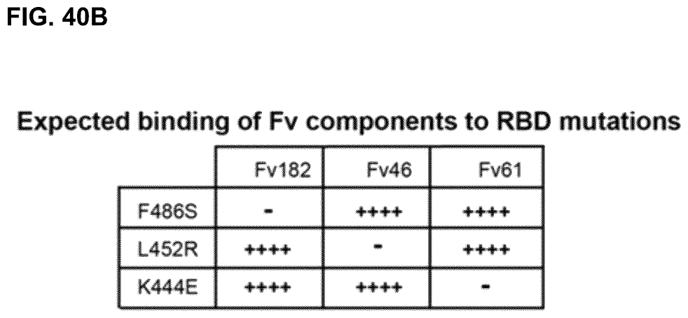

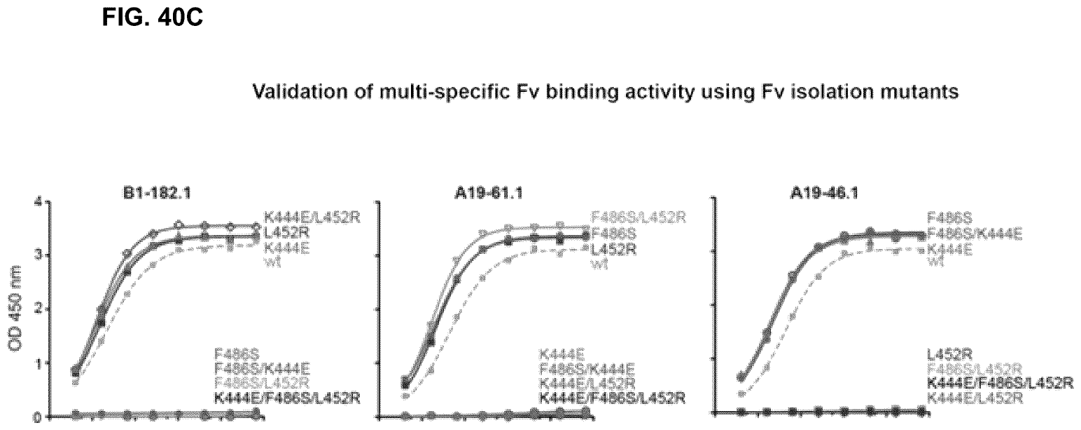

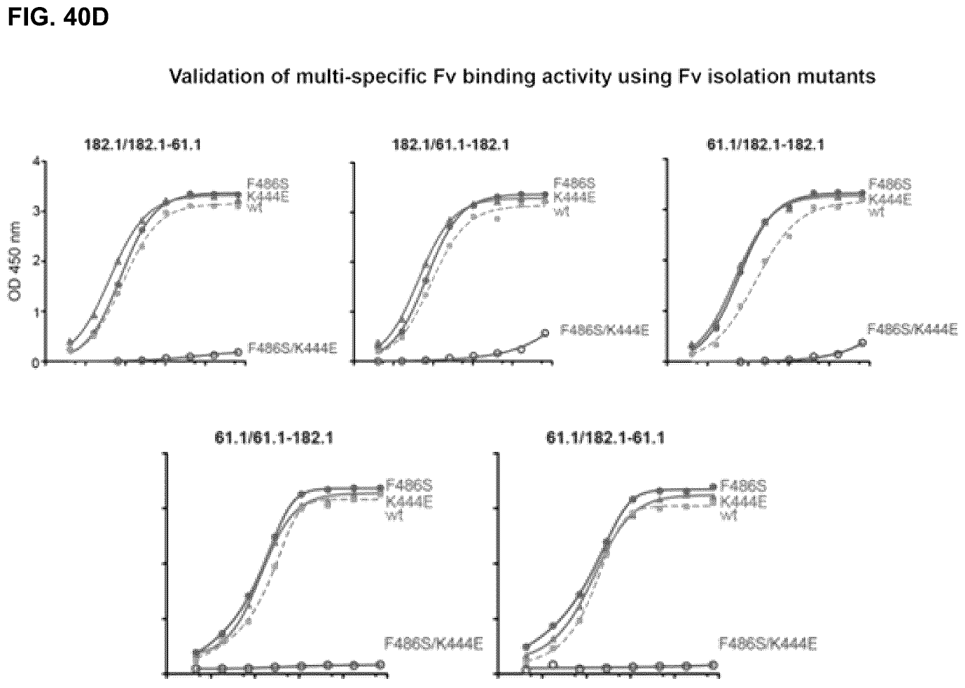

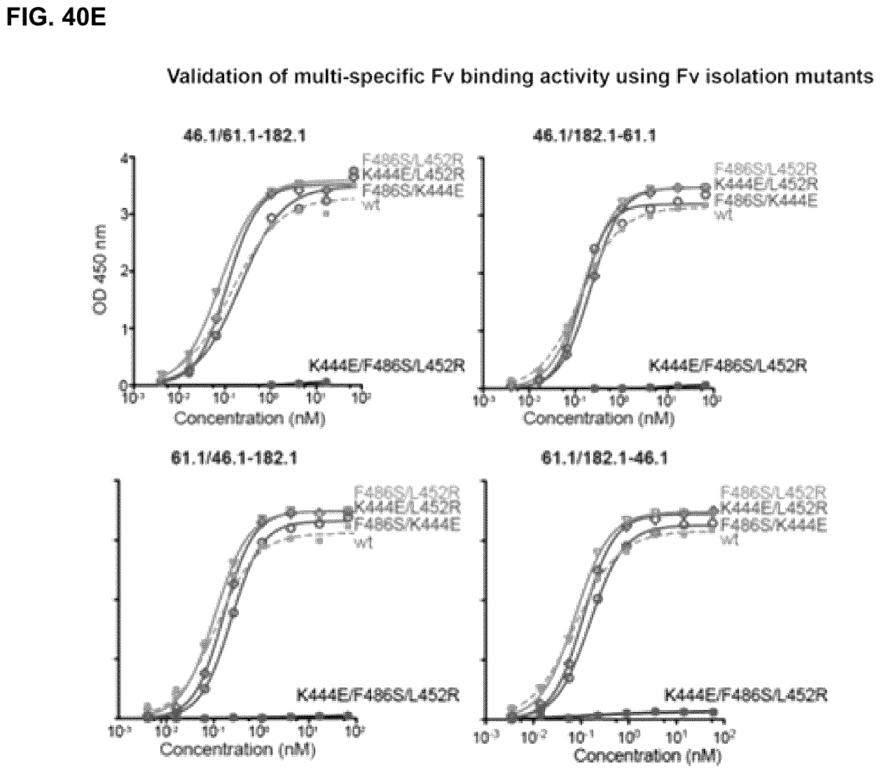

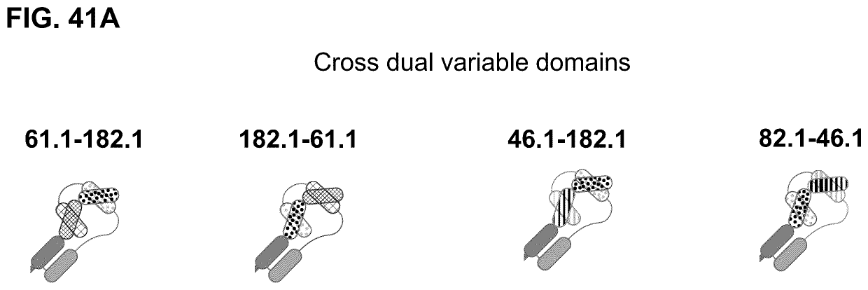

FIG. 40A shows cross-over the dual variable immunoglobulin antibody designs used in this example.

FIG. 40B shows the expected binding of Fv182.1 (“Fv182”), Fv46.1 (“Fv46”), and Fv61.1 (“Fv61”) to RBD proteins containing the indicated Fv isolating mutations that were selected to knockout binding a single component Fv, while leaving binding of the remain Fv components unchanged.

FIG. 40C-40E shows validation of multispecific Fv binding activity using Fv isolating mutations in RBD and enzyme-linked immunosorbent assay (ELISA). Shown is ELISA binding for parental component antibody controls (FIG. 40C, B1-182.1, A19-46.1, and A19-61.1), bispecific antibodies (FIG. 40D, 182.1/182.1-61.1, 182.1/61.1-182.1, 61.1/182.1-182.1, 61.1/61.1-182.1 and 61.1/182.1-61.1), and trispecific antibodies (FIG. 40E, 46.1/61.1-182.1, 46.1/182.1-61.1, 61.1/46.1-182.1 and 61.1/182.1-46.1) against WA-1 RBD (wt) or RBD proteins containing one or more Fv-specific mutations shown and chosen to interrogate binding of one Fv component at a time in each multispecific.

These results show that the candidate multispecific antibodies had the expected binding properties. For example, F486S/K444E showed binding to the Fv46 component of the trispecific antibodies because it does not allow binding of Fv182 and Fv61. And, Fv domains in each multispecific antibody recognized the unmutated epitopes similarly to wild type RBD.

For further validation, an assay was performed to test the ability of each antibody to neutralize spike protein point mutants that eliminated binding of one component and showed that each multispecific maintained neutralization. Table 37 shows the neutralization of candidate multispecific antibodies against D614G or mutants that target Fv182 (“B1-182.1 Escape”; F486S/D614G), Fv46 (“A19-46.1 Escape”; L452R/D614G) and Fv46 (“A19-46.1 Escape”; K444E/D614G).

TABLE 37

| | | | | B1-182.1 Escape | | A19-46.1 Escape | | A19-46.1 Escape | |
|---|---|---|---|---|---|---|---|---|---|
| | | D614G | | F486S/D614G | | L452R/D614G | | K444E/D614G | |
| | | IC₅₀ | IC₈₀ | IC₅₀ | IC₈₀ | IC₅₀ | IC₈₀ | IC₅₀ | IC₈₀ |
| | B1-182.1 | 15.7 | 34.9 | >67,000 | >67,000 | 25.0 | 75.8 | 11.1 | 29.9 |
| | A19-46.1 | 44.1 | 96.5 | 75.2 | 165.8 | 206.9 | 444.2 | >67,000 | >67,000 |
| | A19-61.1 | 113.3 | 339.2 | 132.9 | 390.0 | >67,000 | >67,000 | 472.1 | 1504 |
| Bispecific | 182.1/182.1-61.1 | 11.4 | 36.5 | 403.8 | 1320 | 24.1 | 112.6 | 7.0 | 56.1 |
| and | 182.1/61.1-182.1 | 15.8 | 46.8 | 354.1 | 789.6 | 38.5 | 65.2 | 10.1 | 35.8 |
| Trivalent | 61.1/182.1-182.1 | 67.4 | 128.4 | 320.4 | 1008 | 145.7 | 311.6 | 260.6 | 1090 |
| | 61.1/61.1-182.1 | n.t. | n.t. | n.t. | n.t. | n.t. | n.t. | n.t. | n.t. |
| | 61.1/182.1-61.1 | n.t. | n.t. | n.t. | n.t. | n.t. | n.t. | n.t. | n.t. |
| Trispecific | 46.1/61.1-182.1 | 48.7 | 119.2 | 112.1 | 243.2 | 201.2 | 602.8 | 189.1 | 487.4 |
| and | 46.1/182.1-61.1 | 31.7 | 62.9 | 80.0 | 270.8 | 278.7 | 909.9 | 46.9 | 140.5 |
| Trivalent | 61.1/46.1-182.1 | 30.8 | 48.4 | 68.6 | 163.9 | 232.8 | 934.0 | 42.0 | 139.9 |
| | 61.1/182.1-46.1 | 82.0 | 183.3 | 82.6 | 212.3 | 1010 | 2097 | 89.3 | 356.1 |

Taken together, these findings verified the Fvs within each candidate multispecific antibody functioned as expected.

### Example 21

### Identification of Potential Binding Modes of CODV

### to SARS-CoV-2 Spike Protein by Negative Stain-Electron Microscopy

Next, potential binding modes of CODV to SARS-CoV-2 spike protein were identified by negative stain-electron microscopy. For negative stain-electron microscopy, SARS-CoV-2 spike proteins were mixed with CODV or Fab at a molar ratio of 1:1.2, incubated at room temperature for 10 minutes, and diluted to a concentration of approximately 0.02 mg spike protein/mL with 10 mM HEPES, pH 7.4, 150 mM NaCl. To make a grid, 4.8 μL of the diluted sample was placed on a freshly glow-discharged carbon-coated copper grid for 15 seconds. The drop on the grid was then wicked away with filter paper, and the grid was washed and wicked three times. The same volume of 0.75% uranyl formate was added to the grid to negatively stain protein molecules adsorbed to the carbon and immediately wicked away. After staining three times, the grid was allowed to air-dry. Datasets were collected using a Thermo Scientific™ Talos™ F200C transmission electron microscope equipped with a Ceta camera at 200 kV. The nominal magnification was 57,000×, corresponding to a pixel size of 2.53 Å, and the defocus was set at −2 μm. Data was collected automatically using EPU. Single particle analysis was performed using CryoSPARC 3.0.

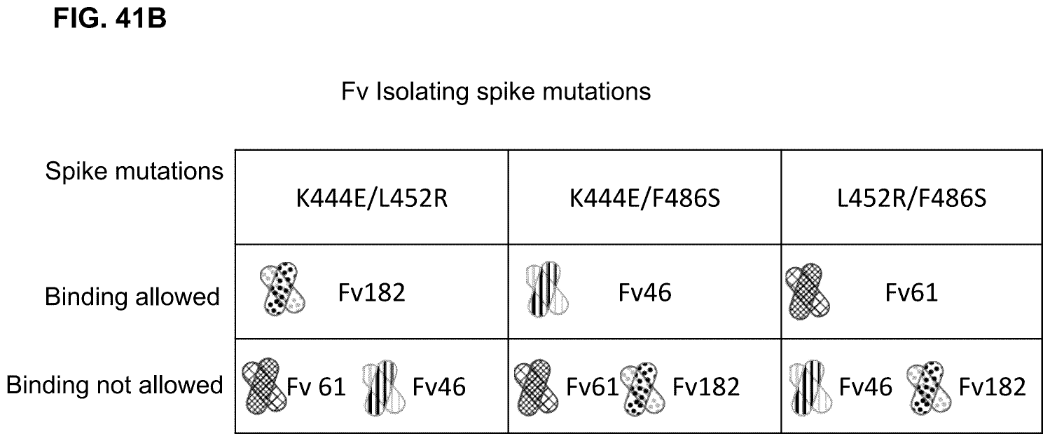

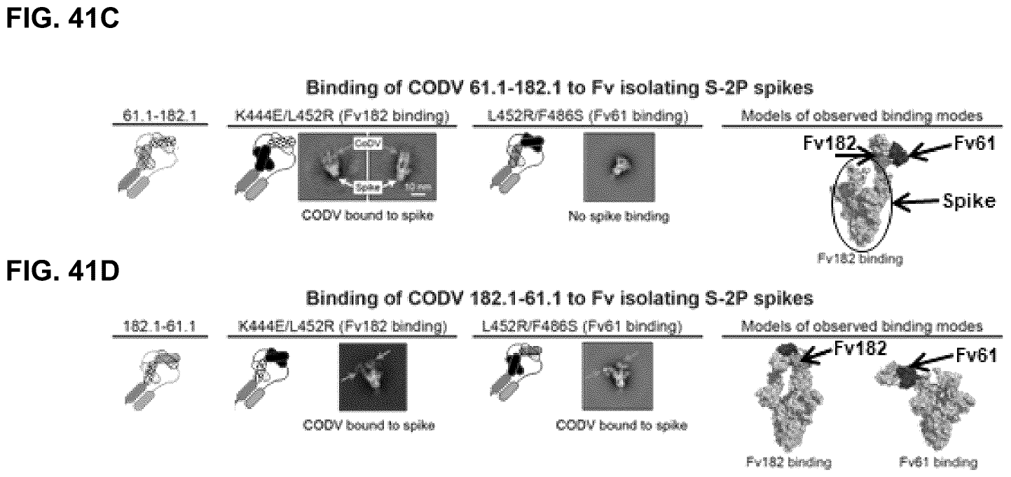

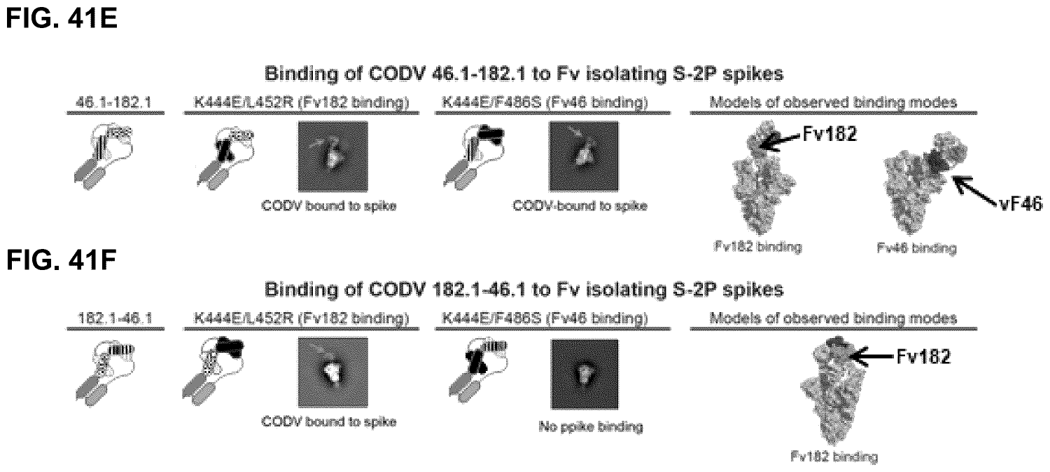

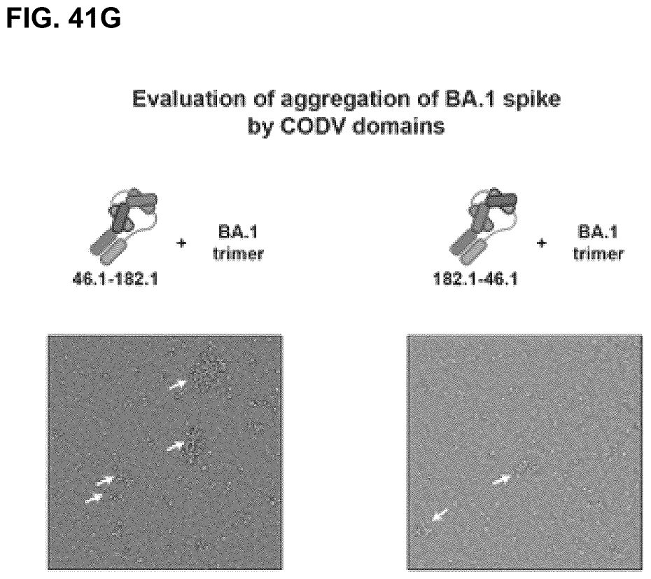

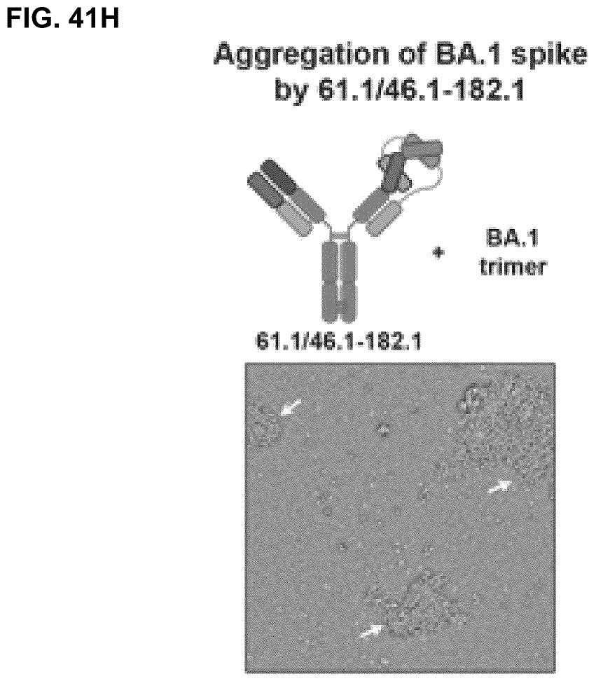

FIG. 41A shows a schematic illustration of arrangement of antibody variable domains in different CODVs. The domains are dotted, striped, grid, and gray for Fv182.1 (182.1), Fv46.1 (46.1), Fv61.1 (61.1) and constant domains, respectively.

FIG. 41B shows combinations of RBD mutations on the SARS-CoV-2 spike protein designed to distinguish different binding modes of CODV.

FIG. 41C is a schematic of CODV 61.1-182.1 (left) and binding of the CODV 61.1-182.1 to spike protein mutants (middle left and right). For clarity, the Fv domain that is not able to bind to the mutant is colored black. CODV binding to K444E/L452R mutant was observed, indicating CODV 61.1-182.1 can bind to spike protein through Fv182 domain (middle left). However, no CODV was observed to bind the L452R/F486S mutant (middle right), suggesting that the CODV 61.1-182.1 cannot bind through the Fv61 domain in the trimer context. A model of the CODV 61.1-182.1 bound to the SARS-CoV-2 spike protein is shown in the allowed Fv182-binding mode (right). For clarity, only one CODV is shown to bind to the spike protein in RBD-up conformation. The white scale bar represents 10 nm.

FIG. 41D is a schematic of CODV 182.1-61.1 (left) and binding of the CODV 182.1-61.1 to spike protein mutants (middle left and right). For clarity, the Fv domain that is not able to bind to the mutant is colored black. CODV was observed to bind both K444E/L452R and L452R/F486S mutants, indicating the CODV 182.1-61.1 can bind to spike protein through either Fv182 domain (middle left) or Fv61 domain (middle right). Models of the CODV 182.1-61.1 bound to the SARS-CoV-2 spike protein are shown in both Fv182.1-binding and Fv61.1-binding modes (right). The scale is the same as in FIG. 41C.

FIG. 41E is a schematic of CODV 46.1-182.1 (left) and binding of the CODV 46.1-182.1 to spike protein mutants (middle left and right). For clarity, the Fv domain that is not able to bind to the mutant is colored black. CODV was observed to bind both K444E/L452R and K444E/F486S mutants, indicating the CODV 46.1-182.1 can bind to the spike protein through both Fv182 and Fv46 domains (middle left and right). Models of the CODV 46.1-182.1 bound to the SARS-CoV-2 spike protein are shown to bind through Fv182 and Fv46-binding modes (right). The scale is the same as in FIG. 41C.

FIG. 41F is a schematic of CODV 182.1-46.1 (left) and binding of the CODV 182.1-46.1 to spike protein mutants (middle left and right). For clarity, the Fv domain that is not able to bind to the mutant is colored black. CODV was observed to bind K444E/L452R, indicating the CODV 182.1-46.1 can bind to the spike protein through the Fv182.1 domain (middle left). However, no CODV was observed to bind the K444E/F486S mutant (middle right), suggesting that the CODV 182.1-46.1 cannot bind through the Fv46 domain in the trimer context. Model of the CODV 182.1-46.1 bound to the SARS-CoV-2 spike protein is shown to bind through the allowed Fv182 domain (right). The scale is the same as in FIG. 41C.

FIG. 41G shows CODV 46.1-182.1- and 182.1-46.1-induced aggregation of the Omicron spike protein. NSEM micrographs revealed that under conditions when CODV 182.1-46.1 only formed small complexes with BA.1 spike trimers, CODV 46.1-182.1 formed large aggregates. This suggests that CODV 46.1-182.1 can efficiently promote inter-spike crosslinking.

While the monovalent 61.1 arm of 61.1/46.1-182.1 is prevented from binding to the Omicron BA.1 spike due to mutations, it is possible that its presence in the full immunoglobulin might impact the ability of the CODV 46.1-182.1 arm to aggregate spikes. The spike-aggregating potential of 61.1/46.1-182.1 using NSEM and noted large amounts of aggregation (FIG. 41H). Taken together, these results illustrate how in the context of spike trimers, both the location of epitopes within RBD and CODV position can impact binding, aggregative potential and neutralization. Specifically, Fvs with an angle of approach vertical to the trimer apex such as Fv182 are likely to have greater flexibility for positioning with trivalent mAb designs, likely due to freedom from steric constraints, and consistent with higher potency neutralization by this Fv. In contrast, Fv46 has a lateral or angled approach suggesting that antibodies that bind with this angle of approach may be subject to steric constraints that are revealed by position-dependence of the Fv for optimal engagement and neutralization.

### Example 22

### SARS-CoV-2 Escape Potential by Single Antibodies, Combinations and Multispecific Antibodies

The escape potential of the candidate multispecific antibodies was assessed using an rcVSV antibody escape assay. Selection of virus escape variants was conducted as previously described by Wang et al., Science, 373:1-15 (2021). Briefly, an equal volume of clonal population of replication competent vesicular stomatitis virus (rcVSV) with its native glycoprotein replaced by the Wuhan-1 spike protein (rcVSV SARS CoV-2) at an MOI of 0.01 was mixed with serial dilutions of antibodies (5-fold) in cell media to give the desired final antibody concentration. Antibody cocktails were mixed at equal ratios. Virus:antibody mixtures were incubated at 37° C. for 1 hour prior to being added to Vero E6 cells. Virus replication was assessed 72 hours after infection in the presence of selected antibodies. Supernatants from well with the highest concentration of antibody which showed evidence of viral replication (>20% cytopathic effect) was passaged into the subsequent rounds of selection. Infection, monitoring, and collection of supernatants was performed as in the initial round.

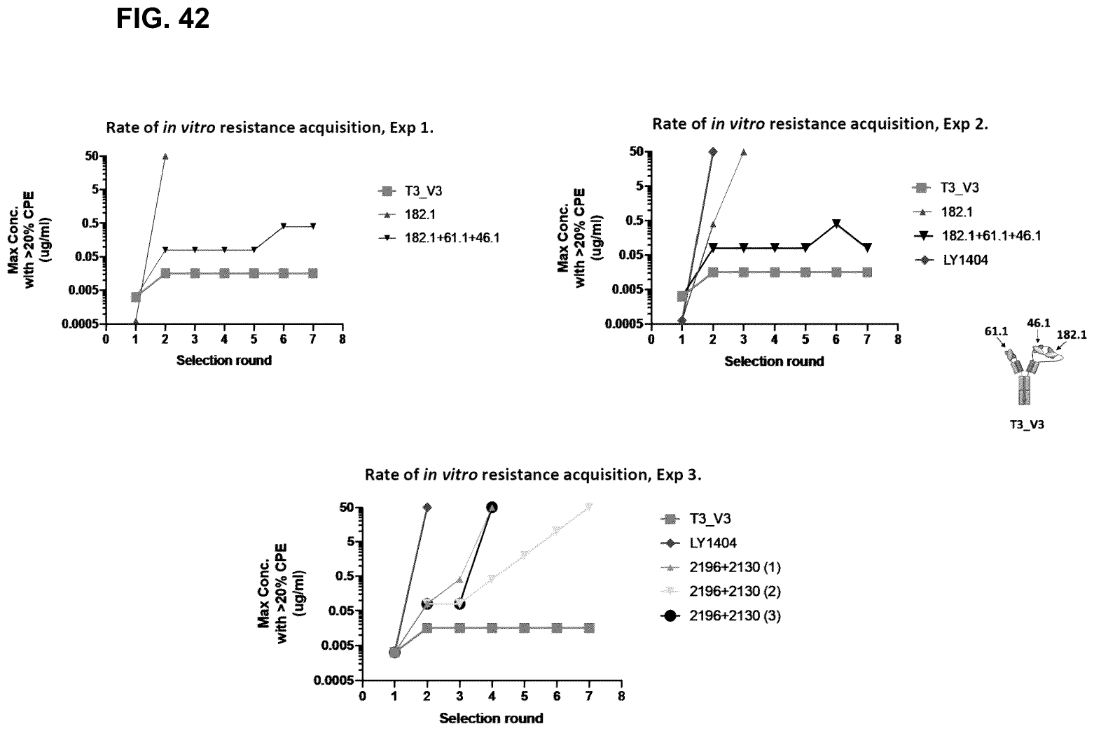

FIG. 42 shows results from three independent experiments showing SARS-CoV-2 escape potential of T3_V3 (having 61.1, 46.1 and 182.1 binding regions as indicated in FIG. 42), compared to an antibody having binding regions for 182.1, 61.1 and 46.1 (182.1+61.1+46.1); an antibody having binding regions for 182.1 (182.1); bebtelovimab (LY1404); and an AZD7442 cocktail (2196+2130). T3_V3 was better at preventing the acquisition of resistance than 182.1, LY1404 and AZD7442. In addition, the maximum concentration at which there was a loss of neutralization was 0.016 μg/mL for T3_V3 in all three experiments.

### Example 23

### E8 mAb Glycosylation Mutations

N62Q and N62S mutations were made in the E8 heavy chain variable domain (VH) sequence, which remove a high mannose glycosylate residue or moiety. The specific sequences prepared are listed in Table 38.

TABLE 38

| SEQ ID NO: | Description |
|---|---|
| 279 | E8 VH wild type (N62) - peptide |
| 778 | E8 VH wild type (N62) - DNA |
| 779 | E8 VH N62Q - peptide |
| 780 | E8 VH N62Q - DNA |
| 781 | E8 VH N62S - peptide |
| 782 | E8 VH N62S - DNA |
| 785 | E8 VH N62Q in expression plasmid - peptide |
| 786 | E8 VH N62Q in expression plasmid - DNA |
| 787 | E8 VH N62S in expression plasmid - peptide |
| 788 | E8 VH N62S in expression plasmid - DNA |

The E8 VL sequence of SEQ ID NO:275 was used for preparing the antibodies (corresponding to SEQ ID NO:783 (E8 VL in expression vector—peptide) and SEQ ID NO:784 (E8 VL in expression plasmid—DNA)).

Human FcRn transgenic mice (C57BL/6, B6.mFcRn−/−hFcRn Tg32 line from The Jackson Laboratory) were used to assess the pharmacokinetics of the antibodies. Each animal was infused intravenously with 5 mg antibody/kg of body weight. Whole blood samples were collected at days 1, 2, 5, 7, 9, 14, 21, 28, and 35. Serum was separated by centrifugation. Serum antibody levels were measured by ELISA. All mice were bred and maintained under pathogen-free conditions at an American Association for Assessment and Accreditation of Laboratory Animal Care International-accredited animal facility at the National Institute of Allergy and Infectious Diseases and housed in accordance with the procedures outlined in the Guide for the Care and Use of Laboratory Animals. All mice were between 6 and 13 weeks of age.

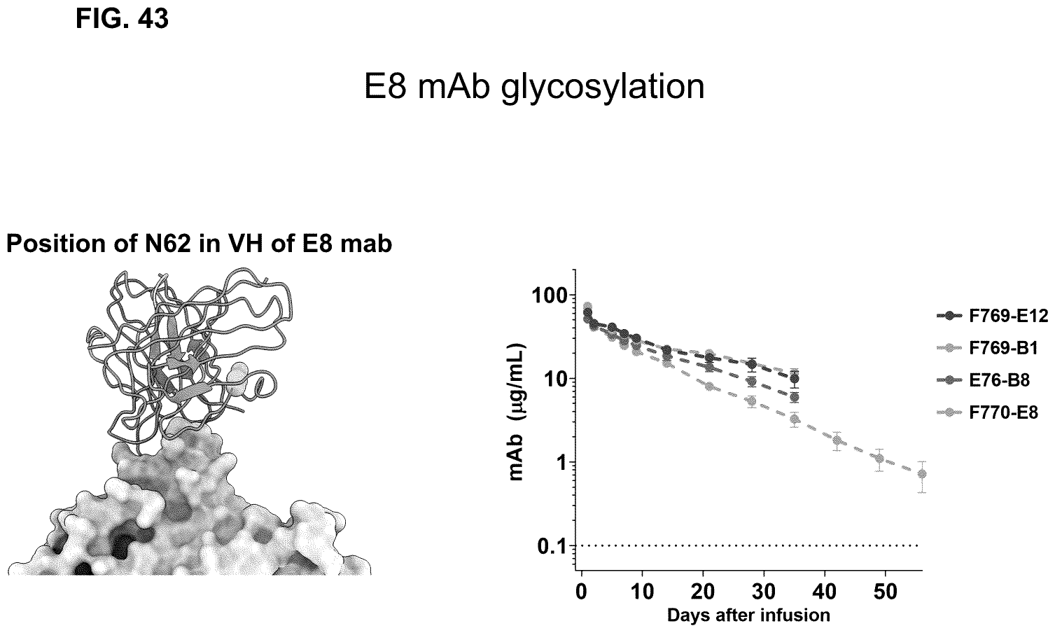

FIG. 43 shows the resulting serum concentration of E8 glycosylation mutant antibodies in g/mL.

All publications, patents and patent applications mentioned in this application are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-6. (canceled)

7. An antigen binding polypeptide complex comprising a first polypeptide and a second polypeptide;
wherein the first polypeptide has a structure represented by:
VL1-L1-VL2-L2-VH2-L3-VH1-L4-Fc;
VL1-L1-VH2-L2-VL2-L3-VH1-L4-Fc;
VH1-L5-VH2-L6-VL2-L7-VL1-L8-Fc; or
VH1-L5-VL2-L6-VH2-L7-VL1-L8-Fc;
wherein the second polypeptide has a structure represented by:
Fc;
VL3-L9-VL4-L10-VH4-L11-VH3-L12-Fc;
VL3-L9-VH4-L10-VL4-L11-VH3-L12-Fc;
VH3-L13-VH4-L14-VL4-L15-VL3-L16-Fc; or
VH3-L13-VL4-L14-VH4-L15-VL3-L16-Fc;
wherein:
VL1 is a first immunoglobulin light chain variable region that specifically binds to a SARS-CoV-2 protein;
VL2 is a second immunoglobulin light chain variable region that specifically binds to a SARS-CoV-2 protein;
VL3 is a third immunoglobulin light chain variable region that specifically binds to a SARS-CoV-2 protein;
VL4 is a fourth immunoglobulin light chain variable region that specifically binds to a SARS-CoV-2 protein;
VH1 is a first immunoglobulin heavy chain variable region that specifically binds to a SARS-CoV-2 protein;
VH2 is a second immunoglobulin heavy chain variable region that specifically binds to a SARS-CoV-2 protein;
VH3 is a third immunoglobulin heavy chain variable region that specifically binds to a SARS-CoV-2 protein;
VH4 is a fourth immunoglobulin heavy chain variable region that specifically binds to a SARS-CoV-2 protein;
Fc is a region comprising an immunoglobulin heavy chain constant region 2 (CH2), an immunoglobulin heavy chain constant region 3 (CH3), and optionally, an immunoglobulin hinge; and
L1-L16 are amino acid linkers.

8. An antigen binding polypeptide complex according to claim 7 comprising a first polypeptide and a second polypeptide;
wherein the first polypeptide has a structure represented by:
VL1-L1-VL2-L2-VH2-L3-VH1-L4-Fc; or
VH1-L5-VH2-L6-VL2-L7-VL1-L8-Fc;
wherein the second polypeptide has a structure represented by:
Fc;
VL3-L9-VL4-L10-VH4-L11-VH3-L12-Fc; or
VH3-L13-VH4-L14-VL4-L15-VL3-L16-Fc;
wherein:
VL1 is a first immunoglobulin light chain variable region that specifically binds to a SARS-CoV-2 protein;
VL2 is a second immunoglobulin light chain variable region that specifically binds to a SARS-CoV-2 protein;
VL3 is a third immunoglobulin light chain variable region that specifically binds to a SARS-CoV-2 protein;
VL4 is a fourth immunoglobulin light chain variable region that specifically binds to a SARS-CoV-2 protein;
VH1 is a first immunoglobulin heavy chain variable region that specifically binds to a SARS-CoV-2 protein;
VH2 is a second immunoglobulin heavy chain variable region that specifically binds to a SARS-CoV-2 protein;
VH3 is a third immunoglobulin heavy chain variable region that specifically binds to a SARS-CoV-2 protein;
VH4 is a fourth immunoglobulin heavy chain variable region that specifically binds to a SARS-CoV-2 protein;
Fc is a region comprising an immunoglobulin heavy chain constant region 2 (CH2), an immunoglobulin heavy chain constant region 3 (CH3), and optionally, an immunoglobulin hinge; and
L1-L16 are amino acid linkers.

9-14. (canceled)

15. An antigen binding polypeptide complex comprising a first polypeptide and a second polypeptide;
wherein the first polypeptide has a structure represented by:
VL1-L1-VL2-L2-VH2-L3-VH1-L4-CH1-L5-CL-L6-Fc;
VL1-L1-VH2-L2-VL2-L3-VH1-L4-CH1-L5-CL-L6-Fc;
VH1-L7-VH2-L8-VL2-L9-VL1-L10-CH1-L11-CL-L12-Fc;
VH1-L7-VL2-L8-VH2-L9-VL1-L10-CH1-L11-CL-L12-Fc;
VL1-L13-VL2-L14-VH2-L15-VH1-L16-CL-L17-CH1-L18-Fc;
VL1-L13-VH2-L14-VL2-L15-VH1-L16-CL-L17-CH1-L18-Fc;
VH1-L19-VH2-L20-VL2-L21-VL1-L22-CL-L23-CH1-L24-Fc; or
VH1-L19-VL2-L20-VH2-L21-VL1-L22-CL-L23-CH1-L24-Fc;
wherein the second polypeptide has a structure represented by:
Fc;
VL3-L25-VL4-L26-VH4-L27-VH3-L28-CH1-L29-CL-L30-Fc;
VL3-L25-VH4-L26-VL4-L27-VH3-L28-CH1-L29-CL-L30-Fc;
VH3-L31-VH4-L32-VL4-L33-VL3-L34-CH1-L35-CL-L36-Fc;
VH3-L31-VL4-L32-VH4-L33-VL3-L34-CH1-L35-CL-L36-Fc;
VL3-L37-VL4-L38-VH4-L39-VH3-L40-CL-L41-CH1-L42-Fc;
VL3-L37-VH4-L38-VL4-L39-VH3-L40-CL-L41-CH1-L42-Fc;
VH3-L43-VH4-L44-VL4-L45-VL3-L46-CL-L47-CH1-L48-Fc; or
VH3-L43-VL4-L44-VH4-L45-VL3-L46-CL-L47-CH1-L48-Fc;
wherein:
VL1 is a first immunoglobulin light chain variable region that specifically binds to a SARS-CoV-2 protein;
VL2 is a second immunoglobulin light chain variable region that specifically binds to a SARS-CoV-2 protein;
VL3 is a third immunoglobulin light chain variable region that specifically binds to a SARS-CoV-2 protein;
VL4 is a fourth immunoglobulin light chain variable region that specifically binds to a SARS-CoV-2 protein;
VH1 is a first immunoglobulin heavy chain variable region that specifically binds to a SARS-CoV-2 protein;
VH2 is a second immunoglobulin heavy chain variable region that specifically binds to a SARS-CoV-2 protein;
VH3 is a third immunoglobulin heavy chain variable region that specifically binds to a SARS-CoV-2 protein;
VH4 is a fourth immunoglobulin heavy chain variable region that specifically binds to a SARS-CoV-2 protein;
CH1 is an immunoglobulin heavy chain constant region 1;
CL is an immunoglobulin light chain constant region;
Fc is a region comprising an immunoglobulin heavy chain constant region 2 (CH2), an immunoglobulin heavy chain constant region 3 (CH3), and optionally, an immunoglobulin hinge; and
L1-L48 are amino acid linkers.

16. An antigen binding polypeptide complex according to claim 15 comprising a first polypeptide and a second polypeptide;
wherein the first polypeptide has a structure represented by:
VL1-L1-VL2-L2-VH2-L3-VH1-L4-CH1-L5-CL-L6-Fc;
VH1-L7-VH2-L8-VL2-L9-VL1-L10-CH1-L11-CL-L12-Fc;
VL1-L13-VL2-L14-VH2-L15-VH1-L16-CL-L17-CH1-L18-Fc; or
VH1-L19-VH2-L20-VL2-L21-VL1-L22-CL-L23-CH1-L24-Fc;
wherein the second polypeptide has a structure represented by:
Fc;
VL3-L25-VL4-L26-VH4-L27-VH3-L28-CH1-L29-CL-L30-Fc;
VH3-L31-VH4-L32-VL4-L33-VL3-L34-CH1-L35-CL-L36-Fc;
VL3-L37-VL4-L38-VH4-L39-VH3-L40-CL-L41-CH1-L42-Fc; or
VH3-L43-VH4-L44-VL4-L45-VL3-L46-CL-L47-CH1-L48-Fc;
wherein:
VL1 is a first immunoglobulin light chain variable region that specifically binds to a SARS-CoV-2 protein;
VL2 is a second immunoglobulin light chain variable region that specifically binds to a SARS-CoV-2 protein;
VL3 is a third immunoglobulin light chain variable region that specifically binds to a SARS-CoV-2 protein;
VL4 is a fourth immunoglobulin light chain variable region that specifically binds to a SARS-CoV-2 protein;
VH1 is a first immunoglobulin heavy chain variable region that specifically binds to a SARS-CoV-2 protein;
VH2 is a second immunoglobulin heavy chain variable region that specifically binds to a SARS-CoV-2 protein;
VH3 is a third immunoglobulin heavy chain variable region that specifically binds to a SARS-CoV-2 protein;
VH4 is a fourth immunoglobulin heavy chain variable region that specifically binds to a SARS-CoV-2 protein;
CH1 is an immunoglobulin heavy chain constant region 1;
CL is an immunoglobulin light chain constant region;
Fc is a region comprising an immunoglobulin heavy chain constant region 2 (CH2), an immunoglobulin heavy chain constant region 3 (CH3), and optionally, an immunoglobulin hinge; and
L1-L48 are amino acid linkers.

17. An antigen binding polypeptide or antigen binding polypeptide complex comprising a polypeptide having a structure represented by:
VL1-L1-VL2-L2-VH2-L3-VH1-L4-Fc-L5-Fc;
VL1-L1-VH2-L2-VL2-L3-VH1-L4-Fc-L5-Fc;
VH1-L6-VH2-L7-VL2-L8-VL1-L9-Fc-L10-Fc; or
VH1-L6-VL2-L7-VH2-L8-VL1-L9-Fc-L10-Fc;
wherein:
VL1 is a first immunoglobulin light chain variable region that specifically binds to a SARS-CoV-2 protein;
VL2 is a second immunoglobulin light chain variable region that specifically binds a SARS-CoV-2 protein;
VH1 is a first immunoglobulin heavy chain variable region that specifically binds to a SARS-CoV-2 protein;
VH2 is a second immunoglobulin heavy chain variable region that specifically binds to a SARS-CoV-2 protein;
Fc is a region comprising an immunoglobulin heavy chain constant region 2 (CH2), an immunoglobulin heavy chain constant region 3 (CH3), and optionally, an immunoglobulin hinge; and
L1-L10 are amino acid linkers.

18. An antigen binding polypeptide or antigen binding polypeptide complex according to claim 17 comprising a polypeptide having a structure represented by:
VL1-L1-VL2-L2-VH2-L3-VH1-L4-Fc-L5-Fc; or
VH1-L6-VH2-L7-VL2-L8-VL1-L9-Fc-L10-Fc;
wherein:
VL1 is a first immunoglobulin light chain variable region that specifically binds to a SARS-CoV-2 protein;
VL2 is a second immunoglobulin light chain variable region that specifically binds a SARS-CoV-2 protein;
VH1 is a first immunoglobulin heavy chain variable region that specifically binds to a SARS-CoV-2 protein;
VH2 is a second immunoglobulin heavy chain variable region that specifically binds to a SARS-CoV-2 protein;
Fc is a region comprising an immunoglobulin heavy chain constant region 2 (CH2), an immunoglobulin heavy chain constant region 3 (CH3), and optionally, an immunoglobulin hinge; and
L1-L10 are amino acid linkers.

19-27. (canceled)

28. The antigen binding polypeptide complex of claim 7, wherein linkers L1-L16 each independently have a length of from 0 amino acids to about 50 amino acids.

29. The antigen binding polypeptide complex of claim 7, wherein linkers L1-L16 not having a length of 0 amino acids each independently comprise an amino acid sequence having at least 80%, at least 85%, at least 90%, or at least 95% identity to any one of SEQ ID NOs:1-34, wherein SEQ ID NO:1 has the amino acid sequence of G, SEQ ID NO:2 has the amino acid sequence of A, SEQ ID NO:3 has the amino acid sequence of GSS and SEQ ID NO:4 has the amino acid sequence of ASG.

30-31. (canceled)

32. The antigen binding polypeptide complex of claim 7, wherein the Fc region comprises at least one knob-into-hole modification or Fc effector function knockout mutation.

33. The antigen binding polypeptide complex of claim 32, wherein the antigen binding polypeptide complex is an IgG1 or IgG4 antibody and the knob-into-hole modification comprises:
(i) knob substitutions of S354C and T366W and hole substitutions of Y349C, T366S, L368A and Y407V;
(ii) hole substitutions of M428L and N434S or N434A;
(iii) hole substitutions of M252Y, S254T and T256E; or
(iv) a combination thereof;
based on the EU numbering scheme.

34. The antigen binding polypeptide complex of claim 32, wherein the antigen binding polypeptide complex is an IgG1 or IgG4 antibody and the Fc effector function knockout mutation is L234A, L235A, P239A or a combination thereof, based on the EU numbering scheme.

35-38. (canceled)

39. The antigen binding polypeptide complex of claim 7 that specifically binds to a SARS-CoV-2 protein with an equilibrium dissociation constant (K_{D}) of from about 10 μM to about 1 μM.

40. The antigen binding polypeptide complex of claim 7, wherein the antigen binding polypeptide complex is bispecific, trispecific or tetraspecific and has greater neutralization potency against SARS-CoV-2 virus than a monospecific antigen binding polypeptide complex that specifically binds to one of the same antigens as the bispecific, trispecific or tetraspecific antigen binding polypeptide complex.

41. The antigen binding polypeptide complex of claim 7, wherein the antigen binding polypeptide complex is bispecific, trispecific or tetraspecific and has greater neutralization potency against SARS-CoV-2 virus than a mixture of monospecific antigen binding polypeptide complexes that specifically bind to the same antigens as the bispecific, trispecific or tetraspecific antigen binding polypeptide complex.

42. The antigen binding polypeptide complex of claim 41, wherein the SARS-CoV-2 virus is the original Wuhan strain (WA1), D614G spike protein mutant (D614G), Alpha variant (B.1.1.7), Beta variant (B.1.351), Gamma variant (P.1), Delta variant, Epsilon variant (B.1.427), or Omicron variant (B.1.1.529), or a combination thereof.

43. An antibody or antigen binding fragment thereof comprising the antigen binding polypeptide complex of claim 7.

44. The antibody or antigen binding fragment thereof of claim 43, wherein the antibody is IgG, IgM, IgE, IgA or IgD.

45. The antibody or antigen binding fragment thereof of claim 44, wherein the IgG is IgG1, IgG2, IgG3 or IgG4.

46. The antibody or antigen binding fragment thereof of claim 43, wherein the antigen binding fragment is a Fab, scFab, Fab′, F(ab′)₂, Fv or scFv.

47. The antibody or antigen binding fragment thereof of claim 43, wherein the antibody is human or humanized.

48. The antibody or antigen binding fragment thereof of claim 43, wherein the antibody is a monoclonal antibody, grafted antibody, or chimeric antibody.

49. An anti-SARS-CoV-2 multivalent antibody that exhibits improved prevention of immune escape compared to (i) a monovalent anti-SARS-CoV-2 antibody that binds to one of the same antigens as the anti-SARS-CoV-2 multivalent antibody, and/or (ii) a combination of monovalent anti-SARS-CoV-2 antibodies that binds to the same antigens as the anti-SARS-CoV-2 multivalent antibody.

50-51. (canceled)

52. A pharmaceutical composition comprising the antigen binding polypeptide complex of claim 7 and a pharmaceutically acceptable carrier.

53-65. (canceled)

66. A kit comprising the antigen binding polypeptide complex of claim 7.

67-72. (canceled)

73. A method of preventing or treating a SARS-CoV-2 viral infection in a subject in need thereof, comprising administering to the subject a therapeutically effective amount of the antigen binding polypeptide complex of claim 7.

74. A method of preventing or treating coronavirus disease 2019 (COVID-19) in a subject in need thereof, comprising administering to the subject a therapeutically effective amount of the antigen binding polypeptide complex of claim 7.

75. A method of diagnosing a subject as being infected with a SARS-CoV-2 virus or suspected of being infected with a SARS-CoV-2 virus, comprising (i) contacting a sample obtained from the subject with the antigen binding polypeptide complex of claim 7; (ii) detecting the presence or absence of a virus complex which contains the polypeptide complex and a SARS-CoV-2 virus, virion or fragment thereof; and (iii) diagnosing the subject as being infected with a SARS-CoV-2 virus or suspected of being infected with a SARS-CoV-2 virus when the presence of the virus complex is detected.

76. A method of diagnosing a subject as not being infected with a SARS-CoV-2 virus or not suspected of being infected with a SARS-CoV-2 virus, comprising (i) contacting a sample obtained from the subject with the antigen binding polypeptide complex of claim 7; (ii) detecting the presence or absence of a virus complex which contains the polypeptide complex and a SARS-CoV-2 virus, virion or fragment thereof; and (iii) diagnosing the subject as not being infected with a SARS-CoV-2 virus or not suspected of being infected with a SARS-CoV-2 virus when the presence of the virus complex is not detected.

77-83. (canceled)

84. A method of preventing immune escape of a SARS-CoV-2 virus in a subject infected with the SARS-CoV-2 virus, comprising administering to the subject an effective amount of an anti-SARS-CoV-2 multivalent antibody that exhibits improved prevention of immune escape compared to (i) a monovalent anti-SARS-CoV-2 antibody that binds to one of the same antigens as the anti-SARS-CoV-2 multivalent antibody, and/or (ii) a combination of monovalent anti-SARS-CoV-2 antibodies that binds to the same antigens as the anti-SARS-CoV-2 multivalent antibody.

85. A method of increasing neutralization potency against a SARS-CoV-2 virus in a subject infected with the SARS-CoV-2 virus, comprising administering to the subject an effective amount of an anti-SARS-CoV-2 multivalent antibody that exhibits increased neutralization potency compared to (i) a monovalent anti-SARS-CoV-2 antibody that binds to one of the same antigens as the anti-SARS-CoV-2 multivalent antibody and/or (ii) a combination of monovalent anti-SARS-CoV-2 antibodies that binds to the same antigens as the anti-SARS-CoV-2 multivalent antibody.

86-128. (canceled)

129. A multispecific antibody selected from:
(a) an antigen binding polypeptide having a structure represented by:
VL1-L1-VH1 or VH1-L2-VL1;
wherein:
VL1 is a first immunoglobulin light chain variable region that specifically binds to a severe acute respiratory syndrome coronavirus 2 (SARS-CoV-2) protein;
VH1 is a first immunoglobulin heavy chain variable region that specifically binds to a SARS-CoV-2 protein; and
L1 and L2 are amino acid linkers; or
an antigen binding polypeptide having a structure represented by:
VL1-L1-VL2-L2-VH2-L3-VH1;
VL1-L1-VH2-L2-VL2-L3-VH1;
VH1-L4-VH2-L5-VL2-L6-VL1; or
VH1-L4-VL2-L5-VH2-L6-VL1;
wherein:
VL1 is a first immunoglobulin light chain variable region that specifically binds to a SARS-CoV-2 protein;
VL2 is a second immunoglobulin light chain variable region that specifically binds to a SARS-CoV-2 protein;
VH1 is a first immunoglobulin heavy chain variable region that specifically binds to a SARS-CoV-2 protein;
VH2 is a second immunoglobulin heavy chain variable region that specifically binds to a SARS-CoV-2 protein; and
L1-L6 are amino acid linkers; and
wherein (a) is an antigen binding polypeptide in an antigen binding polypeptide complex having no more than three polypeptide chains; or
(b) an antigen binding polypeptide having a structure represented by:
VL1-L1-VH1 or VH1-L2-VL1;
wherein:
VL1 is a first immunoglobulin light chain variable region that specifically binds to a SARS-CoV-2 protein;
VH1 is a first immunoglobulin heavy chain variable region that specifically binds to a SARS-CoV-2 protein; and
L1 and L2 are amino acid linkers; or
an antigen binding polypeptide having a structure represented by:
VL1-L1-VL2-L2-VH2-L3-VH1;
VL1-L1-VH2-L2-VL2-L3-VH1;
VH1-L4-VH2-L5-VL2-L6-VL1; or
VH1-L4-VL2-L5-VH2-L6-VL1;
wherein:
VL1 is a first immunoglobulin light chain variable region that specifically binds to a SARS-CoV-2 protein;
VL2 is a second immunoglobulin light chain variable region that specifically binds to a SARS-CoV-2 protein;
VH1 is a first immunoglobulin heavy chain variable region that specifically binds to a SARS-CoV-2 protein;
VH2 is a second immunoglobulin heavy chain variable region that specifically binds to a SARS-CoV-2 protein; and
L1-L6 are amino acid linkers;
wherein (b) is an antigen binding polypeptide in an antigen binding polypeptide complex having no more than three polypeptide chains; and
wherein the antigen binding polypeptide further comprises one or more immunoglobulin heavy chain constant region 1 (CH1) and/or one or more immunoglobulin light chain constant region (CL) between any one of the variable regions and/or amino acid linkers.

130-132. (canceled)
